(19) United States
(12) Patent Application Publication
(10) Pub. No.: US 2011/0184188 A1
(43) Pub. Date: Jul. 28, 2011

(54) Insecticidal Benzenedicarboxamide Derivative

(75) Inventors: Katsuaki Wada, Tochigi (JP); Yasushi Yoneta, Saitama (JP); Takuya Gomibuchi, Ibaraki (JP); Tetsuya Murata, Tochigi (JP); Sachio Kudo, Ibaraki (JP); Hidetoshi Kishikawa, Tochigi (JP); Katsuhiko Shibuya, Tochigi (JP); Eiichi Shimojo, Tochigi (JP); Akira Emoto, Tochigi (JP); Yoshitaka Sato, Ibaraki (JP); Rüdiger Fisher, Pulheim (DE); Christian Funke, Leichlinger (DE); Christian Arnold, Langenfeld (DE); Eva-Maria Franken, Leichlingen (DE); Olga Malsam, Rosrath (DE); Erich Sanwald, Kiel (DE); Ulrich Görgens, Ratingen (DE); Masashi Ataka, Saitama (JP); Udo Reckmann, Koln (DE); Christian Paulitz, am Taunus (DE); Tobias Kapferer, Dusseldoft (DE)

(73) Assignee: Bayer CropScience AG, Monheim (DE)

(21) Appl. No.: 13/056,518

(22) PCT Filed: Jul. 28, 2009
(86) PCT No.: PCT/EP2009/005439

§ 371 Date: Apr. 18, 2011

(30) Foreign Application Priority Data
Jul. 31, 2008 (JP) 2008197377

(51) Int. Cl. C07D 249/08; C07D 231/12

(52) U.S. Cl. 548/262.2; 548/373.1

(57) ABSTRACT

The present invention relates to a novel benzenedicarboxamide derivative and the use thereof as an insecticide having the formula (I) wherein the chemical groups W¹ to W⁹, and R¹ to R³ are as defined here-in.

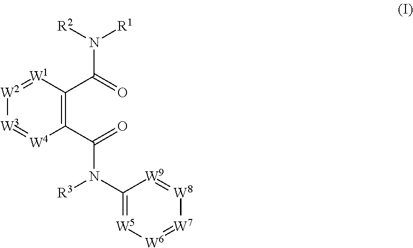

The present invention relates to a novel benzenedicarboxamide derivative and the use thereof as an insecticide.

It is known that certain benzenedicarboxamide derivatives provide a biological activity. For example, Document 1: JP11-240857 and its English equivalent EP-A-0936212; Document 2: JP 2001-64258 and its English equivalent EP-A-1188745; Document 3: JP 2001-64268 and its English equivalent EP-A-1195375; Document 4: JP 2001-131141 and its English equivalent EP-A-1006107; Document 5: JP2003-40864; Document 6: WO 01/21576 (WO01/21576); Document 7: WO03/110258; and Document 9: JP 2006-76990 and its English equivalent EP-A-17278049 describe that certain 1,2-benzenedicarboxamide derivatives can be used an insecticide; whereas Document 8: JP 59-163353 and its English equivalent EP-A-0119428 states that some of the 1,2-benzenedicarboxamide derivatives exhibit an action as a medical drug.

Since ecological and economic demands on modern plant treatment agents are continually increasing, particularly in respect to the amount applied, residue formation, selectivity, toxicity and favourable production methodology, and also because, for example, resistance problems can occur, there is the on-going task to develop new plant treatment agents that at least in certain areas are able to demonstrate advantages over known agents.

The inventors of the present invention devotedly conducted research to create a novel compound exhibiting higher effects and having a wide spectrum as an insecticide and lower toxicity. As a result they have found a novel benzenedicarboxamide, which exhibit higher activity as an insecticide and which has excellent insecticidal effect and is represented by the following formula (I):

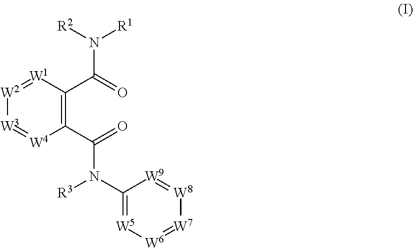

wherein
R¹ represents a hydrogen atom, or C₁₋₁₂ alkyl, C₂₋₈ alkenyl, C₂₋₈ alkynyl, C₃₋₈ cycloalkyl, C₁₋₁₂alkoxy, C₂₋₁₂ (total number of carbon atoms) dialkylamino, carboxy-C₁₋₁₂ alkyl, formyl-C₁₋₁₂ alkyl, hydroxyimino-C₁₋₁₂ alkyl, hydroxy-C₁₋₁₂ alkyl, C₂₋₁₂ (total number of carbon atoms) alkoxyalkyl, aminosulfonyl-C₁₋₆ alkyl, C₂₋₁₂ (total number of carbon atoms) alkylaminosulfonylalkyl, C₂₋₁₂ (total number of carbon atoms) alkylthioalkyl, C₂₋₁₂ (total number of carbon atoms) alkylsulfinylalkyl, C₂₋₁₂ (total number of carbon atoms) alkylsulfonylalkyl, C₄₋₁₂ (total number of carbon atoms) cycloalkylthioalkyl, C₄₋₁₂ (total number of carbon atoms) cycloalkylsulfinylalkyl, C₄₋₁₂ (total number of carbon atoms) cycloalkylsulfonylalkyl, C₄₋₁₀ (total number of carbon atoms) trialkylsilylalkyl, C₂₋₁₀ (total number of carbon atoms) alkoxycarbonylmethylaminocarbonylalkyl, C₂₋₁₀ (total number of carbon atoms) alkylcarbamoyloxyalkyl, C₂₋₁₀ (total number of carbon atoms) monoalkylcarbamoylalkyl, C₃₋₁₀ (total number of carbon atoms) monoalkenylcarbamoylalkyl, C₄₋₁₀ (total number of carbon atoms) cycloalkylcarbamoylalkyl, C₂₋₁₀ (total number of carbon atoms) alkoxycarbonylaminoalkyl, C₂₋₁₀ (total number of carbon atoms) dialkylaminosulfonylalkyl, benzyloxycarbonyl-C₁₋₆ alkyl, benzyloxy-C₁₋₆ alkyl, C₁₋₆ alkylthioaryl, C₁₋₆ alkylsulfinylaryl, C₁₋₆ alkylsulfonylaryl, C₁₋₆ alkylthioheteroaryl, C₁₋₆ alkylsulfinylheteroaryl, C₁₋₆ alkylsulfonylheteroaryl, C₄₋₁₄ (total number of carbon atoms) cycloalkylalkyl which may be substituted, or 5- or 6-membered heterocyclyl or 5- or 6-membered heterocyclyl-C₁₋₈ alkyl which may be substituted,
R² and R³ each independently represent a hydrogen atom, or C₁₋₁₂-alkyl, C₂₋₆-alkenyl, C₂₋₅-alkynyl, C₁₋₁₂-alkoxyalkyl or C₁₋₁₂-alkylthioalkyl which may be substituted,
R¹ and R² together with a nitrogen atom to which they are attached may form a 5- or 6-membered heterocyclic group,
W¹ represents a nitrogen atom or C—X¹,
W² represents a nitrogen atom or C—X²,
W³ represents a nitrogen atom or C—X³,
W⁴ represents a nitrogen atom or C—X⁴,
X¹, X², X³ and X⁴, which may be identical or different, represent a hydrogen atom, nitro, formyl, amino, cyano, halogen, carbamoyl, or C₁₋₁₂-alkyl, C₁₋₁₂-haloalkyl, C₁₋₁₂-alkylcarbonyl, C₁₋₁₂-alkoxycarbonyl, C₁₋₁₂-acylamino, C₁₋₁₂-alkoxy, C₁₋₁₂-haloalkoxy, C₁₋₁₂-alkylthio, C₁₋₁₂-alkylsulfinyl, C₁₋₁₂-alkylsulfonyl, C₁₋₁₂-haloalkylthio, C₁₋₁₂-haloalkylsulfinyl, C₁₋₁₂-haloalkylsulfonyl, C₃₋₈ cycloalkylthio, C₃₋₈ cycloalkylsulfinyl, C₃₋₈ cycloalkylsulfonyl, C₄₋₁₄ (total number of carbon atoms) cycloalkylalkylthio, C₄₋₁₄-(total number of carbon atoms) cycloalkylalkylsulfinyl, C₄₋₁₄ (total number of carbon atoms) cycloalkylalkylalkylsulfonyl, C₁₋₁₂-alkylsulfonyloxy, C₁₋₁₂-haloalkylsulfonyloxy, C₁₋₁₂-alkylaminosulfonyl, C₂₋₁₂ (total number of carbon atoms) dialkylaminosulfonyl, C₁₋₁₂-alkylamino, C₂₋₁₂ (total number of carbon atoms) dialkylamino, C₁₋₁₂ alkylcarbamoyl, C₂₋₁₂ (total number of carbon atoms) dialkylcarbamoyl, C₂₋₆-alkenyl, C₂₋₆-alkenyloxy, C₂₋₆-alkynyl, C₂₋₆-alkynyloxy, C₃₋₈-cycloalkyl, C₄₋₁₄-cycloalkyloxy which may be substituted, or aryl, aryloxy, arylthio, aryl-C₁₋₆-alkyl, aryl-C₁₋₆-alkoxy which may be substituted, aryl C₁₋₆-alkylthio, heteroaryl, heterocyclyl, heterocyclyloxy, heterocyclylthio or heterocyclyl-C₁₋₆-alkoxy which may be substituted by nitro, formyl, cyano, halogen, and C₁₋₆-alkyl, C₁₋₆-haloalkyl, C₁₋₆-alkylcarbonyl, C₁₋₆-alkoxycarbonyl, C₁₋₆-alkoxy, C₁₋₆-haloalkoxy, C₁₋₆-alkylthio or C₁₋₆ haloalkylthio which may be substituted, or wherein X¹ together with X², or X² together with X³, or X³ together with X⁴ may form a 5- or 6-membered carbon ring, or may form a heterocyclic group which contains or is constituted by a combination of oxygen, sulfur, nitrogen which may be substituted by C₁₋₂ alkyl, and carbon which may be substituted by halogen,
W⁵ represents a nitrogen atom, C—Y¹ or a group C-(A)r-Q,
W⁶ represents a nitrogen atom, C—Y² or a group C-(A)r-Q,
W⁷ represents a nitrogen atom, C—Y³ or a group C-(A)r-Q,
W⁸ represents a nitrogen atom, C—Y⁴ or a group C-(A)r-Q,
W⁹ represents a nitrogen atom, C—Y⁵ or a group C-(A)r-Q, under the proviso that at least one of W⁵ to W⁹ necessarily represents C-(A)r-Q,
Y¹, Y², Y³, Y⁴ and Y⁵, which may be identical or different, represent a hydrogen atom, nitro, formyl, carbamoyl, amino, cyano, halogen, or C₁₋₁₂-alkyl, C₁₋₁₂-haloalkyl, C₁₋₁₂-alkylcarbonyl, C₁₋₁₂-alkoxycarbonyl, C₁₋₁₂-acylamino, C₁₋₁₂-alkoxy, C₁₋₁₂-haloalkoxy, C₂₋₁₂ (total number of carbon atoms) alkoxycarbonylalkyl, C₁₋₁₂-alkylthio, C₁₋₁₂-alkylsulfinyl, C₁₋₁₂-alkylsulfonyl, C₁₋₁₂-haloalkylthio, C₂₋₁₂ (total number of carbon atoms) alkylthioalkyl, C₁₋₁₂-monoalkylamino, C₂₋₁₂-(total number of carbon atoms) dialkylamino, C₁₋₁₂-alkylcarbamoyl, C₂₋₁₂ (total number of carbon atoms) dialkylcarbamoyl, C₂₋₆-alkenyl, C₂₋₆-alkenyloxy, C₂₋₆-alkynyl, C₂₋₆-alkynyloxy, C₃₋₈ cycloalkyl, C₄₋₁₄-cycloalkyloxy, C₄₋₁₄-(total number of carbon atoms) cycloalkylalkyl, C₄₋₁₄ (total number of carbon atoms) cycloalkylalkoxy which may be substituted, or aryl, aryloxy, arylthio, aryl C₁₋₆ alkyl, aryl C₁₋₆ alkoxy, aryl C₁₋₆ alkylthio, heteroaryl, heterocyclyl, heterocyclyloxy, heterocyclylthio or heterocyclyl C₁₋₆ alkoxy which may be substituted by at least one selected from the group consisting of formyl, cyano, halogen, C₁₋₆ alkyl, C₁₋₆ haloalkyl, C₁₋₆ alkylcarbonyl, C₁₋₆ alkoxycarbonyl, C₁₋₆ alkoxy, C₁₋₆ haloalkoxy, C₁₋₆ alkylthio and C₁₋₆ haloalkylthio,
A represents O, S, SO, SO₂, NH, N(CH₃), CH(CN), C(═N—OCH₃), C(═O), CH(OH), CH(CH₃), CH(CF₃), C(CF₃)₂, CH(CO₂CH₃), CH(CO₂C₂H₅), or alkylene which may be interrupted by O, S, SO, SO₂, NH, N(CH₃), CH(CN), C(═N—OCH₃), C(═O), CH(OH), CH(CH₃), CH(CF₃), C(CF₃)₂, CH(CO₂CH₃) or CH(CO₂C₂H₅), or
A represents CH(Hal), CHal₂, C(CH₃)₂
r represents 0, 1, 2, 3, 4 or 5, preferably 0, 1, 2 or 3, more preferably 1, 2, and 3;
Q represents a 5- or 6-membered heterocyclic group which may be substituted, preferably selected among the following groups Q1 to Q67:

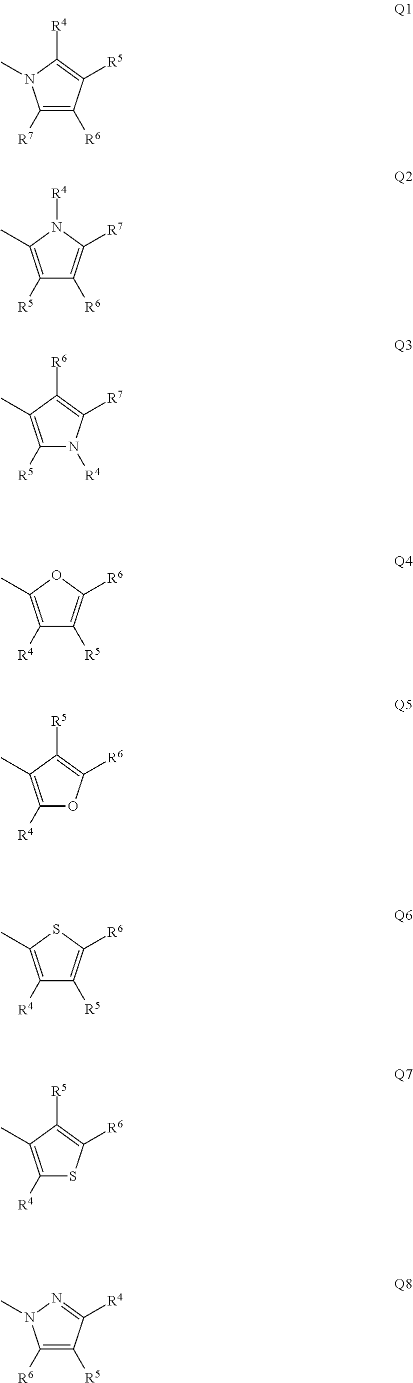

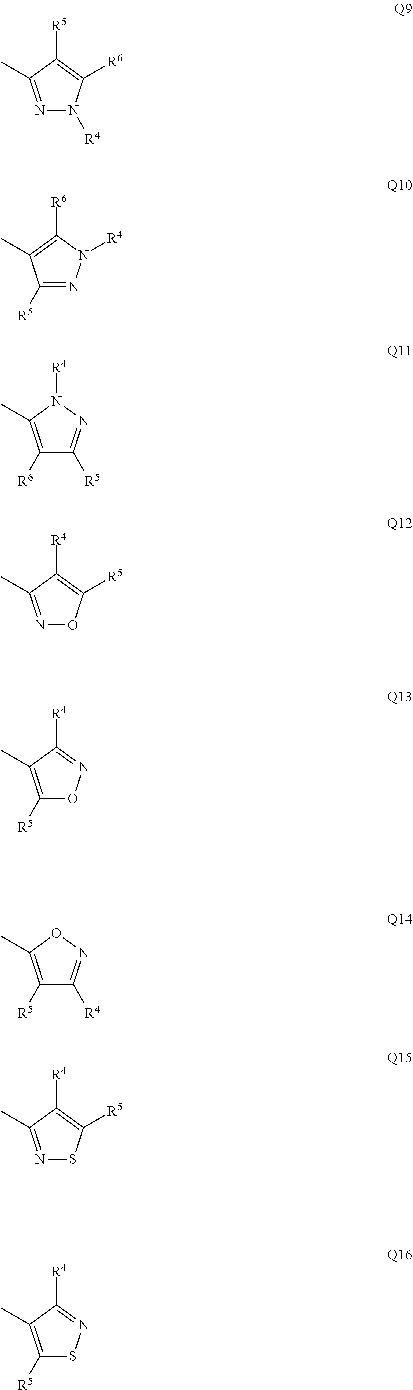

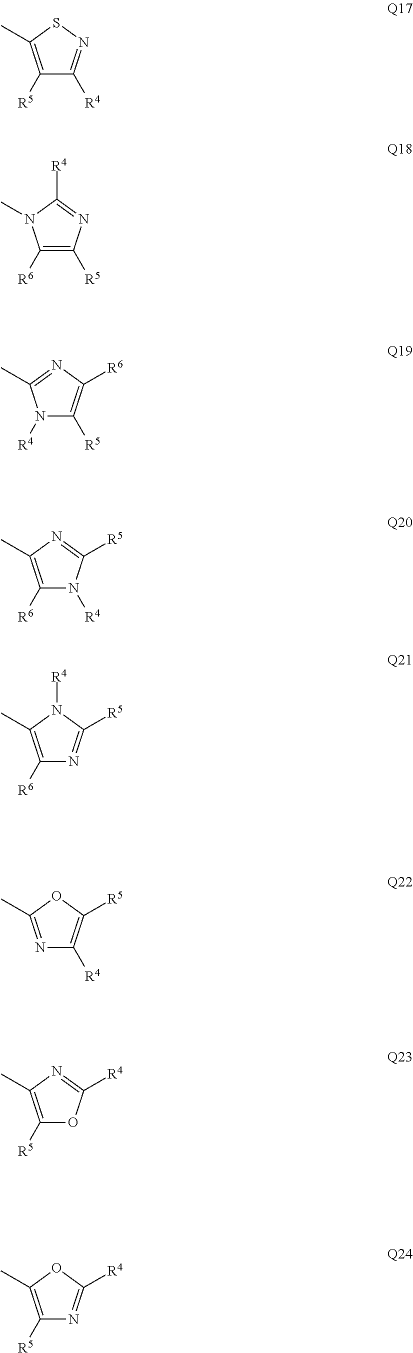

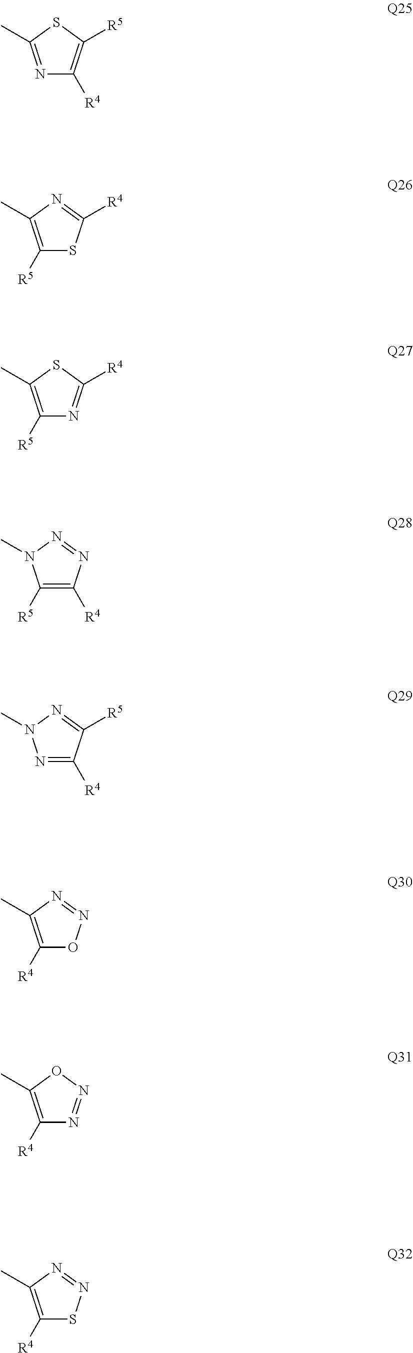

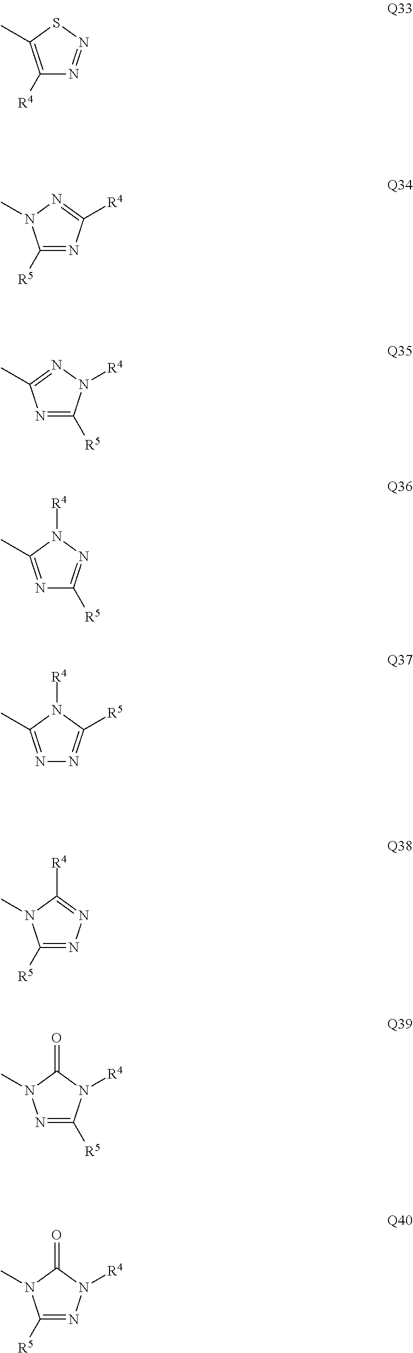

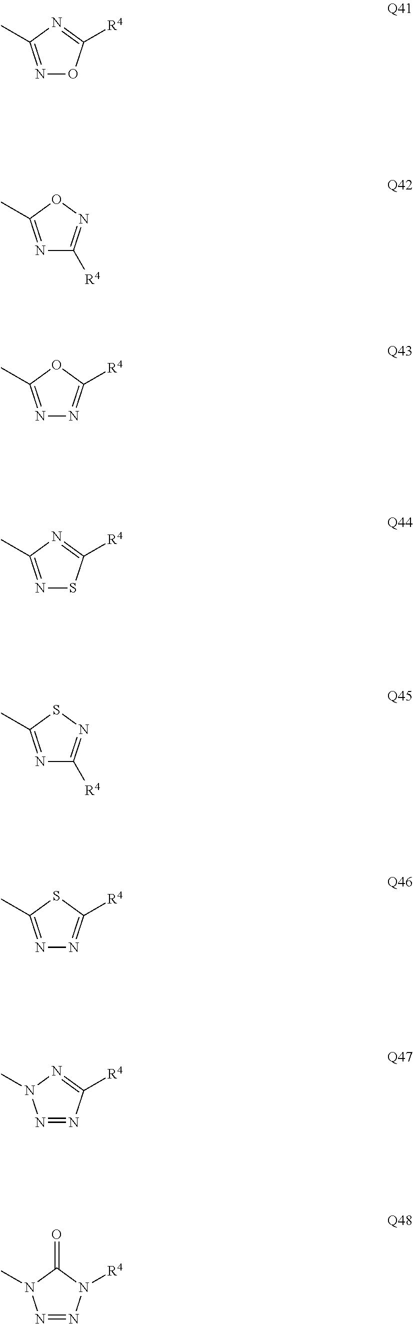

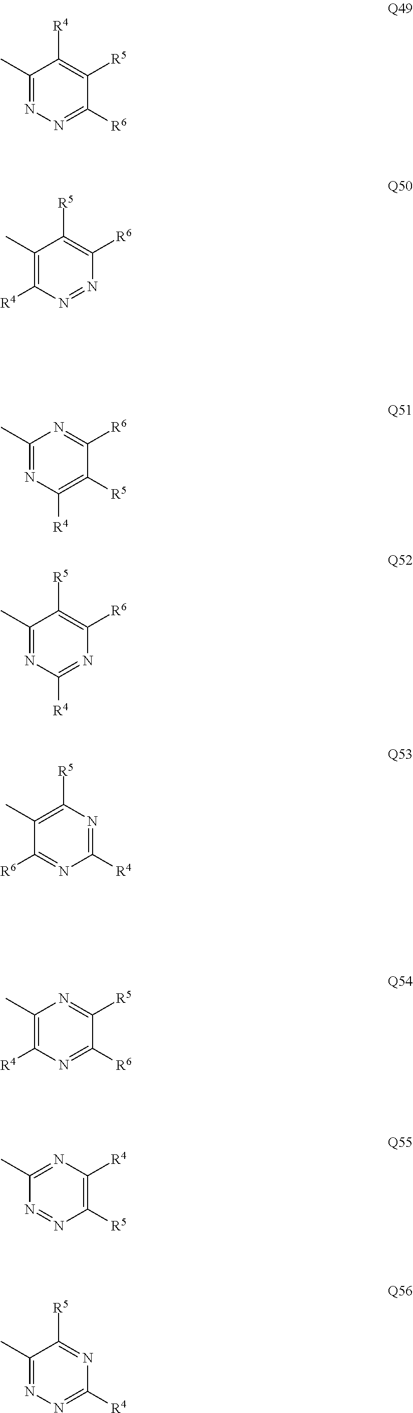

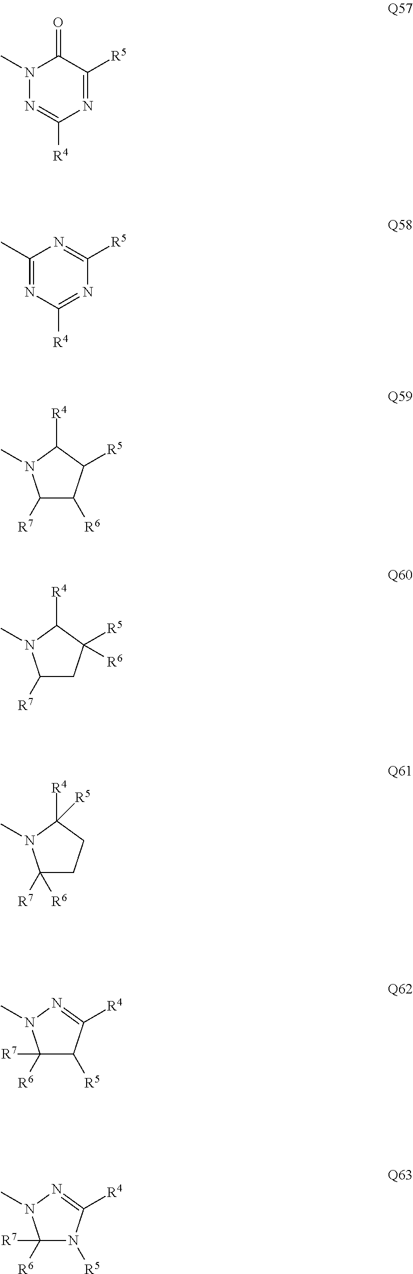

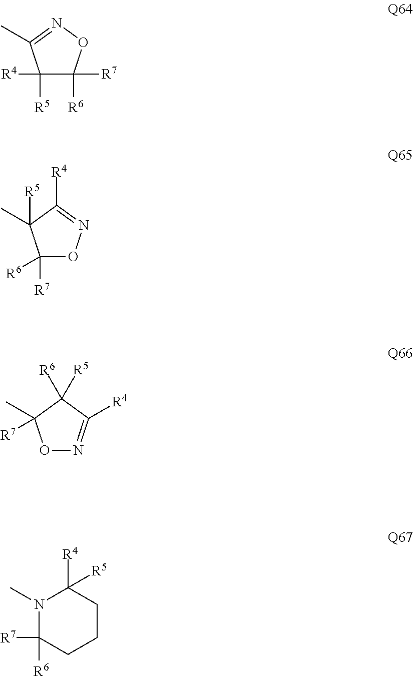

wherein
R⁴, R⁵, R⁶ and R⁷, which may be identical or different, represent a hydrogen atom, halogen, amino, cyano, nitro, or C₁₋₆ alkyl, C₁₋₁₀ haloalkyl, C₂₋₆ alkynyl, C₃₋₈ cyaloalkyl, C₁₋₆ alkoxy, C₁₋₆ haloalkoxy, C₁₋₆ alkylthio, C₁₋₆ haloalkylthio, C₁₋₆ alkylsulfinyl, C₁₋₆ haloalkylsulfinyl, C₁₋₆ alkylsulfonyl, C₁₋₆ haloalkylsulfonyl, C₁₋₆ alkoxycarbonyl, C₁₋₆ alkylcarbonyl, C₁₋₆ haloalkylcarbonyl, C₁₋₆ acylamino, C₁₋₆ haloacylamino, C₁₋₆ monoalkylcarbamoyl, C₂₋₈ (total number of carbon atoms) dialkylcarbamoyl, hydroxyimino-C₁₋₆ alkyl, C₂₋₈ (total number of carbon atoms) alkoxyiminoalkyl, hydroxyimino-C₁₋₆ haloalkyl, C₂₋₈ (total number of carbon atoms) alkoxyiminohaloalkyl which may be substituted, or phenylcarbamoyl which may be substituted by at least one selected from the group consisting of halogen, cyano, C₁₋₄ alkyl, C₁₋₆ haloalkyl, C₁₋₆ alkoxy, C₁₋₄ haloalkoxy, C₁₋₄ alkylthio and C₁₋₄ haloalkylthio, or a phenyl or heterocyclic group which may be substituted by at least one selected from the group consisting of halogen, cyano, and C₁₋₆ alkyl, C₂₋₆ alkenyl, C₁₋₆ haloalkyl, C₁₋₆ alkoxy, C₁₋₆ haloalkoxy, C₁₋₆ alkylthio, C₁₋₆ haloalkylthio, C₂₋₅ (total number of carbon atoms) alkylthioalkyl, C₁₋₆ alkylcarbonyl and C₁₋₆ alkoxycarbonyl which may be substituted, preferably R⁴, R⁵, R⁶ and R⁷, which may be identical or different, represent a hydrogen atom, halogen, amino, cyano, nitro, or C₁₋₄ alkyl, C₁₋₈ haloalkyl, C₂₋₄ alkynyl, C₃₋₆ cycloalkyl, C₁₋₄ alkoxy, C₁₋₄ haloalkoxy, C₁₋₄ alkylthio, C₁₋₄ haloalkylthio, C₁₋₄ alkylsulfinyl, C₁₋₄ haloalkylsulfinyl, C₁₋₄ alkylsulfonyl, C₁₋₄ haloalkylsulfonyl, C₁₋₄ alkoxycarbonyl, C₁₋₄ alkylcarbonyl, C₁₋₄ haloalkylcarbonyl, C₁₋₄ acylamino, C₁₋₄ haloacylamino, C₁₋₄ monoalkylcarbamoyl, C₂₋₆ (total number of carbon atoms) dialkylcarbamoyl, hydroxyimino-C₁₋₄ alkyl, C₂₋₆ (total number of carbon atoms) alkoxyiminoalkyl, hydroxyimino-C₁₋₄ haloalkyl, C₂₋₆ (total number of carbon atoms) alkoxyiminohaloalkyl which may be substituted, or phenylcarbamoyl which may be substituted by at least one selected from the group consisting of halogen, cyano, C₁₋₂ alkyl, C₁₋₄ haloalkyl, C₁₋₂ alkoxy, C₁₋₂ haloalkoxy, C₁₋₂ alkylthio and C₁₋₂ haloalkylthio, or a phenyl or heterocyclic group which may be substituted by at least one selected from the group consisting of halogen, cyano, and C₁₋₄ alkyl, C₂₋₄ alkenyl, C₁₋₄ haloalkyl, C₁₋₂ alkoxy, C₁₋₄. haloalkoxy, C₁₋₄ alkylthio, C₁₋₄ haloalkylthio, C₂₋₆ (total number of carbon atoms) alkylthioalkyl, C₁₋₄ alkylcarbonyl and C₁₋₄ alkoxycarbonyl which may be substituted.

Compounds of formula (I) as defined before are preferred, wherein
R¹ represents a hydrogen atom, or C₁₋₈ alkyl, C₂₋₈ alkenyl, C₂₋₈ alkynyl, C₃₋₈ cycloalkyl, C₁₋₈ alkoxy, C₂₋₈ (total number of carbon atoms) dialkylamino, carboxy-C₁₋₄ alkyl, formyl-C₁₋₆ alkyl, hydroxyimino-C₁₋₆ alkyl, hydroxy-C₁₋₄ alkyl, C₂₋₈ (total number of carbon atoms) alkoxyalkyl, aminosulfonyl-C₁₋₆ alkyl, C₂₋₁₀ (total number of carbon atoms) alkylaminosulfonylalkyl, C₂₋₁₀ (total number of carbon atoms) alkylthioalkyl, C₂₋₁₀ (total number of carbon atoms) alkylsulfinylalkyl, C₂₋₁₀ (total number of carbon atoms) alkylsulfonylalkyl, C₄₋₁₂ (total number of carbon atoms) cycloalkylthioalkyl, C₄₋₁₂ (total number of carbon atoms) cycloalkylsulfinylalkyl, C₄₋₁₂ (total number of carbon atoms) cycloalkylsulfonylalkyl, C₄₋₁₀ (total number of carbon atoms) trialkylsilylalkyl, C₂₋₁₀ (total number of carbon atoms) alkoxycarbonylmethylaminocarbonylalkyl, C₂₋₁₀ (total number of carbon atoms) alkylcarbamoyloxyalkyl, C₂₋₁₀ (total number of carbon atoms) monoalkylcarbamoylalkyl, C₃₋₁₀ (total number of carbon atoms) monoalkenylcarbamoylalkyl, C₄₋₁₀ (total number of carbon atoms) cycloalkylcarbamoylalkyl, C₂₋₁₀ (total number of carbon atoms) alkoxycarbonylaminoalkyl, C₂₋₁₀ (total number of carbon atoms) dialkylaminosulfonylalkyl, benzyloxycarbonyl-C₁₋₆ alkyl, benzyloxy-C₁₋₆ alkyl, C₁₋₄ alkylthioaryl, C₁₋₄ alkylsulfinylaryl, C₁₋₄ alkylsulfonylaryl, C₁₋₄ alkylthioheteroaryl, C₁₋₄ alkylsulfinylheteroaryl, C₁₋₄ alkylsulfonylheteroaryl, C₃₋₁₀ (total number of carbon atoms) cycloalkylalkyl which may be substituted, or 5- or 6-membered heterocyclyl or 5- or 6-membered heterocyclyl-C₁₋₆ alkyl which may be substituted, preferably R¹ represents a hydrogen atom, or C₁₋₆ alkyl, C₂₋₆ alkenyl, C₂₋₆ alkynyl, C₃₋₆ cycloalkyl, C₁₋₆ alkoxy, C₂₋₆ (total number of carbon atoms) dialkylamino, carboxy-C₁₋₄ alkyl, formyl-C₁₋₄ alkyl, hydroxyimino-C₁₋₄ alkyl, hydroxy-C₁₋₄ alkyl, C₂₋₆ (total number of carbon atoms) alkoxyalkyl, aminosulfonyl-C₁₋₄ alkyl, C₂₋₈ (total number of carbon atoms) alkylaminosulfonylalkyl, C₂₋₈ (total number of carbon atoms) alkylthioalkyl, C₂₋₈ (total number of carbon atoms) alkylsulfinylalkyl, C₂₋₈ (total number of carbon atoms) alkylsulfonylalkyl, C₄₋₁₀ (total number of carbon atoms) cycloalkylthioalkyl, C₄₋₁₀ (total number of carbon atoms) cycloalkylsulfinylalkyl, C₄₋₁₀ (total number of carbon atoms) cycloalkylsulfonylalkyl, C₄₋₈ (total number of carbon atoms) trialkylsilylalkyl, C₂₋₈ (total number of carbon atoms) alkoxycarbonylmethylaminocarbonylalkyl, C₂₋₈ (total number of carbon atoms) alkylcarbamoyloxyalkyl, C₂₋₈ (total number of carbon atoms) monoalkylcarbamoylalkyl, C₃₋₈ (total number of carbon atoms) monoalkenylcarbamoylalkyl, C₄₋₈ (total number of carbon atoms) cycloalkylcarbamoylalkyl, C₂₋₈ (total number of carbon atoms) alkoxycarbonylaminoalkyl, C₂₋₈ (total number of carbon atoms) dialkylaminosulfonylalkyl, benzyloxycarbonyl-C₁₋₄ alkyl, benzyloxy-C₁₋₄ alkyl, C₁₋₂ alkylthioaryl, C₁₋₂ alkylsulfinylaryl, C₁₋₂ alkylsulfonylaryl, C₁₋₂ alkylthioheteroaryl, C₁₋₂ alkylsulfinylheteroaryl, C₁₋₂ alkylsulfonylheteroaryl, C₃₋₈ (total number of carbon atoms) cycloalkylalkyl, 5- or 6-membered heterocyclyl or 5- or 6-membered heterocyclyl-C₁₋₄ alkyl which may be substituted,
R¹ and R² together with a nitrogen atom to which they are attached may form a saturated 5- or 6-membered heterocyclic group constituted by a combination of a carbon atom, an oxygen atom and a sulfur atom,
R² and R³ represent a hydrogen atom, or C₁₋₆ alkyl, C₂₋₆ alkenyl, C₂₋₆ alkynyl, C₂₋₈ (total number of carbon atoms) alkoxyalkyl or C₂₋₈ (total number of carbon atoms) alkylthioalkyl which may be substituted,
   W⁵ represents a nitrogen atom, C—Y¹ or a group C-(A)r-Q,
   W⁶ represents a nitrogen atom, C—Y² or a group C-(A)r-Q,
   W⁷ represents a nitrogen atom, C—Y³ or a group C-(A)r-Q,
   W⁸ represents a nitrogen atom, C—Y⁴ or a group C-(A)r-Q,
   W⁹ represents a nitrogen atom, C—Y⁵ or a group C-(A)r-Q,
   under the proviso that at least one of W⁵ to W⁹ necessarily represents C-(A)r-Q,
X¹, X², X³ and X⁴, which may be identical or different, represent a hydrogen atom, nitro, formyl, amino, cyano, halogen, carbamoyl, or C₁₋₆ alkyl, C₁₋₆ haloalkyl, C₁₋₆ alkylcarbonyl, C₁₋₆ alkoxycarbonyl, C₁₋₆ acylamino, C₁₋₆ alkoxy, C₁₋₆ haloalkoxy, C₁₋₆ alkylthio, C₁₋₆ alkylsulfinyl, C₁₋₆ alkylsulfonyl, C₁₋₆ haloalkylthio, C₁₋₆ haloalkylsulfinyl, C₁₋₆ haloalkylsulfonyl, C₃₋₈ cycloalkylthio, C₃₋₈ cycloalkylsulfinyl, C₃₋₈ cycloalkylsulfonyl, C₄₋₁₀ (total number of carbon atoms) cycloalkylalkylthio, C₄₋₁₀ (total number of carbon atoms) cycloalkylalkylsulfinyl, C₄₋₁₀ (total number of carbon atoms) cycloalkylalkylalkylsulfonyl, C₁₋₆ alkylsulfonyloxy, C₁₋₆ haloalkylsulfonyloxy, C₁₋₆ monoalkylaminosulfonyl, C₂₋₈ (total number of carbon atoms) dialkylaminosulfonyl, C₁₋₆ monoalkylamino, C₂₋₈ (total number of carbon atoms) dialkylamino, C₁₋₆ monoalkylcarbamoyl, C₂₋₈ (total number of carbon atoms) dialkylcarbamoyl, C₂₋₆ alkenyl, C₂₋₆ alkenyloxy, C₂₋₆ alkynyl, C₂₋₆ alkynyloxy, C₃₋₈ cycloalkyl, C₃₋₈ cycloalkyloxy which may be substituted, or aryl, aryloxy, arylthio, aryl C₁₋₄ alkyl, aryl C₁₋₄ alkoxy which may be substituted, aryl C₁₋₄ alkylthio, heteroaryl, heterocyclyl, heterocyclyloxy, heterocyclylthio or heterocyclyl C₁₋₄ alkoxy which may be substituted by nitro, formyl, cyano, halogen, and C₁₋₄ alkyl, C₁₋₄ haloalkyl, C₁₋₄ alkylcarbonyl, C₁₋₄ alkoxycarbonyl, C₁₋₄ alkoxy, C₁₋₄ haloalkoxy, C₁₋₄ alkylthio or C₁₋₄ haloalkylthio which may be substituted, preferably X¹, X², X³ and X⁴, which may be identical or different, represent a hydrogen atom, nitro, formyl, amino, cyano, halogen, carbamoyl, or C₁₋₄ alkyl, C₁₋₄ haloalkyl, C₁₋₄ alkylcarbonyl, C₁₋₄ alkoxycarbonyl, C₁₋₄ acylamino, C₁₋₄ alkoxy, C₁₋₄ haloalkoxy, C₁₋₄ alkylthio, C₁₋₄ alkylsulfinyl, C₁₋₄ alkylsulfonyl, C₁₋₄ haloalkylthio, C₁₋₄ haloalkylsulfinyl, C₁₋₄ haloalkylsulfonyl, C₃₋₆ cycloalkylthio, C₃₋₆ cycloalkylsulfinyl, C₃₋₆ cycloalkylsulfonyl, C₄₋₈ (total number of carbon atoms) cycloalkylalkylthio, C₄₋₈ (total number of carbon atoms) cycloalkylalkylsulfinyl, C₄₋₈ (total number of carbon atoms) cycloalkylalkylsulfonyl, C₁₋₄ alkylsulfonyloxy, C₁₋₄ haloalkylsulfonyloxy, C₁₋₄ monoalkylaminosulfonyl, C₂₋₆ (total number of carbon atoms) dialkylaminosulfonyl, C₁₋₄ monoalkylamino, C₂₋₆ (total number of carbon atoms) dialkylamino, C₁₋₄ monoalkylcarbamoyl, C₂₋₆ (total number of carbon atoms) dialkylcarbamoyl, C₂₋₄ alkenyl, C₂₋₄ alkenyloxy, C₂₋₄ alkynyl, C₂₋₄ alkynyloxy, C₃₋₆ cycloalkyl, C₃₋₆ cycloalkyloxy which may be substituted, or aryl, aryloxy, arylthio, aryl C₁₋₂ alkyl, aryl C₁₋₂ alkoxy, aryl C₁₋₂ alkylthio, heteroaryl, heterocyclyl, heterocyclyloxy, heterocyclylthio or heterocyclyl C₁₋₂ alkoxy which may be substituted by nitro, formyl, cyano, halogen, C₁₋₂ alkyl, C₁₋₂ haloalkyl, C₁₋₂ alkylcarbonyl, C₁₋₂ alkoxycarbonyl, C₁₋₂ alkoxy, C₁₋₂ haloalkoxy, C₁₋₂ alkylthio or C₁₋₂ haloalkylthio,
   or wherein X¹ together with X², or X² together with X³, or X³ together with X⁴ may form a 5- or 6-membered carbon ring or a heterocyclic group constituted by a combination of oxygen, sulfur, nitrogen which may be substituted by C₁₋₂ alkyl, and carbon which may be substituted by halogen, preferably X¹ together with X², or X² together with X³, or X³ together with X⁴ form together one of the following carbon ring or a heterocyclic group

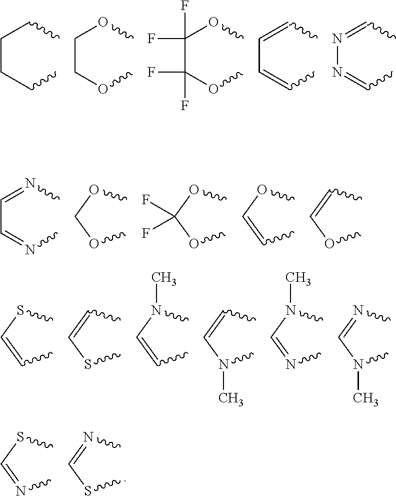

Y¹, Y², Y³, Y⁴ and Y⁵, which may be identical or different, represent a hydrogen atom, nitro, formyl, carbamoyl, amino, cyano, halogen, or C₁₋₆ alkyl, C₁₋₆ haloalkyl, C₁₋₆ alkylcarbonyl, C₁₋₆ alkoxycarbonyl, C₁₋₆ acylamino, C₁₋₆ alkoxy, C₁₋₆ haloalkoxy, C₂₋₈ (total number of carbon atoms) alkoxycarbonylalkyl, C₁₋₆ alkylthio, C₁₋₆ alkylsulfinyl, C₁₋₆ alkylsulfonyl, C₁₋₆ haloalkylthio, C₂₋₈ (total number of carbon atoms) alkylthioalkyl, C₁₋₆ monoalkylamino, C₂₋₈ (total number of carbon atoms) dialkylamino, C₁₋₆ monoalkylcarbamoyl, C₂₋₈ (total number of carbon atoms) dialkylcarbamoyl, C₂₋₆ alkenyl, C₂₋₆ alkenyloxy, C₂₋₆ alkynyl, C₂₋₆ alkynyloxy, C₃₋₈ cycloalkyl, C₃₋₈ cycloalkyloxy, C₄₋₉ (total number of carbon atoms) cycloalkylalkyl, C₄₋₉ (total number of carbon atoms) cycloalkylalkoxy which may be substituted, or aryl, aryloxy, arylthio, aryl C₁₋₄ alkyl, aryl C₁₋₄ alkoxy, aryl C₁₋₄ alkylthio, heteroaryl, heterocyclyl, heterocyclyloxy, heterocyclylthio or heterocyclyl C₁₋₄ alkoxy which may be substituted by at least one selected from the group consisting of formyl, cyano, halogen, C₁₋₄ alkyl, C₁₋₄ haloalkyl, C₁₋₄ alkylcarbonyl, C₁₋₄ alkoxycarbonyl, C₁₋₄ alkoxy, C₁₋₄ haloalkoxy, C₁₋₄ alkylthio and C₁₋₄ haloalkylthio, preferably Y′, Y², Y³, Y⁴ and Y⁵, which may be identical or different, represent a hydrogen atom, nitro, formyl, carbamoyl, amino, cyano, halogen, or C₁₋₄ alkyl, C₁₋₄ haloalkyl, C₁₋₄ alkylcarbonyl, C₁₋₄ alkoxycarbonyl, C₁₋₄ acylamino, C₁₋₄ alkoxy, C₁₋₄ haloalkoxy, C₂₋₆ (total number of carbon atoms) alkoxycarbonylalkyl, C₁₋₄ alkylthio, C₁₋₄ alkylsulfinyl, C₁₋₄ alkylsulfonyl, C₁₋₄ haloalkylthio, C₂₋₆ (total number of carbon atoms) alkylthioalkyl, C₁₋₄ monoalkylamino, C₂₋₆ (total number of carbon atoms) dialkylamino, C₁₋₄ monoalkylcarbamoyl, C₂₋₆ (total number of carbon atoms) dialkylcarbamoyl, C₂₋₄ alkenyl, C₂₋₄ alkenyloxy, C₂₋₄ alkynyl, C₂₋₄ alkynyloxy, C₃₋₆ cycloalkyl, C₃₋₆ cycloalkyloxy, C₄₋₇ (total number of carbon atoms) cycloalkylalkyl, C₄₋₇ (total number of carbon atoms) cycloalkylalkoxy which may be substituted, or aryl, aryloxy, arylthio, aryl C₁₋₂ alkyl, aryl C₁₋₂ alkoxy, aryl C₁₋₂ alkylthio, heteroaryl, heterocyclyl, heterocyclyloxy, heterocyclylthio or heterocyclyl C₁₋₂ alkoxy which may be substituted by at least one selected from the group consisting of formyl, cyano, halogen, C₁₋₂ alkyl, C₁₋₂ haloalkyl, C₁₋₂ alkylcarbonyl, C₁₋₆ alkoxycarbonyl, C₁₋₂ alkoxy, C₁₋₂ haloalkoxy, C₁₋₂ alkylthio and C₁₋₂ haloalkylthio,
   Q is selected from the group consisting of Q1 to Q67, preferably is selected from the group consting of Q1, Q8, Q9, Q11, Q12, Q18, Q20, Q25, Q34, Q35, Q36, Q37, Q38, Q39, Q40, Q41, Q43, Q47, Q48, Q51, Q57 and Q63.

Further preferred are compounds of formula (I) as defined before, wherein

R¹ and R² together with the nitrogen atom to which they are attached forms one of the following 5- or 6-membered groups:

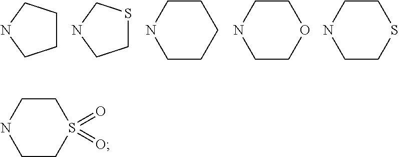

or
R² and R³ represent a hydrogen atom, or C₁₋₄ alkyl, C₂₋₄ alkenyl, C₂₋₄ alkynyl, C₂₋₆ (total number of carbon atoms) alkoxyalkyl or C₂₋₆ (total number of carbon atoms) alkylthioalkyl which may be substituted, and
Q represents a heterocyclic group of Q1, Q8, Q9, Q11, Q12, Q18, Q20, Q25, Q34, Q35, Q36, Q37, Q38, Q39, Q40, Q41, Q43, Q47, Q48, Q51, Q57 or Q63 which are as defined herein, and

The compounds according to the present invention exhibit a potent insecticidal action.

In the present specification,

the term “alkyl” used either alone or combined with other terms such as “aminoalkyl” or “haloalkyl” includes straight-chained or branched alkyl containing up to 12 carbon atoms, such as methyl, ethyl, n- or iso-propyl; n-, iso-, secondary- or tertiary-butyl; n-pentyl, n-hexyl, n-heptyl, n-octyl, n-nonyl, n-decyl, n-undecyl or n-dodecyl, and preferably represents alkyl having 1 to 6 carbon atoms.

The term “acylamino” includes groups like alkylcarbonylamino, cycloalkylcarbonylamino or benzoylamino.

The term “halogen” or “halo” used either alone or combined with other terms such as “haloalkyl” includes fluorine, chlorine, bromine or iodine.

The term “cycloalkyl” used either alone or combined with other terms preferably stands for cycloalkyl groups having 3 to 8 carbon atoms, such as cyclopropyl, cyclobutyl, cyclopentyl, cyclohexyl, cycloheptyl or cyclooctyl, and preferably represents cycloalkyl having 3 to 7 carbon atoms.

The term “alkenyl” used either alone or combined with other terms preferably stands for alkenyl having 2 to 6 or 2 to 5 carbon atoms. Examples include vinyl, allyl, 1-propenyl, 1-, 2-, or 3-butenyl or 1-pentenyl and 1-hexenyl. More preferred it stands for alkenyl having 2 to 4 carbon atoms.

The term “alkynyl” used either alone or combined with other terms preferably stands for alkynyl having 2 to 6 or 2 to 5 carbon atoms. Examples include ethynyl, propargyl, 1-propynyl, but-3-ynyl or pent-4-ynyl. More preferred it stands for alkynyl having 2 to 4 carbon atoms.

The term “heterocyclic group” used either alone or combined with other terms preferably stands for a 5- or 6-membered heterocyclic group containing at least one of N, O and S as a heteroatom. Typically a heterocyclic group contains no more than 4 nitrogens, 2 oxygens and 2 sulfur atoms. The cyclic group, the ring, can be saturated, unsaturated or partially saturated. If not mentioned otherwise, than a heterocyclic group can be can be attached through any available carbon or heteroatom. The term additionally includes fused heterocyclic group which may then be benzo-condensed. Heterocyclic group include for example pyrrolyl, pyrazolyl, isoxazolyl, imidazolyl, oxazolyl, triazolyl, oxadiazolyl, tetrazolyl, pyrimidinyl, triazinyl and dihydrotriazolyl.
A compound of the formula (I) of the invention can be obtained according to preparation processes (a) to (O) shown below.

Preparation process (a) for compounds of formula (I) wherein R² represents hydrogen and which comprises the following step:

### Reacting a Compound (II):

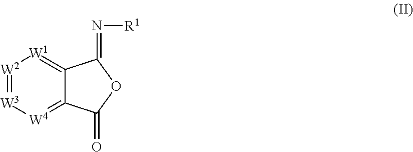 [0041] with a compound (III):

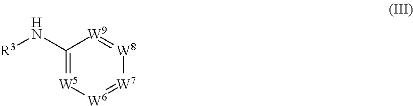

whereas R¹, W¹, W², W³ and W⁴, R³, W⁵, W⁶, W⁷, W⁸ and W⁹ are as defined herein.

Preparation process (b) for compounds of formula (I), wherein R² and R³ represent hydrogen and which comprises the following step:

### Reacting a Compound (Iv)

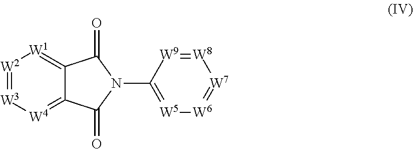

with a compound of formula (V)

H₂N—R¹ (V)

whereas W¹, W², W³, W⁴, W⁵, W⁶, W⁷, W⁸ and W⁹ and R¹ are as defined herein.

Preparation process (c) for compounds of formula (I) comprising the following step:

### Reacting a Compound (VI)

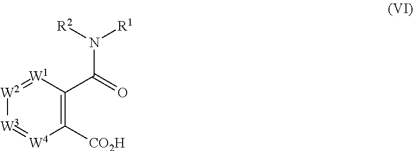

wherein R¹, R², W¹, W², W³ and W⁴ are as defined herein with the compound of formula (III).

Preparation process (d) for compounds of formula (I), wherein W¹ stands for C—CN comprising the following step:

### Reacting a Compound of the Formula (IA)

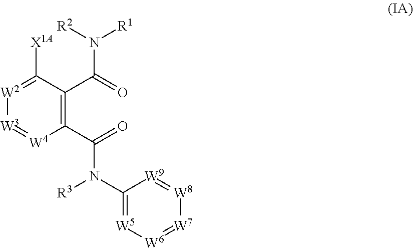

wherein X^{1A} represents bromine or iodine, and W², W³, W⁴, R¹, R², R³, W⁵, W⁶, W⁷, W⁸ and W⁹ are as defined herein with cuprous cyanide or zinc cyanide.

Preparation process (e) for compounds of formula (I), wherein W¹ represents C-T, and T represents optionally substituted phenyl comprising the following step of reacting a compound of the formula (IA) with a compound of the formula (VII)

T-B(OH)₂ (VII)

Preparation process (f) for compounds of formula (I), wherein W⁷ represents C—CH₂-Q38 comprising reacting a compound of the following formula (VIII):

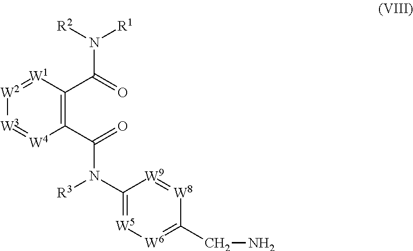

wherein R¹, R², R³, W¹, W², W³, W⁴, W⁵, W⁶ W⁷, W⁸ and W⁹ are as defined herein
with a compound of the following formula (IX):

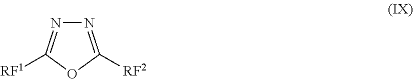

wherein RF¹ and RF² stand for C₁₋₄ fluoroalkyl.

Preparation process (g) for compounds of formula (I), wherein R¹ represents carboxyalkyl, and R² represents a hydrogen atom which comprises the following step:

### Debenzylating a Compound of Formula (IB)

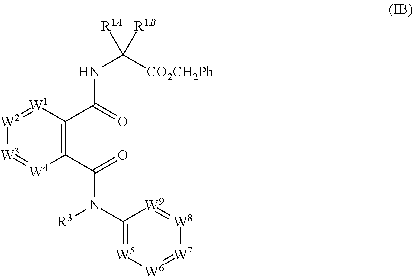 [0053] wherein R³, W¹, W², W³, W⁴, W⁵, W⁶, W⁷, W⁸ and W⁹ are as defined herein, R^{1A} and R^{1B} represent a hydrogen or C₁₋₄ alkyl, and Ph represents phenyl
   with boron tribromide or by catalytic hydrogen reduction.

Preparation process (h) for compounds of formula (I), wherein R¹ represents alkoxycarbonylmethylaminocarbonylalkyl, alkylcarbamoylalkyl or cycloalkylcarbamoylalkyl and R² represents a hydrogen atom and which comprises the following step:

### Subjecting a Compound of Formula (IC)

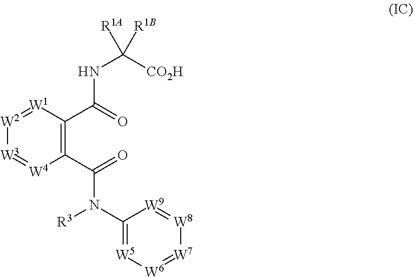

wherein R^{1A}, R^{1B}, R³, W¹, W², W³, W⁴, W⁵, W⁶, W⁷, W⁸ and W⁹ are as defined herein, and a compound of the following formula:

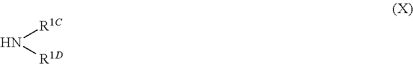

wherein R^{1C} and R^{1D} represent a hydrogen atom, a C₁₋₆ alkyl group, a C₃₋₆ cycloalkyl group or a C₁₋₄ alkoxycarbonyl C₁₋₄ alkyl to a condensation reaction.

Preparation process (i) for compounds of formula (I), wherein R¹ represents hydroxyalkyl, and R² represents a hydrogen atom which comprises subjecting the compound represented by the following formula (ID):

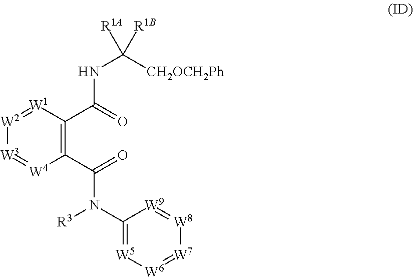

wherein R^{1A}, R^{1B}, R³, W¹, W², W³, W⁴, W⁵, W⁶, W⁷, W⁸ and W⁹ are as defined herein
to a debenzylation reaction with boron tribromide or by catalytic hydrogen reduction.

Preparation process (j) for compounds of formula (I), wherein R¹ represents formylalkyl, and R² represents a hydrogen atom which comprises reacting a compound of the following formula (IE)

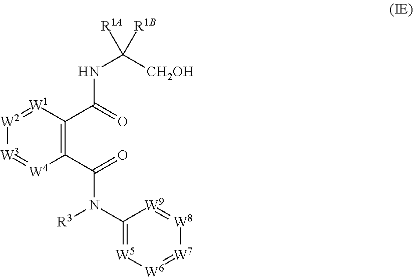

wherein R^{1A}, R^{1B}, R³, W¹, W², W³, W⁴, W⁵, W⁶, W⁷, W⁸ and W⁹ are as defined herein with an oxidizing agent.

Preparation process (k) for compounds of formula (I), wherein R¹ represents hydroxyiminoalkyl or alkoxyiminoalkyl, and R² represents a hydrogen atom comprising reacting a compound represented by the following formula (IF)

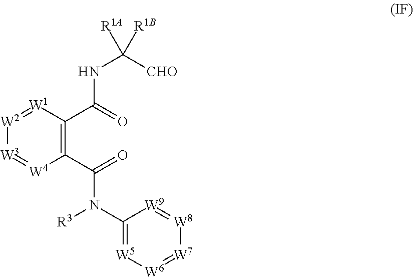

wherein R^{1A}, R^{1B}, R³, W¹, W², W³, W⁴, W⁵, W⁶, W⁷, W⁸ and W⁹ are as defined herein, with a compound represented by the following formula:

NH₂OR^{1E} (XI)

wherein R^{1E} represents a hydrogen atom or a C₁₋₄ alkyl group.

Preparation process (l) for compounds of formula (I), wherein R¹ represents alkylsulfinylalkyl, alkylsulfonylalkyl, cycloalkylsulfinyl-alkyl, cycloalkylsulfonylalkyl, alkylsulfinylaryl, alkylsulfonylaryl, alkylsulfinylheteroaryl or alkylsulfonyl-heteroaryl comprising reacting a compound of the following formula (IG):

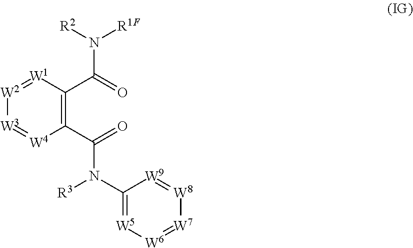

wherein R^{1F} represents alkylthioalkyl, cycloalkyl-thioalkyl, alkylthioaryl or alkylthioheteroaryl, and R³, W¹, W², W³, W⁴, W⁵, W⁶, W⁷, W⁸ and W⁹ are as defined herein, with an oxidizing agent.

Preparation process (m) for compounds of formula (I), wherein W⁷ represents CH(OH)CH₂Q comprising reacting a compound of the following formula (II):

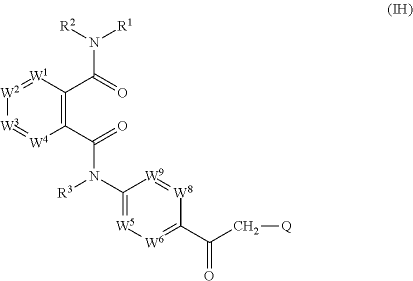

wherein R¹, R², R³, W¹, W², W³, W⁴, W⁵, W⁶, W⁸, W⁹ and Q are as defined herein with a reducing agent.

Preparation process (n) for compounds of formula (I), wherein W⁷ stand for C-(A)r-Q, wherein A represents CH₂, r=1 and Q is Q63, and R⁵ represents alkylcarbonyl, haloalkylcarbonyl or alkoxycarbonyl, comprising reacting a compound of formula (IJ):

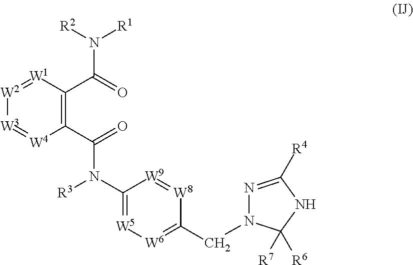

wherein R¹, R², R³, R⁴, R⁶, R⁷, W¹, W², W³, W⁴, W⁵, W⁶, W⁸, and W⁹ are as defined herein, with a compound of formula (XII) or formula OCR

R^{5A}—COCl (XII)

(R^{5A}—CO)₂O (XIII)
wherein R^{5A} represents C₁₋₄ alkyl, C₁₋₄ haloalkyl or C₁₋₄ alkoxy.

Preparation process (o) for compounds of formula (I), wherein W⁷ represents C—CH₂-Q, and one of the substituent R⁴ to R⁷ on Q represents hydroxyiminoalkyl, alkoxyiminoalkyl, hydroxyiminohaloalkyl or alkoxyiminohaloalkyl comprising reacting a compound represented by the following formula (IK)

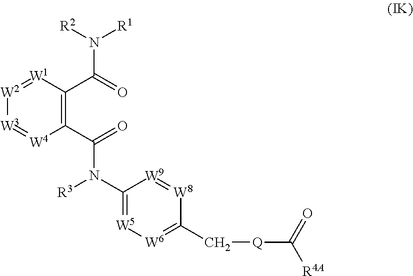

wherein R^{4A} represents C₁₋₄ alkyl or C₁₋₄ haloalkyl, and a group C(═O)R^{4A} is bonded to a carbon atom of Q, and R¹, R², R³, W¹, W², W³, W⁴, W⁵, W⁶, W⁸ and W⁹ are as defined herein, with the compound represented by the formula (XI).

Preparation process (a) may be represented by the following reaction scheme, when, for example, 3-(isopropylimino)-2-benzofuran-1(3H)-one and 4-{[3,5-bis(trifluoromethyl)-1H-pyrazol-1-yl]methyl}aniline are used as starting materials.

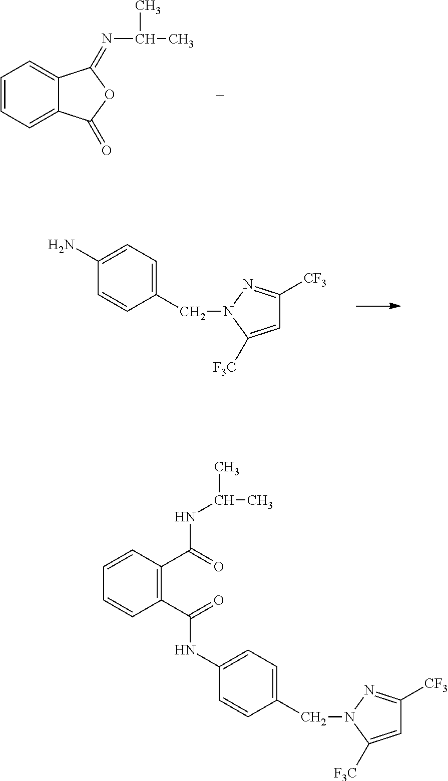

Preparation process (b) may be represented by the following reaction scheme, when, for example, 2-(4-{[3,5-bis(trifluoromethyl)-1H-pyrazol-1-yl]methyl}-2-methylphenyl)-1H-isoindole-1,3(2H)-dione and isopropylamine are used as starting materials.

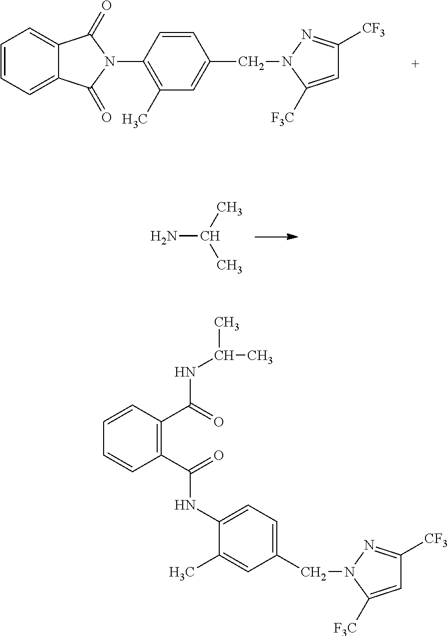

Preparation process (c) may be represented by the following reaction scheme, when, for example, 3-chloro-2-(diethylcarbamoyl)benzoic acid and 4-{[3,5-bis(trifluoro-methyl)-1H-pyrazol-1-yl]methyl}-2-methylaniline are used as starting materials.

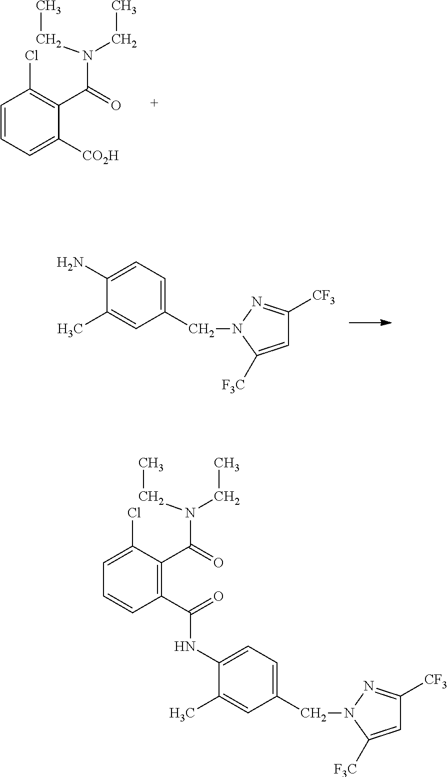

Preparation process (d) may be represented by the following reaction scheme, when, for example, N′-(4-{([3,5-bis(trifluoromethyl)-1H-pyrazol-1-yl]methyl}-2-methylphenyl)-3-iodo-N²-[(1S)-1-methyl-2-(methylthio)ethyl]-1,2-benzenedicarboxamide and cuprous cyanide are used as starting materials.

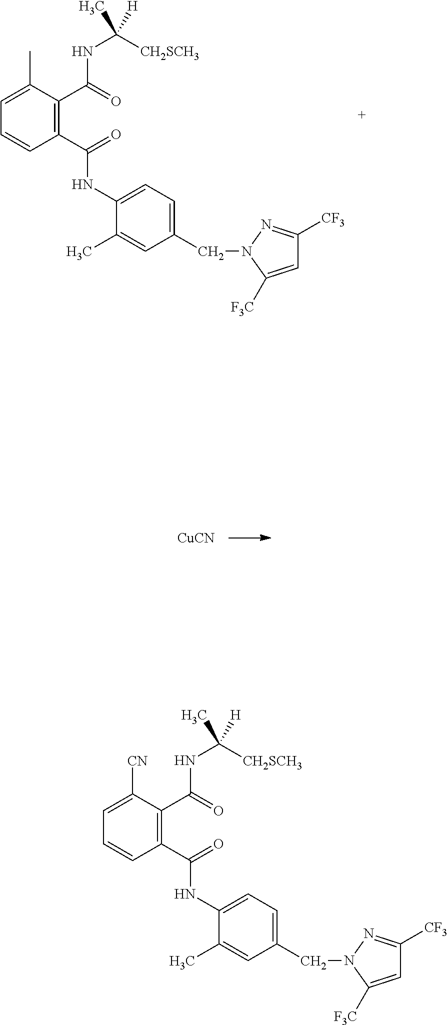

Preparation process (e) may be represented by the following reaction scheme, when for example, 3-bromo-N²-[(1S)-1-methyl-2-(methylsulfonyl)ethyl]-N¹-(2-methyl-4-{[3-(pentafluoroethyl)-5-(trifluoromethyl)-1H-1,2,4-triazol-1-yl]methyl}phenyl)-1,2-benzenedicarboxamide and 3,5-bis(trifluoromethyl)phenylboronic acid are used as starting materials.

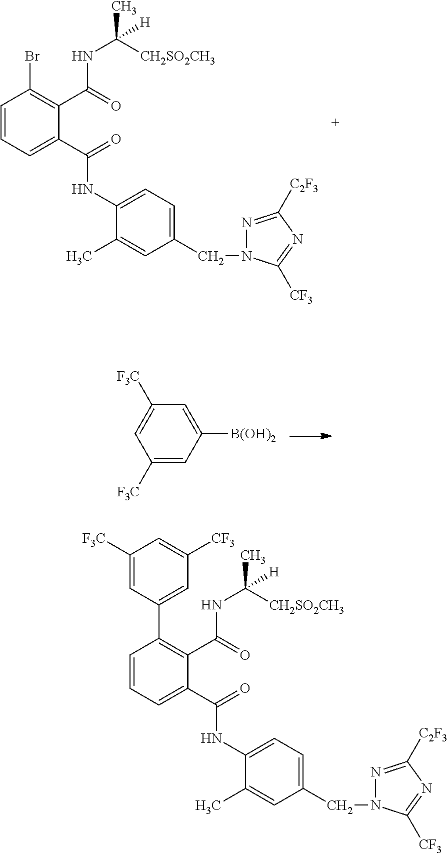

Preparation process (f) may be represented by the following reaction scheme when, for example, N¹-[4-(aminomethyl)-2-methylphenyl]-3-chloro-N²-[(1S)-1-methyl-2-(methylthio)ethyl]-1,2-benzenedicarboxamide and 2,5-bis(trifluoromethyl)-1,3,4-oxadiazole are used as starting materials.

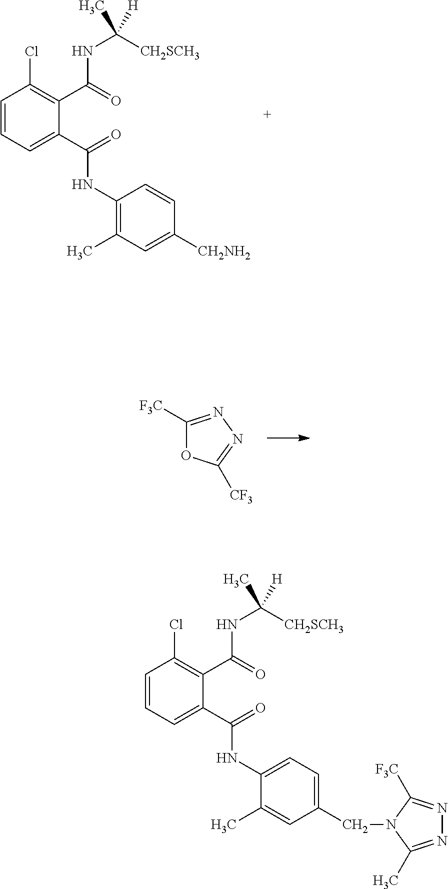

Preparation process (g) may be represented by the following reaction scheme when, for example, benzyl N-({2-[(4-{[3,5-bis(trifluoromethyl)-1H-pyrazol-1-yl]methyl}-2-methylphenyl)carbamoyl]-6-chlorobenzoyl}-2-methyl alaninate and boron tribromide are used as starting materials.

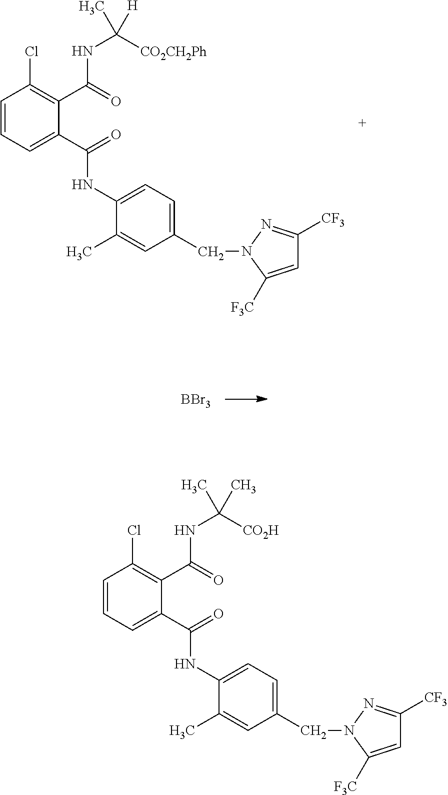

Preparation process (h) may be represented by the following reaction scheme when, for example, N-({[2-[(4-{[3,5-bis(trifluoromethyl)-1H-pyrazol-1-yl]methyl}-2-methylphenyl)carbamoyl]-6-chlorobenzoyl]-2-methylalanine and glycine methyl ester hydrochloride are used as starting materials.

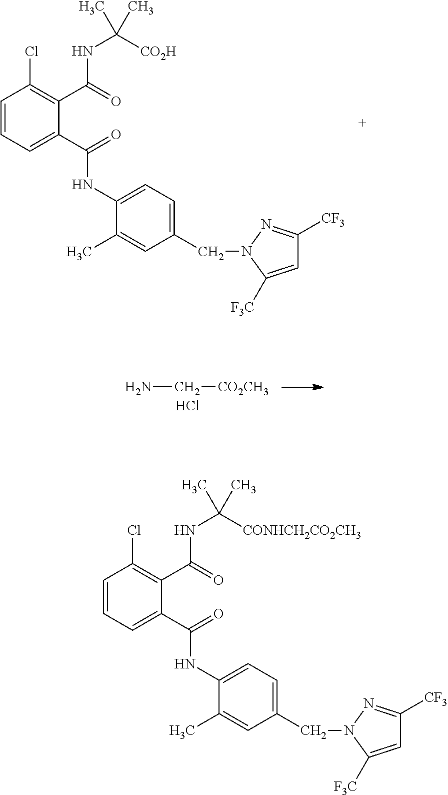

Preparation process (i) may be represented by the following reaction scheme, when, for example, N²-[2-(benzyloxy)-1,1-dimethylethyl]-3-chloro-N¹-(2-methyl-4-{[3-(pentafluoroethyl)-5-(trifluoromethyl)-1H-1,2,4-triazol-1-yl]methyl}phenyl)-1,2-benzenedicarboxamide and boron tribromide used as starting materials.

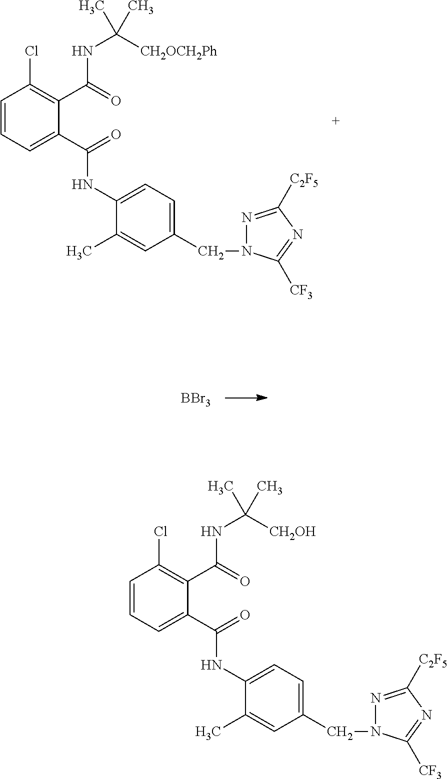

Preparation process (j) may be represented by the following reaction scheme when, for example, 3-chloro-N²-(2-hydroxy-1,1-dimethylethyl)-N′-(2-methyl-4-{[3-(pentafluoroethyl)-5-(trifluoromethyl)-1H-1,2,4-triazol-1-yl]methyl}phenyl)-1,2-benzenedicarboxamide, dimethyl sulfoxide and oxalyl chloride are used as starting materials.

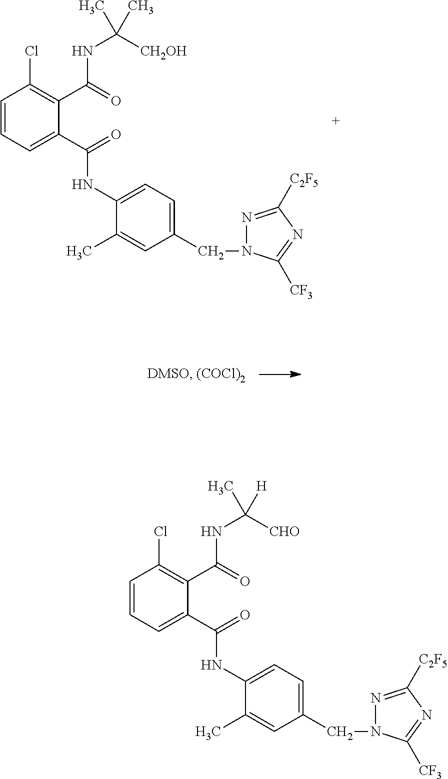

Preparation process (k) may be represented by the following reaction scheme when, for example, 3-chloro-N²-(1,1-dimethyl-2-oxoethyl)-N¹-(2-methyl-4-{[3-(pentafluoroethyl)-5-(trifluoromethyl)-1H-1,2,4-triazol-1-yl]methyl}phenyl)-1,2-benzenedicarboxamide and hydroxylamine hydrochloride are used as starting materials.

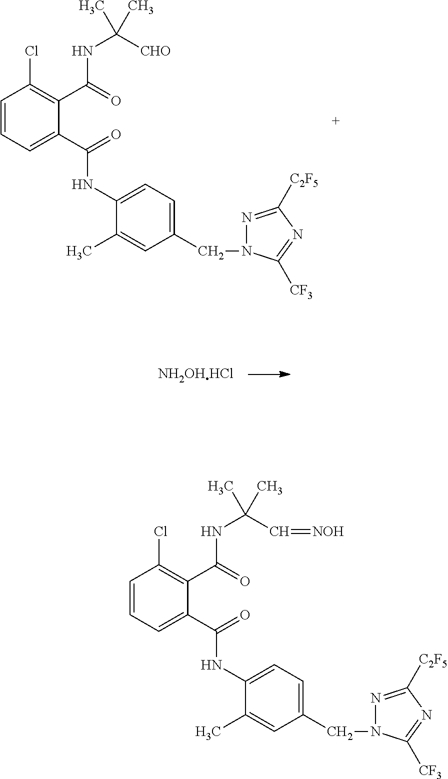

Preparation process (l) may be represented by the following reaction scheme when, for example, N¹-(4-{[3,4-bis(pentafluoroethyl)-1H-pyrazol-1-yl]methyl}-2-methylphenyl)-3-bromo-N²-[(1S)-1-methyl-2-(methylthio)ethyl]-1,2-benzenedicarboxamide and hydrogen peroxide are used as starting materials.

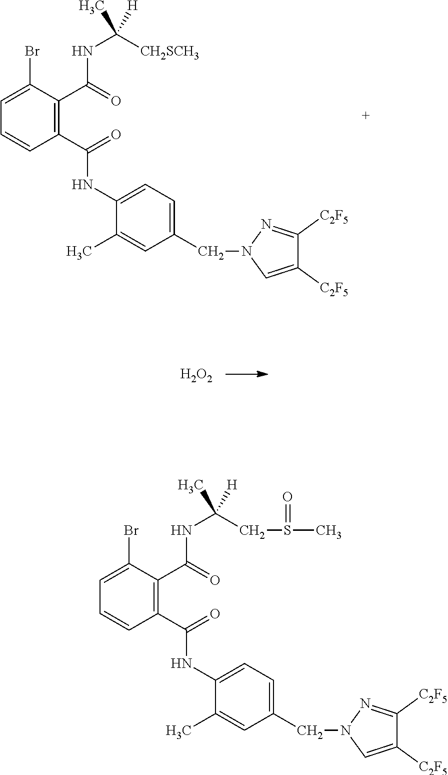

Preparation process (m) may be represented by the following reaction scheme when, for example, N¹-(4-{[3,5-bis(trifluoromethyl)-1H-pyrazol-1-yl]acetyl}-2-methylphenyl)-3-chloro-N²-[(1S)-1-methyl-2-(methylthio)ethyl]-1,2-benzenedicarboxamide and sodium borohydride are used as starting materials.

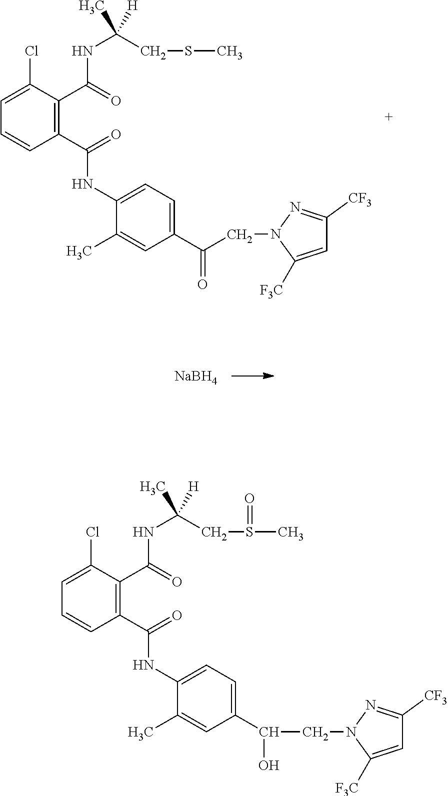

Preparation process (n) may be represented by the following reaction scheme when, for example, 3-chloro-N¹-(2-methyl-4-{[3-(pentafluoroethyl)-5-(trifluoromethyl)-4,5-dihydro-1H-1,2,4-triazol-1-yl]methyl}phenyl)-N²-[(1S)-1-methyl-2-(methylthio)ethyl]-1,2-benzenedicarboxamide and acetic anhydride are used as starting materials.

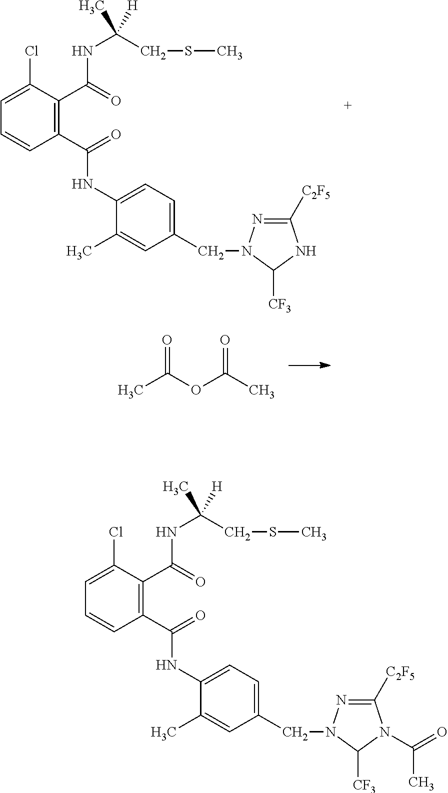

Preparation process (o) may be represented by the following reaction scheme when, for example, 3-iodo-N¹-(2-methyl-4-{[4-(trifluoroacetyl)-3-(trifluoromethyl)-1H-pyrazol-1-yl]methyl}phenyl)-N²-[(1S)-1-methyl-2-(methylthio)ethyl]-1,2-benzenedicarboxamide and hydroxylamine hydrochloride are used as starting materials.

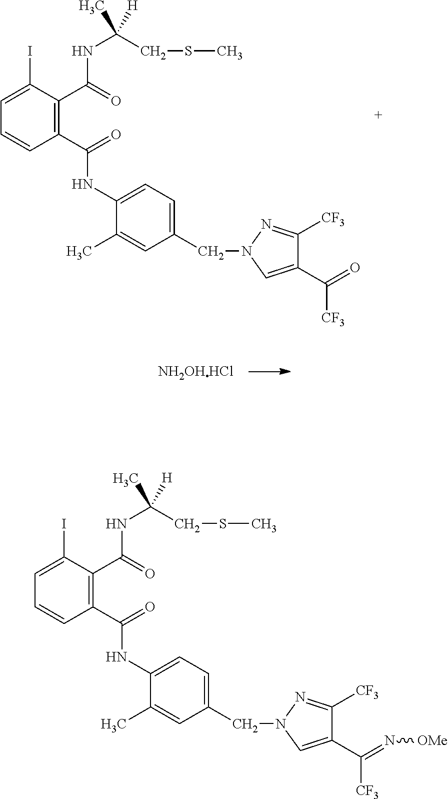

The compounds of the formula (II) used as a starting material in preparation process (a) include known compounds and may be synthesized in accordance with the methods described in Japanese Patent Application Laid-Open No. 11-240857, Japanese Patent Application Laid-Open No. 2001-131141 and WO 2006/024402.

Specific examples of the compounds include the following compounds: 3-(isopropylimino)-2-benzofuran-1(3H)-one, 4-fluoro-3-(isopropylimino)-2-benzofuran-1-(3H)-one, 4-chloro-3-(isopropylimino)-2-benzofuran-1(3H)-one, 4-bromo-3-(isopropylimino)-2-benzofuran-1(3H)-one, 4-iodo-3-(isopropylimino)-2-benzofuran-1(3H)-one, 3-{[1-methyl-2-(methylthio)ethyl]imino}-2-benzofuran-1(3H)-one, 4-fluoro-3-{[1-methyl-2-(methylthio)ethyl]imino}-2-benzofuran-1(3H)-one, 4-chloro-3-{[1-methyl-2-(methylthio)ethyl]imino}-2-benzofuran-1(3H)-one, 4-bromo-3-{[1-methyl-2-(methylthio)ethyl]imino}-2-benzofuran-1(3H)-one, 4-iodo-3-{[1-methyl-2-(methylthio)ethyl]imino}-2-benzofuran-1(3H)-one, 4-fluoro-3-{[(1S)-1-methyl-2-(methylthio)ethyl]imino}-2-benzofuran-1(3H)-one, 4-chloro-3-{[(1S)-1-methyl-2-(methylthio)ethyl]imino}-2-benzofuran-1(3H)-one, 7-chloro-3-{[(1S)-1-methyl-2-(methylthio)ethyl]imino}-2-benzofuran-1(3H)-one, 4-bromo-3-{[(1S)-1-methyl-2-(methylthio)ethyl]imino}-2-benzofuran-1(3H)-one, 4-iodo-3-{[(1S)-1-methyl-2-(methylthio)ethyl]imino}-2-benzofuran-1(3H)-one, 4-methyl-3-{[(1S)-1-methyl-2-(methylthio)ethyl]imino}-2-benzofuran-1(3H)-one, 4,5-dichloro-3-{[(1S)-1-methyl-2-(methylthio)ethyl]imino}-2-benzofuran-1(3H)-one, 3-{[(1S)-1-methyl-2-(methylthio)ethyl]imino}-4-(trifluoromethyl)-2-benzofuran-1(3H)-one, and 5-chloro-3-{[(1S)-1-methyl-2-(methylthio)ethyl]imino}-2-benzofuran-1(3H)-one.

The compounds of the formula (III) used as a starting material in preparation process (a) include known compounds described in Japanese Patent Application Laid-Open No. 11-240832, Japanese Patent Application Laid-Open No. 2004-277333, Japanese Patent Application Laid-Open No. 2006-76990 or WO 2006/053643. Specific examples thereof include the following compounds: 4-{[3,5-bis(trifluoromethyl)-1H-pyrazol-1-yl]methyl}aniline, 1-(4-amino-3-methylbenzyl)-4-[3-(trifluoromethyl)phenyl]-1,4-dihydro-5H-tetrazol-5-one, 1-(4-amino-3-methylbenzyl)-4-[4-(trifluoromethyl)phenyl]-1,4-dihydro-5H-tetrazol-5-one, 1-(4-amino-3-methylbenzyl)-4-[3,5-bis(trifluoromethyl)phenyl]-1,4-dihydro-5H-tetrazol-5-one, 6-{[3,5-bis(trifluoromethyl)-1H-pyrazol-1-yl]methyl}-2-methylpyridine-3-amine, 4-{[3,5-bis(trifluoromethyl)-1H-pyrazol-1-yl]methyl}-2-methylaniline, and 4-{1-[3,5-bis(trifluoromethyl)-1H-pyrazol-1-yl]methyl}-2-methylaniline.

The compound of the formula (III) may also be synthesized according to a method shown below.

When R³ is hydrogen, a compound of the formula (IIIc) may be obtained by reducing the corresponding nitro compound of the formula (XIV) according to the process described in Japanese Patent Application Laid-Open No. 2006-76990 or WO 2006/053643. For example, the compound of the formula (IIIa) may be obtained by reducing the compound of the formula (XIV) according to a process of reduction reaction well known in the field of organic chemistry:

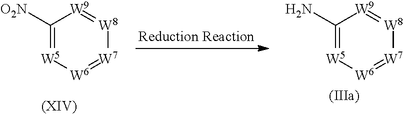

wherein W⁵, W⁶, W⁷, W⁸ and W⁹ have the same meaning as that mentioned herein.

Examples of the reduction process include processes which use metal and are well known in the field of organic chemistry, such as a process in which iron powder is reacted in acetic acid, a process in which zinc powder is reacted under neutral conditions (Organic Syntheses Collective vol. II, p. 447), a process in which tin chloride (II) is reacted under acidic conditions (Organic Syntheses Collective vol. II, p. 254), a process in which titanium trichloride is reacted under neutral conditions, or a catalytic hydrogen reduction.

The process in which iron powder is reacted in acetic acid is carried out in an appropriate diluent. Examples of the diluents used therefor include water, acetic acid and ethyl acetate. The reaction is carried out generally at a temperature from about 0 to 100° C., preferably from room temperature to about 80° C. This reaction is desirably carried out under normal pressure although it may also be carried out under increased pressure or reduced pressure.

When R³ is alkyl, alkoxyalkyl, alkylthioalkyl, alkenylalkyl or alkynylalkyl, the compound of the following formula (IIIb) can be readily synthesized according to a process well known in the field of organic chemistry, for example, by protecting the compound of the formula (IIIa) to form the protected compound of the formula (XV) and then carrying out the deprotection thereof via an alkylated compound of the formula (XVII):

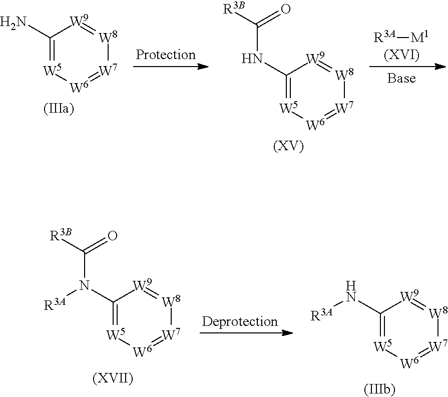

wherein R^{3A} represents C₁₋₄ alkyl, C₂₋₆ (total number of carbon atoms) alkoxyalkyl, C₂₋₆ (total number of carbon atoms) alkylthioalkyl, C₃₋₄ alkenylalkyl or C₃₋₄ alkynylalkyl, R^{3B} represents hydrogen, C₁₋₄ alkoxy or benzyloxy, M¹ represents chloro, bromo or iodo, and W⁵, W⁶, W⁷, W⁸ and W⁹ are as defined herein.

Examples of processes for introducing a protective group in the compound of the formula (IIIa) include a process of introducing a formyl group in accordance with a method described in J. Chem. Soc. vol. 67 (1895), p. 830 and a process of introducing a t-butoxycarbonyl group in accordance with a method described in J. Org. Chem. vol. 65 (2000), pp. 6368-6380.

When the reaction of introducing a protective group is carried out, for example, 1 mole of the compound of the formula (IIIa) may be reacted with 1 mole or slightly excessive mole amount of a protecting agent, such as di-t-butyl bicarbonate, in a diluent, such as toluene, to obtain the compound of the formula (XV).

When the compound of the formula (XV) is alkylated, a process described in J. Org. Chem., vol. 67 (2002), pp. 3949-3952 may be applied.

The alkyl halide represented by the formula (XVI) is a compound well known in the field of organic chemistry, and specific examples thereof include methyl bromide, methyl iodide, ethyl bromide, ethyl iodide, 2-chloroethyl methyl ether, 2-chloroethyl methyl sulfide, aryl bromide and propargyl bromide.

When the alkylation reaction is carried out, for example, 1 mole of the compound of the formula (XV) may be reacted with the compound of the formula (XVI), such as 1 mole or slightly excess mole amount of methyl iodide, in the presence of 1 mole or slightly excess mole amount of a base, such as sodium hydride, in a diluent, such as THF, to obtain the compound of the formula (XVII).

Examples of the process of deprotection of the formula (XVII) include, for example, a process described in Tetrahedron, vol. 57, No. 43 (2001) pp. 9033-9044.

When the reaction is carried out, 1 mole of the compound of the formula (XVII) may be reacted with an excess mole amount (5 mole) of acid, such as trifluoroacetic acid, in a diluent, such as dichloromethane to obtain the compound of the formula (IIIb).

The compound of the formula (IIIc) below which is a compound of the formula (III) wherein W⁵ is CH can be halogenated according to a process described, for example, in J. Org. Chem., vol. 29 (1964), pp. 3390-3396 or J. Org. Chem., vol. 68 (2003), pp. 1843-1851, to obtain the compound represented by the formula (IIId):

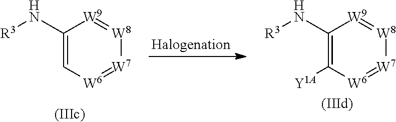

wherein Y^{1A} represents chloro, bromo or iodo, and R³, W⁶, W⁷, W⁸ and W⁹ are as defined herein.

The halogenation reaction may be carried out in an appropriate diluent, and examples of diluents used therefor include aromatic hydrocarbons (optionally chlorinated), such as benzene, chlorobenzene and dichlorobenzene; acid amides, such as dimethylformamide (DMF); alcohols, such as isopropyl alcohol; and acids, such as acetic acid. Examples of halogenating agents include N-chlorosuccinimide (NCS), N-bromosuccinimide (NBS), N-iodosuccinimide (NIS) and iodine monochloride.

The halogenation reaction may be carried out generally at a temperature from about 0 to 150° C., preferably at a temperature from room temperature to about 100° C. The reaction is desirably carried out under normal pressure although it may also be carried out under increased or reduced pressure.

When the reaction is carried out, for example, 1 mole of the compound of the formula (IIIc) may be reacted with 1 mole or slightly excess mole amount of halogenating agent, such as N-chlorosuccinimide, in a diluent such as DMF to obtain the compound of the formula (IIId).

The compound of the formula (IIIc) below, which is a compound of the formula (III) wherein W⁵ is CH, may be alkylthioalkylated according to a process described in J. Amer. Chem. Soc., vol. 96 (1974), pp. 5487-5495 to obtain the compound represented by the formula (IIIe):

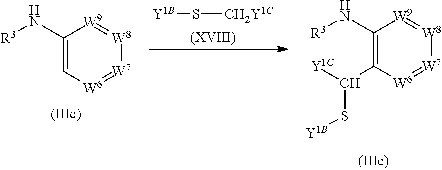

wherein Y^{1B} represents C₁₋₄ alkyl, Y^{1C} represents a hydrogen atom or C₁₋₁₄ alkyl, and R³, W⁶, W⁷, W⁸ and W⁹ are as defined herein.

The alkylthioalkylation reaction may be carried out in an appropriate diluent and examples of diluents used therefor include chlorinated aliphatic hydrocarbons such as methylene chloride.

The alkylthioalkylation reaction may be carried out generally at a temperature from about −78 to 100° C., preferably at a temperature from −60 to 60° C. The reaction is desirably carried out under normal pressure although it may also be carried out under increased or reduced pressure.

When the reaction is carried out, 1 mole of the compound of the formula (Mc) may be reacted with 1 mole or slightly excess mole amount of the compound of the formula (XVIII) and 1 mole or slightly excess mole amount of a halogenating agent, such as N-chlorosuccinimide, in a diluent, such as methylene chloride, and then reacted with a base, such as 1 mole or slightly excess mole amount of triethylamine, to obtain the compound of the formula (IIIe).

Specific examples of compounds of the formula (III) used as a starting material in preparation process (a) include the following compounds: (4-amino-3-methylphenyl)[3-(pentafluoroethyl)-1H-pyrazol-5-yl]methanol, (4-amino-3-methylphenyl)[3-(pentafluoroethyl)-1H-pyrazol-5-yl]methanone, (4-amino-3-methylphenyl)[3,5-bis(trifluoromethyl)-1H-pyrazol-1-yl]acetonitrile, (4-amino-3-methylphenyl) [4-(heptafluoropropyl)-6-(trifluoromethyl)pyrimidin-2-yl]acetonitrile, (4-amino-3-methylphenyl)[4-(pentafluoroethyl)-1,3-thiazol-2-yl]methanone, (4-amino-3-methylphenyl) [4-(pentafluoroethyl)-1,3-thiazol-2-yl]methanone O-methyloxime, (4-amino-3-methylphenyl)[4,6-bis(trifluoromethyl)pyrimidin-2-yl]acetonitrile, (4-amino-3-methylphenyl)[4,6-bis(pentafluoroethyl)pyrimidin-2-yl]acetonitrile, (4-amino-3-methylphenyl)[5-(trifluoromethyl)isoxazol-3-yl]methanone, 1-(4-amino-3-methylphenyl)-2-[3,5-bis(trifluoromethyl)-1H-pyrazol-1-yl]ethanone, 1-(4-amino-3-methylbenzyl)-3-(trifluoromethyl)-1H-1,2,4-triazol-5-amine, 1-(4-amino-3-methylbenzyl)-3-(trifluoromethyl)-1H-pyrazole-4-carbonitrile, 1-(4-amino-3-methylbenzyl)-3-(trifluoromethyl)-N-(1,1,1-trifluoropropan-2-yl)-1H-pyrazole-4-carboxamide, 1-(4-amino-3-methylbenzyl)-3-(pentafluoro ethyl)-1H-pyrazole-4-carbonitrile, 1-(4-amino-3-methylbenzyl)-3-(pentafluoroethyl)-N-[3-(trifluoromethyl)phenyl]-1H-pyrazole-4-carboxamide, 1-(4-amino-3-methylbenzyl)-3-(pentafluoroethyl)-N-[4-(trifluoromethyl)phenyl]-1H-pyrazole-4-carboxamide, 1-(4-amino-3-methylbenzyl)-3-(pentafluoroethyl)-N-phenyl-1H-pyrazole-4-carboxamide, 1-(4-amino-3-methylbenzyl)-4-[3-(trifluoromethyl)phenyl]-1,4-dihydro-5H-tetrazol-5-one, 1-(4-amino-3-methylbenzyl)-4-[3,5-bis(trifluoromethyl)phenyl]-1,4-dihydro-5H-tetrazol-5-one, 1-(4-amino-3-methylbenzyl)-4-[4-(trifluoromethyl)phenyl]-1,4-dihydro-5H-tetrazol-5-one, 1-(4-amino-3-methylbenzyl)-5-methyl-3-(pentafluoroethyl)-1,2,4-triazin-6(1H)-one, 1-(4-amino-3-methylbenzyl)-N-[2,2,2-trifluoroethyl)-3-(trifluoromethyl)-1H-pyrazole-4-carboxamide, 1-(4-amino-3-methylbenzyl)-N-(2-cyano-3-methylbutan-2-yl)-3-(trifluoromethyl)-1H-pyrazole-4-carboxamide, 1-(4-amino-3-methylbenzyl)-N-(4-chlorophenyl)-3-(pentafluoroethyl)-1H-pyrazole-4-carboxamide, 1-(4-amino-3-methylbenzyl)-N-(4-methylphenyl)-3-(pentafluoroethyl)-1H-pyrazole-4-carboxamide, 1-(4-amino-3-methylbenzyl)-N-(4-methoxyphenyl)-3-(pentafluoroethyl)-1H-pyrazole-4-carboxamide, 1-(4-amino-3-methylbenzyl)-N-(propane-2-yl)-3-(trifluoromethyl)-1H-pyrazole-4-carboxamide, 1-(4-amino-3-methylbenzyl)-N,N-dimethyl-3-(trifluoromethyl)-1H-pyrazole-4-carboxamide, 1-(4-amino-3-methylbenzyl)-N,N′-bis(2,2,2-trifluoroethyl)-1H-pyrazole-3,5-dicarboxamide, 1-(4-amino-3-methylbenzyl)-N-[3,4-bis(trifluoromethyl)phenyl]-3-(trifluoromethyl)-1H-pyrazole-4-carboxamide, 1-(4-amino-3-methylbenzyl)-N-[3,4-bis(trifluoro-methyl)phenyl]-3-(pentafluoroethyl)-1H-pyrazole-4-carboxamide, 1-(4-amino-3-methylbenzyl)-N-3,5-bis(trifluoro-methyl)phenyl]-3-(trifluoromethyl)-1H-pyrazole-4-carboxamide, 1-(4-amino-3-methylbenzyl)-N-[3,5-bis(trifluoromethyl)phenyl]-3-(pentafluoroethyl)-1H-pyrazole-4-carboxamide, 1-(4-amino-3-methylbenzyl)-N-[3,5-bis(trifluoromethyl)phenyl]-N-methyl-3-(trifluoromethyl)-1H-pyrazole-4-carboxamide, 1-[1-(4-amino-3-methylbenzyl)-3-(trifluoromethyl)-1H-pyrazol-4-yl]-2,2,2-trifluoroethanol, 1-[1-(4-amino-3-methylbenzyl)-3-(trifluoromethyl)-1H-pyrazol-4-yl]-2,2,2-trifluoroethanone-O-methyloxime, 1-[1-(4-amino-3-methylbenzyl)-3-(pentafluoroethyl)-1H-pyrazol-4-yl]-2,2,3,3,3-pentafluoropropan-1-one, 1-[1-(4-amino-3-methylbenzyl)-3-(pentafluoroethyl)-1H-pyrazol-5-yl]-2,2,2-trifluoroethanone, 1-[1-(4-amino-3-methylbenzyl)-4-(pentafluoroethyl)-1H-pyrrol-2-yl]-2,2,2-trifluoroethanol, 1-[2-(4-amino-3-methylphenoxy)ethyl]-4-[3,5-bis((trifluoromethyl)phenyl)-1,4-dihydro-5H-tetrazol-5-one, 1-[3,5-bis(trifluoromethyl)phenyl]-4-[4-(ethylamino)-3-methylbenzyl]-1,4-dihydro-5H-tetrazol-5-one, 1-[4-(ethylamino)-3-methylbenzyl]-4-[3-(trifluoromethyl)phenyl]-1,4-dihydro-5H-tetrazol-5-one, 1-[4-(ethylamino)-3-methylbenzyl]-4-[4-(trifluoromethyl)phenyl]-1,4-dihydro-5H-tetrazol-5-one, 2-(4-amino-3-methylbenzyl]-4-(2-chlorophenyl)-5-(trifluoromethyl)-2,4-dihydro-3H-1,2,4-triazol-3-one, 2-(4-amino-3-methylbenzyl]-4-(difluoromethyl)-5-(pentafluoroethyl)-2,4-dihydro-3H-1,2,4-triazol-3-one, 2-(4-amino-3-methylbenzyl]-4-[3,5-bis(trifluoromethyl)phenyl]-5-(trifluoromethyl)-2,4-dihydro-3H-1,2,4-triazol-3-one, 2-[(5-amino-6-methylpyridine-2-yl)methyl]-4-cyclopropyl-5-(trifluoromethyl)-2,4-dihydro-3H-1,2,4-triazol-3-one, 2-[(5-amino-6-methylpyridine-2-yl)methyl]-4-methyl-5-(trifluoromethyl)-2,4-dihydro-3H-1,2,4-triazol-3-one, 2-{[3,5-bis(trifluoromethyl)-1H-1,2,4-triazol-1-yl]methyl}-4-methylaniline, 2-{[3,5-bis(trifluoromethyl)-1H-1,2,4-triazol-1-yl]methyl}-4-chloroaniline, 2-{[3,5-bis(trifluoromethyl)-1H-1,2,4-triazol-1-yl]methyl}-5-fluoro-4-methylaniline, 2-{[3,5-bis(trifluoromethyl)-1H-pyrazol-1-yl]methyl}-5-fluoro-6-methylpyridin-3-amine, 2-amino-5-({-4-[3,5-bis(trifluoromethyl)phenyl]-5-oxo-4,5-dihydro-1H-tetrazol-1-yl}methyl)-N-(propan-2-yl)benzamide, 2-amino-5-{[3,5-bis(trifluoromethyl)-1H-pyrazol-1-yl]methyl}benzonitrile, 2-chloro-4-{[3-(pentafluoroethyl)-5-(trifluoromethyl)-1H-1,2,4-triazol-1-yl]methyl}aniline, 2-methyl-3-({3-(pentafluoroethyl)-4-[4-(trifluoromethyl)phenyl]-1H-pyrazol-1-yl}methyl)aniline, 2-methyl-3-({3-(pentafluoroethyl)-5-[4-(trifluoromethyl)phenyl]-1H-1,2,4-triazole-1-yl}methyl)aniline, 2-methyl-3-({5-[4-(trifluoromethyl)phenyl]-2H-tetrazol-2-yl}methyl)aniline, 2-methyl-3-{[5-{4-[(methylthio)methyl]phenyl}-3-(pentafluoroethyl)-1H-1,2,4-triazol-1-yl]methyl}aniline, 2-methyl-4-({3-(trifluoromethyl)-4-[4-(trifluoromethyl)phenyl]-1H-pyrazol-1-yl}methyl)aniline, 2-methyl-4-({3-(trifluoromethyl)-5-[(trifluoromethyl)thio]-1H-pyrazol-1-yl}methyl)aniline, 2-methyl-4-({3-(trifluoromethyl)-5-[3-(trifluoromethyl)phenyl]-1H-pyrazol-1-yl}methyl)aniline, 2-methyl-4-({3-(trifluoromethyl)-5-[4-(trifluoromethyl)pyridin-3-yl]-1H-1,2,4-triazol-1-yl}methyl)aniline, 2-methyl-4-({3-(trifluoromethyl)-5-[5-(trifluoromethyl)pyridin-2-yl]-1H-1,2,4-triazol-1-yl}methyl)aniline, 2-methyl-4-({3-(pentafluoroethyl)-4-[(trimethylsilyl)ethynyl]-1H-pyrazol-1-yl}methyl)aniline, 2-methyl-4-({3-(pentafluoroethyl)-5-[(2,2,2-trifluoroethyl)thio]-1H-1,2,4-triazole-1-yl}methyl)aniline, 2-methyl-4-({3-(pentafluoroethyl)-5-[(2,2,2-trifluoroethyl)thio]-1H-pyrazol-1-yl}methyl)aniline, 2-methyl-4-({3-(pentafluoroethyl)-5-[(trifluoromethyl)thio]-1H-pyrazol-1-yl}methyl)aniline, 2-methyl-4-({3-(pentafluoroethyl)-5-[5-(trifluoromethyl)pyridin-2-yl]-1H-1,2,4-triazole-1-yl}methyl)aniline, 2-methyl-4-({3-[3-(trifluoromethyl)phenyl]-1H-pyrazol-1-yl}methyl)aniline, 2-methyl-4-({3-[4-(trifluoromethyl)phenyl]-1H-pyrazol-1-yl}methyl)aniline, 2-methyl-4-({3-phenyl-4-[3-(trifluoromethyl)-1H-1,2,4-triazol-1-yl]-1H-pyrazol-1-yl}methyl)aniline, 2-methyl-4-({4-(pentafluoroethyl)-3-[3-(trifluoromethyl)phenyl]-1H-pyrazol-1-yl}methyl)aniline, 2-methyl-4-({4-(pentafluoroethyl)-3-[4-(trifluoromethyl)phenyl]-1H-pyrazole-1-yl}methyl)aniline, 2-methyl-4-({5-(trifluoromethyl)-3-[3-(trifluoromethyl)phenyl]-1H-pyrazol-1-yl}methyl)aniline, 2-methyl-4-({543-(trifluoromethyl)phenyl]-2H-tetrazol-2-yl}methyl)aniline, 2-methyl-4-({5-[4-(trifluoromethyl)phenyl]-2H-tetrazol-2-yl}methyl)aniline, 2-methyl-4-[3-(pentafluoroethyl)-5-(trifluoromethyl)-1H-1,2,4-triazol-1-yl]methyl}aniline, 2-methyl-4-{[1-methyl-5-(trifluoromethyl)-1H-1,2,4-triazol-3-yl]methyl}aniline, 2-methyl-4-{[3-(1,1,2,2-tetrafluoroethyl)-5-(trifluoromethyl)-1H-1,2,4-triazol-1-yl]methyl}aniline, 2-methyl-4-{[3-(trifluoromethyl)-1H-1,2,4-triazol-1-yl]methyl}aniline, 2-methyl-4-[3-(trifluoromethyl)-1H-pyrazol-1-yl]methyl}aniline, 2-methyl-4-{[3-(pentafluoroethyl)-1H-1,2,4-triazol-1-yl]methyl}aniline, 2-methyl-4-{[3-(pentafluoroethyl)-1H-1,2,4-triazol-5-yl]methyl}aniline, 2-methyl-4-{[3-(pentafluoroethyl)-1H-pyrazol-1-yl]methyl}aniline, 2-methyl-4-{[3-(pentafluoroethyl)-1H-pyrazol-5-yl]methyl}aniline, 2-methyl-4-{[3-(pentafluoroethyl)-4-(trifluoromethyl)-1H-pyrazol-1-yl]methyl}aniline, 2-methyl-4-{[3-(pentafluoroethyl)-5-(thiophen-2-yl)-1H-1,2,4-triazol-1-yl]methyl}aniline, 2-methyl-4-{[3-(pentafluoroethyl)-5-(trifluoromethyl)-1H-1,2,4-triazol-1-yl]methyl}aniline, 2-methyl-4-{[3-(pentafluoroethyl)-5-(trifluoromethyl)-1H-pyrazol-1-yl]methyl}aniline, 2-methyl-4-{[3-(pentafluoroethyl)-5-(trifluoromethyl)-4,5-dihydro-1H-1,2,4-triazol-1-yl]methyl}aniline, 2-methyl-4-{[3-phenyl-5-(trifluoromethyl)-1H-1,2,4-triazol-1-yl]methyl}aniline, 2-methyl-4-{[4-(2,2,2-trifluoroethoxy)-6-(trifluoromethyl)pyrimidin-2-yl]methyl}aniline, 2-methyl-4-{[4-(pentafluoroethyl)-6-(trifluoromethyl)pyrimidin-2-yl]methyl}aniline, 2-methyl-4-{[5-(1,1,2,2-tetrafluoroethyl)-3-(trifluoromethyl)-1H-1,2,4-triazol-1-yl]methyl}aniline, 2-methyl-4-{[5-(2,2,2-trifluoroethyl)-3-(trifluoromethyl)-1H-1,2,4-triazol-1-yl]methyl}aniline, 2-methyl-4-{[5-(propan-2-ylthio)-3-(trifluoromethyl)-1H-1,2,4-triazol-1-yl]methyl}aniline, -methyl-4-{[5-(pentadecafluoroheptyl)-1,2,4-oxaziazol-3-yl]methyl}aniline, 2-methyl-4-{[5-(pentafluoroethyl)-1,2,4-oxaziazol-3-yl]methyl}aniline, 2-methyl-4-{[5-(pentafluoroethyl)-3-(trifluoromethyl)-1H-1,2,4-triazol-1-yl]methyl}aniline, 2-methyl-4-{[5-(pentylthio)-3-(trifluoromethyl)-1H-1,2,4-triazol-1-yl]methyl}aniline, 2-methyl-4-{[5-(methylthio)-3-(pentafluoroethyl)-1H-1,2,4-triazol-1-yl]methyl}aniline, 2-methyl-4-{[5-methyl-3-(pentafluoroethyl)-1H-1,2,4-triazol-1-yl]methyl}aniline, 2-methyl-4-{1-[3-(heptafluoropropyl)-1H-pyrazol-1-yl]ethyl}aniline, 2-methyl-4-{1-[3-(pentafluoroethyl)-1H-pyrazol-1-yl]ethyl}aniline, 2-methyl-6-[1-methyl-5-(trifluoromethyl)-1H-pyrazol-3-yl]oxy}pyridine-3-amine, 2-methyl-6-{[3-(1,1,2,2-tetrafluoroethyl)-1H-1,2,4-triazol-1-yl]methyl}pyridin-3-amine, 2-methyl-6-{[3-(1,1,2,2-tetrafluoroethyl)-5-(trifluoromethyl)-1H-1,2,4-triazol-1-yl]methyl}pyridin-3-amine, 2-methyl-6-{[3-(pentafluoroethyl)-5-(trifluoromethyl)-1H-1,2,4-triazol-1-yl]methyl}pyridin-3-amine, 2-methyl-6-{[3-(pentafluoroethyl)-5-(trifluoromethyl)-1H-pyrazol-1-yl]methyl}pyridin-3-amine, 2-methyl-6-{[5-(trifluoromethyl)-2H-tetrazol-2-yl]methyl}pyridin-3-amine, 2-methyl-6-{[5-(pentafluoroethyl)-3-(trifluoromethyl)-1H-pyrazol-1-yl]methyl}pyridin-3-amine, 3-({5-[3-chloro-5-(trifluoromethyl)pyridin-2-yl]-3-(pentafluoroethyl)-1H-1,2,4-triazol-1-yl]methyl)-2-methylaniline, 3-(trifluoromethyl)-4-[3-(trifluoromethyl)-1H-pyrazol-1-yl]aniline, 3-{[3,4-bis(pentafluoroethyl)-1H-pyrazol-1-yl]methyl}-2-methyl aniline, 3-{[3,5-bis(trifluoromethyl)-1H-1,2,4-triazol-1-yl]methyl}-2-methylaniline, 3-{[3,5-bis(trifluoromethyl)-1H-pyrazol-1-yl]methyl}-2-methylaniline, 3-{[3,5-bis(pentafluoroethyl)-1H-pyrazol-1-yl]methyl}-2-methylaniline, 3-{[4-iodo-3-[(pentafluoroethyl)-1H-pyrazol-1-yl]methyl}-2-methylaniline, 3-{[5-(4-chlorophenyl)-3-(pentafluoroethyl)-1H-1,2,4-triazol-1-yl]methyl}-2-methylaniline, 3-{[5-(5-chlorothiophen-2-yl)-3-(pentafluoroethyl)-1H-1,2,4-triazol-1-yl]methyl}-2-methylaniline, 3-{[5-(6-chloropyridin-3-yl)-3-(pentafluoroethyl)-1H-1,2,4-triazol-1-yl]methyl}-2-methylaniline, 3-{[5-{[3,5-bis(trifluoromethyl)-1H-pyrazol-1-yl]methyl}-3-(pentafluoroethyl)-1H-1,2,4-triazol-1-yl]methyl}-2-methylaniline, 3-fluoro-4-{[3-(1,1,2,2-tetrafluoroethyl)-5-(trifluoromethyl)-1H-1,2,4-triazol-1-yl]methyl}aniline, 3-fluoro-4-{[5-(1,1,2,2-tetrafluoroethyl)-3-(trifluoromethyl)-1H-1,2,4-triazol-1-yl]methyl}aniline, 4-({1-[3-chloro-5-(trifluoromethyl)pyridin-2-yl]-3-(trifluoromethyl)-1H-1,2,4-triazol-5-yl}methyl)-2-methylaniline, 4-({3-(4-chlorophenyl)-4-[3-(trifluoromethyl)-1H-1,2,4-triazol-1-yl]-1H-pyrazol-1-yl}methyl)-2-methylaniline, 4-({3,5-bis[chloro(difluoro)methyl]-1H-1,2,4-triazol-1-yl}methyl)-2-methylaniline, 4-({3-[3,5-bis(trifluoromethyl)phenyl]-1H-pyrazol-1-yl}methyl)-2-methylaniline, 4-({3-[3,5-bis(trifluoromethyl)phenyl]-4-[3-(trifluoromethyl)-1H-1,2,4-triazol-1-yl]-1H-pyrazol-1-yl}methyl)-2-methylaniline, 4-({3-[3,5-bis(trifluoromethyl)phenyl]-5-(trifluoromethyl)-1H-1,2,4-triazol-1-yl}methyl)-2-methylaniline, 4-({3-[chloro(difluoro)methyl]-5-(trifluoromethyl)-1H-1,2,4-triazol-1-yl]methyl)-2-methylaniline, 4-({3-[chloro(difluoro)methyl]-5-(pentafluoroethyl)-1H-1,2,4-triazol-1-yl}methyl)-2-methylaniline, 4-({3-t-butyl-4-[3-(trifluoromethyl)-1H-1,2,4-triazol-1-yl]-1H-pyrazol-1-yl}methyl)-2-methylaniline, 4-({4-[3,5-bis(trifluoromethyl)phenyl]-1H-pyrazol-1-yl}methyl)-2-methylaniline, 4-({-4-[3,5-bis(trifluoromethyl)phenyl]-3-(pentafluoroethyl)-1H-pyrazol-1-yl}methyl)-2-methylaniline, 4-({4-[3,5-bis(trifluoromethyl)phenyl]-3,5-bis(trifluoromethyl)-1H-pyrazol-1-yl}methyl)-2-methylaniline, 4-({-4-([5-(3,5-dichlorophenyl)-5-(trifluoromethyl)-4,5-dihydroisoxazol-3-yl]-3-(trifluoromethyl)-1H-pyrazol-1-yl}methyl)-2-methylaniline, 4-({4-chloro-3′-4-(difluoromethoxy)phenyl]-1′H-1,4′-bipyrazol-1′-yl}methyl)-2-methylaniline, 4-({4-iodo-3-[3-(trifluoromethyl)phenyl]-1H-pyrazol-1-yl}methyl)-2-methylaniline, 4-({4-iodo-3-[4-(trifluoromethyl)phenyl]-1H-pyrazol-1-yl}methyl)-2-methylaniline, 4-({5-[2,6-bis(pentafluoroethyl)pyridin-4-yl]-3-(trifluoromethyl)-1H-1,2,4-triazol-1-yl}methyl)-2-methylaniline, 4-({5-[3,5-bis(trifluoromethyl)phenyl]-1,2,4-oxaziazol-3-yl}methyl)-2-methylaniline, 4-({5-[3,5-bis(trifluoromethyl)phenyl]-1,3,4-oxaziazole-2-yl}methyl)-2-methylaniline, 4-({5-[3,5-bis(trifluoromethyl)phenyl]-2H-tetrazol-2-yl}methyl)-2-methylaniline, 4-({5-[3-chloro-5-(trifluoromethyl)pyridin-2-yl]-3-(trifluoromethyl)-1H-1,2,4-triazol-1-yl}methyl)-2-methylaniline, 4-({5-[chloro(difluoro)methyl]-3-(trifluoromethyl)-1H-1,2,4-triazol-1-yl}methyl)-2-methylaniline, 4-({5-chloro(difluoro)methyl]-3-(pentafluoroethyl)-1H-1,2,4-triazol-1-yl}methyl)-2-methylaniline, 4-(4-amino-3-methylbenzyl)-2-[3,5-bis(trifluoromethyl)phenyl]-5-(trifluoromethyl)-2,4-dihydro-3H-1,2,4-triazol-3-one, 4-[(3-[3,5-bis(trifluoromethyl)-1H-pyrazol-1-yl]methyl}-1H-pyrazol-1-yl)methyl]-2-methylaniline, 4-[1,1,1,3,3,3-hexafluoro-2-(1H-1,2,4-triazole-1-yl)propan-2-yl]-2-methylaniline, 4-[2,2,2-trifluoro-1-(1H-1,2,4-triazol-1-yl)ethyl]aniline, 4-[3,5-bis(trifluoromethyl)-1H-1,2,4-triazol-1-yl]-2-methylaniline, 4-[3,5-bis(trifluoromethyl)-1H-pyrazol-1-yl]-2-methylaniline, 4-[3,5-bis(trifluoromethyl)-4H-1,2,4-triazol-4-yl]-2-methylaniline, 4-[3,5-bis(pentafluoroethyl)-4H-1,2,4-triazol-4-yl]-2-methylaniline, 4-{[1-(2,2,2-trifluoroethyl)-3-(trifluoromethyl)-1H-1,2,4-triazol-5-yl]methyl}aniline, 4-{[1-(difluoromethyl)-3-(pentafluoroethyl)-1H-1,2,4-triazol-5-yl]methyl}-2-methylaniline, 4-{[1-(difluoromethyl)-5-(pentafluoroethyl)-1H-1,2,4-triazol-3-yl]methyl}-2-methylaniline, 4-{[1-ethyl-5-(pentadecafluoroheptyl)-1H-1,2,4-triazol-3-yl]methyl}-2-methylaniline, 4-{[3-(pentafluoroethyl)-5-(trifluoromethyl)-1H-1,2,4-triazol-1-yl]methyl}aniline, 4-{[3,4-diiodo-5-(pentafluoroethyl)-1H-pyrazol-1-yl]methyl}-2-methylaniline, 4-{[3,4-bis(pentafluoroethyl)-1H-pyrazol-1-yl]methyl}-2,6-diiodoaniline, 4-{[3,4-bis(pentafluoroethyl)-1H-pyrazol-1-yl]methyl}-2-fluoro-6-iodoaniline, 4-{[3,4-bis(pentafluoroethyl)-1H-pyrazol-1-yl]methyl}-2-fluoroaniline, 4-{[3,4-bis(pentafluoroethyl)-1H-pyrazol-1-yl]methyl}-2-methylaniline, 4-{[3,4-bis(pentafluoroethyl)-1H-pyrazol-1-yl]methyl}-2-iodoaniline, 4-{[3,4-bis(pentafluoroethyl)-1H-pyrazol-1-yl]methyl}-N,2-dimethylaniline, 4-{[3,4-bis(pentafluoroethyl)-1H-pyrazol-1-yl]methyl}aniline, 4-{[3,5-bis(1,1,2,2-tetrafluoroethyl)-1H-1,2,4-triazol-1-yl]methyl}-2-methylaniline, 4-{[3,5-bis(trifluoromethyl)-1H-1,2,4-triazol-1-yl]methyl}-2,6-dichloroaniline, 4-{[3,5-bis(trifluoromethyl)-1H-1,2,4-triazol-1-yl]methyl}-2,6-dibromoaniline, 4-{[3,5-bis(trifluoromethyl)-1H-1,2,4-triazol-1-yl]methyl}-2-chloro-3-fluoroaniline, 4-{[3,5-bis(trifluoromethyl)-1H-1,2,4-triazol-1-yl]methyl}-2-chloro-5-fluoroaniline, 4-{[3,5-bis(trifluoromethyl)-1H-1,2,4-triazol-1-yl]methyl}-2-chloro-6-methylaniline, 4-{[3,5-bis(trifluoromethyl)-1H-1,2,4-triazol-1-yl]methyl}-2-chloroaniline, 4-{[3,5-bis(trifluoromethyl)-1H-1,2,4-triazol-1-yl]methyl}-2-fluoroaniline, 4-{[3,5-bis(trifluoromethyl)-1H-1,2,4-triazol-1-yl]methyl}-2-bromoaniline, 4-{[3,5-bis(trifluoromethyl)-1H-1,2,4-triazol-1-yl]methyl}-2-methylaniline, 4-{[3,5-bis(trifluoromethyl)-1H-1,2,4-triazol-1-yl]methyl}-3-fluoro-2-methylaniline, 4-{[3,5-bis(trifluoromethyl)-1H-1,2,4-triazol-1-yl]methyl}-3-fluoroaniline, 4-{[3,5-bis(trifluoromethyl)-1H-1,2,4-triazol-1-yl]methyl}-5-fluoro-2-methylaniline, 4-{[3,5-bis(trifluoromethyl)-1H-1,2,4-triazol-1-yl]methyl}aniline, 4-{[3,5-bis(trifluoromethyl)-1H-pyrazol-1-yl]methyl}-2-(methylthio)aniline, 4-{[3,5-bis(trifluoromethyl)-1H-pyrazol-1-yl]methyl}-2,6-dichloroaniline, 4-{[3,5-bis(trifluoromethyl)-1H-pyrazol-1-yl]methyl}-2,6-bromoaniline, 4-{[3,5-bis(trifluoromethyl)-1H-pyrazol-1-yl]methyl}-2-[(methylthio)methyl]aniline, 4-{[3,5-bis(trifluoromethyl)-1H-pyrazol-1-yl]methyl}-2-chloro-6-methylaniline, E4-{[3,5-bis(trifluoromethyl)-1H-pyrazol-1-yl]methyl}-2-chloroaniline, E4-{[3,5-bis(trifluoromethyl)-1H-pyrazol-1-yl]methyl}-2-fluoroaniline, 4-{[3,5-bis(trifluoromethyl)-1H-pyrazol-1-yl]methyl}-2-bromoaniline, 4-{[3,5-bis(trifluoromethyl)-1H-pyrazol-1-yl]methyl}-2-methylaniline, 4-{[3,5-bis(trifluoromethyl)-1H-pyrazol-1-yl]methyl}-2-methoxyaniline, 4-{[3,5-bis(trifluoromethyl)-1H-pyrazol-1-yl]methyl}-5-fluoro-2-methylaniline, 4-{[3,5-bis(trifluoromethyl)-1H-pyrazol-1-yl]methyl}-N²,N²-dimethylbenzene-1,2-diamine, 4-{[3,5-bis(trifluoromethyl)-1H-pyrazol-1-yl]methyl}aniline, 4-{[3,5-bis(pentafluoroethyl)-1H-1,2,4-triazol-1-yl]methyl}-2-methylaniline, 4-{[3,5-bis(pentafluoroethyl)-1H-1,2,4-triazol-1-yl]methyl}aniline, 4-{[3,5-bis(pentafluoroethyl)-1H-pyrazol-1-yl]methyl}-2,6-dimethylaniline, 4-{[3-fluoro-5-(pentafluoroethyl)-4-(trifluoromethyl)-1H-pyrazol-1-yl]methyl}-2-methylaniline, 4-{[4-(heptafluoropropyl)-6-(trifluoromethyl)pyrimidin-2-yl]methyl}-2-methylaniline, 4-{[4,5-d]iodo-3-(pentafluoroethyl)-1H-pyrazol-1-yl]methyl}-2-methylaniline, 4-([4,6-bis(trifluoromethyl)pyrimidin-2-yl]methyl}-2-methylaniline, 4-{[4,6-bis(pentafluoroethyl)pyrimidin-2-yl]methyl}-2-methylaniline, 4-{[4-ethyl-3-(pentafluoroethyl)-1H-pyrazol-1-yl]methyl}-2-methylaniline, 4-{[4-bromo-3-(trifluoromethyl)-1H-pyrazol-1-yl]methyl}-2-methylaniline, 4-{[4-iodo-3-(trifluoromethyl)-1H-pyrazol-1-yl]methyl}-2-methylaniline, 4-{[4-iodo-3-(pentafluoroethyl)-1H-pyrazol-1-yl]methyl}-2-methylaniline, 4-{[5-(2-chloro-1,1,2,2-tetrafluoroethyl)-3-(pentafluoroethyl)-1H-1,2,4-triazol-1-yl]methyl}-2-methylaniline, 4-{[5-(3,5-dichloropyridin-2-yl)-3-(pentafluoroethyl)-1H-1,2,4-triazol-1-yl]methyl}-2-methylaniline, 4-{[5-(3-chloropyridin-2-yl)-3-(pentafluoroethyl)-1H-1,2,4-triazol-1-yl]methyl}-2-methylaniline, 4-{[5-(4-chlorophenyl)-3-(pentafluoroethyl)-1H-1,2,4-triazol-1-yl]methyl}-2-methylaniline, 4-{[5-(5-chloropyridin-2-yl)-3-(pentafluoroethyl)-1H-1,2,4-triazol-1-yl]methyl}-2-methylaniline, 4-{[5-(ethylthio)-3-(trifluoromethyl)-1H-1,2,4-triazol-1-yl]methyl}-2-methylaniline, 4-{[5-(dichloromethyl)-3-(pentafluoroethyl)-1H-1,2,4-triazol-1-yl]methyl}-2-methylaniline, 4-{[5-(difluoromethyl)-3-(trifluoromethyl)-1H-1,2,4-triazol-1-yl]methyl)-2-methylaniline, 4-{[5-(difluoromethoxy)-3-(trifluoromethyl)-1H-pyrazol-1-yl]methyl}-2-methylaniline, 4-{[5-(difluoromethoxy)-3-(pentafluoroethyl)-1H-pyrazol-1-yl]methyl}-2-methylaniline, 4-{[5-(furan-2-yl)-3-(pentafluoroethyl)-1H-1,2,4-triazol-1-yl]methyl}-2-methylaniline, 4-{[5-(heptafluoropropyl)-1,2,4-oxadiazol-3-yl]methyl}-2-methylaniline, 4-{[5-(heptafluoropropyl)-3-(pentafluoroethyl)-1H-1,2,4-triazol-1-yl]methyl}-2-methylaniline, 4-{[5,5-dimethyl-3-(pentafluoroethyl)-4,5-dihydro-1H-1,2,4-triazol-1-yl]methyl}-2-methylaniline, 4-{[5-t-butyl-3-(pentafluoroethyl)-1H-1,2,4-triazol-1-yl]methyl}-2-methylaniline, 4-{[5-fluoro-3-(pentafluoroethyl)-4-(trifluoromethyl)-1H-pyrazol-1-yl]methyl}-2-methylaniline, 4-{[5-bromo-3-(pentafluoroethyl)-1H-1,2,4-triazol-1-yl]ethyl}-2-methylaniline, 4-{1-[3,5-bis(trifluoromethyl)-1H-1,2,4-triazol-1-yl]ethyl}-2-methylaniline, 4-{1-[3,5-bis(trifluoromethyl)-1H-pyrazol-1-yl]ethyl)-2-methylaniline, 4-{1-[3,5-bis(trifluoromethyl)-1H-pyrazol-1-yl]propyl}-2-methylaniline, 4-{2-[3,5-bis(trifluoromethyl)-1H-pyrazol-1-yl]ethyl}-2-methylaniline, 4-{2-[3,5-bis(trifluoromethyl)-1H-pyrazol-1-yl]ethoxy}-2-methylaniline, 4-{4-[3,5-bis(trifluoromethyl)phenyl]-1H-pyrazol-1-yl}-2-methylaniline, 4-{5-[3,5-bis(trifluoromethyl)phenyl]-1,3,4-oxaziazol-2-yl]-2-methylaniline, 4-amino-2-(4-amino-3-methylbenzyl)-5-(pentafluoroethyl)-2,4-dihydro-3H-1,2,4-triazol-3-one, 4-methyl-3-{[3-(trifluoromethyl)-1H-pyrazol-1-yl]methyl}aniline, 6-{[3-(heptafluoropropyl)-1H-1,2,4-triazol-1-yl]methyl}-2-methylpyridine-3-amine, 6-{[3-(heptafluoropropyl)-5-(trifluoromethyl)-1H-pyrazol-1-yl]methyl}-2-methylpyridin-3-amine, 6-{[3,4-bis(pentafluoroethyl)-1H-pyrazol-1-yl]methyl}-2-methylpyridin-3-amine, 6-{[3,5-bis(1,1,2,2-tetrafluoroethyl)-1H-1,2,4-triazol-1-yl]methyl}-2-methylpyridin-3-amine, 6-{[3,5-bis(trifluoromethyl)-1H-1,2,4-triazol-1-yl]methyl}-2-methylpyridin-3-amine, 6-{[3,5-bis(trifluoromethyl)-1H-1,2,4-triazol-1-yl]methyl}-4-methylpyridin-3-amine, 6-{[3,5-bis(trifluoromethyl)-1H-pyrazol-1-yl]methyl}-2-chloropyridin-3-amine, 6-{[3,5-bis(trifluoromethyl)-1H-pyrazol-1-yl]methyl}-2-methylpyridin-3-amine, 6-{[3,5-bis(trifluoromethyl)-1H-pyrazol-1-yl]methyl}-4-methylpyridin-3-amine, 6-{[3,5-bis(trifluoromethyl)-1H-pyrazol-1-yl]methyl}-5-fluoro-2-methylpyridin-3-amine, 6-{[3,5-bis(pentafluoroethyl)-1H-1,2,4-triazol-1-yl]methyl}-2-methylpyridin-3-amine, 6-{[3,5-bis(pentafluoroethyl)-1H-pyrazol-1-yl]methyl}-2-methylpyridin-3-amine, 6-{[4,5-dichloro-2-(trifluoromethyl)-1H-imidazol-1-yl]methyl}-2-methylpyridin-3-amine, 6-{[4-chloro-3-(heptafluoropropyl)-5-methyl-1H-pyrazol-1-yl]methyl}-2-methylpyridin-3-amine, 6-{[4-chloro-3,5-bis(difluoromethyl)-1H-pyrazol-1-yl]methyl}-2-methylpyridin-3-amine, 6-{[4-bromo-3,5-bis(difluoromethyl)-1H-pyrazol-1-yl]methyl}-2-methylpyridin-3-amine, 6-{[5-(heptafluoropropyl)-2H-tetrazol-2-yl]methyl}-2-methylpyridine-3-amine, 6-methyl-N²-[1-methyl-3-(pentafluoroethyl)-4-(trifluoromethyl)-1H-pyrazol-5-yl]pyridine-2,5-diamine, N-[1-(4-amino-3-methylbenzyl)-3-(trifluoromethyl)-1H-1,2,4-triazol-5-yl]-2,2,2-trifluoroacetamide, N-[4-(4-aminobenzyl)-1H-imidazol-2-yl]-3-fluoro-4-(trifluoromethyl)benzamide, N-[5-(4-aminobenzyl)-1H-imidazol-2-yl]-3-fluoro-4-(trifluoromethyl)benzamide, N²,6-dimethyl-N²-[1-methyl-3-(pentafluoroethyl)-4-(trifluoromethyl)-1H-pyrazol-5-yl]pyridine-2,5-diamine, N²-{1-[2,6-dichloro-4-(trifluoromethyl)phenyl]-3-(trifluoromethyl)-4-[(trifluoromethyl)thio]-1H-pyrazol-5-yl}-6-methylpyridine-2,5-diamine, N-ethyl-2-methyl-4-{[3-(pentafluoroethyl)-5-(trifluoromethyl)-1H-1,2,4-triazol-1-yl]methyl}aniline, ethyl (4-amino-3-methylphenyl)[4,6-bis(trifluoromethyl)pyrimidin-2-yl]acetate, ethyl[1-(4-amino-3-methylbenzyl)-3-(trifluoromethyl)-1H-pyrazol-4-yl](difluoro)acetate, ethyl 1-(4-amino-3-methylbenzyl)-3-(trifluoromethyl)-1H-pyrazole-4-carboxylate, methyl (4-amino-3-methylphenyl)[4,6-bis(trifluoromethyl)pyrimidin-2-yl]acetate, methyl 2-amino-4-{[3,5-bis(trifluoromethyl)-1H-1,2,4-triazol-1-yl]methyl}benzoate, and methyl 2-amino-5-({4-[3,5-bis(trifluoromethyl)phenyl]-5-oxo-4,5-dihydro-1H-tetrazol-1-yl)methyl)benzoate.

Some of the compounds of the formula (XIV) are known compounds described in Japanese Patent Application Laid-Open No. 2004-277333, Japanese Patent Application Laid-Open No. 2006-76990 or WO 2006/053643.

The compounds of the formula (XIV) are synthesized according to the process described below.

When A represents, among the herein definitions, CH(CN), CH(CH₃), CH(CO₂CH₃), CH(CO₂C₂H₅) or alkylene which may be interrupted by O, S or C(═O) and Q represents, among the herein definitions, a heterocyclic group having a free atomic valence on a nitrogen atom in the structure, compounds of the formulas (XIVa), (XIVb) and (XIVc) may be obtained by reacting the compounds represented by the formulas (XIXa), (XIXb) and (XIXc) below with the compound represented by the formula (XX) below:

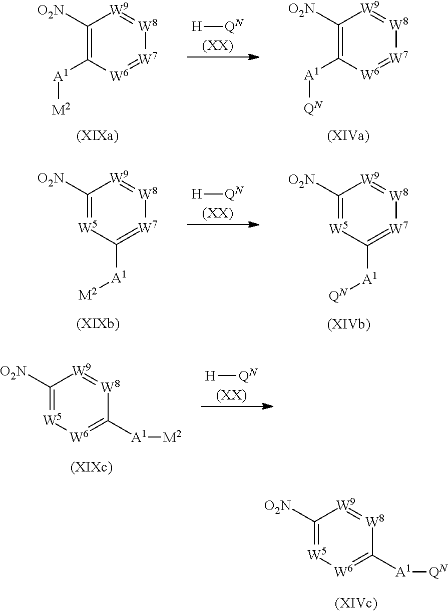

wherein A¹ represents CH(CN), CH(CH₃), CH(CO₂CH₃), CH(CO₂C₂H₅) or alkylene which may be interrupted by O, S or C(═O), M² represents chloro, bromo or methylsulfonyloxy, Q^{N} represents a heterocyclic group having a hydrogen atom on a nitrogen atom in the structure and W⁵, W⁶, W⁷, W⁸ and W⁹ are as defined herein.

The compounds of the formulas (XIXa), (XIXb) and (XIXc) are commercially available and well known in the field of organic chemistry or may be readily synthesized according to a process well known in the field of organic chemistry, such as the process described in J. Chem. Soc., (1976), p. 416 and a process in which a hydroxyalkyl group is halogenated with thionyl chloride and the process described in J. Org. Chem., vol. 58 (1993), pp. 272-274 in which the esterification to methane sulfonate is carried out with methanesulfonyl chloride. Alternatively, they may be readily synthesized according to a process well known in the field of organic chemistry and described in Organic Synthesis Collective, vol. 2 (1943), p. 443 and vol. 4 (1963), p. 921, in which the corresponding alkyl group is halogenated with bromine, N-chlorosuccinimide (NCS) or N-bromosuccinimide (NBS).

When A¹ is COCH₂, they may be readily synthesized according to a process well known in the field of organic chemistry and described in, for example, Organic Synthesis Collective, vol. 2 (1943), p. 480, in which the corresponding COCH₃ group is halogenated with bromine.

Specific examples of the compounds of the formula (XIXa) include the following compounds:

2-nitrobenzyl chloride, 2-nitrobenzyl bromide, 2-nitro-5-chlorobenzyl chloride, 5-methyl-2-nitrobenzyl chloride, 4-fluoro-5-methyl-2-nitrobenzyl bromide, and 2-nitrophenacyl bromide.

Specific examples of the compounds of the formula (XIXb) include the following compounds: 3-nitrobenzyl chloride, 3-nitrobenzyl bromide, 2-methyl-3-nitrobenzyl chloride, 4-methyl-3-nitrobenzyl chloride, and 3-nitrophenacyl bromide.

Specific examples of the compounds of the formula (XIXc) include the following compounds: 4-nitrobenzyl chloride, 4-nitrobenzyl bromide, 2-methyl-4-nitrobenzyl chloride, 3-methyl-4-nitrobenzyl chloride, methanesulfonic acid 4-nitrobenzyl ester, methanesulfonic acid 2-methyl-4-nitrobenzyl ester, methanesulfonic acid 3-methyl-4-nitrobenzyl ester, 4-nitro-3-methylbenzenesulfonyl chloride, 3-fluoro-4-nitrobenzyl bromide, 3-chloro-4-nitrobenzyl chloride, 2-fluoro-5-methyl-4-nitrobenzyl chloride, 4-nitrophenethyl bromide, 1-(3-chloropropyl)-4-nitrobenzene, 2-chloroethyl 4-nitrophenyl ether, 2-chloroethyl 3-methyl-4-nitrophenyl ether, 6-(bromomethyl)-2-methyl-3-nitropyridine, methanesulfonic acid (4-methyl-5-nitropyridin-2-yl)methyl, 4-nitrophenacyl bromide, and 3-methyl-4-nitrophenacyl bromide.

The compounds of the formula (XX) include known compounds, and specific examples thereof include the following compounds: 1-[3-(trifluoromethyl)phenyl]-1,4-dihydro-5H-tetrazol-5-one, 1-[3,5-bis(trifluoromethyl)phenyl]-1,4-dihydro-5H-tetrazol-5-one, 1-[4-(trifluoromethyl)phenyl]-1,4-dihydro-5H-tetrazole-5-one, 2-(trifluoroacetyl)-1H-pyrrole, 2,4-bis(trifluoromethyl)-1H-imidazole, 3-(trifluoromethyl)-1H-1,2,4-triazole, 3-(trifluoromethyl)-1H-pyrazole, 3-(hexafluoro-n-propyl)-1H-pyrazole, 3-(pentafluoroethyl)-1H-1,2,4-triazole, 3-(pentafluoroethyl)-1H-pyrazole, 3-(pentafluoroethyl)-5-(trifluoromethyl)-1H-1,2,4-triazole, 3,4-bis(pentafluoroethyl)-1H-pyrazole, 3,5-bis(trifluoromethyl)-1H-1,2,4-triazole, 3,5-bis(trifluoromethyl)-1H-pyrazole, 3,5-bis(pentafluoroethyl)-1H-1,2,4-triazole, 3,5-bis(pentafluoroethyl)-1H-pyrazole, 3-[3-(trifluoromethyl)phenyl]-1,1-pyrazole, 3-[4-(trifluoromethyl)phenyl]-1H-pyrazole, 4-(trifluoromethyl)-2H-1,2,3-trizole, 4-(pentafluoroethyl)-1H-pyrazole, 4-chloro-3-(trifluoromethyl)-1H-pyrazole, 4-bromo-3-(trifluoromethyl)-1H-pyrazole, 4-iodo-2-(trifluoroacetyl)-1H-pyrrole, 4-iodo-3-(pentafluoroethyl)-1H-pyrazole, 4-iodo-3-(trifluoromethyl)-1H-pyrazole, 4-(trifluoroacetyl)-3-(trifluoromethyl)-1H-pyrazole, 3-(trifluoromethyl)-1H-pyrazole-4-carboxylic acid ethyl ester, 3-(pentafluoroethyl)-1H-pyrazole-4-carboxylic acid ethyl ester, 5-(trifluoromethyl)-1H-tetrazole, and 5-[4-(trifluoromethyl)phenyl]-1H-tetrazole.

The reaction of the compound of the formula (XIXa), (XIXb) or (XIXc) with the compound of the formula (XX) may be carried out in an appropriate diluent, and examples of diluents used therefor include aliphatic, alicyclic and aromatic hydrocarbons (optionally chlorinated), such as pentane, hexane, cyclohexane, petroleum ether, ligroin, benzene, toluene, xylene and dichloromethane; ethers, such as ethyl ether, methyl ethyl ether, isopropyl ether, butyl ether, dioxane, dimethoxyethane (DME), tetrahydrofuran (THF) and diethylene glycol dimethyl ether (DGM); ketones, such as acetone, methyl ethyl ketone (MEK), methyl-isopropyl ketone and methyl isobutyl ketone (MIBK); nitriles, such as acetonitrile, propionitrile and acrylonitrile; esters, such as ethyl acetate and amyl acetate; and acid amides, such as dimethyl formamide (DMF), dimethylacetamide (DMA), N-methylpyrrolidone, 1,3-dimethyl-2-imidazolidinone and hexamethylphosphoric triamide (HMPA).

The reaction may be carried out in the presence of an acid-binding agent, and examples thereof include, for example, inorganic bases, including hydrides, hydroxides, carbonates and bicarbonates of alkali metals or alkaline earth metals, such as sodium hydride, lithium hydride, sodium hydrogen carbonate, potassium hydrogen carbonate, sodium carbonate, potassium carbonate, lithium hydroxide, sodium hydroxide, potassium hydroxide and calcium hydroxide; and inorganic alkali metal amides, such as lithium amide, sodium amide or potassium amide; and organic bases, including alcolates, tertiary amines, dialkylaminoanilines and pyridines, such as triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO) or 1,8-diazabicyclo[5,4,0]undec-7-ene (DBU).

Furthermore, the reaction may be carried out also by using a phase transfer catalyst in the presence of a diluent. Examples of the diluents used therefor include water; aliphatic, alicyclic and aromatic hydrocarbons (optionally chlorinated), such as pentane, hexane, cyclohexane, petroleum ether, ligroin, benzene, toluene and xylene; and ethers, such as ethyl ether, methyl ethyl ether, isopropyl ether, butyl ether, dioxane, dimethoxyethane (DME), tetrahydrofuran (THF) and diethylene glycol dimethyl ether (DGM). Example of phase transfer catalysts include quaternary ions, such as tetramethylammonium bromide, tetrapropylammonium bromide, tetrabutylammonium bromide, tetrabutylammonium bissulfate, tetrabutylammonium iodide, trioctylmethylammonium chloride, benzyltriethylammonium bromide, butylpyridinium bromide, heptylpyridinium bromide and benzyltriethylammonium chloride; crown ethers, such as dibenzo-18-crown-6, dicyclohexyl-18-crown-6 or 18-crown-6; cryptand, such as [2.2.2]-cryptate, [2.1.1]-cryptate, [2.2.1]-cryptate, [2.2.B]-cryptate and [3.2.2]-cryptate.

The reaction may be conducted in a substantially wide range of temperature. Generally, the reaction may be carried out at a temperature of from about 0 to about 200° C., preferably from room temperature to about 150° C. This reaction is desirably carried out under normal pressure although it may also be operated under increased or reduced pressure.

When the reaction is carried out, for example, 1 mole of the compound of the formula (XIXa), (XIXb) or (Mc) may be reacted with 1 mole or slightly excess mole amount of the compound of the formula (XX) in the presence of potassium carbonate in a diluent, such as acetonitrile, to obtain the objective compound of the formula (XIVa), (XIVb) or (XIVc).

Specific examples of the compounds of the formula (XIVa) include the following compounds: 1-(4-chloro-2-nitrobenzyl)-3,5-bis(trifluoromethyl)-1H-1,2,4-triazole, 1-(5-methyl-2-nitrobenzyl)-3,5-bis(trifluoromethyl)-1H-1,2,4-triazole, 1-(4-fluoro-5-methyl-2-nitrobenzyl)-3,5-bis(trifluoromethyl)-1H-1,2,4-triazole, and 2-{[3,5-bis(trifluoromethyl)-1H-pyrazol-1-yl]methyl}-5-fluoro-6-methyl-3-nitropyridine.

Specific examples of the compounds of the formula (XIVb) include the following compounds: 1-(2-methyl-3-nitrobenzyl)-3,5-bis(trifluoromethyl)-1H-pyrazole, 1-(2-methyl-3-nitrobenzyl)-3,5-bis(trifluoromethyl)-1H-1,2,4-triazole, 1-(2-methyl-3-nitrobenzyl)-3,4-bis(pentafluoroethyl)-1H-pyrazole, 1-(2-methyl-3-nitrobenzyl)-4-iodo-4-(pentafluoroethyl)-1H-pyrazole, 1-(2-methyl-3-nitrobenzyl)-3,5-bis(pentafluoroethyl)-1H-pyrazole, and

1-(2-methyl-3-nitrobenzyl)-5-[4-(trifluoromethyl)phenyl]-2H-tetrazole.

Specific examples of the compounds of the formula (XIVc) include the following compounds: 1-(4-nitrobenzyl)-3,5-bis(trifluoromethyl)-1H-pyrazole, 1-(3-methyl-4-nitrobenzyl)-3,5-bis(trifluoromethyl)-1H-pyrazole, 1-(3-methyl-4-nitrobenzyl)-5-pentafluoroethyl-3-trifluoromethyl-1H-[1,2,4]-triazole, 1-(3-methyl-4-nitrobenzyl)-3-pentafluoroethyl-5-trifluoromethyl-1H-[1,2,4]-triazole, 1-(3-methyl-4-nitrobenzyl)-3,4-bis(pentafluoroethyl)-1H-pyrazole, 6-{[3,5-bis(trifluoromethyl)-1H-pyrazol-1-yl]methyl}-2-methyl-3-nitropyridine, 2-{[3,5-bis(trifluoromethyl)-1H-pyrazol-1-yl]methyl}-3-fluoro-6-methyl-5-nitropyridine, 6-{[3,5-bis(pentafluoroethyl)-1H-pyrazol-1-yl]methyl}-2-methyl-3-nitropyridine, 6-{[3,5-bis(trifluoromethyl)-1H-1,2,4-triazol-1-yl]methyl}-2-methyl-3-nitropyridine, and 2-methyl-3-nitro-6-{[5-(trifluoromethyl)-2H-tetrazol-2-yl]methyl}pyridine.

When, in the formula (XIV), W⁷ represents C-(A)r-Q, r is 0 and Q represents, among the above definitions, a heterocyclic group having a free atomic valence on a nitrogen atom in the structure, the compound of the formula (XIVd) is obtained by reacting a compound represented by the following formula (XXI) with the compound of the formula (XX):

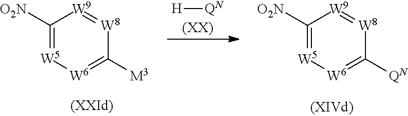

wherein M³ represents fluoro or chloro, and Q^{N}, W⁵, W⁶, W⁸ and W⁹ are as defined herein.

The compounds of the formula (XXI) are well known in the field of organic chemistry, and examples thereof include commercially available compounds: 1-fluoro-4-nitrobenzene, 1-chloro-4-nitrobenzene, 2-fluoro-5-nitrotoluene, 5-fluoro-2-nitrotoluene, 2-chloro-5-nitropyridine and 2-chloro-4-methyl-5-nitropyridine.

The compounds of the formula (XX) are as enumerated herein.

The reaction of the compound of the formula (XXI) with the compound of the formula (XX) may be carried out in an appropriate diluent, and examples of the diluents used therefor include aromatic hydrocarbons (optionally chlorinated), such as chlorobenzene, dichlorobenzene, toluene and xylene; ethers, such as butyl ether, dioxane, dimethoxyethane (DME), tetrahydrofuran (THF) and diethylene glycol dimethyl ether (DGM); nitriles, such as acetonitrile and propionitrile; and acid amides, such as dimethyl formamide (DMF), dimethyl acetamide (DMA), N-methylpyrrolidone, 1,3-dimethyl-2-imidazolidinone and hexamethylphosphoric triamide (HMPA).

The reaction may be carried out in the presence of an acid binding agent, and examples thereof include inorganic bases, including hydrides, hydroxides, carbonates and bicarbonates of alkali metals or alkaline earth metals, such as sodium hydride, lithium hydride, sodium hydrogen carbonate, potassium hydrogen carbonate, sodium carbonate, potassium carbonate, lithium hydroxide, sodium hydroxide, potassium hydroxide and calcium hydroxide; and inorganic alkali metal amides, such as lithium amide, sodium amide and potassium amide.

The reaction may be conducted in a substantially wide range of temperature. Generally, the reaction may be carried out at a temperature of from about 0 to about 200° C., preferably from room temperature to about 150° C. This reaction is desirably carried out under normal pressure although it may also be operated under increased or reduced pressure.

When the reaction is carried out, for example, 1 mole of the compound of the formula (XXI) may be reacted with 1 mole or slightly excess mole amount of the compound of the formula (XX) in the presence of potassium carbonate in a diluent, such as DMF, to obtain the objective compound of the formula (XIVd).

Specific examples of compounds of the formula (XIVd) include the following compounds: 1-(4-nitrophenyl)-3-(trifluoromethyl)-1H-pyrazole, 1-(3-methyl-4-nitrophenyl)-3-(trifluoromethyl)-1H-pyrazole, 1-(3-methyl-4-nitrophenyl)-3-(pentafluoroethyl)-1H-pyrazole, 1-(3-methyl-4-nitrophenyl)-3,5-bis(trifluoromethyl)-1H-pyrazole and 1-(3-methyl-4-nitrophenyl)-3,4-bis(pentafluoroethyl)-1H-pyrazole.

When, in the formula (XIV), W⁷ represents C-(A)r-Q, r is 0, Q is Q38 and R⁴ and R⁵ represent C₁₋₄ fluoroalkyl, the compound of the formula (XIVe) may be obtained by reacting the compound represented by the following formula (XXII) with the compound of the formula (XXIII):

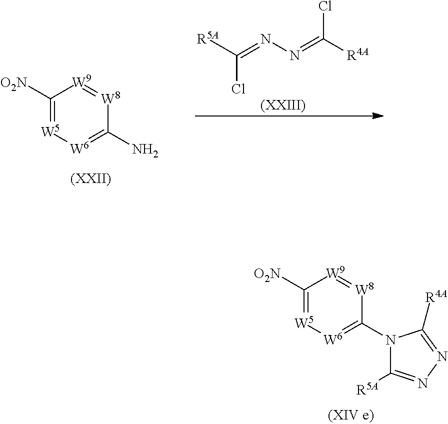

wherein R^{4A} and R^{5A} represent C₁₋₄ fluoroalkyl, and W⁵, W⁶, W⁸ and W⁹ are as defined herein.

The compounds of the formula (XXII) are well known in the field of organic chemistry and include commercially available compounds: 4-nitroaniline, 2-methyl-4-nitroaniline, 3-methyl-4-nitroaniline and 2-chloro-4-nitroaniline.

The compounds of the formula (XXIII) are known compounds described in J Fluorine Chem., vol. 65 (1993), pp. 101-110 and examples thereof include the following compounds: 1,4-dichloro-1,4-bis(trifluoromethyl)-1,3-diazatetra-1,3-diene, 1,4-dichloro-1,4-bis(pentafluoroethyl)-1,3-diazatetra-1,3-diene and 1,4-dichloro-1,4-bis(heptafluoropropyl)-1,3-diazatetra-1,3-diene.

The reaction of the compound of the formula (XXII) with the compound of the formula (XXIII) may be carried out in an appropriate diluent, and examples of diluents used therefor include aliphatic, alicyclic and aromatic hydrocarbons (optionally chlorinated), such as pentane, hexane, cyclohexane, petroleum ether, ligroin, benzene, toluene, xylene and dichloromethane; ethers, such as ethyl ether, methyl ethyl ether, isopropyl ether, butyl ether, dioxane, dimethoxyethane (DME), tetrahydrofuran (THF) and diethylene glycol dimethyl ether (DGM); ketones, such as acetone, methyl ethyl ketone (MEK), methyl-isopropyl ketone and methyl isobutyl ketone (MIBK); nitriles, such as acetonitrile, propionitrile and acrylonitrile; and esters, such as ethyl acetate and amyl acetate.

The reaction may be carried out in the presence of an acid-binding agent, and examples thereof include inorganic bases, including hydrides, hydroxides, carbonates and bicarbonates of alkali metals or alkaline earth metals, such as sodium hydride, lithium hydride, sodium hydrogen carbonate, potassium hydrogen carbonate, sodium carbonate, potassium carbonate, lithium hydroxide, sodium hydroxide, potassium hydroxide and calcium hydroxide; and inorganic alkali metal amides, such as lithium amide, sodium amide and potassium amide; and organic bases, including alcolates, tertiary amines, dialkylaminoanilines and pyridines, such as triethylamine, 1,1,4,4-tetramethylethylenediamine (TMEDA), N,N-dimethyl-aniline, N,N-diethylaniline, pyridine, 4-dimethylamino-pyridine (DMAP), 1,4-diazabicyclo[2,2,2]octane (DABCO) and 1,8-diazabicyclo[5,4,0]undec-7-ene (DBU).

The reaction may be conducted in a substantially wide range of temperature. Generally, the reaction may be carried out at a temperature of from about −60 to about 150° C., preferably from 0 to about 100° C. Further, this reaction is preferably carried out under normal pressure although it may also be operated under increased pressure or reduced pressure.

When the reaction is carried out, for example, 1 mole of the compound of the formula (XXII) may be reacted with 1 mole or slightly excess mole amount of the compound of the formula (XXIII) in the presence of triethylamine in a diluent, such as TFH, to obtain the objective compound of the formula (XIVe).

A compound wherein Q is Q35, Q36 or Q41 may be synthesized according to processes describe below.

For the reaction of the compound of the formula (XXIV) below with hydroxylamine hydrochloride followed by the derivatization to 1,2,4-oxadiazoles of the formula (XIVf), the process described in Tetrahedron Lett., vol. 42 (2001), pp. 1441-1444 can be applied. Further, the compound of the formula (XIVf) may be derivatized to the compound of the formula (XIVg) by reacting the former with hydrazine hydrate according to the process described in J. Org. Chem., vol. 68 (2003), pp. 605-608.

The compounds of formulas (XIVh) and (XIVi) may be obtained by alkylating a compound of the formula (XIVg) according to the processes well known in the field of organic chemistry:

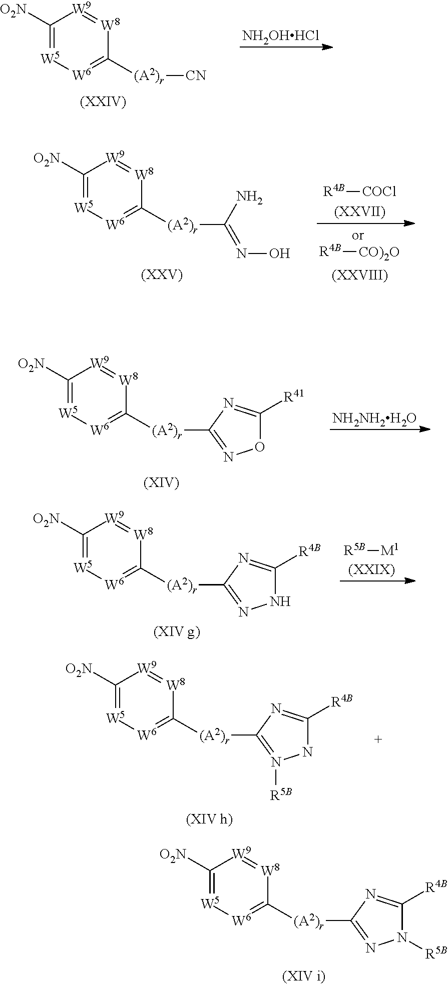

wherein A² represents alkylene, R^{5B} represents C₁₋₄ alkyl, R^{4B} represents C₁₋₄ alkyl, C₁₋₄ haloalkyl, phenyl which may be substituted or heteroaryl which may be substituted and M¹, r, W⁵, W⁶, W⁸ and W⁹ are as defined herein.

Specific examples of the compounds of the formula (XXIV) include known compounds as follows: 4-nitrobenzonitrile, 3-methyl-4-nitrobenzonitrile, (4-nitrophenyl)acetonitrile, and (3-methyl-4-nitrophenyl)acetonitrile.

some of the compounds of the formula (XXV) are known, and specific examples thereof include: 4-nitrobenzamide oxime, 3-methyl-4-nitrobenzamide oxime, 2-(4-nitrophenyl)acetamide oxime, and 2-(3-methyl-4-nitrophenyl)acetamide oxime.

The compounds of the formula (XXVII) are well known in the field of organic chemistry, and examples thereof include commercially available acetyl chloride, propionyl chloride, 2,2,3,3-tetrafluoropropionyl chloride and heptafluorobutyloyl chloride.

The compounds of the formula (XXVIII) are well known in the field of organic chemistry, and examples thereof include commercially available acetic anhydride, propionic anhydride, difluoroacetic anhydride, trifluoroacetic anhydride, chlorodifluoroacetic anhydride, pentafluoropropionic anhydride and heptafluorobutyric anhydride.

The compounds of the formula (XIVf) include known compounds, and specific examples thereof include the following compounds: 3-(4-nitrophenyl)-5-(trifluoromethyl)-1,2,4-oxadiazole, 3-(3-methyl-4-nitrophenyl)-5-(trifluoromethyl)-1,2,4-oxadiazole, 3-(4-nitrobenzyl)-5-(pentafluoroethyl)-1H-1,2,4-oxadiazole, 3-(3-methyl-4-nitrobenzyl)-5-(trifluoromethyl)-1,2,4-oxadiazole, 3-(3-methyl-4-nitrobenzyl)-5-(pentafluoroethyl)-1,2,4-oxadiazole, and 3-(3-methyl-4-nitrobenzyl)-5-(heptafluoropropyl)-1,2,4-oxadiazole.

The compounds of the formula (XIVg) include known compounds and specific examples thereof include: 3-(4-nitrophenyl)-5-(trifluoromethyl)-1H-1,2,4-triazole, 3-(3-methyl-nitrophenyl)-5-(trifluoromethyl)-1,2,4-triazole, 3-(4-nitrobenzyl)-5-(pentafluoroethyl)-1H-1,2,4-triazole, 3-(3-methyl-4-nitrobenzyl)-5-(trifluoromethyl)-1,2,4-triazole, 3-(3-methyl-4-nitrobenzyl)-5-(pentafluoroethyl)-1,2,4-triazole, and 3-(3-methyl-4-nitrobenzyl)-5-(heptafluoropropyl)-1,2,4-triazole.

The compounds of the formula (XXIX) are compounds well known in the field of organic chemistry, and examples thereof include commercially available methyl bromide, methyl iodide, ethyl bromide, ethyl iodide, chlorodifluoromethane, bromodifluoromethane, 3-iodo-1,1,1-trifluoropropane and 3-bromo-1,1,1-trifluoropropane.

The compounds of the formula (XIVh) are novel, and specific examples thereof include the following compounds: 1-methyl-5-(4-nitrophenyl)-3-(trifluoromethyl)-1H-1,2,4-triazole, 1-(difluoromethyl)-5-(3-methyl-4-nitrophenyl)-3-(trifluoromethyl)-1H-1,2,4-triazole, 1-(difluoromethyl)-5-(4-nitrobenzyl)-3-(pentafluoroethyl)-1H-1,2,4-triazole, 1-methyl-5-(3-methyl-4-nitrobenzyl)-3-(trifluoromethyl)-1H-1,2,4-triazole, 1-ethyl-5-(3-methyl-4-nitrobenzyl)-3-(trifluoromethyl)-1H-1,2,4-triazole, 5-(3-methyl-4-nitrobenzyl)-1-(2,2,2-trifluoroethyl)-3-(trifluoromethyl)-1H-1,2,4-triazole, 1-(difluoromethyl)-5-(3-methyl-4-nitrobenzyl)-3-(trifluoromethyl)-1H-1,2,4-triazole, and 1-(difluoromethyl)-5-(3-methyl-4-nitrobenzyl)-3-(pentafluoroethyl)-1H-1,2,4-triazole.

The compounds of the formula (XIVi) are novel, and specific examples thereof include the following compounds: 1-methyl-3-(4-nitrophenyl)-5-(trifluoromethyl)-1H-1,2,4-triazole, 1-(difluoromethyl)-3-(3-methyl-4-nitrophenyl)-5-(trifluoromethyl)-1H-1,2,4-triazole, 1-(difluoromethyl)-3-(4-nitrobenzyl)-5-(pentafluoroethyl)-1H-1,2,4-triazole, 1-methyl-3-(3-methyl-4-nitrobenzyl)-5-(trifluoromethyl)-1H-1,2,4-triazole, 1-ethyl-3-(3-methyl-4-nitrobenzyl)-5-(trifluoromethyl)-1H-1,2,4-triazole, 3-(3-methyl-4-nitrobenzyl)-1-(2,2,2-trifluoroethyl)-5-(trifluoromethyl)-1H-1,2,4-triazole, 1-(difluoromethyl)-3-(3-methyl-4-nitrobenzyl)-5-(trifluoromethyl)-1H-1,2,4-triazole and 1-(difluoromethyl)-3-(3-methyl-4-nitrobenzyl)-5-(pentafluoroethyl)-1H-1,2,4-triazole.

The compounds of the formula (XIV) wherein W⁷ is C—CH₂-Q51 or C—CH(CN)-Q51 may be synthesized according to the following reaction scheme:

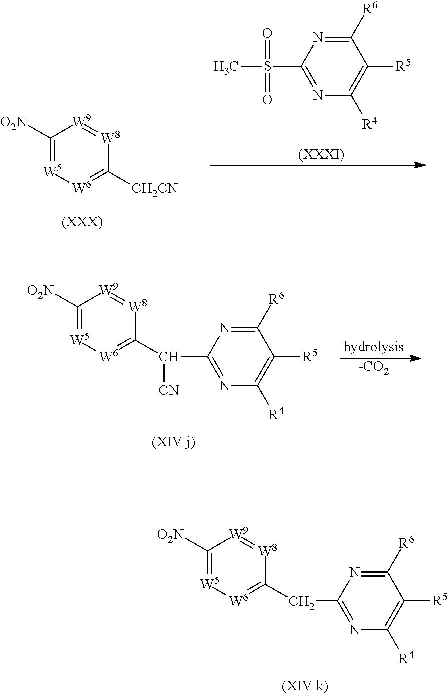

wherein W⁵, W⁶, W⁸, W⁹, R⁴, R⁵ and R⁶ are as defined herein.

The reaction of the compound of the formula (XXX) with the compound of the formula (XXXI) may be carried out according to the process described in Synth. Commun., vol. 23 (1993), pp. 591-599.

The reaction from the compound of the formula (XIVj) to a compound of the formula (XIVk) may be carried out according to the method described in J. Am. Chem. Soc., vol. 73 (1951), p. 3856.

Specific examples of the compounds of the formula (XXX) include the following known compounds: (4-nitrophenyl)acetonitrile, and (3-methyl-4-nitrophenyl)acetonitrile.

The compounds of the formula (XXXI) may be known compounds described in Japanese Patent Application Laid-open No. 2006-76990 or synthesized according to the process described in Japanese Patent Application Laid-open No. 2006-76990.

Specific examples of the compounds of the formula (XXXI) may include the following compounds: 2-methanesulfonyl-4,6-bis(trifluoromethyl)-pyridine, 2-methanesulfonyl-4-(pentafluoroethyl)-6-(trifluoromethyl)-pyridine, 2-methanesulfonyl-4-(heptafluoropropyl)-6-(trifluoromethyl)-pyrimidine and 2-methanesulfonyl-4,6-bis(pentafluoroethyl)-pyrimidine.

The compounds of the formula (XIVj) are novel, and specific examples thereof include: [4,6-bis(trifluoromethyl)pyrimidin-2-yl](3-methyl-4-nitrophenyl)acetonitrile, [4-(pentafluoroethyl)-6-(trifluoromethyl)pyrimidin-2-yl](3-methyl-4-nitrophenyl)acetonitrile, [4-(heptafluoropropyl)-6-(trifluoromethyl)pyrimidin-2-yl](3-methyl-4-nitrophenyl)acetonitrile, and [4,6-bis(pentafluoroethyl)pyrimidin-2-yl](3-methyl-4-nitrophenyl)acetonitrile.

The compounds of the formula (XIVk) are novel, and specific examples thereof may include: 2-(3-methyl-4-nitrobenzyl)-4,6-bis(trifluoromethyl)pyrimidine, 2-(3-methyl-4-nitrobenzyl)-4-(pentafluoroethyl)-6-(trifluoromethyl)pyrimidine, 2-(3-methyl-4-nitrobenzyl)-4-(heptafluoropropyl)-6-(trifluoromethyl)pyrimidine and 2-(3-methyl-4-nitrobenzyl)-4,6-bis(pentafluoroethyl)pyrimidine.

The compound of the formula (XIV) wherein W⁷ is C—CH(CO₂CH₃)-Q^{N} or C—CH(CO₂C₂H₅)-Q^{N} may be synthesized according to the following reaction scheme:

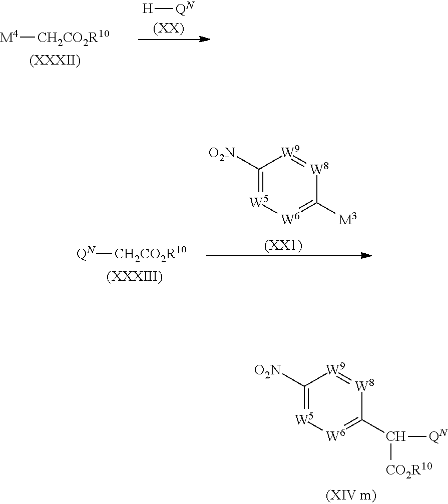

wherein M⁴ represents chloro or bromo, R¹⁰ represents methyl or ethyl, and M³, Q^{N}, W⁵, W⁶, W⁸ and W⁹ are as defined herein.

The compounds of the formula (XXXIII) may be synthesized by reacting the compounds of the formula (XX) with the compounds of the formula (XXXII).

Specific examples of the compounds of the formula (XXXII) may include commercially available compounds: chloroacetic acid methyl ester, bromoacetic acid methyl ester, chloroacetic acid ethyl ester, and bromoacetic acid ethyl ester.

The compounds of the formula (XXXIII) include known compounds described in J. Org. Chem., vol. 35 (1970), p. 3978, J. Fluorine Chem., vol. 17 (1981), pp. 179-186, J. Fluorine Chem., vol. 48 (1990), pp. 149-152 and WO 2006/53643 and the like. Specific examples the compounds may include: [2-(trifluoromethyl)-1H-imidazol-1-yl]acetic acid ethyl ester, [5-(trifluoromethyl)-2H-tetrazol-2-yl]acetic acid ethyl ester, [3,5-bis(trifluoromethyl)-1H-pyrazol-1-yl]acetic acid ethyl ester, and [3,5-bis(trifluoromethyl)-1H-1,2,4-triazol-1-yl]acetic acid ethyl ester.

Specific examples of the compounds of the formula (XXI) are as mentioned herein and may include: 1-fluoro-4-nitrobenzene, 1-chloro-4-nitrobenzene, 5-fluoro-2-nitrotoluene, 2-chloro-5-nitropyridine, 2-chloro-4-methyl-5-nitropyridine, and 2-fluoro-5-nitropyridine. The reaction of the compound of the formula (XXXIII) with the compound of the formula (XXI) may be carried out in an appropriate diluent, and examples of the diluents used therefor include aliphatic, alicyclic and aromatic hydrocarbons (optionally chlorinated), such as pentane, hexane, cyclohexane, petroleum ether, ligroin, benzene, toluene, xylene and dichloromethane; ethers, such as ethyl ether, methyl ethyl ether, isopropyl ether, butyl ether, dioxane, dimethoxyethane (DME), tetrahydrofuran (THF) and diethylene glycol dimethyl ether (DGM); ketones, such as acetone, methyl ethyl ketone (MEK), methyl-isopropyl ketone and methyl isobutyl ketone (MIBK); nitriles, such as acetonitrile, propionitrile and acrylonitrile; esters, such as ethyl acetate and amyl acetate; and acid amides, such as dimethyl formamide (DMF), dimethylacetamide (DMA), N-methylpyrrolidone, 1,3-dimethyl-2-imidazolidinone and hexamethylphosphoric triamide (HMPA).

The above reaction may be carried out in the presence of an acid-binding agent, and examples thereof include inorganic bases, including hydrides, hydroxides, carbonates and bicarbonates of alkali metals or alkaline earth metals, such as sodium hydride, lithium hydride, sodium hydrogen carbonate, potassium hydrogen carbonate, sodium carbonate, potassium carbonate, lithium hydroxide, sodium hydroxide, potassium hydroxide and calcium hydroxide; and inorganic alkali metal amides, such as lithium amide, sodium amide or potassium amide; as organic bases, including alcolates, tertiary amines, dialkylaminoanilines and pyridines, such as triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO) and 1,8-diazabicyclo[5,4,0]undec-7-ene (DBU).

The reaction may be conducted in a substantially wide range of temperature. Generally, the reaction may be carried out at a temperature of from about 0 to about 200° C., preferably from room temperature to about 150° C. Further, this reaction is preferably carried out under normal pressure although it may also be operated under increased pressure or reduced pressure.

When the reaction is carried out, for example, 1 mole of the compound of the formula (XXXIII) may be reacted with 1 mole or slightly excess mole amount of the compound of the formula (XXI) in the presence of sodium hydride in a diluent, such as DMF, to obtain the objective compound of the formula (XIVm).

The compounds of the formula (XIVm) are novel, and specific examples thereof may include the following compounds: [3,5-bis(trifluoromethyl)-1H-pyrazol-1-yl](4-nitrophenyl)acetic acid ethyl ester, [3,5-bis(trifluoromethyl)-1H-pyrazol-1-yl](3-methyl-4-nitrophenyl)acetic acid ethyl ester, [3,5-bis(trifluoromethyl)-1H-1,2,4-triazol-1-yl](4-nitrophenyl)acetic acid ethyl ester and [3,5-bis(trifluoromethyl)-1H-1,2,4-triazol-1-yl](3-methyl-4-nitrophenyl)acetic acid ethyl ester.

The compound of the formula (XIV) wherein W⁷ is C—CH₂-Q25, C—C(═O)-Q25 or C—C(═NOCH₃)— Q25 may be synthesized according to the following reaction scheme:

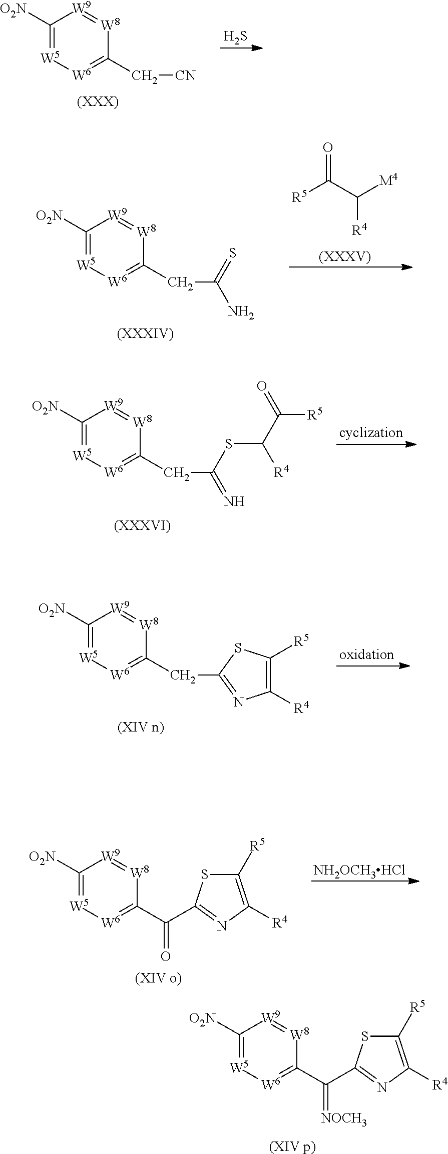

wherein M⁴, W⁵, W⁶, W⁸, W⁹, R⁴ and R⁵ are as defined herein.

The reaction of the compound of the formula (XXX) with hydrogen sulfide may be carried out according to the process described in Japanese Patent Application Laid-open No. 2006-76990.

Specific examples of compounds represented by the formula (XXX) include the aforementioned known compounds: (4-nitrophenyl)acetonitrile, and (3-methyl-4-nitrophenyl)acetonitrile.

The reaction of the compound of the formula (XXX) with hydrogen sulfide may be carried out in an appropriate diluent, and examples of diluents used therefor include pyridine, ethanol and isopropanol.

The reaction of the compound of the formula (XXX) with hydrogen sulfide may be carried out in the presence of tertiary amines, such as triethylamine.

The reaction of the compound of the formula (XXVIb) with hydrogen sulfide may be conducted in a substantially wide range of temperature. Generally, the reaction may be carried out at a temperature of from about 0 to about 200° C., preferably from room temperature to about 150° C. Further, this reaction is preferably carried out under normal pressure although it may also be operated under increased pressure or reduced pressure.

When the reaction of the compound of the formula (XXX) with hydrogen sulfide is carrying out, 1 mole of the compound of the formula (XXX) is reacted with an excess mole amount of hydrogen sulfide in the presence of triethylamine in a diluent such as pyridine to obtain a compound of the formula (XXXIV).

Compounds of the formula (XXXIV) include known compounds described in J. Org. Chem., vol. 47 (1982), pp. 4594-4595 and Japanese Patent Application Laid-open No. 2006-76990, and specific examples thereof may include: (4-nitrophenyl)thioacetoamide and (3-methyl-4-nitrophenyl)thioacetoamide.

The reaction of the compound of the formula (XXXIV) and the compound of the formula (XXXV) may be carried out according to a method described in J. Chem. Soc., 1967, p 1269 to 1273 and Japanese Patent Application Laid-open No. 2006-76990.

Specific examples of the compounds of the formula (XXXV) may include commercially available 3-bromo-1,1,1-(trifluoro)-2-propanone and 1-bromo-3,3,4,4,4-(pentafluoro)-2-butanone.

The reaction of the compound of the formula (XXXIV) with a compound of the formula (XXXV) may be carried out in an appropriate diluent, and examples of the diluents used therefor include aromatic hydrocarbons (optionally chlorinated), such as chlorobenzene, dichlorobenzene, toluene and xylene; ethers, such as butyl ether, dioxane, dimethoxyethane (DME), tetrahydrofuran (THF) and diethylene glycol dimethyl ether (DGM); nitriles, such as acetonitrile or propionitrile; and acid amides such as dimethyl formamide (DMF), dimethyl acetamide (DMA), N-methylpyrrolidone, 1,3-dimethyl-2-imidazolidinone and hexamethyl phosphoric triamide (HMPA).

The reaction of the compound of the formula (XXXIV) with a compound of the formula (XXXV) may be carried out in the presence of an acid binding agent, and examples thereof may include inorganic bases including hydrides, hydroxides, carbonates and bicarbonates of alkali metals or alkaline earth metals, such as sodium hydride, lithium hydride, sodium hydrogen carbonate, potassium hydrogen carbonate, sodium carbonate, potassium carbonate, lithium hydroxide, sodium hydroxide, potassium hydroxide and calcium hydroxide; and inorganic alkali metal amides, such as lithium amide, sodium amide or potassium amide; and organic bases, including alcolates, tertiary amines, dialkylaminoanilines and pyridines, such as triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO) and 1,8-diazabicyclo[5,4,0]undec-7-ene (DBU).

The reaction may be conducted in a substantially wide range of temperature. Generally, the reaction may be carried out at a temperature of from about 0 to about 200° C., preferably from room temperature to about 150° C. Further, this reaction is preferably carried out under normal pressure although it may also be operated under increased pressure or reduced pressure.

When the reaction of the compound of the formula (XXXIV) with a compound of the formula (XXXV) is carried out, 1 mole of the compound of the formula (XXXIV) may be reacted with 1 mole or slightly excess mole amount of the compound of the formula (XXXV) in the presence of potassium carbonate in a diluent, such as DMF, to obtain a compound of the formula (XXXVI).

The compounds of the formula (XXXVI) include known compounds described in Japanese Patent Application Laid-open No. 2006-76990, and specific examples thereof may include: 2-(4-nitrophenyl)-thioacetimidic acid 3,3,3-(trifluoro)-2-oxo-propyl ester, 2-(4-nitrophenyl)-thioacetimidic acid 3,3,4,4,4-(pentafluoro)-2-oxo-butyl ester, 2-(3-methyl-4-nitrophenyl)-thioacetimidic acid 3,3,3-(trifluoro)-2-oxo-propyl ester, and 2-(3-methyl-4-nitrophenyl)-thioacetimidic acid 3,3,4,4,4-(pentafluoro)-2-oxo-butyl ester.

The cyclic condensation reaction of the compound of the formula (XXXVI) may be carried out according to the process described in Japanese Patent Application Laid-open No. 2006-76990.

The cyclic condensation reaction of the compound of the formula (XXXVI) may be carried out in an appropriate diluent, and examples of the diluents used therefor include dichloromethane, ethanol, benzene, toluene and dioxane.

The cyclic condensation reaction of the compound of the formula (XXXVI) may be carried out in the presence of a condensation agent, and examples thereof include trifluoroacetic anhydride and pentafluoropropionic anhydride.

The cyclic condensation reaction of the compound of the formula (XXXVI) may be conducted in a substantially wide range of temperature. Generally, the reaction may be carried out at a temperature of from about 0 to about 200° C., preferably from room temperature to about 100° C. Further, this reaction is preferably carried out under normal pressure although it may also be operated under increased pressure or reduced pressure.

When the cyclic condensation reaction of the compound of the formula (XXXVI) is carried out, for example, 1 mole of the compound of the formula (XXXVI) may be reacted in the presence of 1 mole or slightly excess amount of trifluoroacetic anhydride in a diluent, such as dichloromethane, to obtain a compound of the formula (XIVn).

The compounds of the formula (XIVn) include known compounds described in Japanese Patent Application Laid-open No. 2006-76990, and specific examples thereof may include: 2-(4-nitrobenzyl)-4-(trifluoromethyl)-1,3-thiazole, 2-(4-nitrobenzyl)-4-(pentafluoroethyl)-1,3-thiazole, 2-(3-methyl-4-nitrobenzyl)-4-(trifluoromethyl)-1,3-thiazole and 2-(3-methyl-4-nitrobenzyl)-4-(pentafluoroethyl)-1,3-thiazole.

For the oxidation reaction of the compound of the above formula (XIVn), specific examples of oxidizing agents may include manganese dioxide, selenium dioxide, potassium permanganate and ammonium cerium (N) nitrate.

The oxidation reaction of the compound of the above formula (XIVn) may be carried out in an appropriate diluent, and examples of the diluents used therefor may include water, acetonitrile, acetic acid and dichloromethane.

The oxidation reaction of the compound of the formula (XIVn) may be conducted in a substantially wide range of temperature. Generally, the reaction may be carried out at a temperature of from about 0 to about 200° C., preferably from room temperature to about 100° C. Further, this reaction is preferably carried out under normal pressure although it may also be operated under increased pressure or reduced pressure.

When the oxidation reaction of the compound of the formula (XIVn) is carried out, for example, 1 mole of the compound of the formula (XIVn) may be reacted with 1 mole or slightly excess amount of ammonium cerium (IV) nitrate in a diluent, such as acetonitrile to obtain a compound of the formula (XIVo).

The compounds of the formula (XIVo) are novel, and specific examples thereof may include the following compounds: (4-nitrophenyl)[4-(trifluoro)-1,3-thiazol-2-yl]methanone, (4-nitrophenyl)[4-(pentafluoroethyl)-1,3-thiazol-2-yl]methanone, (3-methyl-4-nitrophenyl)[4-(trifluoro)-1,3-thiazol-2-yl]methanone, and (3-methyl-4-nitrophenyl)[4-(pentafluoroethyl)-1,3-thiazol-2-yl]methanone.

The reaction of the compound of the formula (XIVo) with O-methylhydroxylammonium chloride is a process well known in the field of organic chemistry and may be carried out according to the process described in Tetrahedron, vol. 48 (1992), pp. 7251-7264.

The reaction of the compound of the formula (XIVo) with O-methylhydroxylammonium chloride may be carried out in an appropriate diluent, and examples of the diluents used therefor may include pyridine, ethanol, isopropanol and water.

The reaction of the compound of the formula (XIVo) with O-methylhydroxylammonium chloride may be carried out in the presence of an appropriate base, and examples thereof may include inorganic bases, such as sodium acetate; and organic bases such as pyridine and triethylamine.

The reaction of the compound of the formula (XIVo) with O-methylhydroxylammonium chloride may be conducted in a substantially wide range of temperature. Generally, the reaction may be carried out at a temperature of from about 0 to about 200° C., preferably from room temperature to about 100° C. Further, this reaction is preferably carried out under normal pressure although it may also be operated under increased pressure or reduced pressure.

When the reaction of the compound of the formula (XIVo) with O-methylhydroxylammonium chloride is carried out, for example, 1 mole of the compound of the formula (XIVo) may be reacted with 1 mole or slightly excess mole amount of O-methylhydroxylammonium chloride in the presence of excess pyridine in an appropriate diluent such as ethanol, to obtain the compound of the formula (XIVp).

The compounds of the formula (XIVp) are novel, and specific examples thereof may include the following compounds: (4-nitrophenyl)[4-(trifluoro)-1,3-thiazol-2-yl]methanone O-methyloxime, (4-nitrophenyl)[4-(pentafluoroethyl)-1,3-thiazol-2-yl]methanone O-methyloxime, (3-methyl-4-nitrophenyl)[4-(trifluoro)-1,3-thiazol-2-yl]methanone O-methyloxime and (3-methyl-4-nitrophenyl)[4-(pentafluoroethyl)-1,3-thiazol-2-yl]methanone O-methyloxime.

The compounds of the formula (XIV) wherein W⁵, W⁶ or W⁷ is C—CH₂-Q34 may also be synthesized according to the process via the compound of the following formula (VLa), (XLb) or (XLc):

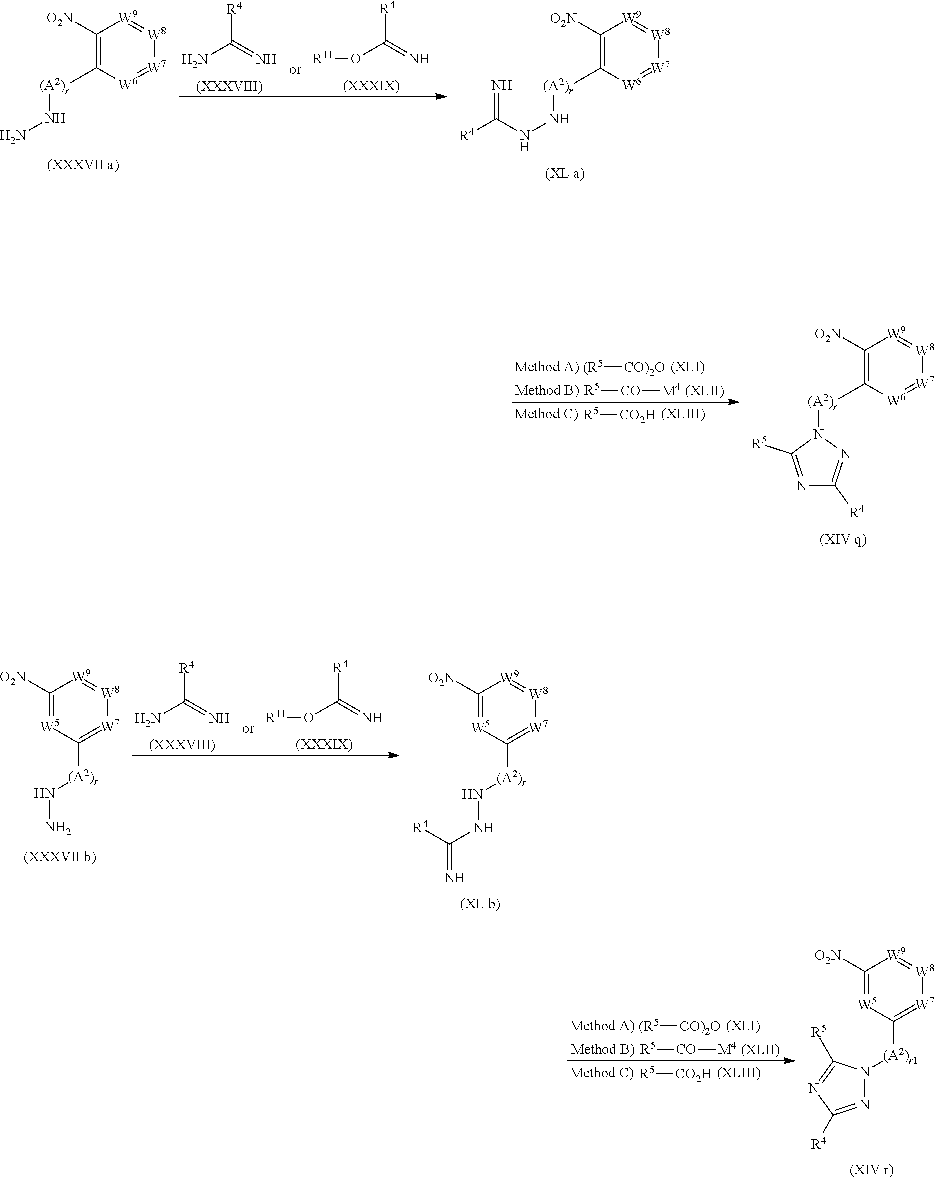

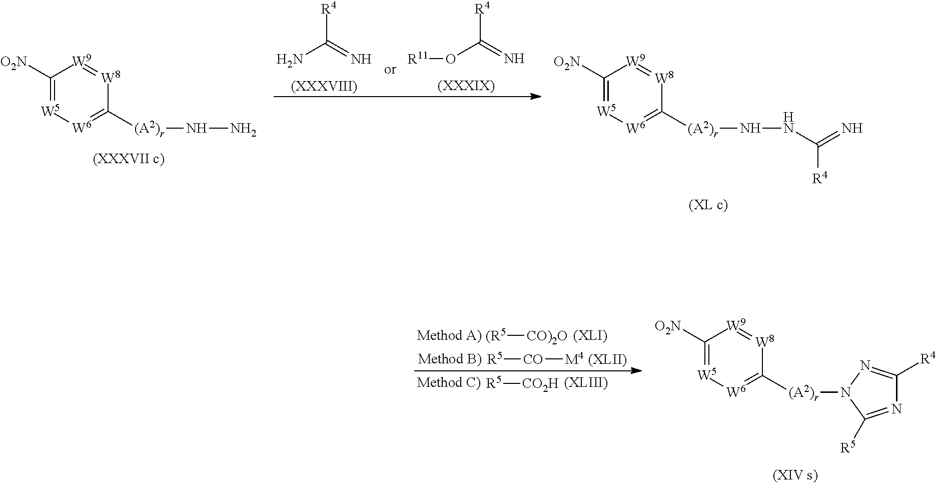

wherein R¹¹ represents C₁₋₆ alkyl or benzyl which may optionally be substituted, A² represents CH₂ or CH(CH₃), and r, M⁴, R⁴, R⁵, W⁸ and W⁹ are as define herein.

The reaction may be carried out according to the process described in J. Org. Chem., vol. 53 (1988), pp. 4349-4353.

The compounds of formulas (XXXVIIa), (XXXVIIb) and (XXXVIIc) are known or may be readily synthesized by reacting a corresponding 4-nitrofluorobenzenes or nitrobenzyl halides with hydrazine hydrate, according to the process described in Bioorg. MedChem. Lett., vol. 15 (2005), pp. 2834-2839 or Japanese Patent Application Laid-open No. 2006-76990.

Specific examples of the compounds of formulas (XXXVIIa), (XXXVIIb) and (XXXVIIc) may include the following compounds: 2-nitrophenylhydrazine, 2-nitrobenzylhydrazine, 3-nitrophenylhydrazine, 3-nitrobenzylhydrazine, (2-methyl-3-nitrobenzyl)hydrazine, (4-nitrophenyl)hydrazine, (3-methyl-4-nitrophenyl)hydrazine, (3-chloro-4-nitrophenyl)hydrazine, (4-nitrobenzyl)hydrazine, (3-methyl-4-nitrobenzyl)hydrazine, (3-chloro-4-nitrobenzyl)hydrazine, and hydrochlorides thereof.

The compounds of the formula (XXXVIII) are well known in the field of organic chemistry, and specific examples thereof may include commercially available compounds: trifluoroacetamidine, pentafluoropropylamidine, and 2,2,3,3,4,4,4-heptafluorobutylylamidine.

The compounds of the formula (XXXIX) may be known compounds described in Tetrahedron Lett., vol. 39 (1998), pp. 5565-5568 or readily synthesized by reacting corresponding haloalkyl nitriles with alcohols in accordance with the literature, and specific examples of the compounds may include the following compounds: benzimidic acid methyl ester hydrochloride, 3-(trifluoromethyl)benzimidic acid methyl ester hydrochloride, 4-(trifluoromethyl)benzimidic acid methyl ester hydrochloride, 3,5-bis(trifluoromethyl)benzimidic acid methyl ester hydrochloride, butanimidic acid ethyl ester hydrochloride, 2,2,2-trifluoroacetimidic acid methyl ester, 2,2,3,3,3-pentafluoropropionimidic acid methyl ester, 2,2,2-trifluoroacetimidic acid benzyl ester, 2,2,3,3-tetrafluoropropionimidic acid benzyl ester, trifluoroacetimidic acid 4-methoxybenzyl ester, and 2,2,3,3,3-pentafluoropropionimidic acid 4-methoxybenzyl ester.

The reaction of the compound of the formula (XXXVIIa), (XXXVIIb) or (XXXVIIc) and the compound of the formula (XXXVIII) or (XXXIX) may be carried out in an appropriate diluent, and examples of diluents used therefor include aliphatic, alicyclic and aromatic hydrocarbons (optionally chlorinated), such as pentane, hexane, cyclohexane, petroleum ether, ligroin, benzene, toluene, xylene and dichloromethane; and ethers such as ethyl ether, methyl ethyl ether, isopropyl ether, butyl ether, dioxane, dimethoxyethane (DME), tetrahydrofuran (THF) and diethylene glycol dimethyl ether (DGM).

The reaction of the compound of the formula (XXXVIIa), (XXXVIIb) or (XXXVIIc) with the compound of the formula (XXXVIII) or (XXXIX) may be conducted in a substantially wide range of temperature. Generally, the reaction may be carried out at a temperature of from about −20 to about 150° C., preferably from 0 to about 100° C. Further, this reaction is preferably carried out under normal pressure although it may also be operated under increased pressure or reduced pressure.

When the reaction of compound of the formula (XXXVIIa), (XXXVIIb) or (XXXVIIc) with a compound of the formula (XXXVIII) or (XXXIX) is carried out, for example, 1 mole of the compound of the formula (XXXVIIa), (XXXVIIb) or (XXXVIIc) may be reacted with 1 mole or slightly excess amount of the compound of the formula (XXXVIII) or (XXXIX) in an appropriate diluent, such as THF, to obtain the objective compound.

The compounds of the formulas (XLa), (XLb) and (XLc) include known compounds, and specific examples thereof may include the following compounds: N′-(4-nitrophenyl)butanimide hydrazide, N′-(4-nitrophenyl)benzenecarboxylmide hydrazide, N′-(3-methyl-4-nitrophenyl)-3-(trifluoromethyl)benzenecarboxylmide hydrazide, N′-(3-methyl-4-nitrophenyl)-4-(trifluoromethyl)benzenecarboxylmide hydrazide, N′-(3-methyl-4-nitrophenyl)-3,5-bis(trifluoromethyl)benzenecarboxylmide hydrazide, N′-(4-nitrobenzyl)benzenecarboxylmide hydrazide, N′-(3-methyl-4-nitrobenzyl)-3-(trifluoromethyl)benzenecarboxylmide hydrazide, N′-(3-methyl-4-nitrobenzyl)-4-(trifluoromethyl)benzenecarboxylmide hydrazide, N′-(3-methyl-4-nitrobenzyl)-3,5-bis(trifluoromethyl)benzenecarboxylmide hydrazide, 2,2,2-trifluoro-N′-(4-nitrophenyl)ethanimide hydrazide, 2,2,3,3,3-pentafluoro-N′-(4-nitrophenyl)propanimide hydrazide, 2,2,3,3,4,4,4-heptafluoro-N′-(4-nitrophenyl)butaneimide hydrazide, 2,2,2-trifluoro-N′-(3-methyl-4-nitrophenyl)ethanimide hydrazide, 2,2,3,3,3-pentafluoro-N′-(3-methyl-4-nitrophenyl)propanimide hydrazide, 2,2,2-trifluoro-N′-(4-nitrobenzyl)ethanimide hydrazide, 2,2,3,3,3-pentafluoro-N′-(4-nitrobenzyl)propanimide hydrazide, 2,2,3,3,4,4,4-heptafluoro-N′-(4-nitrobenzyl)butanimide hydrazide, 2,2,2-trifluoro-N′-(3-methyl-4-nitrobenzyl)ethanimide hydrazide, 2,2,3,3,3-pentafluoroN′-(3-methyl-4-nitrobenzyl)propanimide hydrazide, 2,2,3,3,4,4,4-heptafluoro-N′-(3-methyl-4-nitrobenzyl)butanimide hydrazide, 2,2,2-trifluoro-N′-(3-chloro-4-nitrobenzyl)ethanimide hydrazide, 2,2,3,3,3-pentafluoro-N′-(3-chloro-4-nitrobenzyl)propanimide hydrazide, and 2,2,3,3,4,4,4-heptafluoro-N′-(3-chloro-4-nitrobenzyl)butanimide hydrazide.

The compound of the formula (XLI), which is a starting material in the reaction with a formula (XLa), (XLb) or (XLc), is a compound well known in the field of organic chemistry. Specific examples thereof may include commercially available acetic anhydride, propionic anhydride, difluoroacetic anhydride, trifluoroacetic anhydride, chlorodifluoroacetic anhydride, pentafluoropropionic anhydride and heptafluoro-n-butyric anhydride.

The compound of the formula (XLII), which is a starting material in a reaction with a compound of the formula (XLa), (XLb) or (XLc), is a compound well known in the field of organic chemistry. Specific examples thereof may include acetyl chloride, propionyl chloride, pivaloyl chloride, difluoroacetyl chloride, trifluoroacetyl fluoride, trifluoroacetyl chloride, pentafluoropropionyl chloride, heptafluoro-n-butyloyl chloride, benzoyl chloride, 4-chlorobenzoyl chloride, thiophene-2-carbonyl chloride and furan-2-carbonyl chloride.

The compound of the formula (XLIII), which is a starting material in a reaction with a compound of the formula (XLa), (XLb) or (XLc), is a compound well known in the field of organic chemistry. Specific examples thereof may include difluoroacetic acid, trifluoroacetic acid, 3,3,3-trifluoropropionic acid, tetrafluoropropionic acid, pentafluoropropionic acid, dichloroacetic acid, chlorodifluoroacetic acid, benzoic acid, 4-chlorobenzoic acid, 3,5-dichloropyridine-2-carboxylic acid, 5-(trifluoromethyl)pyridine-2-carboxylic acid, 3-chloro-5-(trifluoromethyl)pyridine-2-carboxylic acid and 4-(trifluoromethyl)pyridine-3-carboxylic acid.

The reaction of the compound of the formula (XLa), (XLb) or (XLc) with the compound of the formula (XLI), (XLII) or (XLIII) may be carried out in an appropriate diluent, and examples of diluents used therefor may include water; aliphatic, alicyclic and aromatic hydrocarbons (optionally chlorinated) such as pentane, hexane, cyclohexane, petroleum ether, ligroin, benzene, toluene, xylene and dichloromethane; and ethers such as ethyl ether, methyl ethyl ether, isopropyl ether, butyl ether, dioxane, dimethoxyethane (DME), tetrahydrofuran (THF) and diethylene glycol dimethyl ether (DGM).

The reaction of the compound of the formula (XLa), (XLb) or (XLc) with the compound of the formula (XLI), (XLII) or (XLIII) may be conducted in a substantially wide range of temperature. Generally, the reaction may be carried out at a temperature of from about −20 to about 200° C., preferably from 0 to about 150° C. Further, this reaction is preferably carried out under normal pressure although it may also be operated under increased pressure or reduced pressure.

The reaction of the compound of the formula (XLa), (XLb) or (XLc) with the compound of the formula (XLII) is carried out in the presence of a base. Examples of the bases may include tertially amines, dialkylaminoanilines and pyridines such as triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO) and 1,8-diazabicyclo[5,4,0]undec-7-ene (DBU); and, as inorganic bases, hydroxides, carbonates and bicarbonates of alkali metal or alkaline earth metal such as sodium hydrogen carbonate, potassium hydrogen carbonate, sodium carbonate, potassium carbonate, lithium hydroxide, sodium hydroxide and potassium hydroxide.

The reaction of the compound of the formula (XLa), (XLb) or (XLc) with the compound of the formula (XLIII) is carried out in the presence of a condensation agent. Examples of the condensation agents may include carbonyldiimidazole, dicyclohexyl carbodiimide and N-(3-dimethylaminopropyl)-N′-ethylcarbodiimide hydrochloride.

When the reaction of the compound of the formula (XLa), (XLb) or (XLc) with the compound of the formula (XLI), (XLII) or (XLIII) is carried out, for example, 1 mole of the compound of the formula (XLa), (XLb) or (XLc) may be reacted with 1 mole or slightly excess amount of the compound of the formula (XLI), (XLII) or (XLIII) in the presence of 1 mole or slightly excess amount of a condensation agent, such as N-(3-dimethylaminopropyl)-N′-ethylcarbodiimide hydrochloride in a diluent, such as dioxane, to obtain the objective compound of the formula (XIVq), (XIVr) or (XIVs).

Some of the compounds of the formula (XIVq), (XIVr) or (XIVs) are described in Japanese Patent Application Laid-open No. 2006-76990.

Specific examples thereof may include the following compounds: 1-(4-nitrophenyl)-3,5-bis(trifluoromethyl)-1H-1,2,4-triazole, 1-(3-methyl-4-nitrophenyl)-3,5-bis(trifluoromethyl)-1H-1,2,4-triazole, 1-(3-methyl-4-nitrophenyl)-3-(pentafluoroethyl)-5-(trifluoromethyl)-1H-1,2,4-triazole, 1-(3-methyl-4-nitrophenyl)-5-(pentafluoroethyl)-3-(trifluoromethyl)-1H-1,2,4-triazole, 1-(4-nitrobenzyl)-3,5-bis(trifluoromethyl)-1H-1,2,4-triazole, 1-(3-methyl-4-nitrobenzyl)-3,5-bis(trifluoromethyl)-1H-1,2,4-triazole, 1-(3-methyl-4-nitrobenzyl)-3-(pentafluoroethyl)-5-(trifluoromethyl)-1H-1,2,4-triazole, 1-(3-methyl-4-nitrobenzyl)-5-(pentafluoroethyl)-3-(trifluoromethyl)-1H-1,2,4-triazole, 1-(3-methyl-4-nitrobenzyl)-3,5-bis(1,1,2,2-tetrafluoroethyl)-1H-1,2,4-triazole, 1-(3-methyl-4-nitrobenzyl)-5-(2,2,2-trifluoroethyl)-3-(trifluoromethyl)-1H-1,2,4-triazole, 3-[chloro(difluoro)methyl]-1-(3-methyl-4-nitrobenzyl)-5-(pentafluoroethyl)-1H-1,2,4-triazole, 5-(dichloromethyl)-1-(3-methyl-4-nitrobenzyl)-3-(pentafluoroethyl)-1H-1,2,4-triazole, 5-(2-chloro-1,1,2,2-tetrafluoroethyl)-1-(3-methyl-4-nitrobenzyl)-3-(pentafluoroethyl)-1H-1,2,4-triazole, 3-(heptafluoropropyl)-1-(3-methyl-4-nitrobenzyl)-5-(pentafluoroethyl)-1H-1,2,4-triazole, 5-(heptafluoropropyl)-1-(3-methyl-4-nitrobenzyl)-3-(pentafluoroethyl)-1H-1,2,4-triazole, 5-[chloro(difluoro)methyl]-1-(3-methyl-4-nitrobenzyl)-3-(trifluoromethyl)-1H-1,2,4-triazole, 5-[chloro(difluoro)methyl]-1-(3-methyl-4-nitrobenzyl)-3-(pentafluoroethyl)-1H-1,2,4-triazole, 5-methyl-1-(3-methyl-4-nitrobenzyl)-3-(pentafluoroethyl)-1H-1,2,4-triazole, 5-t-butyl-1-(3-methyl-4-nitrobenzyl)-3-(pentafluoroethyl)-1H-1,2,4-triazole, 1-(3-methyl-4-nitrobenzyl)-5-penyl-3-(trifluoromethyl)-1H-1,2,4-triazole, 5-(4-chlorophenyl)-1-(3-methyl-4-nitrobenzyl)-3-(trifluoromethyl)-1H-1,2,4-triazole, 5-chloro-2-[1-(3-methyl-4-nitrobenzyl)-3-(trifluoromethyl)-1H-1,2,4-triazol-5-yl]pyridine, 3,5-dichloro-2-[1-(3-methyl-4-nitrobenzyl)-3-(trifluoromethyl)-1H-1,2,4-triazol-5-yl]pyridine, 2-[1-(3-methyl-4-nitrobenzyl)-3-(trifluoromethyl)-1H-1,2,4-triazol-5-yl]-5-(trifluoromethyl)pyridine, 3-chloro-2-[1-(3-methyl-4-nitrobenzyl)-3-(trifluoromethyl)-1H-1,2,4-triazol-5-yl]-5-(trifluoromethyl)pyridine, 3-[1-(3-methyl-4-nitrobenzyl)-3-(trifluoromethyl)-1H-1,2,4-triazol-5-yl]-4-(trifluoromethyl)pyridine, 5-(2-furyl)-1-(3-methyl-4-nitrobenzyl)-3-(trifluoromethyl)-1H-1,2,4-triazole, 1-(3-methyl-4-nitrobenzyl)-5-(2-thienyl)-3-(trifluoromethyl)-1H-1,2,4-triazole, 1-(3-methyl-4-nitrobenzyl)-5-phenyl-3-(pentafluoroethyl)-1H-1,2,4-triazole, 5-(4-chlorophenyl)-1-(3-methyl-4-nitrobenzyl)-3-(pentafluoroethyl)-1H-1,2,4-triazole, 5-chloro-2-[1-(3-methyl-4-nitrobenzyl)-3-(pentafluoroethyl)-1H-1,2,4-triazol-5-yl]pyridine, 3,5-dichloro-2-[1-(3-methyl-4-nitrobenzyl)-3-(pentafluoroethyl)-1H-1,2,4-triazol-5-yl]pyridine, 2-[1-(3-methyl-4-nitrobenzyl)-3-(pentafluoroethyl)-1H-1,2,4-triazol-5-yl]-5-(trifluoromethyl)pyridine, 3-chloro-2-[1-(3-methyl-4-nitrobenzyl)-3-(pentafluoroethyl)-1H-1,2,4-triazol-5-yl]-5-(trifluoromethyl)pyridine, 3-[1-(3-methyl-4-nitrobenzyl)-3-(pentafluoroethyl)-1H-1,2,4-triazol-5-yl]-4-(trifluoromethyl)pyridine, 5-(2-furyl)-1-(3-methyl-4-nitrobenzyl)-3-(pentafluoroethyl)-1H-1,2,4-triazole, and 1-(3-methyl-4-nitrobenzyl)-5-(2-thienyl)-3-(pentafluoroethyl)-1H-1,2,4-triazole.

When W⁵ is CH in a formula (XIV), the compounds of the formula (XIVu) may also be obtained by reacting the compound of the formula (XIVt) with a Grignard reagent represented by the formula (XLIV) below and then carrying out the dehydrogenation reaction:

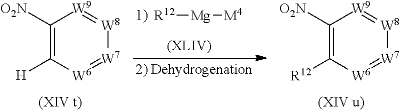

wherein R¹² represents C₁₋₄ alkyl and M⁴, W⁶, W⁷, W⁸ and W⁹ are as defined herein.
The above reaction is carried out according to the process described in Tetrahedron Letters, vol. 26 (1985), pp. 115-118.

The compounds of the formula (XIVt) can be synthesized according to the aforementioned process, and specific examples of the compounds may include: 1-(4-nitrobenzyl)-3,5-bis(trifluoromethyl)-1H-pyrazole, 1-(4-nitrobenzyl)-3,5-bis(trifluoromethyl)-1H-1,2,4-triazole, 1-(2-fluoro-4-nitrobenzyl)-3,5-bis(trifluoromethyl)-1H-pyrazole and 1-(2-fluoro-4-nitrobenzyl)-3,5-bis(trifluoromethyl)-1H-1,2,4-triazole.

The compounds of the formula (XLIV) are well known in the field of organic chemistry, and specific examples thereof may include commercially available methyl magnesium chloride, methyl magnesium bromide, ethyl magnesium chloride, ethyl magnesium bromide, isopropyl magnesium chloride and isopropyl magnesium bromide.

The reaction of the compound of the formula (XIVt) with the compound of the formula (XLIV) may be carried out in an appropriate diluent, and examples of diluents used therefor may include aliphatic, alicyclic and aromatic hydrocarbons such as pentane, hexane, cyclohexane, petroleum ether, ligroin, benzene, toluene and xylene; and ethers such as ethyl ether, methyl ethyl ether, isopropyl ether, butyl ether, dioxane, dimethoxyethane (DME), tetrahydrofuran (THF) and diethylene glycol dimethyl ether (DGM).

The dehydrogenation reaction after the reaction of the compound of the formula (XIVt) with the compound of the formula (XLIV) may be carried out in the presence of a dehydrogenating agent. Examples of the dehydrogenating agents may include 2,3-dichloro-5,6-dicyano-p-benzoquinone (DDQ).

The reaction of the compound of the formula (XIVt) with a compound of the formula (XLIV) may be conducted in a substantially wide range of temperature. Generally, the reaction may be carried out at a temperature of from about −80 to about 200° C., preferably from about −70 to about 100° C. Further, this reaction is preferably carried out under normal pressure although it may also be operated under increased pressure or reduced pressure.

When the reaction of the compound of the formula (XIVt) with the compound of the formula (XLIV) is carried out, for example, 1 mole of the compound of the formula (XIVt) may be reacted with 1 mole or slightly excess mole amount of methyl magnesium chloride in a diluent, such as THF, and then with 1 mole or slightly excess mole amount of 2,3-dichloro-5,6-dicyano-p-benzoquinone per mole of the compound of the formula (XIVt) to obtain the objective compound of the formula (XIVu).

The compounds of the formula (XIVu) include known compounds, and specific examples thereof may include the following compounds: 1-(3-methyl-4-nitrobenzyl)-3,5-bis(trifluoromethyl)-1H-pyrazole, 1-(3-methyl-4-nitrobenzyl)-3,5-bis(trifluoromethyl)-1H-1,2,4-triazole, 1-(2-fluoro-3-methyl-4-nitrobenzyl)-3,5-bis(trifluoromethyl)-1H-1,2,4-triazole and 1-(2-fluoro-3-methyl-4-nitrobenzyl)-3,5-bis(trifluoromethyl)-1H-pyrazole.

The reaction of preparation process (a) may be carried out in an appropriate diluent or a combination of appropriate diluents, and examples of the diluents used therefor may include aliphatic, alicyclic and aromatic hydrocarbons (optionally chlorinated) such as pentane, hexane, cyclohexane, petroleum ether, ligroin, benzene, toluene, xylene, dichloromethane, chloroform, carbon tetrachloride, 1,2-dichloroethane, chlorobenzene and dichlorobenzene; ethers such as ethyl ether, methyl ethyl ether, isopropyl ether, butyl ether, dioxane, dimethoxyethane (DME), tetrahydrofuran (THF) and diethylene glycol dimethyl ether (DGM); nitriles such as acetonitrile, propionitrile or acrylonitrile; and esters such as ethyl acetate and amyl acetate.

Preparation process (a) may be carried out in the presence of an acid catalyst, and examples of the acid catalysts may include mineral acids, such as hydrochloric acid and sulfuric acid; and organic acids, such as acetic acid, trifluoroacetic acid, propionic acid, methane sulfonic acid, benzene sulfonic acid and p-toluene sulfonic acid.

Preparation process (a) may be conducted in a substantially wide range of temperature. Generally, the reaction may be carried out at a temperature of from about −20 to about 100° C., preferably from about 0 to about 100° C. Further, this reaction is preferably carried out under normal pressure although it may also be operated under increased pressure or reduced pressure.

When Preparation process (a) is carried out, for example, 1 mole of the compound of the formula (II) may be reacted with 1 mole or slightly excess mole amount of the compound (III) in the presence of 0.01 to 0.1 mole of p-toluene sulfonic acid in a diluent, such as acetonitrile, to obtain the objective compound of the corresponding formula (I).

The compounds of the formula (IV), which are used as a starting material in preparation process (b), include known compounds and are synthesized according to the process described in Japanese Patent Application Laid-open No. 2006-76990. Specific examples thereof may include the following compounds: 2-(4-{[3,5-bis(trifluoromethyl)-1H-pyrazol-1-yl]methyl}-2-methylphenyl)-1H-isoindole-1,3(2H)-dione, 2-(4-{[3,5-bis(trifluoromethyl)-1H-1,2,4-triazol-1-yl]methyl}-2-methylphenyl)-1H-isoindole-1,3(2H)-dione, 2-(4-{[3,5-bis(trifluoromethyl)-1H-pyrazol-1-yl]methyl}-2-methylphenyl)-4-chloro-1H-isoindole-1,3(2H)-dione, 2-(4-{[3,5-bis(trifluoromethyl)-1H-1,2,4-triazol-1-yl]methyl}-2-methylphenyl)-4-chloro-1H-isoindole-1,3(2H)-dione, 2-(4-{[3,5-bis(trifluoromethyl)-1H-pyrazol-1-yl]methyl}-2-methylphenyl)-4-bromo-1H-isoindole-1,3(2H)-dione, 2-(4-{[3,5-bis(trifluoromethyl)-1H-1,2,4-triazol-1-yl]methyl}-2-methylphenyl)-4-bromo-1H-isoindole-1,3(2H)-dione, 2-(4-{[3,5-bis(trifluoromethyl)-1H-pyrazol-1-yl]methyl}-2-methylphenyl)-4-iodo-1H-isoindole-1,3(2H)-dione and 2-(4-{[3,5-bis(trifluoromethyl)-1H-1,2,4-triazol-1-yl]methyl}-2-methylphenyl)-4-iodo-1H-isoindole-1,3(2H)-dione.

The compounds of the formula (V), which are used as a starting material in preparation process (b), may be well known in the field of organic chemistry or synthesized according to the process described in DE-A No. 2045905 and WO 01/23350. Specific examples the compounds may include ethylamine, diethylamine, n-propylamine, isopropylamine, n-butylamine, sec-butylamine, isobutylamine, t-butylamine, t-amylamine, 2-(methylthio)-ethylamine, 2-(ethylthio)-ethylamine, 1-methyl-2-(methylthio)-ethylamine and 1,1-dimethyl-2-(methylthio)-ethylamine.

The reaction of preparation process (b) may be carried out in an appropriate diluent and examples of the diluents used therefor may include aliphatic, alicyclic and aromatic hydrocarbons (optionally chlorinated) such as pentane, hexane, cyclohexane, petroleum ether, ligroin, benzene, toluene, xylene, dichloromethane, chloroform, carbon tetrachloride, 1,2-dichloroethane, chlorobenzene and dichlorobenzene; ethers such as ethyl ether, methyl ethyl ether, isopropyl ether, butyl ether, dioxane, dimethoxyethane (DME), tetrahydrofuran (THF) and diethylene glycol dimethyl ether (DGM); esters such as ethyl acetate and amyl acetate; and acid amides such as dimethylformamide (DMF), dimethylacetamide (DMA), N-methylpyrrolidone, 1,3-dimethyl-2-imidazolidinone and hexamethylphosphoric triamide (HMPA).

Preparation process (b) may be carried out in the presence of an acid catalyst, and examples of the acid catalysts may include mineral acids such as hydrochloric acid and sulfuric acid; and organic acids such as acetic acid, trifluoroacetic acid, propionic acid, methane sulfonic acid and p-toluene sulfonic acid.

Preparation process (b) may be conducted in a substantially wide range of temperature. Generally, the reaction may be carried out at a temperature of from about −20 to about 150° C., preferably from room temperature to about 120° C. Further, this reaction is preferably carried out under normal pressure although it may also be operated under increased pressure or reduced pressure.

When preparation process (b) is carried out, for example, 1 mole of the compound of the formula (IV) may be reacted with 1 mole or slightly excess mole amount of the compound of the formula (V) in the presence of 0.01 to 0.5 mole of acetic acid in a diluent such as dioxane to obtain the objective compound of the corresponding formula (I).

The compounds of the formula (VI), which are used as a starting material in preparation process (c), include known compounds and may be synthesized according to the process described in Japanese Patent Application Laid-open No. 2006-76990. Specific examples thereof may include the following compounds: [2-(1-methylethyl)carbamoyl]benzoic acid, 3-chloro-2-(diethylcarbamoyl)benzoic acid, 3-chloro-2-{[(1S)-1-methyl-2-(methylthio)ethyl]-carbamoyl}benzoic acid, 3-bromo-2-{[(1S)-1-methyl-2-(methylthio)ethyl]-carbamoyl}benzoic acid and 3-iodo-2-{[(1S)-1-methyl-2-(methylthio)ethyl]-carbamoyl}benzoic acid.

The compounds of the formula MO, which are used as a starting material in preparation process (c), are the same as those mentioned in preparation process (a).

The reaction of preparation process (c) may be carried out in an appropriate diluent or a combination of appropriate diluents, and examples of the diluents used therefor may include aliphatic, alicyclic and aromatic hydrocarbons (optionally chlorinated) such as pentane, hexane, cyclohexane, petroleum ether, ligroin, benzene, toluene, xylene, dichloromethane, chloroform, carbon tetrachloride, 1,2-dichloroethane, chlorobenzene and dichlorobenzene; ethers such as ethyl ether, methyl ethyl ether, isopropyl ether, butyl ether, dioxane, dimethoxyethane (DME), tetrahydrofuran (THF) and diethylene glycol dimethyl ether (DGM); esters such as ethyl acetate or amyl acetate; and acid amides such as dimethylformamide (DMF), dimethylacetoamide (DMA), N-methylpyrrolidone, 1,3-dimethyl-2-imidazolinone, and hexamethylphosphoric triamide (HMPA).

The reaction of preparation process (c) is carried out either in the presence of a condensation agent such as carbonyl imidazole, dicyclohexylcarbodiimide and N-(3-dimethylaminopropyl)-N′-ethylcarbodiimide hydrochloride, or by converting the compound of the formula (VI) to the corresponding acid halide with an acid halogenating agent such as thionyl chloride and oxalyl chloride.

The reaction of preparation process (c) may be carried out in the presence of a base, and examples of bases may include tertiary amines, dialkylaminoanilines and pyridines such as triethylamine, 1,1,4,4-tetramethylethylenediamine (TMEDA), N,N-dimethylaniline, N,N-diethylaniline, pyridine, 4-dimethylamino pyridine (DMAP), 1,4-diazabicyclo[2,2,2]octane (DABCO) and 1,8-diazabicyclo[5,4,0]undec-7-ene (DBU).

The reaction of preparation process (c) may be conducted in a substantially wide range of temperature. Generally, the reaction may be carried out at a temperature of from about −20 to about 200° C., preferably from 0 to about 150° C. Further, this reaction is preferably carried out under normal pressure although it may also be operated under increased pressure or reduced pressure.

When the reaction of preparation process (c) is carried out, for example, an acid halogenating agent such as 1 mole or slightly excess amount of oxalyl chloride may be added to 1 mole of the compound of the formula (VI) in a diluent such as 1,2-dichloroethane to form an acid halide, followed by the reaction with 1 mole or slightly excess amount of (III) in the presence of a base such as 1 mole or slightly excess amount of triethylamine in a diluent such as THF to obtain the objective compound of the corresponding formula (I).

The compounds of the formula (IA), which are used as a starting material in preparation process (d), include known compounds described in Japanese Patent Application Laid-open No. 2006-76990 or may be produced according to the above preparation process (a), (b) or (c) or preparation process (f), (g), (h), (i), (j), (k) or (l) described below.

Typical examples of the compounds of the formula (IA) may include the following compounds: N¹-(4-{[3,5-bis(trifluoromethyl)-1H-pyrazol-1-yl]methyl}-2-methylphenyl)-3-iodo-N²-[(1S)-1-methyl-2-(methylthio)ethyl]-1,2-benzenedicarboxamide, N¹-(4-{[3,5-bis(trifluoromethyl)-1H-pyrazol-1-yl]methyl}-2-methylphenyl)-3-bromo-N²-[(1S)-1-methyl-2-(methylthio)ethyl]-1,2-benzenedicarboxamide, N′-(4-{[3,5-bis(trifluoromethyl)-1H-1,2,4-triazol-1-yl]methyl}-2-methylphenyl)-3-iodo-N²-[(1S)-1-methyl-2-(methylthio)ethyl]-1,2-benzenedicarboxamide, N¹-(4-{[3,5-bis(trifluoromethyl)-1H-1,2,4-triazol-1-yl]methyl}-2-methylphenyl)-3-iodo-N²-[(1S)-1-methyl-2-(methylsulfonyl)ethyl]-1,2-benzenedicarboxamide, 3-bromo-N²-[(1S)-1-methyl-2-(methylthio)ethyl]-N¹-(2-methyl-4-{[3-(pentafluoroethyl)-5-(trifluoromethyl)-1H-1,2,4-triazol-1-yl]methyl}phenyl)-1,2-benzenedicarboxamide, and 3-bromo-N²-[(1S)-1-methyl-2-(methylsulfonyl)ethyl]-N¹-(2-methyl-4-{[3-(pentafluoroethyl)-5-(trifluoromethyl)-1H-1,2,4-triazol-1-yl]methyl}phenyl)-1,2-benzenedicarboxamide.

The reaction of preparation process (d) may be carried out in an appropriate diluent or a combination of appropriate diluents, and examples of the diluents used therefor may include aliphatic, alicyclic and aromatic hydrocarbons (optionally chlorinated) such as pentane, hexane, cyclohexane, petroleum ether, ligroin, benzene, toluene, xylene, dichloromethane, chloroform, carbon tetrachloride, 1,2-dichloroethane, chlorobenzene and dichlorobenzene; ethers such as ethyl ether, methyl ethyl ether, isopropyl ether, butyl ether, dioxane, dimethoxyethane (DME), tetrahydrofuran (THF) and diethylene glycol dimethyl ether (DGM); esters such as ethyl acetate and amyl acetate; and acid amides such as dimethylformamide (DMF), dimethylacetoamide (DMA), N-methylpyrrolidone, 1,3-dimethyl-2-imidazolinone and hexamethylphosphoric triamide (HMPA).

The reaction of preparation process (d) is carried out in the presence of a cyanizing agent. Examples of the cyanizing agents include cuprous cyanide and zinc cyanide.

The reaction of preparation process (d) is carried out in the presence of a transition metal catalyst. Examples of the transition metal catalysts may include tetrakis(triphenylphosphine)palladium (0), dichlorobis(triphenylphosphine)palladium (2) and tris(dibenzylideneacetone)dipalladium chloroform complex.

The reaction of preparation process (d) may be conducted in a substantially wide range of temperature. Generally, the reaction may be carried out at a temperature of from about −20 to about 200° C., preferably from 0 to about 150° C. Further, this reaction is preferably carried out under normal pressure although it may also be operated under increased pressure or reduced pressure.

When the reaction of preparation process (d) is carried out, for example, 1 mole of the compound of the formula (IA) may be reacted with 1 mole or slightly excess amount of cuprous cyanide in the presence of a catalyst amount of tris(dibenzylideneacetone)dipalladium-chloroform complex in a diluent such as dioxane, to obtain the objective compound of the corresponding formula (I).

The compounds of the formula (IA), which are used as a starting material in preparation process (e), are as mentioned herein.

The compounds of the formula (VII), which are used as a starting material in preparation process (e), are well known in the field of organic chemistry, and specific examples thereof may include the following compounds: 2-fluorophenylboric acid, 3-fluorophenylboric acid, 4-fluorophenylboric acid, 2-(trifluoromethyl)phenylboric acid, 3-(trifluoromethyl)phenylboric acid, 4-(trifluoromethyl)phenylboric acid and 3,5-bis(trifluoromethyl)phenylboric acid.

The reaction of preparation process (e) may be carried out in an appropriate diluent or a combination of appropriate diluents, and examples of the diluents used therefor may include water; aliphatic, alicyclic and aromatic hydrocarbons (optionally chlorinated) such as pentane, hexane, cyclohexane, petroleum ether, ligroin benzene, toluene, xylene, dichloromethane, chloroform, carbon tetrachloride, 1,2-dichloroethane, chlorobenzene and dichlorobenzene; and ethers such as ethyl ether, methyl ethyl ether, isopropyl ether, butyl ether, dioxane, dimethoxyethane (DME), tetrahydrofuran (THF) and diethylene glycol dimethyl ether (DGM).

The reaction of preparation process (e) is carried out in the presence of a transition metal catalyst. Examples of the transition metal catalysts may include palladium acetate, tetrakis(triphenylphosphine)palladium (0), dichlorobis(triphenylphosphine)palladium (2) and [1,1′-(diphenylphosphino)ferrocene]dichloropalladium (2).

The reaction of preparation process (e) may be carried out in the presence of an inorganic base. Examples of the inorganic bases may include hydroxides, carbonates and bicarbonates of alkali metals or alkaline earth metals such as sodium hydrogen carbonate, potassium hydrogen carbonate, sodium carbonate, potassium carbonate, lithium hydroxide, sodium hydroxide, potassium hydroxide and calcium hydroxide.

The reaction of preparation process (e) may be conducted in a substantially wide range of temperature. Generally, the reaction may be carried out at a temperature of from about −20 to about 200° C., preferably from 0 to about 150° C. Further, this reaction is preferably carried out under normal pressure although it may also be operated under increased pressure or reduced pressure.

When the reaction of preparation process (e) is carried out, for example, 1 mole of the compound of the formula (IA) may be reacted with 1 mole or slightly excess amount of a phenylboric acid derivative (VII) in the presence of a catalyst amount of palladium acetate and 1 mole or slightly excess amount of potassium carbonate in a diluent such as water to obtain the objective compound of the corresponding formula (I).

The compounds of the formula (VIII), which are used as a starting material in preparation process (f), are novel and may be obtained by the following process using the compound of formula (XLV), phthalimide potassium well known in the field of organic chemistry and the compound of formula (II) as starting materials:

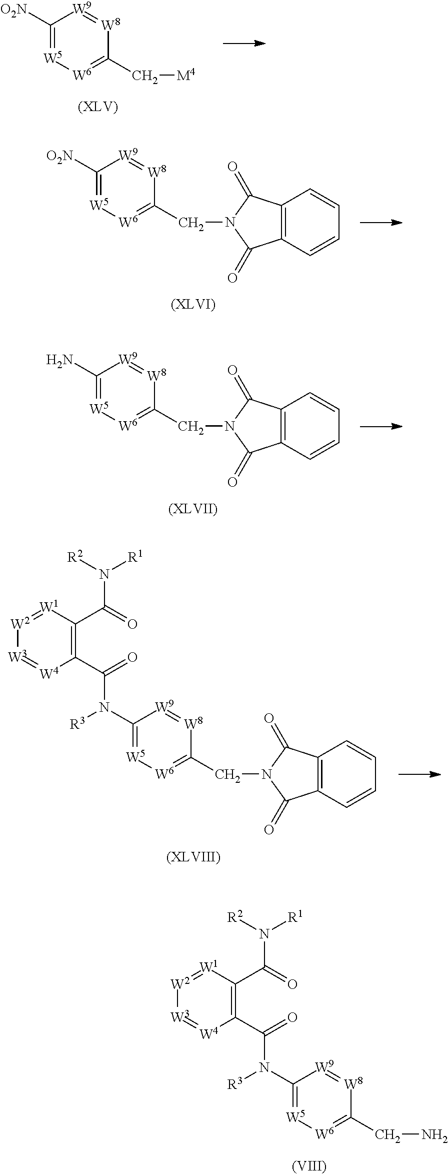

wherein R¹, R², R³, W¹, W², W³, W⁴, W⁵, W⁶, W⁸, W⁹ and M⁴ are as defined herein.

The reaction of the compound of the formula (XLV) with phthalimide potassium may be carried out in an appropriate diluent, and examples of diluents used therefor may include aliphatic, alicyclic and aromatic hydrocarbons such as toluene and chlorobenzene; and acid amides such as dimethylformamide (DMF) and dimethylacetoamide (DMA).

The reaction may be conducted in a substantially wide range of temperature. Generally, the reaction may be carried out at a temperature of from about −20 to about 150° C., preferably from room temperature to about 100° C. Further, this reaction is preferably carried out under normal pressure although it may also be operated under increased pressure or reduced pressure.

When the reaction is carried out, for example, 1 mole of the compound of the formula (VLV) may be reacted with 1 mole or slightly excess mole amount of potassium phthalimide in a diluent such as DMF to obtain the objective compound of the formula (XLVI).

The compounds of (XLVI) include known compounds and specific examples thereof may include: 2-(4-nitrobenzyl)-1H-isoindole-1,3-(2H)-dione, 2-(3-chloro-4-nitrobenzyl)-1H-isoindole-1,3-(2H)-dione, 2-(3-methyl-4-nitrobenzyl)-1H-isoindole-1,3-(2H)— dione and 2-[(5-nitropyridin-2-yl)methyl]-1H-isoindole-1,3-(2H)-dione.

According to a direct hydrogen reduction process or a reducing reaction using metal, which is well known in the field of organic chemistry, the compound of the formula (XLVI) is converted to the compound of the formula (XLVII).

As the reducing process using metal, a process in which tin (II) chloride is reacted under oxidizing conditions (Organic Syntheses Collective, Vol. II, p. 254) may be exemplified.

The process in which tin (II) chloride is reacted under oxidizing conditions may be carried out in an appropriate diluent, and examples of the diluents used therefor may include water and ethanol. The reaction may be carried out in the presence of mineral acids such as hydrochloric acid at a temperature normally from about −20 to 100° C., preferably from 0 to about 80° C. Further, this reaction is preferably carried out under normal pressure although it may also be operated under increased pressure or reduced pressure.

When the reaction is carried out, for example, for example, 1 mole of the compound of the formula (XLVI) may be reacted with 5 to 10 mole of tin (II) chloride in the presence of concentrated hydrochloric acid in a diluent such as ethanol to obtain the objective compound of the formula (XLVII).

The compounds of the formula (XLVII) include known compounds, and specific examples thereof may include: 2-(4-aminobenzyl)-1H-isoindole-1,3(2H)-dione, 2-(3-chloro-4-aminobenzyl)-1H-isoindole-1,3(2H)-dione, 2-(3-methyl-4-aminobenzyl)-1H-isoindole-1,3(2H)-dione and 2-[(5-aminopyridine-2-yl)methyl]-1H-isoindole-1,3(2H)-dione.

The compound of the formula (XLVII) may be reacted with the compound of the formula (II) according to the above preparation process (a), and the compound of the formula (XLVIII) may be obtained.

The compounds of the formula (XLVIII) are novel and specific examples thereof may include the following compounds:

N-{4-[(1,3-dioxo-1,3-dihydro-2H-isoindol-2-yl)methyl]phenyl}-N′-isopropyl-1,2-benzenedicarboxamide, N¹-{4-[(1,3-dioxo-1,3-dihydro-2H-isoindol-2-yl)methyl]-2-methylphenyl}-N²-[(1S)-1-methyl-2-(methylthio)ethyl]-1,2-benzenedicarboxamide, 3-chloro-N′-{4-[(1,3-dioxo-1,3-dihydro-2H-isoindol-2-yl)methyl]-2-methylphenyl}-N²-[(1S)-1-methyl-2-(methylthio)ethyl-1,2-benzenedicarboxamide, 3-chloro-N¹-{4-[(1,3-dioxo-1,3-dihydro-2H-isoindol-2-yl)methyl]-2-chlorophenyl}-N²-[(1S)-1-methyl-2-(methylthio)ethyl-1,2-benzenedicarboxamide and 3-chloro-N¹-{6-[(1,3-dioxo-1,3-dihydro-2H-isoindol-2-yl)methyl]-pyridine-3-yl}-N²-[(1S)-1-methyl-2-(methylthio)ethyl-1,2-benzenedicarboxamide.

The compound of the formula (XLVIII) is converted to the compound of the formula (VIII) according to the process described in J. Chem. Soc., 1926, p. 2348.

When the reaction is carried out, for example, 1 mole of the compound of the formula (XLVIII) may be reacted with 5 to 15 mole amount of hydrazine hydrate in a diluent such as ethanol, to obtain the compound of the formula (VIII).

The compounds of the formula (VIII) are novel and specific examples thereof may include the following compounds: N-[4-(aminomethyl)phenyl]-N′-isopropyl-1,2-benzenedicarboxamide, N-[4-(aminomethyl)-2-methylpheny]-N′-[(1S)-1-methyl-2-(methylthio)ethyl]-1,2-benzenedicarboxamide, N¹-[4-(aminomethyl)-2-methylphenyl]-3-chloro-N²-[(1S)-1-methyl-2-(methylthio)ethyl]-1,2-benzene-dicarboxamide, N′-[4-(aminomethyl)-2-chlorophenyl]-3-chloro-N²-[(1S)-1-methyl-2-(methylthio)ethyl]-1,2-benzene-dicarboxamide, and N-[6-(aminomethyl)pyridine-3-yl]-3-chloroN²-[(1S)-1-methyl-2-(methylthio)ethyl]-1,2-benzene-dicarboxamide.

Many of the compounds of the formula (IX) are known and specific examples thereof may include: 2,5-bis(difluoromethyl)-1,3,4-oxadiazole, 2,5-bis(trifluoromethyl)-1,3,4-oxadiazole, 2,5-bis(pentafluoroethyl)-1,3,4-oxadiazole and 2,5-bis(heptafluoro-n-propyl)-1,3,4-oxadiazole. The reaction of preparation process (f) may be carried out either in the presence of a diluent such as alcohols such as methanol or without solvent.

The reaction of preparation process (f) may be conducted in a substantially wide range of temperature. Generally, the reaction may be carried out at a temperature of from about −50 to about 200° C., preferably from 0 to about 150° C. Further, this reaction is preferably carried out under normal pressure although it may also be operated under increased pressure or reduced pressure.

When the reaction of preparation process (f) is carried out, for example, 1 mole of the compound of the formula (VIII) may be reacted with 1 mole or slightly excess amount of the compound of the formula (IX) in a diluent such as methanol to obtain the objective compound of the corresponding formula (I).

The compounds of the formula (IB), which are used as a starting material in preparation process (g), are novel compounds synthesized according to preparation processes (a), (b) and (c), and specific examples thereof may include the following compounds: benzyl N-({2-[(4-{[3,5-bis(trifluoromethyl)-1H-pyrazol-1-yl]methyl}-2-methylphenyl)carbamoyl]-6-chlorobenzoyl}alaninate, benzyl N-({2-[(4-{[3,5-bis(trifluoromethyl)-1H-1,2,4-triazol-1-yl]methyl}-2-methylphenyl)carbamoyl]-6-chlorobenzoyl}alaninate, benzyl N-({2-[(4-{[3,5-bis(trifluoromethyl)-1H-pyrazol-1-yl]methyl}-2-methylphenyl)carbamoyl]-6-chlorobenzoyl}-2-methylalaninate, and benzyl N-({2-[(4-{[3,5-bis(trifluoromethyl)-1H-1,2,4-triazol-1-yl]methyl}-2-methylphenyl)carbamoyl]-6-chlorobenzoyl}-2-methylalaninate.

In the reaction of preparation process (g), a debenzylating agent such as boron tribromide and hydrogen may be used.

The reaction of preparation process (g) may be carried out in either an appropriate diluent or a combination of appropriate diluents. When boron tribromide is used, examples of the diluents used therefor may include alicyclic and aromatic hydrocarbons (optionally chlorinated) such as dichloromethane. In the case of a catalytic reduction with hydrogen, examples of the diluents may include ethers such as ethyl ether, methyl ethyl ether, isopropyl ether, butyl ether, dioxane and tetrahydrofuran (THF); and alcohols such as methanol, ethanol, isopropanol, butanol and ethylene glycol, and examples of catalytic reduction catalysts may include palladium carbon, Raney nickel and platinum oxide.

The reaction of preparation process (g) may be conducted in a substantially wide range of temperature. Generally, the reaction may be carried out at a temperature of from about −50 to about 200° C., preferably from −20 to about 100° C. Further, this reaction is preferably carried out under normal pressure although it may also be operated under increased pressure or reduced pressure.

When the reaction of preparation process (g) is carried out, for example, 1 mole of the compound of the formula (IB) may be reacted with 1 mole or slightly excess amount of boron tribromide in a diluent such as dichloroethane to obtain the objective compound of the corresponding formula (I).

The compounds of the formula (IC), which are used as a starting material in preparation process (h), are novel compounds which are synthesized according to preparation process (g) and included in the scope of the formula (I). Specific examples of the compounds may include the following compounds: N-({2-[(4-{[3,5-bis(trifluoromethyl)-1H-pyrazol-1-yl]methyl}-2-methylphenyl)carbamoyl]-6-chlorobenzoyl}alanine, N-({2-[(4-{[3,5-bis(trifluoromethyl)-1H-1,2,4-triazol-1-yl]methyl}-2-methylphenyl)carbamoyl]-6-chlorobenzoyl}alanine, N-({2-[(4-[3,5-bis(trifluoromethyl)-1H-pyrazol-1-yl]methyl}-2-methylphenyl)carbamoyl]-6-chlorobenzoyl}-2-methylalanine, and N-({2-[(4-[3,5-bis(trifluoromethyl)-1H-1,2,4-triazol-1-yl]methyl}-2-methylphenyl)carbamoyl]-6-chlorobenzoyl}-2-methylalanine.

Many of the compounds of the formula (X) are known and specific examples thereof may include methylamine, ethylamine, dimethylamine, propargylamine, cyclopropylamine and glycine methyl ester hydrochloride.

The reaction of preparation process (h) may be carried out in an appropriate diluent or a combination of appropriate diluents, and examples of the diluents used therefor may include aliphatic, alicyclic and aromatic hydrocarbons (optionally chlorinated) such as pentane, hexane, cyclohexane, petroleum ether, ligroin benzene, toluene, xylene, dichloromethane, chloroform, carbon tetrachloride, 1,2-dichloroethane, chlorobenzene and dichlorobenzene; ethers such as ethyl ether, methyl ethyl ether, isopropyl ether, butyl ether, dioxane, dimethoxyethane (DME), tetrahydrofuran (THF) and diethylene glycol dimethyl ether (DGM); esters such as ethyl acetate and amyl acetate; and acid amides such as dimethylformamide (DMF), dimethylacetamide (DMA), N-methylpyrrolidone, 1,3-dimethyl-2-imidazolidinone and hexamethylphosphoric triamide (HMPA).

The reaction of preparation process (h) is carried out in the presence of a condensation agent. Examples of the condensation agents may include carbonyldiimidazole, dicyclohexylcarbodiimide and N-(3-dimethylaminopropyl)-N′-ethylcarbodiimide hydrochloride.

The reaction of preparation process (h) may be carried out in the presence of a base, and examples of the bases may include tertiary amines, dialkylaminoanilines and pyridines such as triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO), and 1,8-diazabicyclo[5,4,0]undec-7-ene (DBU).

The reaction of preparation process (h) may be conducted in a substantially wide range of temperature. Generally, the reaction may be carried out at a temperature of from about −50 to about 200° C., preferably from −20 to about 100° C. Further, this reaction is preferably carried out under normal pressure although it may also be operated under increased pressure or reduced pressure.

When the reaction of preparation process (h) is carried out, for example, 1 mole of the compound of the formula (IC) may be reacted with 1 mole or slightly excess amount of (XII) in the presence of 1 mole or slightly excess amount of a condensation agent such as N-(3-dimethylaminopropyl)-N′-ethylcarbodiimide hydrochloride and 1 mole or slightly excess amount of a base such as triethylamine in a diluent such as DMF to obtain the objective compound of the corresponding formula (I).

The compounds of the formula (ID), which are used as a starting material in preparation process (i), are novel compounds synthesized according to preparation processes (a), (b) and (c), and specific examples thereof may include the following compounds: N²-[2-(benzyloxy)-1,1-dimethylethyl]-3-chloro-N¹-(2-methyl-4-{[3,5-bis(trifluoromethyl)-1H-pyrazol-1-yl]methyl}-phenyl)-1,2-benzenedicarboxamide, N²-[2-(benzyloxy)-1,1-dimethylethyl]-3-chloro-N′-(2-methyl-4-{[3,5-bis(trifluoromethyl)-1H-1,2,4-triazol-1-yl]methyl}phenyl)-1,2-benzenedicarboxamide, and N²-[2-(benzyloxy)-1,1-dimethylethyl]-3-chloro-N¹-(2-methyl-4-{[3-(pentafluoroethyl)-5-(trifluoromethyl)-1H-1,2,4-triazol-1-yl]methyl 1 phenyl)-1,2-benzenedicarboxamide.

In the reaction of preparation process (i), boron tribromide, hydrogen and the like may be used as a debenzylating agent.

The reaction of preparation process (i) may be carried out in either an appropriate diluent or a combination of appropriate diluents. When boron tribromide is used, examples of the diluents thereof may include alicyclic and aromatic hydrocarbons (optionally chlorinated) such as dichloromethane. In the case of a catalytic reduction with hydrogen, examples of the diluents may include ethers such as ethyl ether, methyl ethyl ether, isopropyl ether, butyl ether, dioxane and tetrahydrofuran (THF); and alcohols such as methanol, ethanol, isopropanol, butanol and ethylene glycol, and examples of catalytic reduction catalysts may include palladium carbon, Raney nickel and platinum oxide.

The reaction of preparation process (i) may be conducted in a substantially wide range of temperature. Generally, the reaction may be carried out at a temperature of from about −50 to about 200° C., preferably from −20 to about 100° C. Further, this reaction is preferably carried out under normal pressure although it may also be operated under increased pressure or reduced pressure.

When the reaction of preparation process (i) is carried out, for example, 1 mole of the compound of the formula (ID) may be reacted with 1 mole or slightly excess amount of boron tribromide in a diluent such as dichloroethane to obtain the objective compound of the corresponding formula (I).

The compounds of the formula (IE), which are used as a starting material in preparation process (j), are novel compounds which are synthesized according to preparation process (i) and included in the scope of the formula (I). Specific examples of the compounds may include the following known compounds.

3-chloro-N²-(2-hydroxy-1,1-dimethylethyl)-N¹-(2-methyl-4-{[3,5-bis(trifluoromethyl)-1H-pyrazol-1-yl]methyl}phenyl)-1,2-benzenedicarboxamide, 3-chloro-N²-(2-hydroxy-1,1-dimethylethyl)-N′-(2-methyl-4-{[3,5-bis(trifluoromethyl)-1H-1,2,4-triazol-1-yl]methyl}phenyl)-1,2-benzenedicarboxamide, and 3-chloro-N²-(2-hydroxy-1,1-dimethylethyl)-N¹-(2-methyl-4-{[3-(pentafluoroethyl)-5-(trifluoromethyl)-1H-1,2,4-triazol-1-yl]methyl}phenyl)-1,2-benzene-dicarboxamide.

In the reaction of preparation process (j), an oxidizing agent, such as chromium oxide, pyridinium chlorochromate (PCC), pyridinium dichromate (PDC), active manganese dioxide and a mixture of DMSO and oxalyl chloride may be used.

The reaction of preparation process (j) may be carried out in an appropriate diluent or a combination of appropriate diluents, and examples of the diluents used therefor may include aliphatic, alicyclic and aromatic hydrocarbons (optionally chlorinated) such as pentane, hexane, cyclohexane, petroleum ether, dichloromethane, chloroform, carbon tetrachloride, 1,2-dichloroethane, chlorobenzene and dichlorobenzene; ketones such as acetone; and ethers such as diethyl ether.

The reaction of preparation process (j) may be conducted in a substantially wide range of temperature. Generally, the reaction may be carried out at a temperature of from about −70 to about 150° C., preferably from −60 to about 100° C. Further, this reaction is preferably carried out under normal pressure although it may also be operated under increased pressure or reduced pressure.

When the reaction of preparation process (j) is carried out, for example, 1 mole of the compound of the formula (IE) may be reacted with 1 mole or slightly excess amount of the mixture of DMSO and oxalyl chloride as an oxidizing agent in a diluent such as dichloromethane to obtain the objective compound of the corresponding formula (I).

The compounds of the formula (IF), which are used as a starting material in preparation process (k), are novel compounds which are synthesized according to preparation process (j) and included in the scope of the formula (I). Specific examples thereof may include the following compounds.

3-chloro-N²-(1,1-dimethyl-2-oxoethyl)-N′-(2-methyl-4-{[3,5-bis(trifluoromethyl)-1H-pyrazol-1-yl]methyl}phenyl)-1,2-benzenedicarboxamide, 3-chloro-N²-(1,1-dimethyl-2-oxoethyl)-N¹-(2-methyl-4-{[3,5-bis(trifluoromethyl)-1H-1,2,4-triazol-1-yl]methyl}phenyl)-1,2-benzenedicarboxamide, and 3-chloro-N²-(1,1-dimethyl-2-oxoethyl)-N′-(2-methyl-4-{[3-(pentafluoroethyl)-5-(trifluoromethyl)-1H-1,2,4-triazol-1-yl]methyl}phenyl)-1,2-benzenedicarboxamide.

Many of the compounds of the formula (XI) are known, and specific examples thereof may include hydroxylamine hydrochloride and O-methylhydroxylammonium chloride.

The reaction of preparation process (k) may be carried out in an appropriate diluent, and examples of the diluents used therefor may include pyridine, ethanol, isopropanol and water.

The reaction of preparation process (k) is carried out in the presence of a base, and examples of bases may include inorganic bases such as sodium acetate and organic bases such as pyridine and triethylamine.

The reaction of preparation process (k) may be conducted in a substantially wide range of temperature. Generally, the reaction may be carried out at a temperature of from about 0 to about 200° C., preferably from room temperature to about 100° C. Further, this reaction is preferably carried out under normal pressure although it may also be operated under increased pressure or reduced pressure.

When the reaction of preparation process (k) is carried out, for example, 1 mole of the compound of the formula (IF) may be reacted with 1 mole or slightly excess mole amount of the compound of the formula (XI) in a diluent such as ethanol in the presence of 1 mole or slightly excess mole amount of sodium acetate as an inorganic base to obtain the objective compound of the corresponding formula (I).

The compounds of the formula (IG), which are used as a starting material in preparation process (l), are novel compounds that are synthesized according to the above preparation processes (a), (b), (c), (d), (e) and (f) and included in the scope of the formula (I) of the present invention. Specific examples thereof may include the following compounds.

N-(4-{[3,5-bis(trifluoromethyl)-1H-pyrazol-1-yl]methyl}-2-methylphenyl)-N′-[(1S)-1-methyl-2-(methylthio)ethyl]-1,2-benzenedicarboxamide, N-(4-{[3,4-bis(pentafluoroethyl)-1H-pyrazol-1-yl]methyl}-2-methylphenyl)-N′-[(1S)-1-methyl-2-(methylthio)ethyl]-1,2-benzenedicarboxamide, N-(4-{[3,5-bis(trifluoromethyl)-1H-1,2,4-triazol-1-yl]methyl}-2-methylphenyl)-N′-[(1S)-1-methyl-2-(methylthio)ethyl]-1,2-benzenedicarboxamide, N-[4-(4-{[3,5-bis(trifluoromethyl)phenyl]-5-oxo-4,5-dihydro-1H-tetrazol-1-yl]methyl)-2-methylphenyl)-N′-[(1S)-1-methyl-2-(methylthio)ethyl]-1,2-benzenedicarboxamide, N′-(4-{[3,5-bis(trifluoromethyl)-1H-pyrazol-1-yl]methyl}-2-methylphenyl)-N²-[(1S)-1-methyl-2-(methylthio)ethyl]-3-(trifluoromethoxy)-1,2-benzenedicarboxamide, N¹-(4-{[3,5-bis(trifluoromethyl)-1H-pyrazol-1-yl]methyl}-2,6-dichlorophenyl)-3-chloro-N²-[(1S)-1-methyl-2-(methylthio)ethyl]-1,2-benzenedicarboxamide, N²-(6-{[3,4-bis(pentafluoroethyl)-1H-pyrazol-1-yl]methyl}-2-methylpyridine-3-yl)-3-chloro-N²-[(1S)-1-methyl-2-(methylthio)ethyl]-1,2-benzenedicarboxamide, N′-(4-{[3,5-bis(trifluoromethyl)-1H-1,2,4-triazol-1-yl]methyl}-2,6-dichlorophenyl)-3-chloro-N²-[(1S)-1-methyl-2-(methylthio)ethyl]-1,2-benzenedicarboxamide, 3-chloro-N²-[(1S)-2-(ethylthio)-1-methylethyl]-N¹-(2-methyl-4-{[5-(pentafluoroethyl)-3-(trifluoromethyl)-1H-1,2,4-triazol-1-yl]methyl}-1,2-benzenedicarboxamide, N′-(4-{[3,5-bis(1,1,2,2-tetrafluoroethyl)-1H-1,2,4-triazol-1-yl]methyl}-2-methylphenyl)-3-chloro-N²-[(1S)-2-(ethylthio)-1-methylethyl]-1,2-benzenedicarboxamide, 3-chloro-N²-[1,1-dimethyl-2-(methylthio)ethyl]-N¹— (2-methyl-4-{5-(pentafluoroethyl)-3-(trifluoromethyl)-1H-1,2,4-triazol-1-yl]methyl}phenyl)-1,2-benzenedicarboxamide, 3-chloro-N′-[4-({5-[chloro(difluoro)methyl]-3-(pentafluoroethyl)-1H-1,2,4-triazol-1-yl]methyl}-2-methylethyl]-N²-[1,1-dimethyl-2-(methylthio)ethyl])-1,2-benzenedicarboxamide, N¹-(4-{[3,5-bis(trifluoromethyl)-1H-1,2,4-triazol-1-yl]methyl}-2-methylphenyl)-3-chloro-N²-[(1S)-2-(ethylthio)-1,1-dimethylethyl]-1,2-benzenedicarboxamide, and N¹-(4-{[3,4-bis(pentafluoroethyl)-1H-pyrazol-1-yl]methyl}-2-methylphenyl)-3-bromo-N²-[(1S)-1-methyl-2-(methylthio)ethyl]-1,2-benzenedicarboxamide.

The reaction of preparation process (l) may be carried out in an appropriate diluent, and examples of the diluents used therefor may include aliphatic, alicyclic and aromatic hydrocarbons

(optionally chlorinated) such as benzene, toluene, xylene, dichloromethane, chloroform, carbon tetrachloride, 1,2-dichloroethane, chlorobenzene and dichlorobenzene; alcohols such as methanol, ethanol, isopropanol and butanol; and acids such as formic acid and acetic acid.

Examples of oxidizing agents which may be used in the reaction of preparation process (1) may include metachloroperbenzoic acid, peracetic acid, potassium metaperiodate, potassium hydrogen persulfate (oxone) and hydrogen peroxide.

The reaction of preparation process (1) may be conducted in a substantially wide range of temperature. Generally, the reaction may be carried out at a temperature of from about −50 to about 150° C., preferably from −10 to about 100° C. Further, this reaction is preferably carried out under normal pressure although it may also be operated under increased pressure or reduced pressure.

When the reaction of preparation process (1) is carried out, for example, 1 mole of the compound of the formula (IG) may be reacted with 1 to 5 mole of an oxidizing agent such as metachloroperbenzoic acid in a diluent such as dichloromethane to obtain the objective compound of a corresponding formula (I).

The compounds of the formula (IH), which are used as a starting material in preparation process (m), are novel compounds synthesized according to the above preparation processes (a), (b) and (c), and specific examples thereof may include the following compounds: N¹-(4-{[3,5-bis(trifluoromethyl)-1H-pyrazol-1-yl]acetyl}-2-methylphenyl)-3-chloro-N²-[(1S)-1-methyl-2-(methylthio)ethyl]-1,2-benzenedicarboxamide and N′-(4-{[3,5-bis(trifluoromethyl)-1H-1,2,4-triazol-1-yl]acetyl}-2-methylphenyl)-3-methylthio-N²-[(1S)-1-methyl-2-(methylthio)ethyl]-1,2- The reaction of preparation process (m) may be carried out in an appropriate diluent, and examples of the diluents used therefor may include ethers such as THF; and alcohols such as methanol, ethanol, isopropanol and butanol.

Examples of the reducing agents which may be used in the reaction of preparation process (m) may include lithium/aluminum tri-t-butoxide, borane dimethyl sulfide complex and sodium borohydride.

The reaction of preparation process (m) may be conducted in a substantially wide range of temperature. Generally, the reaction may be carried out at a temperature of from about −50 to about 150° C., preferably from −20 to about 100° C. Further, this reaction is preferably carried out under normal pressure although it may also be operated under increased pressure or reduced pressure.

When the reaction of preparation process (m) is carried out, for example, 1 mole of the compound of the formula (III) may be reacted with 1 to 5 mole of a reducing agent such as sodium borohydride in a diluent such as methanol to obtain the objective compound of the corresponding formula (I).

The compounds of the formula (IJ), which are used as a starting material in preparation process (n), are novel compounds synthesized according to the above preparation processes (a), (b) and (c), and specific examples thereof may include the following compounds: 3-chloro-N′-(2-methyl-4-{[3-(trifluoromethyl)-5-(trifluoromethyl)-4,5-dihydro-1H-1,2,4-triazol-1-yl]methyl}phenyl-N²-[(1S)-1-methyl-2-(methylthio)ethyl]-1,2-benzenedicarboxamide, 3-methylthio-N′-(2-methyl-4-{[3-(trifluoromethyl)-5-(trifluoromethyl)-4,5-dihydro-1H-1,2,4-triazol-1-yl]methyl}phenyl-N²-[(1S)-1-methyl-2-(methylthio)ethyl]-1,2-benzenedicarboxamide, 3-chloro-N′-(2-methyl-4-{[3-(pentafluoroethyl)-5-(trifluoromethyl)-4,5-dihydro-1H-1,2,4-triazol-1-yl]methyl}phenyl-N²-[(1S)-1-methyl-2-(methylthio)ethyl]-1,2-benzenedicarboxamide, 3-methylthio-N′-(2-methyl-4-{[3-(pentafluoroethyl)-5-(trifluoromethyl)-4,5-dihydro-1H-1,2,4-triazol-1-yl]methyl}phenyl)-N²-[(1S)-1-methyl-2-(methylthio)ethyl]-1,2-benzenedicarboxamide, and 3-chloro-N′-(4-{[5,5-dimethyl-3-(pentafluoroethyl)-4,5-dihydro-1H-1,2,4-triazol-1-yl]methyl}-2-methylphenyl)-N²-[(1S)-1-methyl-2-(methylthio)ethyl]-1,2-benzenedicarboxamide.

The compounds of the formula (XII), which are used as a starting material in preparation process (n), are compounds well known in the field of organic chemistry, and examples thereof may include commercially available acetyl chloride, propionyl chloride, 2,2,3,3-tetrafluoropropionyl chloride, heptafluorobutyloyl chloride, chloromethyl carbonate and chloroethyl carbonate. Further, the compounds of the formula (XIII) are compounds well known in the field of organic chemistry, and examples thereof may include commercially available acetic anhydride, propionic anhydride, difluoroacetic anhydride, trifluoroacetic anhydride, chlorodifluoroacetic anhydride, pentafluoropropionic anhydride, heptafluorobutyric anhydride and di-t-butyl bicarbonate.

A reaction of the compound of the formula (IJ) with the compound of the formula (XII) or (XIII) may be carried out in an appropriate diluent and examples of the diluents used therefor may include water; aliphatic, alicyclic and aromatic hydrocarbons (optionally chlorinated) such as pentane, hexane, cyclohexane, petroleum ether, ligroin, benzene, toluene, xylene and dichloromethane; and ethers such as ethyl ether, methyl ethyl ether, isopropyl ether, butyl ether, dioxane, dimethoxyethane (DME), tetrahydrofuran (THF) and diethylene glycol dimethyl ether (DGM).

The reaction of the compound of the formula (IJ) with the compound of the formula (XII) or (XIII) may be conducted in a substantially wide range of temperature. Generally, the reaction may be carried out at a temperature of from about −20 to about 200° C., preferably from 0 to about 150° C. Further, this reaction is preferably carried out under normal pressure although it may also be operated under increased pressure or reduced pressure.

The reaction of the compound of the formula (D) with the compound of the formula (XII) is carried out in the presence of a base, and examples of the bases may include tertiary amines, dialkylaminoanilines and pyridines such as triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO) and 1,8-diazabicyclo[5,4,0]undec-7-ene (DBU); and inorganic bases including hydroxides, carbonates and bicarbonates of alkali metals or alkaline earth metals such as sodium hydrogen carbonate, potassium hydrogen carbonate, sodium carbonate, potassium carbonate, lithium hydroxide, sodium hydroxide and potassium hydroxide.

When the reaction of the compound of the formula (IJ) with the compound of the formula (XII) or (XIII) is carried out, for example, 1 mole of the compound of the formula (IJ) may be reacted with 1 mole or slightly excess amount of the compound of the formula (XIII) in a diluent such as THF to obtain the objective compound of the corresponding formula (I).

The compounds of the formula (IK), which are used as a starting material in preparation process (o), are novel compounds that are synthesized according to the above preparation processes (a), (b) and (c) and included in the scope of the formula (I) of the present invention, and specific examples thereof may include the following compounds: 3-iodo-N¹-(2-methyl-4-{[4-(trifluoroacetyl)-3-(trifluoromethyl)-1H-pyrazol-1-yl]methyl}phenyl)-N²-[(1S)-1-methyl-2-(methylthio)ethyl]-1,2-benzenedicarboxamide, 3-chloro-N¹-(2-methyl-4-[4-(trifluoroacetyl)-3-(trifluoromethyl)-1H-pyrazol-1-yl]methyl}phenyl)-N²-[(1S)-1-methyl-2-(methylthio)ethyl]-1,2-benzenedicarboxamide, 3-chloro-N′-(2-methyl-4-{[3-(pentafluoroethyl)-5-(trifluoroacetyl)-1H-pyrazol-1-yl]methyl phenyl)-N²-[(1S)-1-methyl-2-(methylthio)ethyl]-1,2-benzenedicarboxamide, and N^{I}-(2-methyl-4-{[3-(pentafluoroethyl)-5-(trifluoroacetyl)-1H-pyrazol-1-yl]methyl}phenyl)-3-(methylthio)-N²-[(1S)-1-methyl-2-(methylthio)ethyl]-1,2-benzenedicarboxamide.

The compounds of the formula (XI) are as mentioned herein.

The reaction of preparation process (o) may be carried out in an appropriate diluent, and examples of the diluents used therefor may include pyridine, ethanol, isopropanol and water.

The reaction of preparation process (o) may be carried out in the presence of a base, and examples of the bases may include sodium acetate as an inorganic base and pyridine and triethylamine as an organic base.

The reaction of preparation process (o) may be conducted in a substantially wide range of temperature. Generally, the reaction may be carried out at a temperature from about 0 to about 200° C., preferably from room temperature to about 100° C. Further, this reaction is preferably carried out under normal pressure although it may also be operated under increased pressure or reduced pressure.

When the reaction of preparation process (o) is carried out, for example, 1 mole of the compound of the formula (IK) may be reacted with 1 mole or slightly excess mole amount of the compound of the formula (XI) in a diluent such as a mixture solvent of pyridine and ethanol to obtain the objective compound of the corresponding formula (I).

The compounds of formula (I) of the present invention have a potent insecticidal action. Therefore, the compounds of formula (I) of the present invention can be used as an insecticide. Further, the active compounds of formula (I) of the present invention exhibits an infallible control effect against harmful insects, without imposing any harmful side effects of drug to cultivated plants. The compound of the present invention can be used for the control of a wide range of pest species, for example, harmful sucking insects, chewing insects, as well as other plant parasitic pests, storage insects, hygiene pests and the like, and can be applied for the purpose of disinfestation and extermination thereof.

Examples of such harmful insects include the harmful insects as shown in the following.

As for insects, coleopterans, for example, Callosobruchus chinensis, Sitophilus zeamais, Tribolium castaneum, Epilachna vigintioctomaculata, Agriotes fuscicollis, Anomala rufocuprea, Leptinotarsa decemlineata, Diabrotica spp., Monochamus alternatus, Lissorhoptrus oryzophilus, Lyctus bruneus, Aulacophora femoralis;

lepidopterans, for example, Lymantria dispar, Malacosoma neustria), Pieris rapae, Spodoptera litura, Mamestra brassicae, Chilo suppressalis), Pyrausta nubilalis, Ephestia cautella, Adoxophyes orana, Carpocapsa pomonella, Agrotisfucosa, Galleria mellonella, Plutella maculipennis, Heliothis virescens, Phyllocnistis citrella;
hemipterans, for example, Nephotettix cincticeps, Nilaparvata lugens, Pseudococcus comstocki, Unaspis yanonensis, Myzus persicas, Aphis pomi, Aphis gossypii, Rhopalosiphum pseudobrassicas, Stephanitis nashi, Nezara spp., Trialeurodes vaporariorm, Psylla spp.;
thysanopterans, for example, Thrips palmi, Franklinella occidental;
orthopterans, for example, Blatella germanica, Periplaneta americana, Gryllotalpa Africana, Locusta migratoria migratoriodes;
isopterans, for example, Reticulitermes speratus, Coptotermes formosanus;
dipterans, for example, Musca domestica, Aedes aegypti, Hylemia platura, Culex pipiens, Anopheles sinensis, Culex tritaeniorhynchus, Liriomyza trifolii; and the like may be mentioned.

As for acari, for example, Tetranychus cinnabarinus, Tetranychus urticae, Panonychus citri, Aculops pelekassi, Tarsonemus spp.), and the like may be mentioned.

As for nematodes, for example, Meloidogyne incognita, Bursaphelenchus lignicolus Mamiya et Kiyohara, Aphelenchoides besseyi, Heterodera glycines, Pratylenchus spp., and the like may be mentioned.

Furthermore, in the field of veterinary medicine, the novel compounds of the present invention can be effectively used against various harmful animal parasitic pests (endoparasites and ectoparasites), for example, insects and helminthes.

Examples of the insects include Gasterophilus spp., Stomoxys spp., Trichodectes spp., Rhodnius spp., Ctenocephalides canis, Cimx lecturius, Ctenocephalides felis, Lucilia cuprina, and the like.

Examples of acari include Ornithodoros spp., Ixodes spp., Boophilus spp., and the like.

The compounds according to the present invention show a potent insecticidal action and can therefore be used as an insecticide. Furthermore, the compounds according to the present invention exhibit a strong control effect against harmful insects, without imposing any harmful side effects of drug to cultivated plants. The compounds of the present invention can thus be used for the control of a wide range of pest species, for example, harmful sucking insects, chewing insects, as well as other plant parasitic pests, storage insects, hygiene pests and the like, and can be applied for the purpose of disinfestation and extermination thereof. Harmful animal pest are for example:

As for insects, coleopterans, for example, Callosobruchus chinensis, Sitophilus zeamais, Tribolium castaneum, Epilachna vigintioctomaculata, Agriotes fuscicollis, Anomala rufocuprea, Leptinotarsa decemlineata, Diabrotica spp., Monochamus alternatus, Lissorhoptrus oryzophilus, Lyctus bruneus, Aulacophora femoralis; lepidopterans, for example, Lymantria dispar, Malacosoma neustria), Pieris rapae, Spodoptera litura, Mamestra brassicae, Chilo suppressalis), Pyrausta nubilalis, Ephestia cautella, Adoxophyes orana, Carpocapsa pomonella, Agrotisfucosa, Galleria mellonella, Plutella maculipennis, Heliothis virescens, Phyllocnistis citrella; hemipterans, for example, Nephotettix cincticeps, Nilaparvata lugens, Pseudococcus comstocki, Unaspis yanonensis, Myzus persicas, Aphis pomi, Aphis gossypii, Rhopalosiphum pseudobrassicas, Stephanitis nashi, Nezara spp., Trialeurodes vaporariorm, Psylla spp.; thysanopterans, for example, Thrips palmi, Franklinella occidental; orthopterans, for example, Blatella germanica, Periplaneta americana, Gryllotalpa Africana, Locusta migratoria migratoriodes; isopterans, for example, Reticulitermes speratus, Coptotermes formosanus; dipterans, for example, Musca domestica, Aedes aegypti, Hylemia platura, Culex pipiens, Anopheles sinensis, Culex tritaeniorhynchus, Liriomyza trifolii.

As for acari, for example, Tetranychus cinnabarinus, Tetranychus urticae, Panonychus citri, Aculops pelekassi, Tarsonemus spp.

As for nematodes, for example, Meloidogyne incognita, Bursaphelenchus lignicolus Mamiya et Kiyohara, Aphelenchoides besseyi, Heterodera glycines, Pratylenchus spp.

Additionally, the compounds according to the present invention show a good plant tolerance and favourable toxicity to warm-blooded animals and being tolerated well by the environment, and thus are suitable for protecting plants and plant parts. Application of the compounds of the invention may result in increasing the harvest yields, improving the quality of the harvested material.

As mentioned before, the compounds can be used for controlling animal pests, in particular insects, arachnids, helminths, nematodes and molluscs, which are encountered in agriculture, in horticulture, the field of veterinary medicine, in forests, in gardens and leisure facilities, in the protection of stored products and of materials, and in the hygiene sector. They may be preferably employed as plant protection agents. They are active against normally sensitive and resistant species and against all or some stages of development. Besides above mentioned pests, such pests include inter alia:

From the order of the Anoplura (Phthiraptera), for example, Damalinia spp., Haematopinus spp., Linognathus spp., Pediculus spp., Trichodectes spp.

From the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example, Dreissena spp.

From the order of the Chilopoda, for example, Geophilus spp., Scutigera spp.

From the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Steprnechus spp., Symphyletes spp., Tenebrio molitor, Tribolium spp., Trogoderma spp., Tychius spp., Xylotrechus spp., Zabrus spp.

From the order of the Collembola, for example, Onychiurus armatus.

From the order of the Dermaptera, for example, Forficula auricularia.

From the order of the Diplopoda, for example, Blaniulus guttulatus.

From the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.

From the class of the Gastropoda, for example, Arion spp., Biomphalaria spp., Bulinus spp., Deroceras spp., Galba spp., Lymnaea spp., Oncomelania spp., Succinea spp.

From the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

It is furthermore possible to control protozoa, such as Eimeria.

From the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccusspp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example, Diprion spp., Hoplocampa spp., Lasius spp., Monomorium pharaonis, Vespa spp.

From the order of the Isopoda, for example, Armadillidium vulgare, Oniscus asellus, Porcellio scaber.

From the order of the Isoptera, for example, Reticulitermes spp., Odontotermes spp.

From the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example, Ceratophyllus spp., Xenopsylla cheopis.

From the order of the Symphyla, for example, Scutigerella immaculata.

From the order of the Thysanoptera, for example, Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.

From the order of the Thysanura, for example, Lepisma saccharina.

The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on their surroundings, habitat or storage space by the customary treatment methods, for example by immersion, spraying, evaporation, fogging, scattering, painting on, injecting and, in the case of propagation material, in particular in the case of seed, also by applying one or more coats.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetically Modified Organisms), and parts thereof, are treated. The terms “parts”, “parts of plants” and “plant parts” have been explained above.

Particularly preferably, plants of the plant cultivars which are in each case commercially available or in use are treated according to the invention. Plant cultivars are understood as meaning plants having novel properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive “synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The preferred transgenic plants or plant cultivars (obtained by genetic engineering) which are to be treated according to the invention include all plants which, by virtue of the genetic modification, received genetic material which imparts particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defense of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, sugar beet, tomatoes, peas and other vegetable varieties, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized in particular are the increased defence of the plants against insects, arachnids, nematodes and slugs and snails by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to hereinbelow as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya beans), Liberty Link® (tolerance to phosphinotricin, for example oilseed rape), IMI® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plant cultivars will be developed and/or marketed in the future.

The plants listed can be treated according to the invention in a particularly advantageous manner with the compounds according to the invention at a suitable concentration.

Furthermore, in the field of veterinary medicine, the novel compounds of the present invention can be effectively used against various harmful animal parasitic pests (endoparasites and ectoparasites), for example, insects and helminthes. Examples of such animal parasitic pests include the pests as described below. Examples of the insects include Gasterophilus spp., Stomoxys spp., Trichodectes spp., Rhodnius spp., Ctenocephalides canis, Cimx lecturius, Ctenocephalides felis, Lucilia cuprina, and the like. Examples of acari include Ornithodoros spp., Ixodes spp., Boophilus spp., and the like.

In the veterinary fields, i.e. in the field of veterinary medicine, the active compounds according to the present invention are active against animal parasites, in particular ectoparasites or endoparasites. The term endoparasites includes in particular helminths, such as cestodes, nematodes or trematodes, and protozoae, such as coccidia. Ectoparasites are typically and preferably arthropods, in particular insects such as flies (stinging and licking), parasitic fly larvae, lice, hair lice, bird lice, fleas and the like; or acarids, such as ticks, for examples hard ticks or soft ticks, or mites, such as scab mites, harvest mites, bird mites and the like.

These parasites include:

From the order of the Anoplurida, for example Haematopinus spp., Linognathus spp., Pediculus spp., Phtirus spp., Solenopotes spp.; particular examples are: Linognathus setosus, Linognathus vituli, Linognathus ovillus, Linognathus oviformis, Linognathus pedalis, Linognathus stenopsis, Haematopinus asini macrocephalus, Haematopinus eurysternus, Haematopinus suis, Pediculus humanus capitis, Pediculus humanus corporis, Phylloera vastatrix, Phthirus pubis, Solenopotes capillatus;

from the order of the Mallophagida and the suborders Amblycerina and Ischnocerina, for example Trimenopon spp., Menopon spp., Trinoton spp., Bovicola spp., Werneckiella spp., Lepikentron spp., Damalina spp., Trichodectes spp., Felicola spp.; particular examples are: Bovicola bovis, Bovicola ovis, Bovicola limbata, Damalina bovis, Trichodectes canis, Felicola subrostratus, Bovicola caprae, Lepikentron ovis, Werneckiella equi;
from the order of the Diptera and the suborders Nematocerina and Brachycerina, for example Aedes spp., Anopheles spp., Culex spp., Simulium spp., Eusimulium spp., Phlebotomus spp., Lutzomyia spp., Culicoides spp., Chrysops spp., Odagmia spp., Wilhelmia spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp., Rhinoestrus spp., Tipula spp.; particular examples are: Aedes aegypti, Aedes albopictus, Aedes taeniorhynchus, Anopheles gambiae, Anopheles maculipennis, Calliphora erythrocephala, Chrysozona pluvialis, Culex quinquefasciatus, Culex pipiens, Culex tarsalis, Fannia canicularis, Sarcophaga carnaria, Stomoxys calcitrans, Tipula paludosa, Lucilia cuprina, Lucilia sericata, Simulium reptans, Phlebotomus papatasi, Phlebotomus longipalpis, Odagmia omata, Wilhelmia equina, Boophthora erythrocephala, Tabanus bromius, Tabanus spodopterus, Tabanus atratus, Tabanus sudeticus, Hybomitra ciurea, Chrysops caecutiens, Chrysops relictus, Haematopota pluvialis, Haematopota italica, Musca autumnalis, Musca domestica, Haematobia irritans irritans, Haematobia irritans exigua, Haematobia stimulans, Hydrotaea irritans, Hydrotaea albipuncta, Chrysomya chloropyga, Chrysomya bezziana, Oestrus ovis, Hypoderma bovis, Hypoderma lineatum, Przhevalskiana silenus, Dermatobia hominis, Melophagus ovinus, Lipoptena capreoli, Lipoptena cervi, Hippobosca variegata, Hippobosca equina, Gasterophilus intestinalis, Gasterophilus haemorroidalis, Gasterophilus inermis, Gasterophilus nasalis, Gasterophilus nigricomis, Gasterophilus pecorum, Braula coeca;
from the order of the Siphonapterida, for example Pulex spp., Ctenocephalides spp., Tunga spp., Xenopsylla spp., Ceratophyllus spp.; particular examples are: Ctenocephalides canis, Ctenocephalides felis, Pulex irritans, Tunga penetrans, Xenopsylla cheopis;
from the order of the Heteropterida, for example Cimex spp., Triatoma spp., Rhodnius spp., Panstrongylus spp.

From the order of the Blattarida, for example Blatta orientalis, Periplaneta americana, Blattela germanica, Supella spp. (e.g. Suppella longipalpa);

From the subclass of the Acari (Acarina) and the orders of the Meta- and Mesostigmata, for example Argas spp., Ornithodorus spp., Otobius spp., Ixodes spp., Amblyomma spp., Rhipicephalus (Boophilus) spp Dermacentor spp., Haemophysalis spp., Hyalomma spp., Dermanyssus spp., Rhipicephalus spp. (the original genus of multi host ticks) Ornithonyssus spp., Pneumonyssus spp., Raillietia spp., Pneumonyssus spp., Stemostoma spp., Varroa spp., Acarapis spp.; particular examples are: Argas persicus, Argas reflexus, Ornithodorus moubata, Otobius megnini, Rhipicephalus (Boophilus) microplus, Rhipicephalus (Boophilus) decoloratus, Rhipicephalus (Boophilus) annulatus, Rhipicephalus (Boophilus) calceratus, Hyalomma anatolicum, Hyalomma aegypticum, Hyalomma marginatum, Hyalomma transiens, Rhipicephalus evertsi, Ixodes ricinus, Ixodes hexagonus, Ixodes canisuga, Ixodes pilosus, Ixodes rubicundus, Ixodes scapularis, Ixodes holocyclus, Haemaphysalis concinna, Haemaphysalis punctata, Haemaphysalis cinnabarina, Haemaphysalis otophila, Haemaphysalis leachi, Haemaphysalis longicorni, Dermacentor marginatus, Dermacentor reticulatus, Dermacentor pictus, Dermacentor albipictus, Dermacentor andersoni, Dermacentor variabilis, Hyalomma mauritanicum, Rhipicephalus sanguineus, Rhipicephalus bursa, Rhipicephalus appendiculatus, Rhipicephalus capensis, Rhipicephalus turanicus, Rhipicephalus zambeziensis, Amblyomma americanum, Amblyomma variegatum, Amblyomma maculatum, Amblyomma hebraeum, Amblyomma cajennense, Dermanyssus gallinae, Ornithonyssus bursa, Ornithonyssus sylviarum, Varroa jacobsoni;

from the order of the Actinedida (Prostigmata) and Acaridida (Astigmata), for example Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.; particular examples are: Cheyletiella yasguri, Cheyletiella blakei, Demodex canis, Demodex bovis, Demodex ovis, Demodex caprae, Demodex equi, Demodex caballi, Demodex suis, Neotrombicula autumnalis, Neotrombicula desaleri, Neöschongastia xerothermobia, Trombicula akamushi, Otodectes cynotis, Notoedres cati, Sarcoptis canis, Sarcoptes bovis, Sarcoptes ovis, Sarcoptes rupicaprae (=S. caprae), Sarcoptes equi, Sarcoptes suis, Psoroptes ovis, Psoroptes cuniculi, Psoroptes equi, Chorioptes bovis, Psoergates ovis, Pneumonyssoidic mange, Pneumonyssoides caninum, Acarapis woodi.

The active compounds according to the invention are also suitable for controlling arthropods, helminths and protozoae, which attack animals. Animals include agricultural livestock such as, for example, cattle, sheep, goats, horses, pigs, donkeys, camels, buffaloes, rabbits, chickens, turkeys, ducks, geese, cultured fish, honeybees. Moreover, animals include domestic animals—also referred to as companion animals—such as, for example, dogs, cats, cage birds, aquarium fish and what are known as experimental animals such as, for example, hamsters, guinea pigs, rats and mice.

By controlling these arthropods, helminths and/or protozoae, it is intended to reduce deaths and improve performance (in the case of meat, milk, wool, hides, eggs, honey and the like) and health of the host animal, so that more economical and simpler animal keeping is made possible by the use of the active compounds according to the invention.

For example, it is desirable to prevent or interrupt the uptake of blood by the parasites from the hosts (when applicable). Also, controlling the parasites may help to prevent the transmittance of infectious agents.

The term “controlling” as used herein with regard to the veterinary field, means that the active compounds are effective in reducing the incidence of the respective parasite in an animal infected with such parasites to innocuous levels. More specifically, “controlling”, as used herein, means that the active compound is effective in killing the respective parasite, inhibiting its growth, or inhibiting its proliferation.

Generally, when used for the treatment of animals the active compounds according to the invention can be applied directly. Preferably they are applied as pharmaceutical compositions which may contain pharmaceutically acceptable excipients and/or auxiliaries which are known in the art.

In the veterinary field and in animal keeping, the active compounds are applied (=administered) in the known manner by enteral administration in the form of, for example, tablets, capsules, drinks, drenches, granules, pastes, boluses, the feed-through method, suppositories; by parenteral administration, such as, for example, by injections (intramuscular, subcutaneous, intravenous, intraperitoneal and the like), implants, by nasal application, by dermal application in the form of, for example, bathing or dipping, spraying, pouring-on and spotting-on, washing, dusting, and with the aid of active-compound-comprising shaped articles such as collars, ear tags, tail tags, limb bands, halters, marking devices and the like. The active compounds may be formulated as shampoo or as suitable formulations usable in aerosols, unpressurized sprays, for example pump sprays and atomizer sprays.

When used for livestock, poultry, domestic animals and the like, the active compounds according to the invention can be applied as formulations (for example powders, wettable powders [“WP”], emulsions, emulsifiable concentrates [“EC”], flowables, homogeneous solutions, and suspension concentrates [“SC”]) which comprise the active compounds in an amount of from 1 to 80% by weight, either directly or after dilution (e.g. 100- to 10 000-fold dilution), or else as a chemical bath.

When used in the veterinary field the active compounds according to the invention may be used in combination with suitable synergists or other active compounds, such as for example, acaricides, insecticides, anthelmintics, anti-protozoal drugs.

In the present invention, a substance having an insecticidal action against pests including all of these is referred to as an insecticide.

When used as an insecticide An active compound of the present invention can be prepared in conventional formulation forms. Examples of the formulation forms include solutions, emulsions, wettable powders, water dispersible granules, suspensions, powders, foams, pastes, tablets, granules, aerosols, active compound-infiltrated natural and synthetic materials, microcapsules, seed coating agents, formulations used with a combustion apparatus (for example, fumigation and smoking cartridges, cans, coils or the like as the combustion apparatus), ULV (cold mist, warm mist), and the like.

These formulations can be produced by methods that are known per se. For example, a formulation can be produced by mixing the active compound with a developer, that is, a liquid diluent or carrier; a liquefied gas diluent or carrier; a solid diluent or carrier, and optionally with a surfactant, that is, an emulsifier and/or dispersant and/or foaming agent.

In the case where water is used as the developer, for example, an organic solvent can also be used as an auxiliary solvent.

Examples of the liquid diluent or carrier include aromatic hydrocarbons (for example, xylene, toluene, alkylnaphthalene and the like), chlorinated aromatic or chlorinated aliphatic hydrocarbons (for example, chlorobenzenes, ethylene chlorides, methylene chlorides), aliphatic hydrocarbons (for example, cyclohexanes), paraffins (for example, mineral oil fractions), alcohols (for example, butanol, glycols and their ethers, esters and the like), ketones (for example, acetone, methyl ethyl ketone, methyl isobutyl ketone, cyclohexanone and the like), strongly polar solvents (for example, dimethylformamide, dimethylsulfoxide and the like), water and the like

The liquefied gas diluent or carrier may be those which are gaseous at normal temperature and normal pressure, for example, aerosol propellants such as butane, propane, nitrogen gas, carbon dioxide and halogenated hydrocarbons.

Examples of the solid diluent include pulverized natural minerals (for example, kaolin, clay, talc, chalk, quartz, attapulgite, montmorillonite, diatomaceous earth, and the like), pulverized synthetic minerals (for example, highly dispersed silicic acid, alumina, silicates and the like), and the like.

Examples of the solid carrier for granules include pulverized and screened rocks (for example, calcite, marble, pumice, sepiolite, dolomite and the like), synthetic granules of inorganic and organic powder, fine particles of organic materials (for example, sawdust, coconut shells, maize cobs, tobacco stalk and the like), and the like.

Examples of the emulsifier and/or foaming agent include nonionic and anionic emulsifiers [for example, polyoxyethylene fatty acid esters, polyoxyethylene fatty acid alcohol ethers (for example, alkylaryl polyglycol ether), alkylsulfonates, alkylsulfates, arylsulfonates and the like], albumin hydrolyzate, and the like.

Examples of the dispersant include lignin sulfite waste liquor and methylcellulose.

Fixing agents can also be used in the formulations (powders, granules, emulsions), and examples of the fixing agent include carboxymethylcellulose, natural and synthetic polymers (for example, gum arabic, polyvinyl alcohol, polyvinyl acetate, and the like) and the like.

Colorants can also be used, and examples of the colorants include inorganic pigments (for example, iron oxide, titanium oxide, Prussian Blue and the like), organic dyes such as alizarin dyes, azo dyes or metal phthalocyanine dyes, and in addition, trace elements such as the salts of iron, manganese, boron, copper, cobalt, molybdenum and zinc.

The formulations in general can contain the active ingredient in an amount ranging from 0.1 to 95% by weight, and preferably 0.5 to 90% by weight.

The active compound of formula (I) of the present invention can also exist as an admixture with other active compounds, for example, insecticides, poisonous baits, bactericides, miticides, nematicides, fungicides, growth regulators, herbicides and the like, in the form of their commercially useful formulation forms and in the application forms prepared from those formulations. Here, examples of the insecticide mentioned herein include organophosphorus agents, carbamate agents, carboxylate-based drugs, chlorinated hydrocarbon-based chemicals, insecticidal substances produced by microorganisms, and the like.

Furthermore, the active compound of the formula (I) can exist as an admixture with a synergistic agent, and such formulation and application forms can be those commercially useful. The synergistic agent does not have to be necessarily active per se, and is a compound which enhances the action of the active compound.

The content of the active compound of formula (I) of the present invention in a commercially useful application form can be varied within a wide range.

The concentration of the active compound of formula (I) of the present invention in actual usage can be, for example, in the range of 0.0000001 to 100% by weight, and preferably 0.00001 to 1% by weight.

The compound of formula (I) of the present invention can be used through conventional methods that are appropriate for the usage form.

The active compound of the present invention have, when used against hygiene pests and pests associated with stored products, stability effective against alkali on lime materials, and also shows excellent residual effectiveness on wood and soil.

Next, the present invention will be described in more detail by way of Examples, but the present invention is not intended to be limited thereto.

### SYNTHESIS EXAMPLE 1-1

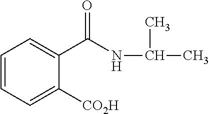

Phthalic anhydride (59.25 g) was dissolved in acetonitrile (1 L), and after isopropyl amine (54.3 g) was added thereto under ice cooling, the mixture was stirred at room temperature for 5 hours. The precipitate was collected by filtration, dissolved in 10% sodium hydroxide aqueous solution, washed with diethyl ether, and adjusted to pH 2 with concentrated hydrochloric acid. The resulting precipitate was filtered, washed with water and air-dried to obtain [2-(1-methylethyl)carbamoyl]benzoic acid (72.0 g).

¹H-NMR (CDCl₃, δ ppm): 1.25 (611, d), 3.45 (1H, q), 7.36-7.47 (3H, m), 7.94-8.02 (2H, m), 10.28-10.70 (1H, m).

### SYNTHESIS EXAMPLE 1-2

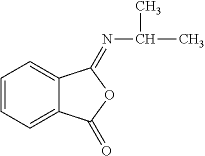

To [2-(1-methylethyl)carbamoyl]benzoic acid (6.27 g), sodium hydrogen carbonate (8.82 g), water (45 mL) and ethyl acetate (90 mL), chlorocarbonic acid methyl (7.09 g) was added, and the mixture was heated with stirring at 50° C. for 20 minutes. After cooled to room temperature, the organic phase was separated, washed with saturated aqueous solution of sodium hydrogen carbonate, and dried over anhydrous sodium sulfate, and the solvent was distilled off to obtain 3-(isopropylimino)-2-benzofuran-1(3H)-one (3.51 g).

¹H-NMR (CDCl₃, δ ppm): 1.15 (6H, d), 4.48-4.59 (1H, m), 7.66-7.85 (4H, m).

### SYNTHESIS EXAMPLE 1-3

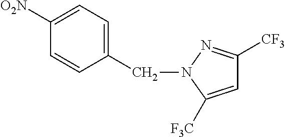

Potassium carbonate (0.66 g) was added to a DMF solution (20 mL) of 4-nitrobenzyl chloride (0.69 g) and 3,5-bis(trifluoromethyl)-1H-pyrazole (0.82 g), and the mixture was stirred at 80° C. for 1 hour. The reaction mixture was poured into water, and the precipitated crude crystal was collected by filtration, washed with water, and air-dried to obtain 1-(4-nitrobenzyl)-3,5-bis(trifluoromethyl)-1H-pyrazole (1.01 g).

¹H-NMR (CDCl₃, δ ppm): 5.57 (2H, s), 6.97 (1H, s), 7.40 (2H, d), 8.23 (2H, d).

### SYNTHESIS EXAMPLE 1-4

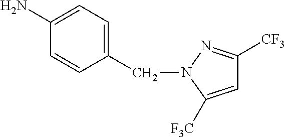

Under ice cooling, to a mixture of tin (II) chloride dihydrate (32.6 g), concentrated hydrochloric acid (33.7 mL) and ethanol (50 mL), 1-(4-nitrobenzyl)-3,5-bis(trifluoromethyl)-1H-pyrazole (9.80 g) was added, and the mixture was stirred for 10 minutes, and then heated and stirred at 70° C. for 1 hour or more. After cooled to room temperature, the reaction mixture was poured into ice, adjusted to pH 11 or above with sodium hydroxide and extracted with t-butylmethylether. The organic phase was washed with water and dried over anhydrous sodium sulfate, and the solvent was distilled off to obtain 4-{[3,5-bis(trifluoromethyl)-1H-pyrazol-1-yl]methyl}aniline (7.57 g).

¹H-NMR (CDCl₃, δ ppm): 3.57-3.89 (2H, m), 5.34 (2H, s), 6.62 (2H, d), 6.86 (1H, s), 7.10 (2H, d).

### SYNTHESIS EXAMPLE 1-5

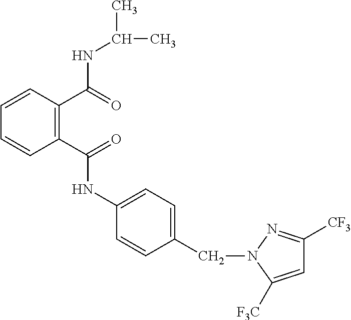

3-(Isopropylimino)-2-benzofuran-1(3H)-one (0.19 g) and 4-{[3,5-bis(trifluoromethyl)-1H-pyrazol-1-yl]methyl}aniline (0.28 g) were dissolved in acetonitrile (5 mL), and p-toluene sulfonic acid-hydrate (0.01 g) was added thereto. The mixture was stirred at room temperature for 3 hours. After the reaction was completed, the solvent was distilled off, and the crude product was purified with silica gel column chromatography (mixed solvent of n-hexane and ethyl acetate) to obtain the objective compound N-(4-{[3,5-bis(trifluoromethyl))-1H-pyrazole-1-yl]methyl}phenyl]-N′-[(1-methylethyl)]-1,2-benzenedicarboxamide (compound No. 8-1)(0.07 g).

Melting point: 176-178° C.

¹H-NMR (CDCl₃, δ ppm): 1.16 (6H, d), 4.12-4.27 (1H, m), 5.44 (2H, s), 6.05 (1H, d), 6.91 (1H, s), 7.19-7.57 (5H, m), 7.67 (2H, d), 7.84 (1H, d), 9.42 (1H, bs).

### SYNTHESIS EXAMPLE 2-1

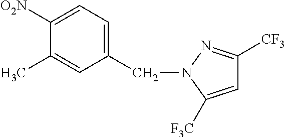

3-Methyl-4-nitrobenzyl chloride (1.81 g), 3,5-bis(trifluoromethyl)-1H-pyrazole (2.0 g) and potassium carbonate (1.63 g) were stirred at 60° C. in DMF (20 mL) for 1 hour. After the reaction was completed, water (100 mL) was added, and the mixture was extracted with ethyl acetate. The organic phase was washed with saturated brine (100 mL) and dried with anhydrous sodium sulfate. After the solvent was distilled off, the resulting crude product was purified with silica gel column chromatography to obtain the objective compound 1-(3-methyl-4-nitrobenzyl)-3,5-bis(trifluoromethyl)-1H-pyrazole (3.30 g).

¹H-NMR (CDCl₃, δ ppm): 2.59 (3H, s), 5.50 (2H, s), 6.90 (1H, s), 7.1-7.2 (2H, m), 8.00 (1H, d).

### SYNTHESIS EXAMPLE 2-2

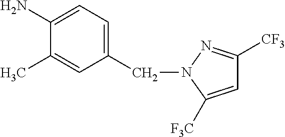

1-(3-methyl-4-nitrobenzyl)-3,5-bis(trifluoromethyl)-1H-pyrazole (17.66 g) and iron powder (13.69 g) were heated and stirred in acetic acid (150 mL) at 40° C. for 5 hours. After the reaction was completed, and insoluble material was filtered off with Celite, the filtrate was concentrated under reduced pressure. 1 N sodium hydroxide aqueous solution (200 mL) and ethyl acetate (200 mL) were added to the crude product, the organic phase was separated, washed with water and dried over anhydrous magnesium sulfate, and then the solvent was distilled off to obtain the objective compound 4-{[3,5-bis(trifluoromethyl)-1H-pyrazol-1-yl]methyl}-2-methylaniline (13.0 g).

¹H-NMR (CDCl₃, δ ppm): 2.14 (3H, s), 3.66 (2H, m), 5.32 (2H, s), 6.62 (1H, d), 6.89 (1H, s), 6.8-7.1 (2H, m).

### SYNTHESIS EXAMPLE 2-3

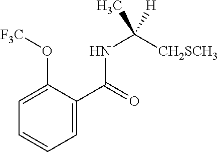

2-(Trifluoro methoxy)benzoyl chloride (4.49 g) was added to a THF solution (40 mL) of (2S)-1-(methylthio)propane-2-amine (2.31 g) and triethylamine (3.35 mL) at 5° C., and the mixture was stirred for one hour at the same temperature. After 2N hydrochloric acid (20 mL) and ethyl acetate (60 mL) were added to the reaction mixture, the organic phase was separated, washed with 2N hydrochloric acid and saturated aqueous solution of sodium hydrogen carbonate, and dried over anhydrous sodium sulfate, and the solvent was distilled off to obtain N-[(1S)-1-methyl-2-(methylthio)ethyl]-2-(trifluoro methoxy)bezamide (4.18 g).

¹H-NMR (CDCl₃, δ ppm): 1.35 (3H, d), 2.18 (3H, s), 2.67 (1H, dd), 2.79 (1H, dd), 4.36-4.49 (1H, m), 6.54-6.67 (1H, m), 7.36-7.54 (2H, m), 7.98 (1H, d).

### SYNTHESIS EXAMPLE 2-4

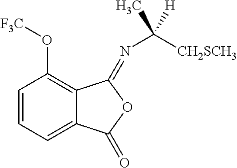

To a THF solution of N-[(1S)-1-methyl-2-(methylthio)ethyl]-2-(trifluoro methoxy)benzamide (0.88 g) and N,N,N′,N′-tetra methyl ethylenediamine (0.77 g), 15% n-butyl lithium hexane solution (4 mL) was added dropwise at −70° C., and subsequently the mixture was stirred at −70° C. for 1 hour. After carbon dioxide (1.3 g) was blown into the reaction mixture with further stirring at −70° C. for 3 hours, the reaction mixture was acidified with 2N hydrochloric acid and extracted with ethyl acetate. The organic phase was washed with 2N hydrochloric acid and saturated brine, and dried over anhydrous sodium sulfate. The solvent was distilled off to obtain the crude 3-(trifluoromethoxy)-2-{[(1S)-1-methyl-2-(methylthio)ethyl]carbamoyl}benzoic acid (0.9 g). This crude product was dissolved in ethyl acetate (20 mL), and sodium hydrogen carbonate (0.45 g), water (10 mL) and methyl chlorocarbonate (0.50 g) were added thereto, and the mixture was heated and stirred at 50° C. for 20 minutes. After cooled to room temperature, the organic phase was separated, washed with saturated aqueous solution of sodium hydrogen carbonate and dried over anhydrous sodium sulfate, and the solvent was distilled off to obtain 3-{[(1S)-1-methyl-2-(methylthio)ethyl]imino}-4-(trifluoromethoxy)-2-benzofuran-1(3H)-one (0.10 g), which was subjected to the next reaction without purification.

### SYNTHESIS EXAMPLE 2-5

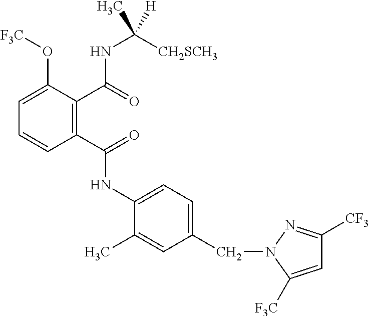

3-{[(1S)-1-Methyl-2-(methylthio)ethyl]imino}-4-(trifluoromethoxy)-2-benzofuran-1(3H)-one (0.10 g) and 4-{[3,5-bis(trifluoromethyl)-1H-pyrazole-1-yl]methyl}aniline (0.10 g) were dissolved in acetonitrile (1.6 mL), and p-toluene sulfonic acid monohydrate (0.003 g) was added thereto. Then, the mixture was stirred at room temperature for 3 hours. After the reaction was completed, the solvent was distilled off, and the crude product was purified with silica gel column chromatography (mixed solvent of n-hexane and ethyl acetate) to obtain the objective compound N¹-(4-{[3,5-bis(trifluoromethyl)-1H-pyrazol-1-yl]methyl}-2-methylphenyl)-N²-[(1S)-1-methyl-2-(methylthio)ethyl]-3-(trifluoromethoxy)-1,2-benzenedicarboxamide (compound No. 13-13)(0.15 g).

Melting point: 158-160° C.

¹H-NMR (CDCl₃, δ ppm): 1.24 (3H, d), 1.93 (3H, s), 2.31 (3H, s), 2.53 (1H, dd), 2.61 (1H, dd), 4.29-4.41 (1H, m), 5.41 (2H, s), 6.11 (1H, d), 6.91 (1H, s), 7.11 (1H, s), 7.12 (1H, d), 7.46 (1H, d), 7.57 (1H, dd), 7.81 (1H, d), 8.07 (1H, d), 8.44 (1H, bs).

### SYNTHESIS EXAMPLE 3-1

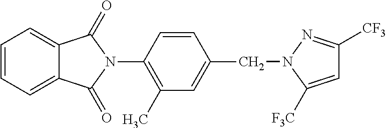

An acetic acid solution (17 mL) of phthalic anhydride (1.78 g) and 4-{[3,5-bis(trifluoromethyl)-1H-pyrazol-1-yl]methyl}-2-methylaniline (3.88 g) was heated to reflux for 3 hours. After the reaction was completed, acetic acid was distilled off under reduced pressure, and the resulting crude crystal was washed with t-butylmethylether/petroleum ether mixed solvent to obtain 2-(4-{[3,5-bis(trifluoromethyl)-1H-pyrazol-1-yl]methyl}-2-methylphenyl)-1H-isoindole-1,3(2H)-dione (4.70 g).

Melting point: 182-183° C.

### SYNTHESIS EXAMPLE 3-2

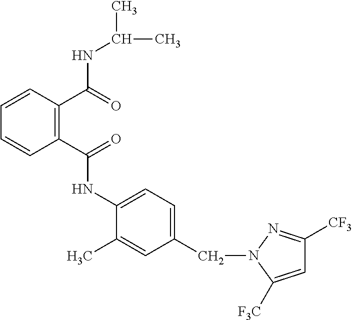

2-(4-{[3,5-bis(trifluoromethyl)-1H-pyrazol-1-yl]methyl-2-methylphenyl)-1H-isoindole-1,3(2H)-dione (0.45 g) and isopropylamine (0.18 g) were dissolved in dioxane (8 mL), and acetic acid (0.01 g) was added thereto. The mixture was heated to reflux for 3 hours. After the reaction was completed, the solvent was distilled off, and the crude product was purified with silica gel column chromatography (mixed solvent of n-hexane and ethyl acetate) to obtain N-(4-{[3,5-bis(trifluoromethyl)-1H-pyrazol-1-yl]methyl}-2-methylphenyl)-N′-[(1-methylethyl)]-1,2-benzenedicarboxamide (0.35 g) (compound No. 13-1).

Melting point: 170-172° C.

¹H-NMR (CDCl₃, δ ppm): 1.17 (6H, d), 2.31 (3H, s), 4.13-4.26 (1H, m), 5.41 (2H, s), 6.04 (1H, d), 6.90 (1H, s), 7.11 (1H, s), 7.14 (1H, d), 7.48-7.57 (3H, m), 7.81-7.87 (1H, m), 8.04 (1H, d), 8.70 (1H, bs).

### SYNTHESIS EXAMPLE 4-1

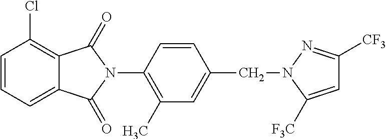

As similar to SYNTHESIS EXAMPLE 3-1, 2-(4-{[3,5-bis(trifluoromethyl)-1H-pyrazol-1-yl]methyl}-2-methylphenyl)-4-chloro-1H-isoindole-1,3(2H)-dione (13.61 g) was obtained from 3-chlorophthalic anhydride (5.44 g) and 4-{[3,5-bis(trifluoromethyl)-1H-pyrazol-1-yl]methyl}-2-methylaniline (9.70 g).

¹H-NMR (CDCl₃, δ ppm): 2.20 (3H, s), 5.48 (2H, s), 6.93 (1H, s), 7.16-7.32 (3H, m), 7.70-7.92 (3H, m).

### SYNTHESIS EXAMPLE 4-2

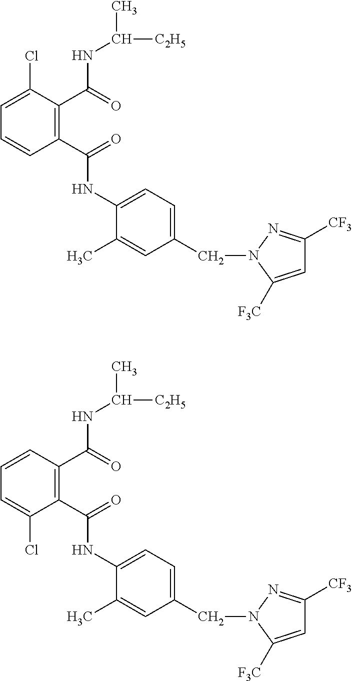

2-(4-{[3,5-bis(trifluoromethyl)-1H-pyrazol-1-yl]methyl}-2-methylphenyl)-4-chloro-1H-isoindole-1,3(2H)— dione (0.63 g) and sec-butylamine (0.28 g) were dissolved in dioxane (10 mL), and acetic acid (0.01 g) was added thereto. The mixture was heated to reflux for 3 hours. After the reaction was completed, the solvent was distilled off, and the crude product was purified with silica gel column chromatography (mixed solvent of n-hexane and ethyl acetate) to obtain, as the first elution portion, N¹-(4-{[3,5-bis(trifluoromethyl)-1H-pyrazol-1-yl]methyl}-2-methylphenyl)-3-chloro-N²-(butan-2-yl)-1,2-benzenedicarboxamide (0.36 g) as colorless crystal; and as the second elution portion, N²-(4-{[3,5-bis(trifluoromethyl)-1H-pyrazol-1-yl]methyl}-2-methylphenyl)-3-chloro-N¹-(butan-2-yl)-1,2-benzenedicarboxamide (0.27 g) as colorless crystal. N¹-(4-{[3,5-bis(trifluoromethyl)-1H-pyrazol-1-yl]methyl}-2-methylphenyl)-3-chloro-N²-(butan-2-yl)-1,2-benzenedicarboxamide (compound No. 13-20)

Melting point: 187-188° C.

¹H-NMR (CDCl₃, δ ppm): 0.87 (3H, t), 1.10 (3H, d), 1.40-1.52 (2H, m), 2.30 (3H, s), 3.97-4.12 (1H, m), 5.41 (2H, s), 5.88 (1H, d), 6.91 (1H, s), 7.07-7.13 (2H, m), 7.43 (1H, dd), 7.54 (1H, d), 7.72 (1H, d), 8.06 (1H, d), 8.43 (1H, bs)
N²-(4-{[3,5-bis(trifluoromethyl)-1H-pyrazol-1-yl]methyl}-2-methylphenyl)-3-chloro-N¹-(butan-2-yl)-1,2-benzenedicarboxamide (compound No. 13-22)
   Melting point: 211-212° C.

¹H-NMR (CDCl₃, δ ppm): 0.83 (3H, t), 1.08 (3H, d), 1.39-1.50 (2H, m), 2.29 (3H, s), 3.90-4.03 (1H, m), 5.42 (2H, s), 6.22 (1H, d), 6.91 (1H, s), 7.10-7.17 (2H, m), 7.48-7.58 (3H, m), 7.98 (1H, d).

### SYNTHESIS EXAMPLE 5-1

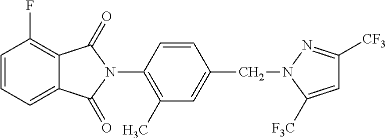

As similar to SYNTHESIS EXAMPLE 3-1, 2-(4-{[3,5-bis(trifluoromethyl)-1H-pyrazol-1-yl]methyl}-2-methylphenyl)-4-fluoro-1H-isoindole-1,3(2H)-dione (10.80 g) was obtained from 3-fluorophthalic anhydride (4.93 g) and 4-{[3,5-bis(trifluoromethyl)-1H-pyrazol-1-yl]methyl}-2-methylaniline (9.70 g).

¹H-NMR (CDCl₃, δ ppm): 2.21 (3H, s), 5.47 (2H, s), 6.93 (1H, s), 7.16-7.50 (4H, m), 7.75-7.85 (2H, m).

### SYNTHESIS EXAMPLE 5-2

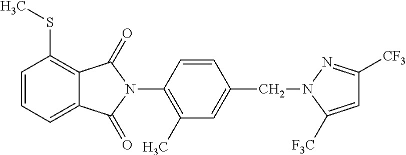

To a DMF solution (24 mL) of 2-(4-{[3,5-bis(trifluoromethyl)-1H-pyrazol-1-yl]methyl}-2-methylphenyl)-4-fluoro-1H-isoindole-1,3(2H)-dione (2.83 g), 15% aqueous solution of sodium thiomethoxide (2.94 g) was added, and the mixture was stirred for 3 hours. The reaction mixture was poured into water, and the precipitated crude crystal was collected by filtration, washed with water and air-dried to obtain 2-(4-{[3,5-bis(trifluoromethyl)-1H-pyrazol-1-yl]methyl}-2-methylphenyl)-4-methylthio-1H-isoindole-1,3(2H)-dione (2.36 g).

Melting point: 163-164° C.

¹H-NMR (CDCl₃, δ ppm): 2.19 (3H, s), 2.59 (3H, s), 5.48 (2H, s), 6.93 (1H, s), 7.13-7.79 (6H, m).

### SYNTHESIS EXAMPLE 5-3

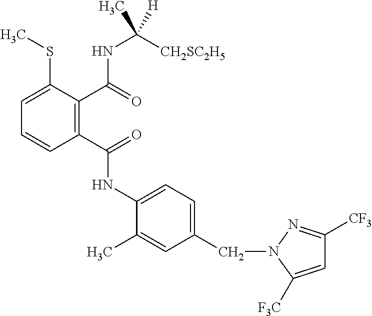

As similar to SYNTHESIS EXAMPLE 4-2, N¹-(4-{[3,5-bis(trifluoromethyl)-1H-pyrazol-1-yl]methyl}-2-methylphenyl)-N²-[(1S)-1-methyl-2-(ethylthio)ethyl]-3-methylthio-1,2-benzenedicarboxamide (0.05 g) (compound No. 13-291) was obtained from 2-(4-{[3,5-bis(trifluoromethyl)-1H-pyrazol-1-yl]methyl}-2-methylphenyl)-4-methylthio-1H-isoindole-1,3(2H)-dione (0.50 g) and (2S)-1-(ethylthio)propan-2-amine (0.36 g).

¹H-NMR (CDCl₃, δ ppm): 1.10 (3H, t), 1.20 (3H, d), 2.28 (3H, s), 2.39 (2H, q), 2.50 (3H, s), 2.52 (1H, dd), 2.66 (1H, dd), 4.21-4.36 (1H, m), 5.40 (2H, s), 6.41 (1H, d), 6.91 (1H, s), 7.06-7.19 (2H, m), 7.36-7.56 (3H, m), 8.06 (1H, d), 8.34 (1H, bs).

### SYNTHESIS EXAMPLE 6-1

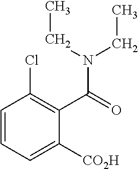

Diethyl amine (4.39 g) was added to a dimethylacetamide solution (116 mL) of 3-chlorophthalic acid anhydride (9.13 g) at −5° C., and the mixture was stirred at 0° C. for 1 hour. Then, 30% sodium hydroxide aqueous solution (6.5 g) was added thereto, and the mixture was stirred at 0° C. for another 1 hour. After t-butylmethylether (200 mL) was added to the reaction liquor solution and stirred for 10 minutes, the precipitated crystal was collected by filtration, washed with t-butylmethylether and air-dried. The obtained crystal was dissolved in water (50 mL), and the solution was adjusted to pH 4 bp addition of 2N hydrochloric acid at 5° C. The precipitated crystal was collected by filtration, washed with water and dried under reduced pressure to obtain 3-chloro-2-(diethylcarbamoyl)-benzoic acid (9.24 g).

¹H-NMR (CDCl₃, δ ppm): 1.09 (3H, t), 1.28 (3H, t), 3.24 (2H, q), 3.61 (2H, q), 7.37 (1H, t), 7.48-7.58 (1H, m), 8.00 (1H, d).

### SYNTHESIS EXAMPLE 6-2

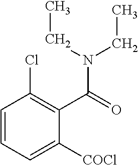

Oxalyl chloride (1.14 g) was added to a 1,2-dichloroethane solution (60 mL) of 3-chloro-2-(diethylcarbamoyl)benzoic acid (1.39 g) and DMF (0.01 g), and the mixture was heated and stirred at 60° C. until gas generation ceased. After the reaction was completed, the solvent was distilled off to obtain 3-chloro-2-(diethylcarbamoyl)benzoylchloride (1.25 g) as colorless oily matter, which was subjected to the next reaction without purification.

### SYNTHESIS EXAMPLE 6-3

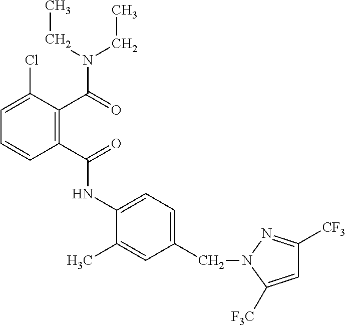

4-{[3,5-bis(trifluoromethyl)-1H-pyrazol-1-yl]methyl}-2-methylaniline (0.59 g) and triethylamine (0.22 g) were dissolved in THF (7 mL), and after 3-chloro-2-(diethylcarbamoyl)benzoylchloride (0.50 g) was added under ice cooling, the mixture was stirred at room temperature for 3 hours. After the reaction was completed, the reaction mixture was diluted with ethyl acetate, washed with 2N hydrochloric acid and saturated aqueous solution of sodium hydrogen carbonate, and dried over anhydrous sodium sulfate. The solvent was distilled off, and the crude product was purified with silica gel column chromatography (mixed solvent of n-hexane and ethyl acetate) to obtain N¹-(4-{[3,5-bis(trifluoromethyl)-1H-pyrazole-1-yl]methyl}-2-methylphenyl)-3-chloro-N²,N²-diethyl-1,2-benzenedicarboxamide (0.50 g) (compound No. 1-1).

Melting point: 105-107° C.

### SYNTHESIS EXAMPLE 7-1

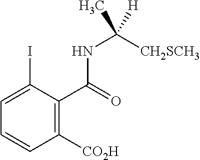

To a solution of 3-iodophthalic anhydride (32.5 g) in dimethylformamide (300 mL), a solution of (2S)-1-(methylthio)propan-2-amine (15.0 g) in dimethylformamide (50 mL) was added dropwise at −10° C. for 3 hours, and the mixture was stirred at −10° C. for additional 3 hours. After addition of 40% sodium hydroxide aqueous solution (15 g), the solvent was distilled off under reduced pressure, and the crude product was dissolved in water (500 mL) and washed with diisopropyl ether. The water phase was separated, adjusted to pH 1 with concentrated hydrochloric acid and extracted with diisopropyl ether. The organic phase was washed with saturated brine and dried over anhydrous sodium sulfate. The solvent was distilled off, and the resulting crude crystal was washed with a small amount of diisopropyl ether and air-dried to obtain 3-iodo-2-{[(1S)-1-methyl-2-(methylthio)ethyl]-carbamoyl}benzoic acid (32.2 g).

Melting point: 132-134° C.

### SYNTHESIS EXAMPLE 7-2

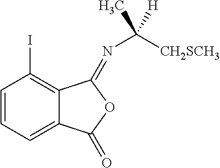

As similar to SYNTHESIS EXAMPLE 1-2, 4-iodo-3-{[(1S)-1-methyl-2-(methylthio)ethyl]imino}-2-benzofuran-1(3H)-one (3.3 g) was obtained from 3-iodo-2-{[(1S)-1-methyl-2-(methylthio)ethyl]carbamoyl}benzoic acid (5.7 g).

¹H-NMR (CDCl₃, δ ppm): 1.39 (3H, t), 2.19 (3H, s), 2.74-2.80 (2H, m), 4.31-4.43 (1H, m), 7.36 (1H, t), 7.93 (1H, d), 8.25 (1H, d).

### SYNTHESIS EXAMPLE 7-3

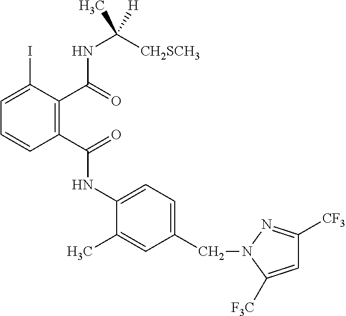

As similar to SYNTHESIS EXAMPLE 1-5, N¹-(4-{[3,5-bis(trifluoromethyl)-1H-pyrazol-1-yl]methyl}-2-methylphenyl)-3-iodo-N²-[(1S)-1-methyl-2-(methylthio)ethyl]-1,2-benzenedicarboxamide (5.0 g) was obtained from 4-iodo-3-{[(1S)-1-methyl-2-(methylthio)ethyl]imino}-2-benzofuran-1(3H)-one (3.61 g) and 4-{[3,5-bis(trifluoromethyl)-1H-pyrazol-1-yl]methyl}-2-methylaniline (3.23 g).

Melting point: 85-93° C.

### SYNTHESIS EXAMPLE 7-4

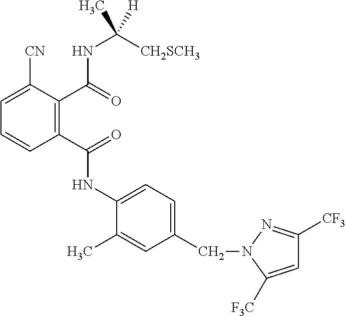

A dioxane solution (3 mL) of N′-(4-{[3,5-bis(trifluoromethyl)-1H-pyrazol-1-yl]methyl}-2-methylphenyl)-3-iodo-N²-[(1S)-1-methyl-2-(methylthio)ethyl]-1,2-benzenedicarboxamide (0.41 g), cuprous cyanide (0.13 g), tris(dibenzylideneacetone)-dipalladium chloroform complex (0.03 g) and 1,1′-bis(diphenylphosphine)ferrocene (0.05 g) was heated and stirred at 80 to 90° C. for 5 hours. After cooled to room temperature, insoluble material was filtered off with Celite, and the filtrate was concentrated under reduced pressure. The crude product was subjected to silica gel column chromatography (mixed solvent of n-hexane and ethyl acetate) to obtain the objective compound N¹-(4-{[3,5-bis(trifluoromethyl)-1H-pyrazol-1-yl]methyl}-2-methylphenyl)-3-cyano-N²-[(1S)-1-methyl-2-(methylthio)ethyl]-1,2-benzenedicarboxamide (0.41 g) (compound No. 13-172).

¹H-NMR (CDCl₃, δ ppm): 1.63 (3H, d), 2.11 (3H, s), 2.40 (3H, s), 2.82-2.94 (2H, m), 4.75-4.94 (1H, m), 5.43 (2H, s), 6.91 (1H, s), 7.10-7.21 (2H, m), 7.28-7.39 (1H, m), 7.55-7.73 (1H, m), 7.79-7.94 (2H, m), 8.76 (1H, d), 8.89-9.00 (1H, m).

### SYNTHESIS EXAMPLE 8-1

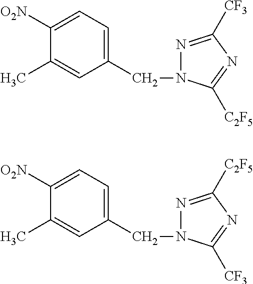

3-Methyl-4-nitrobenzyl chloride (1.64 g), 5-pentafluoroethyl-3-trifluoromethyl-1H-triazole (2.25 g), potassium carbonate (1.83 g), 18-crown-6 (0.12 g) and tetrabutyl ammonium iodide (0.16 g) were heated to reflux in propionitrile (22 mL) for 2 hours. After cooled to room temperature, water (100 mL) was added to the reaction mixture, and extracted with ethyl acetate (100 mL). The organic phase was washed sequentially with 5% sodium hydroxide aqueous solution, 0.5N hydrochloric acid and saturated brine, and dried over anhydrous sodium sulfate. The solvent was distilled off under reduced pressure, the resulting crude product was purified with silica gel column chromatography (mixed solvent of n-hexane and ethyl acetate) to obtain, as the first elution portion, 1-(3-methyl-4-nitrobenzyl)-5-pentafluoroethyl-3-trifluoromethyl-1H-[1,2,4]-triazole (0.46 g);

¹H-NMR (CDCl₃, δ ppm): 2.62 (3H, s), 5.59 (2H, s), 7.26-7.31 (2H, m), 7.99 (1H, d).

¹⁹F-NMR (CDCl₃, δ ppm): −65.76, −83.29, −115.85;

and, as the second elution portion, 1-(3-methyl-4-nitrobenzyl)-3-pentafluoroethyl-5-trifluoromethyl-1H-[1,2,4]-triazole (0.35 g).

¹H-NMR (CDCl₃, δ ppm): 2.71 (3H, s), 5.59 (2H, s), 7.23-7.31 (2H, m), 7.99 (1H, d).

¹⁹F-NMR (CDCl₃, δ ppm): −62.25, −84.27, −115.85.

### SYNTHESIS EXAMPLE 8-2

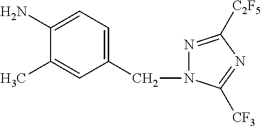

Tin chloride dihydrate (2.26 g) and concentrated hydrochloric acid (2.3 mL) were added to an ethanol solution (3 mL) of 1-(3-methyl-4-nitrobenzyl)-3-pentafluoroethyl-5-trifluoromethyl-1H-1,2,4-triazole (0.81 g) under ice cooling. After the reaction mixture was heated and stirred at 70° C., poured into iced water, made alikaline with sodium hydroxide and extracted with t-butylmethylether, the organic phase was washed with water and dried with anhydrous sodium sulfate. The solvent was distilled under reduced pressure to obtain the objective compound 2-methyl-4-{[3-(pentafluoroethyl)-5-(trifluoromethyl)-1H-1,2,4-triazole-1-yl]methyl}aniline (0.64 g).

Melting point: 60-63° C.

### SYNTHESIS EXAMPLE 8-3

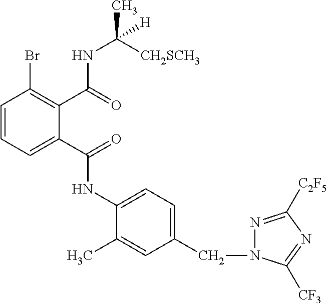

As similar to SYNTHESIS EXAMPLE 1-5, 3-bromo-N²-[(1S)-1-methyl-2-(methylthio)ethyl]-N¹-(2-methyl-4-{[3-(pentafluoroethyl)-5-(trifluoromethyl-1H-1,2,4-triazol-1-yl]methyl}phenyl)-1,2-benzenedicarboxamide (0.63 g) was obtained from 4-bromo-3-{[(1S)-1-methyl-2-(methylthio)ethyl]imino}-2-benzofuran-1(3H)-one (0.61 g) and 2-methyl-4-{[3-(pentafluoroethyl)-5-(trifluoromethyl)-1H-1,2,4-triazol-1-yl]methyl}aniline (0.60 g).

Melting point: 80-85° C.

### SYNTHESIS EXAMPLE 8-4

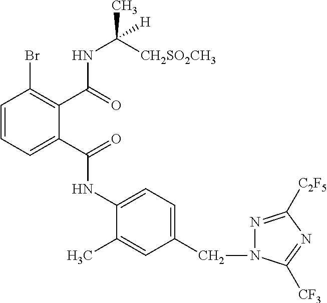

3-Bromo-N²-[(1S)-1-methyl-2-(methylthio)ethyl]-N¹-(2-methyl-4-{[3-(pentafluoroethyl)-5-(trifluoromethyl)-1H-1,2,4-triazol-1-yl]methyl}phenyl)-1,2-benzenedicarboxamide (0.73 g) was dissolved in dichloromethane, and after m-chloroperbenzoic acid (0.66 g) was added under ice cooling, the mixture was stirred at room temperature for 2 hours. After the reaction was completed, the reaction mixture was washed sequentially with a sodium thiosulfate aqueous solution, saturated sodium bicarbonate water and saturated brine, and dried over anhydrous sodium sulfate. The solvent was distilled off, and the resulting crude crystal was purified with petroleum ether to obtain 3-bromo-N²-[(1S)-1-methyl-2-(methylsulfonyl)ethyl]-N¹-(2-methyl-4-{[3-(pentafluoroethyl)-5-(trifluoromethyl)-1H-1,2,4-triazol-1-yl]methyl}phenyl)-1,2-benzenedicarboxamide (0.42 g).

Melting point: 104-108° C.

### SYNTHESIS EXAMPLE 8-5

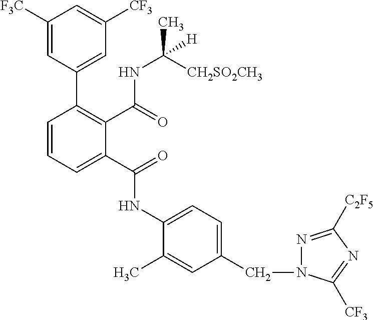

A mixture of 3-bromo-N²-[(1S)-1-methyl-2-(methylsulfonyl)ethyl]-N¹-(2-methyl-4-{[3-(pentafluoroethyl)-5-(trifluoromethyl)-1H-1,2,4-triazol-1-yl]methyl}phenyl)-1,2-benzenedicarboxamide (0.144 g), 3,5-bis(trifluoromethyl)phenylboric acid (0.06 g), tetra-n-butyl ammonium bromide (0.06 g), potassium carbonate (0.07 g), acetic acid palladium (0.001 g) and water (3 mL) was heated and stirred at 70° C. for 1 hour. After cooled to room temperature, the reaction mixture was extracted with ethyl acetate and dried over anhydrous sodium sulfate. The solvent was distilled off, and the resulting crude product was subjected to silica gel column chromatography (mixed solvent of n-hexane and ethyl acetate) to obtain the objective compound N²-[(1S-methyl-2-(methylsulfonyl)ethyl]-N³-(2-methyl-4-{[3-(pentafluoroethyl)-5-(trifluoromethyl)-1H-1,2,4-triazol-1-yl]methyl}phenyl)-3′,5′-bis(trifluoromethyl)biphenyl-2,3-dicarboxamide (0.04 g) was obtained (compound No. 14-264).

Melting point: 201° C.

### SYNTHESIS EXAMPLE 9-1

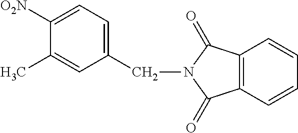

3-methyl-4-nitrobenzyl chloride was added to a DMF suspension (100 mL) of potassium phthalimide (18.5 g), and the mixture was stirred at room temperature for 5 hours. The reaction mixture was poured into iced water, and the precipitated crystal was collected by filtration, washed with water and air-dried to obtain 2-(3-methyl-4-nitrobenzyl)-1H-isoindole-1,3(2H)-dione (25.8 g).

Melting point: 139 to 141° C.

### SYNTHESIS EXAMPLE 9-2

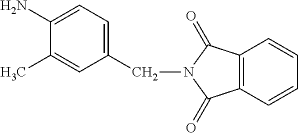

Tin chloride dihydrate (33.86 g) and concentrated hydrochloric acid (35 mL) were added to an ethanol solution (50 mL) of 2-(3-methyl-4-nitrobenzyl)-1H-isoindole-1,3(2H)-dione (9.36 g) under ice cooling. After heated and stirred at 70° C., the reaction mixture was poured into iced water, made alkaline with sodium hydroxide and extracted with t-butylmethylether. The organic phase was washed with water and dried over anhydrous sodium sulfate. The solvent was distilled off under reduced pressure to obtain the objective compound 2-(4-amino-3-methylbenzyl)-1H-isoindole-1,3(2H)-dione (7.70 g).

¹H-NMR (CDCl₃, δ ppm): 2.14 (3H, s), 4.71 (2H, s), 6.60 (1H, d), 7.11-7.19 (2H, m), 7.65-7.87 (4H, m).

### SYNTHESIS EXAMPLE 9-3

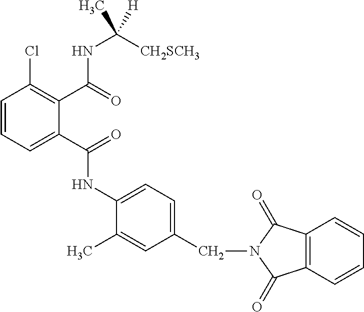

As similar to SYNTHESIS EXAMPLE 1-5, 3-chloro-N¹-{4-[(1,3-dioxo-1,3-dihydro-2H-isoindol-2-yl)methyl]-2-methylphenyl]-N²-[(1S)-1-methyl-2-(methylthio)ethyl]-1,2-benzenedicarboxamide (2.45 g) was obtained from 4-chloro-3-{[(1S)-1-methyl-2-(methylthio)ethyl]imino}-2-benzofuran-1(3H)-one (1.52 g) and 2-(4-amino-3-methylbenzyl)-1H-isoindole-1,3(2H)-dione (1.50 g).

Melting point: 166-168° C.

### SYNTHESIS EXAMPLE 9-4

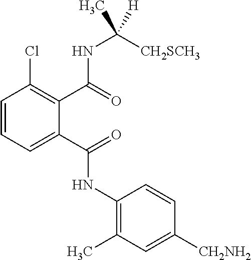

Hydrazine hydrate (1.0 g) was added to an ethanol solution (5 mL) of 3-chloro-N′-{4-[(1,3-dioxo-1,3-dihydro-2H-isoindol-2-yl)methyl]-2-methylphenyl}-N²-[(1S)-1-methyl-2-(methylthio)ethyl]-1,2-benzenedicarboxamide (1.07 g) at room temperature, and the mixture was heated and stirred at 60° C. for 30 minutes. The precipitate was filtered, and washed with ethanol. After the filtrate was concentrated under reduced pressure, the resulting crude product was dissolved in ethyl acetate, and washed sequentially with 5% sodium hydroxide aqueous solution, water and saturated brine, and dried over anhydrous sodium sulfate. The solvent was distilled off to obtain N-[4-(aminomethyl)-2-methylphenyl]-3-chloro-N²-[(1S)-1-methyl-2-(methylthio)ethyl]-1,2-benzenedicarboxamide (0.65 g).

¹H-NMR (CDCl₃, δ ppm): 1.26 (3H, d), 1.47-1.51 (2H, m), 2.00 (3H, s), 2.31 (3H, s), 2.55 (1H, dd), 2.66 (1H, dd), 3.81 (2H, s), 4.29-4.44 (1H, m), 6.29 (1H, d), 7.12-7.19 (2H, m), 7.44 (1H, t), 7.58 (1H, d), 7.75 (1H, d), 7.97 (1H, d), 7.97 (1H, bs).

### SYNTHESIS EXAMPLE 9-5

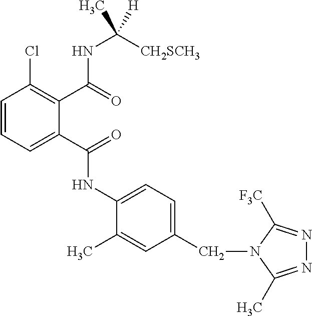

N¹-[4-(aminomethyl)-2-methylphenyl]-3-chloro-N²-[(1S)-1-methyl-2-(methylthio)ethyl]-1,2-benzene-dicarboxamide (122 mg), 3,5-bis(trifluoromethyl)-1,3,4-oxadiazole (124 mg) and methanol (1 mL) were added into a glass vial, which was then tightly sealed, and the mixture was heated and stirred at 90° C. for one hour. After the reaction was completed, the solvent was distilled off, and the crude product was subjected to silica gel chromatography (mixed solvent of n-hexane and ethyl acetate) to obtain 3-chloro-N²-[(1S)-1-methyl-2-(methylthio)ethyl]-N¹-(2-methyl-4-{[3-methyl-5-(trifluoromethyl)-4H-1,2,4-triazol-4-yl]methyl}phenyl)-1,2-benzenedicarboxamide (85 mg) (compound No. 14-146).

Melting point: 168-170° C.

### SYNTHESIS EXAMPLE 10

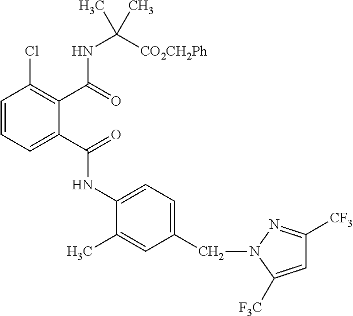

As similar to SYNTHESIS SAMPLE 1-5, benzyl N-({2-[(4-{[3,5-bis(trifluoromethyl)-1H-pyrazol-1-yl]methyl}-2-methylphenyl)carbamoyl]-6-chlorobenzoyl}-2-methyl alaninate (1.50 g) (compound No. 13-120) was obtained from 4-{[3,5-bis(trifluoromethyl)-1H-pyrazol-1-yl]methyl}-2-methylaniline (2.23 g) and benzyl N-(7-chloro-3-oxo-2-benzofuran-1(3H)-ylidene)-2-methylalaninate (2.40 g).

Melting point: 171-173° C.

¹H-NMR (CDCl₃, δ ppm): 1.53 (6H, s), 2.27 (3H, s), 5.05 (2H, s), 5.38 (2H, s), 6.74-8.32 (14H, m).

### SYNTHESIS EXAMPLE 11

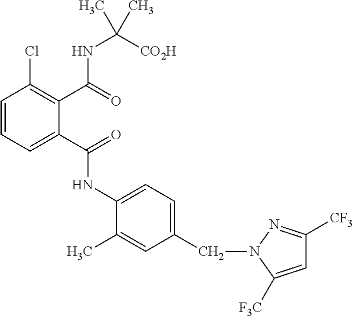

Benzyl N-({2-[(4-{[3,5-bis(trifluoromethyl)-1H-pyrazol-1-yl]methyl}-2-methylphenyl)carbamoyl]-6-chlorobenzoyl}-2-methyl alaninate (1.00 g) was dissolved in dichloromethane (25 mL), and 1 Mol/L solution (6 mL) of boron tribromide in dichloromethane was added under ice cooling. The mixture was stirred at 0° C. for 2 hours, and then at room temperature for additional 2 hours. After the reaction was completed, water (5 mL) was added, and dichloromethane was distilled off. The crude product was poured into saturated brine, and after addition of concentrated hydrochloric acid (2 mL), the extraction wad carried out with ethyl acetate. After the organic phase was dried over anhydrous magnesium sulfate, the solvent was distilled off to obtain N-({2-[(4-{[3,5-bis(trifluoromethyl)-1H-pyrazol-1-yl]methyl}-2-methylphenyl)carbamoyl]-6-chlorobenzoyl}-2-methyl alanine (0.85 g) (compound No. 13-116).

Melting point: 179-184° C.

### SYNTHESIS EXAMPLE 12

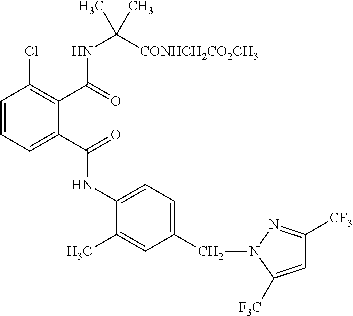

N-({2-[(4-{[3,5-bis(trifluoromethyl)-1H-pyrazole-1-yl]methyl}-2-methylphenyl)carbamoyl]-6-chlorobenzoyl}-2-methylalanine (250 mg), glycine methyl ester hydrochloric acid salt (58 mg), N-hydroxy benzotriazole (63 mg) and triethylamine (51 mg) were dissolved in DMF (5 mL), and at room temperature, 1-(3-dimethylaminopropyl)-3-ethylcarbodiimide hydrochloride (89 mg) was added thereto, and the mixture was stirred for 17 hours. The reaction mixture was poured into cold saturated brine, and extracted with ethyl acetate. After the organic phase was dried over anhydrous magnesium sulfate, the solvent was distilled off. The resulting crude product was subjected to silica gel column chromatography (mixed solvent of n-hexane and ethyl acetate) to obtain methyl N-{2-[(4-{[3,5-bis(trifluoromethyl)-1H-pyrazol-1-yl]methyl}-2-methylphenyl)carbamoyl]-6-chlorobenzoyl)-2-methylalanyl glycinate (220 mg) (compound No. 13-121).

¹H-NMR (CDCl₃, δ ppm): 1.58 (3H, s), 1.62 (6H, s), 2.29 (3H, s), 3.66 (3H, s), 3.75 (2H, d), 5.42 (2H, s), 6.27 (1H, s), 6.93-8.36 (9H, m).

### SYNTHESIS EXAMPLE 13

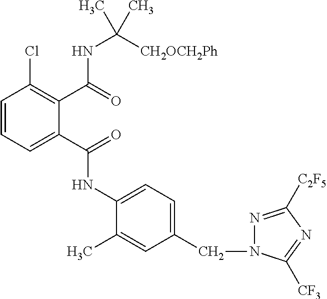

As similar to SYNTHESIS EXAMPLE 1-5, N²-[2-(benzyloxy)-1,1-dimethylethyl]-3-chloro-N¹-(2-methyl-4-{[3-(pentafluoroethyl)-5-(trifluoromethyl)-1H-1,2,4-triazol-1-yl]methyl}phenyl)-1,2-benzenedicarboxamide (1.30 g) was obtained (compound No. 14-133) from 2-methyl-4-{[3-(pentafluoroethyl)-5-(trifluoromethyl)-1H-1,2,4-triazol-1-yl]methyl}aniline (2.15 g) and 3-{[2-(benzyloxy)-1,1-dimethylethyl]imino}-4-chloro-2-benzofuran-1(3H)-one (1.92 g).

¹H-NMR (CDCl₃, δ ppm): 1.33 (6H, s), 2.31 (3H, s), 2.43-2.70 (1H, bs), 3.35 (2H, s), 4.42 (2H, s), 5.48 (2H, s), 6.21 (1H, s), 7.04-8.57 (12H, m).

### SYNTHESIS EXAMPLE 14

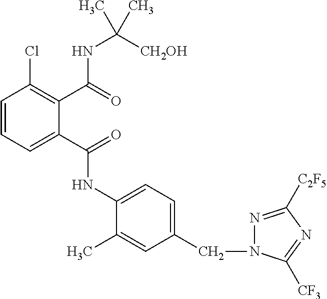

As similar to SYNTHESIS EXAMPLE 11, from N²-[2-(benzyloxy)-1,1-dimethylethyl]-3-chloro-N¹-(2-methyl-4-{[3-(pentafluoroethyl)-5-(trifluoromethyl)-1H-1,2,4-triazol-1-yl]methyl}phenyl)-1,2-benzenedicarboxamide (1.25 g) and 1 Mol/L solution (7 mL) of boron tribromide in dichloromethane, the objective compound 3-chloro-N²-(2-hydroxy-1,1-dimethylethyl)-N¹-(2-methyl-4-{[3-(pentafluoroethyl)-5-(trifluoromethyl)-1H-1,2,4-triazol-1-yl]methyl}phenyl)-1,2-benzenedicarboxamide (1.03 g) was obtained (compound No. 14-129).

Melting point: 105-110° C.

### SYNTHESIS EXAMPLE 15

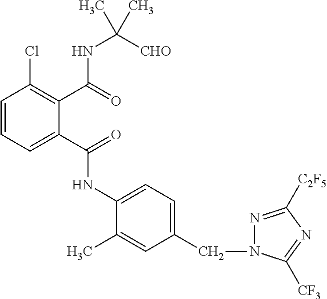

Oxalyl chloride (0.27 g) was added to a mixed solvent of dichloromethane (5 mL) and DMSO (0.3 mL) at −60° C., and the mixture was stirred for 10 minutes. Subsequently, 3-chloro-N²-(2-hydroxy-1,1-dimethylethyl)-N¹-(2-methyl-4-{[3-(pentafluoroethyl)-5-(trifluoromethyl)-1H-1,2,4-triazol-1-yl]methyl}phenyl)-1,2-benzenedicarboxamide (0.90 g) obtained from SYNTHESIS EXAMPLE 14 was added at −60° C., and after stirred at −60° C. for further 30 minutes, triethylamine (1 mL) was added, and the temperature was returned to room temperature. The reaction mixture was poured into water, extracted with dichloromethane, washed with saturated brine, and dried over anhydrous magnesium sulfate. The solvent was distilled off, and the crude product was subjected to silica gel column chromatography (mixed solvent of n-hexane and ethyl acetate) to obtain the objective compound 3-chloro-N²-(1,1-dimethyl-2-oxoethyl)-N¹-(2-methyl-4-{[3-(pentafluoroethyl)-5-(trifluoromethyl)-1H-1,2,4-triazol-1-yl]methyl}phenyl)-1,2-benzenedicarboxamide (0.44 g) (compound No. 14-130).

¹H-NMR (CDCl₃, δ ppm): 1.40 (6H, s), 2.29 (3H, s), 5.49 (2H, s), 6.63 (1H, s), 7.15-8.24 (7H, m), 9.37 (1H, s).

### SYNTHESIS EXAMPLE 16

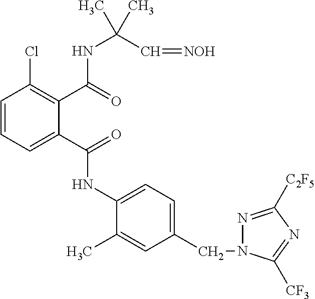

3-chloro-N²-(1,1-dimethyl-2-oxoethyl)-N¹-(2-methyl-4-{[3-(pentafluoroethyl)-5-(trifluoromethyl)-1H-1,2,4-triazol-1-yl]methyl}phenyl)-1,2-benzenedicarboxamide (0.36 g) obtained in SYNTHESIS EXAMPLE 15, hydroxylamine hydrochloride (0.05 g) and sodium acetate (0.09 g) were heated to reflux in ethanol for 5 hours. After the reaction was completed, the solvent was distilled off, and the crude product was subjected to silica gel column chromatography (mixed solvent of n-hexane and ethyl acetate) to obtain the objective compound 3-chloro-N²-[2-(hydroxyimino)-1,1-dimethylethyl]-N¹-(2-methyl-4-{[3-(pentafluoroethyl)-5-(trifluoromethyl)-1H-1,2,4-triazole-1-yl]methyl}phenyl)-1,2-benzenedicarboxamide (0.27 g) (compound No. 14-132).

Melting point: 203-206° C.

### SYNTHESIS EXAMPLE 17-1

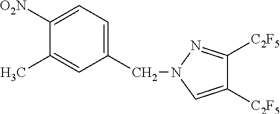

3-methyl-4-nitrobenzyl chloride (2.69 g), 3,4-bis(pentafluoroethyl)-1H-pyrazole (4.40 g), 18-crown-6 (0.19 g), tetrabutylammonium iodide (0.27 g), and potassium carbonate (3.00 g) were heated to reflux for 2 hours in acetonitrile (30 mL) for 2 hours. After the reaction was completed, water (100 mL) was added, and extracted with ethyl acetate. The organic phase was washed with saturated brine, and dried over anhydrous sodium sulfate. After the solvent was distilled off, the crude product was subjected to silica gel column chromatography (mixed solvent of n-hexane and ethyl acetate) to obtain 1-(3-methyl-4-nitrobenzyl)-3,4-bis(pentafluoroethyl)-1H-pyrazole (4.56 g).

¹H-NMR (CDCl₃, δ ppm): 2.61 (3H, s), 5.43 (2H, s), 7.18-7.28 (2H, m), 7.77 (1H, s), 8.00 (1H, d).

### SYNTHESIS EXAMPLE 17-2

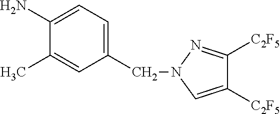

20% titanium trichloride aqueous solution (15.3 g) was added to a mixture of 1-(3-methyl-4-nitrobenzyl)-3,4-bis(pentafluoroethyl)-1H-pyrazole (1.00 g), ammonium acetate (17.00 g), acetone (30 mL) and water (17 mL) at room temperature, and stirred at room temperature for 12 hours. After the reaction was completed, the reaction mixture was extracted with ethyl acetate. The organic phase was washed with saturated brine and dried over anhydrous sodium sulfate. The solvent was distilled off to obtain 4-{[3,4-bis(pentafluoroethyl)-1,4-pyrazol-1-yl]methyl}-2-methylaniline (0.83 g).

¹H-NMR (CDCl₃, δ ppm): 2.17 (3H, s), 3.58-3.90 (2H, m), 5.22 (2H, s), 6.68 (1H, d), 6.96-7.04 (2H, m), 7.53 (1H, s).

### SYNTHESIS EXAMPLE 17-3

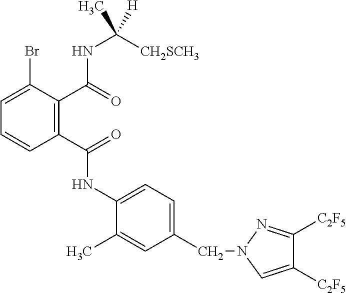

As similar to SYNTHESIS EXAMPLE 1-5, N¹-(4-{[3,4-bis(pentafluoroethyl)-1H-pyrazol-1-yl]methyl}-2-methylphenyl)-3-bromo-N²-[(1S)-1-methyl-2-(methylthio)ethyl]-1,2-benzenedicarboxamide (0.61 g) was obtained (compound No. 13-132) from 4-{[3,4-bis(pentafluoroethyl)-1H-pyrazol-1-yl]methyl}aniline (0.59 g) and 4-bromo-3-{[(1S)-1-methyl-2-(methylthio)ethyl]imino}-2-benzofuran-1(3H)-one (0.44 g).

Melting point: 82-86° C.

### SYNTHESIS EXAMPLE 17-4

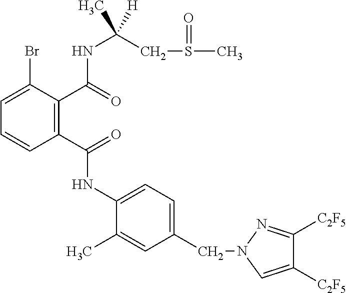

30% hydrogen peroxide (0.04 mL) was added to an acetic acid solution (0.27 mL) of N¹-(4-{[3,4-bis(pentafluoroethyl)-1H-pyrazol-1-yl]methyl}-2-methylphenyl)-3-bromo-N²-[(1S)-1-methyl-2-(methylthio)ethyl]-1,2-benzenedicarboxamide (0.21 g) at 10° C., and the mixture was further stirred at 10° C. for 3 hours. The reaction mixture was diluted with water and extracted with ethyl acetate. The organic phase was washed sequentially with a sodium thiosulfate aqueous solution and saturated sodium bicarbonate water, and dried over anhydrous sodium sulfate. After the solvent was distilled off, the crude product was subjected to silica gel column chromatography (mixed solvent of n-hexane and ethyl acetate) to obtain the objective compound N¹-(4-{[3,4-bis(pentafluoroethyl)-1H-pyrazol-1-yl]methyl}-2-methylphenyl)-3-bromo-N²-[(1S)-1-methyl-2-(methylsulfinyl)ethyl]-1,2-benzenedicarboxamide (0.15 g) (compound No. 13-137).

Melting point: 127-129° C.

### SYNTHESIS EXAMPLE 17-5

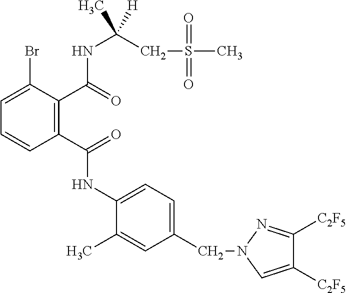

N¹-(4-{[3,4-bis(pentafluoroethyl)-1H-pyrazol-1-yl]methyl}-2-methylphenyl)-3-bromo-N²-[(1S)-1-methyl-2-(methylthio)ethyl]-1,2-benzenedicarboxamide (0.21 g) was dissolved in dichloromethane (10 mL), and after m-chloroperbenzoic acid (0.18 g) was added under ice cooling, the mixture was stirred at room temperature for 2 hours. After the reaction was completed, the reaction mixture was washed with saturated sodium bicarbonate water, a sodium thiosulfate aqueous solution and saturated brine, and dried over anhydrous sodium sulfate. The solvent was distilled off, and the resulting crude crystal was washed with petroleum ether to obtain N¹-(4-{[3,4-bis(pentafluoroethyl)-1H-pyrazol-1-yl]methyl}-2-methylphenyl)-3-bromo-N²-[(1S)-1-methyl-2-(methylsulfonyl)ethyl]-1,2-benzenedicarboxamide (0.21 g) (compound No. 13-140).

Melting point: 134-136° C.

### SYNTHESIS EXAMPLE 18-1

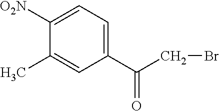

Bromine (1.00 g) was added to a diethyl ether solution (10 mL) of 3-methyl-4-nitroacetophenone (0.90 g) and anhydrous aluminum chloride (0.01 g) under ice cooling, and the mixture was stirred for one hour at the same temperature. After the reaction was completed, the solvent was distilled off, and the resulting crude product was subjected to silica gel column chromatography (mixed solvent n-hexane and ethyl acetate) to obtain 3-methyl-4-nitrophenacyl bromide (0.27 g).

¹H-NMR (CDCl₃, δ ppm): 2.65 (3H, s), 4.43 (2H, s), 7.89-8.05 (3H, m).

### SYNTHESIS EXAMPLE 18-2

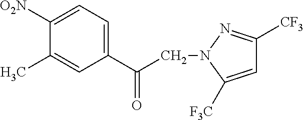

3-Methyl-4-nitrophenacyl bromide (0.57 g), 3,5-bis(trifluoromethyl)-1H-pyrazole (0.41 g) and triethylamine (0.24 g) were stirred in acetonitrile (10 mL) at room temperature for 2 hours. After the reaction was completed, the solvent was distilled off. The crude product was purified with silica gel column chromatography (mixed solvent n-hexane and ethyl acetate) to obtain 1-(3-methyl-4-nitropheny-2-[3,5-bis(trifluoromethyl)-1H-pyrazol-1-yl]ethanone (0.67 g).

¹H-NMR (CDCl₃, δ ppm): 2.67 (3H, s), 5.80 (2H, s), 7.03 (1H, s), 7.87-8.09 (3H, m).

### SYNTHESIS EXAMPLE 18-3

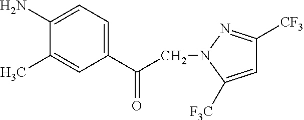

As similar to SYNTHESIS EXAMPLE 1-4, 1-(4-amino-3-methylphenyl)-2-[3,5-bis(trifluoromethyl)-1H-pyrazol-1-yl]ethanone (0.26 g) was obtained from 1-(3-methyl-4-nitrophenyl)-2-[3,5-bis(trifluoromethyl)-1H-pyrazol-1-yl]ethanone (0.52 g), tin (II) chloride dihydrate (1.52 g), concentrated hydrochloric acid (1.6 mL), and ethanol (2 mL).

¹H-NMR (CDCl₃, δ ppm): 2.32 (3H, s), 5.81 (2H, s), 7.02 (1H, s), 7.40 (1H, d), 7.73-8.01 (4H, m).

### SYNTHESIS EXAMPLE 18-4

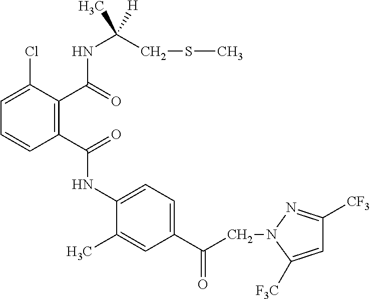

As similar to SYNTHESIS EXAMPLE 1-5, N¹-(4-{[3,5-bis(trifluoromethyl)]-1H-pyrazol-1-yl}acetyl)-2-methylphenyl)-3-chloro-N²-[(1S)-1-methyl-2-(methylthio)ethyl]-1,2-benzenedicarboxamide (0.40 g) was obtained (compound No. 13-365) from 4-chloro-3-{[(1S)-1-methyl-2-(methylthio)ethyl]imino}-2-benzofuran-1(3H)-one (0.19 g) and 1-(4-amino-3-methylphenyl)-2-[3,5-bis(trifluoromethyl)-1H-pyrazol-1-yl]ethanone (0.25 g).

¹H-NMR (CDCl₃, δ ppm): 1.24 (3H, d), 1.95 (3H, s), 2.45 (3H, s), 2.58 (2H, d), 4.30-4.41 (1H, m), 5.76 (2H, s), 6.12 (1H, d), 7.00 (1H, s), 7.50 (1H, dd), 7.60 (1H, d), 7.78-7.85 (3H, m), 8.53 (1H, d), 8.68 (1H, bs).

### SYNTHESIS EXAMPLE 18-5

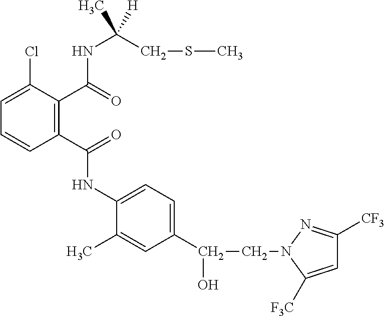

Sodium borohydride (0.02 g) was added to a methanol solution (10 mL) of N¹-(4-{[3,5-bis(trifluoromethyl)]-1H-pyrazol-1-yl}acetyl}-2-methylphenyl)-3-chloro-N²-[(1S)-1-methyl-2-(methylthio)ethyl]-1,2-benzenedicarboxamide (0.20 g) under ice cooling, and the mixture was stirred at room temperature for 3 hours. After the reaction was completed, water was added, and the precipitated crystal was collected by filtration, washed with water and dried to obtain N¹-(4-{2-[3,5-bis(trifluoromethyl)-1H-pyrazol-1-yl]-1-hydroxyethyl}-2-methylphenyl)-3-chloro-N²-[(1S)-1-methyl-2-(methylthio)ethyl]-1,2-benzenedicarboxamide (0.17 g) (compound No. 13-366).

¹H-NMR (CDCl₃, δ ppm): 1.26 (3H, d), 2.01 (3H, s), 2.34 (3H, s), 2.57 (1H, dd), 2.65 (1H, dd), 4.30-4.44 (3H, m), 5.22-5.31 (1H, m), 6.14 (1H, d), 6.92 (1H, s), 7.21-7.37 (2H, m), 7.47 (1H, dd), 7.56 (1H, d), 7.77 (1H, d), 8.10 (1H, d), 8.38 (1H, bs).

### SYNTHESIS EXAMPLE 19-1

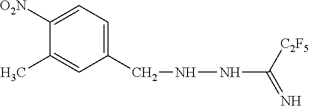

Pentafluoropropyl amidine (1.62 g) was added to a THF solution (15 mL) of 3-methyl-4-nitrobenzyl)-hydrazine (1.81 g), and the mixture was stirred at room temperature for 4 hours. After the reaction was completed, the solvent was distilled off, to obtain 2,2,3,3,3-pentafluoro-N′-(3-methyl-4-nitrobenzyl) propanimide hydrazide (2.90 g).

¹H-NMR (CDCl₃, δ ppm): 2.59 (3H, s), 4.32 (2H, s), 4.34-4.80 (3H, m), 7.24-7.35 (2H, m), 7.90 (1H, d).

### SYNTHESIS EXAMPLE 19-2

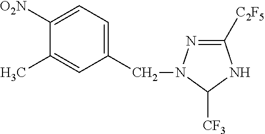

P-toluene sulfonic acid (0.02 g) was added to 2,2,3,3,3-pentafluoro-N′-(3-methyl-4-nitrobenzyl)-propaneimide hydrazide (2.47 g), and the mixture was heated to reflux for 2 hours while dehydrated in toluene. After cooled to room temperature, the solvent was distilled off, and the crude product was subjected to silica gel column chromatography (mixed solvent n-hexane and ethyl acetate) to obtain 1-(1-(3-methyl-4-nitrobenzyl)-3-(pentafluoroethyl)-5-(trifluoromethyl)-4,5-dihydro-1H-1,2,4-triazole (2.11 g).

¹H-NMR (CDCl₃, δ ppm): 2.59 (3H, s), 4.21 (1H, d), 4.52 (1H, d), 4.85-4.94 (1H, m), 5.13-5.22 (1H, m), 7.24-7.32 (2H, m), 7.96 (1H, d).

### SYNTHESIS EXAMPLE 19-3

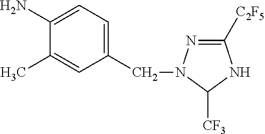

5% (w/w) palladium-carbon (0.05 g) was added to an ethanol solution (10 mL) of 1-(1-(3-methyl-4-nitrobenzyl)-3-(pentafluoroethyl)-5-(trifluoromethyl)-4,5-dihydro-1H-1,2,4-triazole (0.49 g), and the mixture was stirred at room temperature for 2 hours under hydrogen atmosphere. After the reaction was completed, palladium catalyst was filtered off with Celite. The filtrate was evaporated under reduced pressure to obtain 2-methyl-4-{[3-(pentafluoroethyl)-5-(trifluoromethyl)-4,5-dihydro-1H-1,2,4-triazol-1-yl]methyl}aniline (0.40 g).

¹H-NMR (CDCl₃, δ ppm): 2.14 (3H, s), 3.92 (1H, d), 4.46-4.54 (1H, m), 4.60 (1H, d), 5.10-5.19 (1H, m), 6.64 (1H, d), 6.90 (1H, d), 6.92 (1H, s).

### SYNTHESIS EXAMPLE 19-4

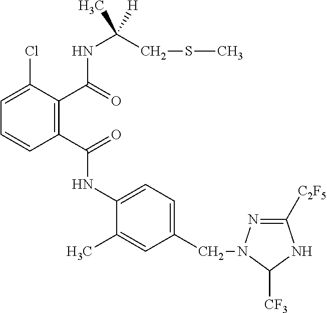

As similar to SYNTHESIS EXAMPLE 1-5, 3-chloro-N¹-(2-methyl-4-{[3-(pentafluoroethyl)-5-(trifluoromethyl)-4,5-dihydro-1H-1,2,4-triazol-1-yl]methyl}phenyl)-N²-[(1S)-1-methyl-2-(methylthio)ethyl]-1,2-benzenedicarboxamide as a diastereomer mixture (0.70 g) (compound No. 16-4) was obtained from 4-chloro-3-{[(1S)-1-methyl-2-(methylthio)ethyl]imino}-2-benzofuran-1(3H)-one (0.81 g) and 2-methyl-4-{[3-(pentafluoroethyl)-5-(trifluoromethyl)-4,5-dihydro-1H-1,2,4-triazole-1-yl]methyl aniline (1.13 g).

¹H-NMR (CDCl₃, δ ppm): 1.26 (3H, d), 2.02 (3H, s), 2.31 (3H, s), 2.40-2.65 (2H, m), 3.96-4.07 (1H, m), 4.23-4.40 (1H, m), 4.58-4.70 (1H, m), 4.81-4.94 (1H, m), 5.07-5.18 (1H, m), 6.18 (1H, bs), 7.06-7.17 (2H, m), 7.42-8.13 (4H, m), 8.34 (1H, bs).

### SYNTHESIS EXAMPLE 19-5

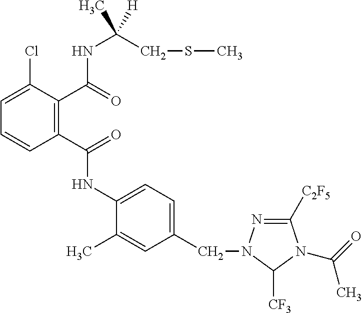

Acetic anhydride (0.08 g) was added to a THF solution (5 mL) of 3-chloro-N¹-(2-methyl-4-{[3-(pentafluoroethyl)-5-(trifluoromethyl)-4,5-dihydro-1H-1,2,4-triazole-1-yl]methyl}phenyl)-N²-[(1S)-1-methyl-2-(methylthio)ethylk 1,2-benzenedicarboxamide (0.17 g) and triethylamine (0.08 g) under ice cooling, and the mixture was stirred for 3 hours while the temperature was gradually returned to room temperature. After the reaction was completed, water was added, and the reaction mixture was extracted with ethyl acetate. The organic phase was washed with saturated brine (100 mL) and dried over anhydrous sodium sulfate. The solvent was distilled off, and the resulting crude product was subjected to silica gel column chromatography (mixed solvent n-hexane and ethyl acetate) to obtain N¹-(4-{[4-acetyl-3-(pentafluoroethyl)-5-(trifluoromethyl)-4,5-dihydro-1H-1,2,4-triazol-1-yl]methyl}-2-methylphenyl)-3-chloro-N²-[(2S)-1-(methylsulfanyl)propan-2-yl]-1,2-benzenedicarboxamide (0.086 g) (compound No. 16-6).

¹H-NMR (CDCl₃, δ ppm): 1.24 (3H, d), 2.00 (3H, s), 2.13 (3H, s), 2.52-2.68 (2H, m), 4.22 (1H, d), 4.29-4.41 (1H, m), 4.59 (1H, d), 5.64-5.71 (1H, m), 6.12 (1H, d), 7.03-7.13 (2H, m), 7.45-7.79 (3H, m), 8.13 (1H, d), 8.36 (1H, bs).

### SYNTHESIS EXAMPLE 20-1

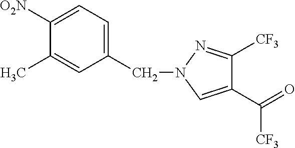

As similar to SYNTHESIS EXAMPLE 1-3, 1-(3-methyl-4-nitrobenzyl)-4-(trifluoroacetyl)-3-(trifluoromethyl)-1H-pyrazole (0.61 g) was obtained from 3-methyl-4-nitrobenzyl chloride (0.80 g), 4-(trifluoroacetyl)-3-(trifluoromethyl)-1H-pyrazole (0.90 g) and potassium carbonate (0.72 g).

¹H-NMR (CDCl₃, δ ppm): 2.62 (3H, s), 5.45 (2H, s), 7.27 (1H, d), 7.29 (1H, s), 8.01 (1H, d), 8.13 (1H, s).

### SYNTHESIS EXAMPLE 20-2

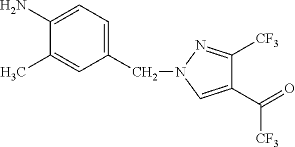

As similar to SYNTHESIS EXAMPLE 17-2, 20% titanium trichloride aqueous solution (9.03 g) was added to a mixture of 1-(3-methyl-4-nitrobenzyl)-4-(trifluoroacetyl)-3-(trifluoromethyl)-1H-pyrazole (0.61 g), ammonium acetate (10.03 g), acetone (20 mL) and water (20 mL), and 4-{[4-(trifluoroacetyl)-3-(trifluoromethyl)-1H-pyrazol-1-yl]methyl}-2-methylaniline (0.32 g) was obtained.

¹H-NMR (CDCl₃, δ ppm): 2.17 (3H, s), 3.64-3.94 (2H, m), 5.23 (2H, s), 6.67 (1H, d), 6.99-7.06 (2H, m), 7.91 (1H, s).

### SYNTHESIS EXAMPLE 20-3

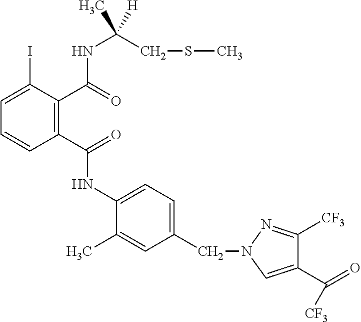

As similar to SYNTHESIS EXAMPLE 1-5, 3-iodo-N¹-(2-methyl-4-{[4-(trifluoroacetyl)-3-(trifluoromethyl)-1H-pyrazol-1-yl]methyl}phenyl)-N²-[(1S)-1-methyl-2-(methylthio)ethyl]-1,2-benzenedicarboxamide (0.18 g) was obtained (compound No. 13-507) from 4-iodo-3-{[(1S)-1-methyl-2-(methylthio)ethyl]imino}-2-benzofuran-1(3H)-one (0.38 g) and 4-{[4-(trifluoroacetyl)-3-(trifluoromethyl)-1H-pyrazol-1-yl]methyl}-2-methylaniline (0.32 g).

¹H-NMR (CDCl₃, δ ppm): 1.25 (3H, d), 1.92 (3H, s), 2.15 (3H, s), 2.54 (1H, dd), 2.62 (1H, dd), 4.23-4.37 (1H, m), 5.34 (2H, s), 6.47 (1H, d), 7.12-7.22 (3H, m), 7.72 (1H, d), 7.93 (1H, d), 8.04 (1H, s), 8.17 (1H, d), 8.46 (1H, s).

### SYNTHESIS EXAMPLE 20-4

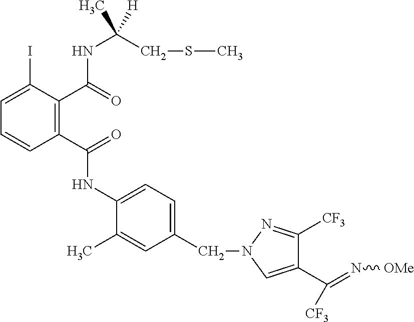

3-iodo-N¹-(2-methyl-4-{[4-(trifluoroacetyl)-3-(trifluoromethyl)-1H-pyrazol-1-yl]methyl}phenyl)-N²-[(1S)-1-methyl-2-(methylthio)ethyl]-1,2-benzenedicarboxamide (0.10 g) and O-methylhydroxylamine hydrochloride (0.013 g) were heated and stirred in a mixed solvent of pyridine (4 mL) and ethanol (1 mL) at 50° C. for 2 hours. After the reaction was completed, the solvent was distilled off, and the crude product was subjected to silica gel column chromatography (mixed solvent of n-hexane and ethyl acetate) to obtain an E/Z mixture of 3-iodo-N²-[(1S)-1-methyl-2-(methylthio)ethyl]-N¹-[2-methyl-4-({4-[2,2,2-trifluoro-N-methoxyethanimidoyl]-3-(trifluoromethyl)-1H-pyrazol-1-yl}methyl)phenyl]-1,2-benzenedicarboxamide (0.04 g) (compound No. 13-147).

¹H-NMR (CDCl₃, δ ppm): 1.25-1.26 (3H, m), 1.93 (3H, s), 2.32 (3H, s), 2.56-2.60 (2H, m), 4.01-4.07 (3H, m), 4.32-4.32 (1H, m), 5.21-5.26 (2H, m), 6.17 (1H, d), 7.16-7.22 (2H, m), 7.40-7.50 (2H, m), 7.79 (1H, d), 7.96-7.99 (1H, m), 8.19 (1H, d), 8.34 (1H, s).

The synthesis examples of starting materials will be illustrated as follows.

### SYNTHESIS EXAMPLE 21-1

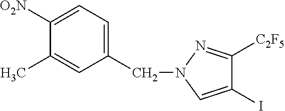

As similar to SYNTHESIS EXAMPLE 1-3, 4-iodo-1-(3-methyl-4-nitrobenzyl)-3-(pentafluoroethyl)-1H-pyrazole (4.60 g) was obtained from 3-methyl-4-nitrobenzyl chloride (8.56 g), 4-iodo-3-(pentafluoroethyl)-1H-pyrazole (16.00 g), and potassium carbonate (7.66 g).

¹H-NMR (CDCl₃, δ ppm): 2.59 (3H, s), 5.38 (2H, s), 7.14-7.22 (2H, m), 7.53 (1H, s), 7.97 (1H, d)

### SYNTHESIS EXAMPLE 21-2

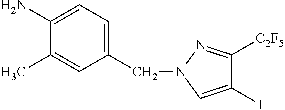

As similar to SYNTHESIS EXAMPLE 1-4, 4-{[4-iodo-3-(pentafluoroethyl)-1H-pyrazol-1-yl]methyl}aniline (1.78 g) was obtained from 4-iodo-1-(3-methyl-4-nitrobenzyl)-3-(pentafluoroethyl)-1H-pyrazole (3.00 g), tin (II) chloride dihydrate (3.67 g), concentrated hydrochloric acid (1 mL) and ethanol (10 mL).

¹H-NMR (CDCl₃, δ ppm): 2.16 (3H, s), 3.71 (2H, bs), 5.19 (2H, s), 6.66 (1H, d), 6.95-7.02 (2H, m), 7.34 (1H, s).

### SYNTHESIS EXAMPLE 21-3

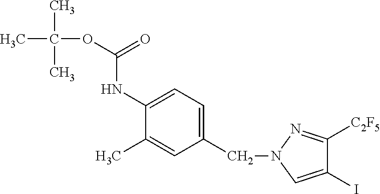

A toluene solution (10 mL) of 4-{[4-iodo-3-(pentafluoroethyl)-1H-pyrazol-1-yl]methyl}aniline (1.80 g), and di-t-butyl dicarbonate (1.37 g) was heated to reflux for 2 hours. Water (3 mL) was added to the reaction mixture, and further heated to reflux for 15 minutes. After cooled to room temperature, the extraction was made with diethyl ether. The organic phase was washed with saturated brine, and then dried over anhydrous magnesium sulfate. After the solvent was distilled off, the obtained crude crystal is washed with n-hexanet to obtain t-butyl (4-{[4-iodo-3-(pentafluoroethyl)-1H-pyrazol-1-yl]methyl}-2-methylphenyl)carbamate (1.56 g).

¹H-NMR (CDCl₃, δ ppm): 1.53 (9H, s), 2.24 (3H, s), 5.24 (2H, s), 6.30 (1H, bs), 7.06 (1H, bs), 7.12 (1H, d), 7.37 (1H, s), 7.89 (1H, d).

### SYNTHESIS EXAMPLE 21-4

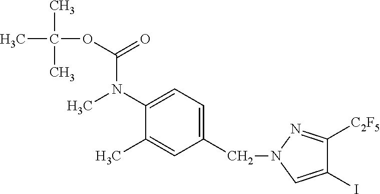

t-Butyl (4-{[4-iodo-3-(pentafluoroethyl)-1H-pyrazol-1-yl]methyl}-2-methylphenyl)carbamate (1.56 g) and methyl iodide (0.83 g) were added to a THF suspension (10 mL) of sodium hydride (0.14 g, 60% oiliness), and the mixture was stirred at room temperature for 1 hour. A small amount of water was added to the reaction mixture, which was then diluted with ethyl acetate. The organic phase was separated, washed with water and saturated brine, and dried over anhydrous magnesium sulfate. After the solvent was distilled off, the obtained crude product was purified with silica gel column chromatography (mixed solvent of n-hexane and ethyl acetate) to obtain t-butyl (4-{[4-iodo-3-(pentafluoroethyl)-1H-pyrazol-1-yl]methyl}-2-methylphenyl)methylcarbamate (1.60 g).

¹H-NMR (CDCl₃, δ ppm): 1.33 (9H, s), 2.21 (3H, s), 3.14 (3H, s), 5.29 (2H, s), 6.98-7.22 (3H, m).

### SYNTHESIS EXAMPLE 21-5

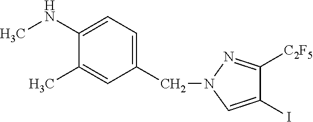

Trifluoroacetic acid (1.67 g) was added to a dichloromethane solution (10 mL) of t-butyl (4-{[4-iodo-3-(pentafluoroethyl)-1H-pyrazol-1-yl]methyl}-2-methylphenyl)carbamate (1.60 g), and the mixture was stirred at room temperature for 5 hours. The solvent was distilled off, and the resulting crude product was diluted with ethyl acetate, washed with a saturated aqueous solution of sodium hydrogen carbonate and saturated brine, and dried over anhydrous magnesium sulfate. After the solvent was distilled off, 4-{[4-iodo-3-(pentafluoroethyl)-1H-pyrazol-1-yl]methyl}-N,2-dimethylaniline (1.30 g) was obtained.

¹H-NMR (CDCl₃, δ ppm): 2.12 (3H, s), 2.91 (3H, s), 5.19 (2H, s), 6.58 (1H, d), 6.99 (1H, s), 7.10 (1H, d), 7.33 (1H, s).

### SYNTHESIS EXAMPLE 21-6

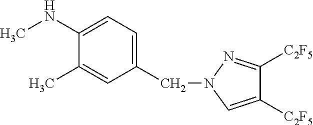

4-{[4-iodo-3-(pentafluoroethyl)-1H-pyrazol-1-yl]methyl}-N,2-dimethylaniline (1.20 g), copper powder (0.51 g), iodopentafluoroethane (1.33 g) and DMSO (10 mL) were charged into an autoclave, and heated and stirred at 120° C. for 8 hours. After cooled to room temperature, the reaction mixture was poured into iced water and diluted with ethyl acetate (50 mL), and then washed with ethyl acetate after an insoluble material was filtered off with Celite. After dried over anhydrous magnesium sulfate, the filtrate was concentrated under reduced pressure, and the resulting crude product was purified with silica gel column chromatography (mixed solvent of n-hexane and ethyl acetate) to obtain 4-{[3,4-bis(pentafluoroethyl)-1H-pyrazol-1-yl]methyl}-N,2-dimethylaniline (0.47 g).

¹H-NMR (CDCl₃, δ ppm): 2.14 (3H, s), 2.90 (3H, s), 3.74 (1H, bs), 5.21 (2H, s), 6.60 (1H, d), 7.01 (1H, s), 7.11 (1H, d), 7.52 (1H, s).

### SYNTHESIS EXAMPLE 22

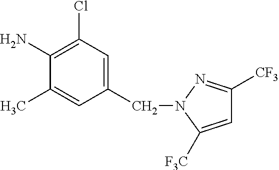

N-chlorosuccinimide (0.28 g) was added to a DMF solution (8 mL) of 4-{[3,5-bis(trifluoromethyl-1H-pyrazol-1-yl]methyl}-2-methylaniline (0.64 g), and the mixture was heated and stirred at 60° C. for 2 hours. After cooled to room temperature, and water was added, the reaction mixture was extracted with dichloromethane and dried over anhydrous sodium sulfate. After the solvent was distilled off, the resulting crude product was purified with silica gel column chromatography (mixed solvent of n-hexane and ethyl acetate) to obtain 4-{[3,5-bis(trifluoromethyl)-1H-pyrazol-1-yl]methyl}-2-chloro-6-methylaniline (0.33 g).

¹H-NMR (CDCl₃, δ ppm): 2.17 (3H, s), 4.07 (2H, bs), 5.27 (2H, s), 6.87 (1H, s), 6.91 (1H, bs), 7.09 (1H, bs).

### SYNTHESIS EXAMPLE 23-1

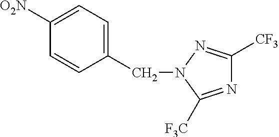

As similar to SYNTHESIS EXAMPLE 8-1, 1-(4-nitrobenzyl)-3,5-bis(trifluoromethyl)-1H-1,2,4-triazole (9.40 g) was obtained from 4-nitrobenzyl chloride (6.48 g), 3,5-bis(trifluoromethyl)-1H-1,2,4-triazole (6.15 g), potassium carbonate (6.22 g), 18-crown-6 (0.40 g) and tetrabutyl ammonium iodide (0.55 g).

¹H-NMR (CDCl₃, δ ppm): 5.63 (2H, s), 7.50 (2H, d), 8.27 (2H, d).

### SYNTHESIS EXAMPLE 23-2

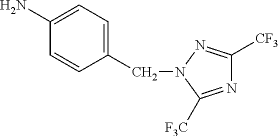

As similar to SYNTHESIS EXAMPLE 1-4, 4-{[3,5-bis(trifluoromethyl)-1H-1,2,4-triazol-1-yl]methyl}aniline (7.64 g) was obtained from 1-(4-nitrobenzyl)-3,5-bis(trifluoromethyl)-1H-1,2,4-triazole (9.40 g), tin (II) chloride dihydrate (31.18 g), concentrated hydrochloric acid (32 mL) and ethanol (50 mL).

¹H-NMR (CDCl₃, δ ppm): 3.36-3.95 (2H, m), 5.39 (2H, s), 6.65 (2H, d), 7.16 (2H, d).

### SYNTHESIS EXAMPLE 23-3

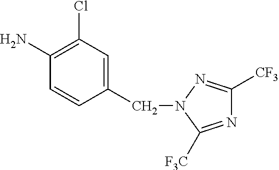

As similar to SYNTHESIS EXAMPLE 22, 4-{[3,5-bis(trifluoromethyl)-1H-1,2,4-triazol-1-yl]methyl}-2-chloroaniline (0.07 g) was obtained from 4-{[3,5-bis(trifluoromethyl)-1H-1,2,4-triazol-1-yl]methyl}aniline (0.35 g) and N-chlorosuccinimide (0.14 g).

¹H-NMR (CDCl₃, δ ppm): 4.10-4.39 (2H, m), 5.37 (2H, s), 6.75 (1H, d), 7.06 (1H, dd), 7.26 (1H, d).

### SYNTHESIS EXAMPLE 24

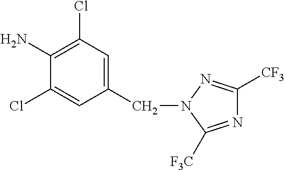

As similar to SYNTHESIS EXAMPLE 22, 4-{[3,5-bis(trifluoromethyl)-1H-1,2,4-triazol-1-yl]methyl}-2,6-dichloroaniline (0.26 g) was obtained from 4-{[3,5-bis(trifluoromethyl)-1H-1,2,4-triazol-1-yl]methyl}aniline (0.80 g) and N-chlorosuccinimide (0.72 g).

¹H-NMR (CDCl₃, δ ppm): 4.35-4.96 (2H, m), 5.36 (2H, s), 7.21 (2H, s).

### SYNTHESIS EXAMPLE 25

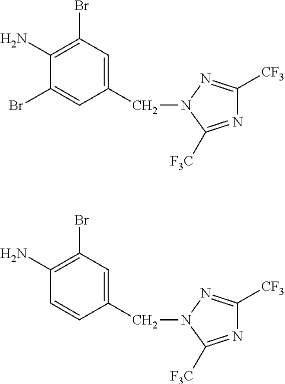

As similar to SYNTHESIS EXAMPLE 22, 4-{[3,5-bis(trifluoromethyl)-1H-1,2,4-triazol-1-yl]methyl}aniline (0.50 g) was reacted with N-bromosuccinimide (0.30 g) and subjected to silica gel column chromatography (mixed solvent of n-hexane and ethyl acetate) to obtain, as the first elution portion, 4-{[3,5-bis(trifluoromethyl)-1H-1,2,4-triazol-1-yl]methyl}-2, 6-bromoaniline (0.07 g),

¹H-NMR (CDCl₃, δ ppm): 4.71 (2H, bs), 5.34 (2H, s), 7.40 (2H, s); and as the second elution portion, 4-{[3,5-bis(trifluoromethyl)-1H-1,2,4-triazol-1-yl]methyl}-2-bromoaniline (0.20 g).

¹H-NMR (CDCl₃, δ ppm): 5.39 (2H, s), 5.55-5.85 (2H, m), 6.75 (1H, d), 7.11 (1H, dd), 7.44 (1H, d).

### SYNTHESIS EXAMPLE 26-1

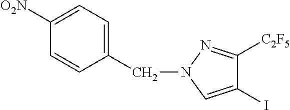

As similar to SYNTHESIS EXAMPLE 1-3, 4-iodo-1-(4-nitrobenzyl)-3-(pentafluoroethyl)-1H-pyrazole (3.00 g) was obtained from 4-nitrobenzyl chloride (3.46 g), 4-iodo-3-(pentafluoroethyl)-1H-pyrazole (5.00 g) and potassium carbonate (2.66 g).

¹H-NMR (CDCl₃, δ ppm): 5.45 (2H, s), 7.38 (2H, d), 7.55 (1H, s), 8.24 (2H, s).

### SYNTHESIS EXAMPLE 26-2

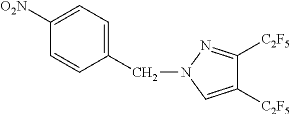

4-iodo-1-(4-nitrobenzyl)-3-(pentafluoroethyl)-1H-pyrazole (2.50 g), copper powder (1.07 g), iodopentafluoroethane (4.13 g) and DMSO (10 mL) were charged into an autoclave, and heated and stirred at 120° C. for 8 hours. After cooled to room temperature, the reaction mixture was poured into iced water, diluted with ethyl acetate (50 mL), and washed with ethyl acetate after an insoluble matter was filtered off with Celite. After dried over anhydrous magnesium sulfate, the filtrate was concentrated under reduced pressure, and the resulting crude product was purified with silica gel column chromatography to obtain 1-(4-nitrobenzyl)-3,4-bis(pentafluoroethyl)-1H-pyrazole (1.54 g).

¹H-NMR (CDCl₃, δ ppm): 5.50 (2H, s), 7.43 (2H, d), 7.78 (1H, s), 8.27 (2H, s).

### SYNTHESIS EXAMPLE 26-3

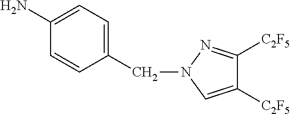

As similar to SYNTHESIS EXAMPLE 17-2, 4-{[3,4-bis(pentafluoroethyl)-1H-pyrazol-1-yl]methyl}aniline (0.64 g) was obtained from 1-(4-nitrobenzyl)-3,4-bis(pentafluoroethyl)-1H-pyrazole (1.54 g), ammonium acetate (27.03 g), acetone (20 mL), water (20 mL) and 20% titanium trichloride aqueous solution (24.34 g).

¹H-NMR (CDCl₃, δ ppm): 3.62-3.95 (2H, m), 5.22 (2H, s), 6.69 (2H, d), 7.11 (2H, d), 7.53 (1H, s).

### SYNTHESIS EXAMPLE 26-4

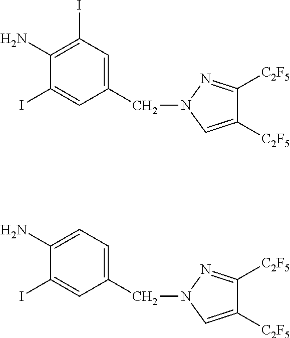

An acetic acid solution (5 mL) of iodine monocholoride (0.35 g) was added dropwise into an acetic acid solution (5 mL) of 4-{[3,4-bis(pentafluoroethyl)-1H-pyrazol-1-yl]methyl}aniline (0.42 g) at room temperature for 15 minutes, and the mixture was further stirred at room temperature for 2 hours. After acetic acid was distilled off under reduced pressure, water, dichloromethane and sodium hydrogen carbonate were added to the crude product, and the organic phase was separated, washed sequentially with saturated aqueous solution of sodium hydrogen carbonate and saturated brine, and dried over anhydrous magnesium sulfate. The solvent was distilled off, and the resulting crude product was purified with silica gel column chromatography (mixed solvent of n-hexane and ethyl acetate) to obtain, as the first elution portion, 4-{[3,4-bis(pentafluoroethyl)-1H-pyrazol-1-yl]methyl}-2,6-diiodoaniline (0.26 g);

¹H-NMR (CDCl₃, δ ppm): 4.67-4.87 (2H, m), 5.15 (2H, s), 7.62 (2H, s), 7.63 (1H, s);

and, as the second elution portion, 4-{[3,4-bis(pentafluoroethyl)-1H-pyrazol-1-yl]methyl}-2-iodoaniline (0.10 g).

¹H-NMR (CDCl₃, δ ppm): 3.72-4.50 (2H, m), 5.19 (2H, s), 6.74 (1H, d), 7.09 (1H, dd), 7.59 (1H, s), 7.63 (1H, d).

### SYNTHESIS EXAMPLE 27-1

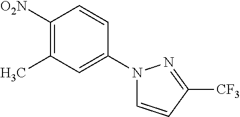

Potassium carbonate (1.66 g) was added to a DMF solution (20 mL) of 5-fluoro-2-nitrotoluene (1.55 g) and 3-(trifluoromethyl)-1H-pyrazole (1.36 g), and the mixture was stirred at 140° C. for 4 hours. After the reaction mixture was poured into water, the precipitated crude crystal was collected by filtration, washed with water and petroleum ether and dried to obtain 1-(3-methyl-4-nitrophenyl)-3-(trifluoromethyl)-1H-pyrazole (1.00 g).

¹H-NMR (CDCl₃, δ ppm): 2.72 (3H, s), 6.79 (1H, d), 7.69 (1H, dd), 7.79 (1H, d), 8.05 (1H, d), 8.16 (1H, d).

### SYNTHESIS EXAMPLE 27-2

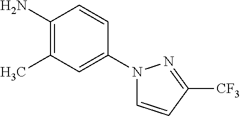

As similar to SYNTHESIS EXAMPLE 1-4, 2-methyl-4-[3-(trifluoromethyl)-1H-pyrazol-1-yl]aniline (0.95 g) was obtained from 1-(3-methyl-4-nitrophenyl-3-(trifluoromethyl)-1H-pyrazole (1.36 g), tin (II) chloride dihydrate (5.64 g), concentrated hydrochloric acid (5.8 mL) and ethanol (10 mL).

¹H-NMR (CDCl₃, δ ppm): 2.21 (3H, s), 3.64-3.81 (2H, m), 6.65 (1H, d), 6.71 (1H, d), 7.27 (1H, dd), 7.39 (1H, d), 7.77-7.80 (1H, m).

### SYNTHESIS EXAMPLE 28-1

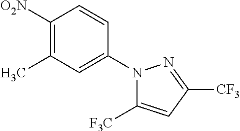

As similar to SYNTHESIS EXAMPLE 20-1, 1-(3-methyl-4-nitrophenyl)-3,5-bis(trifluoromethyl)-1H-pyrazole (1.74 g) was obtained from 5-fluoro-2-nitrotoluene (0.93 g), 3,5-bis(trifluoromethyl)-1H-pyrazole (1.22 g), potassium carbonate (1.00 g) and a DMF solution (12 mL).

¹H-NMR (CDCl₃, δ ppm): 2.69 (3H, s), 7.14 (1H, s), 7.50-7.61 (2H, m), 8.13 (1H, d).

### SYNTHESIS EXAMPLE 28-2

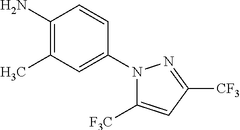

As similar to SYNTHESIS EXAMPLE 1-4, 4-[3,5-bis(trifluoro-methyl)-1H-pyrazol-1-yl]-2-methylaniline (1.40 g) was obtained from 1-(3-methyl-4-nitrophenyl)-3,5-bis(trifluoromethyl)-1H-pyrazole (1.74 g), tin (II) chloride dihydrate (5.79 g), concentrated hydrochloric acid (6.0 mL) and ethanol (30 mL).

¹H-NMR (CDCl₃, δ ppm): 2.18 (3H, s), 3.69-4.03 (2H, m), 6.69 (1H, d), 7.00 (1H, s), 7.07-7.16 (2H, m).

### SYNTHESIS EXAMPLE 29-1

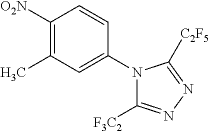

After 1,4-dichloro-1,4-bis(pentafluoroethyl)-1,3-diazatetra-1,3-diene (3.80 g) and triethylamine (2.13 g) were added to a THF solution (150 mL) of 4-nitro-3-methylaniline (1.44 g), the mixture was stirred at room temperature for 8 hours. Triethylamine (2.13 g) was then added, and the mixture was further heated to reflux for 4 hours. After cooled to room temperature, the solvent was distilled off, and the crude product was subjected to silica gel column chromatography (mixed solvent of n-hexane and ethyl acetate) to obtain 4-(3-methyl-4-nitrophenyl)-3,5-bis(pentafluoroethyl)-4H-1,2,4-triazole (0.33 g).

¹H-NMR (CDCl₃, δ ppm): 2.69 (3H, s), 7.14 (1H, s), 7.50-7.61 (2H, m), 8.13 (1H, d).

### SYNTHESIS EXAMPLE 29-2

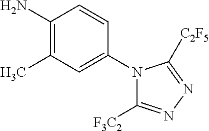

Nickel chloride (2) hexahydrate (0.090 g) was added to methanol (10 mL), and sodium borohydride (0.043 g) was added thereto under ice cooling. After the temperature was returned to room temperature, 4-(3-methyl-4-nitrophenyl)-3,5-bis(pentafluoroethyl)-4H-1,2,4-triazole (0.33 g) was added and the mixture was stirred for 1 hour. After the solvent was distilled off, 1N hydrochloric acid (10 mL) and ammonium chloride aqueous solution (10 mL) were added to the crude product, and the extraction was made with ethyl acetate. After the drying, the solvent was distilled off, and the crude product was subjected to silica gel column chromatography (mixed solvent of n-hexane and ethyl acetate) to obtain 4-[3,5-bis(pentafluoroethyl)-4H-1,2,4-triazol-4-yl]-2-methylaniline (0.26 g).

¹H-NMR (CDCl₃, δ ppm): 2.23 (3H, s), 3.90-4.10 (2H, m), 6.70 (1H, d), 6.95-7.08 (2H, m).

### SYNTHESIS EXAMPLE 30-1

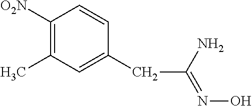

A methanol solution (50 mL) of (3-methyl-4-nitrophenyl)acetonitrile (8.81 g), hydroxylamine hydrochloride (4.52 g) and triethylamine (6.58 g) was stirred at room temperature for 8 hours. Then, the solvent was distilled off, and water was added to the crude product, which was then extracted with ethyl acetate. The organic phase was washed with saturated brine and dried over anhydrous sodium sulfate, the solvent was distilled off to obtain crude 2-(3-methyl-4-nitrophenyl)acetamide oxime (6.30 g).

¹H-NMR (CDCl₃, δ ppm): 2.48 (3H, s), 3.36 (2H, bs), 5.51 (2H, s), 7.28-7.40 (2H, m), 7.92 (1H, d), 8.95-9.05 (1H, m).

### SYNTHESIS EXAMPLE 30-2

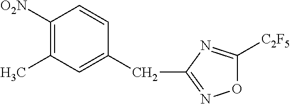

A toluene solution (10 mL) of a pentafluoropropionic anhydride (8.89 g) was added dropwise into a toluene solution (40 mL) of 2-(3-methyl-4-nitrophenyl)acetamide oxime (2.00 g) for 30 minutes while being heated to reflux, and then further heated to reflux for 4 hours. After cooled to room temperature, water and ethyl acetate were added. The organic phase was separated and dried over anhydrous sodium sulfate. The solvent was distilled off, and the crude product was subjected to silica gel column chromatography (mixed solvent of n-hexane and ethyl acetate) to obtain 3-(3-methyl-4-nitrobenzyl)-5-(pentafluoroethyl)-1,2,4-oxadiazole (2.45 g).

¹H-NMR (CDCl₃, δ ppm): 2.62 (3H, s), 4.24 (2H, s), 7.30-7.35 (2H, m), 7.98 (1H, d).

### SYNTHESIS EXAMPLE 30-3

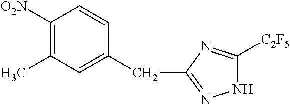

Hydrazine hydrate was added to a methanol solution (50 mL) of 3-(3-methyl-4-nitrobenzyl)-5-(pentafluoroethyl)-1,2,4-oxadiazole (2.40 g) at room temperature, and the mixture was stirred for 48 hours. After the solvent was distilled off, water was added to the crude product, which was then extracted with ethyl acetate. After the organic phase was washed with saturated brine and dried over anhydrous sodium sulfate, the solvent was distilled off, and the crude product was subjected to silica gel column chromatography (mixed solvent of n-hexane and ethyl acetate) to obtain 3-(3-methyl-4-nitrobenzyl)-5-(pentafluoroethyl)-1,2,4-triazole (1.89 g).

¹H-NMR (CDCl₃, δ ppm): 2.57 (3H, s), 4.30 (2H, s), 7.22-7.29 (2H, m), 7.96 (1H, d), 11.41-11.69 (1H, m).

### SYNTHESIS EXAMPLE 30-4

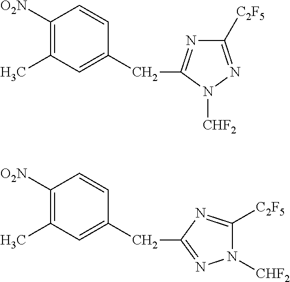

5-(3-methyl-4-nitrobenzyl)-3-(pentafluoroethyl)-1H-1,2,4-triazole (1.89 g), chlorodifluoromethane (1.94 g), potassium carbonate (1.16 g) and DMF (10 mL) were added into a glass pressure-resistant vessel, and the mixture was heated and stirred at 90° C. for 12 hours. After cooled to room temperature, water was added, the reaction mixture was extracted with ethyl acetate. The organic phase was washed with water and dried over anhydrous sodium sulfate. The solvent was then distilled off, and the crude product was subjected to silica gel column chromatography (mixed solvent of n-hexane and ethyl acetate) to obtain 1-(difluoromethyl)-5-(3-methyl-4-nitrobenzyl)-3-(pentafluoroethyl)-1H-1,2,4-triazole and 1-(difluoromethyl)-3-(3-methyl-4-nitrobenzyl)-5-(pentafluoroethyl)-1H-1,2,4-triazole as a mixture (1.40 g) at an approximate ratio of 1:1, which was subjected to the next reaction without further purification.

¹H-NMR (CDCl₃, δ ppm): 2.59 (3H, s), 4.20 and 4.53 (2H, s), 7.23-7.37 (3H, m), 7.95 and 7.97 (1H, d).

### SYNTHESIS EXAMPLE 30-5

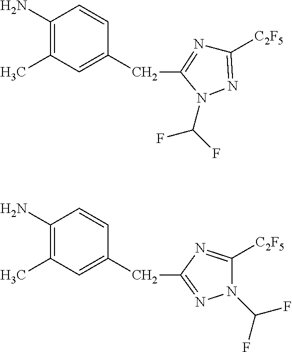

20% titanium trichloride aqueous solution (24.1 g) was added to a mixture of acetic acid ammonium (2.67 g), acetone (40 mL) and water (27 mL) and a mixture (1.34 g) of 1-(difluoromethyl)-5-(3-methyl-4-nitrobenzyl)-3-(pentafluoroethyl)-1H-1,2,4-triazole and 1-(difluoromethyl)-3-(3-methyl-4-nitrobenzyl)-5-(pentafluoroethyl)-1H-1,2,4-triazole at an approximate ratio of 1:1 at room temperature, and the reaction mixture was stirred at room temperature for 8 hours. After the reaction was completed, the reaction mixture was extracted with dichloromethane, washed with saturated aqueous solution of sodium hydrogen carbonate and saturated brine and dried over anhydrous sodium sulfate. After the solvent was distilled off, the crude product was subjected to silica gel column chromatography (mixed solvent of n-hexane and ethyl acetate) to obtain, as the first elution portion, 4-{[1-(difluoromethyl)-3-(pentafluoroethyl)-1H-1,2,4-triazol-5-yl]methyl}-2-methylaniline (0.29 g);

¹H-NMR (CDCl₃, δ ppm): 2.14 (3H, s), 3.50-3.66 (2H, m), 4.01 (2H, s), 6.62 (1H, d), 6.95-7.05 (2H, m), 7.30 (1H, t);

and, as the second elution portion, 4-{[1-(difluoromethyl)-5-(pentafluoroethyl)-1H-1,2,4-triazol-3-yl]methyl}-2-methylaniline (0.40 g).

¹H-NMR (CDCl₃, δ ppm): 2.13 (3H, s), 3.50-3.70 (2H, m), 4.23 (2H, s), 6.62 (1H, d), 6.88-6.97 (2H, m), 7.22 (1H, t).

### SYNTHESIS EXAMPLE 31-1

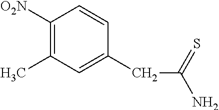

Hydrogen sulfide gas was blown into a pyridine solution of (3-methyl-4-nitrophenyl)acetonitrile (3.52 g) at room temperature for 3 hours. The reaction mixture was poured into ice, and the precipitated crystal was collected by filtration with suction, washed with water and dried to obtain 2-(3-methyl-4-nitrophenyl)thioacetamide (1.69 g).

¹H-NMR (CDCl₃, δ ppm): 2.60 (3H, s), 4.06 (2H, s), 6.40-8.00 (5H, m).

### SYNTHESIS EXAMPLE 31-2

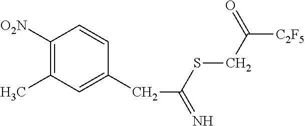

A mixture of 2-(3-methyl-4-nitrophenyl)thioacetamide (1.00 g), 1-bromo-3,3,4,4,4-pentafluoro-2-butanone (1.15 g) and potassium carbonate (0.79 g) was stirred in DMF (10 mL) at room temperature for 1 hour. The reaction mixture was diluted with ethyl acetate, washed with water and saturated brine, and dried over magnesium sulfate. The solvent was distilled off, and the resulting crude product was purified with silica gel column chromatography to obtain 2-(3-methyl-4-nitrophenyl)-thioacetimidic acid 3,3,4,4,4-(pentafluoro)-2-oxobutyl ester (1.30 g).

¹H-NMR (CDCl₃, δ ppm): 2.55 (3H, s), 3.57 (2H, d), 3.90 (2H, d), 7.24-7.22 (2H, m), 7.91-7.89 (1H, m).

### SYNTHESIS EXAMPLE 31-3

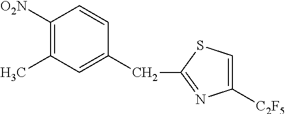

Trifluoroacetic anhydride (1.47 g) was added to a dichloromethane solution (10 mL) of 2-(3-methyl-4-nitrophenyl)thioacetoimidic acid 3,3,4,4,4-(pentafluoro)-2-oxobutyl ester (1.30 g) and triethylamine (0.71 g), and the mixture was stirred at room temperature for 20 minutes. After the reaction mixture was washed with water, the solvent was distilled off, and the resulting crude product was purified with

silica gel column chromatography to obtain 2-(3-methyl-4-nitrobenzyl)-4-(pentafluoroethyl)-1,3-thiazole (0.70 g).

¹H-NMR (CDCl₃, δ ppm): 2.63 (3H, s), 4.43 (2H, s), 7.30-7.28 (2H, m), 7.75 (1H, s), 7.98 (1H, d).

### SYNTHESIS EXAMPLE 31-4

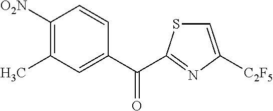

An acetonitrile solution (20 mL) of 2-(3-methyl-4-nitrobenzyl)-4-(pentafluoroethyl)-1,3-thiazole (0.80 g), cerium (IV) ammonium nitrate (0.62 g) and iodine (0.35 g) was heated to reflux for 5 days. After cooled to room temperature, the reaction mixture was diluted with ethyl acetate, washed sequentially with water, saturated aqueous solution of sodium thiosulfate and saturated brine, and dried over anhydrous magnesium sulfate. The solvent was distilled off, and the crude product was subjected to silica gel column chromatography (mixed solvent of n-hexane and ethyl acetate) to obtain (3-methyl-4-nitrophenyl)[4-(pentafluoroethyl)-1,3-thiazol-2-yl]methanone (0.35 g).

¹H-NMR (CDCl₃, δ ppm): 2.68 (3H, s), 8.06 (1H, d), 8.23 (1H, s), 8.47-8.54 (2H, m).

### SYNTHESIS EXAMPLE 31-5

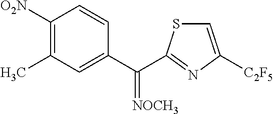

After a mixture of (3-methyl-4-nitrophenyl)[4-(pentafluoroethyl)-1,3-thiazole-2-yl]methanone (0.20 g), O-methylhydroxylammonium chloride (0.06 g), pyridine (5 mL), and ethanol (1 mL) was heated to reflux for 10 hours, the solvent was distilled off. The crude product was diluted with ethyl acetate, washed sequentially with water, 2N hydrochloric acid and saturated brine, and dried over anhydrous magnesium sulfate. The solvent was distilled off, and the crude product was subjected to silica gel column chromatography (mixed solvent of n-hexane and ethyl acetate) to obtain (3-methyl-4-nitrophenyol)[4-(pentafluoroethyl)-1,3-thiazol-2-yl]methanone O-methyloxime (0.21 g).

¹H-NMR (CDCl₃, δ ppm): 2.66 (3H, s), 4.32 (3H, s), 7.72-7.81 (2H, m), 8.03 (1H, d), 8.10 (1H, s).

### SYNTHESIS EXAMPLE 32-1

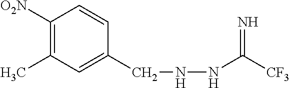

Trifluoroacetamidine (8.38 g) was added to a THF solution (150 mL) of (3-methyl-4-nitrobenzyl) hydrazine (13.55 g), and the mixture was stirred at room temperature for 20 hours. After the reaction was completed, the solvent has distilled off, and the crude product was purified with silica gel column chromatography (mixed solvent of n-hexane and ethyl acetate) to obtain 2,2,2-trifluoro-N′-(3-methyl-4-nitrobenzyl)ethanimide hydrazide (16.20 g).

¹H-NMR (CDCl₃, δ ppm): 2.61 (3H, s), 4.16-4.46 (5H, m), 7.26-7.40 (2H, m), 7.96 (1H, d).

### SYNTHESIS EXAMPLE 32-2

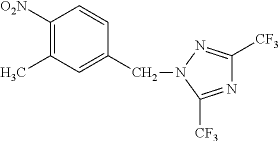

Trifluoro acetic anhydride (0.27 g) was added to a toluene solution (10 mL) of a 2,2,2-trifluoro-N′-(3-methyl-4-nitrobenzyl)ethanimide hydrazide (0.29 g), and the mixture was stirred at room temperature for 1 hour. After the solvent was distilled off, the resulting crude product was subjected to silica gel column chromatography (mixed solvent of n-hexane and ethyl acetate) to obtain 1-(3-methyl-4-nitrobenzyl)-3,5-bis(trifluoromethyl)-1H-1,2,4-triazole (0.21 g).

¹H-NMR (CDCl₃, δ ppm): 2.62 (3H, s), 5.56 (2H, s), 7.26-7.31 (2H, m), 7.99 (1H, d).

### SYNTHESIS EXAMPLE 32-3

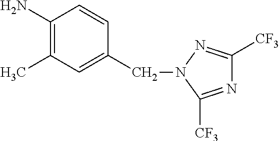

As similar to SYNTHESIS EXAMPLE 17-2, 4-{[3,5-bis(trifluoromethyl)-1H-1,2,4-triazol-1-yl]methyl}-2-methylaniline (0.60 g) was obtained from 1-(3-methyl-4-nitrobenzyl)-3,5-bis(trifluoromethyl)-1H-1,2,4-triazole (1.05 g), ammonium acetate (20.57 g), acetone (20 mL), water (60 mL) and 20% titanium trichloride aqueous solution (20.57 g).

¹H-NMR (CDCl₃, δ ppm): 2.15 (3H, s), 3.55-3.88 (2H, m), 5.37 (2H, s), 6.64 (1H, d), 7.03 (1H, d), 7.04 (1H, s).

### SYNTHESIS EXAMPLE 33-1

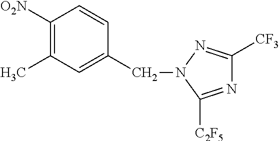

N-(3-dimethylaminopropyl)-N′-ethylcarbodiimide hydrochloride (13.50 g) was added to a dioxane solution (300 mL) of 2,2,2-trifluoro-N′-(3-methyl-4-nitrobenzyl)ethanimide hydrazide (16.20 g) and pentafluoropropionic acid (19.24 g), and the mixture was heated and stirred for 48 hours. After the reaction mixture was cooled to room temperature, water was added, and the mixture was extracted with ethyl acetate and dried over anhydrous magnesium sulfate. The solvent was distilled off, and the resulting crude product was purified with silica gel column chromatography (mixed solvent of n-hexane and ethyl acetate) to obtain 1-(3-methyl-4-nitrobenzyl)-5-(pentafluoroethyl)-3-(trifluoromethyl)-1H-1,2,4-triazole (23.2 g).

¹H-NMR (CDCl₃, δ ppm): Same as the first elution portion in SYNTHESIS EXAMPLE 8-1.

### SYNTHESIS EXAMPLE 33-2

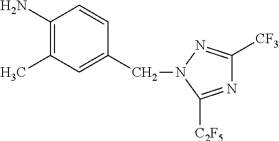

As similar to SYNTHESIS EXAMPLE 17-2, 2-methyl-4-{[5-(pentafluoroethyl)-3-(trifluoromethyl)-1H-1,2,4-triazol-1-yl]methyl}aniline (0.30 g) was obtained from 1-(3-methyl-4-nitrobenzyl)-5-(pentafluoroethyl)-3-(trifluoromethyl)-1H-1,2,4-triazole (0.34 g), ammonium acetate (6.48 g), acetone (12 mL), water (7 mL) and 20% titanium trichloride aqueous solution (5.8 g).

¹H-NMR (CDCl₃, δ ppm): 2.15 (3H, s), 3.34-3.93 (2H, m), 5.40 (2H, s), 6.64 (1H, d), 7.00-7.12 (2H, m).

### SYNTHESIS EXAMPLE 34-1

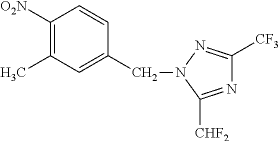

As similar to SYNTHESIS EXAMPLE 33-1, as pale yellow oily matter, 1-(3-methyl-4-nitrobenzyl)-5-(difluoromethyl)-3-(trifluoromethyl)-1H-1,2,4-triazole (3.70 g) was obtained from 2,2,2-trifluoro-N′-(3-methyl-4-nitrobenzyl)ethanimide hydrazide (3.20 g), difluoroacetic acid (2.11 g), and N-(3-dimethylaminopropyl)-N′-ethylcarbodiimide hydrochloride (2.53 g).

¹H-NMR (CDCl₃, δ ppm): 2.62 (3H, s), 5.58 (2H, s), 6.92 (1H, t), 7.27-7.35 (2H, m), 7.99 (1H, d).

### SYNTHESIS EXAMPLE 34-2

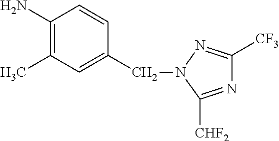

As similar to SYNTHESIS EXAMPLE 1-4, 4-4-{[5-(difluoromethyl)-3-(trifluoromethyl)-1H-1,2,4-triazol-1-yl]methyl}-2-methylaniline (2.41 g) was obtained from 1-(3-methyl-4-nitrobenzyl)-5-(difluoromethyl)-3-(trifluoromethyl)-1H-1,2,4-triazole (3.70 g), tin (II) chloride dihydrate (10.53 g), concentrated hydrochloric acid (11 mL) and ethanol (20 mL).

¹H-NMR (CDCl₃, δ ppm): 2.15 (3H, s), 3.53-3.85 (2H, m), 5.39 (2H, s), 6.64 (1H, d), 6.81 (1H, t), 7.01-7.11 (2H, m).

### SYNTHESIS EXAMPLE 35-1

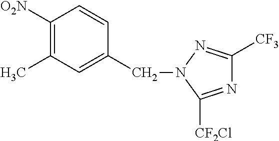

As similar to SYNTHESIS EXAMPLE 32-2, 1-(3-methyl-4-nitrobenzyl)-5-[chloro(difluoro)methyl]-3-(trifluoromethyl)-1H-1,2,4-triazole (5.57 g) was obtained from 2,2,2-trifluoro-W-(3-methyl-4-nitrobenzyl)ethanimide hydrazide (5.16 g) and chlorodifluoroacetic anhydride (5.90 g).

¹H-NMR (CDCl₃, δ ppm): 2.60 (3H, s), 5.59 (2H, s), 7.25-7.31 (2H, m), 7.99 (1H, d).

### SYNTHESIS EXAMPLE 35-2

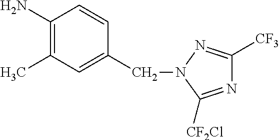

As similar to SYNTHESIS EXAMPLE 1-4, 4-({5-[chloro(difluoro) methyl]-3-(trifluoromethyl)-1H-1,2,4-triazol-1-yl}methyl)-2-methylaniline (4.00 g) was obtained from 1-(3-methyl-4-nitrobenzyl)-5-[chloro(difluoro)methyl]-3-(trifluoromethyl)-1H-1,2,4-triazole (4.50 g), tin (II) chloride dihydrate (13.7 g), concentrated hydrochloric acid (14 mL) and ethanol (25 mL).

¹H-NMR (CDCl₃, δ ppm): 2.16 (3H, s), 3.50-3.92 (2H, m), 5.40 (2H, s), 6.63 (1H, d), 6.99-7.09 (2H, m)

### SYNTHESIS EXAMPLE 36-1

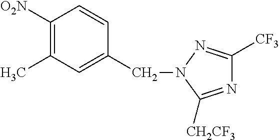

As similar to SYNTHESIS EXAMPLE 33-1, 1-(3-methyl-4-nitrobenzyl)-5-(2,2,2-trifluoroethyl)-3-(trifluoromethyl)-1H-1,2,4-triazole (1.00 g) was obtained from 2,2,2-trifluoro-N′-(3-methyl-4-nitrobenzyl)ethanimide hydrazide (1.00 g), 3,3,3-trifluoropropionic acid (0.93 g) and N-(3-dimethylaminopropyl)-N′-ethyl carbodiimide hydrochloric acid salt (0.83 g).

¹H-NMR (CDCl₃, δ ppm): 2.62 (3H, s), 3.69 (2H, q), 5.47 (2H, s), 7.13-7.22 (2H, m), 8.00 (1H, d).

### SYNTHESIS EXAMPLE 36-2

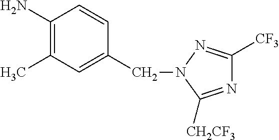

As similar to SYNTHESIS EXAMPLE 15-2, 2-methyl-4-{[5-(2,2,2-trifluoroethyl)-3-(trifluoromethyl)-1H-1,2,4-triazol-1-yl]methyl}aniline (0.60 g) was obtained from 1-(3-methyl-4-nitrobenzyl)-5-(2,2,2-trifluoroethyl)-3-(trifluoromethyl)-1H-1,2,4-triazole (1.00 g), ammonium acetate (18.8 g), acetone (30 mL), water (90 mL) and 20% titanium trichloride aqueous solution (18.8 g).

¹H-NMR (CDCl₃, δ ppm): 2.14 (3H, s), 3.58 (2H, q), 3.87-4.35 (2H, m), 5.31 (2H, s), 6.65 (1H, d), 6.86-6.95 (2H, m).

### SYNTHESIS EXAMPLE 37-1

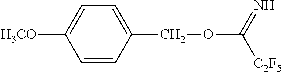

Pentafluoro propionic acid amide (20 g) was dissolved in a mixed solvent of DMSO (44 mL) and dichloromethane (300 mL), and the mixture was cooled to −78° C. in a dry ice-acetone bath. Subsequently, oxalyl chloride (18.7 g) was slowly added dropwise, and after stirred at −78° C. for 10 minutes, triethylamine (51 mL) was added dropwise at the same temperature. After stirred for 30 minutes, DBU (39.2 g) was added, and then a dichloromethane solution of 4-methoxybenzyl alcohol (17.8 g) was added dropwise. After 15 minutes, the dry ice-acetone bath was removed, and the temperature was gradually returned to room temperature with stirring for 10 hours. Water and ethyl acetate were added to the reaction mixture, and the organic phase was separated, and dried over anhydrous magnesium sulfate. The solvent was distilled off, and the resulting crude product was purified with silica gel column chromatography (mixed solvent of n-hexane and ethyl acetate) to obtain 2,2,3,3,3-pentafluoropropionimidic acid 4-methoxybenzyl ester (7.15 g).

¹H-NMR (CDCl₃, δ ppm): 3.81 (3H, s), 5.26 (2H, s), 6.91 (2H, d), 7.32 (2H, d), 8.37-8.47 (1H, m).

### SYNTHESIS EXAMPLE 37-2

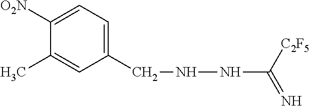

2,2,3,3,3-pentafluoropropionimidic acid 4-methoxybenzyl ester (7.15 g) was added to a THF solution (20 mL) of (3-methyl-4-nitrobenzyl)hydrazine (4.58 g), and the mixture was stirred at room temperature for 20 hours. After the reaction was completed, the solvent was distilled off, and the resulting crude product was purified with silica gel column chromatography (mixed solvent of n-hexane and ethyl acetate) to obtain 2,2,3,3,3-pentafluoro-N′-(3-methyl-4-nitrobenzyl)propanimide hydrazide (8.70 g).

¹H-NMR (CDCl₃, δ ppm): same as the SYNTHESIS EXAMPLE 19-1.

### SYNTHESIS EXAMPLE 37-3

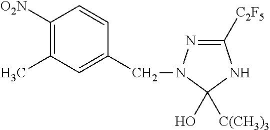

To a mixture of 2,2,3,3,3-pentafluoro-N′-(3-methyl-4-nitrobenzyl)propanimide hydrazide (0.34 g), 10% aqueous solution of sodium hydrogen carbonate (2.5 mL) and toluene (5 mL), pivaloyl chloride (0.15 g) was added, and the mixture was stirred at room temperature for 2 hours. The organic phase was separated and dried over anhydrous sodium sulfate. The solvent was distilled off, and the crude product was subjected to silica gel column chromatography (mixed solvent of n-hexane and ethyl acetate) to obtain 5-t-butyl-1-(3-methyl-4-nitrobenzyl)-3-(pentafluoroethyl)-4,5-dihydro-1H-1,2,4-triazol-5-ol (0.30 g) as colorless solid.

Melting point: 125-128° C.

¹H-NMR (CDCl₃, δ ppm): 1.24 (9H, s), 2.57 (3H, s), 4.67 (2H, s), 5.82-5.96 (2H, m), 7.20-7.29 (2H, m), 7.93 (1H, d).

### SYNTHESIS EXAMPLE 37-4

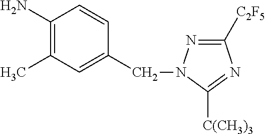

To a mixture of tin (II) chloride dihydrate (0.69 g), concentrated hydrochloric acid (0.7 mL) and ethanol (1 mL), 5-t-butyl-1-(3-methyl-4-nitrobenzyl)-3-(pentafluoroethyl)-4,5-dihydro-1H-1,2,4-triazol-5-ol (0.25 g) was added under ice cooling, and the mixture was heated and stirred at 70° C. for 1 hour. After cooled to room temperature, the reaction mixture was poured into ice and adjusted to pH 11 or above with sodium hydroxide, and then extracted with t-butylmethylether. The organic phase was washed with water and dried over anhydrous sodium sulfate, and the solvent was distilled off to obtain 4-{[5-t-butyl-3-(pentafluoroethyl)-1H-1,2,4-triazole-1-yl]methyl}-2-methylaniline (0.10 g).

¹H-NMR (CDCl₃, δ ppm): 1.40 (9H, s), 2.12 (3H, s), 3.53-3.71 (2H, m), 5.42 (2H, s), 6.61 (1H, d), 6.76 (1H, d), 6.81 (1H, s).

### SYNTHESIS EXAMPLE 38-1

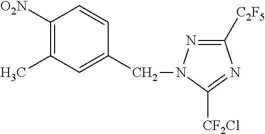

As similar to SYNTHESIS EXAMPLE 32-2, from 2,2,3,3,3-pentafluoro-N′-(3-methyl-4-nitrobenzyl)propanimide hydrazide (1.72 g) and chloro (difluoro) acetic anhydride (1.58 g), 1-(3-methyl-4-nitrobenzyl)-5-[chloro(difluoro)-methyl]-3-(pentafluoroethyl)-1H-1,2,4-triazole (2.03 g) was obtained.

¹H-NMR (CDCl₃, δ ppm): 2.60 (3H, s), 5.60 (2H, s), 7.20-7.34 (2H, m), 7.99 (1H, d).

### SYNTHESIS EXAMPLE 38-2

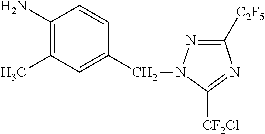

As similar to SYNTHESIS EXAMPLE 1-4, from 1-(3-methyl-4-nitrobenzyl)-5-[chloro(difluoro)methyl]-3-(pentafluoroethyl)-1H-1,2,4-triazole (2.20 g), tin (II) chloride dihydrate (5.90 g), concentrated hydrochloric acid (6 mL) and ethanol (10 mL), 4-({5-[chloro(difluoro)methyl]-3-pentafluoroethyl}-1H-1,2,4-triazol-1-yl)methyl)-2-methylaniline (1.80 g) was obtained.

¹H-NMR (CDCl₃, δ ppm): 2.15 (3H, s), 3.41-3.95 (2H, m), 5.42 (2H, s), 6.63 (1H, d), 6.99-7.06 (2H, m).

### SYNTHESIS EXAMPLE 39-1

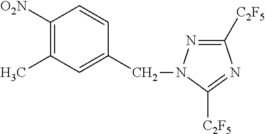

As similar to SYNTHESIS EXAMPLE 32-2, from 2,2,3,3,3-pentafluoro-N′-(3-methyl-4-nitrobenzyl)propanimide hydrazide (20.60 g) and pentafluoropropionic anhydride (22.32 g), 1-(3-methyl-4-nitrobenzyl)-3,5-bis(pentafluoroethyl)-1H-1,2,4-triazole (23.61 g) was obtained.

¹H-NMR (CDCl₃, δ ppm): 2.61 (3H, s), 5.62 (2H, s), 7.23-7.31 (2H, m), 7.99 (1H, d).

### SYNTHESIS EXAMPLE 39-2

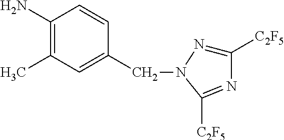

As similar to SYNTHESIS EXAMPLE 1-4, from 1-(3-methyl-4-nitrobenzyl)-3,5-bis(pentafluoroethyl)-1H-1,2,4-triazole (22.71 g), tin (II) chloride dihydride (56.41 g), concentrated hydrochloric acid (58 mL) and ethanol (80 mL), 1-(4-amino-3-methylphenyl)-2-[3,5-bis(trifluoromethyl)-1H-pyrazol-1-yl]ethanone (17.16 g) was obtained.

¹H-NMR (CDCl₃, δ ppm): 2.15 (3H, s), 3.50-3.90 (2H, m), 5.42 (2H, s), 6.63 (1H, d), 7.02 (1H, d), 7.04 (1H, s).

### SYNTHESIS EXAMPLE 40-1

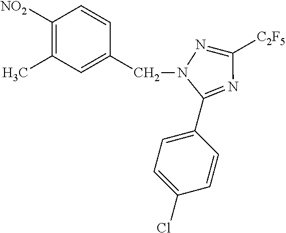

To a mixture of 2,2,3,3,3-pentafluoro-N′-(3-methyl-4-nitrobenzyl)propaneimide hydrazide (1.03 g), 5% aqueous solution of sodium hydrogen carbonate (15 mL) and toluene (25 mL), 4-chlorobenzoylchloride (0.63 g) was added dropwise under the ice cooling, and the mixture was stirred at room temperature for 3 hours. The precipitate was collected by filtration, washed with water, and air-dried to obtain 5-(4-chlorophenyl)-1-(3-methyl-4-nitrobenzyl)-3-(pentafluoroethyl)-4,5-dihydro-1H-1,2,4-triazol-5-ol (1.00 g) as colorless solid.

Melting point: 90-92° C.

Subsequently, 5-(4-chlorophenyl)-1-(3-methyl-4-nitrobenzyl)-3-(pentafluoroethyl)-4,5-dihydro-1H-1,2,4-triazol-5-ol (0.93 g) and trifluoroacetic acid (0.31 mL) were heated to reflux in toluene (20 mL) for 2 hours. After left to cool to room temperature, the solvent was distilled off, and the crude product was subjected to silica gel column chromatography (mixed solvent of n-hexane and ethyl acetate) to obtain 5-(4-chlorophenyl)-1-(3-methyl-4-nitrobenzyl)-3-(pentafluoroethyl)-1H-1,2,4-triazole (0.85 g) as pale yellow oily matter.

¹H-NMR (CDCl₃, δ ppm): 2.59 (3H, s), 5.51 (2H, s), 7.07-7.16 (2H, m), 7.50 (4H, bs), 7.99 (1H, d).

### SYNTHESIS EXAMPLE 40-2

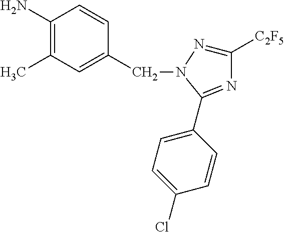

As similar to SYNTHESIS EXAMPLE 17-2, from 5-(4-chlorophenyl)-1-(3-methyl-4-nitrobenzyl)-3-(pentafluoroethyl)-1H-1,2,4-triazole (0.50 g), ammonium acetate (8.62 g), acetone (25 mL), water (25 mL) and 20% titanium trichloride aqueous solution (7.77 g), 4-{[5-(4-chlorophenyl)-3-(pentafluoroethyl)-1H-1,2,4-triazol-1-yl]methyl}-2-methylaniline (0.46 g) was obtained.

¹H-NMR (CDCl₃, δ ppm): 2.12 (3H, s), 3.60-3.88 (2H, m), 5.32 (2H, s), 6.62 (1H, d), 6.80 (1H, d), 7.47 (2H, d), 7.55 (2H, d).

### SYNTHESIS EXAMPLE 41-1

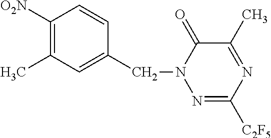

A toluene solution (50 mL) of 2,2,3,3-pentafluoro-N′-(3-methyl-4-nitrobenzyl)propanimide hydrazide (1.63 g), methyl pyruvate (0.61 g) and p-toluene sulfonic acid monohydrate (0.05 g) was heated to reflux for 3 hours while dehydrated. After cooled to room temperature, the solvent was distilled off, and the crude product was subjected to silica gel column chromatography (mixed solvent of n-hexane and ethyl acetate) to obtain 5-methyl-1-(3-methyl-4-nitrobenzyl)-3-(pentafluoroethyl)-1,2,4-triazin-6(1H)-one (0.28 g).

¹H-NMR (CDCl₃, δ ppm): 2.59 (3H, s), 2.60 (3H, s), 5.27 (2H, s), 7.39-7.47 (2H, m), 7.95 (1H, d).

### SYNTHESIS EXAMPLE 41-2

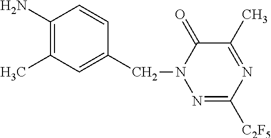

As similar to SYNTHESIS EXAMPLE 1-4, from 5-methyl-1-(3-methyl-4-nitrobenzyl)-3-(pentafluoroethyl)-1,2,4-triazin-6(1H)-one (0.26 g), tin (II) chloride dihydrate (0.78 g), concentrated hydrochloric acid (0.8 mL) and ethanol (1.2 mL), 1-(4-amino-3-methylbenzyl)-5-methyl-3-(pentafluoroethyl)-1,2,4-triazin-6(1H)-one (0.21 g) was obtained.

¹H-NMR (CDCl₃, δ ppm): 2.13 (3H, s), 2.14 (3H, s), 3.58 (2H, ns), 5.12 (2H, s), 6.61 (1H, d), 7.00-7.11 (2H, m).

### SYNTHESIS EXAMPLE 42-1

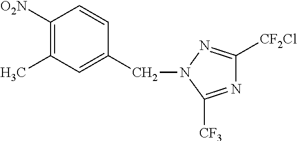

To a THF solution (30 mL) of (3-methyl-4-nitrobenzyl)hydrazine (3.26 g), chlorodifluoroacetamidine (2.57 g) was added, and the mixture was stirred at room temperature for 2 hours. Subsequently, trifluoroacetic anhydride (6.31 g) was added and the mixture was stirred at room temperature for 2 hours. The reaction mixture was diluted with t-butylmethylether, washed with saturated aqueous solution of sodium hydrogen carbonate and dried over anhydrous sodium sulfate. After the solvent was distilled off, the resulting crude product was purified with silica gel column chromatography (mixed solvent of n-hexane and ethyl acetate) to obtain 3-[chloro(difluoro)methyl]-1-(3-methyl-4-nitrobenzyl)-5-(trifluoromethyl)-1H-1,2,4-triazole (0.50 g).

¹H-NMR (CDCl₃, δ ppm): 2.62 (3H, s), 5.58 (2H, s), 7.26-7.33 (2H, m), 7.99 (1H, d).

### SYNTHESIS EXAMPLE 42-2

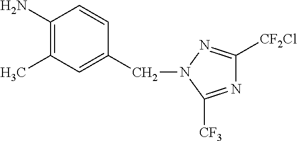

As similar to SYNTHESIS EXAMPLE 1-4, from 3-[chloro(difluoro)methyl]-1-(3-methyl-4-nitrobenzyl)-5-(trifluoromethyl)-1H-1,2,4-triazole (0.50 g), tin (II) chloride (1.52 g), concentrated hydrochloric acid (1.6 mL) and ethanol (2.5 mL), 4-({3-[chloro(difluoro)methyl]-5-(trifluoromethyl)-1H-1,2,4-triazol-1-yl}methyl)-2-methylaniline (0.35 g) was obtained.

¹H-NMR (CDCl₃, δ ppm): 2.15 (3H, s), 3.71 (2H, bs), 5.37 (2H, s), 6.64 (1H, d), 7.01-7.07 (2H, m)

### SYNTHESIS EXAMPLE 43-1

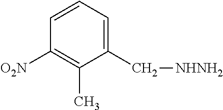

To an ethanol solution (100 mL) of hydrazine hydrate (6.74 g), an ethanol solution (20 mL) of 2-methyl-3-nitrobenzylchloride (5.00 g) was added for 30 minutes while heating to reflux, and the mixture was further heated to reflux for 1 hour. After left to cool at room temperature, the solvent was distilled off, and the residue was dissolved in ethyl acetate, washed sequentially with water and saturated brine, and dried over anhydrous sodium sulfate. The solvent was distilled off to obtain (2-methyl-3-nitrobenzyl)hydrazine (3.72 g).

¹H-NMR (CDCl₃, δ ppm): 2.48 (3H, s), 2.94-3.21 (3H, m), 4.01 (2H, s), 7.31 (1H, dd), 7.55 (1H, d), 7.70 (1H, d).

### SYNTHESIS EXAMPLE 43-2

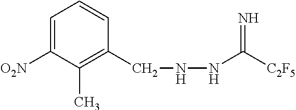

As similar to SYNTHESIS EXAMPLE 32-1, from (2-methyl-3-nitrobenzyl)hydrazine (3.72 g) and pentafluoropropanamidine (3.33 g), 2,2,3,3,3-pentafluoro-N′-(2-methyl-3-nitrobenzyl)propanimide hydrazide (6.20 g) was obtained.

¹H-NMR (CDCl₃, δ ppm): 2.49 (3H, s), 4.22-4.50 (3H, m), 4.38 (2H, s), 7.30 (1H, dd), 7.52 (1H, d), 7.70 (1H, d)

### SYNTHESIS EXAMPLE 43-3

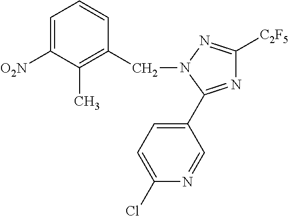

As similar to SYNTHESIS EXAMPLE 33-1, from 2,2,3,3,3-pentafluoro-N′-(2-methyl-3-nitrobenzyl)propanimide hydrazide (0.78 g), 6-chloro nicotinic acid (0.42 g) and N-(3-dimethylaminopropyl)-N′-ethylcarbodiimide hydrochloride (0.69 g), 2-chloro-5-[1-(2-methyl-3-nitrobenzyl)-3-(pentafluoroethyl)-1H-1,2,4-triazol-5-yl]pyridine (0.49 g) was obtained.

¹H-NMR (CDCl₃, δ ppm): 2.39 (3H, s), 5.59 (2H, s), 6.94 (1H, d), 7.35 (1H, dd), 7.51 (1H, d), 7.79 (1H, d), 7.93 (1H, dd), 8.55 (1H, d).

### SYNTHESIS EXAMPLE 43-4

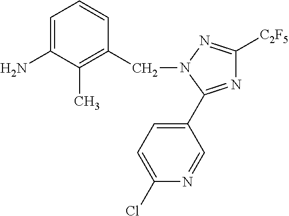

As similar to SYNTHESIS EXAMPLE 1-4, from 2-chloro-5-[1-(2-methyl-3-nitrobenzyl)-3-(pentafluoroethyl)-1H-1,2,4-triazol-5-yl]pyridine (0.43 g), tin (II) chloride dihydride (1.08 g), concentrated hydrochloric acid (1.2 mL) and ethanol (2 mL), 3-{[5-(6-chloropyridin-3-yl)-3-(pentafluoroethyl)-1H-1,2,4-triazol-1-yl]methyl}-2-methylaniline (0.34 g) was obtained.

¹H-NMR (CDCl₃, δ ppm): 2.04 (3H, s), 3.71 (2H, bs), 5.51 (2H, s), 6.67 (1H, d), 6.95 (1H, dd), 7.42 (1H, d), 7.88 (1H, dd), 8.54 (1H, d).

### SYNTHESIS EXAMPLE 44-1

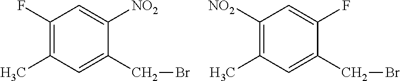

A mixture of 1-fluoro-2,4-dimethyl-5-nitrobenzene (10.00 g), N-bromosuccinimide (18.94 g) and azodiisobutyronitrile (0.97 g) was heated to reflux in carbon tetrachloride (140 mL) under lighting for 6 hours. After the reaction was completed, an insoluble matter was filtered off, and the filtrate was concentrated to obtain a mixture (14.10 g) of 1-(bromomethyl)-2-fluoro-5-methyl-4-nitrobenzene and 1-(bromomethyl)-4-fluoro-5-methyl-2-nitrobenzene at an approximate ratio of 1:1 as oily matter, which was subjected to the next reaction without purification.

### SYNTHESIS EXAMPLE 44-2

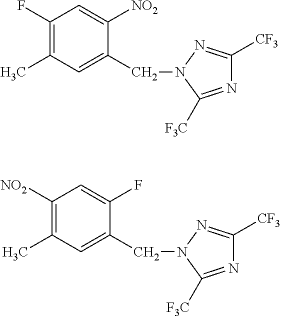

A mixture of the crude mixture (14.00 g) of 1-(bromomethyl)-2-fluoro-5-methyl-4-nitrobenzene and 1-(bromomethyl)-4-fluoro-5-methyl-2-nitrobenzene obtained in SYNTHESIS EXAMPLE 44-1, 3,5-bis(trifluoromethyl)-1H-1,2,4-triazole (6.94 g) and potassium carbonate (9.36 g) were heated and the mixture was stirred in DMF (120 mL) at 60° C. for 1 hour. After left to cool to room temperature, an insoluble matter was filtered off. The filtrate was concentrated, and the crude product was purified with silica gel column chromatography (mixed solvent of cyclohexane and ethyl acetate) to obtain, as the first elution portion, 1-(4-fluoro-5-methyl-2-nitrobenzyl)-3,5-bis(trifluoromethyl)-1H-1,2,4-triazole (2.80 g);

¹H-NMR (DMSO-d₆, δ ppm): 2.35 (3H, s), 5.97 (2H, s), 7.47 (1H, d), 8.03 (1H, d);

and, as the second elution portion, 1-(2-fluoro-5-methyl-4-nitrobenzyl)-3,5-bis(trifluoromethyl)-1H-1,2,4-triazole (2.40 g).

¹H-NMR (DMSO-d₆, δ ppm): 2.50 (3H, s), 5.81 (2H, s), 7.57 (1H, d), 7.94 (1H, d).

### SYNTHESIS EXAMPLE 44-3

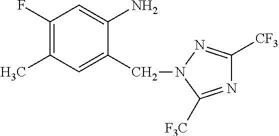

As similar to SYNTHESIS EXAMPLE 1-4, from 1-(4-fluoro-5-methyl-2-nitrobenzyl)-3,5-bis(trifluoromethyl)-1H-1,2,4-triazole (2.70 g), tin (II) chloride dihydrate (7.78 g), concentrated hydrochloric acid (15 mL) and ethanol (25 mL), 2-{[3,5-bis(trifluoromethyl)-1H-1,2,4-triazol-1-yl]methyl}-5-fluoro-4-methylaniline (1.90 g) was obtained.

¹H-NMR (DMSO-d₆, δ ppm): 2.02 (3H, d), 2.90-3.80 (2H, m), 5.48 (2H, s), 6.50 (1H, d), 6.77 (1H, d).

### SYNTHESIS EXAMPLE 44-4

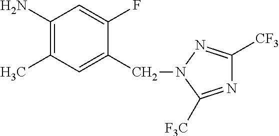

As similar to SYNTHESIS EXAMPLE 1-4, from 1-(2-fluoro-5-methyl-4-nitrobenzyl)-3,5-bis(trifluoromethyl)-1H-1,2,4-triazole (2.10 g), tin (II) chloride dihydrate (5.51 g), concentrated hydrochloric acid (10 mL) and ethanol (20 mL), 4-{[3,5-bis(trifluoromethyl)-1H-1,2,4-triazol-1-yl]methyl}-5-fluoro-2-methylaniline (1.20 g) was obtained.

¹H-NMR (DMSO-d₆, δ ppm): 2.00 (3H, d), 3.30-4.00 (2H, m), 5.50 (2H, s), 6.41 (1H, d), 6.90-7.0 (1H, m).

### SYNTHESIS EXAMPLE 45-1

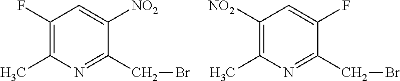

As similar to SYNTHESIS EXAMPLE 44-1, 3-fluoro-2,6-dimethyl-5-nitro pyridine (3.00 g), N-bromosuccinimide (4.08 g) and azodiisobutyronitrile (0.29 g) were heated to reflux in carbon tetrachloride (40 mL) under lighting for 5 hours. After the reaction was completed, an insoluble matter was filtered, and the filtrate was concentrated. The crude product was subjected to silica gel column chromatography (mixed solvent of cyclohexane and ethyl acetate) to obtain a mixture (0.55 g) of 2-(bromomethyl)-5-fluoro-6-methyl-3-nitropyridine and 2-(bromomethyl)-3-fluoro-6-methyl-5-nitropyridine at an approximate ratio of 1:1 as oily matter, which was subjected to the next reaction without further purification.

¹H-NMR (DMSO-d₆, δ ppm): 2.54 and 2.71 (3H, s), 4.72 and 4.87 (2H, s), 8.43 and 8.47 (1H, d).

### SYNTHESIS EXAMPLE 45-2

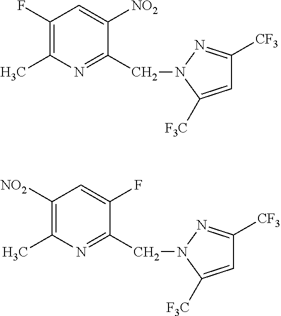

As similar to SYNTHESIS EXAMPLE 44-2, from the crude mixture (0.50 g) of 2-(bromomethyl)-5-fluoro-6-methyl-3-nitropyridine and 2-(bromomethyl)-3-fluoro-6-methyl-5-nitropyridine at an approximate ratio of 1:1 obtained in SYNTHESIS EXAMPLE 45-1, 3,5-bis(trifluoromethyl)-1H-pyrazole (0.20 g), potassium carbonate (0.32 g) and DMF (3 mL), 2-{[3,5-bis(trifluoromethyl)-1H-pyrazol-1-yl]methyl}-5-fluoro-6-methyl-3-nitropyridine and 2-{[3,5-bis(trifluoromethyl)-1H-pyrazol-1-yl]methyl}-3-fluoro-6-methyl-5-nitropyridine (0.72 g) were obtained, which were subjected to the next reaction without further purification.

### SYNTHESIS EXAMPLE 45-3

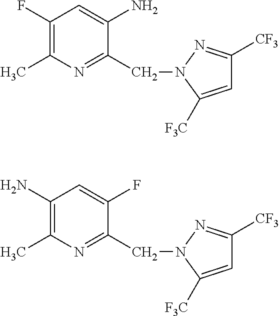

As similar to SYNTHESIS EXAMPLE 1-4, the crude product obtained from the mixture (0.72 g) of 2-{[3,5-bis(trifluoromethyl)-1H-pyrazol-1-yl]methyl}-5-fluoro-6-methyl-3-nitropyridine and 2-{[3,5-bis(trifluoromethyl)-1H-pyrazol-1-yl]methyl}-3-fluoro-6-methyl-5-nitropyridine, tin (II) chloride dihydrate (2.04 g), concentrated hydrochloric acid (10 mL) and ethanol (10 mL) was subjected to silica gel column chromatography (mixed solvent of cyclohexane and ethyl acetate) to obtain, as the first elution portion, 2-{[3,5-bis(trifluoromethyl)-1H-pyrazol-1-yl]methyl}-5-fluoro-6-methylpyridin-3-amine (0.076 g);

¹H-NMR (DMSO-d₆, δ ppm): 2.09 (3H, s), 5.25-5.50 (2H, m), 5.51 (2H, s), 6.86 (1H, d), 7.44 (1H, s);

and, as the second elution portion, 6-{[3,5-bis(trifluoromethyl)-1H-pyrazol-1-yl]methyl}-5-fluoro-2-methylpyridin-3-amine (0.055 g).

¹H-NMR (DMSO-d₆, δ ppm): 2.16 (3H, s), 5.28-5.40 (2H, m), 5.49 (2H, s), 6.76 (1H, d), 7.42 (1H, s).

### SYNTHESIS EXAMPLE 46-1

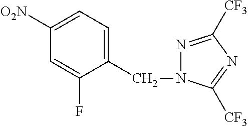

As similar to SYNTHESIS EXAMPLE 1-3, from 1-(bromomethyl)-2-fluoro-4-nitrobenzene (4.00 g), 3,5-bis(trifluoromethyl)-1H-1,2,4-triazole (3.65 g) and potassium carbonate (3.40 g), 1-(2-fluoro-4-nitrobenzyl)-3,5-bis(trifluoromethyl)-1H-1,2,4-triazole (3.60 g) was obtained.

¹H-NMR (CDCl₃, δ ppm): 5.69 (2H, s), 7.46 (1H, t), 8.02-8.12 (2H, m).

### SYNTHESIS EXAMPLE 46-2

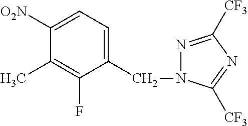

To a THF solution (5 mL) of 1-(2-fluoro-4-nitrobenzyl)-3,5-bis(trifluoromethyl)-1H-1,2,4-triazole (1.34 g), a THF solution (1.8 mL) of 3M methyl magnesium chloride was added at −50° C. or below, and the mixture was stirred at −70° C. for 10 minutes. Subsequently, 2,3-dichloro-5,6-dicyano-p-benzoquinone (1.27 g) was added thereto, and the dry ice bath was removed to return to room temperature gradually with stirring for 1 hour. Water was added to the reaction mixture, which was then extracted with ethyl acetate. The organic phase was washed with water and dried over anhydrous magnesium sulfate. The solvent was distilled off, and the resulting crude product was subjected to silica gel column chromatography (mixed solvent of n-hexane and ethyl acetate) to obtain 1-(2-fluoro-3-methyl-4-nitrobenzyl)-3,5-bis(trifluoromethyl)-1H-1,2,4-triazole (0.69 g).

¹H-NMR (CDCl₃, δ ppm): 2.53 (2.6H, d), 2.59 (0.4H, s), 5.63 (0.3H, s), 5.66 (1.7H, s), 7.19-7.26 (1H, m), 7.77-7.84 (1H, m).

### SYNTHESIS EXAMPLE 46-3

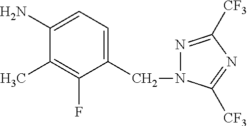

As similar to SYNTHESIS EXAMPLE 1-4, from 1-(2-fluoro-3-methyl-4-nitrobenzyl)-3,5-bis(trifluoromethyl)-1H-1,2,4-triazole (0.66 g), tin (II) chloride dihydrate (1.20 g), concentrated hydrochloric acid (3 mL) and methanol (9 mL), the objective compound 4-{[3,5-bis(trifluoromethyl)-1H-1,2,4-triazol-1-yl]methyl}-3-fluoro-2-methylaniline (0.56 g) was obtained.

¹H-NMR (CDCl₃, δ ppm): 2.06 (3H, d), 3.84 (2H, bs), 5.47 (2H, s), 6.45 (1H, d), 6.95 (1H, t).

### SYNTHESIS EXAMPLE 47

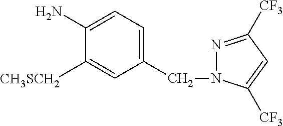

To a dichloromethane solution (6 mL) of 4-{[3,5-bis(trifluoromethyl)-1H-pyrazol-1-yl]methyl}aniline (0.80 g) and dimethylsulfide (0.23 g), N-chlorosuccinimide (0.48 g) was added at 15° C., and the mixture was stirred for 10 minutes. Subsequently, triethylamine (0.5 mL) was added, and the mixture was heated to reflux for 5 hours. After cooled to room temperature, the reaction mixture was washed with 10% sodium hydroxide aqueous solution and dried over anhydrous sodium sulfate. The solvent was distilled off, and the crude product was purified with silica gel column chromatography (mixed solvent of n-hexane and ethyl acetate) to obtain the objective compound 4-{[3,5-bis(trifluoromethyl)-1H-pyrazol-1-yl]methyl}-2-[(methylthio)methyl]aniline (0.64 g).

¹H-NMR (CDCl₃, δ ppm): 1.93 (3H, s), 3.64 (2H, s), 4.14 (2H, bs), 5.33 (2H, s), 6.65 (1H, d), 6.86 (1H, s), 6.98 (1H, d), 7.05 (1H, dd).

### SYNTHESIS EXAMPLE 48-1

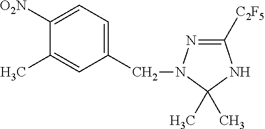

To 2,2,3,3,3-pentafluoro-N′-(3-methyl-4-nitrobenzyl)propanimide hydrazide (2.61 g), p-toluenesulfonic acid monohydrate (0.08 g) was added, and heated to reflux in acetone (60 mL) for 4 hours with dehydrating. After cooled to room temperature, the crude crystal obtained after the solvent was distilled off was washed with a mixed solvent of t-butylmethylether/petroleum ether to obtain 5,5-dimethyl-1-(3-methyl-4-nitrobenzyl)-3-(pentafluoroethyl)-5-(trifluoromethyl)-4,5-dihydro-1H-1,2,4-triazole (2.52 g).

¹H-NMR (CDCl₃, δ ppm): 1.43 (6H, s), 2.60 (3H, s), 4.14 (2H, s), 4.31 (1H, bs), 7.35-7.41 (2H, m), 7.96 (1H, d).

### SYNTHESIS EXAMPLE 48-2

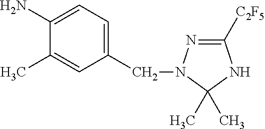

To an ethanol solution (10 mL) of 5,5-dimethyl-1-(3-methyl-4-nitrobenzyl)-3-(pentafluoroethyl)-5-(trifluoromethyl)-4,5-dihydro-1H-1,2,4-triazole (0.37 g), 5% (w/w) palladium-carbon (0.05 g) was added, and the mixture was stirred under hydrogen atmosphere at room temperature for 2 hours. After the reaction was completed, the palladium catalyst was filtered off with Celite. The filtrate was distilled off under reduced pressure to obtain 4-{[5,5-dimethyl-3-(pentafluoroethyl)-4,5-dihydro-1H-1,2,4-triazol-1-yl]methyl}-2-methylaniline (0.34 g).

¹H-NMR (CDCl₃, δ ppm): 1.33 (6H, s), 2.16 (3H, s), 4.07 (2H, s), 6.63 (1H, d), 7.05 (1H, d), 7.09 (1H, s).

### SYNTHESIS EXAMPLE 49-1

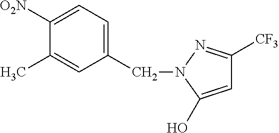

To a toluene solution (80 mL) of (3-methyl-4-nitrobenzyl)-hydrazine (7.0 g), ethyl 4,4,4-trifluoroacetoacetate (7.11 g) and p-toluenesulfonic acid monohydrate (0.37 g) were added, and the mixture was heated to reflux for 8 hours while dehydrated. After cooled to room temperature, the precipitated crystal was collected by filtration. The crystal was washed with mixed solvent of t-butylmethylether/hexane to obtain 1-(3-methyl-4-nitrobenzyl)-3-(trifluoromethyl)-1H-pyrazol-5-ol (6.99 g).

¹H-NMR (DMSO-d₆, δ ppm): 2.50 (3H, s), 5.25 (3H, s), 5.81 (1H, s), 7.18 (1H, d), 7.31 (1H, s), 7.99 (1H, d), 12.04 (1H, s).

### SYNTHESIS EXAMPLE 49-2

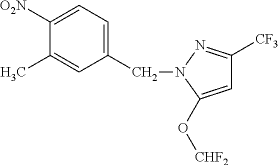

1-(3-methyl-4-nitrobenzyl)-3-(trifluoromethyl)-1H-pyrazole-5-ol (0.90 g) was dissolved in DMF (10 mL), and the mixture was placed into a pressure-resistant vessel made of glass with potassium carbonate (0.62 g). After chlorodifluoromethane (0.70 g) was added to the vessel, the vessel was tightly sealed, and the mixture was stirred at 90° C. for seven hours. After cooled to room temperature, the reaction mixture was poured into water and extracted with ethyl acetate. The organic phase was washed with water and dried over anhydrous magnesium sulfate. The solvent was distilled off, and the crude product was purified with silica gel column chromatography (mixed solvent of n-hexane and ethyl acetate) to obtain 1-(3-methyl-4-nitrobenzyl)-5-(difluoromethoxy)-3-(trifluoromethyl)-1H-pyrazole (0.67 g).

¹H-NMR (CDCl₃, δ ppm): 2.59 (3H, s), 5.29 (2H, s), 6.24 (1H, s), 6.53 (1H, t), 7.16-7.20 (2H, m), 7.96 (1H, d).

### SYNTHESIS EXAMPLE 49-3

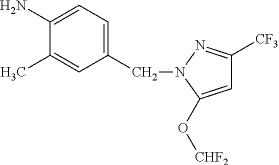

As similar to SYNTHESIS EXAMPLE 17-2, from 1-(3-methyl-4-nitrobenzyl)-5-(difluoromethoxy)-3-(trifluoromethyl)-1H-pyrazole (0.59 g), ammonium acetate (12.8 g), acetone (25 mL), water (13 mL) and 20% titanium trichloride aqueous solution (11.6 g), 4-{[5-(difluoromethoxy)-3-(trifluoromethyl)-1H-pyrazol-1-yl]methyl}-2-methylaniline (0.50 g) was obtained.

¹H-NMR (CDCl₃, δ ppm): 2.13 (3H, s), 3.65 (2H, bs), 5.12 (2H, s), 6.15-6.64 (3H, m), 6.95-7.06 (2H, m).

### SYNTHESIS EXAMPLE 50-1

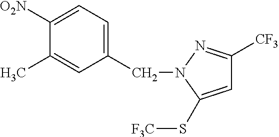

A mixture of 1-(3-methyl-4-nitrobenzyl)-3-(trifluoromethyl)-1H-pyrazol-5-ol (3.01 g) and 2,4-bis(4-methoxyphenyl)-1,3-dithia-2,4-diphosphetane-2,4-disulfide (Lawson reagent) (2.50 g) was heated to reflux in toluene (20 mL) for 12 hours. After the reaction was completed, the solvent was distilled off, and the crude product was purified with silica gel column chromatography (mixed solvent of n-hexane and ethyl acetate) to obtain 1-(3-methyl-4-nitrobenzyl)-3-(trifluoromethyl)-1H-pyrazole-5-thiol (0.8 g). This crude product was dissolved in DMF (40 mL), and placed into a pressure-resistant vessel made of glass with disodium hydrogen phosphate (1.52 g), sodium hydrosulfite (0.81 g) and water (20 mL), and trifluoromethane iodide (1.48 g) was also put in the vessel. After the vessel was tightly sealed, the reaction mixture was stirred at room temperature for 7 hours. After the reaction, water was added, and the reaction mixture was extracted with ethyl acetate. The organic phase was washed with water and dried over anhydrous sodium sulfate. The solvent was distilled off, and the crude product was purified with silica gel column chromatography (mixed solvent of n-hexane and ethyl acetate) to obtain 1-(3-methyl-4-nitrobenzyl)-3-(trifluoromethyl)-5-(trifluoromethylthio)-1H-pyrazole (0.26 g).

¹H-NMR (CDCl₃, δ ppm): 2.59 (3H, s), 5.58 (2H, s), 7.03 (1H, s), 7.16-7.21 (2H, m), 7.95 (1H, d).

### SYNTHESIS EXAMPLE 50-2

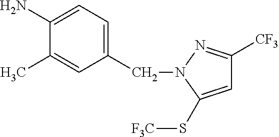

As similar to example 15-2, from (3-methyl-4-nitrobenzyl)-3-(trifluoromethyl)-5-(trifluoromethylthio)-1H-pyrazole (0.26 g), ammonium acetate (6.6 g), acetone (9 mL), water (5 mL) and 20% titanium trichloride aqueous solution (4.6 g), 2-methyl-4-({3-(trifluoromethyl)-5-[(trifluoromethyl)thio]-1H-pyrazol-1-yl}methyl)aniline (0.16 g) was obtained.

¹H-NMR (CDCl₃, δ ppm): 2.13 (3H, s), 3.64 (2H, bs), 5.39 (2H, s), 6.60 (1H, d), 6.93 (1H, s), 6.96-7.0 (2H, m).

### SYNTHESIS EXAMPLE 51-1

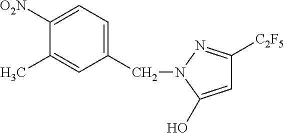

A mixture of (3-methyl-4-nitrobenzyl)-hydrazine (4.55 g) and ethyl pentafluoropropionylacetate (5.00 g) was heated to reflux in acetic acid (60 mL) for 8 hours. After cooled to room temperature, the solvent was distilled off, water and dilute hydrochloric acid were added to the crude product, and the product was extracted with ethyl acetate. The organic phase was washed with saturated brine, and dried over anhydrous sodium sulfate. The solvent was distilled off, and the resulting crude product was purified with silica gel column chromatography (mixed solvent of n-hexane and ethyl acetate) to obtain 1-(3-methyl-4-nitrobenzyl)-3-(pentafluoroethyl)-1H-pyrazol-5-ol (4.09 g).

¹H-NMR (DMSO-d₆, δ ppm): 2.50 (3H, s), 5.28 (3H, s), 5.80 (1H, s), 7.16 (1H, d), 7.27 (1H, s), 7.98 (1H, d), 12.08 (1H, s).

### SYNTHESIS EXAMPLE 51-2

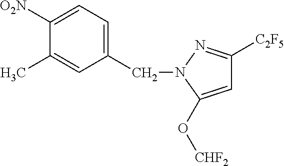

As similar to SYNTHESIS EXAMPLE 49-2, from 1-(3-methyl-4-nitrobenzyl)-3-(pentafluoroethyl)-1H-pyrazol-5-ol (1.51 g), potassium carbonate (0.89 g) and chlorodifluoromethane (1.00 g), 1-(3-methyl-4-nitrobenzyl)-5-(difluoromethoxy)-3-(pentafluoroethyl)-1H-pyrazole (1.67 g) was obtained.

¹H-NMR (CDCl₃, δ ppm): 2.58 (3H, s), 5.32 (2H, s), 6.25 (1H, s), 6.53 (1H, t), 7.15-7.18 (2H, m), 7.96 (1H, d).

### SYNTHESIS EXAMPLE 51-3

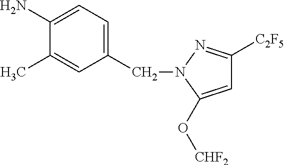

As similar to SYNTHESIS EXAMPLE 17-2, from 1-(3-methyl-4-nitrobenzyl)-5-(difluoromethoxy)-3-(pentafluoroethyl)-1H-pyrazole (1.50 g), ammonium acetate (28.82 g), acetone (55 mL), water (30 mL) and 20% titanium trichloride aqueous solution (25.9 g), 4-{[5-(difluoromethoxy)-3-(pentafluoroethyl)-1H-pyrazol-1-yl]methyl}-2-methylaniline (0.80 g) was obtained.

¹H-NMR (CDCl₃, δ ppm): 2.12 (3H, s), 3.65 (2H, bs), 5.13 (2H, s), 6.15-6.62 (3H, m), 6.94-7.19 (2H, m).

### SYNTHESIS EXAMPLE 52-1

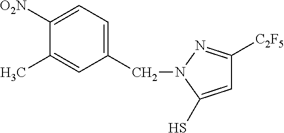

A mixture of 1-(3-methyl-4-nitrobenzyl)-3-(pentafluoroethyl)-1H-pyrazol-5-ol (2.00 g) and 2,4-bis(4-methoxyphenyl)-1,3-dithia-2,4-diphosphetane-2,4-disulfide (Lawson reagent) (1.38 g) was heated to reflux in toluene (20 mL) for 12 hours. After the reaction was completed, the solvent was distilled off, and the crude product was purified with silica gel column chromatography (mixed solvent of n-hexane and ethyl acetate) to obtain 1-(3-methyl-4-nitrobenzyl)-3-(pentafluoroethyl)-1H-pyrazole-5-thiol (0.97 g).

¹H-NMR (CDCl₃, δ ppm): 2.57 (3H, s), 5.47 (2H, s), 6.59 (1H, s), 7.09-7.15 (2H, m), 7.94 (1H, d).

### SYNTHESIS EXAMPLE 52-2

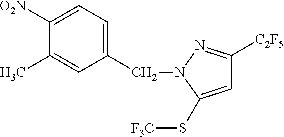

1-(3-Methyl-4-nitrobenzyl)-3-(pentafluoroethyl)-1H-pyrazole-5-thiol (0.97 g) was dissolved in DMF (20 mL), and the mixture was put into a pressure-resistant vessel made of glass with potassium carbonate (0.55 g), and after trifluoromethane iodide (1.48 g) was added, the vessel was tightly sealed, and the mixture was heated and stirred at 90° C. for 6 hours. After the reaction was completed, water was added, and the mixture was extracted with ethyl acetate. The organic phase was washed with water and dried over anhydrous magnesium sulfate. The solvent was distilled off, and the crude product was purified with silica gel column chromatography (mixed solvent of n-hexane and ethyl acetate) to obtain (3-methyl-4-nitrobenzyl)-3-(pentafluoroethyl)-5-[(trifluoromethyl)thio]-1H-pyrazole (0.05 g).

¹H-NMR (CDCl₃, δ ppm): 2.58 (3H, s), 5.60 (2H, s), 7.06 (1H, s), 7.15-7.18 (2H, m), 7.96 (1 μl, d).

### SYNTHESIS EXAMPLE 52-3

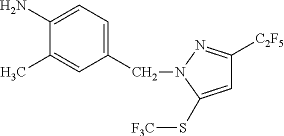

As similar to SYNTHESIS EXAMPLE 17-2, from (3-methyl-4-nitrobenzyl)-3-(pentafluoroethyl)-5-[(trifluoromethyl)thio]-1H-pyrazole (0.05 g), ammonium acetate (0.9 g), acetone (1.7 mL), water (0.9 mL) and 20% titanium trichloride aqueous solution (0.80 g), 2-methyl-4-({3-(pentafluoro-ethyl)}-5-[(trifluoromethyl)thio]-1H-pyrazol-1-yl)methyl)aniline (0.16 g) was obtained.

¹H-NMR (CDCl₃, δ ppm): 2.12 (3H, s), 3.63 (2H, bs), 5.42 (2H, s), 6.60 (1H, d), 6.95-7.23 (3H, m).

### SYNTHESIS EXAMPLE 53

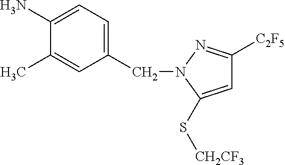

As similar to SYNTHESIS EXAMPLE 50-1, from 1-(3-methyl-4-nitrobenzyl)-3-(pentafluoroethyl)-1H-pyrazol-5-ol (3.01 g) and 2,4-bis(4-methoxyphenyl)-1,3-dithia-2,4-diphosphetane-2,4-disulfide (Lawson reagent) (2.50 g), 1-(3-methyl-4-nitrobenzyl)-3-(pentafluoroethyl)-1H-pyrazole-5-thiol (1.98 g) was obtained. From the crude product, disodium hydrogen phosphate (1.45 g), hydrosulfite sodium (0.97 g), water (9 mL) and 1,1,1-trifluoro-2-iodine ethane (0.76 g), 1-(3-methyl-4-nitrobenzyl)-3-(pentafluoroethyl)-5-[(2,2,2-trifluoroethyl)thio]-1H-pyrazole (1.15 g) was obtained, and the next reaction was conducted without purification.

To a mixture of crude 1-(3-methyl-4-nitrobenzyl)-3-(pentafluoroethyl)-5-[(2,2,2-trifluoroethyl)thio]-1H-pyrazole (1.15 g), ammonium acetate (15.8 g), acetone (30 mL) and water (16 mL), 20% titanium trichloride aqueous solution (14.2 g) was added, and the mixture was stirred at room temperature for 7 hours. After the reaction was completed, the reaction mixture was extracted with dichloromethane. The organic phase was washed sequentially with saturated aqueous solution of sodium hydrogen carbonate and saturated brine, and dried over anhydrous sodium sulfate. After the solvent was distilled off, the crude product was subjected to silica gel column chromatography (mixed solvent of n-hexane and ethyl acetate) to obtain 2-methyl-4-({3-(pentafluoroethyl)-5-[(2,2,2-trifluoroethyl)thio]-1H-pyrazol-1-yl}methyl)aniline (0.10 g).

¹H-NMR (CDCl₃, δ ppm): 2.12 (3H, s), 2.97 (3H, q), 3.64 (2H, bs), 5.40 (2H, s), 6.61 (1H, d), 6.77 (1H, s), 6.91-6.96 (2H, m).

### SYNTHESIS EXAMPLE 54-1

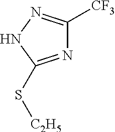

To a DMF solution (4 mL) of 3-(trifluoromethyl)-1H-1,2,4-triazole-5-thiol (0.60 g), cesium carbonate (1.27 g) was added, and then a DMF solution (2 mL) of ethyl iodide (0.55 g) was added dropwise over 5 minutes, and the mixture was stirred at room temperature for 9 hours. 10% Hydrochloric acid aqueous solution (5 mL) and water (20 mL) were added to the reaction mixture, which was then extracted with ethyl acetate. The organic phase was washed with saturated brine and dried over anhydrous magnesium sulfate. After the solvent was distilled off, the resulting crude product was purified with silica gel column chromatography (mixed solvent of cyclohexane and ethyl acetate) to obtain 5-(ethylthio)-3-(trifluoromethyl)-1H-1,2,4-triazole (0.42 g).

¹H-NMR (DMSO-d₆, δ ppm): 1.32 (311, t), 3.05-3.25 (1H, m), 3.20 (2H, q).

### SYNTHESIS EXAMPLE 54-2

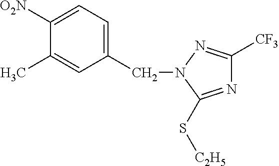

As similar to SYNTHESIS EXAMPLE 1-3, from 3-methyl-4-nitrobenzyl chloride (0.20 g), 5-(ethylthio)-3-(trifluoromethyl)-1H-1,2,4-triazole (0.22 g) and cesium carbonate (0.47 g), 5-(ethylthio)-1-(3-methyl-4-nitrobenzyl)-3-(trifluoromethyl)-1H-1,2,4-triazole (0.19 g) was obtained.

¹H-NMR (DMSO-d₆, δ ppm): 1.32 (3H, t), 3.25 (2H, q), 5.49 (2H, s), 7.26 (1H, d), 7.37 (1H, s), 7.97 (1H, d).

### SYNTHESIS EXAMPLE 54-3

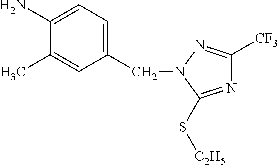

As similar to SYNTHESIS EXAMPLE 1-4, 5-(ethylthio)-1-(3-methyl-4-nitrobenzyl)-3-(trifluoromethyl)-1H-1,2,4-triazole (0.47 g), tin (II) chloride dihydrate (1.28 g), concentrated hydrochloric acid (2.8 mL) and ethanol (7 mL), 4-{[5-(ethylthio)-3-(trifluoromethyl)-1H-1,2,4-triazol-1-yl]methyl}-2-methylaniline (0.26 g) was obtained.

¹H-NMR (CD₃CN, δ ppm): 1.36 (3H, t), 3.21 (2H, q), 2.08 (3H, s), 3.95-4.10 (2H, m), 5.12 (2H, s), 6.61 (1H, d), 6.90 (1H, d), 6.95 (1H, s).

### SYNTHESIS EXAMPLE 55-1

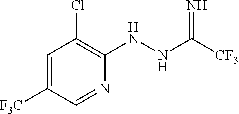

As similar to SYNTHESIS EXAMPLE 32-1, from 3-chloro-5-(trifluoromethyl)pyridin-2-yl-hydrazine (2.00 g) and trifluoromethylacetamidine (1.06 g), N′-[3-chloro-5-(trifluoromethyl)pyridin-2-yl]-2,2,2-trifluoroethanimide hydrazide (3.00 g) was obtained.

¹H-NMR (CDCl₃, δ ppm): 4.92-5.14 (1H, brs), 5.34 (1H, s), 7.40-7.75 (1H, brs) 8.30 (1H, s), 8.83 (1H, s).

### SYNTHESIS EXAMPLE 55-2

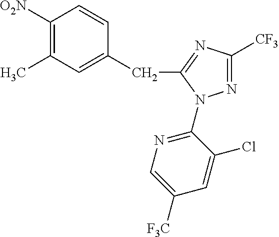

To a dioxane solution (20 mL) of 4-nitro-3-methylphenyl acetic acid (0.83 g), and N′-[3-chloro-5-(trifluoromethyl)pyridin-2-yl]-2,2,2-trifluoroethanimide hydrazide (1.50 g), N-(3-dimethylaminopropyl)-N′-ethylcarbodiimide hydrochloride (0.94 g) was added, and the mixture was heated to reflux for 48 hours. The reaction mixture was left to cool to room temperature, water was added, and the mixture was extracted with ethyl acetate and dried over anhydrous magnesium sulfate. After the solvent was distilled off, the resulting residue was dissolved in toluene (40 mL) without purification. Then, acetic acid (4 mL) was added and heated to reflux for 5 hours. After the reaction was completed, the reaction mixture was cooled to room temperature, water was added, and the mixture was extracted with ethyl acetate and dried over anhydrous magnesium sulfate. After the solvent was distilled off, the resulting crude product was purified with silica gel column chromatography (mixed solvent of n-hexane and ethyl acetate) to obtain 3-chloro-2-[5-(3-methyl-4-nitrobenzyl)-3-(trifluoromethyl)-1H-1,2,4-triazol-1-yl]-5-(trifluoromethyl)pyridine (0.65 g).

¹H-NMR (CDCl₃, δ ppm): 2.52 (3H, s), 4.33 (2H, s), 7.05 (2H, d), 7.84 (1H, d), 8.50 (1H, s), 8.77 (1H, s).

### SYNTHESIS EXAMPLE 55-3

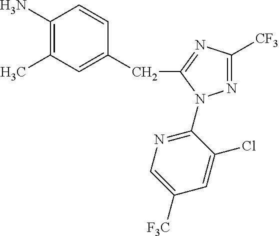

As similar to SYNTHESIS EXAMPLE 1-4, from 3-chloro-2-[5-(3-methyl-4-nitrobenzyl)-3-(trifluoromethyl)-1H-1,2,4-triazol-1-yl]-5-(trifluoromethyl)pyridine (0.50 g), tin (II) chloride dihydrate (5.37 g), concentrated hydrochloric acid (1.2 mL) and ethanol (2 mL), 4-({1-[3-chloro-5-(trifluoromethyl)pyridin-2-yl]-3-(trifluoromethyl)-1H-1,2,4-triazol-5-yl}methyl)-2-methylaniline (0.45 g) was obtained.

¹H-NMR (CDCl₃, δ ppm): 2.00 (3H, s), 3.48 (2H, br), 4.16 (2H, s), 6.38 (1H, d), 6.54 (2H, d), 7.98 (1H, s), 8.71 (1H, s).

### SYNTHESIS EXAMPLE 56-1

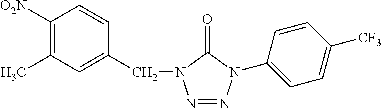

As similar to SYNTHESIS EXAMPLE 1-3, from 3-methyl-4-nitrobenzyl chloride (1.60 g), 1-(4-trifluoromethylphenyl)-1,4-dihydrotetrazol-5-one (2.00 g) and potassium carbonate (1.40 g), 1-(3-methyl-4-nitrobenzyl)-4-(4-trifluoromethylphenyl)-1,4-dihydrotetrazol-5-one (2.60 g) was obtained.

¹H-NMR (CDCl₃, δ ppm): 2.6 (3H, s), 5.3 (2H, s), 7.4-8.3 (7H, m).

### SYNTHESIS EXAMPLE 56-2

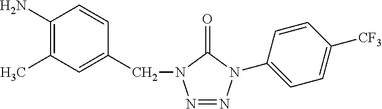

To an ethanol solution (100 mL) of 1-(3-methyl-4-nitrobenzyl)-4-(4-trifluoromethylphenyl)-1,4-dihydrotetrazol-5-one (9.48 g), 10% palladium-carbon (0.25 g) was added, and the mixture was stirred under hydrogen atmosphere at room temperature for 6 hours. After the reaction was completed, the palladium-carbon was filtered off, and the solvent was distilled off under reduced pressure to obtain 1-(4-amino-3-methylbenzyl)-4-[4-(trifluoromethyl)phenyl]-1,4-dihydro-5H-tetrazol-5-one (8.11 g).

¹H-NMR (CDCl₃, δ ppm): 2.13 (3H, s), 3.30-4.06 (2H, m), 5.03 (2H, s), 6.60 (1H, d), 7.00-7.26 (2H, m), 7.70 (2H, d), 8.10 (2H, d).

### SYNTHESIS EXAMPLE 56-3

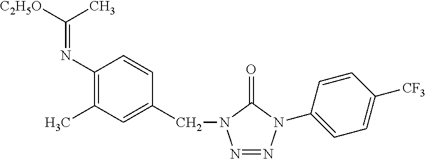

To a triethyl orthoacetate (4.87 g) solution of 1-(4-amino-3-methylbenzyl)-4-[4-(trifluoromethyl)phenyl]-1,4-dihydro-5H-tetrazol-5-one (0.52 g), p-toluenesulfonic acid monohydrate (0.014 g) was added, then the mixture was heated and stirred at 60° C. for 30 minutes. After the reaction was completed, the solvent was distilled off to obtain ethyl N-[2-methyl-4-({5-oxo-4-[4(trifluoromethyl)phenyl]-4,5-dihydro-1H-tetrazole-1-yl}methyl)phenyl]ethanimidate (0.60 g).

¹H-NMR (CDCl₃, δ ppm): 1.33 (3H, t), 1.73 (3H, s), 2.10 (3H, s), 4.23 (2H, q), 5.07 (2H, s), 6.50-7.25 (3H, m), 7.63 (2H, d), 8.06 (2H, d).

### SYNTHESIS EXAMPLE 56-4

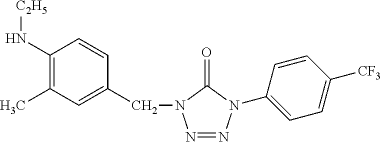

To an acetic acid (6 mL) solution of ethyl N-[2-methyl-4-({5-oxo-4-[4(trifluoromethyl)phenyl]-4,5-dihydro-1H-tetrazol-1-yl}methyl)phenyl]ethanimidate (0.60 g), sodium cyanoborohydride (0.19 g) was added in 3 portions at room temperature for 30 minutes. After stirred at room temperature for 50 minutes, the reaction mixture was diluted with ethyl acetate, then washed sequentially with water and saturated aqueous solution of sodium hydrogen carbonate, and dried over anhydrous sodium sulfate. The crude product obtained after the solvent was distilled off was washed with petroleum ether to obtain 1-[4-(ethylamino)-3-methyl benzyl]-4-[4-(trifluoromethyl)phenyl]-1,4-dihydro-5H-tetrazol-5-one (0.44 g).

¹H-NMR (CDCl₃, δ ppm): 1.26 (3H, t), 2.07 (3H, s), 3.13 (2H, q), 4.95 (2H, s), 6.47 (1H, d), 6.97-7.27 (2H, m), 7.63 (2H, d), 8.03 (2H, d).

### SYNTHESIS EXAMPLE 57-1

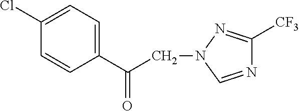

4-chlorophenacyl bromide (7.00 g) and 3-(trifluoromethyl)-1H-1,2,4-triazole (4.32 g) and potassium carbonate (4.97 g) were stirred in acetonitrile (50 mL) at 60° C. for 2 hours. After the reaction was completed, the mixture was diluted with ethyl acetate, and washed sequentially with water and saturated brine, and dried over anhydrous sodium sulfate. The solvent was distilled off, followed by the purification with the solvent, to obtain 1-(4-chlorophenyl)-2-[3-(trifluoromethyl)-1H-1,2,4-triazol-1-yl]ethanone (7.56 g).

¹H-NMR (CDCl₃, δ ppm): 5.71 (2H, s), 7.55 (2H, d), 7.93 (2H, d), 8.35 (1H, s).

### SYNTHESIS EXAMPLE 57-2

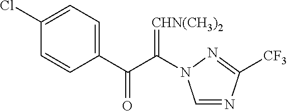

A m-xylene solution (12 mL) of 1-(4-chlorophenyl)-2-[3-(trifluoromethyl)-1H-1,2,4-triazol-1-yl]ethanone (1.30 g) and N,N-dimethylformamide dimethyl acetal (0.64 g) was heated to reflux for 2 hours. After cooled to room temperature, the solvent was distilled off to obtain the crude product of 1-(4-chlorophenyl)-3-(dimethylamino)-2-[3-(trifluoromethyl)-1H-1,2,4-triazol-1-yl]propan-2-en-1-one (1.39 g).

¹H-NMR (CDCl₃, δ ppm): 2.48 (3H, s), 3.16 (3H, s), 7.32-7.41 (4H, m), 7.48 (1H, s), 8.13 (1H, s).

### SYNTHESIS EXAMPLE 57-3

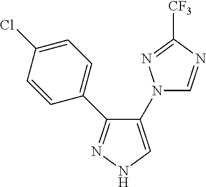

To an ethanol solution (10 mL) of 1-(4-chlorophenyl)-3-(dimethylamino)-2-[3-(trifluoromethyl)-1H-1,2,4-triazol-1-yl]propan-2-en-1-one (1.30 g), hydrazine hydrate (0.28 g) was added, then the mixture was heated to reflux for 4 hours. After cooled to room temperature, the solvent was distilled off, and the crude product was subjected to silica gel column chromatography (mixed solvent of n-hexane and ethyl acetate) to obtain 143-(4-chlorophenyl)-1H-pyrazol-4-yl]-3-(trifluoromethyl)-1H-1,2,4-triazole (1.31 g).

¹H-NMR (CDCl₃, δ ppm): 7.27-7.40 (4H, m), 7.95 (1H, s), 8.18 (1H, s), 11.18 (1H, bs).

### SYNTHESIS EXAMPLE 57-4

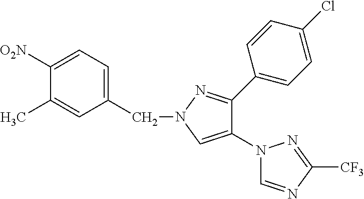

As similar to SYNTHESIS EXAMPLE 1-3, from 3-methyl-4-nitrobenzyl chloride (0.59 g), 143-(4-chlorophenyl)-1H-pyrazol-4-yl]-3-(trifluoromethyl)-1H-1,2,4-triazole (1.00 g) and potassium carbonate (0.53 g), 143-(4-chlorophenyl)-1-(3-methyl-4-nitrobenzyl)-1H-pyrazol-4-yl]-3-(trifluoromethyl)-1H-1,2,4-triazole (1.50 g) was obtained.

¹H-NMR (CDCl₃, δ ppm): 2.61 (3H, s), 5.41 (2H, s), 7.28-7.36 (4H, m), 7.84 (1H, s), 7.99 (1H, d), 8.16 (1H, s).

### SYNTHESIS EXAMPLE 57-5

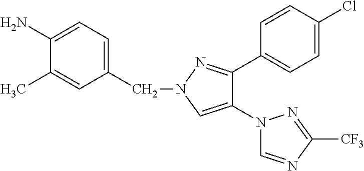

As similar to SYNTHESIS EXAMPLE 1-4, from 1-[3-(4-chlorophenyl)-1-(3-methyl-4-nitrobenzyl)-1H-pyrazol-4-yl]-3-(trifluoromethyl)-1H-1,2,4-triazole (0.60 g), tin (II) chloride dihydrate (1.46 g), concentrated hydrochloric acid (1.5 mL) and ethanol (3 mL), 4-({3-(4-chlorophenyl)-4-[3-(trifluoromethyl)-1H-1,2,4-triazol-1-yl]-1H-pyrazol-1-yl]methyl)-2-methylaniline (0.63 g) was obtained.

¹H-NMR (CDCl₃, δ ppm): 2.17 (3H, s), 3.73 (2H, bs), 5.22 (2H, s), 6.68 (1H, d), 7.04-7.07 (2H, m), 7.28-7.35 (4H, m), 7.60 (1H, s), 8.10 (1H, s).

### SYNTHESIS EXAMPLE 58-1

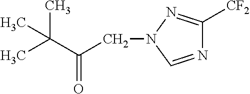

As similar to SYNTHESIS EXAMPLE 18-2, from 1-bromopinacolone (0.90 g), 3-(trifluoromethyl)-1H-1,2,4-triazole (0.62 g) and triethylamine (0.63 mL), 3,3-dimethyl-1-[3-(trifluoromethyl)-1H-1,2,4-triazol-1-yl]butan-2-one (1.22 g) was obtained.

¹H-NMR (CDCl₃, δ ppm): 1.30 (9H, s), 5.25 (2H, s), 8.24 (1H, s).

### SYNTHESIS EXAMPLE 58-2

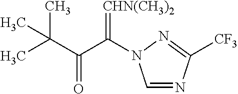

As similar to SYNTHESIS EXAMPLE 57-2, from 3,3-dimethyl-1-[3-(trifluoromethyl)-1H-1,2,4-triazol-1-yl]butan-2-one (1.11 g) and N,N-dimethylformamide dimethylacetal (0.67 g), 1-(dimethylamino)-4,4-di-methyl-2-[3-(trifluoromethyl)-1H-1,2,4-triazol-1-yl]pentan-1-en-3-one (1.25 g) was obtained as an E/Z-isomer mixture.

¹H-NMR (CDCl₃, δ ppm): 1.07 (9H, s), 2.50-2.96 (6H, m), 7.32-7.41 (4H, m), 7.74 and 8.16 (1H, s).

### SYNTHESIS EXAMPLE 58-3

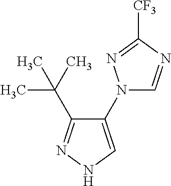

As similar to SYNTHESIS EXAMPLE 57-3, from 1-(dimethylamino)-4,4-dimethyl-2-[3-(trifluoromethyl)-1H-1,2,4-triazol-1-yl]pentan-1-en-3-one (1.10 g) and hydrazine hydrate (0.29 g), 1-(3-t-butyl-1H-pyrazol-4-yl)-3-(trifluoromethyl)-1H-1,2,4-triazole (0.72 g) was obtained.

¹H-NMR (CDCl₃, δ ppm): 1.25 (9H, s), 4.75-5.55 (1H, m), 7.68 (1H, s), 8.27 (1H, s).

### SYNTHESIS EXAMPLE 58-4

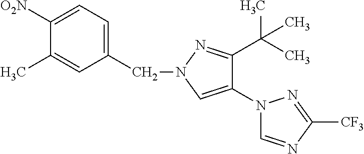

As similar to SYNTHESIS EXAMPLE 1-3, from 3-methyl-4-nitrobenzyl chloride (0.37 g), 1-(3-t-butyl-1H-pyrazol-4-yl)-3-(trifluoromethyl)-1H-1,2,4-triazole (0.51 g) and potassium carbonate (0.33 g), 1-[3-t-butyl-1-(3-methyl-4-nitrobenzyl)-1H-pyrazol-4-yl]-3-(trifluoromethyl)-1H-1,2,4-triazole (0.91 g) was obtained.

¹H-NMR (CDCl₃, δ ppm): 1.20 (9H, s), 2.61 (3H, s), 5.30 (2H, s), 7.20-7.29 (2H, m), 7.57 (1H, s), 7.99 (1H, d), 8.23 (1H, s).

### SYNTHESIS EXAMPLE 58-5

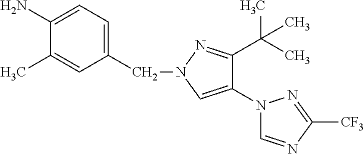

As similar to SYNTHESIS EXAMPLE 1-4, from 1-[3-t-butyl-1-(3-methyl-4-nitrobenzyl)-1H-pyrazol-4-yl]-3-(trifluoromethyl)-1H-1,2,4-triazole (0.85 g), tin (II) chloride dihydrate (2.35 g), concentrated hydrochloric acid (3.5 mL), and ethanol (4 mL), 4-({3-t-butyl-4-[3-(trifluoromethyl)-1H-1,2,4-triazol-1-yl]-1H-pyrazol-1-yl}methyl)-2-methylaniline (0.75 g) was obtained.

¹H-NMR (CDCl₃, δ ppm): 1.19 (9H, s), 2.16 (3H, s), 3.64-3.78 (2H, m), 5.11 (2H, s), 6.66 (1H, d), 7.00 (1H, d), 7.03 (1H, s), 7.32 (1H, s), 8.17 (1H, s).

### SYNTHESIS EXAMPLE 59-1

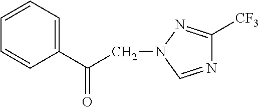

As similar to SYNTHESIS EXAMPLE 57-1, from phenacyl bromide (1.55 g), 3-(trifluoromethyl)-1H-1,2,4-triazole (1.51 g) and potassium carbonate (1.66 g), 1-phenyl-2-[3-(trifluoromethyl)-1H-1,2,4-triazol-1-yl]ethanone (2.20 g) was obtained.

¹H-NMR (CDCl₃, δ ppm): 5.74 (2H, s), 7.53-7.74 (3H, m), 7.99 (2H, d), 8.35 (1H, s).

### SYNTHESIS EXAMPLE 59-2

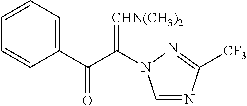

As similar to SYNTHESIS EXAMPLE 57-2, from 1-phenyl-2-[3-(trifluoromethyl)-1H-1,2,4-triazol-1-yl]ethanone (1.60 g) and N,N-dimethylformamide dimethylacetal (0.89 g), 1-phenyl-3-(dimethylamino)-2-[3-(trifluoromethyl)-1H-1,2,4-triazol-1-yl]propan-2-en-1-one (2.02 g) was obtained.

¹H-NMR (CDCl₃, δ ppm): 2.28-3.36 (6H, m), 7.14-7.61 (6H, m), 8.16 (1H, s).

### SYNTHESIS EXAMPLE 59-3

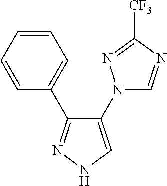

As similar to SYNTHESIS EXAMPLE 57-3, from 1-phenyl-3-(dimethylamino)-2-[3-(trifluoromethyl)-1H-1,2,4-triazol-1-yl]propan-2-en-1-one (1.10 g) and hydrazine hydrate (0.24 g), 1-[3-phenyl-1H-pyrazol-4-yl]-3-(trifluoromethyl)-1H-1,2,4-triazole (1.20 g) was obtained.

¹H-NMR (CDCl₃, δ ppm): 4.77-5.10 (1H, m), 7.29-7.50 (5H, m), 7.91 (1H, s), 8.15 (1H, s).

### SYNTHESIS EXAMPLE 59-4

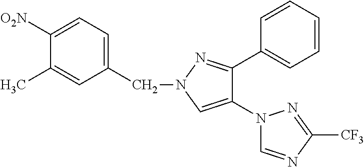

As similar to SYNTHESIS EXAMPLE 1-3, from 3-methyl-4-nitrobenzylchloride (0.80 g), 1-[3-phenyl-1H-pyrazol-4-yl]-3-(trifluoromethyl)-1H-1,2,4-triazole (1.20 g) and potassium carbonate (0.71 g), 1-[3-phenyl-1-(3-methyl-4-nitrobenzyl)-1H-pyrazol-4-yl]-3-(trifluoromethyl)-1H-1,2,4-triazole (1.45 g) was obtained.

¹H-NMR (CDCl₃, δ ppm): 2.61 (3H, s), 5.41 (2H, s), 7.16-7.56 (7H, m), 7.83 (1H, s), 8.00 (1H, d), 8.12 (1H, s).

### SYNTHESIS EXAMPLE 59-5

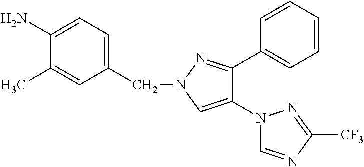

As similar to SYNTHESIS EXAMPLE 1-4, from 1-[3-phenyl-1-(3-methyl-4-nitrobenzyl)-1H-pyrazol-4-yl]-3-(trifluoromethyl)-1H-1,2,4-triazole (1.21 g), tin (II) chloride dihydrate (3.20 g), concentrated hydrochloric acid (3 mL) and ethanol (5 mL), 2-methyl-4-({3-phenyl-4-[3-(trifluoromethyl)-1H-1,2,4-triazol-1-yl]-1H-pyrazol-1-yl}methyl)aniline (1.17 g) was obtained.

¹H-NMR (CDCl₃, δ ppm): 2.17 (3H, s), 3.72 (2H, bs), 5.22 (2H, s), 6.67 (1H, d), 7.06 (1H, d), 7.07 (1H, s), 7.35 (5H, s), 7.61 (1H, s), 8.07 (1H, s).

### SYNTHESIS EXAMPLE 60-1

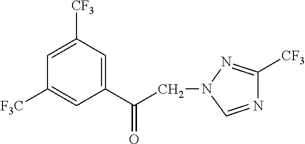

As similar to SYNTHESIS EXAMPLE 18-2, from 3,5-bis(trifluoromethyl)phenacyl bromide (1.82 g), 3-(trifluoromethyl)-1H-1,2,4-triazole (0.82 g) and triethylamine (0.84 mL), 1-[3,5-bis(trifluoromethyl)phenyl]-2-[3-(trifluoromethyl)-1H-1,2,4-triazol-1-yl]ethanone (1.48 g) was obtained.

¹H-NMR (CDCl₃, δ ppm): 5.80 (2H, s), 8.21 (1H, s), 8.35 (1H, s), 8.42 (2H, s).

### SYNTHESIS EXAMPLE 60-2

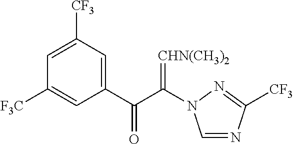

As similar to SYNTHESIS EXAMPLE 57-2, from 1-[3,5-bis(trifluoromethyl)phenyl]-2-[3-(trifluoromethyl)-1H-1,2,4-triazol-1-yl]ethanone (1.40 g) and N,N-dimethylformamide dimethylacetal (0.51 g), 1-[3,5-bis(trifluoromethyl)phenyl]-3-(dimethylamino)-2-[3-(trifluoromethyl)-1H-1,2,4-triazol-1-yl]propan-2-en-1-one (1.56 g) was obtained.

¹H-NMR (CDCl₃, δ ppm): 2.51 (3H, bs), 3.27 (3H, bs), 7.62-7.91 (5H, m), 8.04 (1H, s).

### SYNTHESIS EXAMPLE 60-3

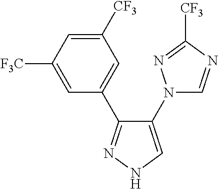

As similar to SYNTHESIS EXAMPLE 57-3, from 1-[3,5-bis(trifluoromethyl)phenyl]-3-(dimethylamino)-2-[3-(trifluoromethyl)-1H-1,2,4-triazol-1-yl]propan-2-en-1-one (1.56 g) and hydrazine hydrate (0.26 g), 1-{3-[3,5-bis(trifluoromethyl)phenyl]-1H-pyrazol-4-yl}-3-(trifluoromethyl)-1H-1,2,4-triazole (1.56 g) was obtained.

¹H-NMR (CDCl₃, δ ppm): 4.37-4.96 (1H, m), 7.86 (1H, s), 7.93 (2H, s), 8.02 (1H, s), 8.27 (1H, s).

### SYNTHESIS EXAMPLE 60-4

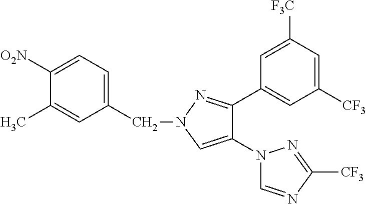

As similar to SYNTHESIS EXAMPLE 1-3, from 3-methyl-4-nitrobenzyl chloride (0.67 g), 1-{3-[3,5-bis(trifluoromethyl)phenyl]-1H-pyrazol-4-yl}-3-(trifluoromethyl)-1H-1,2,4-triazole (1.50 g) and potassium carbonate (0.60 g), 1-{3-[3,5-bis(trifluoromethyl)phenyl]-1-(3-methyl-4-nitrobenzyl)-1H-pyrazole-4-yl}-3-(trifluoromethyl)-1H-1,2,4-triazole (1.51 g) was obtained.

¹H-NMR (CDCl₃, δ ppm): 2.63 (3H, s), 5.46 (2H, s), 7.28-7.35 (2H, m), 7.83-7.94 (4H, m), 8.02 (1H, d), 8.23 (1H, s).

### SYNTHESIS EXAMPLE 60-5

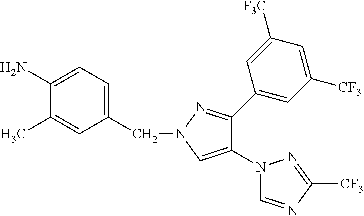

As similar to SYNTHESIS EXAMPLE 1-4, from 1-{(3-[3,5-bis(trifluoromethyl)phenyl]-1-(3-methyl-4-nitrobenzyl)-1H-pyrazol-4-yl}-3-(trifluoromethyl)-1H-1,2,4-triazole (1.30 g), tin (II) chloride dihydrate (2.60 g), concentrated hydrochloric acid (0.8 mL) and ethanol (4 mL), 4-({3-[3,5-bis(trifluoromethyl)phenyl]-4-[3-(trifluoromethyl)-1H-1,2,4-triazol-1-yl]-1H-pyrazol-1-yl}methyl)-2-methylaniline (1.20 g) was obtained.

¹H-NMR (CDCl₃, δ ppm): 2.19 (3H, s), 3.61-3.83 (2H, m), 5.27 (2H, s), 6.70 (1H, d), 6.98-7.13 (2H, m), 7.83 (1H, s), 7.90 (2H, s), 8.187 (1H, s).

### SYNTHESIS EXAMPLE 61-1

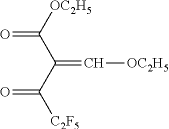

A mixture of ethyl pentafluoropropionylacetate (10.00 g), triethyl orthoformate (9.50 g) and acetic anhydride (13.08 g) was heated and stirred at 130° C. for 12 hours. After cooled to room temperature, the solvent was distilled off to obtain ethyl 2-(ethoxymethylidene)-4,4,5,5,5-pentafluoro-3-oxopentanoate (10.30 g) as an E/Z isomer mixture.

¹H-NMR (CDCl₃, δ ppm): 1.21-1.47 (6H, m), 4.17-4.41 (4H, m), 7.71 and 7.74 (1H, s).

### SYNTHESIS EXAMPLE 61-2

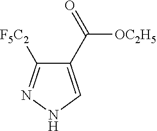

To a t-butylmethylether solution (40 mL) of 2-(ethoxymethylidene)-4,4,5,5,5-pentafluoro-3-oxopentanoate (10.30 g), hydrazine hydrate (1.79 g) was added, and the mixture was stirred at 0° C. for 4 hours. The solvent was distilled off, and the resulting crude product was washed with petroleum ether to obtain ethyl 3-(pentafluoroethyl)-1H-pyrazole-4-carboxylate (8.60 g).

¹H-NMR (CDCl₃, δ ppm): 1.37 (3H, t), 4.10-4.33 (1H, m), 4.36 (2H, q), 8.25 (1H, s).

### SYNTHESIS EXAMPLE 61-3

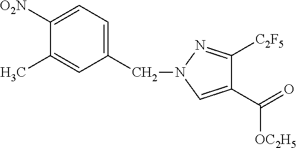

As similar to SYNTHESIS EXAMPLE 1-3, from 3-methyl-4-nitrobenzyl chloride (4.83 g), ethyl 3-(pentafluoroethyl)-1H-pyrazol-4-carboxylate (5.96 g) and potassium carbonate (4.56 g), ethyl 1-(3-methyl-4-nitrobenzyl)-3-(pentafluoroethyl)-1H-pyrazole-4-carboxylate (7.46 g) was obtained.

¹H-NMR (CDCl₃, δ ppm): 1.33 (3H, t), 2.60 (3H, s), 4.30 (2H, q), 5.39 (2H, s), 7.18-7.25 (2H, m), 7.95-8.03 (3H, m).

### SYNTHESIS EXAMPLE 61-4

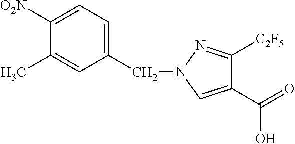

To an ethanol solution (80 mL) of ethyl 1-(3-methyl-4-nitrobenzyl)-3-(pentafluoroethyl)-1H-pyrazole-4-carboxylate (6.11 g), sodium hydroxide (0.66 g) was added, and this was heated and stirred at 50° C. for 16 hours. The solvent was distilled off, and the resulting residue was dissolved in water, washed sequentially with t-butylmethylether and hexane, and the liquor was adjusted to pH 2 or below with concentrated hydrochloric acid and extracted with ethyl acetate. The organic phase was washed with saturated brine, and dried over anhydrous sodium sulfate. The solvent was distilled off to obtain 1-(3-methyl-4-nitrobenzyl)-3-(pentafluoroethyl)-1H-pyrazole-4-carboxylic acid (5.12 g).

¹H-NMR (CDCl₃, δ ppm): 2.60 (3H, s), 5.41 (2H, s), 7.21-7.26 (2H, m), 7.37-7.92 (1H, m), 7.99 (1H, d), 8.08 (1H, s).

### SYNTHESIS EXAMPLE 61-5

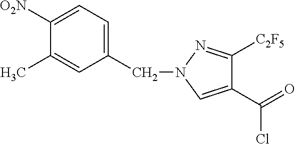

To a 1,2-dichloroethane solution (60 mL) of 1-(3-methyl-4-nitrobenzyl)-3-(pentafluoroethyl)-1H-pyrazole-4-carboxylic acid (3.00 g) and DMF (0.01 g), oxalyl chloride (1.14 g) was added at room temperature, then the mixture was heated and stirred at 60° C. until gas generation ceased. After the reaction was completed, the solvent was distilled off to obtain 1-(3-methyl-4-nitrobenzyl)-3-(pentafluoroethyl)-1H-pyrazole-4-carboxylic acid chloride (3.43 g), which was subjected to the next reaction without purification.

### SYNTHESIS EXAMPLE 61-6

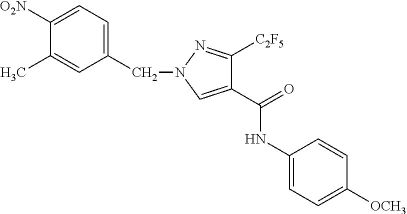

p-Anisidine (0.25 g) and triethylamine (0.28 g) are dissolved in THF (10 mL), a THF solution of 1-(3-methyl-4-nitrobenzyl)-3-(pentafluoroethyl)-1H-pyrazole-4-carboxylic acid chloride (0.79 g) was added dropwise under ice cooling, and the mixture was stirred at the same temperature for 1 hour. After 2N hydrochloric acid was added to the reaction mixture, this was extracted with ethyl acetate. The organic phase was washed sequentially with saturated aqueous solution of sodium hydrogen carbonate and saturated brine, and dried over anhydrous sodium sulfate. The solvent was distilled off, and the crude product was purified with silica gel column chromatography (mixed solvent of n-hexane and ethyl acetate) to obtain N-(4-methoxyphenyl)-1-(3-methyl-4-nitrobenzyl)-3-(pentafluoroethyl)-1H-pyrazole-4-carboxamide (0.56 g).

¹H-NMR (CDCl₃, δ ppm): 2.59 (3H, s), 3.80 (3H, s), 5.37 (2H, s), 6.88 (2H, d), 7.19-7.26 (2H, m), 7.44 (2H, d), 7.66-7.75 (1H, m), 7.97 (1H, d), 8.05 (1H, s).

### SYNTHESIS EXAMPLE 61-7

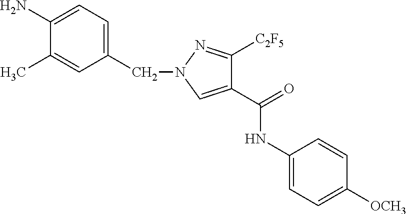

As similar to SYNTHESIS EXAMPLE 1-4, from N-(4-methoxyphenyl)-1-(3-methyl-4-nitrobenzyl)-3-(pentafluoroethyl)-1H-pyrazole-4-carboxamide (0.56 g), tin (II) chloride dihydrate (1.30 g), concentrated hydrochloric acid (1 mL) and ethanol (2 mL), 1-(4-amino-3-methylbenzyl)-N-(4-methoxyphenyl)-3-(pentafluoroethyl)-1H-pyrazole-4-carboxamide (0.51 g) was obtained.

¹H-NMR (CDCl₃, δ ppm): 2.16 (3H, s), 3.73 (2H, bs), 3.80 (3H, s), 5.19 (2H, s), 6.66 (1H, d), 6.87 (2H, d), 7.00 (1H, d), 7.02 (1H, s), 7.43 (2H, d), 7.58-7.66 (1H, m), 7.84 (1H, s).

### SYNTHESIS EXAMPLE 62-1

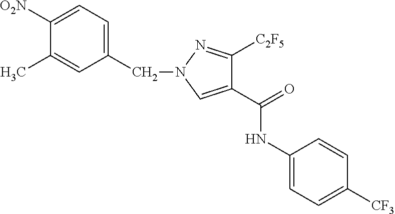

As similar to SYNTHESIS EXAMPLE 61-6, from p-aminobenzotrifluoride (0.23 g), triethylamine (0.20 g) and 1-(3-methyl-4-nitrobenzyl)-3-(pentafluoroethyl)-1H-pyrazol-4-carboxylic acid chloride (0.57 g), N-[4-(trifluoromethyl)phenyl]-1-(3-methyl-4-nitrobenzyl)-3-(pentafluoroethyl)-1H-pyrazole-4-carboxamide (0.33 g) was obtained.

¹H-NMR (CDCl₃, δ ppm): 2.61 (3H, s), 5.41 (2H, s), 7.25 (1H, d), 7.26 (1H, s), 7.61 (2H, d), 7.68 (2H, d), 7.89-7.95 (1H, m), 7.99 (1H, d), 8.09 (1H, s).

### SYNTHESIS EXAMPLE 62-2

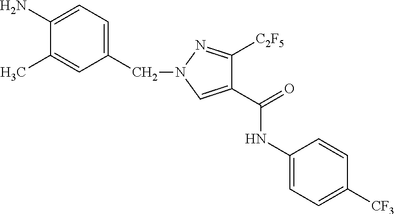

As similar to SYNTHESIS EXAMPLE 1-4, from N-[4-(trifluoromethyl)phenyl]-1-(3-methyl-4-nitrobenzyl)-3-(pentafluoroethyl)-1H-pyrazole-4-carboxamide (0.33 g), tin (II) chloride dihydrate (0.71 g), concentrated hydrochloric acid (1 mL) and ethanol (2 mL), 1-(4-amino-3-methylbenzyl)-3-(pentafluoroethyl)-N-[4-(trifluoromethyl)phenyl]-1H-pyrazole-4-carboxamide (0.28 g) was obtained.

¹H-NMR (CDCl₃, δ ppm): 2.17 (3H, s), 3.74 (2H, bs), 5.21 (2H, s), 6.68 (1H, d), 7.02 (1H, d), 7.03 (1H, s), 7.59 (2H, d), 7.67 (2H, d), 7.83-7.90 (1H, m), 7.88 (1H, s).

### SYNTHESIS EXAMPLE 63-1

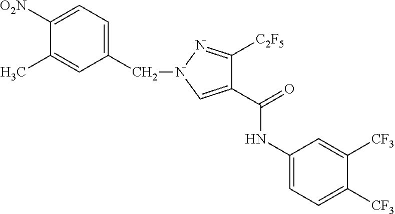

As similar to SYNTHESIS EXAMPLE 61-6, from 3,4-bis(trifluoromethyl)aniline (0.46 g), triethylamine (0.28 g) and 1-(3-methyl-4-nitrobenzyl)-3-(pentafluoroethyl)-1H-pyrazole-4-carboxylic acid chloride (0.79 g), N-[3,4-bis(trifluoromethyl)phenyl]-1-(3-methyl-4-nitrobenzyl)-3-(pentafluoroethyl)-1H-pyrazole-4-carboxamide (0.36 g) was obtained.

¹H-NMR (CDCl₃, δ ppm): 2.62 (3H, s), 5.41 (2H, s), 7.23-7.29 (2H, m), 7.84 (1H, d), 7.93 (1H, d), 7.97-8.03 (3H, m), 8.10 (1H, s).

### SYNTHESIS EXAMPLE 63-2

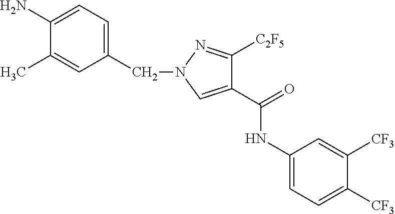

As similar to SYNTHESIS EXAMPLE 1-4, from N-[3,4-bis(trifluoromethyl)phenyl]-1-(3-methyl-4-nitrobenzyl)-3-(pentafluoroethyl)-1H-pyrazole-4-carboxamide (0.36 g), tin (II) chloride dihydrate (0.68 g), concentrated hydrochloric acid (1 mL) and ethanol (2 mL), 1-(4-amino-3-methylbenzyl)-3-(pentafluoroethyl)-N-[3,4-bis(trifluoromethyl)phenyl]-1H-pyrazole-4-carboxamide (0.35 g) was obtained.

¹H-NMR (CDCl₃, δ ppm): 2.17 (3H, s), 3.75 (2H, bs), 5.22 (2H, s), 6.68 (1H, d), 7.02 (1H, d), 7.04 (1H, s), 7.81 (1H, d), 7.87-8.01 (4H, m).

### SYNTHESIS EXAMPLE 64-1

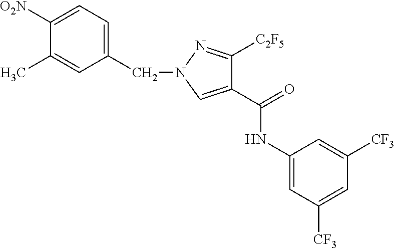

As similar to SYNTHESIS EXAMPLE 61-6, from 3,5-bis(trifluoromethyl) aniline (0.33 g), triethylamine (0.20 g) and 1-(3-methyl-4-nitrobenzyl)-3-(pentafluoroethyl)-1H-pyrazole-4-carboxylic acid chloride (0.57 g), N-[3,5-bis(trifluoromethyl)phenyl]-1-(3-methyl-4-nitrobenzyl)-3-(pentafluoroethyl)-1H-pyrazole-4-carboxamide (0.27 g) was obtained.

¹H-NMR (CDCl₃, δ ppm): 2.62 (3H, s), 5.42 (2H, s), 7.23-7.29 (2H, m), 7.67 (1H, s), 7.97-8.08 (4H, m), 8.10 (1H, s).

### SYNTHESIS EXAMPLE 64-2

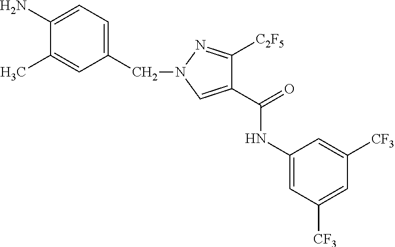

As similar to SYNTHESIS EXAMPLE 1-4, from N-[3,5-bis(trifluoromethyl)phenyl]-1-(3-methyl-4-nitrobenzyl)-3-(pentafluoroethyl)-1H-pyrazole-4-carboxamide (0.27 g), tin (II) chloride dihydrate (0.52 g), concentrated hydrochloric acid (1 mL) and ethanol (2 mL), 1-(4-amino-3-methylbenzyl)-3-(pentafluoroethyl)-N-[3,5-bis(trifluoromethyl)phenyl]-1H-pyrazole-4-carboxamide (0.23 g) was obtained.

¹H-NMR (CDCl₃, δ ppm): 2.16 (3H, s), 3.75 (2H, bs), 5.22 (2H, s), 6.68 (1H, d), 7.02 (1H, d), 7.03 (1H, s), 7.63 (1H, s), 7.88 (1H, s), 7.96-8.05 (1H, m), 8.03 (2H, s).

### SYNTHESIS EXAMPLE 65-1

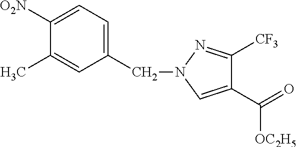

As similar to SYNTHESIS EXAMPLE 1-3, from 3-methyl-4-nitrobenzyl chloride (3.38 g), ethyl 3-(trifluoromethyl)-1H-pyrazole-4-carboxylate (4.16 g) and potassium carbonate (3.77 g), ethyl 1-(3-methyl-4-nitrobenzyl)-3-(trifluoromethyl)-1H-pyrazole-4-carboxylate (3.59 g) was obtained.

¹H-NMR (CDCl₃, δ ppm): 1.35 (3H, t), 2.61 (3H, s), 4.32 (2H, q), 5.37 (2H, s), 7.18-7.26 (2H, m), 7.95-8.03 (3H, m).

### SYNTHESIS EXAMPLE 65-2

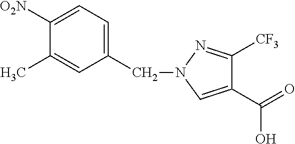

As similar to SYNTHESIS EXAMPLE 61-4, from ethyl 1-(3-methyl-4-nitrobenzyl)-3-(trifluoromethyl)-1H-pyrazole-4-carboxylate (3.50 g) and sodium hydroxide (0.43 g), 1-(3-methyl-4-nitrobenzyl)-3-(trifluoromethyl)-1H-pyrazole-4-carboxylic acid (2.94 g) was obtained.

¹H-NMR (CDCl₃, δ ppm): 2.61 (3H, s), 5.40 (2H, s), 7.20-7.29 (2H, m), 7.77-8.13 (3H, m).

### SYNTHESIS EXAMPLE 65-3

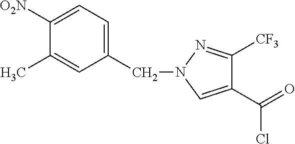

As similar to SYNTHESIS EXAMPLE 61-5, from 1-(3-methyl-4-nitrobenzyl)-3-(trifluoromethyl)-1H-pyrazole-4-carboxylic acid (2.63 g), DMF (0.01 g) and oxalyl chloride (1.52 g), 1-(3-methyl-4-nitrobenzyl)-3-(trifluoromethyl)-1H-pyrazole-4-carboxylic acid chloride (2.60 g) was obtained, and the next reaction was carried out without purification.

### SYNTHESIS EXAMPLE 65-4

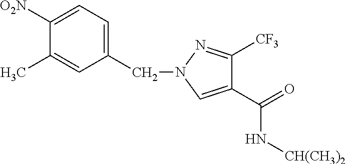

As similar to SYNTHESIS EXAMPLE 61-6, from isopropylamine (0.27 g) and 1-(3-methyl-4-nitrobenzyl)-3-(trifluoromethyl)-1H-pyrazole-4-carboxylic acid chloride (0.52 g), N-(propan-2-yl)-1-(3-methyl-4-nitrobenzyl)-3-(trifluoromethyl)-1H-pyrazole-4-carboxamide (0.22 g) was obtained.

¹H-NMR (CDCl₃, δ ppm): 1.22 (6H, d), 2.60 (3H, s), 4.14-4.28 (1H, m), 5.34 (2H, s), 5.79-5.91 (1H, m), 7.18-7.25 (2H, m), 7.93-8.00 (2H, m).

### SYNTHESIS EXAMPLE 65-5

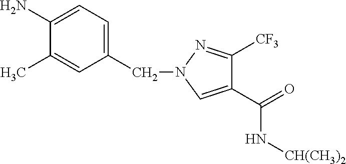

As similar to SYNTHESIS EXAMPLE 1-4, from N-(propan-2-yl)-1-(3-methyl-4-nitrobenzyl)-3-(trifluoromethyl)-1H-pyrazole-4-carboxamide (0.19 g), tin (II) chloride dihydrate (0.58 g), concentrated hydrochloric acid (0.6 mL) and ethanol (1 mL), 1-(4-amino-3-methylbenzyl)-N-(propane-2-yl)-3-(trifluoromethyl)-1H-pyrazole-4-carboxamide (0.14 g) was obtained.

¹H-NMR (CDCl₃, δ ppm): 1.20 (6H, d), 2.15 (3H, s), 4.11-4.25 (1H, m), 5.15 (2H, s), 5.73-5.85 (1H, m), 6.66 (1H, d), 6.98 (1H, d), 7.00 (1H, d), 7.76 (1H, s).

### SYNTHESIS EXAMPLE 66-1

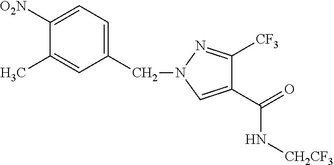

As similar to SYNTHESIS EXAMPLE 61-6, from 2,2,2-trifluoroethylamine (0.18 g), triethylamine (0.17 g) and 1-(3-methyl-4-nitrobenzyl)-3-(trifluoromethyl)-1H-pyrazole-4-carboxylic acid chloride (0.52 g), 1-(3-methyl-4-nitrobenzyl)-N-(2,2,2-trifluoroethyl)-3-(trifluoromethyl)-1H-pyrazole-4-carboxamide (0.28 g) was obtained.

¹H-NMR (CDCl₃, δ ppm): 2.60 (3H, s), 4.00-4.16 (2H, m), 5.36 (2H, s), 6.22-6.35 (1H, m), 7.20-7.28 (2H, m), 7.98 (1H, d), 8.03 (1H, s).

### SYNTHESIS EXAMPLE 66-2

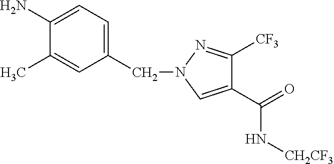

As similar to SYNTHESIS EXAMPLE 1-4, from 1-(3-methyl-4-nitrobenzyl)-N-(2,2,2-trifluoroethyl)-3-(trifluoromethyl)-1H-pyrazole-4-carboxamide (0.17 g), tin (II) chloride dihydrate (0.46 g), concentrated hydrochloric acid (0.5 mL), and ethanol (0.8 mL), 1-(4-amino-3-methylbenzyl)-N-(propan-2-yl)-3-(trifluoromethyl)-1H-pyrazole-4-carboxamide (0.14 g) was obtained.

(CDCl₃, δ ppm): 1.83-2.53 (2H, m), 2.16 (2H, s), 3.96-4.11 (2H, m), 5.17 (2H, s), 6.15-6.27 (1H, m), 6.67 (1H, d), 6.99 (1H, d), 7.00 (1H, d), 7.82 (1H, s).

### SYNTHESIS EXAMPLE 67-1

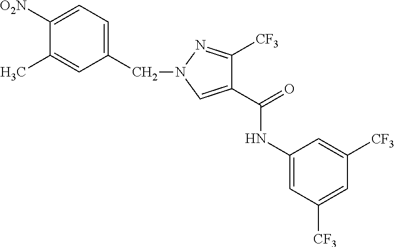

As similar to SYNTHESIS EXAMPLE 61-6, from 3,5-bis(trifluoromethyl)aniline (0.37 g), triethylamine (0.17 g) and 1-(3-methyl-4-nitrobenzyl)-3-(trifluoromethyl)-1H-pyrazole-4-carboxylic acid chloride (0.52 g), N-[3,5-bis(trifluoromethyl)phenyl]-1-(3-methyl-4-nitrobenzyl)-3-(trifluoromethyl)-1H-pyrazole-4-carboxamide (0.12 g) was obtained.

¹H-NMR (CDCl₃, δ ppm): 2.56 (3H, s), 5.42 (2H, s), 7.203-7.29 (2H, m), 7.61 (1H, s), 7.94 (1H, d), 8.00 (2H, s), 8.15 (1H, s), 8.21 (1H, bs).

### SYNTHESIS EXAMPLE 67-2

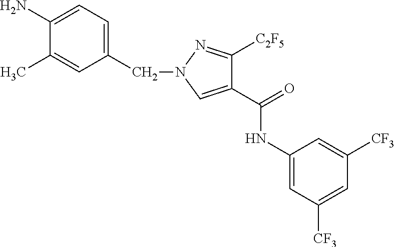

As similar to SYNTHESIS EXAMPLE 1-4, from N-[3,5-bis(trifluoromethyl)phenyl]-1-(3-methyl-4-nitrobenzyl)-3-(trifluoromethyl)-1H-pyrazole-4-carboxamide (0.10 g), tin (II) chloride dihydrate (0.21 g), concentrated hydrochloric acid (0.2 mL) and ethanol (0.4 mL), 1-(4-amino-3-methylbenzyl)-3-(trifluoromethyl)-N-[3,5-bis(trifluoromethyl)phenyl]-1H-pyrazole-4-carboxamide (0.08 g) was obtained.

¹H-NMR (CDCl₃, δ ppm): 2.16 (3H, s), 3.76 (2H, bs), 5.20 (2H, s), 5.20 (2H, s), 6.67 (1H, d), 7.00 (1H, d), 7.03 (1H, s), 7.63 (1H, s), 7.88 (1H, s), 7.97 (1H, bs), 8.03 (2H, s).

### SYNTHESIS EXAMPLE 68-1

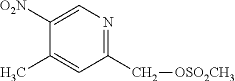

To a dichloromethane (10 mL) solution of (4-methyl-5-nitropyridin-2-yl)methanol (0.37 g), triethylamine (0.44 mL) was added, and the mixture was cooled to 0° C. Then, a dichloromethane (10 mL) solution of methane sulfonyl chloride (0.28 g) was added dropwise over 30 minutes. After the reaction was completed, the reaction mixture was washed sequentially with saturated aqueous solution of sodium hydrogen carbonate, 10% hydrochloric acid and saturated brine, and dried over anhydrous magnesium sulfate. The solvent was distilled off to obtain (4-methyl-5-nitropyridine-2-yl)methyl methanesulfonate (0.50 g).

logP(acid): 1.49

### SYNTHESIS EXAMPLE 68-2

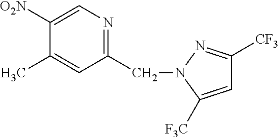

As similar to SYNTHESIS EXAMPLE 17-1, from (4-methyl-5-nitropyridin-2-yl)methyl methanesulfonate (0.50 g), 3,5-bis(trifluoromethyl)-1H-pyrazole (0.41 g) and potassium carbonate (0.34 g), 2-{[3,5-bis(trifluoromethyl)-1H-pyrazol-1-yl]methyl}-4-methyl-5-nitropyridine (0.61 g) was obtained.

logP(acid): 3.64

### SYNTHESIS EXAMPLE 68-3

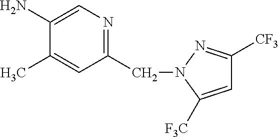

As similar to SYNTHESIS EXAMPLE 1-4, from 2-{[3,5-bis(trifluoromethyl)-1H-pyrazol-1-yl]methyl}-4-methyl-5-nitropyridine (0.60 g), tin (II) chloride dihydrate (1.60 g), concentrated hydrochloric acid (4 mL) and ethanol (10 mL), 6-{[3,5-bis(trifluoromethyl)-1H-pyrazol-1-yl]methyl}-4-methylpyridine-3-amine (0.54 g) was obtained.

¹H-NMR (CD₃CN, δ ppm): 2.11 (3H, s), 4.11 (2H, bs), 5.42 (2H, s), 6.86 (1H, s), 7.10 (1H, s), 7.86 (1H, s).

### SYNTHESIS EXAMPLE 69-1

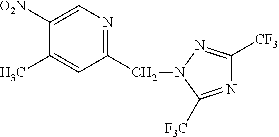

As similar to SYNTHESIS EXAMPLE 17-1, from (4-methyl-5-nitropyridine-2-yl)methyl methanesulfonate (0.17 g), 3,5-bis(trifluoromethyl)-1H-1,2,4-triazole (0.14 g) and potassium carbonate (0.11 g), 2-{[3,5-bis(trifluoromethyl)-1H-1,2,4-triazol-1-yl]methyl}-4-methyl-5-nitropyridine (0.20 g) was obtained.

logP(acid): 3.38

### SYNTHESIS EXAMPLE 69-2

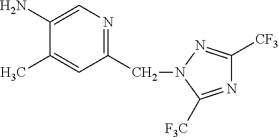

As similar to SYNTHESIS EXAMPLE 1-4, from 2-{[3,5-bis(trifluoromethyl)-1H-1,2,4-triazol-1-yl]methyl}-4-methyl-5-nitro pyridine (0.86 g), tin(II) chloride dihydrate (2.29 g), concentrated hydrochloric acid (5.5 mL) and ethanol (15 mL), 6-{[3,5-bis(trifluoromethyl)-1H-1,2,4-triazol-1-yl]methyl}-4-methylpyridin-3-amine (0.50 g) was obtained.

¹H-NMR (CD₃CN, δ ppm): 2.11 (3H, s), 4.15 (2H, bs), 5.51 (2H, s), 7.00 (1H, s), 7.84 (1H, s).

### SYNTHESIS EXAMPLE 70-1

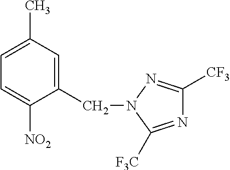

As similar to SYNTHESIS EXAMPLE 8-1, from 5-methyl-2-nitrobenzyl chloride (1.11 g), 3,5-bis(trifluoromethyl)-1H-1,2,4-triazole (1.23 g), potassium carbonate (1.24 g), 18-crown-6 (0.08 g) and tetrabutylammonium iodide (0.11 g), 1-(5-methyl-2-nitrobenzyl)-3,5-bis(trifluoromethyl)-1H-1,2,4-triazole (1.97 g) was obtained.

¹H-NMR (CDCl₃, δ ppm): 2.42 (3H, s), 6.00 (2H, s), 6.63 (1H, s), 7.38 (1H, d), 8.16 (1H, d).

### SYNTHESIS EXAMPLE 70-2

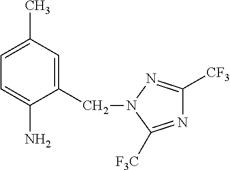

As similar to SYNTHESIS EXAMPLE 1-4, from 1-(5-methyl-2-nitrobenzyl)-3,5-bis(trifluoromethyl)-1H-1,2,4-triazole (1.80 g), tin (II) chloride dihydrate (4.87 g), concentrated hydrochloric acid (5 mL) and ethanol (8 mL), 2-{[3,5-bis(trifluoromethyl)-1H-1,2,4-triazol-1-yl]methyl}-4-methylaniline (1.33 g) was obtained.

¹H-NMR (CDCl₃, δ ppm): 2.24 (3H, s), 4.33 (2H, bs), 5.53 (2H, s), 6.69 (1H, d), 6.98 (1H, s), 7.02 (1H, d).

### SYNTHESIS EXAMPLE 71-1

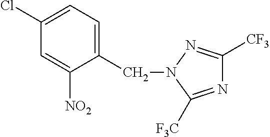

As similar to SYNTHESIS EXAMPLE 8-1, from 4-chloro-2-nitrobenzyl chloride (1.24 g), 3,5-bis(trifluoromethyl)-1H-1,2,4-triazole (1.23 g), potassium carbonate (1.24 g), 18-crown-6 (0.08 g) and tetrabutylammonium iodide (0.11 g), 1-(4-chloro-2-nitrobenzyl)-3,5-bis(trifluoromethyl)-1H-1,2,4-triazole (2.09 g) was obtained.

¹H-NMR (CDCl₃, δ ppm): 5.98 (2H, s), 6.89 (1H, d), 7.66 (1H, dd), 8.24 (1H, d).

### SYNTHESIS EXAMPLE 71-2

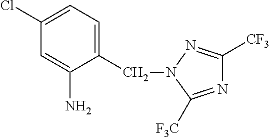

As similar to SYNTHESIS EXAMPLE 1-4, from 1-(4-chloro-2-nitrobenzyl)-3,5-bis(trifluoromethyl)-1H-1,2,4-triazole (2.00 g), tin (II) chloride dihydrate (5.12 g), concentrated hydrochloric acid (5.3 mL) and ethanol (9 mL), 2-{[3,5-bis(trifluoromethyl)-1H-1,2,4-triazol-1-yl]methyl}-4-methyaniline (1.50 g) was obtained.

¹H-NMR (CDCl₃, δ ppm): 4.56 (2H, bs), 5.42 (2H, s), 6.71-6.79 (2H, m), 7.10 (1H, d).

### SYNTHESIS EXAMPLE 72-1

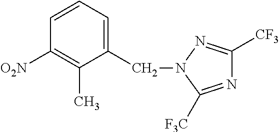

As similar to SYNTHESIS EXAMPLE 8-1, from 2-methyl-3-nitrobenzyl chloride (1.86 g), 3,5-bis(trifluoromethyl)-1H-1,2,4-triazole (2.15 g), potassium carbonate (2.07 g), 18-crown-6 (0.13 g), and tetrabutylammonium iodide (0.18 g), 1-(2-methyl-3-nitrobenzyl)-3,5-bis(trifluoromethyl)-1H-1,2,4-triazole (3.50 g) was obtained.

¹H-NMR (CDCl₃, δ ppm): 2.52 (3H, s), 5.65 (2H, s), 7.17 (1H, d), 7.38 (1H, dd), 7.81 (1H, d).

### SYNTHESIS EXAMPLE 72-2

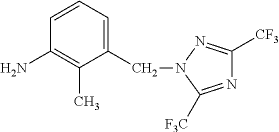

As similar to SYNTHESIS EXAMPLE 1-4, from 1-(2-methyl-3-nitrobenzyl)-3,5-bis(trifluoromethyl)-1H-1,2,4-triazole (3.50 g), tin (II) chloride dihydrate (10.6 g), concentrated hydrochloric acid (11 mL) and ethanol (20 mL), 3-{[3,5-bis(trifluoromethyl)-1H-1,2,4-triazol-1-yl]methyl}-2-methylaniline (3.00 g) was obtained.

¹H-NMR (CDCl₃, δ ppm): 2.18 (3H, s), 3.56-3.84 (2H, m), 5.54 (2H, s), 6.40 (1H, d), 6.71 (1H, d), 7.01 (1H, dd).

### SYNTHESIS EXAMPLE 73-1

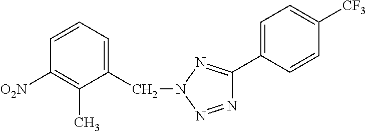

As similar to SYNTHESIS EXAMPLE 1-3, from 2-methyl-3-nitrobenzyl chloride (0.74 g), 5-[4-(trifluoromethyl)phenyl]-1H-tetrazole (0.86 g) and potassium carbonate (0.66 g), 2-(2-methyl-3-nitrobenzyl)-5-[4-(trifluoromethyl)pheny]-2H-tetrazole (1.34 g) was obtained.

¹H-NMR (CDCl₃, δ ppm): 2.61 (3H, s), 5.94 (2H, s), 7.40 (1H, dd), 7.57 (1H, d), 7.74 (2H, d), 7.82 (1H, d), 8.25 (2H, d).

### SYNTHESIS EXAMPLE 73-2

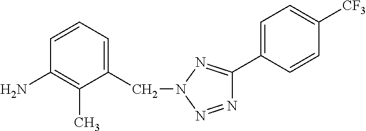

As similar to SYNTHESIS EXAMPLE 1-4, from 2-(2-methyl-3-nitrobenzyl)-5-[4-(trifluoromethyl)phenyl]-2H-tetrazole (1.30 g), tin (II) chloride dihydrate (4.04 g), concentrated hydrochloric acid (4 mL) and ethanol (6 mL), 2-methyl-3-({5-[4-(trifluoromethyl)phenyl]-2H-tetrazol-2-yl}methyl)aniline (1.07 g) was obtained.

¹H-NMR (CDCl₃, δ ppm): 2.25 (3H, s), 3.57-3.79 (2H, m), 5.82 (2H, s), 6.73 (1H, d), 6.81 (1H, d), 7.06 (1H, dd), 7.72 (2H, d), 8.25 (2H, d).

### SYNTHESIS EXAMPLE 74-1

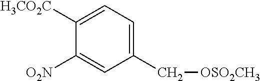

As similar to SYNTHESIS EXAMPLE 68-1, from methyl 4-(hydroxymethyl)-2-nitrobenzoate (1.00 g), triethylamine (1 mL) and methanesulfonyl chloride (0.60 g), methyl 4-{[(methylsulfonyl)oxy]methyl}-2-nitrobenzoate (1.35 g) was obtained.

¹H-NMR (CDCl₃, δ ppm): 3.09 (3H, s), 3.95 (3H, s), 5.32 (2H, s), 7.71 (1H, d), 7.80 (1H, d), 7.94 (1H, s).

### SYNTHESIS EXAMPLE 74-2

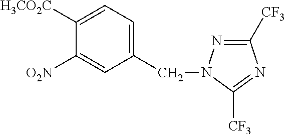

Methyl 4-{[(methylsulfonyl)oxy]methyl}-2-nitrobenzoate (1.81 g), 3,5-bis(trifluoromethyl)-1H-1,2,4-triazole (0.66 g) and potassium carbonate (0.49 g) were heated to reflux in acetonitrile (50 mL) for 3 hours. After the reaction was completed, water was added, and the mixture was extracted with ethyl acetate. The organic phase was washed with saturated brine, and dried over anhydrous magnesium sulfate. The solvent was distilled off, and the resulting crude product was purified with silica gel column chromatography to obtain methyl 4-{[3,5-bis(trifluoromethyl)-1H-1,2,4-triazol-1-yl]methyl}-2-nitrobenzoate (0.80 g).

¹H-NMR (CDCl₃, δ ppm): 3.92 (3H, s), 5.62 (2H, s), 7.62 (1H, d), 7.80 (1H, d), 7.90 (1H, s).

### SYNTHESIS EXAMPLE 74-3

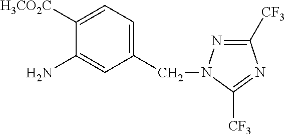

As similar to SYNTHESIS EXAMPLE 1-4, from methyl 4-{[3,5-bis(trifluoromethyl)-1H-1,2,4-triazol-1-yl]methyl}-2-nitrobenzoate (0.25 g), tin (II) chloride dihydrate (0.71 g), concentrated hydrochloric acid (0.6 mL) and ethanol (1 mL), methyl 2-amino-4-{[3,5-bis(trifluoromethyl)-1H-1,2,4-triazol-1-yl]methyl}benzoate (0.25 g) was obtained.

¹H-NMR (CDCl₃, δ ppm): 3.87 (3H, s), 5.42 (2H, s), 5.82 (2H, br s), 6.51 (2H, d), 7.87 (1H, d).

### SYNTHESIS EXAMPLE 75-1

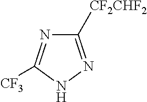

To of methanol solution (43 mL) of hydrazine hydrate (1.35 g), 2,2,3,3-tetrafluoropropionic acid methyl ester (4.80 g) was added dropwise at a temperature not exceeding 5° C. over 30 minutes, and subsequently, the mixture was stirred at 0° C. for 1 hour. The solvent was distilled off, and the resulting residue was dissolved in THF (70 mL), trifluoro acetoamidine (3.36 g) was added, and the mixture was heated to reflux for 3 hours. After cooled to room temperature, the solvent was distilled off, and the resulting residue was dissolved in toluene (64 mL). Trifluoroacetic acid (0.34 g) was added, and the mixture was heated to reflux for 4 hours. After cooled to room temperature, the solvent was distilled off to obtain crude 3-(1,1,2,2-tetrafluoroethyl)-5-(trifluoromethyl)-1H-1,2,4-triazole (7.68 g).

¹H-NMR (CDCl₃, δ ppm): 4.02-4.70 (1H, m), 6.25 (1H, tt).

### SYNTHESIS EXAMPLE 75-2

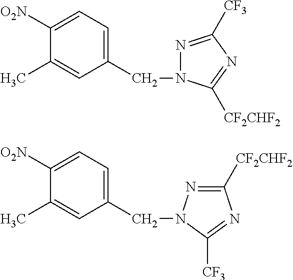

3-methyl-4-nitrobenzylchloride (2.78 g), 3-(1,1,2,2-tetrafluoroethyl)-5-(trifluoromethyl)-1H-1,2,4-triazole (4.89 g), potassium carbonate (3.11 g), 18-crown-6 (0.20 g) and tetrabutylammonium iodide (0.28 g) were heated to reflux in propionitrile (38 mL) for 2 hours. After cooling to room temperature, water (100 mL) was added to the reaction mixture, which was then extracted with ethyl acetate (100 mL). The organic phase was washed subsequently with 5% sodium hydroxide aqueous solution, 0.5N hydrochloric acid and saturated brine, and dried over anhydrous sodium sulfate. The solvent was distilled off under reduced pressure, and the resulting crude product was purified with silica gel column chromatography (mixed solvent of n-hexane and ethyl acetate) to obtain, as the first elution portion, 1-(1-(3-methyl-4-nitrobenzyl)-5-(1,1,2,2-tetrafluoroethyl)-3-trifluoromethyl-1H-[1,2,4]-triazole (1.50 g);

¹H-NMR (CDCl₃, δ ppm): 2.62 (3H, s), 5.60 (2H, s), 6.40 (1H, tt), 7.24-7.33 (2H, m), 7.99 (1H, d);

and, as the second elution portion, 1-(1-(3-methyl-4-nitrobenzyl)-3-(1,1,2,2-tetrafluoroethyl)-5-trifluoromethyl-1H-[1,2,4]-triazole (0.60 g).

¹H-NMR (CDCl₃, δ ppm): 2.60 (3H, s), 5.59 (2H, s), 6.21 (1H, tt), 7.22-7.30 (2H, m), 7.99 (1H, d).

### SYNTHESIS EXAMPLE 75-3

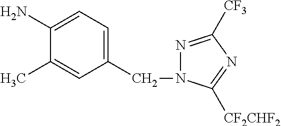

As similar to SYNTHESIS EXAMPLE 1-4, from 1-(1-(3-methyl-4-nitrobenzyl)-5-(1,1,2,2-tetrafluoroethyl)-3-trifluoromethyl-1H-[1,2,4]-triazole (0.60 g), tin (II) chloride dihydrate (1.75 g), concentrated hydrochloric acid (2 mL) and ethanol (3 mL), 2-methyl-4-{([5-(1,1,2,2-tetrafluoroethyl)-3-(trifluoromethyl)-1H-1,2,4-triazol-1-yl]methyl}aniline (0.42 g) was obtained.

¹H-NMR (CDCl₃, δ ppm): 2.15 (3H, s), 3.61-3.82 (2H, m), 5.42 (2H, s), 6.36 (1H, tt), 6.64 (1H, d), 7.02-7.10 (2H, m).

### SYNTHESIS EXAMPLE 75-4

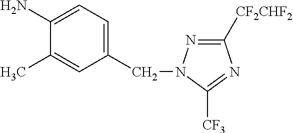

As similar to SYNTHESIS EXAMPLE 1-4, from 1-(1-(3-methyl-4-nitrobenzyl)-5-(1,1,2,2-tetrafluoroethyl)-3-trifluoromethyl-1H-[1,2,4]-triazole (0.60 g), tin (II) chloride dihydrate (1.75 g), concentrated hydrochloric acid (2 mL) and ethanol (3 mL), 2-methyl-4-{[3-(1,1,2,2-tetrafluoroethyl)-5-(trifluoromethyl)-1H-1,2,4-triazol-1-yl]methyl}aniline (0.50 g) was obtained.

¹H-NMR (CDCl₃, δ ppm): 2.15 (3H, s), 3.35-3.95 (2H, m), 5.38 (2H, s), 6.21 (1H, tt), 6.63 (1H, d), 6.98-7.06 (2H, m).

### SYNTHESIS EXAMPLE 76-1

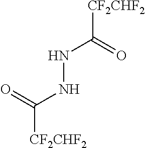

To a toluene solution (500 mL) of 2,2,3,3-tetrafluoropropionic acid (25.0 g), hydrazine hydrate (8.1 g) was added, and then the mixture was heated to reflux for 2 hours while dehydrating. Further, 2,2,3,3-tetrafluoropropionic acid (25.0 g) was added, and the mixture was heated to reflux for 16 hours while dehydrating. After an insoluble matter was filtered off in the hot state, the filtrate was allowed to stand at room temperature overnight, and the precipitated crude crystal was washed with toluene to obtain crude 2,2,3,3-tetrafluoro-N′-(2,2,3,3-tetrafluoropropanoyl)-propanehydrazide (60.0 g), which was subjected to the next reaction without purification.

### SYNTHESIS EXAMPLE 76-2

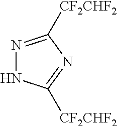

A mixture of N,N-dimethylaniline hydrochloride (53 g), phosphorus oxychloride (76 g) and 2,3,3-tetrafluoro-N′-(2,2,3,3-tetrafluoropropanoyl)propane hydrazide (46 g) was heated to reflux for 3 hours and cooled to 0° C. Water (400 mL) was carefully added thereto, and the mixture was extracted with diethyl ether for several times. The organic phase was washed sequentially with 2N hydrochloric acid and saturated brine, and dried over anhydrous sodium sulfate. After the solvent was distilled off, crude N-(1-chloro-2,2,3,3-tetrafluoropropylidene)-2,2,3,3-tetrafluoropropanehydrazonoyl chloride (30 g) was obtained. This crude product was dissolved in diethyl ether solution (40 mL) again, and cooled to 0° C. Subsequently, 5% ammonia water (25 g) was added dropwise at 0° C. over 10 minutes, followed by stirring for 5 minutes. The organic phase was separated, washed with saturated brine and dried over anhydrous sodium sulfate. After distilling off the solvent, the resulting crude N-(1-amino-2,2,3,3-tetrafluoropropylidene)-2,2,3,3-tetrafluoro-propanehydrazonoyl chloride (31 g) was dissolved in acetonitrile (500 mL), and potassium carbonate (28 g) was added. The mixture was heated and stirred at 50° C. for 4 hours. After the reaction was completed, water and diethyl ether were added, and the aqueous phase was separated. After washed with diethyl ether, the aqueous phase was acidified with concentrated hydrochloric acid, extracted with diethyl ether and dried over anhydrous sodium sulfate. The solvent was distilled off to obtain 3,5-bis(1,1,2,2-tetrafluoroethyl)-1H-1,2,4-triazole (7.0 g).

¹H-NMR (CDCl₃, δ ppm): 5.29-5.89 (1H, m), 6.24 (2H, tt).

### SYNTHESIS EXAMPLE 76-3

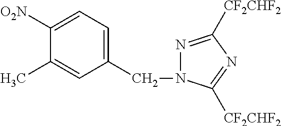

As similar to SYNTHESIS EXAMPLE 8-1, from 2-methyl-4-nitrobenzylchloride (3.72 g), 3,5-bis(1,1,2,2-tetrafluoroethyl)-1H-1,2,4-triazole (5.39 g), potassium carbonate (4.15 g), 18-crown-6 (0.27 g) and tetrabutylammonium iodide (0.37 g), 1-(2-methyl-4-nitrobenzyl)-3,5-bis(1,1,2,2-tetrafluoroethyl)-1H-1,2,4-triazole (4.13 g) was obtained.

¹H-NMR (CDCl₃, δ ppm): 2.60 (3H, s), 5.61 (2H, s), 6.20 (1H, tt), 6.39 (1H, tt), 7.22-7.31 (2H, m), 7.99 (1H, d).

### SYNTHESIS EXAMPLE 76-4

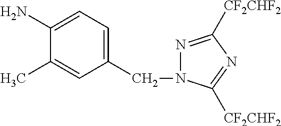

As similar to SYNTHESIS EXAMPLE 1-4, from 1-(2-methyl-4-nitrobenzyl)-3,5-bis(1,1,2,2-tetrafluoroethyl)-1H-1,2,4-triazole (3.5 g), tin (II) chloride dihydrate (9.44 g), concentrated hydrochloric acid (10 mL) and ethanol (15 mL), 4-{[3,5-bis(1,1,2,2-tetrafluoro ethyl)-1H-1,2,4-triazol-1-yl]methyl}-2-methylaniline (2.78 g) was obtained.

¹H-NMR (CDCl₃, δ ppm): 2.15 (3H, s), 3.70 (2H, bs), 5.42 (2H, s), 6.20 (1H, tt), 6.34 (1H, tt), 6.63 (1H, d), 7.00-7.08 (2H, m).

### SYNTHESIS EXAMPLE 77-1

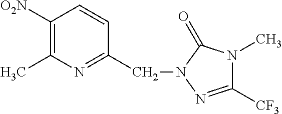

As similar to SYNTHESIS EXAMPLE 1-3, from 6-(bromomethyl)-2-methyl-3-nitropyridine (2.00 g), 4-methyl-5-(trifluoromethyl)-2,4-dihydro-3H-1,2,4-triazol-3-one (1.30 g) and potassium carbonate (1.18 g), 4-methyl-2-[(6-methyl-5-nitropyridine-2-yl)methyl]-5-(trifluoromethyl)-2,4-dihydro-3H-1,2,4-triazol-3-one (1.50 g) was obtained.

¹H-NMR (DMSO-d₆, δ ppm): 2.70 (3H, s), 3.34 (3H, s), 5.19 (2H, s), 7.40 (1H, d), 8.38 (1H, d).

### SYNTHESIS EXAMPLE 77-2

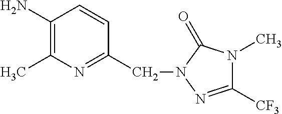

As similar to SYNTHESIS EXAMPLE 1-4, from 4-methyl-2-[(6-methyl-5-nitropyridin-2-yl)methyl]-5-(trifluoromethyl)-2,4-dihydro-3H-1,2,4-triazol-3-one (1.20 g), tin (II) chloride dihydrate (4.14 g), concentrated hydrochloric acid (20 mL) and ethanol (30 mL), 2-[(5-amino-6-methylpyridin-2-yl)methyl]-4-methyl-5-(trifluoromethyl)-2,4-dihydro-3H-1,2,4-triazol-3-one (0.95 g) was obtained.

¹H-NMR (DMSO-d₆, δ ppm): 2.23 (3H, s), 3.31 (3H, s), 4.84 (2H, s), 4.85-4.92 (2H, m), 6.81 (1H, d), 6.90 (1H, d).

### SYNTHESIS EXAMPLE 78-1

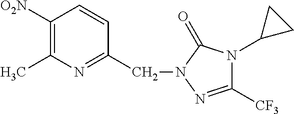

As similar to SYNTHESIS EXAMPLE 1-3, from 6-(bromomethyl)-2-methyl-3-nitropyridine (1.42 g), 4-cyclopropyl-5-(trifluoromethyl)-2,4-dihydro-3H-1,2,4-triazol-3-one (1.20 g) and potassium carbonate (0.95 g), 4-cyclopropyl-2-[(6-methyl-5-nitropyridin-2-yl)methyl]-5-(trifluoromethyl)-2,4-dihydro-3H-1,2,4-triazol-3-one (1.35 g) was obtained.

¹H-NMR (DMSO-d₆, δ ppm): 0.96-1.09 (4H, m), 2.70 (3H, s), 3.00-3.09 (1H, m), 5.12 (2H, s), 7.39 (1H, d), 8.38 (1H, d).

### SYNTHESIS EXAMPLE 78-2

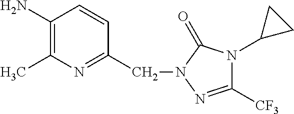

As similar to SYNTHESIS EXAMPLE 1-4, from 4-cyclopropyl-2-[(6-methyl-5-nitropyridin-2-yl)methyl]-5-(trifluoromethyl)-2,4-dihydro-3H-1,2,4-triazol-3-one (1.10 g), tin (II) chloride dihydrate (3.32 g), concentrated hydrochloric acid (15 mL) and ethanol (25 mL), 2-[(5-amino-6-methylpyridin-2-yl)methyl]-4-cyclopropyl-5-(trifluoro-methyl)-2,4-dihydro-3H-1,2,4-triazol-3-one (0.75 g) was obtained.

¹H-NMR (DMSO-d₆, δ ppm): 0.95-1.08 (4H, m), 2.23 (3H, s), 2.96-3.03 (1H, m), 4.68 (2H, s), 4.87 (2H, bs), 6.80 (1H, d), 6.90 (1H, d).

### SYNTHESIS EXAMPLE 79-1

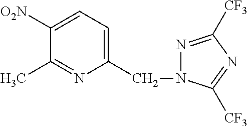

As similar to SYNTHESIS EXAMPLE 1-3, from 6-(bromomethyl)-2-methyl-3-nitropyridine (0.59 g), 3,5-bis(trifluoromethyl)-1H-1,2,4-triazole (0.52 g) and potassium carbonate (0.88 g), 6-{[3,5-bis(trifluoromethyl)-1H-1,2,4-triazol-1-yl]methyl)-2-methyl-3-nitropyridine (1.00 g) was obtained.

¹H-NMR (DMSO-d₆, δ ppm): 2.61 (3H, s), 5.96 (2H, s), 7.65 (1H, d), 8.42 (1H, d).

### SYNTHESIS EXAMPLE 79-2

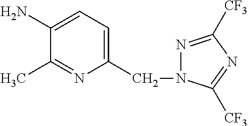

As similar to SYNTHESIS EXAMPLE 1-4, from 6-{[3,5-bis(trifluoromethyl)-1H-1,2,4-triazol-1-yl]methyl}-2-methyl-3-nitropyridine (1.40 g), tin (II) chloride dihydrate (4.45 g), concentrated hydrochloric acid (8 mL) and ethanol (15 mL), 6-{[3,5-bis(trifluoromethyl)-1H-1,2,4-triazol-1-yl]methyl}-2-methylpyridin-3-amine (0.70 g) was obtained.

¹H-NMR (DMSO-d₆, δ ppm): 2.18 (3H, s), 4.70-5.30 (2H, m), 5.57 (2H, s), 6.90-7.00 (2H, m).

### SYNTHESIS EXAMPLE 80-1

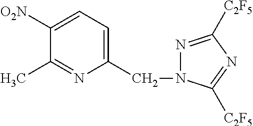

As similar to SYNTHESIS EXAMPLE 1-3, from 6-(bromomethyl)-2-methyl-3-nitropyridine (1.54 g), 3,5-bis(pentafluoroethyl)-1H-1,2,4-triazole (3.00 g) and potassium carbonate (0.95 g), 6-{[3,5-bis(pentafluoroethyl)-1H-1,2,4-triazol-1-yl]methyl}-2-methyl-3-nitropyridine (2.90 g) was obtained.

¹H-NMR (DMSO-d₆, δ ppm): 2.55 (3H, s), 6.08 (2H, s), 7.51 (1H, d), 8.48 (1H, d).

### SYNTHESIS EXAMPLE 80-2

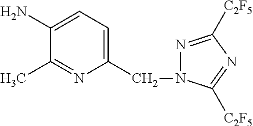

As similar to SYNTHESIS EXAMPLE 1-4, from 6-{[3,5-bis(pentafluoroethyl)-1H-1,2,4-triazol-1-yl]methyl}-2-methyl-3-nitropyridine (2.90 g), tin (II) chloride dihydrate (7.55 g), concentrated hydrochloric acid (12 mL) and ethanol (20 mL), 6-{[3,5-bis(pentafluoroethyl)-1H-1,2,4-triazol-1-yl]methyl}-2-methylpyridin-3-amine (0.75 g) was obtained.

¹H-NMR (CDCl₃, δ ppm): 2.32 (3H, s), 3.30-4.00 (2H, m), 5.58 (2H, s), 6.82 (1H, d), 6.89 (1H, d).

### SYNTHESIS EXAMPLE 81-1

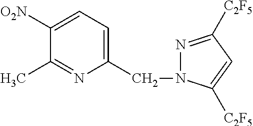

As similar to SYNTHESIS EXAMPLE 1-3, from 6-(bromomethyl)-2-methyl-3-nitropyridine (1.00 g), 3,5-bis(pentafluoroethyl)-1H-pyrazole (1.00 g) and potassium carbonate (0.65 g), 6-{[3,5-bis(pentafluoroethyl)-1H-pyrazol-1-yl]methyl}-2-methyl-3-nitropyridine (1.80 g) was obtained.

logP(acid): 4.68

### SYNTHESIS EXAMPLE 81-2

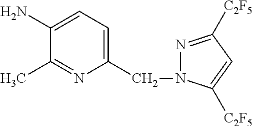

As similar to SYNTHESIS EXAMPLE 1-4, from 6-{[3,5-bis(pentafluoroethyl)-1H-pyrazol-1-yl]methyl)-2-methyl-3-nitropyridine (1.80 g), tin (II) chloride dihydrate (4.76 g), concentrated hydrochloric acid (12 mL) and ethanol (15 mL), 6-{[3,5-bis(pentafluoroethyl)-1H-pyrazol-1-yl]methyl}-2-methylpyridin-3-amine (1.19 g) was obtained.

logP(acid): 2.83

### SYNTHESIS EXAMPLE 82-1

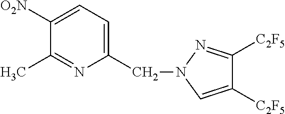

As similar to SYNTHESIS EXAMPLE 1-3, from 6-(bromomethyl)-2-methyl-3-nitropyridine (0.87 g), 3,4-bis(pentafluoroethyl)-1H-pyrazole (1.10 g) and potassium carbonate (0.57 g), 6-{[3,4-bis(pentafluoroethyl)-1H-pyrazol-1-yl]methyl}-2-methyl-3-nitropyridine (1.75 g) was obtained.

logP(acid): 4.50.

### SYNTHESIS EXAMPLE 82-2

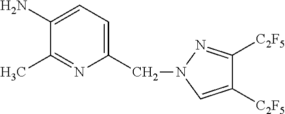

As similar to SYNTHESIS EXAMPLE 1-4, from 6-{[3,4-bis(pentafluoromethyl)-1H-pyrazol-1-yl]methyl}-2-methyl-3-nitropyridine (1.70 g), tin (II) chloride dihydrate (4.14 g), concentrated hydrochloric acid (12 mL) and ethanol (15 mL), 6-{[3,4-bis(pentafluoroethyl)-1H-pyrazol-1-yl]methyl}-2-methylpyridin-3-amine (1.10 g) was obtained.

logP(acid): 2.64.

### SYNTHESIS EXAMPLE 83-1

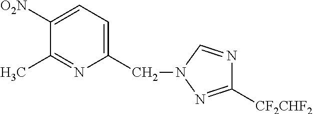

As similar to SYNTHESIS EXAMPLE 1-3, from 6-(bromomethyl)-2-methyl-3-nitropyridine (2.50 g), 3-(1,1,2,2-tetrafluoro ethyl)-1H-1,2,4-triazole (1.83 g) and potassium carbonate (1.94 g), 2-methyl-3-nitro-6-{[3-(1,1,2,2-tetrafluoroethyl)-1H-1,2,4-triazol-2-yl]methyl}pyridine (2.10 g) was obtained.

logP(acid): 2.26.

### SYNTHESIS EXAMPLE 83-2

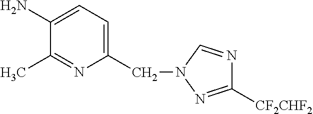

As similar to SYNTHESIS EXAMPLE 1-4, from 2-methyl-3-nitro-6-{[3-(1,1,2,2-tetrafluoroethyl)-1H-1,2,4-triazol-2-yl]methyl}pyridine (1.70 g), tin (II) chloride dihydrate (5.04 g), concentrated hydrochloric acid (12 mL) and ethanol (32 mL), 2-methyl-6-{[3-(1,1,2,2-tetrafluoroethyl)-1H-1,2,4-triazol-1-yl]methyl}pyridin-3-amine (1.50 g) was obtained.

logP(acid): 0.55.

### SYNTHESIS EXAMPLE 84-1

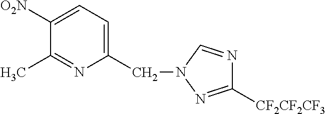

As similar to SYNTHESIS EXAMPLE 1-3, from 6-(bromomethyl)-2-methyl-3-nitropyridine (2.00 g), 3-(heptafluoropropyl)-1H-1,2,4-triazole (2.05 g) and potassium carbonate (1.55 g), 2-methyl-3-nitro-6-{[3-(heptafluoropropyl)-1H-1,2,4-triazol-2-yl]methyl}pyridine (1.50 g) was obtained.

logP(acid): 3.23

### SYNTHESIS EXAMPLE 84-2

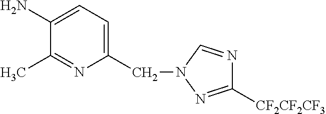

As similar to SYNTHESIS EXAMPLE 1-4, from 2-methyl-3-nitro-6-{[3-(heptafluoropropyl)-1H-1,2,4-triazol-2-yl]methyl}pyridine (1.50 g), tin (II) chloride dihydrate (3.67 g), concentrated hydrochloric acid (9 mL) and ethanol (24 mL), 6-{[3-(heptafluoropropyl)-1H-1,2,4-triazol-1-yl]methyl}-2-methylpyridin-3-amine (1.20 g) was obtained.

logP(acid): 1.52

### SYNTHESIS EXAMPLE 85-1

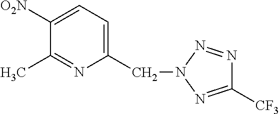

As similar to SYNTHESIS EXAMPLE 1-3, from 6-(bromomethyl)-2-methyl-3-nitropyridine (3.35 g), 5-(trifluoro methyl)-2H-tetrazole (2.00 g) and potassium carbonate (2.60 g), 2-methyl-3-nito-6-{[5-(trifluoromethyl)-2H-tetrazol-2-yl]methyl}pyridine (1.70 g) was obtained.

logP(acid): 2.85.

### SYNTHESIS EXAMPLE 85-2

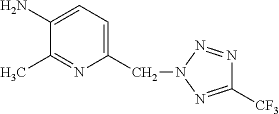

As similar to SYNTHESIS EXAMPLE 1-4, from 2-methyl-3-nitro-6-{[5-(trifluoromethyl)-2H-tetrazol-2-yl]methyl}pyridine (1.50 g), tin (II) chloride dihydrate (4.90 g), concentrated hydrochloric acid (12 mL) and ethanol (32 mL), 2-methyl-6-{[5-(trifluoromethyl)-2H-tetrazol-2-yl]methyl}pyridin-3-amine (1.10 g) was obtained.

logP(acid): 1.18

### SYNTHESIS EXAMPLE 86-1

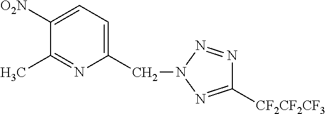

As similar to SYNTHESIS EXAMPLE 1-3, from 6-(bromomethyl)-2-methyl-3-nitropyridine (2.00 g), 5-(heptafluoropropyl)-2H-tetrazole (2.06 g) and potassium carbonate (1.55 g), 6-{[5-(heptafluoropropyl)-2H-tetrazol-2-yl]methyl}-2-methyl-3-nitro-pyridine (1.15 g) was obtained.

logP(acid): 3.78

### SYNTHESIS EXAMPLE 86-2

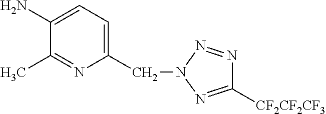

As similar to SYNTHESIS EXAMPLE 1-4, from 6-{[5-(heptafluoropropyl)-2H-tetrazol-2-yl]methyl}-2-methyl-3-nitro-pyridine (1.00 g), tin (II) chloride dihydrate (2.43 g), concentrated hydrochloric acid (5 mL) and ethanol (15 mL), 6-{[5-(heptafluoropropyl)-2H-tetrazol-2-yl]methyl}-2-methylpyridin-3-amine (0.85 g) was obtained.

logP(acid): 2.3

### SYNTHESIS EXAMPLE 87-1

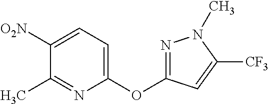

Under argon atmosphere, 60% sodium hydride (0.27 g) was added to DMF (20 mL), and then a DMF solution (10 mL) of (3-hydroxy-1-methyl-5-(trifluoromethyl)pyrazole (1.03 g) was added dropwise at room temperature over 30 minutes. Further, 6-chloro-2-methyl-3-nitropyridine (1.07 g) was added, and the mixture stirred at room temperature for 16 hours. Water was added to the reaction mixture, which was then extracted with t-butylmethylether. The organic phase was washed sequentially with water and saturated brine and was dried over anhydrous sodium sulfate. After the solvent was distilled off, the crude product was purified with silica gel column chromatography (mixed solvent of cyclohexane and ethyl acetate) to obtain 2-methyl-6-{[1-methyl-5-(trifluoromethyl)-1H-pyrazol-3-yl]oxy}-3-nitropyridine (1.43 g).

¹H-NMR (DMSO-d₆, δ ppm): 2.65 (3H, s), 3.92 (3H, s), 6.82 (1H, s), 7.16 (1H, d), 8.49 (1H, d).

### SYNTHESIS EXAMPLE 87-2

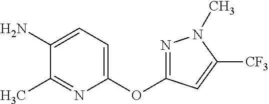

Similar to SYNTHESIS EXAMPLE 1-4, from 2-methyl-6-{[1-methyl-5-(trifluoromethyl)-1H-pyrazol-3-yl]oxy}-3-nitropyridine (1.34 g), tin (II) chloride dihydrate (5.02 g), concentrated hydrochloric acid (15 mL) and ethanol (20 mL), 2-methyl-6-{[1-methyl-5-(trifluoromethyl)-1H-pyrazol-3-yl]oxy}pyridin-3-amine (1.17 g) was obtained.

¹H-NMR (DMSO-d₆, δ ppm): 2.18 (3H, s), 3.83 (3H, s), 4.66-4.75 (2H, m), 6.43 (1H, s), 6.66 (1H, d), 7.07 (1H, d).

### SYNTHESIS EXAMPLE 88-1

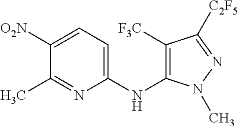

1-Methyl-3-(pentafluoroethyl)-4-(trifluoromethyl)-1H-pyrazol-5-amine (4.90 g) and cesium carbonate (11.82 g) were stirred in acetonitrile (160 mL) at room temperature for 15 minutes. Subsequently, 6-chloro-2-methyl-3-nitropyridine (1.07 g) was added, and the mixture was heated to reflux for 1 hour. After the reaction was completed and cooled to room temperature, an insoluble matter was filtered off. The filtrate was distilled off, and the resulting crude product was purified with a silica gel column chromatography (mixed solvent of dichloromethane and ethyl acetate) to obtain 6-methyl-N-[1-methyl-3-(pentafluoroethyl)-4-(trifluoromethyl)-1H-pyrazol-5-yl]-5-nitropyridin-2-amine (1.40 g).

¹H-NMR (DMSO-d₆, δ ppm): 2.02 (1H, s), 2.57 (3H, s), 3.77 (3H, s), 6.81 (1H, s), 8.30 (1H, d).

### SYNTHESIS EXAMPLE 88-2

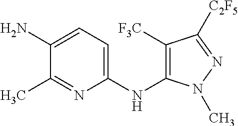

As similar to SYNTHESIS EXAMPLE 1-4, from 6-methyl-N-[1-methyl-3-(pentafluoroethyl)-4-(trifluoromethyl)-1H-pyrazol-5-yl]-5-nitropyridin-2-amine (0.55 g), tin (II) chloride dihydrate (1.54 g), concentrated hydrochloric acid (6 mL), ethanol (11 mL), 6-methyl-N²-[1-methyl-3-(pentafluoroethyl)-4-(trifluoromethyl)-1H-pyrazol-5-yl]pyridine-2,5-diamine (0.29 g) was obtained.

¹H-NMR (DMSO-d₆, δ ppm): 2.10 (3H, s), 3.38 (3H, s), 4.31 (2H, bs), 6.41 (1H, d), 6.94 (1H, d), 8.21 (1H, bs).

### SYNTHESIS EXAMPLE 88-3

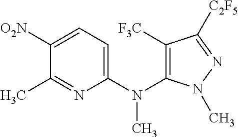

Under argon atmosphere, to a THF solution (20 mL) of 6-methyl-N-[1-methyl-3-(pentafluoroethyl)-4-(trifluoromethyl)-1H-pyrazol-5-yl]-5-nitropyridin-2-amine (0.60 g), 60% sodium hydride (0.06 g) was added, and the mixture was stirred at room temperature for 45 minutes. Subsequently, methyliodide (0.40 g) was added, and this was stirred at room temperature for another 2 hours. Water was added to the reaction mixture, and the mixture was extracted with ethylacetate. The organic phase was washed with saturated brine and dried over anhydrous sodium sulfate. The solvent was distilled off, and the crude product was purified with silica gelcolunm chromatography (mixed solvent of cyclohexane and ethyl acetate) to obtain N,6-dimethyl-N-[1-methyl-3-(pentafluoroethyl)-4-(trifluoromethyl)-1H-pyrazol-5-yl]-5-nitropyridin-2-amine (0.32 g).

¹H-NMR (DMSO-d₆, δ ppm): 2.60 (3H, s), 3.42 (3H, s), 3.76 (3H, s), 6.72 (1H, s), 8.35 (1H, d).

### SYNTHESIS EXAMPLE 88-4

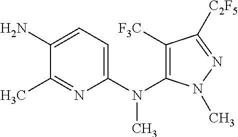

As similar to SYNTHESIS EXAMPLE 1-4, from N,6-dimethyl-N-[1-methyl-3-(pentafluoroethyl)-4-(trifluoromethyl)-1H-pyrazol-5-yl]-5-nitropyridin-2-amine (0.29 g), tin (II) chloride dihydrate (0.71 g), concentrated hydrochloric acid (3 mL) and ethanol (5 mL), N²,6-dimethyl-N²-[1-methyl-3-(pentafluoroethyl)-4-(trifluoromethyl)-1H-pyrazol-5-yl]pyridine-2,5-diamine (0.26 g) was obtained.

¹H-NMR (DMSO-d₆, δ ppm): 2.12 (3H, s), 3.19 (3H, s), 3.67 (3H, s), 4.30-4.40 (2H, m), 6.22 (1H, d), 6.96 (1H, d).

### SYNTHESIS EXAMPLE 89-1

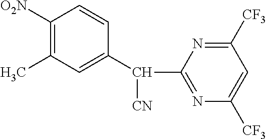

Under argon atmosphere, 60% sodium hydride (0.20 g) was added to DMF (6 mL), and subsequently a DMF solution (2 mL) of (3-methyl-4-nitrophenyl)acetonitrile (0.60 g) was added dropwise at room temperature. Further, a DMF solution (2 mL) of 2-methanesulfonyl-4,6-bis(trifluoromethyl)pyrimidine (1.00 g) was added, and the mixture was stirred at room temperature for 1 hour. Water was added to the reaction mixture, and after acidified with 2N hydrochloric acid, the reaction mixture was extracted with ethyl acetate and dried over anhydrous magnesium sulfate. After the solvent was distilled off, the crude product was purified with silica gel column chromatography (mixed solvent of n-hexane and ethyl acetate) to obtain [4,6-bis(trifluoromethyl)pyrimidine-2-yl](3-methyl-4-nitrophenyl)acetonitrile (0.95 g).

¹H-NMR (CDCl₃, δ ppm): 2.63 (3H, s), 5.64 (1H, s), 7.58-7.64 (2H, m), 7.95 (1H, s), 8.00 (1H, d).

### SYNTHESIS EXAMPLE 89-2

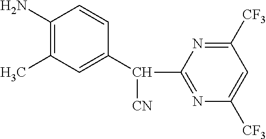

As similar to SYNTHESIS EXAMPLE 17-2, from [4,6-bis(trifluoromethyl)pyrimidin-2-yl](3-methyl-4-nitrophenyl)acetonitrile (0.30 g), ammonium acetate (5.92 g), acetone (15 mL), water (15 mL), and 20% titanium trichloride aqueous solution (5.34 g), (4-amino-3-methylphenyl)[4,6-bis(trifluoromethyl)pyrimidin-2-yl]acetonitrile (0.28 g) was obtained.

¹H-NMR (CDCl₃, δ ppm): 2.17 (3H, s), 3.59-3.90 (2H, m), 5.45 (1H, s), 6.65 (1H, s), 7.18-7.27 (2H, m), 7.85 (1H, s).

### SYNTHESIS EXAMPLE 89-3

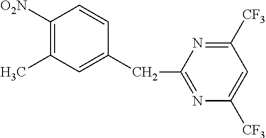

A mixture of [4,6-bis(trifluoromethyl)pyrimidine-2-yl](3-methyl-4-nitrophenyl)acetonitrile (0.55 g), concentrated sulfuric acid (1 mL), acetic acid (1 mL) and water (1 mL) was heated to reflux for 6 hours. After cooled to room temperature, the reaction mixture was poured into ice water. After alkalized with sodium hydride carbonate, the reaction mixture was extracted with t-butylmethylether and dried over anhydrous magnesium sulfate, and the solvent was distilled off to obtain 2-(3-methyl-4-nitrobenzyl)-4,6-bis(trifluoromethyl)pyrimidine (0.42 g).

¹H-NMR (CDCl₃, δ ppm): 2.60 (3H, s), 4.49 (1H, s), 7.35-7.45 (2H, m), 7.82 (1H, s), 7.95 (1H, d).

### SYNTHESIS EXAMPLE 89-4

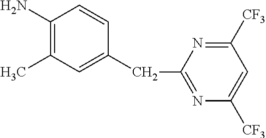

As similar to SYNTHESIS EXAMPLE 17-2, from 2-(3-methyl-4-nitrobenzyl)-4,6-bis(trifluoromethyl)pyrimidine (0.42 g), ammonium acetate (8.86 g), acetone (20 mL), water (20 mL) and 20% titanium trichloride aqueous solution (7.98 g), 4-{[4,6-bis(trifluoromethyl)pyrimidin-2-yl]methyl}-2-methylaniline (0.39 g) was obtained.

¹H-NMR (CDCl₃, δ ppm): 2.14 (3H, s), 3.34-3.89 (2H, m), 4.30 (2H, s), 6.62 (1H, d), 7.05-7.13 (2H, m), 7.73 (1H, s).

### SYNTHESIS EXAMPLE 90-1

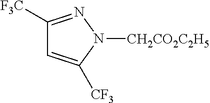

A mixture of 3,5-bis(trifluoromethyl)-1H-pyrazole (1.84 g), bromoethyl acetate (2.04 g) and potassium carbonate (1.66 g) was heated and stirred at 60 to 80° C. in DMF (15 mL) for 1 hour. After the reaction was completed, an insoluble matter was filtered off with Celite. The filtrate was distilled off, and water (100 mL) was added to the resulting crude product, which was then extracted with ethyl acetate. After the organic phase was washed with saturated brine (100 mL), the crude product was purified with silica gel column chromatography (toluene solvent) to obtain [3,5-bis(trifluoromethyl)-1H-pyrazol-1-yl]ethyl acetate ester (2.16 g).

¹H-NMR (CDCl₃, δ ppm): 1.28 (3H, t), 4.26 (2H, q), 5.08 (2H, s), 6.96 (1H, s).

### SYNTHESIS EXAMPLE 90-2

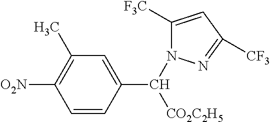

To a DMF suspension (6 mL) of 60% oil based sodium hydride (0.10 g),2-[3,5-bis(trifluoromethyl)-1H-pyrazol-1-yl]ethyl acetate ester (0.87 g) was added at room temperature. After hydrogen generation ceased, 5-fluoro-2-nitrotoluene (0.47 g) was added, and the mixture was stirred at room temperature for 1 hour. The reaction mixture was poured into water, and after acidified with 2N hydrochloric acid aqueous solution, the mixture was extracted with ethyl acetate.

After the organic phase was washed with water and dried with sodium sulfuric anhydrate, the solvent was distilled off to obtain crude [3,5-bis(trifluoromethyl)-1H-pyrazol-1-yl](3-methyl-4-nitrophenyl)ethyl acetate ester (1.5 g, purity approximately 50%) as oily matter, which was subjected to the next reaction without purification.

### SYNTHESIS EXAMPLE 90-3

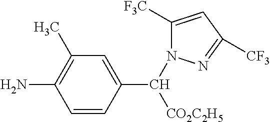

To a mixture of crude [3,5-bis(trifluoromethyl)-1H-pyrazol-1-yl](3-methyl-4-nitrophenyl)ethyl acetate ester (1.5 g, approximate purity 50%), ammonium acetate (13.6 g), acetone (30 mL) and water (14 mL), 20% titanium trichloride aqueous solution (12.2 g) was added, and the mixture was stirred at room temperature for 12 hours. After completion of reaction, the mixture was extracted with ethyl acetate, washed with saturated brine, and dried over anhydrous magnesium sulfate, and the solvent was distilled off to obtain 4-{1-[3,5-bis(trifluoromethyl)-1H-pyrazol-1-yl]ethyl}-2-methylaniline (0.6 g).

¹H-NMR (CDCl₃, δ ppm): 1.27 (3H, t), 2.16 (3H, s), 4.26 (2H, t), 4.70-4.99 (2H, m), 6.02 (1H, s), 6.64-7.18 (4H, m).

### SYNTHESIS EXAMPLE 91-1

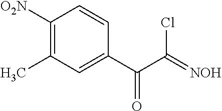

3-Methyl-4-nitroacetophenone (4.77 g) was dissolved in 4N hydrochloric acid-dioxane solution (26 mL), and while ice-cooled, isopentyl nitrite was added dropwise, with further stirring at room temperature for 24 hours. The reaction mixture was poured into saturated brine, extracted with t-butylmethylether and dried over anhydrous sodium sulfate, and the solvent was distilled off to obtain crude N-hydroxy-2-(3-methyl-4-nitrophenyl)-2-oxoethanimidoyl chloride (9.64 g).

¹H-NMR (CDCl₃, δ ppm): 2.63 (3H, s), 7.88-7.98 (3H, m).

### SYNTHESIS EXAMPLE 91-2

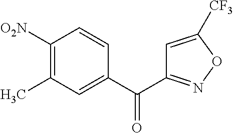

To an isopropanol solution (20 mL) of N-hydroxy-2-(3-methyl-4-nitrophenyl)-2-oxoethanimidoyl chloride (0.93 g) and 2-bromo-3,3,3-trifluoro-1-propane (1.31 g), sodium hydrogen carbonate (0.42 g) was added under ice cooling, and the mixture was stirred at room temperature for 15 hours. After the solvent was distilled off, water was added to the residue, which was then extracted with diethyl ether. The organic phase was washed with saturated brine, and dried over anhydrous sodium sulfate. The solvent was distilled off, and the crude product was purified with silica gel column chromatography (mixed solvent of n-hexane and ethyl acetate) to obtain (3-methyl-4-nitrophenyl)[5-(trifluoromethyl)isoxazol-3-yl]methanone (0.53 g).

¹H-NMR (CDCl₃, δ ppm): 2.68 (3H, s), 7.29 (1H, s), 8.06 (1H, d), 8.29-8.32 (2H, m).

### SYNTHESIS EXAMPLE 91-3

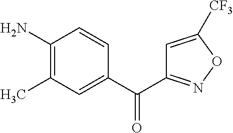

As similar to SYNTHESIS EXAMPLE 1-4, from (3-methyl-4-nitrophenyl)[5-(trifluoromethyl)isoxazol-3-yl]methanone (0.53 g), tin (II) chloride dihydrate (2.00 g), concentrated hydrochloric acid (2 mL) and ethanol (4 mL), (4-amino-3-methylphenyl)[5-(trifluoromethyl)isoxazol-3-yl]methanone (0.19 g) was obtained.

¹H-NMR (CDCl₃, δ ppm): 2.23 (3H, s), 4.31 (2H, bs), 6.70 (1H, d), 7.16 (1H, s), 8.07-8.11 (2H, m).

### SYNTHESIS EXAMPLE 92-1

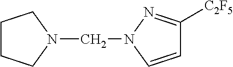

An ethanol solution (35 mL) of 3-(pentafluoroethyl)-1H-pyrazole (11.16 g), pyrrolidine (4.35 g) and 37% formaldehyde (5.45 g) was heated to reflux for 4 hours. After cooled at room temperature, the solvent was distilled off, and diethyl ether was added to the residue. The mixture was washed sequentially with saturated brine and saturated sodium chloride aqueous solution, and was dried over anhydrous sodium sulfate. The solvent was distilled off to obtain 3-(pentafluoroethyl)-1-(pyrrolidin-1-yl methyl)-1H-pyrazole (13.22 g).

¹H-NMR (CDCl₃, δ ppm): 1.70-1.80 (4H, m), 2.65-2.74 (4H, m), 5.11 (2H, s), 6.56 (1H, d), 7.53-7.57 (1H, m).

### SYNTHESIS EXAMPLE 92-2

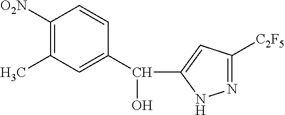

To a THF solution (40 mL) of 3-(pentafluoroethyl)-1-(pyrrolidin-1-ylmethyl)-1H-pyrazole (2.69 g), 15% n-butyllithiumhexane solution (6.8 mL) was added dropwise at −60° C. or lower, and the mixture was stirred at −70° C. or lower for 2 hours. Subsequently, a THF solution (20 mL) of 3-methyl-4-nitrobenzaldehyde (1.82 g) was added at −70° C. over 30 minutes, and was stirred at −70° C. for additional 30 minutes. After removing the dry ice bath, the mixture was gradually returned to room temperature with stirring for 4 hours. 2N hydrochloric acid was added to the mixture, which was then extracted with ethyl acetate. The organic phase was washed with saturated brine and dried over anhydrous sodium sulfate. The solvent was distilled off, and the crude product was subjected to silica gel column chromatography (mixed solvent of n-hexane and ethyl acetate) to obtain (3-methyl-4-nitrophenyl)[3-(pentafluoroethyl)-1H-pyrazol-5-yl]methanol (1.30 g).

¹H-NMR (CDCl₃, δ ppm): 1.42 (1H, s), 2.61 (3H, s), 6.02 (1H, s), 6.30 (1H, s), 7.31-7.43 (2H, m), 8.00 (1H, d).

### SYNTHESIS EXAMPLE 92-3

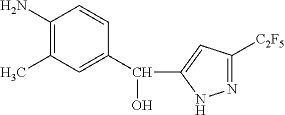

As similar to SYNTHESIS EXAMPLE 17-3, to (3-methyl-4-nitrophenyl)[3-(pentafluoroethyl)-1H-pyrazol-5-yl]methanol (0.39 g), hydrogeneration was carried out in the presence of a catalyst of 10% (w/w) palladium-carbon (0.06 g) to obtain (4-amino-3-methylphenyl)[3-(pentafluoroethyl)-1H-pyrazol-5-yl]methanol (0.32 g).

¹H-NMR (CDCl₃, δ ppm): 1.48-1.84 (3H, m), 2.17 (3H, s), 5.80 (1H, s), 6.28 (1H, s), 6.68 (1H, d), 6.99-7.08 (2H, m).

### SYNTHESIS EXAMPLE 92-4

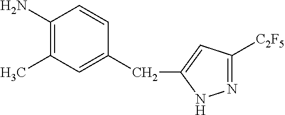

As similar to SYNTHESIS EXAMPLE 1-4, from (3-methyl-4-nitrophenyl)[3-(pentafluoroethyl)-1H-pyrazol-5-yl]methanol (0.35 g), tin (II) chloride dehydrate (1.13 g), concentrated hydrochloric acid (1.2 mL) and ethanol (2 mL), 2-methyl-4-{[3-(pentafluoroethyl)-1H-pyrazol-5-yl]methyl}aniline (0.28 g) was obtained.

¹H-NMR (CDCl₃, δ ppm): 2.15 (3H, s), 3.54-3.66 (3H, m), 3.98 (2H, s), 6.35 (1H, s), 6.66 (1H, d), 6.81-6.92 (2H, m).

### SYNTHESIS EXAMPLE 93-1

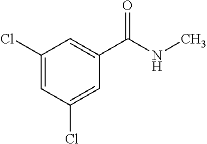

To a mixture of 40% methyl amine aqueous solution (2.33 g), 10% sodium hydroxide aqueous solution (28.80 g) and hexane (10 mL), 3,5-dichlorobenzoyl chloride (6.28 g) was added dropwise under ice-cooling over 5 minutes, and the mixture was stirred under ice-cooling for 30 minutes. The precipitated crude crystal was filtered, and after washed with water and a t-butylmethylether/petroleum ether mixed solvent and dried, 3,5-dichloro-N-methylbenzamide (4.70 g) was obtained.

¹H-NMR (CDCl₃, δ ppm): 3.00 (3H, s), 6.10 (1H, bs), 7.48 (1H, t), 7.63 (2H, d).

### SYNTHESIS EXAMPLE 93-2

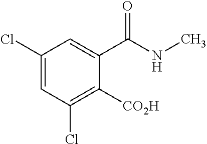

To a THF solution (50 mL) of 3,5-dichloro-N-methylbenzamide (4.08 g) and N,N,N′,N′-tetramethylethylene-diamine (5.11 g), 15% hexane solution (28.3 mL) of n-butyllithium was added dropwise at −70° C. Subsequently, the mixture was stirred at −70° C. for 1 hour. After dry ice (9 g) was added to the reaction mixture, the dry ice bath was removed, and the mixture was gradually returned to a room temperature with stirring for 1 hour and a half. The reaction mixture was acidified with 1N hydrochloric acid and extracted with ethyl acetate. The resulting organic phase was washed with saturated brine and dried with sodium sulfuric acid anhydride, and the solvent was distilled off to obtain 2,4-dichloro-6-(methylcarbamoyl)benzoic acid (3.82 g).

¹H-NMR (DMSO-d₆, δ ppm): 2.71 (3H, s), 7.68 (1H, s), 7.84 (1H, s), 8.61 (1H, s).

### SYNTHESIS EXAMPLE 93-3

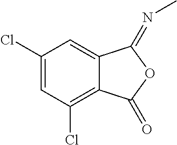

As similar to SYNTHESIS EXAMPLE 1-2, from 2,4-dichloro-6-(methylcarbamoyl)benzoic acid (1.99 g), sodium hydrogen carbonate (2.35 g) and chloromethyl carbonate (1.89 g), 5,7-dichloro-3-(methylimino)-2-benzofuran-1(3H)-one (1.54 g) was obtained.

¹H-NMR (CDCl₃, δ ppm): 3.42 (3H, s), 7.64 (1H, d), 7.81 (1H, d).

### SYNTHESIS EXAMPLE 94-1

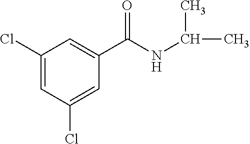

As similar to SYNTHESIS EXAMPLE 93-1, from isopropylamine (2.66 g), 10% sodium hydroxide aqueous solution (10 mL), hexane (20 mL) and 3,5-dichlorobenzoyl chloride (6.28 g), 3,5-dichloro-N-(isopropyl)benzamide (6.70 g) was obtained.

¹H-NMR (CDCl₃, δ ppm): 1.28 (6H, d), 4.19-4.33 (1H, m), 5.75-5.90 (1H, m), 7.47 (1H, t), 7.61 (2H, d).

### SYNTHESIS EXAMPLE 94-2

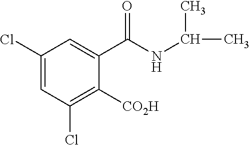

As similar to SYNTHESIS EXAMPLE 93-2, from 15% hexane solution (15.2 mL) of 3,5-dichloro-N-(propan-2-yl)benzamide (2.32 g), N,N,N′,N′-tetra methylethylenediamine (2.56 g), n-butyl lithium and dry ice (9 g), 2,4-dichloro-6-[2-(1-methylethyl)carbamoyl]benzoic acid (2.35 g) was obtained.

¹H-NMR (DMSO-d₆, δ ppm): 1.07 (6H, d), 3.82-3.98 (1H, m), 7.50 (1H, bs), 7.77 (1H, bs), 8.41 (1H, d), 13.18-13.51 (1H, m).

### SYNTHESIS EXAMPLE 94-3

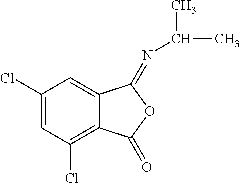

As similar to SYNTHESIS EXAMPLE 1-2, from 2,4-dichloro-6-[2-(1-methylethyl)carbamoyl]benzoic acid (2.35 g), sodium hydrogen carbonate (0.86 g) and methyl chlorocarbonate (0.97 g), 5,7-dichloro-3-(isopropylimino)-2-benzofuran-1(3H)-one (0.90 g) was obtained.

¹H-NMR (CDCl₃, δ ppm): 1.27 (6H, d), 4.21-4.34 (1H, m), 7.63 (1H, d), 7.86 (1H, d).

### SYNTHESIS EXAMPLE 95-1

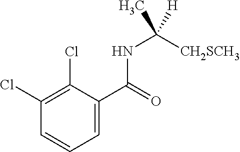

To a THF solution (60 mL) of 2,3-dichloro benzoic acid (2.87 g), 4-dimethylaminopyridine (0.55 g) and 1-ethyl-3-(3-dimethylaminopropyl)carbodiimide hydrochloride (4.31 g), (2S)-1-(methylthio) propan-2-amine (1.58 g) was added at room temperature. After stirring at room temperature for 6 hours, the mixture was washed with 2N hydrochloric acid and saturated aqueous solution of sodium hydrogen carbonate and dried over anhydrous sodium sulfate. The solvent was distilled off to obtain 2,3-dichloro-N-[(1S)-1-methyl-2-(methylthio)ethyl]-benzamide (3.82 g).

¹H-NMR (CDCl₃, δ ppm): 1.36 (3H, d), 2.18 (3H, s), 2.68-2.82 (2H, m), 4.35-4.48 (1H, m), 6.05-6.19 (1H, m), 7.23-7.56 (3H, m).

### SYNTHESIS EXAMPLE 95-2

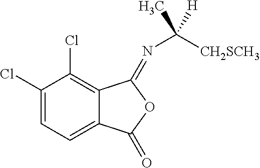

To a diethyl ether solution (100 mL) of 2,3-dichloro-N-[(1S)-1-methyl-2-(methylthio)ethyl]benzamide (2.78 g) and N,N,N′,N′-tetramethylethylenediamine (2.56 g), 15% hexane solution of n-butyl lithium (13.6 mL) was added dropwise at −70° C. Then, after stirring at −70° C. for 1 hour, an excessive amount of carbon dioxide was blown into the reaction mixture. The dry ice bath was removed, and the mixture was gradually returned to room temperature, with stirring for addition 1 hour, acidified with 2N hydrochloric acid, and then extracted with ethyl acetate. The organic phase was washed with saturated brine, and dried over anhydrous sodium sulfate, and the solvent was distilled off to obtain crude 3,4-dichloro-2-{[(1S)-1-methyl-2-(methylthio)ethyl]carbamoyl}benzoic acid. This crude product was dissolved in ethyl acetate (20 mL), and saturated aqueous solution (10 mL) of sodium hydrogen carbonate and methyl chlorocarbonate (2.36 g) were added, and the mixture was stirred at 50° C. for 30 minutes. The organic phase was separated, washed with saturated aqueous solution of sodium hydrogen carbonate, and was dried over anhydrous sodium sulfate. The solvent was distilled off, and the resulting crude product was subjected to a silica gel column chromatography (n-hexane, ethyl acetate mixed solvent) to obtain 4,5-dichloro-3-{[(1S)-1-methyl-2-(methylthio)ethyl]imino}-2-benzofuran-1(3H)-one (0.37 g).

¹H-NMR (CDCl₃, δ ppm): 1.36 (3H, d), 2.18 (3H, s), 2.71-2.82 (2H, m), 4.30-4.44 (1H, m), 7.79 (2H, bs).

### SYNTHESIS EXAMPLE 96-1

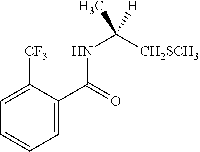

To a THF solution (40 mL) of (2S)-1-(methylthio)propan-2-amine (2.31 g) and triethylamine (3.35 mL), 2-(trifluoromethyl)benzoyl chloride (4.17 g) was added at 5° C., and the mixture was stirred at the same temperature for 1 hour. Then, 2N hydrochloric acid (20 mL) and ethyl acetate (60 mL) were added to the reaction mixture, and the organic phase was separated, washed with 2N hydrochloric acid and saturated aqueous solution of sodium hydrogen carbonate, and dried over anhydrous sodium sulfate. The solvent was distilled off to obtain N-[(1S)-1-methyl-2-(methylthio)ethyl]-2-(trifluoromethyl)benzamide (4.18 g).

¹H-NMR (CDCl₃, δ ppm): 1.34 (3H, d), 2.18 (3H, s), 2.68 (1H, dd), 2.77 (1H, dd), 4.33-4.48 (1H, m), 5.83-5.97 (1H, m), 7.48-7.75 (4H, m).

### SYNTHESIS EXAMPLE 96-2

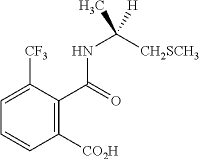

As similar to SYNTHESIS EXAMPLE 93-2, from N-[(1S)-1-methyl-2-(methylthio)ethyl]-2-(trifluoromethyl)benzamide (1.39 g), N,N,N′,N′-tetramethylethylenediamine (1.28 g), 15% n-butyllithium hexane solution (6.7 mL), dry ice (2.2 g), 3-(trifluoromethyl)-2-{[(1S)-1-methyl-2-(methylthio)ethyl]carbamoyl}benzoic acid (1.40 g) was obtained.

¹H-NMR (CDCl₃, δ ppm): 1.34 (3H, d), 2.17 (3H, s), 2.65-2.80 (2H, m), 4.30-4.49 (1H, m), 5.86-6.59 (2H, m), 7.48-7.65 (3H, m).

### SYNTHESIS EXAMPLE 96-3

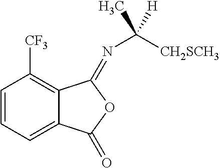

As similar to SYNTHESIS EXAMPLE 1-2, from 3-(trifluoromethyl)-2-{[(1S)-1-methyl-2-(methylthio)ethyl]carbamoyl}benzoic acid (1.50 g), sodium hydrogen carbonate (1.96 g) and methyl chlorocarbonate (2.21 g), 3-{[(1S)-1-methyl-2-(methylthio)ethyl]imino}-4-(trifluoromethyl)-2-benzofuran-1(3H)-one (0.55 g) was obtained.

¹H-NMR (CDCl₃, δ ppm): 1.35 (3H, d), 2.14 (3H, s), 2.73 (2H, d), 4.30-4.43 (1H, m), 7.82 (1H, dd), 8.09 (1H, d), 8.15 (1H, d).

### SYNTHESIS EXAMPLE 97-1

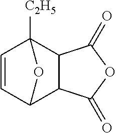

2-Ethyl furan (10.68 g) and maleic anhydride (9.81 g) were dissolved in dehydrated diethyl ether (35 mL), and were left at room temperature for 16 hours. The reaction mixture was cooled to −5° C., and the precipitated crystal was collected by filtration, and then washed with a small amount of diethyl ether to obtain 4-ethyl-3a,4,7,7a-tetrahydro-4,7-epoxy-2-benzofuran-1,3-dione (18.0 g).

¹H-NMR (CDCl₃, δ ppm): 1.16 (3H, t), 2.07-2.17 (2H, m), 3.08 (1H, d), 3.29 (1H, d), 5.37 (1H, bs), 6.34 (1H, d), 6.57 (1H, d).

### SYNTHESIS EXAMPLE 97-2

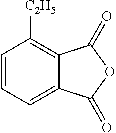

A mixed solvent of concentrated sulfuric acid (68 mL) and sulfolane (27 mL) was cooled to −55° C., and a powder of 4-ethyl-3a,4,7,7a-tetrahydro-4,7-epoxy-2-benzofuran-1,3-dione (17.5 g) was added in portions so that the temperature did not exceed −45° C. Then, the mixture was stirred at a temperature between −55° C. and −45° C. for 3 hours. The dry ice bath was removed, and the reaction mixture was gradually returned to room temperature with stirring for additional 3 hours. After the reaction mixture was poured into iced water, the precipitated crystal was filtered and washed with cold water and dried to obtain a mixture (12.6 g) of 3-ethylphthalic anhydride and 3-ethylphthalic acid. This mixture was heated to reflux in acetic anhydride (6.7 mL) for 2 hours. After an insoluble matter was filtered off in the hot state, the filtrate was left overnight at room temperature, and the precipitated crude crystal was washed with a t-butylmethylether/petroleum ether mixed solvent to obtain 3-ethylphthalic anhydride (6.1 g).

¹H-NMR (CDCl₃, δ ppm): 1.28 (3H, t), 2.73 (2H, q), 7.35-7.53 (2H, m), 7.94 (1H, d).

### SYNTHESIS EXAMPLE 98-2

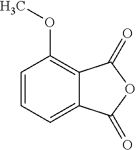

After 2,3-dimethyl anisole (10 g) was dissolved in a mixed solvent of water (250 mL) and t-butanol (112 mL), potassium permanganate (81.2 g) was added, and the mixture was heated to reflux for 6 hours. After cooling to room temperature, an insoluble matter was filtered with Celite, followed by washing with water. After the filtrate was acidified with concentrated hydrochloric acid and concentrated under reduced pressure, a mixture (4.9 g) of 3-methoxyphthalic anhydride and 3-methoxyphthalic acid was obtained. This mixture was heated to reflux in acetic anhydride (8.5 mL) for 2 hours. After an insoluble matter was filtered off in the hot state, the filtrate was left overnight at room temperature, and the precipitated crude crystal was washed with a t-butylmethylether/petroleum ether mixed solvent to obtain 3-methoxyphthalic anhydride (3.21 g).

¹H-NMR (CDCl₃, δ ppm): 4.08 (3H, s), 7.33 (1H, d), 7.58 (1H, d), 7.84 (1H, dd).

### SYNTHESIS EXAMPLE 99-1

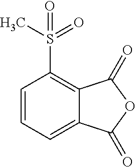

As similar to SYNTHESIS EXAMPLE 98-1, from 2,3-dimethylthioanisole (5.0 g), water (140 mL), t-butanol (60 mL), potassium permanganate (33.2 g) and acetic anhydride (100 mL), 3-methanesulfonyl phthalic anhydride (3.40 g) was obtained.

¹H-NMR (DMSO-d₆, δ ppm): 3.30 (3H, s), 7.75 (1H, dd), 8.17 (2H, d).

### SYNTHESIS EXAMPLE 100-1

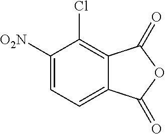

As similar to SYNTHESIS EXAMPLE 98-1, from 2-chloro-3,4-dimethylnitrobenzene (4.5 g), water (120 mL), t-butanol (60 mL), potassium permanganate (25.7 g) and acid anhydride (100 mL), 3-chloro-4-nitrophthalic anhydride (4.30 g) was obtained.

¹H-NMR (DMSO-d₆, δ ppm): 8.13 (1H, d), 8.38 (1H, d).

### SYNTHESIS EXAMPLE 101-1

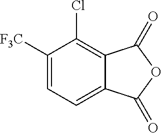

As similar to SYNTHESIS EXAMPLE 98-1, from 2-chloro-3,4-dimethylbenzotrifluoride (5.0 g), water (140 mL), t-butanol (60 mL), potassium permanganate (25.4 g) and acetic anhydride (50 mL), 3-chloro-4-(trifluoromethyl)phthalic anhydride (6.13 g) was obtained.

¹H-NMR (DMSO-d₆, δ ppm): 8.11 (1H, d), 8.32 (1H, d).

### SYNTHESIS EXAMPLE 102-1

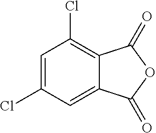

As similar to SYNTHESIS EXAMPLE 98-1, from 1,5-dichloro-2,3-dimethylbenzene (3.50 g), water (98 mL), t-butanol (42 mL), potassium permanganate (21.2 g) and acetic anhydride (100 mL), 3,5-dichlorophthalic anhydride (2.80 g) was obtained.

¹H-NMR (DMSO-d₆, δ ppm): 8.00 (2H, s).

### SYNTHESIS EXAMPLE 103-1

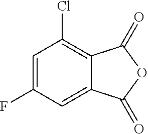

As similar to SYNTHESIS EXAMPLE 98-1, from 1-chloro-5-fluoro-2,3-dimethylbenzene (10.0 g), water (500 mL), t-butanol (214 mL), potassium permanganate (66.8 g) and acetic anhydride (100 mL), 3-chloro-5-fluorophthalic anhydride (3.35 g) was obtained.

¹H-NMR (DMSO-d₆, δ ppm): 7.72-7.78 (2H, m).

### SYNTHESIS EXAMPLE 104-1

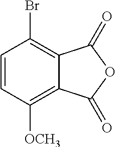

As similar to SYNTHESIS EXAMPLE 98-1, from 4-bromo-2,3-dimethylanisole (7.0 g), water (200 mL), t-butanol (80 mL), potassium permanganate (32.9 g) and acetic anhydride (120 mL), 3-bromo-6-methoxyphthalic anhydride (3.1 g) was obtained.

¹H-NMR (DMSO-d₆, δ ppm): 4.02 (3H, s), 7.39 (1H, d), 7.98 (1H, d).

### SYNTHESIS EXAMPLE 105-1

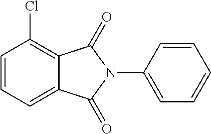

An acetic acid (200 mL) solution of 3-chlorophthalic anhydride (36.5 g) and aniline (18.6 g) was heated to reflux for 3 hours. After cooling to room temperature, the reaction mixture was poured into iced water, and the precipitated crystal was collected by filtration, washed with water and 5% isopropanol aqueous solution, and then dried. The resulting crude crystal was washed with a t-butylmethylether/petroleum ether mixed solvent to obtain 3-chloro-N-phenylphthalimide (50.1 g).

¹H-NMR (CDCl₃, δ ppm): 7.38-7.55 (5H, m), 7.69-7.74 (2H, m), 7.85-7.91 (1H, m).

### SYNTHESIS EXAMPLE 105-2

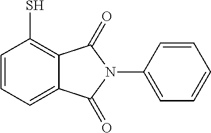

3-Chloro-N-phenylphthalimide (64.4 g) was dissolved in DMF (770 mL) under argon atmosphere at room temperature, and sodium hydrosulfide monohydrate (60.07 g) and water (445 mL) were added thereto. Then, the mixture was heated and stirred at 85° C. for 6 hours. After cooled to room temperature, the reaction mixture was diluted with water, and saturated aqueous solution of sodium hydrogen carbonate was added to adjust the pH to 9 or above. An insoluble matter was filtered off, and the filtrate was washed with ethyl acetate and acidified with citric acid. The precipitated crystal was collected by filtration, and washed with water and petroleum ether to obtain 3-mercapto-N-phenylphthalimide (18.3 g).

¹H-NMR (CDCl₃, δ ppm): 6.22 (1H, s), 7.36-7.71 (8H, m).

### SYNTHESIS EXAMPLE 105-3

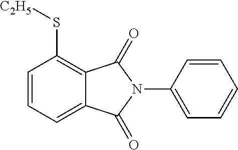

To an acetonitrile solution (80 mL) of 3-mercapto-N-phenylphthalimide (10.21 g), ethyl iodide (9.36 g) and potassium carbonate (8.29 g) were added, and the mixture was heated to reflux for 3 hours. The reaction mixture was poured into iced water, and the precipitated crystal was filtered, washed with water and dried to obtain 3-ethylthio-N-phenylphthalimide (10.13 g).

¹H-NMR (CDCl₃, δ ppm): 1.46 (3H, t), 3.11 (2H, q), 7.35-7.69 (8H, m).

### SYNTHESIS EXAMPLE 105-4

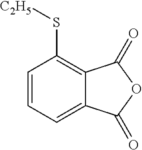

3-Ethylthio-N-phenyl phthalimide (5.0 g) and a 30% sodium hydroxide aqueous solution (18.8 g) were charged into a 100 mL autoclave, and then the mixture was overheated and stirred at 145° C. for 6 hours. After cooling to room temperature, the reaction mixture was diluted with water, washed with t-butylmethylether, acidified with concentrated hydrochloric acid, and then extracted with ethyl acetate. After the organic phase was dried over anhydrous sodium sulfate, the solvent was distilled off, and the obtained crude 3-ethyl thiophthalic acid (3.0 g) was heated to reflux in acetic anhydride (5 mL) for 2 hours. After the solvent was distilled off, the crude product was washed with a mixed solvent of t-butylmethylether/petroleum ether to obtain 3-ethylthiophthalic anhydride (2.0 g).

¹H-NMR (CDCl₃, δ ppm): 1.47 (3H, t), 3.13 (2H, q), 7.61 (1H, d), 7.67-7.79 (2H, m).

### SYNTHESIS EXAMPLE 106-1

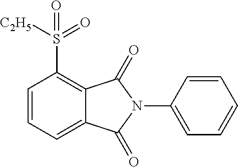

To an acetic acid solution (45 mL) of 3-ethyl thio-N-phenylphthalimide (5.67 g), 30% hydrogen peroxide solution (6.1 mL) was added, and the mixture was heated to reflux at 70° C. for 8 hours. The reaction mixture was poured into iced water, and the precipitated crystal was filtered, washed sequentially with water, saturated aqueous solution of sodium hydrogen carbonate and water, and dried to obtain 3-ethylsulfonyl-N-phenylphthalimide (4.79 g).

¹H-NMR (CDCl₃, δ ppm): 1.45 (3H, t), 3.73 (2H, q), 7.39-7.57 (5H, m), 8.01 (1H, dd), 8.24 (1H, d), 8.46 (1H, d).

### SYNTHESIS EXAMPLE 106-2

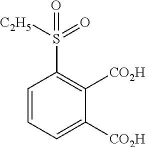

3-Ethylsulfonyl-N-phenylphthalimide (7.88 g) and 10% sodium hydroxide aqueous solution (30 g) were heated to reflux for 2 hours, and then cooled in the ice bath. The mixture was acidified with concentrated hydrochloric acid and further stirred at 60° C. for 1 hour and a half.

After the reaction mixture was cooled to room temperature, it was extracted with ethyl acetate. The organic phase was washed with saturated brine, and dried over anhydrous sodium sulfate, and the solvent was distilled off to obtain 3-ethylsulfonylphthalic acid (4.30 g).

¹H-NMR (DMSO-d₆, δ ppm): 1.14 (3H, t), 3.45 (2H, q), 7.80 (1H, dd), 8.13 (1H, d), 8.21 (1H, d), 13.12-14.15 (1H, m).

### SYNTHESIS EXAMPLE 106-3

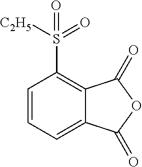

3-Ethylsulfonyl phthalic acid (6.00 g) was heated to reflux in acetic anhydride (8.8 mL) for 3 hours. An insoluble matter was filtered off in the hot state. The filtrate was cooled to room temperature, and the precipitated crystal was collected by filtration and washed with t-butylmethylether to obtain 3-ethylsulfonylphthalic anhydride (3.31 g).

¹H-NMR (CDCl₃, δ ppm): 1.36 (3H, t), 3.65 (2H, q), 8.14 (1H, dd), 8.32 (1H, d), 8.56 (1H, d).

Examples of Compounds of Formula (I)

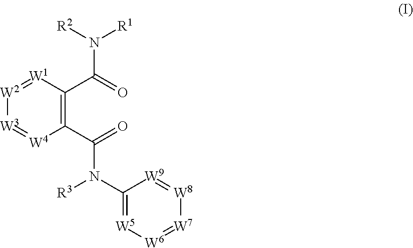

which are obtained by (similar) processes as described in the SYNTHESIS EXAMPLES are given in the following tables.
As the substituent listed in the column of the group R¹, the groups R¹ a and the like represent the following moieties:

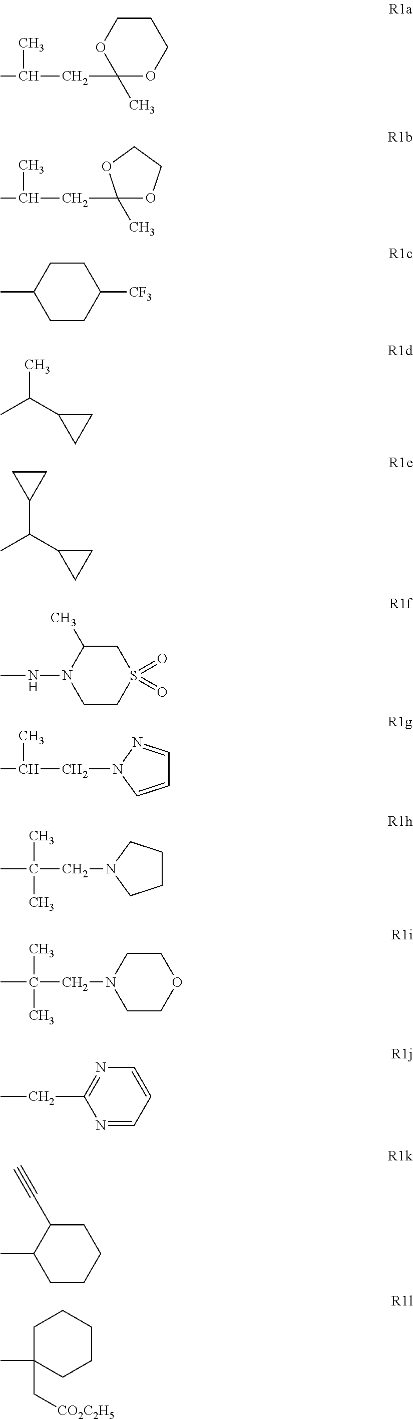

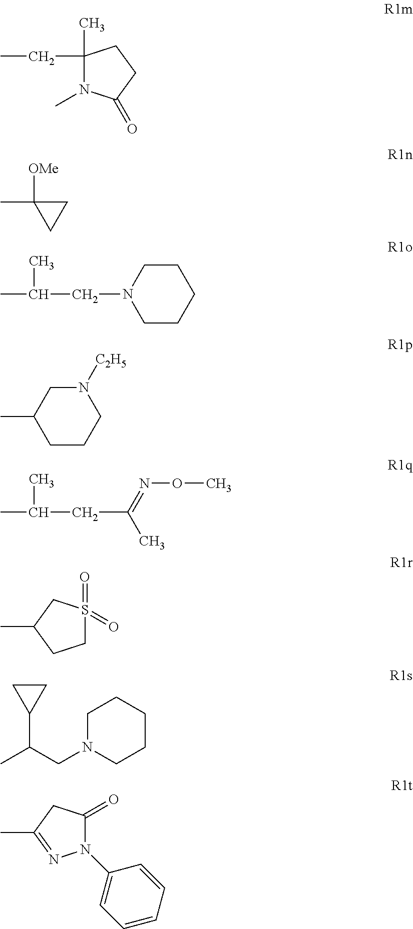

The invention is illustrated be the examples given in the tables. If not mentioned otherwise, the tables show compounds having the formula (I).

In the table, mp is the abbreviation for melting point; logP(acid) is a measurement value determined as described in the EEC Direction 79/831 annexed documents V. A8 by reverse phase (C18)-chromatography at pH2.3 using a mobile phase of 0.1% (w/w) phosphoric acid aqueous solution and acetonitrile with a linear gradient of 10-95% acetonitrile; and logP(neutral) value is a measurement value determined by determined as described in the EEC Direction 79/831 annexed documents V. A8 by using a mobile phase of 0.0025M potassium dihydrogen phosphate aqueous solution and acetonitrile with a linear gradient of 10-95% acetonitrile. As for the compounds in which NMR values are shown as “physical property” column, NMR values are given shown in Table 17.

TABLE 1

| No. | R¹ | R² | R³ | W¹ | W² | W³ | W⁴ | W⁵ |
|---|---|---|---|---|---|---|---|---|
| 1-1 | C₂H₅ | C₂H₅ | H | C—Cl | CH | CH | CH | C—CH₃ |
| 1-2 | C₂H₅ | C₂H₅ | H | C—Cl | CH | CH | CH | C—CH₃ |
| 1-3 | C₂H₅ | C₂H₅ | H | C—Cl | CH | CH | CH | C—CH₃ |
| 1-4 | C₂H₅ | C₂H₅ | H | C—Cl | CH | CH | CH | C—CH₃ |
| 1-5 | C₂H₅ | C₂H₅ | H | C—Cl | CH | CH | CH | C—CH₃ |
| 1-6 | C₂H₅ | C₂H₅ | H | C—Cl | CH | CH | CH | C—CH₃ |
| 1-7 | CH(CH₃)₂ | CH₃ | H | C—Cl | CH | CH | CH | C—CH₃ |
| 1-8 | C₃H₇-n | C₂H₅ | H | C—Cl | CH | CH | CH | C—CH₃ |
| 1-9 | CH(CH₃)₂ | CH₃ | H | C—Cl | CH | CH | CH | C—CH₃ |
| 1-10 | CH(CH₃)CH₂SCH₃ (S)-isomer | CH₃ | H | C—Cl | CH | CH | CH | C—CH₃ |
| 1-11 | CH(CH₃)CH₂SOCH₃ (S)-isomer | CH₃ | H | C—Cl | CH | CH | CH | C—CH₃ |
| 1-12 | CH(CH₃)₂ | C₂H₅ | H | C—Cl | CH | CH | CH | C—CH₃ |
| 1-13 | C₄H₉-n | C₂H₅ | H | C—Cl | CH | CH | CH | C—CH₃ |
| 1-14 | CH(CH₃)CH₂SO₂CH₃ (S)-isomer | CH₃ | H | C—Cl | CH | CH | CH | C—CH₃ |
| 1-15 | CH(CH₃)CH₂SCH₃ (S)-isomer | C₂H₅ | H | C—Cl | CH | CH | CH | C—CH₃ |
| 1-16 | CH(CH₃)CH₂SCH₃ (S)-isomer | C₂H₅ | H | C—SCH₃ | CH | CH | CH | C—CH₃ |
| 1-17 | CH(CH₃)CH₂SCH₃ (S)-isomer | CH₃ | H | C—Cl | CH | CH | CH | C—CH₃ |
| 1-18 | CH(CH₃)CH₂SOCH₃ (S)-isomer | CH₃ | H | C—Cl | CH | CH | CH | C—CH₃ |
| 1-19 | CH(CH₃)CH₂SO₂CH₃ (S)-isomer | CH₃ | H | C—Cl | CH | CH | CH | C—CH₃ |
| 1-20 | CH(CH₃)CH₂SCH₃ (S)-isomer | CH₃ | H | C—I | CH | CH | CH | C—CH₃ |
| 1-21 | OCH₃ | CH₃ | H | C—I | CH | CH | CH | C—CH₃ |
| 1-22 | CH₂CH═CH₂ | CH₂CH═CH₂ | H | C—I | CH | CH | CH | C—CH₃ |

| No. | W⁶ | W⁷ | W⁸ | W⁹ | A | r | Q | R⁴ |
|---|---|---|---|---|---|---|---|---|
| 1-1 | CH | C-(A)r-Q | CH | CH | CH₂ | 1 | Q8 | CF₃ |
| 1-2 | CH | C-(A)r-Q | CH | CH | CH₂ | 1 | Q8 | CF₃ |
| 1-3 | CH | C-(A)r-Q | CH | CH | CH₂ | 1 | Q34 | CF₃ |
| 1-4 | CH | C-(A)r-Q | CH | CH | CH₂ | 1 | Q34 | C₂F₅ |
| 1-5 | CH | C-(A)r-Q | CH | CH | CH₂ | 1 | Q34 | CF₃ |
| 1-6 | CH | C-(A)r-Q | CH | CH | CH₂ | 1 | Q34 | C₂F₅ |
| 1-7 | CH | C-(A)r-Q | CH | CH | CH₂ | 1 | Q8 | CF₃ |
| 1-8 | CH | C-(A)r-Q | CH | CH | CH₂ | 1 | Q34 | CF₃ |
| 1-9 | CH | C-(A)r-Q | CH | CH | CH₂ | 1 | Q34 | CF₃ |
| 1-10 | CH | C-(A)r-Q | CH | CH | CH₂ | 1 | Q8 | CF₃ |
| 1-11 | CH | C-(A)r-Q | CH | CH | CH₂ | 1 | Q8 | CF₃ |
| 1-12 | CH | C-(A)r-Q | CH | CH | CH₂ | 1 | Q8 | CF₃ |
| 1-13 | CH | C-(A)r-Q | CH | CH | CH₂ | 1 | Q8 | C₂F₅ |
| 1-14 | CH | C-(A)r-Q | CH | CH | CH₂ | 1 | Q8 | CF₃ |
| 1-15 | CH | C-(A)r-Q | CH | CH | CH₂ | 1 | Q8 | CF₃ |
| 1-16 | CH | C-(A)r-Q | CH | CH | CH₂ | 1 | Q8 | CF₃ |
| 1-17 | CH | C-(A)r-Q | CH | CH | CH₂ | 1 | Q34 | CF₃ |
| 1-18 | CH | C-(A)r-Q | CH | CH | CH₂ | 1 | Q34 | CF₃ |
| 1-19 | CH | C-(A)r-Q | CH | CH | CH₂ | 1 | Q34 | CF₃ |
| 1-20 | CH | C-(A)r-Q | CH | CH | CH₂ | 1 | Q48 | 3,5- |
| | | | | | | | | bis(trifluoromethyl)phenyl |
| 1-21 | CH | C-(A)r-Q | CH | CH | CH₂ | 1 | Q48 | 3,5- |
| | | | | | | | | bis(trifluoromethyl)phenyl |
| 1-22 | CH | C-(A)r-Q | CH | CH | CH₂ | 1 | Q48 | 3,5- |
| | | | | | | | | bis(trifluoromethyl)phenyl |

| No. | R¹ | R² | R³ | W¹ | W² | W³ | W⁴ | W⁵ | W⁶ | W⁷ |
|---|---|---|---|---|---|---|---|---|---|---|
| 1-23 | —CH₂CH₂CH₂CH₂— | | H | C—I | CH | CH | CH | C—CH₃ | CH | C-(A)r-Q |
| 1-24 | —CH₂CH₂CH₂CH₂CH₂— | | H | C—I | CH | CH | CH | C—CH₃ | CH | C-(A)r-Q |
| 1-25 | —CH₂CH₂SCH₂— | | H | C—I | CH | CH | CH | C—CH₃ | CH | C-(A)r-Q |
| 1-26 | —CH₂CH₂OCH₂CH₂— | | H | C—Cl | CH | CH | CH | C—CH₃ | CH | C-(A)r-Q |
| 1-27 | —CH₂CH₂SCH₂CH₂— | | H | C—Cl | CH | CH | CH | C—CH₃ | CH | C-(A)r-Q |
| 1-28 | —CH₂CH₂SO₂CH₂CH₂— | | H | C—Cl | CH | CH | CH | C—CH₃ | CH | C-(A)r-Q |
| 1-29 | —CH₂CH₂OCH₂CH₂— | | H | CH | CH | CH | C—Cl | C—CH₃ | CH | C-(A)r-Q |
| 1-30 | —CH₂CH₂SCH₂CH₂— | | H | CH | CH | CH | C—Cl | C—CH₃ | CH | C-(A)r-Q |
| 1-31 | —CH₂CH₂SO₂CH₂CH₂— | | H | CH | CH | CH | C—Cl | C—CH₃ | CH | C-(A)r-Q |
| 1-32 | C₂H₅ | C₂H₅ | H | C—CHO | CH | CH | CH | C—CH₃ | CH | C-(A)r-Q |
| 1-33 | C₂H₅ | C₂H₅ | H | C—CO₂C₂H₅ | CH | CH | CH | C—CH₃ | CH | C-(A)r-Q |
| 1-34 | C₂H₅ | C₂H₅ | H | C—CONH₂ | CH | CH | CH | C—CH₃ | CH | C-(A)r-Q |
| 1-35 | C₂H₅ | C₂H₅ | H | C—COCH₃ | CH | CH | CH | C—CH₃ | CH | C-(A)r-Q |
| 1-36 | C₂H₅ | C₂H₅ | H | C—CONHC₂H₅ | CH | CH | CH | C—CH₃ | CH | C-(A)r-Q |
| 1-37 | C₂H₅ | C₂H₅ | H | C—CON(CH₃)₂ | CH | CH | CH | C—CH₃ | CH | C-(A)r-Q |
| 1-38 | C₂H₅ | C₂H₅ | H | C—CON(C₂H₅)₂ | CH | CH | CH | C—CH₃ | CH | C-(A)r-Q |

| | | | | | | | | | | Physical |
|---|---|---|---|---|---|---|---|---|---|---|
| No. | W⁸ | W⁹ | A | r | Q | R⁴ | R⁵ | R⁶ | R⁷ | property |
| 1-23 | CH | CH | CH₂ | 1 | Q8 | C₂F₅ | H | H | — | |
| 1-24 | CH | CH | CH₂ | 1 | Q8 | C₂F₅ | H | H | — | |
| 1-25 | CH | CH | CH₂ | 1 | Q8 | C₂F₅ | H | H | — | |
| 1-26 | CH | CH | CH₂ | 1 | Q8 | CF₃ | H | CF₃ | — | |
| 1-27 | CH | CH | CH₂ | 1 | Q8 | CF₃ | H | CF₃ | — | |
| 1-28 | CH | CH | CH₂ | 1 | Q8 | CF₃ | H | CF₃ | — | |
| 1-29 | CH | CH | CH₂ | 1 | Q8 | CF₃ | H | CF₃ | — | NMR |
| 1-30 | CH | CH | CH₂ | 1 | Q8 | CF₃ | H | CF₃ | — | |
| 1-31 | CH | CH | CH₂ | 1 | Q8 | CF₃ | H | CF₃ | — | |
| 1-32 | CH | CH | CH₂ | 1 | Q8 | CF₃ | H | H | — | |
| 1-33 | CH | CH | CH₂ | 1 | Q8 | CF₃ | H | H | — | |
| 1-34 | CH | CH | CH₂ | 1 | Q8 | CF₃ | H | H | — | |
| 1-35 | CH | CH | CH₂ | 1 | Q8 | CF₃ | H | H | — | |
| 1-36 | CH | CH | CH₂ | 1 | Q8 | CF₃ | H | H | — | |
| 1-37 | CH | CH | CH₂ | 1 | Q8 | CF₃ | H | H | — | |
| 1-38 | CH | CH | CH₂ | 1 | Q8 | CF₃ | H | H | — | |

TABLE 2

| No. | R¹ | R² | R³ | W¹ | W² | W³ | W⁴ | W⁵ | W⁶ |
|---|---|---|---|---|---|---|---|---|---|
| 2-1 | CH(CH₃)₂ | H | C₂H₅ | CH | CH | CH | CH | C—CH₃ | CH |
| 2-2 | CH(CH₃)₂ | H | C₂H₅ | CH | CH | CH | CH | C—CH₃ | CH |
| 2-3 | CH(CH₃)₂ | H | C₂H₅ | C—Cl | CH | CH | CH | C—CH₃ | CH |
| 2-4 | CH(CH₃)₂ | H | C₂H₅ | C—Cl | CH | CH | CH | C—CH₃ | CH |
| 2-5 | CH(CH₃)₂ | H | C₂H₅ | C—SCH₃ | CH | CH | CH | C—CH₃ | CH |
| 2-6 | CH(CH₃)₂ | H | C₂H₅ | C—SO₂C₂H₅ | CH | CH | CH | C—CH₃ | CH |
| 2-7 | C(CH₃)₂CH₂SCH₃ | H | C₂H₅ | C—I | CH | CH | CH | C—CH₃ | CH |
| 2-8 | C(CH₃)₂CH₂SO₂CH₃ | H | C₂H₅ | C—I | CH | CH | CH | C—CH₃ | CH |
| 2-9 | C(CH₃)₂CH₂SCH₃ | H | C₂H₅ | C—I | CH | CH | CH | C—CH₃ | CH |
| 2-10 | C(CH₃)₂CH₂SO₂CH₃ | H | C₂H₅ | C—I | CH | CH | CH | C—CH₃ | CH |
| 2-11 | C(CH₃)₂CH₂SCH₃ | H | C₂H₅ | C—I | CH | CH | CH | C—CH₃ | CH |
| 2-12 | C(CH₃)₂CH₂SO₂CH₃ | H | C₂H₅ | C—I | CH | CH | CH | C—CH₃ | CH |
| 2-13 | CH(CH₃)CH₂SCH₃ (S)-isomer | H | CH₃ | CH | CH | CH | CH | C—CH₃ | CH |
| 2-14 | CH(CH₃)CH₂SCH₃ (S)-isomer | H | CH₃ | C—Cl | CH | CH | CH | C—CH₃ | CH |
| 2-15 | CH(CH₃)CH₂SCH₃ (S)-isomer | H | C₂H₅ | C—Cl | CH | CH | CH | C—CH₃ | CH |
| 2-16 | CH(CH₃)CH₂SCH₃ (S)-isomer | H | CH₃ | C—Cl | CH | CH | CH | C—CH₃ | CH |
| 2-17 | CH(CH₃)CH₂SCH₃ (S)-isomer | H | CH₃ | C—Cl | CH | CH | CH | C—CH₃ | CH |
| 2-18 | CH(CH₃)CH₂SCH₃ (S)-isomer | H | CH₃ | C—Cl | CH | CH | CH | C—CH₃ | CH |
| 2-19 | CH(CH₃)CH₂SCH₃ (S)-isomer | H | CH₃ | C—Cl | CH | CH | CH | C—CH₃ | CH |
| 2-20 | CH(CH₃)CH₂SCH₃ (S)-isomer | H | C₂H₅ | C—Cl | CH | CH | CH | C—CH₃ | CH |
| 2-21 | CH(CH₃)CH₂SCH₃ (S)-isomer | H | C₃H₇-n | C—Cl | CH | CH | CH | C—CH₃ | CH |
| 2-22 | CH(CH₃)CH₂SCH₃ (S)-isomer | H | CH₂C≡CH | C—Cl | CH | CH | CH | C—CH₃ | CH |
| 2-23 | CH(CH₃)CH₂SCH₃ (S)-isomer | H | CH₂CH₂OCH₃ | C—Cl | CH | CH | CH | C—CH₃ | CH |
| 2-24 | CH(CH₃)CH₂SCH₃ (S)-isomer | H | CH₂CH₂SCH₃ | C—Cl | CH | CH | CH | C—CH₃ | CH |

| | | | | | | | | | | | Physical |
|---|---|---|---|---|---|---|---|---|---|---|---|
| No. | W⁷ | W⁸ | W⁹ | A | r | Q | R⁴ | R⁵ | R⁶ | R⁷ | property |
| 2-1 | C—(A)r—Q | CH | CH | CH₂ | 1 | Q8 | C₂F₅ | H | H | — | |
| 2-2 | C—(A)r—Q | CH | CH | CH₂ | 1 | Q34 | CF₃ | H | — | — | |
| 2-3 | C—(A)r—Q | CH | CH | CH₂ | 1 | Q8 | C₂F₅ | H | H | — | |
| 2-4 | C—(A)r—Q | CH | CH | CH₂ | 1 | Q34 | CF₃ | H | — | — | |
| 2-5 | C—(A)r—Q | CH | CH | CH₂ | 1 | Q8 | C₂F₅ | H | H | — | |
| 2-6 | C—(A)r—Q | CH | CH | CH₂ | 1 | Q34 | C₂F₅ | H | — | — | |
| 2-7 | C—(A)r—Q | CH | CH | CH₂ | 1 | Q48 | 3- | — | — | — | mp |
| | | | | | | | (trifluoromethyl)phenyl | | | | 121-123° C. |
| 2-8 | C—(A)r—Q | CH | CH | CH₂ | 1 | Q48 | 3- | — | — | — | mp 98-101° C. |
| | | | | | | | (trifluoromethyl)phenyl | | | | |
| 2-9 | C—(A)r—Q | CH | CH | CH₂ | 1 | Q48 | 4- | — | — | — | mp |
| | | | | | | | (trifluoromethyl)phenyl | | | | 180-184° C. |
| 2-10 | C—(A)r—Q | CH | CH | CH₂ | 1 | Q48 | 4- | — | — | — | mp |
| | | | | | | | (trifluoromethyl)phenyl | | | | 112-115° C. |
| 2-11 | C—(A)r—Q | CH | CH | CH₂ | 1 | Q48 | 3,5- | — | — | — | mp 84-87° C. |
| | | | | | | | bis(trifluoromethyl)phenyl | | | | |
| 2-12 | C—(A)r—Q | CH | CH | CH₂ | 1 | Q48 | 3,5- | — | — | — | mp 99-102° C. |
| | | | | | | | bis(trifluoromethyl)phenyl | | | | |
| 2-13 | C—(A)r—Q | CH | CH | CH₂ | 1 | Q8 | C₂F₅ | C₂F₅ | H | — | NMR |
| 2-14 | C—(A)r—Q | CH | CH | CH₂ | 1 | Q8 | C₂F₅ | C₂F₅ | H | — | NMR |
| 2-15 | C—(A)r—Q | CH | CH | CH₂ | 1 | Q34 | C₂F₅ | CF₃ | — | — | NMR |
| 2-16 | C—(A)r—Q | CH | CH | CH₂ | 1 | Q8 | C₂F₅ | I | H | — | |
| 2-17 | C—(A)r—Q | CH | CH | CH₂ | 1 | Q8 | CF₃ | H | H | — | |
| 2-18 | C—(A)r—Q | CH | CH | CH₂ | 1 | Q8 | CF₃ | H | CF₃ | — | |
| 2-19 | C—(A)r—Q | CH | CH | CH₂ | 1 | Q34 | CF₃ | CF₃ | — | — | |
| 2-20 | C—(A)r—Q | CH | CH | CH₂ | 1 | Q34 | CF₃ | CF₃ | — | — | |
| 2-21 | C—(A)r—Q | CH | CH | CH₂ | 1 | Q34 | CF₃ | CF₃ | — | — | |
| 2-22 | C—(A)r—Q | CH | CH | CH₂ | 1 | Q34 | CF₃ | CF₃ | — | — | |
| 2-23 | C—(A)r—Q | CH | CH | CH₂ | 1 | Q34 | CF₃ | CF₃ | — | — | |
| 2-24 | C—(A)r—Q | CH | CH | CH₂ | 1 | Q34 | CF₃ | CF₃ | — | — | |

TABLE 3

| No. | R¹ | R² | R³ | W¹ | W² | W³ | W⁴ | W⁵ | W⁶ | W⁷ |
|---|---|---|---|---|---|---|---|---|---|---|
| 3-1 | CH₃ | H | H | CH | C—Cl | CH | C—Cl | C—(A)r—Q | CH | CH |
| 3-2 | CH₃ | H | H | CH | C—Cl | CH | C—Cl | C—(A)r—Q | CH | CH |
| 3-3 | CH₃ | H | H | CH | C—Cl | CH | C—Cl | C—(A)r—Q | CH | CH |
| 3-4 | CH(CH₃)₂ | H | H | C—Cl | CH | CH | CH | C—(A)r—Q | CH | C—CH₃ |
| 3-5 | CH(CH₃)₂ | H | H | C—Cl | CH | CH | CH | C—(A)r—Q | CH | C—CH₃ |
| 3-6 | CH(CH₃)₂ | H | H | C—Cl | CH | CH | CH | C—(A)r—Q | CH | C—CH₃ |
| 3-7 | CH(CH₃)CH₂SCH₃ (S)-isomer | H | H | C—Cl | CH | CH | CH | C—(A)r—Q | N | C—CH₃ |
| 3-8 | CH(CH₃)CH₂SCH₃ (S)-isomer | H | H | C—Cl | CH | CH | CH | C—(A)r—Q | CH | C—CH₃ |
| 3-9 | CH(CH₃)CH₂SCH₃ (S)-isomer | H | H | C—SCH₃ | CH | CH | CH | C—(A)r—Q | CH | C—CH₃ |
| 3-10 | CH(CH₃)CH₂SCH₃ (S)-isomer | H | H | C—SO₂CH₃ | CH | CH | CH | C—(A)r—Q | CH | C—CH₃ |
| 3-11 | CH(CH₃)CH₂SOCH₃ (S)-isomer | H | H | C—SO₂CH₃ | CH | CH | CH | C—(A)r—Q | CH | C—CH₃ |
| 3-12 | CH(CH₃)CH₂SO₂CH₃ (S)-isomer | H | H | C—SO₂CH₃ | CH | CH | CH | C—(A)r—Q | CH | C—CH₃ |
| 3-13 | CH(CH₃)CH₂SCH₃ (S)-isomer | H | H | C—SCH₃ | CH | CH | CH | C—(A)r—Q | CH | CH |
| 3-14 | CH(CH₃)CH₂SCH₃ (S)-isomer | H | H | C—Cl | CH | CH | CH | C—(A)r—Q | CH | CH |
| 3-15 | CH(CH₃)CH₂SCH₃ (S)-isomer | H | H | C—Cl | CH | CH | CH | C—(A)r—Q | CH | CH |

| | | | | | | | | | | Physical |
|---|---|---|---|---|---|---|---|---|---|---|
| No. | W⁸ | W⁹ | A | r | Q | R⁴ | R⁵ | R⁶ | R⁷ | property |
| 3-1 | C—Cl | CH | CH₂ | 1 | Q8 | CF₃ | H | H | — | |
| 3-2 | C—Cl | CH | CH₂ | 1 | Q34 | CF₃ | CF₃ | — | — | logP (acid) 3.84 |
| 3-3 | C—Cl | CH | CH₂ | 1 | Q48 | 3- | — | — | — | |
| | | | | | | (trifluoromethyl)phenyl | | | | |
| 3-4 | CH | CH | CH₂ | 1 | Q8 | CF₃ | H | H | — | |
| 3-5 | CH | CH | CH₂ | 1 | Q34 | CF₃ | CF₃ | — | — | |
| 3-6 | CH | CH | CH₂ | 1 | Q48 | 3- | — | — | — | |
| | | | | | | (trifluoromethyl)phenyl | | | | |
| 3-7 | C—F | CH | CH₂ | 1 | Q8 | CF₃ | H | CF₃ | — | logP (acid) 4.28 |
| 3-8 | C—F | CH | CH₂ | 1 | Q34 | CF₃ | CF₃ | — | — | logP (acid) 4.13 |
| 3-9 | CH | CH | CH₂ | 1 | Q34 | CF₃ | CF₃ | — | — | mp 185° C. |
| 3-10 | CH | CH | CH₂ | 1 | Q34 | CF₃ | CF₃ | — | — | |
| 3-11 | CH | CH | CH₂ | 1 | Q34 | CF₃ | CF₃ | — | — | |
| 3-12 | CH | CH | CH₂ | 1 | Q34 | CF₃ | CF₃ | — | — | |
| 3-13 | C—Cl | CH | CH₂ | 1 | Q34 | CF₃ | CF₃ | — | — | mp 149-152° C. |
| 3-14 | C—Cl | CH | — | 0 | Q8 | CF₃ | H | H | — | |
| 3-15 | C—Cl | CH | — | 0 | Q8 | CF₃ | CF₃ | H | — | |

TABLE 4

| No. | R¹ | R² | R³ | W¹ | W² | W³ | W⁴ | W⁵ | W⁶ | W⁷ | W⁸ |
|---|---|---|---|---|---|---|---|---|---|---|---|
| 4-1 | CH₃ | H | H | CH | C—Cl | CH | C—Cl | CH | C—(A)r—Q | CH | C—Cl |
| 4-2 | CH(CH₃)₂ | H | H | C—Cl | CH | CH | CH | C—CH₃ | C—(A)r—Q | CH | C—Cl |
| 4-3 | CH(CH₃)CH₂SCH₃ (S)-isomer | H | H | C—Cl | CH | CH | CH | C—CH₃ | C—(A)r—Q | CH | CH |
| 4-4 | CH(CH₃)CH₂SCH₃ (S)-isomer | H | H | C—Cl | CH | CH | CH | C—CH₃ | C—(A)r—Q | CH | CH |
| 4-5 | CH(CH₃)CH₂SCH₃ (S)-isomer | H | H | C—Cl | CH | CH | CH | C—CH₃ | C—(A)r—Q | CH | CH |
| 4-6 | CH(CH₃)CH₂SCH₃ (S)-isomer | H | H | C—Cl | CH | CH | CH | C—CH₃ | C—(A)r—Q | CH | CH |
| 4-7 | CH(CH₃)CH₂SCH₃ (S)-isomer | H | H | C—Cl | CH | CH | CH | C—CH₃ | C—(A)r—Q | CH | CH |
| 4-8 | CH(CH₃)CH₂SCH₃ (S)-isomer | H | H | C—Cl | CH | CH | CH | C—CH₃ | C—(A)r—Q | CH | CH |
| 4-9 | CH(CH₃)CH₂SCH₃ (S)-isomer | H | H | C—Cl | CH | CH | CH | C—CH₃ | C—(A)r—Q | CH | CH |
| 4-10 | CH(CH₃)CH₂SCH₃ (S)-isomer | H | H | C—Cl | CH | CH | CH | C—CH₃ | C—(A)r—Q | CH | CH |
| 4-11 | CH(CH₃)CH₂SCH₃ (S)-isomer | H | H | C—Cl | CH | CH | CH | C—CH₃ | C—(A)r—Q | CH | CH |
| 4-12 | CH(CH₃)CH₂SCH₃ (S)-isomer | H | H | C—Cl | CH | CH | CH | C—CH₃ | C—(A)r—Q | CH | CH |
| 4-13 | CH(CH₃)CH₂SCH₃ (S)-isomer | H | H | C—Cl | CH | CH | CH | C—CH₃ | C—(A)r—Q | CH | CH |
| 4-14 | CH(CH₃)CH₂SCH₃ (S)-isomer | H | H | C—Cl | CH | CH | CH | C—CH₃ | C—(A)r—Q | CH | CH |
| 4-15 | CH(CH₃)CH₂SCH₃ (S)-isomer | H | H | C—Cl | CH | CH | CH | C—CH₃ | C—(A)r—Q | CH | CH |
| 4-16 | CH(CH₃)CH₂SCH₃ (S)-isomer | H | H | C—Cl | CH | CH | CH | CH | C—(A)r—Q | CH | CH |
| 4-17 | CH(CH₃)CH₂SCH₃ (S)-isomer | H | H | C—Cl | CH | CH | CH | CH | C—(A)r—Q | CH | CH |
| 4-18 | CH(CH₃)CH₂SCH₃ (S)-isomer | H | H | C—SCH₃ | CH | CH | CH | C—CH₃ | C—(A)r—Q | CH | CH |
| 4-19 | CH(CH₃)CH₂SCH₃ (S)-isomer | H | H | C—SCH₃ | CH | CH | CH | C—CH₃ | C—(A)r—Q | CH | CH |
| 4-20 | CH(CH₃)CH₂SCH₃ (S)-isomer | H | H | C—SCH₃ | CH | CH | CH | CH | C—(A)r—Q | C—CH₃ | CH |
| 4-21 | CH(CH₃)CH₂SCH₃ (S)- | H | H | C—Cl | CH | CH | CH | N | C—(A)r—Q | CH | CH |
| | isomer | | | | | | | | | | |
| 4-22 | CH(CH₃)CH₂SCH₃ (S)- | H | H | C—Cl | CH | CH | CH | CH | C—(A)r—Q | N | CH |
| | isomer | | | | | | | | | | |
| 4-23 | CH(CH₃)CH₂SCH₃ (S)- | H | H | C—Cl | CH | CH | CH | N | C—(A)r—Q | CH | C—CH₃ |
| | isomer | | | | | | | | | | |
| 4-24 | CH(CH₃)CH₂SCH₃ (S)- | H | H | C—Cl | CH | CH | CH | CH | C—(A)r—Q | CH | CH |
| | isomer | | | | | | | | | | |
| 4-25 | CH(CH₃)CH₂SCH₃ (S)- | H | H | C—Cl | CH | CH | CH | C—CH₃ | C—(A)r—Q | CH | CH |
| | isomer | | | | | | | | | | |
| 4-26 | CH(CH₃)CH₂SCH₃ (S)- | H | H | C—Cl | CH | CH | CH | C—CH₃ | C—(A)r—Q | CH | CH |
| | isomer | | | | | | | | | | |
| 4-27 | CH(CH₃)CH₂SCH₃ (S)- | H | H | C—Cl | CH | CH | CH | C—CH₃ | C—(A)r—Q | CH | CH |
| | isomer | | | | | | | | | | |
| 4-28 | CH(CH₃)CH₂SCH₃ (S)- | H | H | C—Cl | CH | CH | CH | C—CH₃ | C—(A)r—Q | CH | CH |
| | isomer | | | | | | | | | | |
| 4-29 | CH(CH₃)CH₂SCH₃ (S)- | H | H | C—Cl | CH | CH | CH | C—CH₃ | C—(A)r—Q | CH | CH |
| | isomer | | | | | | | | | | |
| 4-30 | CH(CH₃)CH₂SCH₃ (S)- | H | H | C—Cl | CH | CH | CH | C—CH₃ | C—(A)r—Q | CH | CH |
| | isomer | | | | | | | | | | |
| 4-31 | CH(CH₃)CH₂SCH₃ (S)- | H | H | C—Cl | CH | CH | CH | C—CH₃ | C—(A)r—Q | CH | CH |
| | isomer | | | | | | | | | | |
| 4-32 | CH(CH₃)CH₂SCH₃ (S)- | H | H | C—Cl | CH | CH | CH | C—CH₃ | C—(A)r—Q | CH | CH |
| | isomer | | | | | | | | | | |
| 4-33 | C(CH₃)₂CH₂SCH₃ | H | H | C—I | CH | CH | CH | C—CH₃ | C—(A)r—Q | CH | CH |

| | | | | | | | | | Physical |
|---|---|---|---|---|---|---|---|---|---|
| No. | W⁹ | A | r | Q | R⁴ | R⁵ | R⁶ | R⁷ | property |
| 4-1 | CH | CH₂ | 1 | Q34 | CF₃ | CF₃ | — | — | logP (acid) 3.32 |
| 4-2 | CH | CH₂ | 1 | Q8 | C₂F₅ | H | H | — | |
| 4-3 | CH | CH₂ | 1 | Q8 | CF₃ | H | CF₃ | — | |
| 4-4 | CH | CH₂ | 1 | Q8 | C₂F₅ | H | C₂F₅ | — | |
| 4-5 | CH | CH₂ | 1 | Q8 | C₂F₅ | 4- | H | — | NMR |
| | | | | | | (trifluoromethyl)phenyl | | | |
| 4-6 | CH | CH₂ | 1 | Q8 | C₂F₅ | C₂F₅ | H | — | logP (acid) 4.52 |
| 4-7 | CH | CH₂ | 1 | Q8 | C₂F₅ | I | H | — | NMR |
| 4-8 | CH | CH₂ | 1 | Q34 | CF₃ | CF₃ | — | — | |
| 4-9 | CH | CH₂ | 1 | Q34 | C₂F₅ | 4-chlorophenyl | — | — | logP (acid) 4.32 |
| 4-10 | CH | CH₂ | 1 | Q34 | C₂F₅ | 4- | — | — | logP (acid) 4.44 |
| | | | | | | (trifluoromethyl)phenyl | | | |
| 4-11 | CH | CH₂ | 1 | Q34 | C₂F₅ | 6-chloro-pyridin- | — | — | logP (acid) 3.8 |
| | | | | | | 3-yl | | | |
| 4-12 | CH | CH₂ | 1 | Q34 | C₂F₅ | [3,5- | — | — | NMR |
| | | | | | | bis(trifluoromethyl)- | | | |
| | | | | | | pyrazol-1-yl]- | | | |
| | | | | | | methyl | | | |
| 4-13 | CH | CH₂ | 1 | Q34 | C₂F₅ | 3-chloro-5- | — | — | NMR |
| | | | | | | (trifluoromethyl)- | | | |
| | | | | | | pyridin-2-yl | | | |
| 4-14 | CH | CH₂ | 1 | Q34 | C₂F₅ | 4-[(methylthio)methyl]phenyl | — | — | NMR |
| 4-15 | CH | CH₂ | 1 | Q34 | C₂F₅ | 4-chloro-thiophen- | — | — | NMR |
| | | | | | | 2-yl | | | |
| 4-16 | CH | CH₂ | 1 | Q47 | 4- | — | — | — | logP (acid) 3.86 |
| | | | | | (trifluoromethyl)phenyl | | | | |
| 4-17 | CH | CH₂ | 1 | Q48 | 4- | — | — | — | |
| | | | | | (trifluoromethyl)phenyl | | | | |
| 4-18 | CH | CH₂ | 1 | Q34 | CF₃ | CF₃ | — | — | mp 86-89° C. |
| 4-19 | CH | CH₂ | 1 | Q8 | C₂F₅ | H | C₂F₅ | — | NMR |
| 4-20 | CH | CH₂ | 1 | Q8 | CF₃ | H | H | — | NMR |
| 4-21 | CH | — | 0 | Q8 | CF₃ | H | CF₃ | — | |
| 4-22 | CH | — | 0 | Q8 | CF₃ | H | CF₃ | — | |
| 4-23 | N | — | 0 | Q8 | 4-chlorophenyl | H | CF₃ | — | |
| 4-24 | C—CO₂CH₃ | CH₂ | 1 | Q34 | CF₃ | CF₃ | — | — | NMR |
| 4-25 | CH | CH₂ | 1 | Q34 | C₂F₅ | 2-cyanophenyl | — | — | |
| 4-26 | CH | CH₂ | 1 | Q34 | C₂F₅ | 3-methylthiophenyl | — | — | |
| 4-27 | CH | CH₂ | 1 | Q34 | C₂F₅ | 3- | — | — | |
| | | | | | | (trifluoromethylthio)phenyl | | | |
| 4-28 | CH | CH₂ | 1 | Q34 | C₂F₅ | 4-ethylphenyl | — | — | |
| 4-29 | CH | CH₂ | 1 | Q34 | C₂F₅ | 4-vinylphenyl | — | — | |
| 4-30 | CH | CH₂ | 1 | Q34 | C₂F₅ | 4-acety | — | — | |
| | | | | | | phenyl | | | |
| 4-31 | CH | CH₂ | 1 | Q34 | C₂F₅ | 4-methoxycarbonyl | — | — | |
| | | | | | | phenyl | | | |
| 4-32 | CH | CH₂ | 1 | Q34 | C₂F₅ | 4- | — | — | |
| | | | | | | (trifluoromethoxy)phenyl | | | |
| 4-33 | CH | CH₂ | 1 | Q8 | CF₃ | CF₃ | — | — | mp 181-182° C. |

TABLE 5

| No. | R¹ | R² | R³ | W¹ | W² | W³ | W⁴ | W⁵ | W⁶ |
|---|---|---|---|---|---|---|---|---|---|
| 5-1 | CH(CH₃)₂ | H | H | CH | CH | CH | CH | C—Cl | CH |
| 5-2 | CH(CH₃)₂ | H | H | CH | CH | CH | CH | C—Cl | CH |
| 5-3 | CH(CH₃)CH₂SCH₃ (S)-isomer | H | H | C—Cl | CH | CH | CH | C—Cl | CH |
| 5-4 | CH(CH₃)CH₂SCH₃ (S)-isomer | H | H | C—Cl | CH | CH | CH | C—Br | CH |
| 5-5 | CH(CH₃)CH₂SCH₃ (S)-isomer | H | H | C—Cl | CH | CH | CH | C—I | CH |
| 5-6 | CH(CH₃)CH₂SCH₃ (S)-isomer | H | H | C—Cl | CH | CH | CH | C—F | CH |
| 5-7 | CH(CH₃)CH₂SOCH₃ (S)-isomer | H | H | C—Cl | CH | CH | CH | C—F | CH |
| 5-8 | CH(CH₃)CH₂SO₂CH₃ (S)-isomer | H | H | C—Cl | CH | CH | CH | C—F | CH |
| 5-9 | CH(CH₃)CH₂SCH₃ (S)-isomer | H | H | C—Cl | CH | CH | CH | C—CH₃ | CH |
| 5-10 | CH(CH₃)CH₂SCH₃ (S)-isomer | H | H | C—Cl | CH | CH | CH | C—CH₃ | CH |
| 5-11 | CH(CH₃)CH₂SCH₃ (S)-isomer | H | H | C—Cl | CH | CH | CH | C—CH₃ | CH |
| 5-12 | CH(CH₃)CH₂SCH₃ (S)-isomer | H | H | C—Cl | CH | CH | CH | C—CH₃ | CH |
| 5-13 | CH(CH₃)CH₂SCH₃ (S)-isomer | H | H | C—Cl | CH | CH | CH | C—CH₃ | CH |
| 5-14 | CH(CH₃)CH₂SCH₃ (S)-isomer | H | H | C—Cl | CH | CH | CH | C—Cl | CH |
| 5-15 | CH(CH₃)CH₂SCH₃ (S)-isomer | H | H | C—Cl | CH | CH | CH | C—Br | CH |
| 5-16 | CH(CH₃)CH₂SCH₃ (S)-isomer | H | H | C—I | CH | CH | CH | C—Cl | CH |
| 5-17 | CH(CH₃)CH₂SCH₃ (S)-isomer | H | H | C—I | CH | CH | CH | C—Br | CH |
| 5-18 | CH(CH₃)CH₂SCH₃ (S)-isomer | H | H | C—I | CH | CH | CH | C—Cl | CH |
| 5-19 | CH(CH₃)CH₂SOCH₃ (S)-isomer | H | H | C—I | CH | CH | CH | C—Cl | CH |
| 5-20 | CH(CH₃)CH₂SO₂CH₃ (S)-isomer | H | H | C—I | CH | CH | CH | C—Cl | CH |
| 5-21 | CH(CH₃)CH₂SCH₃ (S)-isomer | H | H | C—Cl | CH | CH | CH | C—CH₃ | CH |
| 5-22 | CH(CH₃)CH₂SCH₃ (S)-isomer | H | H | C—Cl | CH | CH | CH | C—CH₃ | CH |
| 5-23 | CH(CH₃)CH₂SCH₃ (S)-isomer | H | H | C—I | CH | CH | CH | C—Br | CH |
| 5-24 | CH(CH₃)CH₂SCH₃ (S)-isomer | H | H | C—SCH₃ | CH | CH | CH | C—CH₃ | CH |

| | | | | | | | | | | | Physical |
|---|---|---|---|---|---|---|---|---|---|---|---|
| No. | W⁷ | W⁸ | W⁹ | A | r | Q | R⁴ | R⁵ | R⁶ | R⁷ | property |
| 5-1 | C—(A)r—Q | CH | C—Cl | CH₂ | 1 | Q8 | CF₃ | H | CF₃ | — | |
| 5-2 | C—(A)r—Q | CH | C—Cl | CH₂ | 1 | Q34 | CF₃ | CF₃ | — | — | |
| 5-3 | C—(A)r—Q | CH | C—Cl | CH₂ | 1 | Q8 | CF₃ | H | CF₃ | — | NMR |
| 5-4 | C—(A)r—Q | CH | C—Br | CH₂ | 1 | Q8 | CF₃ | H | CF₃ | — | NMR |
| 5-5 | C—(A)r—Q | CH | C—I | CH₂ | 1 | Q8 | C₂F₅ | C₂F₅ | H | — | NMR |
| 5-6 | C—(A)r—Q | CH | C—I | CH₂ | 1 | Q8 | C₂F₅ | C₂F₅ | H | — | NMR |
| 5-7 | C—(A)r—Q | CH | C—I | CH₂ | 1 | Q8 | C₂F₅ | C₂F₅ | H | — | |
| 5-8 | C—(A)r—Q | CH | C—I | CH₂ | 1 | Q8 | C₂F₅ | C₂F₅ | H | — | |
| 5-9 | C—(A)r—Q | CH | C—CH₃ | CH₂ | 1 | Q8 | C₂F₅ | H | H | — | |
| 5-10 | C—(A)r—Q | CH | C—CH₃ | CH₂ | 1 | Q8 | C₂F₅ | H | C₂F₅ | — | NMR |
| 5-11 | C—(A)r—Q | CH | C—CH₃ | CH₂ | 1 | Q34 | CF₃ | CF₃ | — | — | |
| 5-12 | C—(A)r—Q | CH | C—CH₃ | CH₂ | 1 | Q34 | C₂F₅ | CF₃ | — | — | |
| 5-13 | C—(A)r—Q | CH | C—CH₃ | CH₂ | 1 | Q34 | C₂F₅ | C₂F₅ | — | — | |
| 5-14 | C—(A)r—Q | CH | C—Cl | CH₂ | 1 | Q34 | CF₃ | CF₃ | — | — | NMR |
| 5-15 | C—(A)r—Q | CH | C—Br | CH₂ | 1 | Q34 | CF₃ | CF₃ | — | — | NMR |
| 5-16 | C—(A)r—Q | CH | C—Cl | CH₂ | 1 | Q8 | CF₃ | H | CF₃ | — | NMR |
| 5-17 | C—(A)r—Q | CH | C—Br | CH₂ | 1 | Q8 | CF₃ | H | CF₃ | — | NMR |
| 5-18 | C—(A)r—Q | CH | C—Cl | CH₂ | 1 | Q34 | CF₃ | CF₃ | — | — | NMR |
| 5-19 | C—(A)r—Q | CH | C—Cl | CH₂ | 1 | Q34 | CF₃ | CF₃ | — | — | |
| 5-20 | C—(A)r—Q | CH | C—Cl | CH₂ | 1 | Q34 | CF₃ | CF₃ | — | — | |
| 5-21 | C—(A)r—Q | CH | C—Cl | CH₂ | 1 | Q8 | CF₃ | H | CF₃ | — | NMR |
| 5-22 | C—(A)r—Q | CH | C—Cl | CH₂ | 1 | Q34 | CF₃ | CF₃ | — | — | NMR |
| 5-23 | C—(A)r—Q | CH | C—Br | CH₂ | 1 | Q34 | CF₃ | CF₃ | — | — | NMR |
| 5-24 | C—(A)r—Q | CH | C—Cl | CH₂ | 1 | Q34 | CF₃ | CF₃ | — | — | NMR |

TABLE 6

| No. | R¹ | R² | R³ | W¹ | W² | W³ | W⁴ | W⁵ | W⁶ | W⁷ | W⁸ | W⁹ | A | r | Q | R⁴ | R⁵ | R⁶ | R⁷ | Physical property |
|---|---|---|---|---|---|---|---|---|---|---|---|---|---|---|---|---|---|---|---|---|
| 6-1 | CH(CH₃)CH₂SCH₃ (S)-isomer | H | H | C—Cl | CH | CH | CH | C—CH₃ | C—F | C—(A)r—Q | CH | CH | CH₂ | 1 | Q8 | CF₃ | H | H | — | |
| 6-2 | CH(CH₃)CH₂SCH₃ (S)-isomer | H | H | C—Cl | CH | CH | CH | C—CH₃ | C—F | C—(A)r—Q | CH | CH | CH₂ | 1 | Q34 | CF₃ | H | — | — | |
| 6-3 | CH(CH₃)CH₂SCH₃ (S)-isomer | H | H | C—Br | CH | CH | CH | C—CH₃ | C—F | C—(A)r—Q | CH | CH | CH₂ | 1 | Q8 | CF₃ | H | H | — | |
| 6-4 | CH(CH₃)CH₂SCH₃ (S)-isomer | H | H | C—Br | CH | CH | CH | C—CH₃ | C—F | C—(A)r—Q | CH | CH | CH₂ | 1 | Q34 | CF₃ | H | — | — | |
| 6-5 | CH(CH₃)CH₂SCH₃ (S)-isomer | H | H | C—I | CH | CH | CH | C—CH₃ | C—F | C—(A)r—Q | CH | CH | CH₂ | 1 | Q8 | CF₃ | H | H | — | |
| 6-6 | CH(CH₃)CH₂SCH₃ (S)-isomer | H | H | C—I | CH | CH | CH | C—CH₃ | C—F | C—(A)r—Q | CH | CH | CH₂ | 1 | Q34 | CF₃ | H | — | — | |
| 6-7 | CH(CH₃)CH₂SCH₃ (S)-isomer | H | H | C—SCH₃ | CH | CH | CH | C—CH₃ | C—F | C—(A)r—Q | CH | CH | CH₂ | 1 | Q8 | CF₃ | H | H | — | |
| 6-8 | CH(CH₃)CH₂SCH₃ (S)-isomer | H | H | C—SCH₃ | CH | CH | CH | C—CH₃ | C—F | C—(A)r—Q | CH | CH | CH₂ | 1 | Q34 | CF₃ | H | — | — | |
| 6-9 | CH(CH₃)CH₂SCH₃ (S)-isomer | H | H | C—SO₂CH₃ | CH | CH | CH | C—CH₃ | C—F | C—(A)r—Q | CH | CH | CH₂ | 1 | Q8 | CF₃ | H | H | — | |
| 6-10 | CH(CH₃)CH₂SCH₃ (S)-isomer | H | H | C—SO₂CH₃ | CH | CH | CH | C—CH₃ | C—F | C—(A)r—Q | CH | CH | CH₂ | 1 | Q34 | CF₃ | H | — | — | |
| 6-11 | CH(CH₃)CH₂SCH₃ (S)-isomer | H | H | C—Cl | CH | CH | CH | C—CH₃ | C—F | C—(A)r—Q | CH | CH | CH₂ | 1 | Q8 | CF₃ | H | CF₃ | — | |
| 6-12 | CH(CH₃)CH₂SOCH₃ (S)-isomer | H | H | C—Cl | CH | CH | CH | C—CH₃ | C—F | C—(A)r—Q | CH | CH | CH₂ | 1 | Q8 | CF₃ | H | CF₃ | — | |
| 6-13 | CH(CH₃)CH₂SO₂CH₃ (S)-isomer | H | H | C—Cl | CH | CH | CH | C—CH₃ | C—F | C—(A)r—Q | CH | CH | CH₂ | 1 | Q8 | CF₃ | H | CF₃ | — | |
| 6-14 | CH(CH₃)CH₂SCH₃ (S)-isomer | H | H | C—Cl | CH | CH | CH | C—CH₃ | C—F | C—(A)r—Q | CH | CH | CH₂ | 1 | Q34 | CF₃ | CF₃ | — | — | NMR |
| 6-15 | CH(CH₃)CH₂SOCH₃ (S)-isomer | H | H | C—Cl | CH | CH | CH | C—CH₃ | C—F | C—(A)r—Q | CH | CH | CH₂ | 1 | Q34 | CF₃ | CF₃ | — | — | NMR |
| 6-16 | CH(CH₃)CH₂SO₂CH₃ (S)-isomer | H | H | C—Cl | CH | CH | CH | C—CH₃ | C—F | C—(A)r—Q | CH | CH | CH₂ | 1 | Q34 | CF₃ | CF₃ | — | — | NMR |
| 6-17 | CH(CH₃)CH₂SCH₃ (S)-isomer | H | H | C—Br | CH | CH | CH | C—CH₃ | C—F | C—(A)r—Q | CH | CH | CH₂ | 1 | Q8 | CF₃ | H | CF₃ | — | |
| 6-18 | CH(CH₃)CH₂SOCH₃ (S)-isomer | H | H | C—Br | CH | CH | CH | C—CH₃ | C—F | C—(A)r—Q | CH | CH | CH₂ | 1 | Q8 | CF₃ | H | CF₃ | — | |
| 6-19 | CH(CH₃)CH₂SO₂CH₃ (S)-isomer | H | H | C—Br | CH | CH | CH | C—CH₃ | C—F | C—(A)r—Q | CH | CH | CH₂ | 1 | Q8 | CF₃ | H | CF₃ | — | |
| 6-20 | CH(CH₃)CH₂SCH₃ (S)-isomer | H | H | C—Br | CH | CH | CH | C—CH₃ | C—F | C—(A)r—Q | CH | CH | CH₂ | 1 | Q34 | CF₃ | CF₃ | — | — | NMR |
| 6-21 | CH(CH₃)CH₂SOCH₃ (S)-isomer | H | H | C—Br | CH | CH | CH | C—CH₃ | C—F | C—(A)r—Q | CH | CH | CH₂ | 1 | Q34 | CF₃ | CF₃ | — | — | |
| 6-22 | CH(CH₃)CH₂SO₂CH₃ (S)-isomer | H | H | C—Br | CH | CH | CH | C—CH₃ | C—F | C—(A)r—Q | CH | CH | CH₂ | 1 | Q34 | CF₃ | CF₃ | — | — | |
| 6-23 | CH(CH₃)CH₂SCH₃ (S)-isomer | H | H | C—I | CH | CH | CH | C—CH₃ | C—F | C—(A)r—Q | CH | CH | CH₂ | 1 | Q8 | CF₃ | H | CF₃ | — | |
| 6-24 | CH(CH₃)CH₂SOCH₃ (S)-isomer | H | H | C—I | CH | CH | CH | C—CH₃ | C—F | C—(A)r—Q | CH | CH | CH₂ | 1 | Q8 | CF₃ | H | CF₃ | — | |
| 6-25 | CH(CH₃)CH₂SO₂CH₃ (S)-isomer | H | H | C—I | CH | CH | CH | C—CH₃ | C—F | C—(A)r—Q | CH | CH | CH₂ | 1 | Q8 | CF₃ | H | CF₃ | — | |
| 6-26 | CH(CH₃)CH₂SCH₃ (S)-isomer | H | H | C—I | CH | CH | CH | C—CH₃ | C—F | C—(A)r—Q | CH | CH | CH₂ | 1 | Q34 | CF₃ | CF₃ | — | — | NMR |
| 6-27 | CH(CH₃)CH₂SOCH₃ (S)-isomer | H | H | C—I | CH | CH | CH | C—CH₃ | C—F | C—(A)r—Q | CH | CH | CH₂ | 1 | Q34 | CF₃ | CF₃ | — | — | |
| 6-28 | CH(CH₃)CH₂SO₂CH₃ (S)-isomer | H | H | C—I | CH | CH | CH | C—CH₃ | C—F | C—(A)r—Q | CH | CH | CH₂ | 1 | Q34 | CF₃ | CF₃ | — | — | |
| 6-29 | CH(CH₃)CH₂SCH₃ (S)-isomer | H | H | C—SCH₃ | CH | CH | CH | C—CH₃ | C—F | C—(A)r—Q | CH | CH | CH₂ | 1 | Q8 | CF₃ | H | CF₃ | — | |
| 6-30 | CH(CH₃)CH₂SCH₃ (S)-isomer | H | H | C—SCH₃ | CH | CH | CH | C—CH₃ | C—F | C—(A)r—Q | CH | CH | CH₂ | 1 | Q34 | CF₃ | CF₃ | — | — | NMR |
| 6-31 | CH(CH₃)CH₂SCH₃ (S)-isomer | H | H | C—SO₂CH₃ | CH | CH | CH | C—CH₃ | C—F | C—(A)r—Q | CH | CH | CH₂ | 1 | Q8 | CF₃ | H | CF₃ | — | |
| 6-32 | CH(CH₃)CH₂SOCH₃ (S)-isomer | H | H | C—SO₂CH₃ | CH | CH | CH | C—CH₃ | C—F | C—(A)r—Q | CH | CH | CH₂ | 1 | Q8 | CF₃ | H | CF₃ | — | |
| 6-33 | CH(CH₃)CH₂SO₂CH₃ (S)-isomer | H | H | C—SO₂CH₃ | CH | CH | CH | C—CH₃ | C—F | C—(A)r—Q | CH | CH | CH₂ | 1 | Q8 | CF₃ | H | CF₃ | — | |
| 6-34 | CH(CH₃)CH₂SCH₃ (S)-isomer | H | H | C—SO₂CH₃ | CH | CH | CH | C—CH₃ | C—F | C—(A)r—Q | CH | CH | CH₂ | 1 | Q34 | CF₃ | CF₃ | — | — | NMR |
| 6-35 | CH(CH₃)CH₂SOCH₃ (S)-isomer | H | H | C—SO₂CH₃ | CH | CH | CH | C—CH₃ | C—F | C—(A)r—Q | CH | CH | CH₂ | 1 | Q34 | CF₃ | CF₃ | — | — | NMR |
| 6-36 | CH(CH₃)CH₂SO₂CH₃ (S)-isomer | H | H | C—SO₂CH₃ | CH | CH | CH | C—CH₃ | C—F | C—(A)r—Q | CH | CH | CH₂ | 1 | Q34 | CF₃ | CF₃ | — | — | NMR |
| 6-37 | CH(CH₃)CH₂SCH₃ (S)-isomer | H | H | C—Cl | CH | CH | CH | C—CH₃ | C—F | C—(A)r—Q | CH | CH | CH₂ | 1 | Q34 | CF₂CHF₂ | CF₃ | — | — | |
| 6-38 | CH(CH₃)CH₂SCH₃ (S)-isomer | H | H | C—Cl | CH | CH | CH | C—CH₃ | C—F | C—(A)r—Q | CH | CH | CH₂ | 1 | Q34 | CF₃ | CF₂CHF₂ | — | — | |
| 6-39 | CH(CH₃)CH₂SCH₃ (S)-isomer | H | H | C—Br | CH | CH | CH | C—CH₃ | C—F | C—(A)r—Q | CH | CH | CH₂ | 1 | Q34 | CF₂CHF₂ | CF₃ | — | — | |
| 6-40 | CH(CH₃)CH₂SCH₃ (S)-isomer | H | H | C—Br | CH | CH | CH | C—CH₃ | C—F | C—(A)r—Q | CH | CH | CH₂ | 1 | Q34 | CF₃ | CF₂CHF₂ | — | — | |
| 6-41 | CH(CH₃)CH₂SCH₃ (S)-isomer | H | H | C—I | CH | CH | CH | C—CH₃ | C—F | C—(A)r—Q | CH | CH | CH₂ | 1 | Q34 | CF₂CHF₂ | CF₃ | — | — | |
| 6-42 | CH(CH₃)CH₂SCH₃ (S)-isomer | H | H | C—I | CH | CH | CH | C—CH₃ | C—F | C—(A)r—Q | CH | CH | CH₂ | 1 | Q34 | CF₃ | CF₂CHF₂ | — | — | |
| 6-43 | CH(CH₃)CH₂SCH₃ (S)-isomer | H | H | C—SCH₃ | CH | CH | CH | C—CH₃ | C—F | C—(A)r—Q | CH | CH | CH₂ | 1 | Q34 | CF₂CHF₂ | CF₃ | — | — | |
| 6-44 | CH(CH₃)CH₂SCH₃ (S)-isomer | H | H | C—SCH₃ | CH | CH | CH | C—CH₃ | C—F | C—(A)r—Q | CH | CH | CH₂ | 1 | Q34 | CF₃ | CF₂CHF₂ | — | — | |
| 6-45 | CH(CH₃)CH₂SCH₃ (S)-isomer | H | H | C—SO₂CH₃ | CH | CH | CH | C—CH₃ | C—F | C—(A)r—Q | CH | CH | CH₂ | 1 | Q34 | CF₂CHF₂ | CF₃ | — | — | |
| 6-46 | CH(CH₃)CH₂SCH₃ (S)-isomer | H | H | C—SO₂CH₃ | CH | CH | CH | C—CH₃ | C—F | C—(A)r—Q | CH | CH | CH₂ | 1 | Q34 | CF₃ | CF₂CHF₂ | — | — | |
| 6-47 | CH(CH₃)CH₂SCH₃ (S)-isomer | H | H | C—Cl | CH | CH | CH | C—CH₃ | C—F | C—(A)r—Q | CH | CH | CH₂ | 1 | Q34 | C₂F₅ | CF₃ | — | — | |
| 6-48 | CH(CH₃)CH₂SCH₃ (S)-isomer | H | H | C—Br | CH | CH | CH | C—CH₃ | C—F | C—(A)r—Q | CH | CH | CH₂ | 1 | Q34 | C₂F₅ | CF₃ | — | — | |
| 6-49 | CH(CH₃)CH₂SCH₃ (S)-isomer | H | H | C—I | CH | CH | CH | C—CH₃ | C—F | C—(A)r—Q | CH | CH | CH₂ | 1 | Q34 | C₂F₅ | CF₃ | — | — | |
| 6-50 | CH(CH₃)CH₂SCH₃ (S)-isomer | H | H | C—SCH₃ | CH | CH | CH | C—CH₃ | C—F | C—(A)r—Q | CH | CH | CH₂ | 1 | Q34 | C₂F₅ | CF₃ | — | — | |
| 6-51 | CH(CH₃)CH₂SCH₃ (S)-isomer | H | H | C—SO₂CH₃ | CH | CH | CH | C—CH₃ | C—F | C—(A)r—Q | CH | CH | CH₂ | 1 | Q34 | C₂F₅ | CF₃ | — | — | |
| 6-52 | CH(CH₃)CH₂SCH₃ (S)-isomer | H | H | C—Cl | CH | CH | CH | C—Cl | C—F | C—(A)r—Q | CH | CH | CH₂ | 1 | Q8 | CF₃ | H | H | — | |
| 6-53 | CH(CH₃)CH₂SCH₃ (S)-isomer | H | H | C—Cl | CH | CH | CH | C—Cl | C—F | C—(A)r—Q | CH | CH | CH₂ | 1 | Q34 | CF₃ | H | — | — | |
| 6-54 | CH(CH₃)CH₂SCH₃ (S)-isomer | H | H | C—Br | CH | CH | CH | C—Cl | C—F | C—(A)r—Q | CH | CH | CH₂ | 1 | Q8 | CF₃ | H | H | — | |
| 6-55 | CH(CH₃)CH₂SCH₃ (S)-isomer | H | H | C—Br | CH | CH | CH | C—Cl | C—F | C—(A)r—Q | CH | CH | CH₂ | 1 | Q34 | CF₃ | H | — | — | |
| 6-56 | CH(CH₃)CH₂SCH₃ (S)-isomer | H | H | C—I | CH | CH | CH | C—Cl | C—F | C—(A)r—Q | CH | CH | CH₂ | 1 | Q8 | CF₃ | H | H | — | |
| 6-57 | CH(CH₃)CH₂SCH₃ (S)-isomer | H | H | C—I | CH | CH | CH | C—Cl | C—F | C—(A)r—Q | CH | CH | CH₂ | 1 | Q34 | CF₃ | H | — | — | |
| 6-58 | CH(CH₃)CH₂SCH₃ (S)-isomer | H | H | C—SCH₃ | CH | CH | CH | C—Cl | C—F | C—(A)r—Q | CH | CH | CH₂ | 1 | Q8 | CF₃ | H | H | — | |
| 6-59 | CH(CH₃)CH₂SCH₃ (S)-isomer | H | H | C—SCH₃ | CH | CH | CH | C—Cl | C—F | C—(A)r—Q | CH | CH | CH₂ | 1 | Q34 | CF₃ | H | — | — | |
| 6-60 | CH(CH₃)CH₂SCH₃ (S)-isomer | H | H | C—SO₂CH₃ | CH | CH | CH | C—Cl | C—F | C—(A)r—Q | CH | CH | CH₂ | 1 | Q8 | CF₃ | H | H | — | |
| 6-61 | CH(CH₃)CH₂SCH₃ (S)-isomer | H | H | C—SO₂CH₃ | CH | CH | CH | C—Cl | C—F | C—(A)r—Q | CH | CH | CH₂ | 1 | Q34 | CF₃ | H | — | — | |
| 6-62 | CH(CH₃)CH₂SCH₃ (S)-isomer | H | H | C—Cl | CH | CH | CH | C—Cl | C—F | C—(A)r—Q | CH | CH | CH₂ | 1 | Q8 | CF₃ | H | CF₃ | — | |
| 6-63 | CH(CH₃)CH₂SOCH₃ (S)-isomer | H | H | C—Cl | CH | CH | CH | C—Cl | C—F | C—(A)r—Q | CH | CH | CH₂ | 1 | Q8 | CF₃ | H | CF₃ | — | |
| 6-64 | CH(CH₃)CH₂SO₂CH₃ (S)-isomer | H | H | C—Cl | CH | CH | CH | C—Cl | C—F | C—(A)r—Q | CH | CH | CH₂ | 1 | Q8 | CF₃ | H | CF₃ | — | |
| 6-65 | CH(CH₃)CH₂SCH₃ (S)-isomer | H | H | C—Cl | CH | CH | CH | C—Cl | C—F | C—(A)r—Q | CH | CH | CH₂ | 1 | Q34 | CF₃ | CF₃ | — | — | NMR |
| 6-66 | CH(CH₃)CH₂SOCH₃ (S)-isomer | H | H | C—Cl | CH | CH | CH | C—Cl | C—F | C—(A)r—Q | CH | CH | CH₂ | 1 | Q34 | CF₃ | CF₃ | — | — | |
| 6-67 | CH(CH₃)CH₂SO₂CH₃ (S)-isomer | H | H | C—Cl | CH | CH | CH | C—Cl | C—F | C—(A)r—Q | CH | CH | CH₂ | 1 | Q34 | CF₃ | CF₃ | — | — | |
| 6-68 | CH(CH₃)CH₂SCH₃ (S)-isomer | H | H | C—Br | CH | CH | CH | C—Cl | C—F | C—(A)r—Q | CH | CH | CH₂ | 1 | Q8 | CF₃ | H | CF₃ | — | |
| 6-69 | CH(CH₃)CH₂SOCH₃ (S)-isomer | H | H | C—Br | CH | CH | CH | C—Cl | C—F | C—(A)r—Q | CH | CH | CH₂ | 1 | Q8 | CF₃ | H | CF₃ | — | |
| 6-70 | CH(CH₃)CH₂SO₂CH₃ (S)-isomer | H | H | C—Br | CH | CH | CH | C—Cl | C—F | C—(A)r—Q | CH | CH | CH₂ | 1 | Q8 | CF₃ | H | CF₃ | — | |
| 6-71 | CH(CH₃)CH₂SCH₃ (S)-isomer | H | H | C—Br | CH | CH | CH | C—Cl | C—F | C—(A)r—Q | CH | CH | CH₂ | 1 | Q34 | CF₃ | CF₃ | — | — | |
| 6-72 | CH(CH₃)CH₂SOCH₃ (S)-isomer | H | H | C—Br | CH | CH | CH | C—Cl | C—F | C—(A)r—Q | CH | CH | CH₂ | 1 | Q34 | CF₃ | CF₃ | — | — | |
| 6-73 | CH(CH₃)CH₂SO₂CH₃ (S)-isomer | H | H | C—Br | CH | CH | CH | C—Cl | C—F | C—(A)r—Q | CH | CH | CH₂ | 1 | Q34 | CF₃ | CF₃ | — | — | |
| 6-74 | CH(CH₃)CH₂SCH₃ (S)-isomer | H | H | C—I | CH | CH | CH | C—Cl | C—F | C—(A)r—Q | CH | CH | CH₂ | 1 | Q8 | CF₃ | H | CF₃ | — | |
| 6-75 | CH(CH₃)CH₂SOCH₃ (S)-isomer | H | H | C—I | CH | CH | CH | C—Cl | C—F | C—(A)r—Q | CH | CH | CH₂ | 1 | Q8 | CF₃ | H | CF₃ | — | |
| 6-76 | CH(CH₃)CH₂SO₂CH₃ (S)-isomer | H | H | C—I | CH | CH | CH | C—Cl | C—F | C—(A)r—Q | CH | CH | CH₂ | 1 | Q8 | CF₃ | H | CF₃ | — | |
| 6-77 | CH(CH₃)CH₂SCH₃ (S)-isomer | H | H | C—I | CH | CH | CH | C—Cl | C—F | C—(A)r—Q | CH | CH | CH₂ | 1 | Q34 | CF₃ | CF₃ | — | — | |
| 6-78 | CH(CH₃)CH₂SOCH₃ (S)-isomer | H | H | C—I | CH | CH | CH | C—Cl | C—F | C—(A)r—Q | CH | CH | CH₂ | 1 | Q34 | CF₃ | CF₃ | — | — | |
| 6-79 | CH(CH₃)CH₂SO₂CH₃ (S)-isomer | H | H | C—I | CH | CH | CH | C—Cl | C—F | C—(A)r—Q | CH | CH | CH₂ | 1 | Q34 | CF₃ | CF₃ | — | — | |
| 6-80 | CH(CH₃)CH₂SCH₃ (S)-isomer | H | H | C—SCH₃ | CH | CH | CH | C—Cl | C—F | C—(A)r—Q | CH | CH | CH₂ | 1 | Q8 | CF₃ | H | CF₃ | — | |
| 6-81 | CH(CH₃)CH₂SCH₃ (S)-isomer | H | H | C—SCH₃ | CH | CH | CH | C—Cl | C—F | C—(A)r—Q | CH | CH | CH₂ | 1 | Q34 | CF₃ | CF₃ | — | — | |
| 6-82 | CH(CH₃)CH₂SCH₃ (S)-isomer | H | H | C—SO₂CH₃ | CH | CH | CH | C—Cl | C—F | C—(A)r—Q | CH | CH | CH₂ | 1 | Q8 | CF₃ | H | CF₃ | — | |
| 6-83 | CH(CH₃)CH₂SOCH₃ (S)-isomer | H | H | C—SO₂CH₃ | CH | CH | CH | C—Cl | C—F | C—(A)r—Q | CH | CH | CH₂ | 1 | Q8 | CF₃ | H | CF₃ | — | |
| 6-84 | CH(CH₃)CH₂SO₂CH₃ (S)-isomer | H | H | C—SO₂CH₃ | CH | CH | CH | C—Cl | C—F | C—(A)r—Q | CH | CH | CH₂ | 1 | Q8 | CF₃ | H | CF₃ | — | |
| 6-85 | CH(CH₃)CH₂SCH₃ (S)-isomer | H | H | C—SO₂CH₃ | CH | CH | CH | C—Cl | C—F | C—(A)r—Q | CH | CH | CH₂ | 1 | Q34 | CF₃ | CF₃ | — | — | |
| 6-86 | CH(CH₃)CH₂SOCH₃ (S)-isomer | H | H | C—SO₂CH₃ | CH | CH | CH | C—Cl | C—F | C—(A)r—Q | CH | CH | CH₂ | 1 | Q34 | CF₃ | CF₃ | — | — | |
| 6-87 | CH(CH₃)CH₂SO₂CH₃ (S)-isomer | H | H | C—SO₂CH₃ | CH | CH | CH | C—Cl | C—F | C—(A)r—Q | CH | CH | CH₂ | 1 | Q34 | CF₃ | CF₃ | — | — | |
| 6-88 | CH(CH₃)CH₂SCH₃ (S)-isomer | H | H | C—Cl | CH | CH | CH | C—Cl | C—F | C—(A)r—Q | CH | CH | CH₂ | 1 | Q34 | CF₂CHF₂ | CF₃ | — | — | |
| 6-89 | CH(CH₃)CH₂SCH₃ (S)-isomer | H | H | C—Cl | CH | CH | CH | C—Cl | C—F | C—(A)r—Q | CH | CH | CH₂ | 1 | Q34 | CF₃ | CF₂CHF₂ | — | — | |
| 6-90 | CH(CH₃)CH₂SCH₃ (S)-isomer | H | H | C—Br | CH | CH | CH | C—Cl | C—F | C—(A)r—Q | CH | CH | CH₂ | 1 | Q34 | CF₂CHF₂ | CF₃ | — | — | |
| 6-91 | CH(CH₃)CH₂SCH₃ (S)-isomer | H | H | C—Br | CH | CH | CH | C—Cl | C—F | C—(A)r—Q | CH | CH | CH₂ | 1 | Q34 | CF₃ | CF₂CHF₂ | — | — | |
| 6-92 | CH(CH₃)CH₂SCH₃ (S)-isomer | H | H | C—I | CH | CH | CH | C—Cl | C—F | C—(A)r—Q | CH | CH | CH₂ | 1 | Q34 | CF₂CHF₂ | CF₃ | — | — | |
| 6-93 | CH(CH₃)CH₂SCH₃ (S)-isomer | H | H | C—I | CH | CH | CH | C—Cl | C—F | C—(A)r—Q | CH | CH | CH₂ | 1 | Q34 | CF₃ | CF₂CHF₂ | — | — | |
| 6-94 | CH(CH₃)CH₂SCH₃ (S)-isomer | H | H | C—SCH₃ | CH | CH | CH | C—Cl | C—F | C—(A)r—Q | CH | CH | CH₂ | 1 | Q34 | CF₂CHF₂ | CF₃ | — | — | |
| 6-95 | CH(CH₃)CH₂SCH₃ (S)-isomer | H | H | C—SCH₃ | CH | CH | CH | C—Cl | C—F | C—(A)r—Q | CH | CH | CH₂ | 1 | Q34 | CF₃ | CF₂CHF₂ | — | — | |
| 6-96 | CH(CH₃)CH₂SCH₃ (S)-isomer | H | H | C—SO₂CH₃ | CH | CH | CH | C—Cl | C—F | C—(A)r—Q | CH | CH | CH₂ | 1 | Q34 | CF₂CHF₂ | CF₃ | — | — | |
| 6-97 | CH(CH₃)CH₂SCH₃ (S)-isomer | H | H | C—SO₂CH₃ | CH | CH | CH | C—Cl | C—F | C—(A)r—Q | CH | CH | CH₂ | 1 | Q34 | CF₃ | CF₂CHF₂ | — | — | |
| 6-98 | CH(CH₃)CH₂SCH₃ (S)-isomer | H | H | C—Cl | CH | CH | CH | C—Cl | C—F | C—(A)r—Q | CH | CH | CH₂ | 1 | Q34 | C₂F₅ | CF₃ | — | — | |
| 6-99 | CH(CH₃)CH₂SCH₃ (S)-isomer | H | H | C—Br | CH | CH | CH | C—Cl | C—F | C—(A)r—Q | CH | CH | CH₂ | 1 | Q34 | C₂F₅ | CF₃ | — | — | |
| 6-100 | CH(CH₃)CH₂SCH₃ (S)-isomer | H | H | C—I | CH | CH | CH | C—Cl | C—F | C—(A)r—Q | CH | CH | CH₂ | 1 | Q34 | C₂F₅ | CF₃ | — | — | |
| 6-101 | CH(CH₃)CH₂SCH₃ (S)-isomer | H | H | C—SCH₃ | CH | CH | CH | C—Cl | C—F | C—(A)r—Q | CH | CH | CH₂ | 1 | Q34 | C₂F₅ | CF₃ | — | — | |
| 6-102 | CH(CH₃)CH₂SCH₃ (S)-isomer | H | H | C—SO₂CH₃ | CH | CH | CH | C—Cl | C—F | C—(A)r—Q | CH | CH | CH₂ | 1 | Q34 | C₂F₅ | CF₃ | — | — | |
| 6-103 | CH(CH₃)CH₂SCH₃ (S)-isomer | H | H | C—Cl | CH | CH | CH | C—CH₃ | C—Cl | C—(A)r—Q | CH | CH | CH₂ | 1 | Q8 | CF₃ | H | CF₃ | — | |
| 6-104 | CH(CH₃)CH₂SCH₃ (S)-isomer | H | H | C—Br | CH | CH | CH | C—CH₃ | C—Cl | C—(A)r—Q | CH | CH | CH₂ | 1 | Q8 | CF₃ | H | CF₃ | — | |
| 6-105 | CH(CH₃)CH₂SCH₃ (S)-isomer | H | H | C—I | CH | CH | CH | C—CH₃ | C—Cl | C—(A)r—Q | CH | CH | CH₂ | 1 | Q8 | CF₃ | H | CF₃ | — | |
| 6-106 | CH(CH₃)CH₂SCH₃ (S)-isomer | H | H | C—SCH₃ | CH | CH | CH | C—CH₃ | C—Cl | C—(A)r—Q | CH | CH | CH₂ | 1 | Q8 | CF₃ | H | CF₃ | — | |
| 6-107 | CH(CH₃)CH₂SCH₃ (S)-isomer | H | H | C—SO₂CH₃ | CH | CH | CH | C—CH₃ | C—Cl | C—(A)r—Q | CH | CH | CH₂ | 1 | Q8 | CF₃ | H | CF₃ | — | |
| 6-108 | CH(CH₃)CH₂SCH₃ (S)-isomer | H | H | C—Cl | CH | CH | CH | C—CH₃ | C—Cl | C—(A)r—Q | CH | CH | CH₂ | 1 | Q34 | CF₃ | CF₃ | — | — | |
| 6-109 | CH(CH₃)CH₂SCH₃ (S)-isomer | H | H | C—Br | CH | CH | CH | C—CH₃ | C—Cl | C—(A)r—Q | CH | CH | CH₂ | 1 | Q34 | CF₃ | CF₃ | — | — | |
| 6-110 | CH(CH₃)CH₂SCH₃ (S)-isomer | H | H | C—I | CH | CH | CH | C—CH₃ | C—Cl | C—(A)r—Q | CH | CH | CH₂ | 1 | Q34 | CF₃ | CF₃ | — | — | |
| 6-111 | CH(CH₃)CH₂SCH₃ (S)-isomer | H | H | C—SCH₃ | CH | CH | CH | C—CH₃ | C—Cl | C—(A)r—Q | CH | CH | CH₂ | 1 | Q34 | CF₃ | CF₃ | — | — | |
| 6-112 | CH(CH₃)CH₂SCH₃ (S)-isomer | H | H | C—SO₂CH₃ | CH | CH | CH | C—CH₃ | C—Cl | C—(A)r—Q | CH | CH | CH₂ | 1 | Q34 | CF₃ | CF₃ | — | — | |
| 6-113 | CH(CH₃)CH₂SCH₃ (S)-isomer | H | H | C—Cl | CH | CH | CH | C—Cl | C—Cl | C—(A)r—Q | CH | CH | CH₂ | 1 | Q8 | CF₃ | H | CF₃ | — | |
| 6-114 | CH(CH₃)CH₂SCH₃ (S)-isomer | H | H | C—Br | CH | CH | CH | C—Cl | C—Cl | C—(A)r—Q | CH | CH | CH₂ | 1 | Q8 | CF₃ | H | CF₃ | — | |
| 6-115 | CH(CH₃)CH₂SCH₃ (S)-isomer | H | H | C—I | CH | CH | CH | C—Cl | C—Cl | C—(A)r—Q | CH | CH | CH₂ | 1 | Q8 | CF₃ | H | CF₃ | — | |
| 6-116 | CH(CH₃)CH₂SCH₃ (S)-isomer | H | H | C—SCH₃ | CH | CH | CH | C—Cl | C—Cl | C—(A)r—Q | CH | CH | CH₂ | 1 | Q8 | CF₃ | H | CF₃ | — | |
| 6-117 | CH(CH₃)CH₂SCH₃ (S)-isomer | H | H | C—SO₂CH₃ | CH | CH | CH | C—Cl | C—Cl | C—(A)r—Q | CH | CH | CH₂ | 1 | Q8 | CF₃ | H | CF₃ | — | |
| 6-118 | CH(CH₃)CH₂SCH₃ (S)-isomer | H | H | C—Cl | CH | CH | CH | C—Cl | C—Cl | C—(A)r—Q | CH | CH | CH₂ | 1 | Q34 | CF₃ | CF₃ | — | — | |
| 6-119 | CH(CH₃)CH₂SCH₃ (S)-isomer | H | H | C—Br | CH | CH | CH | C—Cl | C—Cl | C—(A)r—Q | CH | CH | CH₂ | 1 | Q34 | CF₃ | CF₃ | — | — | |
| 6-120 | CH(CH₃)CH₂SCH₃ (S)-isomer | H | H | C—I | CH | CH | CH | C—Cl | C—Cl | C—(A)r—Q | CH | CH | CH₂ | 1 | Q34 | CF₃ | CF₃ | — | — | |
| 6-121 | CH(CH₃)CH₂SCH₃ (S)-isomer | H | H | C—SCH₃ | CH | CH | CH | C—Cl | C—Cl | C—(A)r—Q | CH | CH | CH₂ | 1 | Q34 | CF₃ | CF₃ | — | — | |
| 6-122 | CH(CH₃)CH₂SCH₃ (S)-isomer | H | H | C—SO₂CH₃ | CH | CH | CH | C—Cl | C—Cl | C—(A)r—Q | CH | CH | CH₂ | 1 | Q34 | CF₃ | CF₃ | — | — | |
| 6-123 | CH(CH₃)CH₂SCH₃ (S)-isomer | H | H | C—Cl | CH | CH | CH | C—CH₃ | C—CF₃ | C—(A)r—Q | CH | CH | CH₂ | 1 | Q8 | CF₃ | H | H | — | |
| 6-124 | CH(CH₃)CH₂SCH₃ (S)-isomer | H | H | C—SO₂CH₃ | CH | CH | CH | C—CH₃ | C—CF₃ | C—(A)r—Q | CH | CH | CH₂ | 1 | Q34 | CF₃ | H | — | — | |
| 6-125 | CH(CH₃)CH₂SCH₃ (S)-isomer | H | H | C—Cl | CH | CH | CH | C—CH₃ | C—CF₃ | C—(A)r—Q | CH | CH | CH₂ | 1 | Q8 | CF₃ | H | CF₃ | — | |
| 6-126 | CH(CH₃)CH₂SCH₃ (S)-isomer | H | H | C—Br | CH | CH | CH | C—CH₃ | C—CF₃ | C—(A)r—Q | CH | CH | CH₂ | 1 | Q8 | CF₃ | H | CF₃ | — | |
| 6-127 | CH(CH₃)CH₂SCH₃ (S)-isomer | H | H | C—I | CH | CH | CH | C—CH₃ | C—CF₃ | C—(A)r—Q | CH | CH | CH₂ | 1 | Q8 | CF₃ | H | CF₃ | — | |
| 6-128 | CH(CH₃)CH₂SCH₃ (S)-isomer | H | H | C—SCH₃ | CH | CH | CH | C—CH₃ | C—CF₃ | C—(A)r—Q | CH | CH | CH₂ | 1 | Q8 | CF₃ | H | CF₃ | — | |
| 6-129 | CH(CH₃)CH₂SCH₃ (S)-isomer | H | H | C—SO₂CH₃ | CH | CH | CH | C—CH₃ | C—CF₃ | C—(A)r—Q | CH | CH | CH₂ | 1 | Q8 | CF₃ | H | CF₃ | — | |
| 6-130 | CH(CH₃)CH₂SCH₃ (S)-isomer | H | H | C—Cl | CH | CH | CH | C—CH₃ | C—CF₃ | C—(A)r—Q | CH | CH | CH₂ | 1 | Q34 | CF₃ | CF₃ | — | — | |
| 6-131 | CH(CH₃)CH₂SCH₃ (S)-isomer | H | H | C—Br | CH | CH | CH | C—CH₃ | C—CF₃ | C—(A)r—Q | CH | CH | CH₂ | 1 | Q34 | CF₃ | CF₃ | — | — | |
| 6-132 | CH(CH₃)CH₂SCH₃ (S)-isomer | H | H | C—I | CH | CH | CH | C—CH₃ | C—CF₃ | C—(A)r—Q | CH | CH | CH₂ | 1 | Q34 | CF₃ | CF₃ | — | — | |
| 6-133 | CH(CH₃)CH₂SCH₃ (S)-isomer | H | H | C—SCH₃ | CH | CH | CH | C—CH₃ | C—CF₃ | C—(A)r—Q | CH | CH | CH₂ | 1 | Q34 | CF₃ | CF₃ | — | — | |
| 6-134 | CH(CH₃)CH₂SCH₃ (S)-isomer | H | H | C—SO₂CH₃ | CH | CH | CH | C—CH₃ | C—CF₃ | C—(A)r—Q | CH | CH | CH₂ | 1 | Q34 | CF₃ | CF₃ | — | — | |
| 6-135 | CH(CH₃)CH₂SCH₃ (S)-isomer | H | H | C—Cl | CH | CH | CH | C—Cl | C—CF₃ | C—(A)r—Q | CH | CH | CH₂ | 1 | Q8 | CF₃ | H | H | — | |
| 6-136 | CH(CH₃)CH₂SCH₃ (S)-isomer | H | H | C—SCH₃ | CH | CH | CH | C—Cl | C—CF₃ | C—(A)r—Q | CH | CH | CH₂ | 1 | Q8 | CF₃ | H | H | — | |
| 6-137 | CH(CH₃)CH₂SCH₃ (S)-isomer | H | H | C—Cl | CH | CH | CH | C—Cl | C—CF₃ | C—(A)r—Q | CH | CH | CH₂ | 1 | Q34 | CF₃ | H | — | — | |
| 6-138 | CH(CH₃)CH₂SCH₃ (S)-isomer | H | H | C—Cl | CH | CH | CH | C—Cl | C—CF₃ | C—(A)r—Q | CH | CH | CH₂ | 1 | Q8 | CF₃ | H | CF₃ | — | |
| 6-139 | CH(CH₃)CH₂SCH₃ (S)-isomer | H | H | C—Br | CH | CH | CH | C—Cl | C—CF₃ | C—(A)r—Q | CH | CH | CH₂ | 1 | Q8 | CF₃ | H | CF₃ | — | |
| 6-140 | CH(CH₃)CH₂SCH₃ (S)-isomer | H | H | C—I | CH | CH | CH | C—Cl | C—CF₃ | C—(A)r—Q | CH | CH | CH₂ | 1 | Q8 | CF₃ | H | CF₃ | — | |
| 6-141 | CH(CH₃)CH₂SCH₃ (S)-isomer | H | H | C—SCH₃ | CH | CH | CH | C—Cl | C—CF₃ | C—(A)r—Q | CH | CH | CH₂ | 1 | Q8 | CF₃ | H | CF₃ | — | |
| 6-142 | CH(CH₃)CH₂SCH₃ (S)-isomer | H | H | C—SO₂CH₃ | CH | CH | CH | C—Cl | C—CF₃ | C—(A)r—Q | CH | CH | CH₂ | 1 | Q8 | CF₃ | H | CF₃ | — | |
| 6-143 | CH(CH₃)CH₂SCH₃ (S)-isomer | H | H | C—Cl | CH | CH | CH | C—Cl | C—CF₃ | C—(A)r—Q | CH | CH | CH₂ | 1 | Q34 | CF₃ | CF₃ | — | — | |
| 6-144 | CH(CH₃)CH₂SCH₃ (S)-isomer | H | H | C—Br | CH | CH | CH | C—Cl | C—CF₃ | C—(A)r—Q | CH | CH | CH₂ | 1 | Q34 | CF₃ | CF₃ | — | — | |
| 6-145 | CH(CH₃)CH₂SCH₃ (S)-isomer | H | H | C—I | CH | CH | CH | C—Cl | C—CF₃ | C—(A)r—Q | CH | CH | CH₂ | 1 | Q34 | CF₃ | CF₃ | — | — | |
| 6-146 | CH(CH₃)CH₂SCH₃ (S)-isomer | H | H | C—SCH₃ | CH | CH | CH | C—Cl | C—CF₃ | C—(A)r—Q | CH | CH | CH₂ | 1 | Q34 | CF₃ | CF₃ | — | — | |
| 6-147 | CH(CH₃)CH₂SCH₃ (S)-isomer | H | H | C—SO₂CH₃ | CH | CH | CH | C—Cl | C—CF₃ | C—(A)r—Q | CH | CH | CH₂ | 1 | Q34 | CF₃ | CF₃ | — | — | |
| 6-148 | CH(CH₃)CH₂SCH₃ (S)-isomer | H | H | C—Cl | CH | CH | CH | C—CH₃ | C—F | C—(A)r—Q | CH | CH | CH(CH₃) | 1 | Q8 | CF₃ | H | CF₃ | — | |
| 6-149 | CH(CH₃)CH₂SCH₃ (S)-isomer | H | H | C—Br | CH | CH | CH | C—CH₃ | C—F | C—(A)r—Q | CH | CH | CH(CH₃) | 1 | Q8 | CF₃ | H | CF₃ | — | |
| 6-150 | CH(CH₃)CH₂SCH₃ (S)-isomer | H | H | C—I | CH | CH | CH | C—CH₃ | C—F | C—(A)r—Q | CH | CH | CH(CH₃) | 1 | Q8 | CF₃ | H | CF₃ | — | |
| 6-151 | CH(CH₃)CH₂SCH₃ (S)-isomer | H | H | C—SCH₃ | CH | CH | CH | C—CH₃ | C—F | C—(A)r—Q | CH | CH | CH(CH₃) | 1 | Q8 | CF₃ | H | CF₃ | — | |
| 6-152 | CH(CH₃)CH₂SCH₃ (S)-isomer | H | H | C—SO₂CH₃ | CH | CH | CH | C—CH₃ | C—F | C—(A)r—Q | CH | CH | CH(CH₃) | 1 | Q8 | CF₃ | H | CF₃ | — | |
| 6-153 | CH(CH₃)CH₂SCH₃ (S)-isomer | H | H | C—Cl | CH | CH | CH | C—CH₃ | C—F | C—(A)r—Q | CH | CH | CH(CH₃) | 1 | Q34 | CF₃ | CF₃ | — | — | |
| 6-154 | CH(CH₃)CH₂SCH₃ (S)-isomer | H | H | C—Br | CH | CH | CH | C—CH₃ | C—F | C—(A)r—Q | CH | CH | CH(CH₃) | 1 | Q34 | CF₃ | CF₃ | — | — | |
| 6-155 | CH(CH₃)CH₂SCH₃ (S)-isomer | H | H | C—I | CH | CH | CH | C—CH₃ | C—F | C—(A)r—Q | CH | CH | CH(CH₃) | 1 | Q34 | CF₃ | CF₃ | — | — | |
| 6-156 | CH(CH₃)CH₂SCH₃ (S)-isomer | H | H | C—SCH₃ | CH | CH | CH | C—CH₃ | C—F | C—(A)r—Q | CH | CH | CH(CH₃) | 1 | Q34 | CF₃ | CF₃ | — | — | |
| 6-157 | CH(CH₃)CH₂SCH₃ (S)-isomer | H | H | C—SO₂CH₃ | CH | CH | CH | C—CH₃ | C—F | C—(A)r—Q | CH | CH | CH(CH₃) | 1 | Q34 | CF₃ | CF₃ | — | — | |
| 6-158 | CH(CH₃)CH₂SCH₃ (S)-isomer | H | H | C—Cl | CH | CH | CH | C—Cl | C—F | C—(A)r—Q | CH | CH | CH(CH₃) | 1 | Q8 | CF₃ | H | CF₃ | — | |
| 6-159 | CH(CH₃)CH₂SCH₃ (S)-isomer | H | H | C—Br | CH | CH | CH | C—Cl | C—F | C—(A)r—Q | CH | CH | CH(CH₃) | 1 | Q8 | CF₃ | H | CF₃ | — | |
| 6-160 | CH(CH₃)CH₂SCH₃ (S)-isomer | H | H | C—I | CH | CH | CH | C—Cl | C—F | C—(A)r—Q | CH | CH | CH(CH₃) | 1 | Q8 | CF₃ | H | CF₃ | — | |
| 6-161 | CH(CH₃)CH₂SCH₃ (S)-isomer | H | H | C—SCH₃ | CH | CH | CH | C—Cl | C—F | C—(A)r—Q | CH | CH | CH(CH₃) | 1 | Q8 | CF₃ | H | CF₃ | — | |
| 6-162 | CH(CH₃)CH₂SCH₃ (S)-isomer | H | H | C—SO₂CH₃ | CH | CH | CH | C—Cl | C—F | C—(A)r—Q | CH | CH | CH(CH₃) | 1 | Q8 | CF₃ | H | CF₃ | — | |
| 6-163 | CH(CH₃)CH₂SCH₃ (S)-isomer | H | H | C—Cl | CH | CH | CH | C—Cl | C—F | C—(A)r—Q | CH | CH | CH(CH₃) | 1 | Q34 | CF₃ | CF₃ | — | — | |
| 6-164 | CH(CH₃)CH₂SCH₃ (S)-isomer | H | H | C—Br | CH | CH | CH | C—Cl | C—F | C—(A)r—Q | CH | CH | CH(CH₃) | 1 | Q34 | CF₃ | CF₃ | — | — | |
| 6-165 | CH(CH₃)CH₂SCH₃ (S)-isomer | H | H | C—I | CH | CH | CH | C—Cl | C—F | C—(A)r—Q | CH | CH | CH(CH₃) | 1 | Q34 | CF₃ | CF₃ | — | — | |
| 6-166 | CH(CH₃)CH₂SCH₃ (S)-isomer | H | H | C—SCH₃ | CH | CH | CH | C—Cl | C—F | C—(A)r—Q | CH | CH | CH(CH₃) | 1 | Q34 | CF₃ | CF₃ | — | — | |
| 6-167 | CH(CH₃)CH₂SCH₃ (S)-isomer | H | H | C—SO₂CH₃ | CH | CH | CH | C—Cl | C—F | C—(A)r—Q | CH | CH | CH(CH₃) | 1 | Q34 | CF₃ | CF₃ | — | — | |
| 6-168 | CH(CH₃)CH₂SCH₃ (S)-isomer | H | H | C—Cl | CH | CH | CH | C—Cl | C—Cl | C—(A)r—Q | CH | CH | CH(CH₃) | 1 | Q8 | CF₃ | H | H | — | |
| 6-169 | CH(CH₃)CH₂SCH₃ (S)-isomer | H | H | C—Cl | CH | CH | CH | C—Cl | C—Cl | C—(A)r—Q | CH | CH | CH(CH₃) | 1 | Q34 | CF₃ | H | — | — | |
| 6-170 | CH(CH₃)CH₂SCH₃ (S)-isomer | H | H | C—Cl | CH | CH | CH | C—Cl | C—Cl | C—(A)r—Q | CH | CH | CH(CH₃) | 1 | Q8 | CF₃ | H | CF₃ | — | |
| 6-171 | CH(CH₃)CH₂SCH₃ (S)-isomer | H | H | C—Br | CH | CH | CH | C—Cl | C—Cl | C—(A)r—Q | CH | CH | CH(CH₃) | 1 | Q8 | CF₃ | H | CF₃ | — | |
| 6-172 | CH(CH₃)CH₂SCH₃ (S)-isomer | H | H | C—I | CH | CH | CH | C—Cl | C—Cl | C—(A)r—Q | CH | CH | CH(CH₃) | 1 | Q8 | CF₃ | H | CF₃ | — | |
| 6-173 | CH(CH₃)CH₂SCH₃ (S)-isomer | H | H | C—SCH₃ | CH | CH | CH | C—Cl | C—Cl | C—(A)r—Q | CH | CH | CH(CH₃) | 1 | Q8 | CF₃ | H | CF₃ | — | |
| 6-174 | CH(CH₃)CH₂SCH₃ (S)-isomer | H | H | C—SO₂CH₃ | CH | CH | CH | C—Cl | C—Cl | C—(A)r—Q | CH | CH | CH(CH₃) | 1 | Q8 | CF₃ | H | CF₃ | — | |
| 6-175 | CH(CH₃)CH₂SCH₃ (S)-isomer | H | H | C—Cl | CH | CH | CH | C—Cl | C—Cl | C—(A)r—Q | CH | CH | CH(CH₃) | 1 | Q34 | CF₃ | CF₃ | — | — | |
| 6-176 | CH(CH₃)CH₂SCH₃ (S)-isomer | H | H | C—Br | CH | CH | CH | C—Cl | C—Cl | C—(A)r—Q | CH | CH | CH(CH₃) | 1 | Q34 | CF₃ | CF₃ | — | — | |
| 6-177 | CH(CH₃)CH₂SCH₃ (S)-isomer | H | H | C—I | CH | CH | CH | C—Cl | C—Cl | C—(A)r—Q | CH | CH | CH(CH₃) | 1 | Q34 | CF₃ | CF₃ | — | — | |
| 6-178 | CH(CH₃)CH₂SCH₃ (S)-isomer | H | H | C—SCH₃ | CH | CH | CH | C—Cl | C—Cl | C—(A)r—Q | CH | CH | CH(CH₃) | 1 | Q34 | CF₃ | CF₃ | — | — | |

TABLE 7

| No. | R¹ | R² | R³ | W¹ | W² | W³ | W⁴ | W⁵ | W⁶ | W⁷ | W⁸ | W⁹ | A | r | Q | R⁴ | R⁵ | R⁶ | R⁷ | Physical property |
|---|---|---|---|---|---|---|---|---|---|---|---|---|---|---|---|---|---|---|---|---|
| 7-1 | CH(CH₃)CH₂SCH₃ (S)-isomer | H | H | C—Br | CH | CH | CH | C—CH₃ | CH | C—(A)r—Q | C—F | CH | CH₂ | 1 | Q8 | CF₃ | H | H | — | |
| 7-2 | CH(CH₃)CH₂SCH₃ (S)-isomer | H | H | C—Br | CH | CH | CH | C—CH₃ | CH | C—(A)r—Q | C—F | CH | CH₂ | 1 | Q34 | CF₃ | H | — | — | |
| 7-3 | CH(CH₃)CH₂SCH₃ (S)-isomer | H | H | C—Br | CH | CH | CH | C—CH₃ | CH | C—(A)r—Q | C—F | CH | CH₂ | 1 | Q34 | C₂F₅ | H | — | — | |
| 7-4 | CH(CH₃)CH₂SCH₃ (S)-isomer | H | H | C—Cl | CH | CH | CH | C—CH₃ | CH | C—(A)r—Q | C—F | CH | CH₂ | 1 | Q8 | CF₃ | H | CF₃ | — | NMR |
| 7-5 | CH(CH₃)CH₂SOCH₃ (S)-isomer | H | H | C—Cl | CH | CH | CH | C—CH₃ | CH | C—(A)r—Q | C—F | CH | CH₂ | 1 | Q8 | CF₃ | H | CF₃ | — | NMR |
| 7-6 | CH(CH₃)CH₂SO₂CH₃ (S)-isomer | H | H | C—Cl | CH | CH | CH | C—CH₃ | CH | C—(A)r—Q | C—F | CH | CH₂ | 1 | Q8 | CF₃ | H | CF₃ | — | NMR |
| 7-7 | CH(CH₃)CH₂SCH₃ (S)-isomer | H | H | C—Cl | CH | CH | CH | C—CH₃ | CH | C—(A)r—Q | C—F | CH | CH₂ | 1 | Q34 | CF₃ | CF₃ | — | — | NMR |
| 7-8 | CH(CH₃)CH₂SOCH₃ (S)-isomer | H | H | C—Cl | CH | CH | CH | C—CH₃ | CH | C—(A)r—Q | C—F | CH | CH₂ | 1 | Q34 | CF₃ | CF₃ | — | — | logP (acid) 2.63 |
| 7-9 | CH(CH₃)CH₂SO₂CH₃ (S)-isomer | H | H | C—Cl | CH | CH | CH | C—CH₃ | CH | C—(A)r—Q | C—F | CH | CH₂ | 1 | Q34 | CF₃ | CF₃ | — | — | logP (acid) 3.02 |
| 7-10 | CH(CH₃)CH₂SCH₃ (S)-isomer | H | H | C—Br | CH | CH | CH | C—CH₃ | CH | C—(A)r—Q | C—F | CH | CH₂ | 1 | Q8 | CF₃ | H | CF₃ | — | logP (acid) 4.37 |
| 7-11 | CH(CH₃)CH₂SOCH₃ (S)-isomer | H | H | C—Br | CH | CH | CH | C—CH₃ | CH | C—(A)r—Q | C—F | CH | CH₂ | 1 | Q8 | CF₃ | H | CF₃ | — | NMR |
| 7-12 | CH(CH₃)CH₂SO₂CH₃ (S)-isomer | H | H | C—Br | CH | CH | CH | C—CH₃ | CH | C—(A)r—Q | C—F | CH | CH₂ | 1 | Q8 | CF₃ | H | CF₃ | — | NMR |
| 7-13 | CH(CH₃)CH₂SCH₃ (S)-isomer | H | H | C—Br | CH | CH | CH | C—CH₃ | CH | C—(A)r—Q | C—F | CH | CH₂ | 1 | Q34 | CF₃ | CF₃ | — | — | logP (acid) 4.07 |
| 7-14 | CH(CH₃)CH₂SOCH₃ (S)-isomer | H | H | C—Br | CH | CH | CH | C—CH₃ | CH | C—(A)r—Q | C—F | CH | CH₂ | 1 | Q34 | CF₃ | CF₃ | — | — | logP (acid) 2.68 |
| 7-15 | CH(CH₃)CH₂SO₂CH₃ (S)-isomer | H | H | C—Br | CH | CH | CH | C—CH₃ | CH | C—(A)r—Q | C—F | CH | CH₂ | 1 | Q34 | CF₃ | CF₃ | — | — | logP (acid) 3.08 |
| 7-16 | CH(CH₃)CH₂SCH₃ (S)-isomer | H | H | C—I | CH | CH | CH | C—CH₃ | CH | C—(A)r—Q | C—F | CH | CH₂ | 1 | Q8 | CF₃ | H | CF₃ | — | NMR |
| 7-17 | CH(CH₃)CH₂SOCH₃ (S)-isomer | H | H | C—I | CH | CH | CH | C—CH₃ | CH | C—(A)r—Q | C—F | CH | CH₂ | 1 | Q8 | CF₃ | H | CF₃ | — | NMR |
| 7-18 | CH(CH₃)CH₂SO₂CH₃ (S)-isomer | H | H | C—I | CH | CH | CH | C—CH₃ | CH | C—(A)r—Q | C—F | CH | CH₂ | 1 | Q8 | CF₃ | H | CF₃ | — | NMR |
| 7-19 | CH(CH₃)CH₂SCH₃ (S)-isomer | H | H | C—I | CH | CH | CH | C—CH₃ | CH | C—(A)r—Q | C—F | CH | CH₂ | 1 | Q34 | CF₃ | CF₃ | — | — | |
| 7-20 | CH(CH₃)CH₂SOCH₃ (S)-isomer | H | H | C—I | CH | CH | CH | C—CH₃ | CH | C—(A)r—Q | C—F | CH | CH₂ | 1 | Q34 | CF₃ | CF₃ | — | — | |
| 7-21 | CH(CH₃)CH₂SO₂CH₃ (S)-isomer | H | H | C—I | CH | CH | CH | C—CH₃ | CH | C—(A)r—Q | C—F | CH | CH₂ | 1 | Q34 | CF₃ | CF₃ | — | — | |
| 7-22 | CH(CH₃)CH₂SCH₃ (S)-isomer | H | H | C—SCH₃ | CH | CH | CH | C—CH₃ | CH | C—(A)r—Q | C—F | CH | CH₂ | 1 | Q8 | CF₃ | H | CF₃ | — | NMR |
| 7-23 | CH(CH₃)CH₂SCH₃ (S)-isomer | H | H | C—SCH₃ | CH | CH | CH | C—CH₃ | CH | C—(A)r—Q | C—F | CH | CH₂ | 1 | Q34 | CF₃ | CF₃ | — | — | logP (acid) 3.96 |
| 7-24 | CH(CH₃)CH₂SCH₃ (S)-isomer | H | H | C—SO₂CH₃ | CH | CH | CH | C—CH₃ | CH | C—(A)r—Q | C—F | CH | CH₂ | 1 | Q8 | CF₃ | H | CF₃ | — | |
| 7-25 | CH(CH₃)CH₂SOCH₃ (S)-isomer | H | H | C—SO₂CH₃ | CH | CH | CH | C—CH₃ | CH | C—(A)r—Q | C—F | CH | CH₂ | 1 | Q8 | CF₃ | H | CF₃ | — | |
| 7-26 | CH(CH₃)CH₂SO₂CH₃ (S)-isomer | H | H | C—SO₂CH₃ | CH | CH | CH | C—CH₃ | CH | C—(A)r—Q | C—F | CH | CH₂ | 1 | Q8 | CF₃ | H | CF₃ | — | |
| 7-27 | CH(CH₃)CH₂SCH₃ (S)-isomer | H | H | C—SO₂CH₃ | CH | CH | CH | C—CH₃ | CH | C—(A)r—Q | C—F | CH | CH₂ | 1 | Q34 | CF₃ | CF₃ | — | — | |
| 7-28 | CH(CH₃)CH₂SOCH₃ (S)-isomer | H | H | C—SO₂CH₃ | CH | CH | CH | C—CH₃ | CH | C—(A)r—Q | C—F | CH | CH₂ | 1 | Q34 | CF₃ | CF₃ | — | — | |
| 7-29 | CH(CH₃)CH₂SO₂CH₃ (S)-isomer | H | H | C—SO₂CH₃ | CH | CH | CH | C—CH₃ | CH | C—(A)r—Q | C—F | CH | CH₂ | 1 | Q34 | CF₃ | CF₃ | — | — | |
| 7-30 | CH(CH₃)CH₂SCH₃ (S)-isomer | H | H | C—Cl | CH | CH | CH | C—CH₃ | CH | C—(A)r—Q | C—F | CH | CH₂ | 1 | Q34 | CF₂CHF₂ | CF₃ | — | — | |
| 7-31 | CH(CH₃)CH₂SCH₃ (S)-isomer | H | H | C—Cl | CH | CH | CH | C—CH₃ | CH | C—(A)r—Q | C—F | CH | CH₂ | 1 | Q34 | C₂F₅ | CF₃ | — | — | |
| 7-32 | CH(CH₃)CH₂SCH₃ (S)-isomer | H | H | C—Cl | CH | CH | CH | C—CH₃ | CH | C—(A)r—Q | C—F | CH | CH₂ | 1 | Q34 | CF₃ | CF₂CHF₂ | — | — | |
| 7-33 | CH(CH₃)CH₂SCH₃ (S)-isomer | H | H | C—I | CH | CH | CH | C—CH₃ | CH | C—(A)r—Q | C—F | CH | CH₂ | 1 | Q34 | CF₂CHF₂ | CF₃ | — | — | |
| 7-34 | CH(CH₃)CH₂SCH₃ (S)-isomer | H | H | C—I | CH | CH | CH | C—CH₃ | CH | C—(A)r—Q | C—F | CH | CH₂ | 1 | Q34 | C₂F₅ | CF₃ | — | — | |
| 7-35 | CH(CH₃)CH₂SCH₃ (S)-isomer | H | H | C—I | CH | CH | CH | C—CH₃ | CH | C—(A)r—Q | C—F | CH | CH₂ | 1 | Q34 | CF₃ | CF₂CHF₂ | — | — | |
| 7-36 | CH(CH₃)CH₂SCH₃ (S)-isomer | H | H | C—SCH₃ | CH | CH | CH | C—CH₃ | CH | C—(A)r—Q | C—F | CH | CH₂ | 1 | Q34 | CF₂CHF₂ | CF₃ | — | — | |
| 7-37 | CH(CH₃)CH₂SCH₃ (S)-isomer | H | H | C—SCH₃ | CH | CH | CH | C—CH₃ | CH | C—(A)r—Q | C—F | CH | CH₂ | 1 | Q34 | C₂F₅ | CF₃ | — | — | |
| 7-38 | CH(CH₃)CH₂SCH₃ (S)-isomer | H | H | C—SCH₃ | CH | CH | CH | C—CH₃ | CH | C—(A)r—Q | C—F | CH | CH₂ | 1 | Q34 | CF₃ | CF₂CHF₂ | — | — | |
| 7-39 | CH(CH₃)CH₂SCH₃ (S)-isomer | H | H | C—Br | CH | CH | CH | C—Cl | CH | C—(A)r—Q | C—F | CH | CH₂ | 1 | Q8 | CF₃ | H | H | — | |
| 7-40 | CH(CH₃)CH₂SCH₃ (S)-isomer | H | H | C—Br | CH | CH | CH | C—Cl | CH | C—(A)r—Q | C—F | CH | CH₂ | 1 | Q34 | CF₃ | H | — | — | |
| 7-41 | CH(CH₃)CH₂SCH₃ (S)-isomer | H | H | C—Cl | CH | CH | CH | C—Cl | CH | C—(A)r—Q | C—F | CH | CH₂ | 1 | Q8 | CF₃ | H | CF₃ | — | |
| 7-42 | CH(CH₃)CH₂SOCH₃ (S)-isomer | H | H | C—Cl | CH | CH | CH | C—Cl | CH | C—(A)r—Q | C—F | CH | CH₂ | 1 | Q8 | CF₃ | H | CF₃ | — | |
| 7-43 | CH(CH₃)CH₂SO₂CH₃ (S)-isomer | H | H | C—Cl | CH | CH | CH | C—Cl | CH | C—(A)r—Q | C—F | CH | CH₂ | 1 | Q8 | CF₃ | H | CF₃ | — | |
| 7-44 | CH(CH₃)CH₂SCH₃ (S)-isomer | H | H | C—Cl | CH | CH | CH | C—Cl | CH | C—(A)r—Q | C—F | CH | CH₂ | 1 | Q34 | CF₃ | CF₃ | — | — | NMR |
| 7-45 | CH(CH₃)CH₂SOCH₃ (S)-isomer | H | H | C—Cl | CH | CH | CH | C—Cl | CH | C—(A)r—Q | C—F | CH | CH₂ | 1 | Q34 | CF₃ | CF₃ | — | — | |
| 7-46 | CH(CH₃)CH₂SO₂CH₃ (S)-isomer | H | H | C—Cl | CH | CH | CH | C—Cl | CH | C—(A)r—Q | C—F | CH | CH₂ | 1 | Q34 | CF₃ | CF₃ | — | — | |
| 7-47 | CH(CH₃)CH₂SCH₃ (S)-isomer | H | H | C—Br | CH | CH | CH | C—Cl | CH | C—(A)r—Q | C—F | CH | CH₂ | 1 | Q8 | CF₃ | H | CF₃ | — | |
| 7-48 | CH(CH₃)CH₂SCH₃ (S)-isomer | H | H | C—Br | CH | CH | CH | C—Cl | CH | C—(A)r—Q | C—F | CH | CH₂ | 1 | Q34 | CF₃ | CF₃ | — | — | |
| 7-49 | CH(CH₃)CH₂SCH₃ (S)-isomer | H | H | C—I | CH | CH | CH | C—Cl | CH | C—(A)r—Q | C—F | CH | CH₂ | 1 | Q8 | CF₃ | H | CF₃ | — | |
| 7-50 | CH(CH₃)CH₂SOCH₃ (S)-isomer | H | H | C—I | CH | CH | CH | C—Cl | CH | C—(A)r—Q | C—F | CH | CH₂ | 1 | Q8 | CF₃ | H | CF₃ | — | |
| 7-51 | CH(CH₃)CH₂SO₂CH₃ (S)-isomer | H | H | C—I | CH | CH | CH | C—Cl | CH | C—(A)r—Q | C—F | CH | CH₂ | 1 | Q8 | CF₃ | H | CF₃ | — | |
| 7-52 | CH(CH₃)CH₂SCH₃ (S)-isomer | H | H | C—I | CH | CH | CH | C—Cl | CH | C—(A)r—Q | C—F | CH | CH₂ | 1 | Q34 | CF₃ | CF₃ | — | — | |
| 7-53 | CH(CH₃)CH₂SOCH₃ (S)-isomer | H | H | C—I | CH | CH | CH | C—Cl | CH | C—(A)r—Q | C—F | CH | CH₂ | 1 | Q34 | CF₃ | CF₃ | — | — | |
| 7-54 | CH(CH₃)CH₂SO₂CH₃ (S)-isomer | H | H | C—I | CH | CH | CH | C—Cl | CH | C—(A)r—Q | C—F | CH | CH₂ | 1 | Q34 | CF₃ | CF₃ | — | — | |
| 7-55 | CH(CH₃)CH₂SCH₃ (S)-isomer | H | H | C—SCH₃ | CH | CH | CH | C—Cl | CH | C—(A)r—Q | C—F | CH | CH₂ | 1 | Q8 | CF₃ | H | CF₃ | — | |
| 7-56 | CH(CH₃)CH₂SCH₃ (S)-isomer | H | H | C—SCH₃ | CH | CH | CH | C—Cl | CH | C—(A)r—Q | C—F | CH | CH₂ | 1 | Q34 | CF₃ | CF₃ | — | — | |
| 7-57 | CH(CH₃)CH₂SCH₃ (S)-isomer | H | H | C—SO₂CH₃ | CH | CH | CH | C—Cl | CH | C—(A)r—Q | C—F | CH | CH₂ | 1 | Q8 | CF₃ | H | CF₃ | — | |
| 7-58 | CH(CH₃)CH₂SCH₃ (S)-isomer | H | H | C—SO₂CH₃ | CH | CH | CH | C—Cl | CH | C—(A)r—Q | C—F | CH | CH₂ | 1 | Q34 | CF₃ | CF₃ | — | — | |
| 7-59 | CH(CH₃)CH₂SCH₃ (S)-isomer | H | H | C—Cl | CH | CH | CH | C—Cl | CH | C—(A)r—Q | C—F | CH | CH₂ | 1 | Q34 | CF₂CHF₂ | CF₃ | — | — | |
| 7-60 | CH(CH₃)CH₂SCH₃ (S)-isomer | H | H | C—Cl | CH | CH | CH | C—Cl | CH | C—(A)r—Q | C—F | CH | CH₂ | 1 | Q34 | C₂F₅ | CF₃ | — | — | |
| 7-61 | CH(CH₃)CH₂SCH₃ (S)-isomer | H | H | C—Cl | CH | CH | CH | C—Cl | CH | C—(A)r—Q | C—F | CH | CH₂ | 1 | Q34 | CF₃ | CF₂CHF₂ | — | — | |
| 7-62 | CH(CH₃)CH₂SCH₃ (S)-isomer | H | H | C—I | CH | CH | CH | C—Cl | CH | C—(A)r—Q | C—F | CH | CH₂ | 1 | Q34 | CF₂CHF₂ | CF₃ | — | — | |
| 7-63 | CH(CH₃)CH₂SCH₃ (S)-isomer | H | H | C—I | CH | CH | CH | C—Cl | CH | C—(A)r—Q | C—F | CH | CH₂ | 1 | Q34 | C₂F₅ | CF₃ | — | — | |
| 7-64 | CH(CH₃)CH₂SCH₃ (S)-isomer | H | H | C—I | CH | CH | CH | C—Cl | CH | C—(A)r—Q | C—F | CH | CH₂ | 1 | Q34 | CF₃ | CF₂CHF₂ | — | — | |
| 7-65 | CH(CH₃)CH₂SCH₃ (S)-isomer | H | H | C—SCH₃ | CH | CH | CH | C—Cl | CH | C—(A)r—Q | C—F | CH | CH₂ | 1 | Q34 | CF₂CHF₂ | CF₃ | — | — | |
| 7-66 | CH(CH₃)CH₂SCH₃ (S)-isomer | H | H | C—SCH₃ | CH | CH | CH | C—Cl | CH | C—(A)r—Q | C—F | CH | CH₂ | 1 | Q34 | C₂F₅ | CF₃ | — | — | |
| 7-67 | CH(CH₃)CH₂SCH₃ (S)-isomer | H | H | C—SCH₃ | CH | CH | CH | C—Cl | CH | C—(A)r—Q | C—F | CH | CH₂ | 1 | Q34 | CF₃ | CF₂CHF₂ | — | — | |
| 7-68 | CH(CH₃)CH₂SCH₃ (S)-isomer | H | H | C—Br | CH | CH | CH | C—CH₃ | CH | C—(A)r—Q | C—Cl | CH | CH₂ | 1 | Q8 | CF₃ | H | H | — | |
| 7-69 | CH(CH₃)CH₂SCH₃ (S)-isomer | H | H | C—Br | CH | CH | CH | C—CH₃ | CH | C—(A)r—Q | C—Cl | CH | CH₂ | 1 | Q34 | CF₃ | H | — | — | |
| 7-70 | CH(CH₃)CH₂SCH₃ (S)-isomer | H | H | C—Cl | CH | CH | CH | C—CH₃ | CH | C—(A)r—Q | C—Cl | CH | CH₂ | 1 | Q8 | CF₃ | H | CF₃ | — | |
| 7-71 | CH(CH₃)CH₂SCH₃ (S)-isomer | H | H | C—I | CH | CH | CH | C—CH₃ | CH | C—(A)r—Q | C—Cl | CH | CH₂ | 1 | Q8 | CF₃ | H | CF₃ | — | |
| 7-72 | CH(CH₃)CH₂SCH₃ (S)-isomer | H | H | C—SCH₃ | CH | CH | CH | C—CH₃ | CH | C—(A)r—Q | C—Cl | CH | CH₂ | 1 | Q8 | CF₃ | H | CF₃ | — | |
| 7-73 | CH(CH₃)CH₂SCH₃ (S)-isomer | H | H | C—Cl | CH | CH | CH | C—CH₃ | CH | C—(A)r—Q | C—Cl | CH | CH₂ | 1 | Q34 | CF₃ | CF₃ | — | — | |
| 7-74 | CH(CH₃)CH₂SCH₃ (S)-isomer | H | H | C—I | CH | CH | CH | C—CH₃ | CH | C—(A)r—Q | C—Cl | CH | CH₂ | 1 | Q34 | CF₃ | CF₃ | — | — | |
| 7-75 | CH(CH₃)CH₂SCH₃ (S)-isomer | H | H | C—SCH₃ | CH | CH | CH | C—CH₃ | CH | C—(A)r—Q | C—Cl | CH | CH₂ | 1 | Q34 | CF₃ | CF₃ | — | — | |
| 7-76 | CH(CH₃)CH₂SCH₃ (S)-isomer | H | H | C—Cl | CH | CH | CH | C—Cl | CH | C—(A)r—Q | C—Cl | CH | CH₂ | 1 | Q8 | CF₃ | H | H | — | |
| 7-77 | CH(CH₃)CH₂SCH₃ (S)-isomer | H | H | C—Cl | CH | CH | CH | C—Cl | CH | C—(A)r—Q | C—Cl | CH | CH₂ | 1 | Q34 | CF₃ | H | — | — | |
| 7-78 | CH(CH₃)CH₂SCH₃ (S)-isomer | H | H | C—Cl | CH | CH | CH | C—Cl | CH | C—(A)r—Q | C—Cl | CH | CH₂ | 1 | Q8 | CF₃ | H | CF₃ | — | |
| 7-79 | CH(CH₃)CH₂SCH₃ (S)-isomer | H | H | C—Br | CH | CH | CH | C—Cl | CH | C—(A)r—Q | C—Cl | CH | CH₂ | 1 | Q8 | CF₃ | H | CF₃ | — | |
| 7-80 | CH(CH₃)CH₂SCH₃ (S)-isomer | H | H | C—SO₂CH₃ | CH | CH | CH | C—Cl | CH | C—(A)r—Q | C—Cl | CH | CH₂ | 1 | Q8 | CF₃ | H | CF₃ | — | |
| 7-81 | CH(CH₃)CH₂SCH₃ (S)-isomer | H | H | C—Cl | CH | CH | CH | C—Cl | CH | C—(A)r—Q | C—Cl | CH | CH₂ | 1 | Q34 | CF₃ | CF₃ | — | — | |
| 7-82 | CH(CH₃)CH₂SCH₃ (S)-isomer | H | H | C—Br | CH | CH | CH | C—Cl | CH | C—(A)r—Q | C—Cl | CH | CH₂ | 1 | Q34 | CF₃ | CF₃ | — | — | |
| 7-83 | CH(CH₃)CH₂SCH₃ (S)-isomer | H | H | C—SO₂CH₃ | CH | CH | CH | C—Cl | CH | C—(A)r—Q | C—Cl | CH | CH₂ | 1 | Q34 | CF₃ | CF₃ | — | — | |
| 7-84 | CH(CH₃)CH₂SCH₃ (S)-isomer | H | H | C—Cl | CH | CH | CH | C—Cl | CH | C—(A)r—Q | C—Cl | CH | CH₂ | 1 | Q34 | CF₂CHF₂ | CF₃ | — | — | |
| 7-85 | CH(CH₃)CH₂SCH₃ (S)-isomer | H | H | C—Cl | CH | CH | CH | C—Cl | CH | C—(A)r—Q | C—Cl | CH | CH₂ | 1 | Q34 | C₂F₅ | CF₃ | — | — | |
| 7-86 | CH(CH₃)CH₂SCH₃ (S)-isomer | H | H | C—Cl | CH | CH | CH | C—Cl | CH | C—(A)r—Q | C—Cl | CH | CH₂ | 1 | Q34 | CF₃ | CF₂CHF₂ | — | — | |
| 7-87 | CH(CH₃)CH₂SCH₃ (S)-isomer | H | H | C—Cl | CH | CH | CH | C—CH₃ | CH | C—(A)r—Q | C—CF₃ | CH | CH₂ | 1 | Q8 | CF₃ | H | H | — | |
| 7-88 | CH(CH₃)CH₂SCH₃ (S)-isomer | H | H | C—Br | CH | CH | CH | C—CH₃ | CH | C—(A)r—Q | C—CF₃ | CH | CH₂ | 1 | Q8 | CF₃ | H | H | — | |
| 7-89 | CH(CH₃)CH₂SCH₃ (S)-isomer | H | H | C—I | CH | CH | CH | C—CH₃ | CH | C—(A)r—Q | C—CF₃ | CH | CH₂ | 1 | Q8 | CF₃ | H | H | — | |
| 7-90 | CH(CH₃)CH₂SCH₃ (S)-isomer | H | H | C—SCH₃ | CH | CH | CH | C—CH₃ | CH | C—(A)r—Q | C—CF₃ | CH | CH₂ | 1 | Q8 | CF₃ | H | H | — | |
| 7-91 | CH(CH₃)CH₂SCH₃ (S)-isomer | H | H | C—SO₂CH₃ | CH | CH | CH | C—CH₃ | CH | C—(A)r—Q | C—CF₃ | CH | CH₂ | 1 | Q8 | CF₃ | H | H | — | |
| 7-92 | CH(CH₃)CH₂SCH₃ (S)-isomer | H | H | C—Cl | CH | CH | CH | C—CH₃ | CH | C—(A)r—Q | C—CF₃ | CH | CH₂ | 1 | Q34 | CF₃ | H | — | — | |
| 7-93 | CH(CH₃)CH₂SCH₃ (S)-isomer | H | H | C—Br | CH | CH | CH | C—CH₃ | CH | C—(A)r—Q | C—CF₃ | CH | CH₂ | 1 | Q34 | CF₃ | H | — | — | |
| 7-94 | CH(CH₃)CH₂SCH₃ (S)-isomer | H | H | C—I | CH | CH | CH | C—CH₃ | CH | C—(A)r—Q | C—CF₃ | CH | CH₂ | 1 | Q34 | CF₃ | H | — | — | |
| 7-95 | CH(CH₃)CH₂SCH₃ (S)-isomer | H | H | C—SCH₃ | CH | CH | CH | C—CH₃ | CH | C—(A)r—Q | C—CF₃ | CH | CH₂ | 1 | Q34 | CF₃ | H | — | — | |
| 7-96 | CH(CH₃)CH₂SCH₃ (S)-isomer | H | H | C—SO₂CH₃ | CH | CH | CH | C—CH₃ | CH | C—(A)r—Q | C—CF₃ | CH | CH₂ | 1 | Q34 | CF₃ | H | — | — | |
| 7-97 | CH(CH₃)CH₂SCH₃ (S)-isomer | H | H | C—Cl | CH | CH | CH | C—CH₃ | CH | C—(A)r—Q | C—CF₃ | CH | CH₂ | 1 | Q8 | CF₃ | H | CF₃ | — | |
| 7-98 | CH(CH₃)CH₂SCH₃ (S)-isomer | H | H | C—Cl | CH | CH | CH | C—CH₃ | CH | C—(A)r—Q | C—CF₃ | CH | CH₂ | 1 | Q34 | CF₃ | CF₃ | — | — | |
| 7-99 | CH(CH₃)CH₂SCH₃ (S)-isomer | H | H | C—Cl | CH | CH | CH | C—Cl | CH | C—(A)r—Q | C—CF₃ | CH | CH₂ | 1 | Q8 | CF₃ | H | H | — | |
| 7-100 | CH(CH₃)CH₂SCH₃ (S)-isomer | H | H | C—Br | CH | CH | CH | C—Cl | CH | C—(A)r—Q | C—CF₃ | CH | CH₂ | 1 | Q8 | CF₃ | H | H | — | |
| 7-101 | CH(CH₃)CH₂SCH₃ (S)-isomer | H | H | C—I | CH | CH | CH | C—Cl | CH | C—(A)r—Q | C—CF₃ | CH | CH₂ | 1 | Q8 | CF₃ | H | H | — | |
| 7-102 | CH(CH₃)CH₂SCH₃ (S)-isomer | H | H | C—SCH₃ | CH | CH | CH | C—Cl | CH | C—(A)r—Q | C—CF₃ | CH | CH₂ | 1 | Q8 | CF₃ | H | H | — | |
| 7-103 | CH(CH₃)CH₂SCH₃ (S)-isomer | H | H | C—SO₂CH₃ | CH | CH | CH | C—Cl | CH | C—(A)r—Q | C—CF₃ | CH | CH₂ | 1 | Q8 | CF₃ | H | H | — | |
| 7-104 | CH(CH₃)CH₂SCH₃ (S)-isomer | H | H | C—Cl | CH | CH | CH | C—Cl | CH | C—(A)r—Q | C—CF₃ | CH | CH₂ | 1 | Q34 | CF₃ | H | — | — | |
| 7-105 | CH(CH₃)CH₂SCH₃ (S)-isomer | H | H | C—Br | CH | CH | CH | C—Cl | CH | C—(A)r—Q | C—CF₃ | CH | CH₂ | 1 | Q34 | CF₃ | H | — | — | |
| 7-106 | CH(CH₃)CH₂SCH₃ (S)-isomer | H | H | C—I | CH | CH | CH | C—Cl | CH | C—(A)r—Q | C—CF₃ | CH | CH₂ | 1 | Q34 | CF₃ | H | — | — | |
| 7-107 | CH(CH₃)CH₂SCH₃ (S)-isomer | H | H | C—SCH₃ | CH | CH | CH | C—Cl | CH | C—(A)r—Q | C—CF₃ | CH | CH₂ | 1 | Q34 | CF₃ | H | — | — | |
| 7-108 | CH(CH₃)CH₂SCH₃ (S)-isomer | H | H | C—SO₂CH₃ | CH | CH | CH | C—Cl | CH | C—(A)r—Q | C—CF₃ | CH | CH₂ | 1 | Q34 | CF₃ | H | — | — | |
| 7-109 | CH(CH₃)CH₂SCH₃ (S)-isomer | H | H | C—I | CH | CH | CH | C—Cl | CH | C—(A)r—Q | C—CF₃ | CH | CH₂ | 1 | Q8 | CF₃ | H | CF₃ | — | |
| 7-110 | CH(CH₃)CH₂SCH₃ (S)-isomer | H | H | C—I | CH | CH | CH | C—Cl | CH | C—(A)r—Q | C—CF₃ | CH | CH₂ | 1 | Q34 | CF₃ | CF₃ | — | — | |
| 7-111 | CH(CH₃)CH₂SCH₃ (S)-isomer | H | H | C—Cl | CH | CH | CH | C—CH₃ | CH | C—(A)r—Q | C—F | CH | CH(CH₃) | 1 | Q8 | CF₃ | H | CF₃ | — | |
| 7-112 | CH(CH₃)CH₂SCH₃ (S)-isomer | H | H | C—Br | CH | CH | CH | C—CH₃ | CH | C—(A)r—Q | C—F | CH | CH(CH₃) | 1 | Q8 | CF₃ | H | CF₃ | — | |
| 7-113 | CH(CH₃)CH₂SCH₃ (S)-isomer | H | H | C—I | CH | CH | CH | C—CH₃ | CH | C—(A)r—Q | C—F | CH | CH(CH₃) | 1 | Q8 | CF₃ | H | CF₃ | — | |
| 7-114 | CH(CH₃)CH₂SCH₃ (S)-isomer | H | H | C—SO₂CH₃ | CH | CH | CH | C—CH₃ | CH | C—(A)r—Q | C—F | CH | CH(CH₃) | 1 | Q8 | CF₃ | H | CF₃ | — | |
| 7-115 | CH(CH₃)CH₂SCH₃ (S)-isomer | H | H | C—Cl | CH | CH | CH | C—CH₃ | CH | C—(A)r—Q | C—F | CH | CH(CH₃) | 1 | Q34 | CF₃ | CF₃ | — | — | |
| 7-116 | CH(CH₃)CH₂SCH₃ (S)-isomer | H | H | C—Br | CH | CH | CH | C—CH₃ | CH | C—(A)r—Q | C—F | CH | CH(CH₃) | 1 | Q34 | CF₃ | CF₃ | — | — | |
| 7-117 | CH(CH₃)CH₂SCH₃ (S)-isomer | H | H | C—I | CH | CH | CH | C—CH₃ | CH | C—(A)r—Q | C—F | CH | CH(CH₃) | 1 | Q34 | CF₃ | CF₃ | — | — | |
| 7-118 | CH(CH₃)CH₂SCH₃ (S)-isomer | H | H | C—SO₂CH₃ | CH | CH | CH | C—CH₃ | CH | C—(A)r—Q | C—F | CH | CH(CH₃) | 1 | Q34 | CF₃ | CF₃ | — | — | |
| 7-119 | CH(CH₃)CH₂SCH₃ (S)-isomer | H | H | C—Cl | CH | CH | CH | C—Cl | CH | C—(A)r—Q | C—F | CH | CH(CH₃) | 1 | Q8 | CF₃ | H | CF₃ | — | |
| 7-120 | CH(CH₃)CH₂SO₂CH₃ (S)-isomer | H | H | C—Cl | CH | CH | CH | C—Cl | CH | C—(A)r—Q | C—F | CH | CH(CH₃) | 1 | Q8 | CF₃ | H | CF₃ | — | |
| 7-121 | CH(CH₃)CH₂SCH₃ (S)-isomer | H | H | C—I | CH | CH | CH | C—Cl | CH | C—(A)r—Q | C—F | CH | CH(CH₃) | 1 | Q8 | CF₃ | H | CF₃ | — | |
| 7-122 | CH(CH₃)CH₂SCH₃ (S)-isomer | H | H | C—SO₂CH₃ | CH | CH | CH | C—Cl | CH | C—(A)r—Q | C—F | CH | CH(CH₃) | 1 | Q8 | CF₃ | H | CF₃ | — | |
| 7-123 | CH(CH₃)CH₂SCH₃ (S)-isomer | H | H | C—Cl | CH | CH | CH | C—Cl | CH | C—(A)r—Q | C—F | CH | CH(CH₃) | 1 | Q34 | CF₃ | CF₃ | — | — | |
| 7-124 | CH(CH₃)CH₂SO₂CH₃ (S)-isomer | H | H | C—Cl | CH | CH | CH | C—Cl | CH | C—(A)r—Q | C—F | CH | CH(CH₃) | 1 | Q34 | CF₃ | CF₃ | — | — | |
| 7-125 | CH(CH₃)CH₂SCH₃ (S)-isomer | H | H | C—I | CH | CH | CH | C—Cl | CH | C—(A)r—Q | C—F | CH | CH(CH₃) | 1 | Q34 | CF₃ | CF₃ | — | — | |
| 7-126 | CH(CH₃)CH₂SCH₃ (S)-isomer | H | H | C—SO₂CH₃ | CH | CH | CH | C—Cl | CH | C—(A)r—Q | C—F | CH | CH(CH₃) | 1 | Q34 | CF₃ | CF₃ | — | — | |
| 7-127 | CH(CH₃)CH₂SCH₃ (S)-isomer | H | H | C—Cl | CH | CH | CH | C—CH₃ | CH | C—(A)r—Q | C—Cl | CH | CH(CH₃) | 1 | Q8 | CF₃ | H | H | — | |
| 7-128 | CH(CH₃)CH₂SCH₃ (S)-isomer | H | H | C—Cl | CH | CH | CH | C—CH₃ | CH | C—(A)r—Q | C—Cl | CH | CH(CH₃) | 1 | Q34 | CF₃ | H | — | — | |
| 7-129 | CH(CH₃)CH₂SCH₃ (S)-isomer | H | H | C—Cl | CH | CH | CH | C—CH₃ | CH | C—(A)r—Q | C—Cl | CH | CH(CH₃) | 1 | Q8 | CF₃ | H | CF₃ | — | |
| 7-130 | CH(CH₃)CH₂SCH₃ (S)-isomer | H | H | C—I | CH | CH | CH | C—CH₃ | CH | C—(A)r—Q | C—Cl | CH | CH(CH₃) | 1 | Q8 | CF₃ | H | CF₃ | — | |
| 7-131 | CH(CH₃)CH₂SCH₃ (S)-isomer | H | H | C—SCH₃ | CH | CH | CH | C—CH₃ | CH | C—(A)r—Q | C—Cl | CH | CH(CH₃) | 1 | Q8 | CF₃ | H | CF₃ | — | |
| 7-132 | CH(CH₃)CH₂SCH₃ (S)-isomer | H | H | C—Cl | CH | CH | CH | C—CH₃ | CH | C—(A)r—Q | C—Cl | CH | CH(CH₃) | 1 | Q34 | CF₃ | CF₃ | — | — | |
| 7-133 | CH(CH₃)CH₂SCH₃ (S)-isomer | H | H | C—I | CH | CH | CH | C—CH₃ | CH | C—(A)r—Q | C—Cl | CH | CH(CH₃) | 1 | Q34 | CF₃ | CF₃ | — | — | |
| 7-134 | CH(CH₃)CH₂SCH₃ (S)-isomer | H | H | C—SCH₃ | CH | CH | CH | C—CH₃ | CH | C—(A)r—Q | C—Cl | CH | CH(CH₃) | 1 | Q34 | CF₃ | CF₃ | — | — | |
| 7-135 | CH(CH₃)CH₂SCH₃ (S)-isomer | H | H | C—Cl | CH | CH | CH | C—Cl | CH | C—(A)r—Q | C—Cl | CH | CH(CH₃) | 1 | Q8 | CF₃ | H | H | — | |
| 7-136 | CH(CH₃)CH₂SCH₃ (S)-isomer | H | H | C—Cl | CH | CH | CH | C—Cl | CH | C—(A)r—Q | C—Cl | CH | CH(CH₃) | 1 | Q34 | CF₃ | H | — | — | |
| 7-137 | CH(CH₃)CH₂SCH₃ (S)-isomer | H | H | C—Cl | CH | CH | CH | C—Cl | CH | C—(A)r—Q | C—Cl | CH | CH(CH₃) | 1 | Q8 | CF₃ | H | CF₃ | — | |
| 7-138 | CH(CH₃)CH₂SCH₃ (S)-isomer | H | H | C—SCH₃ | CH | CH | CH | C—Cl | CH | C—(A)r—Q | C—Cl | CH | CH(CH₃) | 1 | Q8 | CF₃ | H | CF₃ | — | |
| 7-139 | CH(CH₃)CH₂SCH₃ (S)-isomer | H | H | C—SO₂CH₃ | CH | CH | CH | C—Cl | CH | C—(A)r—Q | C—Cl | CH | CH(CH₃) | 1 | Q8 | CF₃ | H | CF₃ | — | |
| 7-140 | CH(CH₃)CH₂SCH₃ (S)-isomer | H | H | C—Cl | CH | CH | CH | C—Cl | CH | C—(A)r—Q | C—Cl | CH | CH(CH₃) | 1 | Q34 | CF₃ | CF₃ | — | — | |
| 7-141 | CH(CH₃)CH₂SCH₃ (S)-isomer | H | H | C—SCH₃ | CH | CH | CH | C—Cl | CH | C—(A)r—Q | C—Cl | CH | CH(CH₃) | 1 | Q34 | CF₃ | CF₃ | — | — | |
| 7-142 | CH(CH₃)CH₂SCH₃ (S)-isomer | H | H | C—SO₂CH₃ | CH | CH | CH | C—Cl | CH | C—(A)r—Q | C—Cl | CH | CH(CH₃) | 1 | Q34 | CF₃ | CF₃ | — | — | |
| 7-143 | CH(CH₃)CH₂SCH₃ (S)-isomer | H | H | C—Cl | CH | CH | CH | C—Cl | CH | C—(A)r—Q | C—Cl | CH | CH(CH₃) | 1 | Q34 | CF₂CHF₂ | CF₃ | — | — | |
| 7-144 | CH(CH₃)CH₂SCH₃ (S)-isomer | H | H | C—Cl | CH | CH | CH | C—Cl | CH | C—(A)r—Q | C—Cl | CH | CH(CH₃) | 1 | Q34 | C₂F₅ | CF₃ | — | — | |
| 7-145 | CH(CH₃)CH₂SCH₃ (S)-isomer | H | H | C—Cl | CH | CH | CH | C—Cl | CH | C—(A)r—Q | C—Cl | CH | CH(CH₃) | 1 | Q34 | CF₃ | CF₂CHF₂ | — | — | |
| 7-146 | CH(CH₃)CH₂SCH₃ (S)-isomer | H | H | C—Cl | CH | CH | CH | C—CH₃ | N | C—(A)r—Q | C—F | CH | CH₂ | 1 | Q8 | CF₃ | H | H | — | |
| 7-147 | CH(CH₃)CH₂SCH₃ (S)-isomer | H | H | C—Cl | CH | CH | CH | C—CH₃ | N | C—(A)r—Q | C—F | CH | CH₂ | 1 | Q34 | CF₃ | H | — | — | |
| 7-148 | CH(CH₃)CH₂SCH₃ (S)-isomer | H | H | C—I | CH | CH | CH | C—CH₃ | N | C—(A)r—Q | C—F | CH | CH₂ | 1 | Q8 | C₂F₅ | H | H | — | |
| 7-149 | CH(CH₃)CH₂SCH₃ (S)-isomer | H | H | C—I | CH | CH | CH | C—CH₃ | N | C—(A)r—Q | C—F | CH | CH₂ | 1 | Q34 | C₂F₅ | H | — | — | |
| 7-150 | CH(CH₃)CH₂SCH₃ (S)-isomer | H | H | C—Cl | CH | CH | CH | C—CH₃ | N | C—(A)r—Q | C—F | CH | CH₂ | 1 | Q8 | CF₃ | H | CF₃ | — | logP (acid) 4.13 |
| 7-151 | CH(CH₃)CH₂SOCH₃ (S)-isomer | H | H | C—Cl | CH | CH | CH | C—CH₃ | N | C—(A)r—Q | C—F | CH | CH₂ | 1 | Q8 | CF₃ | H | | | |
| 7-152 | CH(CH₃)CH₂SO₂CH₃ (S)-isomer | H | H | C—Cl | CH | CH | CH | C—CH₃ | N | C—(A)r—Q | C—F | CH | CH₂ | 1 | Q8 | CF₃ | H | | | |
| 7-153 | CH(CH₃)CH₂SCH₃ (S)-isomer | H | H | C—Cl | CH | CH | CH | C—CH₃ | N | C—(A)r—Q | C—F | CH | CH₂ | 1 | Q8 | C₂F₅ | H | | | |
| 7-154 | CH(CH₃)CH₂SOCH₃ (S)-isomer | H | H | C—Cl | CH | CH | CH | C—CH₃ | N | C—(A)r—Q | C—F | CH | CH₂ | 1 | Q8 | C₂F₅ | H | | | |
| 7-155 | CH(CH₃)CH₂SO₂CH₃ (S)-isomer | H | H | C—Cl | CH | CH | CH | C—CH₃ | N | C—(A)r—Q | C—F | CH | CH₂ | 1 | Q8 | C₂F₅ | H | | | |
| 7-156 | CH(CH₃)CH₂SCH₃ (S)-isomer | H | H | C—Cl | CH | CH | CH | C—CH₃ | N | C—(A)r—Q | C—F | CH | CH₂ | 1 | Q8 | C₂F₅ | H | | | |
| 7-157 | CH(CH₃)CH₂SOCH₃ (S)-isomer | H | H | C—Cl | CH | CH | CH | C—CH₃ | N | C—(A)r—Q | C—F | CH | CH₂ | 1 | Q8 | C₂F₅ | H | | | |
| 7-158 | CH(CH₃)CH₂SO₂CH₃ (S)-isomer | H | H | C—Cl | CH | CH | CH | C—CH₃ | N | C—(A)r—Q | C—F | CH | CH₂ | 1 | Q8 | C₂F₅ | H | | | |
| 7-159 | CH(CH₃)CH₂SCH₃ (S)-isomer | H | H | C—Cl | CH | CH | CH | C—CH₃ | N | C—(A)r—Q | C—F | CH | CH₂ | 1 | Q34 | CF₃ | CF₃ | | | |
| 7-160 | CH(CH₃)CH₂SOCH₃ (S)-isomer | H | H | C—Cl | CH | CH | CH | C—CH₃ | N | C—(A)r—Q | C—F | CH | CH₂ | 1 | Q34 | CF₃ | CF₃ | | | |
| 7-161 | CH(CH₃)CH₂SO₂CH₃ (S)-isomer | H | H | C—Cl | CH | CH | CH | C—CH₃ | N | C—(A)r—Q | C—F | CH | CH₂ | 1 | Q34 | CF₃ | CF₃ | | | |
| 7-162 | CH(CH₃)CH₂SCH₃ (S)-isomer | H | H | C—I | CH | CH | CH | C—CH₃ | N | C—(A)r—Q | C—Cl | CH | CH₂ | 1 | Q8 | CF₃ | H | | | |
| 7-163 | CH(CH₃)CH₂SCH₃ (S)-isomer | H | H | C—Cl | CH | CH | CH | C—CH₃ | N | C—(A)r—Q | C—Cl | CH | CH₂ | 1 | Q8 | CF₃ | H | | | |
| 7-164 | CH(CH₃)CH₂SCH₃ (S)-isomer | H | H | C—Cl | CH | CH | CH | C—CH₃ | N | C—(A)r—Q | C—Cl | CH | CH₂ | 1 | Q34 | CF₃ | CF₃ | | | |
| 7-165 | CH(CH₃)CH₂SCH₃ (S)-isomer | H | H | C—SO₂CH₃ | CH | CH | CH | C—CH₃ | N | C—(A)r—Q | C—Cl | CH | CH₂ | 1 | Q8 | CF₃ | H | | | |
| 7-166 | CH(CH₃)CH₂SCH₃ (S)-isomer | H | H | C—SO₂CH₃ | CH | CH | CH | C—CH₃ | N | C—(A)r—Q | C—Cl | CH | CH₂ | 1 | Q34 | CF₃ | CF₃ | | | |

TABLE 8

| No. | R¹ | R² | R³ | W¹ | W² | W³ | W⁴ | W⁵ |
|---|---|---|---|---|---|---|---|---|
| 8-1 | CH(CH₃)₂ | H | H | CH | CH | CH | CH | CH |
| 8-2 | CH(CH₃)₂ | H | H | CH | CH | CH | CH | CH |
| 8-3 | CH(CH₃)CH₂SCH₃ (S)-isomer | H | H | CH | CH | CH | CH | CH |
| 8-4 | CH(CH₃)CH₂SOCH₃ (S)-isomer | H | H | CH | CH | CH | CH | CH |
| 8-5 | CH(CH₃)₂ | H | H | CH | CH | CH | CH | CH |
| 8-6 | CH(CH₃)₂ | H | H | C—Cl | CH | CH | CH | CH |
| 8-7 | CH(CH₃)CH₂SCH₃ (S)-isomer | H | H | C—Cl | CH | CH | CH | CH |
| 8-8 | CH(CH₃)CH₂SCH₃ (S)-isomer | H | H | C—Cl | CH | CH | CH | CH |
| 8-9 | CH(CH₃)CH₂SOCH₃ (S)-isomer | H | H | C—Cl | CH | CH | CH | CH |
| 8-10 | CH(CH₃)CH₂SOCH₃ (S)-isomer | H | H | C—Cl | CH | CH | CH | CH |
| 8-11 | CH(CH₃)CH₂SO₂CH₃ (S)-isomer | H | H | C—Cl | CH | CH | CH | CH |
| 8-12 | CH(CH₃)CH₂SO₂CH₃ (S)-isomer | H | H | C—Cl | CH | CH | CH | CH |
| 8-13 | CH(CH₃)CH₂SCH₃ (S)-isomer | H | H | C—Cl | CH | CH | CH | CH |
| 8-14 | CH(CH₃)CH₂SCH₃ (S)-isomer | H | H | C—Cl | CH | CH | CH | CH |
| 8-15 | CH(CH₃)CH₂SCH₃ (S)-isomer | H | H | C—Cl | CH | CH | CH | CH |
| 8-16 | CH(CH₃)CH₂SCH₃ (S)-isomer | H | H | C—Br | CH | CH | CH | CH |
| 8-17 | CH(CH₃)CH₂SCH₃ (S)-isomer | H | H | C—Br | CH | CH | CH | CH |
| 8-18 | CH(CH₃)CH₂SCH₃ (S)-isomer | H | H | C—Br | CH | CH | CH | CH |
| 8-19 | CH(CH₃)₂ | H | H | C—OSO₂CH₃ | CH | CH | CH | CH |
| 8-20 | CH(CH₃)₂ | H | H | C—OSO₂C₂H₅ | CH | CH | CH | CH |
| 8-21 | CH(CH₃)₂ | H | H | C—OSO₂CF₃ | CH | CH | CH | CH |
| 8-22 | CH(CH₃)₂ | H | H | C—OCH₂CH═CH₂ | CH | CH | CH | CH |
| 8-23 | CH(CH₃)₂ | H | H | C—OCH₂C≡CH | CH | CH | CH | CH |
| 8-24 | CH(CH₃)₂ | H | H | C-cyclohexyl | CH | CH | CH | CH |
| 8-25 | CH(CH₃)₂ | H | H | C—O-cyclopentyl | CH | CH | CH | CH |
| 8-26 | CH(CH₃)₂ | H | H | C—O-phenyl | CH | CH | CH | CH |
| 8-27 | CH(CH₃)₂ | H | H | C—CH₂O-phenyl | CH | CH | CH | CH |
| 8-28 | CH(CH₃)₂ | H | H | C—CH₂S-phenyl | CH | CH | CH | CH |
| 8-29 | CH(CH₃)₂ | H | H | C—S-phenyl | CH | CH | CH | CH |
| 8-30 | CH(CH₃)₂ | H | H | C—CH₂-phenyl | CH | CH | CH | CH |
| 8-31 | CH(CH₃)₂ | H | H | C-pyridin-2-yl | CH | CH | CH | CH |
| 8-32 | CH(CH₃)₂ | H | H | C-pyridin-3-yl | CH | CH | CH | CH |
| 8-33 | CH(CH₃)₂ | H | H | C-pyridin-4-yl | CH | CH | CH | CH |
| 8-34 | CH(CH₃)₂ | H | H | C-pyrazole | CH | CH | CH | CH |
| 8-35 | CH(CH₃)₂ | H | H | C—O-pyridin-2-y | CH | CH | CH | CH |
| 8-36 | CH(CH₃)₂ | H | H | C—CH₂O-pyridin-2-y | CH | CH | CH | CH |
| 8-37 | CH(CH₃)₂ | H | H | C—S-pyridin-2-y | CH | CH | CH | CH |
| 8-38 | CH(CH₃)CH₂SCH₃ (S)-isomer | H | H | C—Cl | CH | CH | CH | CH |
| 8-39 | CH(CH₃)CH₂SCH₃ (S)-isomer | H | H | C—Cl | CH | CH | CH | CH |
| 8-40 | CH(CH₃)CH₂SCH₃ (S)-isomer | H | H | C—NO₂ | CH | CH | CH | CH |
| 8-41 | CH(CH₃)CH₂SCH₃ (S)-isomer | H | H | C—NO₂ | CH | CH | CH | CH |
| 8-42 | CH(CH₃)CH₂SCH₃ (S)-isomer | H | H | C—Cl | CH | CH | CH | CH |
| 8-43 | CH(CH₃)CH₂SCH₃ (S)-isomer | H | H | C—Cl | CH | CH | CH | CH |
| 8-44 | CH(CH₃)CH₂SCH₃ (S)-isomer | H | H | C—NO₂ | CH | CH | CH | CH |
| 8-45 | CH(CH₃)CH₂SCH₃ (S)-isomer | H | H | C—NO₂ | CH | CH | CH | CH |

| No. | W⁶ | W⁷ | W⁸ | W⁹ | A | r | Q | R⁴ | R⁵ | R⁶ | R⁷ | Physical property |
|---|---|---|---|---|---|---|---|---|---|---|---|---|
| 8-1 | CH | C—(A)r—Q | CH | CH | CH₂ | 1 | Q8 | CF₃ | H | CF₃ | — | mp 176-178° C. |
| 8-2 | CH | C—(A)r—Q | CH | CH | CH₂ | 1 | Q8 | C₂F₅ | C₂F₅ | H | — | NMR |
| 8-3 | CH | C—(A)r—Q | CH | CH | CH₂ | 1 | Q8 | C₂F₅ | C₂F₅ | H | — | NMR |
| 8-4 | CH | C—(A)r—Q | CH | CH | CH₂ | 1 | Q8 | C₂F₅ | C₂F₅ | H | — | NMR |
| 8-5 | CH | C—(A)r—Q | CH | CH | CH₂ | 1 | Q34 | CF₃ | CF₃ | — | — | |
| 8-6 | CH | C—(A)r—Q | CH | CH | CH₂ | 1 | Q8 | C₂F₅ | C₂F₅ | H | — | NMR |
| 8-7 | CH | C—(A)r—Q | CH | CH | CH₂ | 1 | Q8 | CF₃ | H | CF₃ | — | NMR |
| 8-8 | CH | C—(A)r—Q | CH | CH | CH₂ | 1 | Q8 | C₂F₅ | C₂F₅ | H | — | NMR |
| 8-9 | CH | C—(A)r—Q | CH | CH | CH₂ | 1 | Q8 | CF₃ | H | CF₃ | — | |
| 8-10 | CH | C—(A)r—Q | CH | CH | CH₂ | 1 | Q8 | C₂F₅ | C₂F₅ | H | — | |
| 8-11 | CH | C—(A)r—Q | CH | CH | CH₂ | 1 | Q8 | CF₃ | H | CF₃ | — | |
| 8-12 | CH | C—(A)r—Q | CH | CH | CH₂ | 1 | Q8 | C₂F₅ | C₂F₅ | H | — | |
| 8-13 | CH | C—(A)r—Q | CH | CH | CH₂ | 1 | Q34 | CF₃ | CF₃ | — | — | NMR |
| 8-14 | CH | C—(A)r—Q | CH | CH | CH₂ | 1 | Q34 | C₂F₅ | CF₃ | — | — | NMR |
| 8-15 | CH | C—(A)r—Q | CH | CH | CH₂ | 1 | Q34 | C₂F₅ | C₂F₅ | — | — | NMR |
| 8-16 | CH | C—(A)r—Q | CH | CH | CH₂ | 1 | Q8 | CF₃ | H | CF₃ | — | NMR |
| 8-17 | CH | C—(A)r—Q | CH | CH | CH₂ | 1 | Q8 | C₂F₅ | C₂F₅ | H | — | |
| 8-18 | CH | C—(A)r—Q | CH | CH | CH₂ | 1 | Q34 | C₂F₅ | CF₃ | — | — | NMR |
| 8-19 | CH | C—(A)r—Q | CH | CH | CH₂ | 1 | Q8 | CF₃ | H | CF₃ | — | |
| 8-20 | CH | C—(A)r—Q | CH | CH | CH₂ | 1 | Q8 | CF₃ | H | CF₃ | — | |
| 8-21 | CH | C—(A)r—Q | CH | CH | CH₂ | 1 | Q8 | CF₃ | H | CF₃ | — | |
| 8-22 | CH | C—(A)r—Q | CH | CH | CH₂ | 1 | Q8 | CF₃ | H | CF₃ | — | |
| 8-23 | CH | C—(A)r—Q | CH | CH | CH₂ | 1 | Q8 | CF₃ | H | CF₃ | — | |
| 8-24 | CH | C—(A)r—Q | CH | CH | CH₂ | 1 | Q8 | CF₃ | H | CF₃ | — | |
| 8-25 | CH | C—(A)r—Q | CH | CH | CH₂ | 1 | Q8 | CF₃ | H | CF₃ | — | |
| 8-26 | CH | C—(A)r—Q | CH | CH | CH₂ | 1 | Q8 | CF₃ | H | CF₃ | — | |
| 8-27 | CH | C—(A)r—Q | CH | CH | CH₂ | 1 | Q8 | CF₃ | H | CF₃ | — | |
| 8-28 | CH | C—(A)r—Q | CH | CH | CH₂ | 1 | Q8 | CF₃ | H | CF₃ | — | |
| 8-29 | CH | C—(A)r—Q | CH | CH | CH₂ | 1 | Q8 | CF₃ | H | CF₃ | — | |
| 8-30 | CH | C—(A)r—Q | CH | CH | CH₂ | 1 | Q8 | CF₃ | H | CF₃ | — | |
| 8-31 | CH | C—(A)r—Q | CH | CH | CH₂ | 1 | Q8 | CF₃ | H | CF₃ | — | |
| 8-32 | CH | C—(A)r—Q | CH | CH | CH₂ | 1 | Q8 | CF₃ | H | CF₃ | — | |
| 8-33 | CH | C—(A)r—Q | CH | CH | CH₂ | 1 | Q8 | CF₃ | H | CF₃ | — | |
| 8-34 | CH | C—(A)r—Q | CH | CH | CH₂ | 1 | Q8 | CF₃ | H | CF₃ | — | |
| 8-35 | CH | C—(A)r—Q | CH | CH | CH₂ | 1 | Q8 | CF₃ | H | CF₃ | — | |
| 8-36 | CH | C—(A)r—Q | CH | CH | CH₂ | 1 | Q8 | CF₃ | H | CF₃ | — | |
| 8-37 | CH | C—(A)r—Q | CH | CH | CH₂ | 1 | Q8 | CF₃ | H | CF₃ | — | |
| 8-38 | CH | C—(A)r—Q | CH | CH | CH₂ | 2 | Q8 | CF₃ | H | CF₃ | — | |
| 8-39 | CH | C—(A)r—Q | CH | CH | CH₂ | 2 | Q34 | CF₃ | CF₃ | — | — | |
| 8-40 | CH | C—(A)r—Q | CH | CH | CH₂ | 2 | Q8 | CF₃ | H | CF₃ | — | |
| 8-41 | CH | C—(A)r—Q | CH | CH | CH₂ | 2 | Q34 | CF₃ | CF₃ | — | — | |
| 8-42 | CH | C—(A)r—Q | CH | CH | CH₂ | 3 | Q8 | CF₃ | H | CF₃ | — | |
| 8-43 | CH | C—(A)r—Q | CH | CH | CH₂ | 3 | Q34 | CF₃ | CF₃ | — | — | |
| 8-44 | CH | C—(A)r—Q | CH | CH | CH₂ | 3 | Q8 | CF₃ | H | CF₃ | — | |
| 8-45 | CH | C—(A)r—Q | CH | CH | CH₂ | 3 | Q34 | CF₃ | CF₃ | — | — | |

TABLE 9

| No. | R¹ | R² | R³ | W¹ | W² | W³ | W⁴ | W⁵ | W⁶ |
|---|---|---|---|---|---|---|---|---|---|
| 9-1 | CH(CH₃)CH₂SCH₃ (S)-isomer | H | H | C—F | C—F | CH | CH | C—CH₃ | CH |
| 9-2 | CH(CH₃)CH₂SCH₃ (S)-isomer | H | H | C—Cl | C—Cl | CH | CH | C—CH₃ | CH |
| 9-3 | CH(CH₃)CH₂SOCH₃ (S)-isomer | H | H | C—Cl | C—Cl | CH | CH | C—CH₃ | CH |
| 9-4 | CH(CH₃)CH₂SO₂CH₃ (S)-isomer | H | H | C—Cl | C—Cl | CH | CH | C—CH₃ | CH |
| 9-5 | CH(CH₃)CH₂SCH₃ (S)-isomer | H | H | C—Cl | C—Cl | CH | CH | C—CH₃ | CH |
| 9-6 | CH(CH₃)CH₂SOCH₃ (S)-isomer | H | H | C—Cl | C—Cl | CH | CH | C—CH₃ | CH |
| 9-7 | CH(CH₃)CH₂SO₂CH₃ (S)-isomer | H | H | C—Cl | C—Cl | CH | CH | C—CH₃ | CH |
| 9-8 | CH(CH₃)CH₂SCH₃ (S)-isomer | H | H | C—Cl | C—Cl | CH | CH | C—CH₃ | CH |
| 9-9 | CH(CH₃)CH₂SCH₃ (S)-isomer | H | H | C—F | C—CF₃ | CH | CH | C—CH₃ | CH |
| 9-10 | CH(CH₃)CH₂SCH₃ (S)-isomer | H | H | C—Br | C—F | CH | CH | C—CH₃ | N |
| 9-11 | CH(CH₃)CH₂SOCH₃ (S)-isomer | H | H | C—Br | C—F | CH | CH | C—CH₃ | N |
| 9-12 | CH(CH₃)CH₂SCH₃ (S)-isomer | H | H | C—Br | C—F | CH | CH | C—CH₃ | CH |
| 9-13 | CH(CH₃)CH₂SOCH₃ (S)-isomer | H | H | C—Br | C—F | CH | CH | C—CH₃ | CH |
| 9-14 | CH(CH₃)CH₂SCH₃ (S)-isomer | H | H | C—Cl | C—CF₃ | CH | CH | C—CH₃ | CH |
| 9-15 | CH(CH₃)CH₂SCH₃ (S)-isomer | H | H | C—Cl | NO₂ | CH | CH | C—CH₃ | CH |
| 9-16 | CH(CH₃)CH₂SOCH₃ (S)-isomer | H | H | C—Cl | C—CF₃ | CH | CH | C—CH₃ | CH |
| 9-17 | CH(CH₃)CH₂SO₂CH₃ (S)-isomer | H | H | C—Cl | C—CF₃ | CH | CH | C—CH₃ | CH |
| 9-18 | CH(CH₃)CH₂SCH₃ (S)-isomer | H | H | C—Cl | C—CF₃ | CH | CH | C—CH₃ | CH |
| 9-19 | CH(CH₃)CH₂SOCH₃ (S)-isomer | H | H | C—Cl | C—CF₃ | CH | CH | C—CH₃ | CH |
| 9-20 | CH(CH₃)CH₂SO₂CH₃ (S)-isomer | H | H | C—Cl | C—CF₃ | CH | CH | C—CH₃ | CH |
| 9-21 | CH(CH₃)CH₂SO₂CH₃ (S)-isomer | H | H | C—Br | C—F | CH | CH | C—CH₃ | N |
| 9-22 | CH(CH₃)CH₂SO₂CH₃ (S)-isomer | H | H | C—Br | C—F | CH | CH | C—CH₃ | CH |
| 9-23 | CH(CH₃)CH₂SCH₃ (S)-isomer | H | H | C—Cl | C—CF₃ | CH | CH | C—CH₃ | CH |
| 9-24 | CH(CH₃)CH₂SOCH₃ (S)-isomer | H | H | C—Cl | C—CF₃ | CH | CH | C—CH₃ | CH |
| 9-25 | CH(CH₃)CH₂SO₂CH₃ (S)-isomer | H | H | C—Cl | C—CF₃ | CH | CH | C—CH₃ | CH |
| 9-26 | CH(CH₃)CH₂SCH₃ (S)-isomer | H | H | C—Cl | C—CF₃ | CH | CH | C—CH₃ | N |

| No. | W⁷ | W⁸ | W⁹ | A | r | Q | R⁴ | R⁵ | R⁶ | R⁷ | Physical property |
|---|---|---|---|---|---|---|---|---|---|---|---|
| 9-1 | C—(A)r—Q | CH | CH | CH₂ | 1 | Q8 | CF₃ | H | CF₃ | — | |
| 9-2 | C—(A)r—Q | CH | CH | CH₂ | 1 | Q8 | CF₃ | H | CF₃ | — | NMR |
| 9-3 | C—(A)r—Q | CH | CH | CH₂ | 1 | Q8 | CF₃ | H | CF₃ | — | |
| 9-4 | C—(A)r—Q | CH | CH | CH₂ | 1 | Q8 | CF₃ | H | CF₃ | — | |
| 9-5 | C—(A)r—Q | CH | CH | CH₂ | 1 | Q34 | CF₃ | CF₃ | — | — | NMR |
| 9-6 | C—(A)r—Q | CH | CH | CH₂ | 1 | Q34 | CF₃ | CF₃ | — | — | |
| 9-7 | C—(A)r—Q | CH | CH | CH₂ | 1 | Q34 | CF₃ | CF₃ | — | — | |
| 9-8 | C—(A)r—Q | CH | CH | CH₂ | 1 | Q34 | C₂F₅ | CF₃ | — | — | NMR |
| 9-9 | C—(A)r—Q | CH | CH | CH₂ | 1 | Q8 | CF₃ | H | CF₃ | — | |
| 9-10 | C—(A)r—Q | CH | CH | CH₂ | 1 | Q8 | CF₃ | H | CF₃ | — | logP (neutral) 3.74 |
| 9-11 | C—(A)r—Q | CH | CH | CH₂ | 1 | Q8 | CF₃ | H | CF₃ | — | logP (acid) 2.51 |
| 9-12 | C—(A)r—Q | CH | CH | CH₂ | 1 | Q34 | C₂F₅ | CF₃ | — | — | logP (acid) 4.29 |
| 9-13 | C—(A)r—Q | CH | CH | CH₂ | 1 | Q34 | C₂F₅ | CF₃ | — | — | logP (acid) 2.96 |
| 9-14 | C—(A)r—Q | CH | CH | CH₂ | 1 | Q8 | CF₃ | H | CF₃ | — | logP (acid) 4.62 |
| 9-15 | C—(A)r—Q | CH | CH | CH₂ | 1 | Q34 | C₂F₅ | CF₃ | — | — | logP (acid) 4.27 |
| 9-16 | C—(A)r—Q | CH | CH | CH₂ | 1 | Q8 | CF₃ | H | CF₃ | — | |
| 9-17 | C—(A)r—Q | CH | CH | CH₂ | 1 | Q8 | CF₃ | H | CF₃ | — | logP (acid) 3.22 |
| 9-18 | C—(A)r—Q | CH | CH | CH₂ | 1 | Q34 | CF₃ | CF₃ | — | — | logP (acid) 4.30 |
| 9-19 | C—(A)r—Q | CH | CH | CH₂ | 1 | Q34 | CF₃ | CF₃ | — | — | logP (acid) 2.98 |
| 9-20 | C—(A)r—Q | CH | CH | CH₂ | 1 | Q34 | CF₃ | CF₃ | — | — | |
| 9-21 | C—(A)r—Q | CH | CH | CH₂ | 1 | Q8 | CF₃ | H | CF₃ | — | logP (acid) 3.27 |
| 9-22 | C—(A)r—Q | CH | CH | CH₂ | 1 | Q34 | C₂F₅ | CF₃ | — | — | logP (acid) 3.36 |
| 9-23 | C—(A)r—Q | CH | CH | CH₂ | 1 | Q34 | C₂F₅ | CF₃ | — | — | logP (acid) 4.63 |
| 9-24 | C—(A)r—Q | CH | CH | CH₂ | 1 | Q34 | C₂F₅ | CF₃ | — | — | logP (neutral) 3.13 |
| 9-25 | C—(A)r—Q | CH | CH | CH₂ | 1 | Q34 | C₂F₅ | CF₃ | — | — | logP (acid) 3.70 |
| 9-26 | C—(A)r—Q | CH | CH | CH₂ | 1 | Q8 | CF₃ | H | CF₃ | — | logP (acid) 4.25 |

TABLE 10

| No. | R¹ | R² | R³ | W¹ | W² | W³ | W⁴ | W⁵ |
|---|---|---|---|---|---|---|---|---|
| 10-1 | CH(CH₃)CH₂SCH₃ (S)-isomer | H | H | C—Cl | CH | C—Cl | CH | C—CH₃ |
| 10-2 | CH(CH₃)CH₂SOCH₃ (S)-isomer | H | H | C—Cl | CH | C—Cl | CH | C—CH₃ |
| 10-3 | CH(CH₃)CH₂SO₂CH₃ (S)-isomer | H | H | C—Cl | CH | C—Cl | CH | C—CH₃ |
| 10-4 | CH(CH₃)CH₂SCH₃ (S)-isomer | H | H | C—Cl | CH | C—Cl | CH | C—CH₃ |
| 10-5 | CH(CH₃)CH₂SCH₃ (S)-isomer | H | H | C—Cl | CH | C—Cl | CH | C—CH₃ |
| 10-6 | CH(CH₃)CH₂SCH₃ (S)-isomer | H | H | C—Cl | CH | C—Cl | CH | C—CH₃ |
| 10-7 | CH(CH₃)CH₂SCH₃ (S)-isomer | H | H | C—Cl | CH | C—Cl | CH | C—CH₃ |
| 10-8 | CH(CH₃)CH₂SCH₃ (S)-isomer | H | H | C—Cl | CH | C—Cl | CH | C—CH₃ |
| 10-9 | CH(CH₃)CH₂SCH₃ (S)-isomer | H | H | C—Cl | CH | C—Cl | CH | C—CH₃ |
| 10-10 | CH(CH₃)CH₂SCH₃ (S)-isomer | H | H | C—Cl | CH | C—Cl | CH | C—CH₃ |
| 10-11 | CH(CH₃)CH₂SOCH₃ (S)-isomer | H | H | C—Cl | CH | C—Cl | CH | C—CH₃ |
| 10-12 | CH(CH₃)CH₂SO₂CH₃ (S)-isomer | H | H | C—Cl | CH | C—Cl | CH | C—CH₃ |
| 10-13 | CH(CH₃)CH₂SCH₃ (S)-isomer | H | H | C—Cl | CH | C—F | CH | C—CH₃ |
| 10-14 | CH(CH₃)CH₂SOCH₃ (S)-isomer | H | H | C—Cl | CH | C—F | CH | C—CH₃ |
| 10-15 | CH(CH₃)CH₂SO₂CH₃ (S)-isomer | H | H | C—Cl | CH | C—F | CH | C—CH₃ |
| 10-16 | CH(CH₃)₂ | H | H | C—Br | CH | C—Br | CH | C—CH₃ |
| 10-17 | CH(CH₃)CH₂SCH₃ (S)-isomer | H | H | C—Br | CH | C—Br | CH | C—CH₃ |
| 10-18 | CH(CH₃)₂ | H | H | C—Br | CH | C—Br | CH | C—CH₃ |
| 10-19 | CH(CH₃)CH₂SCH₃ (S)-isomer | H | H | C—Br | CH | C—Br | CH | C—CH₃ |
| 10-20 | CH(CH₃)₂ | H | H | C—CH₃ | CH | C—CH₃ | CH | C—CH₃ |
| 10-21 | CH(CH₃)CH₂SCH₃ (S)-isomer | H | H | C—CH₃ | CH | C—CH₃ | CH | C—CH₃ |
| 10-22 | CH(CH₃)₂ | H | H | C—CH₃ | CH | C—CH₃ | CH | C—CH₃ |
| 10-23 | CH(CH₃)CH₂SCH₃ (S)-isomer | H | H | C—CH₃ | CH | C—CH₃ | CH | C—CH₃ |
| 10-24 | CH(CH₃)CH₂SCH₃ (S)-isomer | H | H | C—Cl | CH | C—Cl | CH | C—CH₃ |
| 10-25 | CH(CH₃)CH₂SCH₃ (S)-isomer | H | H | C—Br | CH | C—Br | CH | C—CH₃ |

| No. | W⁶ | W⁷ | W⁸ | W⁹ | A | r | Q | R⁴ | R⁵ | R⁶ | R⁷ | Physical property |
|---|---|---|---|---|---|---|---|---|---|---|---|---|
| 10-1 | N | C—(A)r—Q | CH | CH | CH₂ | 1 | Q8 | CF₃ | H | CF₃ | — | logP (acid) 4.18 |
| 10-2 | N | C—(A)r—Q | CH | CH | CH₂ | 1 | Q8 | CF₃ | H | CF₃ | — | logP (acid) 2.83 |
| 10-3 | N | C—(A)r—Q | CH | CH | CH₂ | 1 | Q8 | CF₃ | H | CF₃ | — | logP (neutral) |
| | | | | | | | | | | | | 3.10 |
| 10-4 | N | C—(A)r—Q | CH | CH | CH₂ | 1 | Q8 | CF₃ | H | H | — | |
| 10-5 | N | C—(A)r—Q | CH | CH | CH₂ | 1 | Q34 | C₂F₅ | H | — | — | |
| 10-6 | N | C—(A)r—Q | CH | CH | CH₂ | 1 | Q34 | CF₃ | CF₃ | — | — | |
| 10-7 | CH | C—(A)r—Q | CH | CH | CH₂ | 1 | Q8 | CF₃ | H | CF₃ | — | |
| 10-8 | CH | C—(A)r—Q | CH | CH | CH₂ | 1 | Q8 | C₂F₅ | H | C₂F₅ | — | |
| 10-9 | CH | C—(A)r—Q | CH | CH | CH₂ | 1 | Q34 | CF₃ | CF₃ | — | — | |
| 10-10 | CH | C—(A)r—Q | CH | CH | CH₂ | 1 | Q34 | C₂F₅ | CF₃ | — | — | logP (acid) 4.64 |
| 10-11 | CH | C—(A)r—Q | CH | CH | CH₂ | 1 | Q34 | C₂F₅ | CF₃ | — | — | logP (acid) 3.28 |
| 10-12 | CH | C—(A)r—Q | CH | CH | CH₂ | 1 | Q34 | C₂F₅ | CF₃ | — | — | logP (acid) 3.61 |
| 10-13 | CH | C—(A)r—Q | CH | CH | CH₂ | 1 | Q34 | C₂F₅ | CF₃ | — | — | NMR |
| 10-14 | CH | C—(A)r—Q | CH | CH | CH₂ | 1 | Q34 | C₂F₅ | CF₃ | — | — | logP (acid) 3.00 |
| 10-15 | CH | C—(A)r—Q | CH | CH | CH₂ | 1 | Q34 | C₂F₅ | CF₃ | — | — | logP (acid) 3.37 |
| 10-16 | CH | C—(A)r—Q | CH | CH | CH₂ | 1 | Q8 | CF₃ | H | CF₃ | — | |
| 10-17 | CH | C—(A)r—Q | CH | CH | CH₂ | 1 | Q8 | CF₃ | H | CF₃ | — | |
| 10-18 | CH | C—(A)r—Q | CH | CH | CH₂ | 1 | Q34 | CF₃ | CF₃ | — | — | |
| 10-19 | CH | C—(A)r—Q | CH | CH | CH₂ | 1 | Q34 | CF₃ | CF₃ | — | — | |
| 10-20 | CH | C—(A)r—Q | CH | CH | CH₂ | 1 | Q8 | CF₃ | H | CF₃ | — | |
| 10-21 | CH | C—(A)r—Q | CH | CH | CH₂ | 1 | Q8 | CF₃ | H | CF₃ | — | |
| 10-22 | CH | C—(A)r—Q | CH | CH | CH₂ | 1 | Q34 | CF₃ | CF₃ | — | — | |
| 10-23 | CH | C—(A)r—Q | CH | CH | CH₂ | 1 | Q34 | CF₃ | CF₃ | — | — | |
| 10-24 | N | C—(A)r—Q | N | CH | CH₂ | 1 | Q8 | CF₃ | H | CF₃ | — | |
| 10-25 | N | C—(A)r—Q | N | CH | CH₂ | 1 | Q8 | CF₃ | H | CF₃ | — | |

TABLE 11

| No. | R¹ | R² | R³ | W¹ | W² | W³ | W⁴ | W⁵ | W⁶ |
|---|---|---|---|---|---|---|---|---|---|
| 11-1 | CH(CH₃)CH₂SCH₃ (S)-isomer | H | H | C—I | CH | CH | C—F | C—CH₃ | N |
| 11-2 | CH(CH₃)CH₂SOCH₃ (S)-isomer | H | H | C—I | CH | CH | C—F | C—CH₃ | N |
| 11-3 | CH(CH₃)CH₂SO₂CH₃ (S)-isomer | H | H | C—I | CH | CH | C—F | C—CH₃ | N |
| 11-4 | CH(CH₃)CH₂SCH₃ (S)-isomer | H | H | C—Cl | CH | CH | C—Cl | C—CH₃ | N |
| 11-5 | CH(CH₃)CH₂SOCH₃ (S)-isomer | H | H | C—Cl | CH | CH | C—Cl | C—CH₃ | N |
| 11-6 | CH(CH₃)CH₂SO₂CH₃ (S)-isomer | H | H | C—Cl | CH | CH | C—Cl | C—CH₃ | N |
| 11-7 | CH(CH₃)CH₂SCH₃ (S)-isomer | H | H | C—Cl | CH | CH | C—Cl | C—CH₃ | N |
| 11-8 | CH(CH₃)CH₂SOCH₃ (S)-isomer | H | H | C—Cl | CH | CH | C—Cl | C—CH₃ | N |
| 11-9 | CH(CH₃)CH₂SO₂CH₃ (S)-isomer | H | H | C—Cl | CH | CH | C—Cl | C—CH₃ | N |
| 11-10 | CH(CH₃)CH₂SCH₃ (S)-isomer | H | H | C—Cl | CH | CH | C—Cl | C—CH₃ | N |
| 11-11 | CH(CH₃)CH₂SOCH₃ (S)-isomer | H | H | C—Cl | CH | CH | C—Cl | C—CH₃ | N |
| 11-12 | CH(CH₃)CH₂SO₂CH₃ (S)-isomer | H | H | C—Cl | CH | CH | C—Cl | C—CH₃ | N |
| 11-13 | CH(CH₃)CH₂SCH₃ (S)-isomer | H | H | C—Cl | CH | CH | C—Br | C—CH₃ | N |
| 11-14 | CH(CH₃)CH₂SOCH₃ (S)-isomer | H | H | C—Cl | CH | CH | C—Br | C—CH₃ | N |
| 11-15 | CH(CH₃)CH₂SO₂CH₃ (S)-isomer | H | H | C—Cl | CH | CH | C—Br | C—CH₃ | N |
| 11-16 | CH(CH₃)CH₂SCH₃ (S)-isomer | H | H | C—OCH₃ | CH | CH | C—Br | C—CH₃ | N |
| 11-17 | CH(CH₃)CH₂SOCH₃ (S)-isomer | H | H | C—OCH₃ | CH | CH | C—Br | C—CH₃ | N |
| 11-18 | CH(CH₃)CH₂SO₂CH₃ (S)-isomer | H | H | C—OCH₃ | CH | CH | C—Br | C—CH₃ | N |
| 11-19 | CH(CH₃)CH₂SCH₃ (S)-isomer | H | H | C—OCH₃ | CH | CH | C—Br | C—CH₃ | N |
| 11-20 | CH(CH₃)CH₂SOCH₃ (S)-isomer | H | H | C—OCH₃ | CH | CH | C—Br | C—CH₃ | N |
| 11-21 | CH(CH₃)CH₂SCH₃ (S)-isomer | H | H | C—Cl | CH | CH | C—Cl | C—CH₃ | CH |
| 11-22 | CH(CH₃)CH₂SOCH₃ (S)-isomer | H | H | C—Cl | CH | CH | C—Cl | C—CH₃ | CH |
| 11-23 | CH(CH₃)CH₂SO₂CH₃ (S)-isomer | H | H | C—Cl | CH | CH | C—Cl | C—CH₃ | CH |
| 11-24 | CH(CH₃)CH₂SO₂CH₃ (S)-isomer | H | H | C—Cl | CH | CH | C—Cl | C—CH₃ | CH |
| 11-25 | CH(CH₃)CH₂SOCH₃ (S)-isomer | H | H | C—Cl | CH | CH | C—Cl | C—CH₃ | CH |
| 11-26 | CH(CH₃)CH₂SCH₃ (S)-isomer | H | H | C—Cl | CH | CH | C—Cl | C—CH₃ | CH |
| 11-27 | CH(CH₃)CH₂SCH₃ (S)-isomer | H | H | C—Cl | CH | CH | C—Cl | C—CH₃ | CH |
| 11-28 | CH(CH₃)CH₂SOCH₃ (S)-isomer | H | H | C—Cl | CH | CH | C—Cl | C—CH₃ | CH |
| 11-29 | CH(CH₃)CH₂SO₂CH₃ (S)-isomer | H | H | C—Cl | CH | CH | C—Cl | C—CH₃ | CH |
| 11-30 | CH(CH₃)CH₂SCH₃ (S)-isomer | H | H | C—Cl | CH | CH | C—Br | C—CH₃ | CH |
| 11-31 | CH(CH₃)CH₂SOCH₃ (S)-isomer | H | H | C—Cl | CH | CH | C—Br | C—CH₃ | CH |
| 11-32 | CH(CH₃)CH₂SO₂CH₃ (S)-isomer | H | H | C—Cl | CH | CH | C—Br | C—CH₃ | CH |
| 11-33 | CH(CH₃)CH₂SCH₃ (S)-isomer | H | H | C—OCH₃ | CH | CH | C—Br | C—CH₃ | CH |
| 11-34 | CH(CH₃)CH₂SO₂CH₃ (S)-isomer | H | H | C—OCH₃ | CH | CH | C—Br | C—CH₃ | N |
| 11-35 | CH(CH₃)CH₂SCH₃ (S)-isomer | H | H | C—OCH₃ | CH | CH | C—Br | C—CH₃ | CH |
| 11-36 | CH(CH₃)CH₂SCH₃ (S)-isomer | H | H | C—OCH₃ | CH | CH | C—Br | C—CH₃ | CH |
| 11-37 | CH(CH₃)CH₂SCH₃ (S)-isomer | H | H | C—OCH₃ | CH | CH | C—Br | C—CH₃ | CH |
| 11-38 | CH(CH₃)CH₂SCH₃ (S)-isomer | H | H | C—OCH₃ | CH | CH | C—Br | C—CH₃ | CH |
| 11-39 | CH(CH₃)CH₂SCH₃ (S)-isomer | H | H | C—Cl | CH | CH | C—Cl | C—CH₃ | N |
| 11-40 | CH(CH₃)CH₂SCH₃ (S)-isomer | H | H | C—Cl | CH | CH | C—Br | C—CH₃ | N |

| No. | W⁷ | W⁸ | W⁹ | A | r | Q | R⁴ | R⁵ | R⁶ | R⁷ | Physical property |
|---|---|---|---|---|---|---|---|---|---|---|---|
| 11-1 | C—(A)r—Q | CH | CH | CH₂ | 1 | Q8 | CF₃ | H | CF₃ | — | logP (acid) 3.72 |
| 11-2 | C—(A)r—Q | CH | CH | CH₂ | 1 | Q8 | CF₃ | H | CF₃ | — | |
| 11-3 | C—(A)r—Q | CH | CH | CH₂ | 1 | Q8 | CF₃ | H | CF₃ | — | |
| 11-4 | C—(A)r—Q | CH | CH | CH₂ | 1 | Q8 | CF₃ | H | CF₃ | — | logP (acid) 4.14 |
| 11-5 | C—(A)r—Q | CH | CH | CH₂ | 1 | Q8 | CF₃ | H | CF₃ | — | logP (acid) 2.65 |
| 11-6 | C—(A)r—Q | CH | CH | CH₂ | 1 | Q8 | CF₃ | H | CF₃ | — | logP (acid) 3.05 |
| 11-7 | C—(A)r—Q | CH | CH | CH₂ | 1 | Q34 | CF₃ | CF₃ | — | — | |
| 11-8 | C—(A)r—Q | CH | CH | CH₂ | 1 | Q34 | CF₃ | CF₃ | — | — | |
| 11-9 | C—(A)r—Q | CH | CH | CH₂ | 1 | Q34 | CF₃ | CF₃ | — | — | |
| 11-10 | C—(A)r—Q | CH | CH | CH₂ | 1 | Q34 | C₂F₅ | CF₃ | — | — | |
| 11-11 | C—(A)r—Q | CH | CH | CH₂ | 1 | Q34 | C₂F₅ | CF₃ | — | — | |
| 11-12 | C—(A)r—Q | CH | CH | CH₂ | 1 | Q34 | C₂F₅ | CF₃ | — | — | |
| 11-13 | C—(A)r—Q | CH | CH | CH₂ | 1 | Q8 | CF₃ | H | CF₃ | — | logP (acid) 3.89 |
| 11-14 | C—(A)r—Q | CH | CH | CH₂ | 1 | Q8 | CF₃ | H | CF₃ | — | logP (acid) 2.68 |
| 11-15 | C—(A)r—Q | CH | CH | CH₂ | 1 | Q8 | CF₃ | H | CF₃ | — | logP (acid) 3.05 |
| 11-16 | C—(A)r—Q | CH | CH | CH₂ | 1 | Q8 | CF₃ | H | CF₃ | — | logP (acid) 3.63 |
| 11-17 | C—(A)r—Q | CH | CH | CH₂ | 1 | Q8 | CF₃ | H | CF₃ | — | logP (acid) 2.6 |
| 11-18 | C—(A)r—Q | CH | CH | CH₂ | 1 | Q8 | CF₃ | H | CF₃ | — | |
| 11-19 | C—(A)r—Q | CH | CH | CH₂ | 1 | Q8 | C₂F₅ | H | CF₃ | — | logP (acid) 4.02 |
| 11-20 | C—(A)r—Q | CH | CH | CH₂ | 1 | Q8 | C₂F₅ | H | CF₃ | — | logP (acid) 2.90 |
| 11-21 | C—(A)r—Q | CH | CH | CH₂ | 1 | Q8 | CF₃ | H | CF₃ | — | logP (acid) 3.88 |
| 11-22 | C—(A)r—Q | CH | CH | CH₂ | 1 | Q8 | CF₃ | H | CF₃ | — | |
| 11-23 | C—(A)r—Q | CH | CH | CH₂ | 1 | Q8 | CF₃ | H | CF₃ | — | logP (acid) 3.40 |
| 11-24 | C—(A)r—Q | CH | CH | CH₂ | 1 | Q34 | CF₃ | CF₃ | — | — | |
| 11-25 | C—(A)r—Q | CH | CH | CH₂ | 1 | Q34 | CF₃ | CF₃ | — | — | NMR |
| 11-26 | C—(A)r—Q | CH | CH | CH₂ | 1 | Q34 | CF₃ | CF₃ | — | — | logP (acid) 4.25 |
| 11-27 | C—(A)r—Q | CH | CH | CH₂ | 1 | Q34 | C₂F₅ | CF₃ | — | — | logP (acid) 4.51 |
| 11-28 | C—(A)r—Q | CH | CH | CH₂ | 1 | Q34 | C₂F₅ | CF₃ | — | — | logP (neutral) 2.90 |
| 11-29 | C—(A)r—Q | CH | CH | CH₂ | 1 | Q34 | C₂F₅ | CF₃ | — | — | logP (acid) 3.50 |
| 11-30 | C—(A)r—Q | CH | CH | CH₂ | 1 | Q34 | C₂F₅ | CF₃ | — | — | logP (acid) 4.28 |
| 11-31 | C—(A)r—Q | CH | CH | CH₂ | 1 | Q34 | C₂F₅ | CF₃ | — | — | |
| 11-32 | C—(A)r—Q | CH | CH | CH₂ | 1 | Q34 | C₂F₅ | CF₃ | — | — | logP (acid) 3.47 |
| 11-33 | C—(A)r—Q | CH | CH | CH₂ | 1 | Q8 | CF₃ | H | CF₃ | — | |
| 11-34 | C—(A)r—Q | CH | CH | CH₂ | 1 | Q8 | C₂F₅ | H | CF₃ | — | |
| 11-35 | C—(A)r—Q | CH | CH | CH₂ | 1 | Q8 | C₂F₅ | C₂F₅ | H | — | |
| 11-36 | C—(A)r—Q | CH | CH | CH₂ | 1 | Q34 | CF₃ | H | — | — | |
| 11-37 | C—(A)r—Q | CH | CH | CH₂ | 1 | Q34 | CF₃ | CF₃ | — | — | |
| 11-38 | C—(A)r—Q | CH | CH | CH₂ | 1 | Q34 | C₂F₅ | CF₃ | — | — | logP (acid) 4.14 |
| 11-39 | C—(A)r—Q | N | CH | CH₂ | 1 | Q8 | CF₃ | H | CF₃ | — | |
| 11-40 | C—(A)r—Q | N | CH | CH₂ | 1 | Q8 | CF₃ | H | CF₃ | — | |

TABLE 12

| No. | R¹ | R² | R³ | W¹ | W² | W³ | W⁴ | W⁵ |
|---|---|---|---|---|---|---|---|---|
| 12-1 | CH₃ | H | H | CH | C—Cl | CH | C—Cl | C—CH₃ |
| 12-2 | CH(CH₃)₂ | H | H | CH | C—Cl | CH | C—Cl | C—CH₃ |
| 12-3 | CH(CH₃)CH₂SCH₃ (S)-isomer | H | H | CH | C—Cl | CH | C—Cl | C—CH₃ |
| 12-4 | CH₃ | H | H | CH | C—Cl | CH | C—Cl | C—CH₃ |
| 12-5 | CH(CH₃)₂ | H | H | CH | C—Cl | CH | C—Cl | C—CH₃ |
| 12-6 | CH(CH₃)CH₂SCH₃ (S)-isomer | H | H | CH | C—Cl | CH | C—Cl | C—CH₃ |
| 12-7 | CH(CH₃)CH₂SOCH₃ (S)-isomer | H | H | CH | C—Cl | CH | C—Cl | C—CH₃ |
| 12-8 | CH(CH₃)CH₂SO₂CH₃ (S)-isomer | H | H | CH | C—Cl | CH | C—Cl | C—CH₃ |
| 12-9 | CH(CH₃)₂ | H | H | CH | C—Br | CH | C—Br | C—CH₃ |
| 12-10 | CH(CH₃)CH₂SCH₃ (S)-isomer | H | H | CH | C—Br | CH | C—Br | C—CH₃ |
| 12-11 | CH(CH₃)₂ | H | H | CH | C—Br | CH | C—Br | C—CH₃ |
| 12-12 | CH(CH₃)CH₂SCH₃ (S)-isomer | H | H | CH | C—Br | CH | C—Br | C—CH₃ |
| 12-13 | CH(CH₃)₂ | H | H | CH | C—CH₃ | CH | C—CH₃ | C—CH₃ |
| 12-14 | CH(CH₃)CH₂SCH₃ (S)-isomer | H | H | CH | C—CH₃ | CH | C—CH₃ | C—CH₃ |
| 12-15 | CH(CH₃)₂ | H | H | CH | C—CH₃ | CH | C—CH₃ | C—CH₃ |
| 12-16 | CH(CH₃)CH₂SCH₃ (S)-isomer | H | H | CH | C—CH₃ | CH | C—CH₃ | C—CH₃ |
| 12-17 | CH(CH₃)CH₂SCH₃ (S)-isomer | H | H | CH | C—Cl | CH | C—Cl | C—CH₃ |
| 12-18 | CH(CH₃)CH₂SCH₃ (S)-isomer | H | H | CH | C—Br | CH | C—Br | C—CH₃ |

| No. | W⁶ | W⁷ | W⁸ | W⁹ | A | r | Q | R⁴ | R⁵ | R⁶ | R⁷ | Physical property |
|---|---|---|---|---|---|---|---|---|---|---|---|---|
| 12-1 | CH | C—(A)r—Q | CH | CH | CH₂ | 1 | Q8 | CF₃ | H | CF₃ | — | logP (acid) 3.69 |
| 12-2 | CH | C—(A)r—Q | CH | CH | CH₂ | 1 | Q8 | CF₃ | H | CF₃ | — | |
| 12-3 | CH | C—(A)r—Q | CH | CH | CH₂ | 1 | Q8 | CF₃ | H | CF₃ | — | |
| 12-4 | CH | C—(A)r—Q | CH | CH | CH₂ | 1 | Q34 | CF₃ | CF₃ | — | — | logP (acid) 3.39 |
| 12-5 | CH | C—(A)r—Q | CH | CH | CH₂ | 1 | Q34 | CF₃ | CF₃ | — | — | |
| 12-6 | CH | C—(A)r—Q | CH | CH | CH₂ | 1 | Q34 | CF₃ | CF₃ | — | — | NMR |
| 12-7 | CH | C—(A)r—Q | CH | CH | CH₂ | 1 | Q34 | CF₃ | CF₃ | — | — | |
| 12-8 | CH | C—(A)r—Q | CH | CH | CH₂ | 1 | Q34 | CF₃ | CF₃ | — | — | |
| 12-9 | CH | C—(A)r—Q | CH | CH | CH₂ | 1 | Q8 | CF₃ | H | CF₃ | — | |
| 12-10 | CH | C—(A)r—Q | CH | CH | CH₂ | 1 | Q8 | CF₃ | H | CF₃ | — | |
| 12-11 | CH | C—(A)r—Q | CH | CH | CH₂ | 1 | Q34 | CF₃ | CF₃ | — | — | |
| 12-12 | CH | C—(A)r—Q | CH | CH | CH₂ | 1 | Q34 | CF₃ | CF₃ | — | — | |
| 12-13 | CH | C—(A)r—Q | CH | CH | CH₂ | 1 | Q8 | CF₃ | H | CF₃ | — | |
| 12-14 | CH | C—(A)r—Q | CH | CH | CH₂ | 1 | Q8 | CF₃ | H | CF₃ | — | |
| 12-15 | CH | C—(A)r—Q | CH | CH | CH₂ | 1 | Q34 | CF₃ | CF₃ | — | — | |
| 12-16 | CH | C—(A)r—Q | CH | CH | CH₂ | 1 | Q34 | CF₃ | CF₃ | — | — | |
| 12-17 | N | C—(A)r—Q | N | CH | CH₂ | 1 | Q8 | CF₃ | H | CF₃ | — | |
| 12-18 | N | C—(A)r—Q | N | CH | CH₂ | 1 | Q8 | CF₃ | H | CF₃ | — | |

TABLE 13

| No. | R¹ | R² | R³ | W¹ | W² | W³ | W⁴ |
|---|---|---|---|---|---|---|---|
| 13-1 | CH(CH₃)₂ | H | H | CH | CH | CH | CH |
| 13-2 | CH(CH₃)CH₂SCH₃ (S)-isomer | H | H | CH | CH | CH | CH |
| 13-3 | CH(CH₃)CH₂SCH₃ (S)-isomer | H | H | CH | CH | CH | CH |
| 13-4 | CH(CH₃)CH₂SOCH₃ (S)-isomer | H | H | CH | CH | CH | CH |
| 13-5 | CH(CH₃)CH₂SOCH₃ (S)-isomer | H | H | CH | CH | CH | CH |
| 13-6 | CH(CH₃)CH₂SO₂CH₃ (S)-isomer | H | H | CH | CH | CH | CH |
| 13-7 | CH(CH₃)CH₂SCH₃ (S)-isomer | H | H | C—CH₃ | CH | CH | CH |
| 13-8 | CH(CH₃)CH₂SCH₃ (S)-isomer | H | H | C—C₂H₅ | CH | CH | CH |
| 13-9 | CH(CH₃)CH₂SCH₃ (S)-isomer | H | H | C—C₂H₅ | CH | CH | CH |
| 13-10 | CH(CH₃)CH₂SCH₃ (S)-isomer | H | H | C—OCH₃ | CH | CH | CH |
| 13-11 | CH(CH₃)CH₂SCH₃ (S)-isomer | H | H | C—OC₂H₅ | CH | CH | CH |
| 13-12 | CH(CH₃)CH₂SCH₃ (S)-isomer | H | H | C—OCHF₂ | CH | CH | CH |
| 13-13 | CH(CH₃)CH₂SCH₃ (S)-isomer | H | H | C—OCF₃ | CH | CH | CH |
| 13-14 | CH(CH₃)₂ | H | H | C—SO₂CH₃ | CH | CH | CH |
| 13-15 | CH(CH₃)C₂H₅ | H | H | C—SO₂CH₃ | CH | CH | CH |
| 13-16 | CH(CH₃)CH₂OCH₃ | H | H | C—SO₂CH₃ | CH | CH | CH |
| 13-17 | CH₃ | H | H | C—Cl | CH | CH | CH |
| 13-18 | C₃H₇-n | H | H | C—Cl | CH | CH | CH |
| 13-19 | CH(CH₃)₂ | H | H | C—Cl | CH | CH | CH |
| 13-20 | CH(CH₃)C₂H₅ | H | H | C—Cl | CH | CH | CH |
| 13-21 | CH(CH₃)C₃H₇-n | H | H | C—Cl | CH | CH | CH |
| 13-22 | CH(CH₃)C₂H₅ | H | H | CH | CH | CH | C—Cl |
| 13-23 | CH(CH₃)CH₂SCH₃ | H | H | C—Cl | CH | CH | CH |
| 13-24 | CH(CH₃)CH₂SCH₃ (S)-isomer | H | H | C—Cl | CH | CH | CH |
| 13-25 | CH(CH₃)CH₂SCH₃ (S)-isomer | H | H | C—Cl | CH | CH | CH |
| 13-26 | CH(CH₃)CH₂SCH₃ (S)-isomer | H | H | C—Cl | CH | CH | CH |
| 13-27 | CH(CH₃)CH₂SCH₃ (S)-isomer | H | H | C—Cl | CH | CH | CH |
| 13-28 | CH(CH₃)CH₂SCH₃ (S)-isomer | H | H | C—Cl | CH | CH | CH |
| 13-29 | CH(CH₃)CH₂SCH₃ (S)-isomer | H | H | C—Cl | CH | CH | CH |
| 13-30 | CH(CH₃)CH₂SCH₃ (S)-isomer | H | H | C—Cl | CH | CH | CH |
| 13-31 | CH(CH₃)CH₂SCH₃ (S)-isomer | H | H | C—Cl | CH | CH | CH |
| 13-32 | CH(CH₃)CH₂SCH₃ (S)-isomer | H | H | C—SO₂CH₃ | CH | CH | CH |
| 13-33 | CH(CH₃)CH₂SCH₃ (S)-isomer | H | H | C—Cl | CH | CH | CH |
| 13-34 | CH(CH₃)CH₂SCH₃ (S)-isomer | H | H | C—Cl | CH | CH | CH |
| 13-35 | CH(CH₃)CH₂SCH₃ (S)-isomer | H | H | C—Cl | CH | CH | CH |
| 13-36 | CH(CH₃)CH₂SCH₃ (S)-isomer | H | H | C—Cl | CH | CH | CH |
| 13-37 | CH(CH₃)CH₂SCH₃ (S)-isomer | H | H | C—Cl | CH | CH | CH |
| 13-38 | CH(CH₃)CH₂SCH₃ (S)-isomer | H | H | C—Cl | CH | CH | CH |
| 13-39 | CH(CH₃)CH₂SCH₃ (S)-isomer | H | H | C—Cl | CH | CH | CH |
| 13-40 | CH(CH₃)CH₂SCH₃ (S)-isomer | H | H | C—Cl | CH | CH | CH |
| 13-41 | CH(CH₃)CH₂SCH₃ (S)-isomer | H | H | C—Cl | CH | CH | CH |
| 13-42 | CH(CH₃)CH₂SCH₃ (S)-isomer | H | H | C—Cl | CH | CH | CH |
| 13-43 | CH(CH₃)CH₂SCH₃ (S)-isomer | H | H | C—Cl | CH | CH | CH |
| 13-44 | CH(CH₃)CH₂SCH₃ (S)-isomer | H | H | C—Cl | CH | CH | CH |
| 13-45 | CH(CH₃)CH₂SCH₃ (S)-isomer | H | H | C—Cl | CH | CH | CH |
| 13-46 | CH(CH₃)CH₂SCH₃ (S)-isomer | H | H | C—Cl | CH | CH | CH |
| 13-47 | CH(CH₃)CH₂SCH₃ (S)-isomer | H | H | C—Cl | CH | CH | CH |
| 13-48 | CH(CH₃)CH₂SCH₃ (S)-isomer | H | H | C—Cl | CH | CH | CH |
| 13-49 | CH(CH₃)CH₂SCH₃ (S)-isomer | H | H | C—Cl | CH | CH | CH |
| 13-50 | CH(CH₃)CH₂SCH₃ (S)-isomer | H | H | C—Cl | CH | CH | CH |
| 13-51 | CH(CH₃)CH₂SCH₃ (S)-isomer | H | H | C—Cl | CH | CH | CH |
| 13-52 | CH(CH₃)CH₂SCH₃ (S)-isomer | H | H | C—Cl | CH | CH | CH |
| 13-53 | CH(CH₃)CH₂SCH₃ (S)-isomer | H | H | C—Cl | CH | CH | CH |
| 13-54 | CH(CH₃)CH₂SCH₃ (S)-isomer | H | H | C—Cl | CH | CH | CH |
| 13-55 | CH(CH₃)CH₂SCH₃ (S)-isomer | H | H | C—Cl | CH | CH | CH |
| 13-56 | CH(CH₃)CH₂SCH₃ (S)-isomer | H | H | C—Cl | CH | CH | CH |
| 13-57 | CH(CH₃)CH₂SCH₃ (S)-isomer | H | H | C—Cl | CH | CH | CH |
| 13-58 | CH(CH₃)CH₂SOCH₃ (S)-isomer | H | H | C—Cl | CH | CH | CH |
| 13-59 | CH(CH₃)CH₂SOCH₃ (S)-isomer | H | H | C—Cl | CH | CH | CH |
| 13-60 | CH(CH₃)CH₂SOCH₃ (S)-isomer | H | H | C—Cl | CH | CH | CH |
| 13-61 | CH(CH₃)CH₂SOCH₃ (S)-isomer | H | H | C—Cl | CH | CH | CH |
| 13-62 | CH(CH₃)CH₂SOCH₃ (S)-isomer | H | H | C—Cl | CH | CH | CH |
| 13-63 | CH(CH₃)CH₂SOCH₃ (S)-isomer | H | H | C—Cl | CH | CH | CH |
| 13-64 | CH(CH₃)CH₂SOCH₃ (S)-isomer | H | H | C—Cl | CH | CH | CH |
| 13-65 | CH(CH₃)CH₂SOCH₃ (S)-isomer | H | H | C—Cl | CH | CH | CH |
| 13-66 | CH(CH₃)CH₂SOCH₃ (S)-isomer | H | H | C—Cl | CH | CH | CH |
| 13-67 | CH(CH₃)CH₂SOCH₃ (S)-isomer | H | H | C—Cl | CH | CH | CH |
| 13-68 | CH(CH₃)CH₂SOCH₃ (S)-isomer | H | H | C—Cl | CH | CH | CH |
| 13-69 | CH(CH₃)CH₂SOCH₃ (S)-isomer | H | H | C—Cl | CH | CH | CH |
| 13-70 | CH(CH₃)CH₂SOCH₃ (S)-isomer | H | H | C—Cl | CH | CH | CH |
| 13-71 | CH(CH₃)CH₂SOCH₃ (S)-isomer | H | H | C—Cl | CH | CH | CH |
| 13-72 | CH(CH₃)CH₂SOCH₃ (S)-isomer | H | H | C—Cl | CH | CH | CH |
| 13-73 | CH(CH₃)CH₂SOCH₃ (S)-isomer | H | H | C—Cl | CH | CH | CH |
| 13-74 | CH(CH₃)CH₂SOCH₃ (S)-isomer | H | H | C—Cl | CH | CH | CH |
| 13-75 | CH(CH₃)CH₂SOCH₃ (S)-isomer | H | H | C—Cl | CH | CH | CH |
| 13-76 | CH(CH₃)CH₂SO₂CH₃ (S)-isomer | H | H | C—Cl | CH | CH | CH |
| 13-77 | CH(CH₃)CH₂SCH₃ (S)-isomer | H | H | C—Cl | CH | CH | CH |
| 13-78 | CH(CH₃)CH₂SCH₃ (S)-isomer | H | H | C—Cl | CH | CH | CH |
| 13-79 | CH(CH₃)CH₂SOCH₃ (S)-isomer | H | H | C—Cl | CH | CH | CH |
| 13-80 | CH(CH₃)CH₂SO₂CH₃ (S)-isomer | H | H | C—Cl | CH | CH | CH |
| 13-81 | CH(CH₃)CH₂SCH₃ (S)-isomer | H | H | C—SCH₃ | CH | CH | CH |
| 13-82 | CH(CH₃)CH₂SCH₃ (S)-isomer | H | H | C—SCH₃ | CH | CH | CH |
| 13-83 | CH(CH₃)CH₂SCH₃ (S)-isomer | H | H | C—SO₂CH₃ | CH | CH | CH |
| 13-84 | CH(CH₃)CH₂SCH₃ (S)-isomer | H | H | C—SO₂CH₃ | CH | CH | CH |
| 13-85 | CH(CH₃)CH₂SO₂CH₃ (S)-isomer | H | H | C—Cl | CH | CH | CH |
| 13-86 | CH(CH₃)CH₂SO₂CH₃ (S)-isomer | H | H | C—Cl | CH | CH | CH |
| 13-87 | CH(CH₃)CH₂SO₂CH₃ (S)-isomer | H | H | C—Cl | CH | CH | CH |
| 13-88 | CH(CH₃)CH₂SO₂CH₃ (S)-isomer | H | H | C—Cl | CH | CH | CH |
| 13-89 | CH(CH₃)CH₂SO₂CH₃ (S)-isomer | H | H | C—Cl | CH | CH | CH |
| 13-90 | CH(CH₃)CH₂SO₂CH₃ (S)-isomer | H | H | C—Cl | CH | CH | CH |
| 13-91 | CH(CH₃)CH₂SCH₃ (S)-isomer | H | H | C—Cl | CH | CH | CH |
| 13-92 | CH(CH₃)CH₂SCH₃ (S)-isomer | H | H | C—Br | CH | CH | CH |
| 13-93 | CH(CH₃)CH₂SCH₃ (S)-isomer | H | H | C—SCH₃ | CH | CH | CH |
| 13-94 | CH(CH₃)CH₂SCH₃ (S)-isomer | H | H | C—I | CH | CH | CH |
| 13-95 | CH(CH₃)CH₂SCH₃ (S)-isomer | H | H | C—SCH₃ | CH | CH | CH |
| 13-96 | CH(CH₃)CH₂SCH₃ (S)-isomer | H | H | C—SO₂CH₃ | CH | CH | CH |
| 13-97 | CH(CH₃)CH₂SCH₃ (S)-isomer | H | H | C—Cl | CH | CH | CH |
| 13-98 | CH(CH₃)CH₂SCH₃ (S)-isomer | H | H | C—Br | CH | CH | CH |
| 13-99 | CH(CH₃)CH₂SCH₃ (S)-isomer | H | H | C—I | CH | CH | CH |
| 13-100 | CH(CH₃)CH₂SCH₃ (S)-isomer | H | H | C—SCH₃ | CH | CH | CH |
| 13-101 | CH(CH₃)CH₂SCH₃ (S)-isomer | H | H | C—SO₂CH₃ | CH | CH | CH |
| 13-102 | CH(CH₃)CH₂SCH₃ (S)-isomer | H | H | C—Cl | CH | CH | CH |
| 13-103 | CH(CH₃)CH₂SCH₃ (S)-isomer | H | H | C—Cl | CH | CH | CH |
| 13-104 | CH(CH₃)CH₂SCH₃ (S)-isomer | H | H | C—Cl | CH | CH | CH |
| 13-105 | CH(CH₃)CH₂SCH₃ (S)-isomer | H | H | C—SCH₃ | CH | CH | CH |
| 13-106 | CH(CH₃)CH₂SCH₃ (S)-isomer | H | H | C—SCH₃ | CH | CH | CH |
| 13-107 | CH(CH₃)CH₂SO₂CH₃ (S)-isomer | H | H | C—Cl | CH | CH | CH |
| 13-108 | CH(CH₃)CH₂SO₂CH₃ (S)-isomer | H | H | C—Cl | CH | CH | CH |
| 13-109 | CH(CH₃)CH₂SO₂CH₃ (S)-isomer | H | H | C—Cl | CH | CH | CH |
| 13-110 | CH(CH₃)CH₂SO₂CH₃ (S)-isomer | H | H | C—Cl | CH | CH | CH |
| 13-111 | CH(CH₃)CH₂SO₂CH₃ (S)-isomer | H | H | C—Cl | CH | CH | CH |
| 13-112 | CH(CH₃)CH₂SO₂CH₃ (S)-isomer | H | H | C—Cl | CH | CH | CH |
| 13-113 | CH(CH₃)CH₂SO₂CH₃ (S)-isomer | H | H | C—Cl | CH | CH | CH |
| 13-114 | C(CH₃)₂CH₂SCH₃ | H | H | C—Cl | CH | CH | CH |
| 13-115 | CH(CH₃)CH₂OCH₃ | H | H | C—Cl | CH | CH | CH |
| 13-116 | C(CH₃)₂CO₂H | H | H | C—Cl | CH | CH | CH |
| 13-117 | CH(CH₃)CH₂SO₂NH₂ (S)-isomer | H | H | C—Cl | CH | CH | CH |
| 13-118 | CH(CH₃)CH₂SO₂NHCH₃ (S)-isomer | H | H | C—Cl | CH | CH | CH |
| 13-119 | CH(CH₃)CH₂SO₂N(C₂H₅)₂ (S)- | H | H | C—Cl | CH | CH | CH |
| | isomer | | | | | | |
| 13-120 | C(CH₃)₂CO₂CH₂C₆H₅ | H | H | C—Cl | CH | CH | CH |
| 13-121 | C(CH₃)₂CONHCH₂CO₂CH₃ | H | H | C—Cl | CH | CH | CH |
| 13-122 | C(CH₃)₂CONH-cyclopropyl | H | H | C—Cl | CH | CH | CH |
| 13-123 | C(CH₃)₂CONHCH₂C≡CH | H | H | C—Cl | CH | CH | CH |
| 13-124 | CH(CH₃)CH₂SCH₃ (S)-isomer | H | H | C—Cl | CH | CH | CH |
| 13-125 | CH(CH₃)CH₂SCH₃ (S)-isomer | H | H | C—Cl | CH | CH | CH |
| 13-126 | CH(CH₃)CH₂SCH₃ (S)-isomer | H | H | C—Br | CH | CH | CH |
| 13-127 | CH(CH₃)CH₂SCH₃ (S)-isomer | H | H | C—Br | CH | CH | CH |
| 13-128 | CH(CH₃)CH₂SCH₃ (S)-isomer | H | H | C—Br | CH | CH | CH |
| 13-129 | CH(CH₃)CH₂SCH₃ (S)-isomer | H | H | C—Br | CH | CH | CH |
| 13-130 | CH(CH₃)CH₂SCH₃ (S)-isomer | H | H | CH | CH | CH | CH |
| 13-131 | CH(CH₃)CH₂SCH₃ (S)-isomer | H | H | C—Br | CH | CH | CH |
| 13-132 | CH(CH₃)CH₂SCH₃ (S)-isomer | H | H | C—Br | CH | CH | CH |
| 13-133 | CH(CH₃)CH₂SCH₃ (S)-isomer | H | H | C—Br | CH | CH | CH |
| 13-134 | CH(CH₃)CH₂SCH₃ (S)-isomer | H | H | C—Br | CH | CH | CH |
| 13-135 | CH(CH₃)CH₂SCH₃ (S)-isomer | H | H | C—Br | CH | CH | CH |
| 13-136 | CH(CH₃)CH₂SOCH₃ (S)-isomer | H | H | C—Br | CH | CH | CH |
| 13-137 | CH(CH₃)CH₂SOCH₃ (S)-isomer | H | H | C—Br | CH | CH | CH |
| 13-138 | CH(CH₃)CH₂SOCH₃ (S)-isomer | H | H | C—Br | CH | CH | CH |
| 13-139 | CH(CH₃)CH₂SO₂CH₃ (S)-isomer | H | H | C—Br | CH | CH | CH |
| 13-140 | CH(CH₃)CH₂SO₂CH₃ (S)-isomer | H | H | C—Br | CH | CH | CH |
| 13-141 | CH(CH₃)CH₂SO₂CH₃ (S)-isomer | H | H | C—Br | CH | CH | CH |
| 13-142 | C(CH₃)₂CONHCH₂CO₂CH₃ | H | H | C—Br | CH | CH | CH |
| 13-143 | CH(CH₃)₂ | H | H | CH | CH | CH | CH |
| 13-144 | CH(CH₃)CH₂SO₂CH₃ | H | H | C—I | CH | CH | CH |
| 13-145 | CH(CH₃)CH₂SCH₃ (S)-isomer | H | H | C—I | CH | CH | CH |
| 13-146 | CH(CH₃)CH₂SCH₃ (S)-isomer | H | H | C—I | CH | CH | CH |
| 13-147 | CH(CH₃)CH₂SCH₃ (S)-isomer | H | H | C—I | CH | CH | CH |
| 13-148 | CH(CH₃)CH₂SCH₃ (S)-isomer | H | H | C—I | CH | CH | CH |
| 13-149 | CH(CH₃)CH₂SCH₃ (S)-isomer | H | H | C—I | CH | CH | CH |
| 13-150 | CH(CH₃)CH₂SCH₃ (S)-isomer | H | H | C—I | CH | CH | CH |
| 13-151 | CH(CH₃)CH₂SCH₃ (S)-isomer | H | H | C—I | CH | CH | CH |
| 13-152 | CH(CH₃)CH₂SCH₃ (S)-isomer | H | H | C—I | CH | CH | CH |
| 13-153 | CH(CH₃)CH₂SCH₃ (S)-isomer | H | H | C—I | CH | CH | CH |
| 13-154 | CH(CH₃)CH₂SCH₃ (S)-isomer | H | H | C—I | CH | CH | CH |
| 13-155 | CH(CH₃)CH₂SCH₃ (S)-isomer | H | H | C—I | CH | CH | CH |
| 13-156 | CH(CH₃)CH₂SCH₃ (S)-isomer | H | H | C—I | CH | CH | CH |
| 13-157 | CH(CH₃)CH₂SCH₃ (S)-isomer | H | H | C—I | CH | CH | CH |
| 13-158 | CH(CH₃)CH₂SOCH₃ (S)-isomer | H | H | C—I | CH | CH | CH |
| 13-159 | CH(CH₃)CH₂SOCH₃ (S)-isomer | H | H | C—I | CH | CH | CH |
| 13-160 | C(CH₃)₂CH₂SCH₃ | H | H | C—I | CH | CH | CH |
| 13-161 | C(CH₃)₂CH₂SCH₃ | H | H | C—I | CH | CH | CH |
| 13-162 | C(CH₃)₂CH₂SCH₃ | H | H | C—I | CH | CH | CH |
| 13-163 | C(CH₃)₂CH₂SOCH₃ | H | H | C—I | CH | CH | CH |
| 13-164 | C(CH₃)₂CH₂SO₂CH₃ | H | H | C—I | CH | CH | CH |
| 13-165 | C(CH₃)₂CONHCH₂CO₂CH₃ | H | H | C—I | CH | CH | CH |
| 13-166 | CH(CH₃)CH₂SCH₃ (S)-isomer | H | H | C—I | CH | CH | CH |
| 13-167 | CH(CH₃)CH₂SCH₃ (S)-isomer | H | H | C—CF₃ | CH | CH | CH |
| 13-168 | CH(CH₃)CH₂SCH₃ (S)-isomer | H | H | C—C₂F₅ | CH | CH | CH |
| 13-169 | CH(CH₃)CH₂SCH₃ (S)-isomer | H | H | C—CH₂CF₃ | CH | CH | CH |
| 13-170 | CH(CH₃)CH₂SCH₃ (S)-isomer | H | H | C—CH═CH₂ | CH | CH | CH |
| 13-171 | CH(CH₃)CH₂SCH₃ (S)-isomer | H | H | C—C≡CH | CH | CH | CH |
| 13-172 | CH(CH₃)CH₂SCH₃ (S)-isomer | H | H | C—CN | CH | CH | CH |
| 13-173 | CH(CH₃)CH₂SO₂CH₃ (S)-isomer | H | H | C-phenyl | CH | CH | CH |
| 13-174 | CH(CH₃)CH₂SCH₃ (S)-isomer | H | H | CH | C—Cl | CH | CH |
| 13-175 | CH(CH₃)CH₂SCH₃ (S)-isomer | H | H | C—Cl | CH | CH | CH |
| 13-176 | CH(CH₃)CH₂SOCH₃ (S)-isomer | H | H | C—Cl | CH | CH | CH |
| 13-177 | CH(CH₃)CH₂SO₂CH₃ (S)-isomer | H | H | C—Cl | CH | CH | CH |
| 13-178 | CH(CH₃)CH₂SCH₃ (S)-isomer | H | H | C—Br | CH | CH | CH |
| 13-179 | CH(CH₃)CH₂SOCH₃ (S)-isomer | H | H | C—Br | CH | CH | CH |
| 13-180 | CH(CH₃)CH₂SO₂CH₃ (S)-isomer | H | H | C—Br | CH | CH | CH |
| 13-181 | CH(CH₃)CH₂SCH₃ (S)-isomer | H | H | C—I | CH | CH | CH |
| 13-182 | CH(CH₃)CH₂SOCH₃ (S)-isomer | H | H | C—I | CH | CH | CH |
| 13-183 | CH(CH₃)CH₂SO₂CH₃ (S)-isomer | H | H | C—I | CH | CH | CH |
| 13-184 | CH(CH₃)CH₂SCH₃ (S)-isomer | H | H | CH | CH | CH | CH |
| 13-185 | CH(CH₃)CH₂SCH₃ (S)-isomer | H | H | C—Cl | CH | CH | CH |
| 13-186 | CH(CH₃)CH₂SO₂CH₃ (S)-isomer | H | H | C—Cl | CH | CH | CH |
| 13-187 | CH(CH₃)CH₂SO₂CH₃ (S)-isomer | H | H | C—Br | CH | CH | CH |
| 13-188 | CH(CH₃)CH₂SOCH₃ (S)-isomer | H | H | C—Br | CH | CH | CH |
| 13-189 | CH(CH₃)CH₂SO₂CH₃ (S)-isomer | H | H | C—I | CH | CH | CH |
| 13-190 | CH(CH₃)CH₂SOCH₃ (S)-isomer | H | H | C—I | CH | CH | CH |
| 13-191 | CH(CH₃)CH₂N(CH₃)₂ | H | H | C—Cl | CH | CH | CH |
| 13-192 | H | H | H | C—NO₂ | CH | CH | CH |
| 13-193 | CH₃ | H | H | C—Cl | CH | CH | CH |
| 13-194 | CH₃ | H | H | CH | CH | CH | C—Cl |
| 13-195 | CH₃ | H | H | C—NO₂ | CH | CH | CH |
| 13-196 | CH₂C≡CH | H | H | C—Cl | CH | CH | CH |
| 13-197 | CH₂C≡CH | H | H | CH | CH | CH | C—Cl |
| 13-198 | CH₂C≡N | H | H | CH | CH | CH | C—Cl |
| 13-199 | CH(CH₃)CH₂SCH₃ (S)-isomer | H | H | C—Cl | CH | CH | CH |
| 13-200 | CH(CH₃)CH₂SCH₃ (S)-isomer | H | H | C—Cl | CH | CH | CH |
| 13-201 | CH(CH₃)CH₂SCH₃ (S)-isomer | H | H | C—Cl | CH | CH | CH |
| 13-202 | CH(CH₃)CH₂SCH₃ (S)-isomer | H | H | C—Cl | CH | CH | CH |
| 13-203 | CH(CH₃)CH₂SCH₃ (S)-isomer | H | H | C—Cl | CH | CH | CH |
| 13-204 | CH(CH₃)CH₂SCH₃ (S)-isomer | H | H | C—Cl | CH | CH | CH |
| 13-205 | CH(CH₃)CH₂SCH₃ (S)-isomer | H | H | C—Cl | CH | CH | CH |
| 13-206 | CH(CH₃)CH₂SCH₃ (S)-isomer | H | H | C—Cl | CH | CH | CH |
| 13-207 | CH(CH₃)CH₂SCH₃ (S)-isomer | H | H | C—Cl | CH | CH | CH |
| 13-208 | CH(CH₃)CH₂SCH₃ (S)-isomer | H | H | C—Cl | CH | CH | CH |
| 13-209 | CH(CH₃)CH₂SCH₃ (S)-isomer | H | H | C—Cl | CH | CH | CH |
| 13-210 | CH(CH₃)CH₂SCH₃ (S)-isomer | H | H | C—Cl | CH | CH | CH |
| 13-211 | CH(CH₃)CH₂SCH₃ (S)-isomer | H | H | C—Cl | CH | CH | CH |
| 13-212 | CH(CH₃)CH₂SCH₃ (S)-isomer | H | H | C—Cl | CH | CH | CH |
| 13-213 | CH(CH₃)CH₂SCH₃ (S)-isomer | H | H | C—Cl | CH | CH | CH |
| 13-214 | CH(CH₃)CH₂SCH₃ (S)-isomer | H | H | C—Cl | CH | CH | CH |
| 13-215 | CH(CH₃)CH₂SCH₃ (S)-isomer | H | H | C—Cl | CH | CH | CH |
| 13-216 | CH(CH₃)CH₂SCH₃ (S)-isomer | H | H | C—Cl | CH | CH | CH |
| 13-217 | CH(CH₃)CH₂SCH₃ (S)-isomer | H | H | C—Cl | CH | CH | CH |
| 13-218 | CH(CH₃)CH₂SCH₃ (S)-isomer | H | H | C—Cl | CH | CH | CH |
| 13-219 | CH(CH₃)CH₂SCH₃ (S)-isomer | H | H | C—Cl | CH | CH | CH |
| 13-220 | CH(CH₃)CH₂SCH₃ (S)-isomer | H | H | C—Cl | CH | CH | CH |
| 13-221 | CH(CH₃)CH₂SCH₃ (S)-isomer | H | H | C—Cl | CH | CH | CH |
| 13-222 | CH(CH₃)CH₂SCH₃ (S)-isomer | H | H | C—Cl | CH | CH | CH |
| 13-223 | CH(CH₃)CH₂SCH₃ (S)-isomer | H | H | C—Cl | CH | CH | CH |
| 13-224 | CH(CH₃)CH₂SCH₃ (S)-isomer | H | H | C—Cl | CH | CH | CH |
| 13-225 | CH(CH₃)CH₂SCH₃ (S)-isomer | H | H | C—Cl | CH | CH | CH |
| 13-226 | CH(CH₃)CH₂SCH₃ (S)-isomer | H | H | C—Cl | CH | CH | CH |
| 13-227 | CH(CH₃)CH₂SCH₃ (S)-isomer | H | H | C—Cl | CH | CH | CH |
| 13-228 | CH(CH₃)CH₂SCH₃ (S)-isomer | H | H | C—Cl | CH | CH | CH |
| 13-229 | CH(CH₃)CH₂SCH₃ (S)-isomer | H | H | C—Cl | CH | CH | CH |
| 13-230 | CH(CH₃)CH₂SCH₃ (S)-isomer | H | H | C—Cl | CH | CH | CH |
| 13-231 | CH(CH₃)CH₂SCH₃ (S)-isomer | H | H | C—Cl | CH | CH | CH |
| 13-232 | CH(CH₃)CH₂SCH₃ (S)-isomer | H | H | C—Cl | CH | CH | CH |
| 13-233 | CH(CH₃)CH₂SCH₃ (S)-isomer | H | H | C—Cl | CH | CH | CH |
| 13-234 | CH(CH₃)CH₂SCH₃ (S)-isomer | H | H | C—Cl | CH | CH | CH |
| 13-235 | CH(CH₃)CH₂SCH₃ (S)-isomer | H | H | C—Cl | CH | CH | CH |
| 13-236 | CH(CH₃)CH₂SCH₃ (S)-isomer | H | H | C—Cl | CH | CH | CH |
| 13-237 | CH(CH₃)CH₂SCH₃ (S)-isomer | H | H | C—Cl | CH | CH | CH |
| 13-238 | CH(CH₃)CH₂SCH₃ (S)-isomer | H | H | CH | C—Cl | C—Cl | CH |
| 13-239 | CH(CH₃)CH₂SCH₃ (S)-isomer | H | H | C—I | CH | CH | CH |
| 13-240 | CH(CH₃)CH₂SCH₃ (S)-isomer | H | H | C—I | CH | CH | CH |
| 13-241 | CH(CH₃)CH₂SCH₃ (S)-isomer | H | H | C—SCH₃ | CH | CH | CH |
| 13-242 | CH(CH₃)CH₂SCH₃ (S)-isomer | H | H | C—SO₂CH₃ | CH | CH | CH |
| 13-243 | CH(CH₃)CH₂SCH₃ (S)-isomer | H | H | C—I | CH | CH | CH |
| 13-244 | CH(CH₃)CH₂SCH₃ (S)-isomer | H | H | C—NO₂ | CH | CH | CH |
| 13-245 | CH(CH₃)CH₂SCH₃ (S)-isomer | H | H | C—NO₂ | CH | CH | CH |
| 13-246 | CH(CH₃)CH₂SCH₃ (S)-isomer | H | H | C—NO₂ | CH | CH | CH |
| 13-247 | CH(CH₃)CH₂SOCH₃ (S)-isomer | H | H | C—I | CH | CH | CH |
| 13-248 | CH(CH₃)CH₂SO₂CH₃ (S)-isomer | H | H | C—Cl | CH | CH | CH |
| 13-249 | CH(CH₃)CH₂SO₂CH₃ (S)-isomer | H | H | C—Cl | CH | CH | CH |
| 13-250 | CH(CH₃)CH₂SO₂CH₃ (S)-isomer | H | H | C—Cl | CH | CH | CH |
| 13-251 | C(C₂H₅)₂C≡CH | H | H | C—Cl | CH | CH | CH |
| 13-252 | C(CH₃)₂CH(OH)CH₃ | H | H | C—Cl | CH | CH | CH |
| 13-253 | C(CH₃)₂CH₂OH | H | H | C—Cl | CH | CH | CH |
| 13-254 | C(CH₃)₂CH₂NHC(O)C₂H₅ | H | H | C—Cl | CH | CH | CH |
| 13-255 | NHN(CH₃)CO₂C₂H₅ | H | H | C—Cl | CH | CH | CH |
| 13-256 | R1c | H | H | CH | CH | CH | C—Cl |
| 13-257 | R1c | H | H | C—Cl | CH | CH | CH |
| 13-258 | R1d | H | H | CH | CH | CH | C—Cl |
| 13-259 | R1d | H | H | C—Cl | CH | CH | CH |
| 13-260 | R1e | H | H | C—Cl | CH | CH | CH |
| 13-261 | R1f | H | H | C—Cl | CH | CH | CH |
| 13-262 | R1g | H | H | C—Cl | CH | CH | CH |
| 13-263 | R1h | H | H | C—Cl | CH | CH | CH |
| 13-264 | R1h | H | H | CH | CH | CH | C—Cl |
| 13-265 | R1i | H | H | C—Cl | CH | CH | CH |
| 13-266 | R1i | H | H | CH | CH | CH | C—Cl |
| 13-267 | R1j | H | H | C—Cl | CH | CH | CH |
| 13-268 | R1k | H | H | C—Cl | CH | CH | CH |
| 13-269 | R1l | H | H | C—Cl | CH | CH | CH |
| 13-270 | R1m | H | H | C—Cl | CH | CH | CH |
| 13-271 | R1n | H | H | C—Cl | CH | CH | CH |
| 13-272 | R1o | H | H | C—Cl | CH | CH | CH |
| 13-273 | R1p | H | H | C—NO₂ | CH | CH | CH |
| 13-274 | R1o | H | H | CH | CH | CH | C—Cl |
| 13-275 | CH₂CH₂CH₂CHF₂ | H | H | C—Cl | CH | CH | CH |
| 13-276 | R1r | H | H | CH | CH | CH | C—Cl |
| 13-277 | CH(C₂H₅)CH₂CN | H | H | C—Cl | CH | CH | CH |
| 13-278 | R1s | H | H | C—Cl | CH | CH | CH |
| 13-279 | R1s | H | H | CH | CH | CH | C—Cl |
| 13-280 | R1t | H | H | C—Cl | CH | CH | CH |
| 13-281 | R1t | H | H | CH | CH | CH | C—Cl |
| 13-282 | CH(CH₃)CH₂N(CH₃)₂ | H | H | C—Cl | CH | CH | CH |
| 13-283 | CH(CH₃)CH₂N(CH₃)₂ | H | H | CH | CH | CH | C—Cl |
| 13-284 | CH(CH₃)CH₂N(CH₃)₂ | H | H | C—SO₂CH₃ | CH | CH | CH |
| 13-285 | CH(CH₃)CH₂N(C₂H₅)₂ | H | H | CH | CH | CH | C—Cl |
| 13-286 | CH(CH₃)CH₂SCH₃ (S)-isomer | H | H | C—SO₂CH₃ | CH | CH | CH |
| 13-287 | CH(CH₃)CH₂SCH₃ (S)-isomer | H | H | C—SO₂N(CH₃)₂ | CH | CH | CH |
| 13-288 | CH(CH₃)CH₂SOCH₃ (S)-isomer | H | H | C—SO₂N(CH₃)₂ | CH | CH | CH |
| 13-289 | CH(CH₃)CH₂SO₂CH₃ (S)-isomer | H | H | C—SO₂N(CH₃)₂ | CH | CH | CH |
| 13-290 | CH(CH₃)CH₂SCH₃ (S)-isomer | H | H | C—SO₂CH₃ | CH | CH | CH |
| 13-291 | CH(CH₃)CH₂SC₂H5 (S)-isomer | H | H | C—SCH₃ | CH | CH | CH |
| 13-292 | CH(CH₃)CH₂SCH₃ (S)-isomer | H | H | C—SO₂CH₃ | CH | CH | CH |
| 13-293 | CH(CH₃)CH₂SCH₃ (S)-isomer | H | H | C—SCH₃ | CH | CH | CH |
| 13-294 | CH(CH₃)CH₂SOCH₃ (S)-isomer | H | H | C—SOCH₃ | CH | CH | CH |
| 13-295 | CH(CH₃)CH₂SOCH₃ (S)-isomer | H | H | C—SO₂CH₃ | CH | CH | CH |
| 13-296 | CH(CH₃)CH₂SO₂CH₃ (S)-isomer | H | H | C—SO₂CH₃ | CH | CH | CH |
| 13-297 | CH(CH₃)CH₂SOCH₃ (S)-isomer | H | H | C—SOCH₃ | CH | CH | CH |
| 13-298 | CH(CH₃)CH₂SOCH₃ (S)-isomer | H | H | C—SCH₃ | CH | CH | CH |
| 13-299 | CH(CH₃)CH₂SCH₃ (S)-isomer | H | H | C—SCH₃ | CH | CH | CH |
| 13-300 | CH(CH₃)CH₂SCH₃ (S)-isomer | H | H | C—SCH₃ | CH | CH | CH |
| 13-301 | CH(CH₃)CH₂SCH₃ (S)-isomer | H | H | C—SCH₃ | CH | CH | CH |
| 13-302 | CH(CH₃)CH₂SCH₃ (S)-isomer | H | H | C—SCH₃ | CH | CH | CH |
| 13-303 | CH(CH₃)CH₂SCH₃ (S)-isomer | H | H | C—SCH₃ | CH | CH | CH |
| 13-304 | CH(CH₃)CH₂SCH₃ (S)-isomer | H | H | C—SO₂CH₃ | CH | CH | CH |
| 13-305 | CH(CH₃)CH₂SCH₃ (S)-isomer | H | H | C—SCHF₂ | CH | CH | CH |
| 13-306 | CH(CH₃)CH₂SCH₃ (S)-isomer | H | H | C—SCH₃ | CH | CH | CH |
| 13-307 | CH(CH₃)CH₂SCH₃ (S)-isomer | H | H | C—SCH₃ | CH | CH | CH |
| 13-308 | CH(CH₃)CH₂SCH₃ (S)-isomer | H | H | C—SCH₃ | CH | CH | CH |
| 13-309 | CH(CH₃)CH₂SCH₃ (S)-isomer | H | H | C—SO₂CH₃ | CH | CH | CH |
| 13-310 | CH(CH₃)CH₂SCH₃ (S)-isomer | H | H | C—SCH₃ | CH | CH | CH |
| 13-311 | CH(CH₃)CH₂SCH₃ (S)-isomer | H | H | C—SO₂CH₃ | CH | CH | CH |
| 13-312 | CH(CH₃)CH₂SCH₃ (S)-isomer | H | H | CH | CH | CH | C—SCH₂- |
| | | | | | | | cyclopropyl |
| 13-313 | CH(CH₃)CH₂SCH₃ (S)-isomer | H | H | CH | CH | CH | C—SCH₂CH═CH₂ |
| 13-314 | CH(CH₃)CH₂SCH₃ (S)-isomer | H | H | CH | CH | CH | C—SCH₂CF₃ |
| 13-315 | CH(CH₃)CH₂SCH₃ (S)-isomer | H | H | CH | CH | CH | C—SCF₃ |
| 13-316 | CH(CH₃)CH₂SCH₃ (S)-isomer | H | H | C—SCH₂- | CH | CH | CH |
| | | | | cyclopropyl | | | |
| 13-317 | CH(CH₃)CH₂SCH₃ (S)-isomer | H | H | C—SC₃H₇-n | CH | CH | CH |
| 13-318 | CH(CH₃)CH₂SCH₃ (S)-isomer | H | H | C—SCH₂CH═CH₂ | CH | CH | CH |
| 13-319 | CH(CH₃)CH₂SCH₃ (S)-isomer | H | H | C—SCH₂CH(CH₃)₂ | CH | CH | CH |
| 13-320 | CH(CH₃)CH₂SCH₃ (S)-isomer | H | H | C—SCF₃ | CH | CH | CH |
| 13-321 | CH(CH₃)CH₂SCH₃ (S)-isomer | H | H | C—SCH₂CF₃ | CH | CH | CH |
| 13-322 | CH(CH₃)CH₂SCH₃ (S)-isomer | H | H | C—SCH₂Ph | CH | CH | CH |
| 13-323 | CH(CH₃)CH₂SCH₃ (S)-isomer | H | H | C—SOCH₃ | CH | CH | CH |
| 13-324 | CH₂C≡CH | H | H | C—SCH₃ | CH | CH | CH |
| 13-325 | CH(CH₃)CH₂SCH₃ (S)-isomer | H | H | C—SCH₃ | CH | CH | CH |
| 13-326 | CH(CH₃)CH₂SCH₃ (S)-isomer | H | H | C—SCH₃ | CH | CH | CH |
| 13-327 | CH(CH₃)CH₂SCH₃ (S)-isomer | H | H | C—SO₂CH₃ | CH | CH | CH |
| 13-328 | CH(CH₃)CH₂SCH₃ (S)-isomer | H | H | C—SCH₃ | CH | CH | CH |
| 13-329 | CH(CH₃)CH₂SCH₃ (S)-isomer | H | H | C—SCH₃ | CH | CH | CH |
| 13-330 | CH(CH₃)CH₂SCH₃ (S)-isomer | H | H | C—SCH₃ | CH | CH | CH |
| 13-331 | CH(CH₃)CH₂SCH₃ (S)-isomer | H | H | C—SO₂CH₃ | CH | CH | CH |
| 13-332 | CH(CH₃)CH₂SCH₃ (S)-isomer | H | H | C—SCH₃ | CH | CH | CH |
| 13-333 | CH(CH₃)CH₂SCH₃ (S)-isomer | H | H | C—SO₂CH₃ | CH | CH | CH |
| 13-334 | CH(CH₃)CH₂SCH₃ (S)-isomer | H | H | C—SO₂CH₃ | CH | CH | CH |
| 13-335 | CH(CH₃)CH₂SCH₃ (S)-isomer | H | H | C—SCH₃ | CH | CH | CH |
| 13-336 | CH(CH₃)CH₂SCH₃ (S)-isomer | H | H | C—SCH₃ | CH | CH | CH |
| 13-337 | CH(CH₃)CH₂SCH₃ (S)-isomer | H | H | C—SCH₃ | CH | CH | CH |
| 13-338 | CH(CH₃)CH₂SCH₃ (S)-isomer | H | H | C—SCH₃ | CH | CH | CH |
| 13-339 | CH(CH₃)CH₂SCH₃ (S)-isomer | H | H | C—SCH₃ | CH | CH | CH |
| 13-340 | CH(CH₃)CH₂SCH₃ (S)-isomer | H | H | C—SCH₃ | CH | CH | CH |
| 13-341 | CH(CH₃)CH₂SCH₃ (S)-isomer | H | H | C—SO₂CH₃ | CH | CH | CH |
| 13-342 | CH(CH₃)CH₂SCH₃ (S)-isomer | H | H | C—SCH₃ | CH | CH | CH |
| 13-343 | CH(CH₃)CH₂SOCH₃ (S)-isomer | H | H | C—SCH₃ | CH | CH | CH |
| 13-344 | CH(CH₃)CH₂SCH₃ (S)-isomer | H | H | C—SO₂N(CH₃)₂ | CH | CH | CH |
| 13-345 | CH(CH₃)CH₂SCH₃ (S)-isomer | H | H | C—SO₂CH₃ | CH | CH | CH |
| 13-346 | CH(CH₃)CH₂SCH₃ (S)-isomer | H | H | C—SO₂C₂H₅ | CH | CH | CH |
| 13-347 | CH(CH₃)CH₂SCH₃ (S)-isomer | H | H | C—SCH₃ | CH | CH | CH |
| 13-348 | CH(CH₃)CH₂SCH₃ (S)-isomer | H | H | C—SCH₃ | CH | CH | CH |
| 13-349 | CH(CH₃)CH₂SO₂CH₃ (S)-isomer | H | H | C—SO₂N(CH₃)₂ | CH | CH | CH |
| 13-350 | CH(CH₃)CH₂SCH₃ (S)-isomer | H | H | C—SCH₃ | CH | CH | CH |
| 13-351 | CH(CH₃)CH₂SCH₃ (S)-isomer | H | H | C—F | CH | CH | CH |
| 13-352 | CH(CH₃)CH₂SCH₃ (S)-isomer | H | H | C—Cl | CH | CH | CH |
| 13-353 | CH(CH₃)CH₂SCH₃ (S)-isomer | H | H | C—Cl | CH | CH | CH |
| 13-354 | CH(CH₃)CH₂SOCH₃ (S)-isomer | H | H | C—Cl | CH | CH | CH |
| 13-355 | CH(CH₃)CH₂SCH₃ (S)-isomer | H | H | C—SO₂CH₃ | CH | CH | CH |
| 13-356 | CH(CH₃)CH₂SCH₃ (S)-isomer | H | H | C—SO₂CH₃ | CH | CH | CH |
| 13-357 | CH(CH₃)CH₂SCH₃ (S)-isomer | H | H | C—SO₂CH₃ | CH | CH | CH |
| 13-358 | CH(CH₃)CH₂SCH₃ (S)-isomer | H | H | C—SO₂CH₃ | CH | CH | CH |
| 13-359 | CH(CH₃)CH₂SCH₃ (S)-isomer | H | H | C—SO₂CH₃ | CH | CH | CH |
| 13-360 | CH(CH₃)CH₂SCH₃ (S)-isomer | H | H | C—SCH₃ | CH | CH | CH |
| 13-361 | CH(CH₃)CH₂SCH₃ (S)-isomer | H | H | C—Cl | CH | CH | CH |
| 13-362 | CH(CH₃)CH₂SCH₃ (S)-isomer | H | H | C—Cl | CH | CH | CH |
| 13-363 | CH(CH₃)CH₂SOCH₃ (S)-isomer | H | H | C—Cl | CH | CH | CH |
| 13-364 | CH(CH₃)CH₂SO₂CH₃ (S)-isomer | H | H | C—Cl | CH | CH | CH |
| 13-365 | CH(CH₃)CH₂SCH₃ (S)-isomer | H | H | C—Cl | CH | CH | CH |
| 13-366 | CH(CH₃)CH₂SCH₃ (S)-isomer | H | H | C—Cl | CH | CH | CH |
| 13-367 | CH(CH₃)CH₂SCH₃ (S)-isomer | H | H | C—SO₂CH₃ | CH | CH | CH |
| 13-368 | CH(CH₃)CH₂SCH₃ (S)-isomer | H | H | C—SCH₃ | CH | CH | CH |
| 13-369 | CH(CH₃)CH₂SCH₃ (S)-isomer | H | H | C—SO₂CH₃ | CH | CH | CH |
| 13-370 | CH(CH₃)CH₂SCH₃ (S)-isomer | H | H | C—SCH₃ | CH | CH | CH |
| 13-371 | CH(CH₃)CH₂SCH₃ (S)-isomer | H | H | C—SCH₃ | CH | CH | CH |
| 13-372 | CH(CH₃)CH₂SCH₃ (S)-isomer | H | H | C—SCH₃ | CH | CH | CH |
| 13-373 | CH(CH₃)CH₂SCH₃ (S)-isomer | H | H | C—SCH₃ | CH | CH | CH |
| 13-374 | CH₂CH═CH₃ | H | H | C—I | CH | CH | CH |
| 13-375 | N(CH₃)₂ | H | H | C—Cl | CH | CH | CH |
| 13-376 | N(CH₃)₂ | H | H | C—I | CH | CH | CH |
| 13-377 | CH₂Si(CH₃)₃ | H | H | C—I | CH | CH | CH |
| 13-378 | C(CH₃)₂CH₂SCH₃ | H | H | N | CH | CH | CH |
| 13-379 | CH(CH₃)CH₂S-cyclohexyl | H | H | C—I | CH | CH | CH |
| 13-380 | CH(CH₃)CH₂SO-cyclohexyl | H | H | C—I | CH | CH | CH |
| 13-381 | CH(CH₃)CH₂SO₂-cyclohexyl | H | H | C—I | CH | CH | CH |
| 13-382 | C(CH₃)₂CH₂SCH₃ | H | H | CH | CH | CH | N |
| 13-383 | CH(CH₃)CH₂SOCH₃ (S)-isomer | H | H | C—SOCF₃ | CH | CH | CH |
| 13-384 | CH(CH₃)CH₂SO₂CH₃ (S)-isomer | H | H | C—SO₂CF₃ | CH | CH | CH |
| 13-385 | CH(CH₃)CH₂SOCH₃ (S)-isomer | H | H | C—SOCH₂- | CH | CH | CH |
| | | | | cyclopropyl | | | |
| 13-386 | CH(CH₃)CH₂SO₂CH₃ (S)-isomer | H | H | C—SO₂CH₂- | CH | CH | CH |
| | | | | cyclopropyl | | | |
| 13-387 | CH(CH₃)CH₂SCH₃ (S)-isomer | H | H | C—SO₂NHCH₃ | CH | CH | CH |
| 13-388 | CH(CH₃)CH₂SCH₃ (S)-isomer | H | H | C—SO₂NHCH₃ | CH | CH | CH |
| 13-389 | CH(CH₃)₂ | H | H | —OCH₂O— | | CH | CH |
| 13-390 | CH(CH₃)₂ | H | H | —OCF₂O— | | CH | CH |
| 13-391 | CH(CH₃)₂ | H | H | —OCH₂CH₂O— | | CH | CH |
| 13-392 | CH(CH₃)₂ | H | H | —OCF₂CF₂O— | | CH | CH |
| 13-393 | CH(CH₃)CH₂SCH₃ (S)-isomer | H | H | —OCH₂O— | | CH | CH |
| 13-394 | CH(CH₃)CH₂SCH₃ (S)-isomer | H | H | —OCF₂O— | | CH | CH |
| 13-395 | CH(CH₃)CH₂SCH₃ (S)-isomer | H | H | —OCH₂CH₂O— | | CH | CH |
| 13-396 | CH(CH₃)CH₂SCH₃ (S)-isomer | H | H | —OCF₂CF₂O— | | CH | CH |
| 13-397 | CH(CH₃)₂ | H | H | CH | —CH₂CH₂CH₂CH₂— | | CH |
| 13-398 | CH(CH₃)₂ | H | H | CH | —CH═CH—CH═CH— | | CH |
| 13-399 | CH(CH₃)₂ | H | H | CH | —N═CH—CH═N— | | CH |
| 13-400 | CH(CH₃)₂ | H | H | CH | —CH═N—N═CH— | | CH |
| 13-401 | CH(CH₃)₂ | H | H | CH | —O—CH═CH— | | CH |
| 13-402 | CH(CH₃)₂ | H | H | CH | —CH═CH—O— | | CH |
| 13-403 | CH(CH₃)₂ | H | H | CH | —S—CH═CH— | | CH |
| 13-404 | CH(CH₃)₂ | H | H | CH | —CH═CH—S— | | CH |
| 13-405 | CH(CH₃)₂ | H | H | CH | —N(CH₃)—CH═CH— | | CH |
| 13-406 | CH(CH₃)₂ | H | H | CH | —CH═CH—N(CH₃)— | | CH |
| 13-407 | CH(CH₃)₂ | H | H | CH | —N(CH₃)—CH═N— | | CH |
| 13-408 | CH(CH₃)₂ | H | H | CH | —N═CH—N(CH₃)— | | CH |
| 13-409 | CH(CH₃)₂ | H | H | CH | —S—CH═N— | | CH |
| 13-410 | CH(CH₃)₂ | H | H | CH | —N═CH—S— | | CH |
| 13-436 | CH(CH₃)CH₂SO₂CH₃ (S)-isomer | H | H | C—NO₂ | CH | CH | CH |
| 13-437 | CH(CH₃)CH₂SO₂CH₃ (S)-isomer | H | H | C—Cl | CH | CH | CH |
| 13-438 | CH(CH₃)CH₂SCH₃ (S)-isomer | H | H | C—NO₂ | CH | CH | CH |
| 13-439 | CH(CH₃)CH₂SCH₃ (S)-isomer | H | H | C—Cl | CH | CH | CH |
| 13-440 | CH(CH₃)CH₂SCH₃ (S)-isomer | H | H | C—NO₂ | CH | CH | CH |
| 13-441 | CH(CH₃)CH₂SCH₃ (S)-isomer | H | H | C—Cl | CH | CH | CH |
| 13-442 | CH(CH₃)CH₂SCH₃ (S)-isomer | H | H | C—NO₂ | CH | CH | CH |
| 13-443 | CH(CH₃)CH₂SCH₃ (S)-isomer | H | H | C—Cl | CH | CH | CH |
| 13-444 | CH(CH₃)CH₂SCH₃ (S)-isomer | H | H | C—NO₂ | CH | CH | CH |
| 13-445 | CH(CH₃)CH₂SCH₃ (S)-isomer | H | H | C—Cl | CH | CH | CH |
| 13-446 | CH(CH₃)CH₂SCH₃ (S)-isomer | H | H | C—NO₂ | CH | CH | CH |
| 13-447 | CH(CH₃)CH₂SCH₃ (S)-isomer | H | H | C—Cl | CH | CH | CH |
| 13-448 | CH(CH₃)CH₂SCH₃ (S)-isomer | H | H | C—NO₂ | CH | CH | CH |
| 13-449 | CH(CH₃)CH₂SCH₃ (S)-isomer | H | H | C—Cl | CH | CH | CH |
| 13-450 | CH(CH₃)CH₂SCH₃ (S)-isomer | H | H | C—NO₂ | CH | CH | CH |
| 13-451 | CH(CH₃)CH₂SCH₃ (S)-isomer | H | H | C—Cl | CH | CH | CH |
| 13-452 | CH(CH₃)CH₂SCH₃ (S)-isomer | H | H | C—NO₂ | CH | CH | CH |
| 13-453 | CH(CH₃)CH₂SCH₃ (S)-isomer | H | H | C—Cl | CH | CH | CH |
| 13-454 | CH(CH₃)CH₂SCH₃ (S)-isomer | H | H | C—NO₂ | CH | CH | CH |
| 13-455 | CH(CH₃)CH₂SCH₃ (S)-isomer | H | H | C—Cl | CH | CH | CH |
| 13-456 | CH(CH₃)CH₂SCH₃ (S)-isomer | H | H | C—NO₂ | CH | CH | CH |
| 13-457 | CH(CH₃)CH₂SCH₃ (S)-isomer | H | H | C—Cl | CH | CH | CH |
| 13-458 | CH(CH₃)CH₂SCH₃ (S)-isomer | H | H | C—NO₂ | CH | CH | CH |
| 13-459 | CH(CH₃)CH₂SCH₃ (S)-isomer | H | H | C—Cl | CH | CH | CH |
| 13-460 | CH(CH₃)CH₂SCH₃ (S)-isomer | H | H | C—NO₂ | CH | CH | CH |
| 13-461 | CH(CH₃)CH₂SCH₃ (S)-isomer | H | H | C—Cl | CH | CH | CH |
| 13-462 | CH(CH₃)CH₂SCH₃ (S)-isomer | H | H | C—NO₂ | CH | CH | CH |
| 13-463 | CH(CH₃)CH₂SCH₃ (S)-isomer | H | H | C—Cl | CH | CH | CH |
| 13-464 | CH(CH₃)CH₂SCH₃ (S)-isomer | H | H | C—NO₂ | CH | CH | CH |
| 13-465 | CH(CH₃)CH₂SCH₃ (S)-isomer | H | H | C—Cl | CH | CH | CH |
| 13-466 | CH(CH₃)CH₂SCH₃ (S)-isomer | H | H | C—NO₂ | CH | CH | CH |
| 13-467 | CH(CH₃)CH₂SCH₃ (S)-isomer | H | H | C—Cl | CH | CH | CH |
| 13-468 | CH(CH₃)CH₂SCH₃ (S)-isomer | H | H | C—NO₂ | CH | CH | CH |
| 13-469 | CH(CH₃)CH₂SCH₃ (S)-isomer | H | H | C—Cl | CH | CH | CH |
| 13-470 | CH(CH₃)CH₂SCH₃ (S)-isomer | H | H | C—NO₂ | CH | CH | CH |
| 13-471 | CH(CH₃)CH₂SCH₃ (S)-isomer | H | H | C—Cl | CH | CH | CH |
| 13-472 | CH(CH₃)CH₂SCH₃ (S)-isomer | H | H | C—NO₂ | CH | CH | CH |
| 13-473 | CH(CH₃)CH₂SCH₃ (S)-isomer | H | H | C—Cl | CH | CH | CH |
| 13-474 | CH(CH₃)CH₂SCH₃ (S)-isomer | H | H | C—NO₂ | CH | CH | CH |
| 13-475 | CH(CH₃)CH₂SCH₃ (S)-isomer | H | H | C—Cl | CH | CH | CH |
| 13-476 | CH(CH₃)CH₂SCH₃ (S)-isomer | H | H | C—NO₂ | CH | CH | CH |
| 13-477 | CH(CH₃)CH₂SCH₃ (S)-isomer | H | H | C—Cl | CH | CH | CH |
| 13-478 | CH(CH₃)CH₂SCH₃ (S)-isomer | H | H | C—NO₂ | CH | CH | CH |
| 13-479 | CH(CH₃)CH₂SCH₃ (S)-isomer | H | H | C—Cl | CH | CH | CH |
| 13-480 | CH(CH₃)CH₂SCH₃ (S)-isomer | H | H | C—NO₂ | CH | CH | CH |
| 13-481 | CH(CH₃)CH₂SCH₃ (S)-isomer | H | H | C—Cl | CH | CH | CH |
| 13-482 | CH(CH₃)CH₂SCH₃ (S)-isomer | H | H | C—NO₂ | CH | CH | CH |
| 13-483 | CH(CH₃)CH₂SCH₃ (S)-isomer | H | H | C—Cl | CH | CH | CH |
| 13-484 | CH(CH₃)CH₂SCH₃ (S)-isomer | H | H | C—Cl | CH | CH | CH |
| 13-485 | CH(CH₃)CH₂SCH₃ (S)-isomer | H | H | C—Cl | CH | CH | CH |
| 13-486 | CH(CH₃)CH₂SCH₃ (S)-isomer | H | H | C—Cl | CH | CH | CH |
| 13-487 | CH(CH₃)CH₂SCH₃ (S)-isomer | H | H | C—Cl | CH | CH | CH |
| 13-488 | CH(CH₃)CH₂SCH₃ (S)-isomer | H | H | C—Cl | CH | CH | CH |
| 13-489 | CH(CH₃)CH₂SCH₃ (S)-isomer | H | H | C—Cl | CH | CH | CH |
| 13-490 | CH(CH₃)CH₂SCH₃ (S)-isomer | H | H | C—Cl | CH | CH | CH |
| 13-491 | CH(CH₃)CH₂SCH₃ (S)-isomer | H | H | C—Cl | CH | CH | CH |
| 13-492 | CH(CH₃)CH₂SCH₃ (S)-isomer | H | H | C—Cl | CH | CH | CH |
| 13-493 | CH(CH₃)CH₂SCH₃ (S)-isomer | H | H | C—Cl | CH | CH | CH |
| 13-494 | CH(CH₃)CH₂SCH₃ (S)-isomer | H | H | C—Cl | CH | CH | CH |
| 13-495 | CH(CH₃)₂ | H | H | C—Cl | CH | CH | CH |
| 13-496 | CH(CH₃)₂ | H | H | C—Cl | CH | CH | CH |
| 13-497 | CH(CH₃)₂ | H | H | C—Cl | CH | CH | CH |
| 13-498 | CH(CH₃)₂ | H | H | C—Cl | CH | CH | CH |
| 13-499 | CH(CH₃)₂ | H | H | C—Cl | CH | CH | CH |
| 13-500 | CH(CH₃)₂ | H | H | C—Cl | CH | CH | CH |
| 13-501 | CH(CH₃)CH₂SCH₃ (S)-isomer | H | H | C—I | CH | CH | CH |
| 13-502 | CH(CH₃)CH₂SCH₃ (S)-isomer | H | H | C—Cl | CH | CH | CH |
| 13-503 | CH(CH₃)CH₂SCH₃ (S)-isomer | H | H | C—SO₂CH₃ | CH | CH | CH |
| 13-504 | CH(CH₃)CH₂SCH₃ (S)-isomer | H | H | C—SO₂CH₃ | CH | CH | CH |
| 13-505 | CH(CH₃)CH₂SCH₃ (S)-isomer | H | H | C—SO₂CH₃ | CH | CH | CH |
| 13-506 | C(CH₃)₂CONHCH₂CH═CH₂ | H | H | C—Cl | CH | CH | CH |
| 13-507 | CH(CH3)CH2SCH3 (S)-isomer | H | H | C—I | CH | CH | CH |

| No. | W⁵ | W⁶ | W⁷ | W⁸ | W⁹ | A | r | Q | R⁴ |
|---|---|---|---|---|---|---|---|---|---|
| 13-1 | C—CH₃ | CH | C—(A)r—Q | CH | CH | CH₂ | 1 | Q8 | CF₃ |
| 13-2 | C—CH₃ | CH | C—(A)r—Q | CH | CH | CH₂ | 1 | Q8 | CF₃ |
| 13-3 | C—CH₃ | CH | C—(A)r—Q | CH | CH | CH₂ | 1 | Q8 | C₂F₅ |
| 13-4 | C—CH₃ | CH | C—(A)r—Q | CH | CH | CH₂ | 1 | Q8 | CF₃ |
| 13-5 | C—CH₃ | CH | C—(A)r—Q | CH | CH | CH₂ | 1 | Q8 | C₂F₅ |
| 13-6 | C—CH₃ | CH | C—(A)r—Q | CH | CH | CH₂ | 1 | Q8 | CF₃ |
| 13-7 | C—CH₃ | CH | C—(A)r—Q | CH | CH | CH₂ | 1 | Q8 | CF₃ |
| 13-8 | C—CH₃ | CH | C—(A)r—Q | CH | CH | CH₂ | 1 | Q8 | CF₃ |
| 13-9 | C—CH₃ | CH | C—(A)r—Q | CH | CH | CH₂ | 1 | Q8 | C₂F₅ |
| 13-10 | C—CH₃ | CH | C—(A)r—Q | CH | CH | CH₂ | 1 | Q8 | CF₃ |
| 13-11 | C—CH₃ | CH | C—(A)r—Q | CH | CH | CH₂ | 1 | Q8 | CF₃ |
| 13-12 | C—CH₃ | CH | C—(A)r—Q | CH | CH | CH₂ | 1 | Q8 | CF₃ |
| 13-13 | C—CH₃ | CH | C—(A)r—Q | CH | CH | CH₂ | 1 | Q8 | CF₃ |
| 13-14 | C—CH₃ | CH | C—(A)r—Q | CH | CH | CH₂ | 1 | Q8 | CF₃ |
| 13-15 | C—CH₃ | CH | C—(A)r—Q | CH | CH | CH₂ | 1 | Q8 | CF₃ |
| 13-16 | C—CH₃ | CH | C—(A)r—Q | CH | CH | CH₂ | 1 | Q8 | CF₃ |
| 13-17 | C—CH₃ | CH | C—(A)r—Q | CH | CH | CH₂ | 1 | Q8 | C₂F₅ |
| 13-18 | C—CH₃ | CH | C—(A)r—Q | CH | CH | CH₂ | 1 | Q8 | CF₃ |
| 13-19 | C—CH₃ | CH | C—(A)r—Q | CH | CH | CH₂ | 1 | Q8 | C₂F₅ |
| 13-20 | C—CH₃ | CH | C—(A)r—Q | CH | CH | CH₂ | 1 | Q8 | CF₃ |
| 13-21 | C—CH₃ | CH | C—(A)r—Q | CH | CH | CH₂ | 1 | Q8 | CF₃ |
| 13-22 | C—CH₃ | CH | C—(A)r—Q | CH | CH | CH₂ | 1 | Q8 | CF₃ |
| 13-23 | C—CH₃ | CH | C—(A)r—Q | CH | CH | CH(CN) | 1 | Q8 | CF₃ |
| 13-24 | C—CH₃ | CH | C—(A)r—Q | CH | CH | CH₂ | 1 | Q8 | I |
| 13-25 | C—CH₃ | CH | C—(A)r—Q | CH | CH | — | 0 | Q8 | H |
| 13-26 | C—CH₃ | CH | C—(A)r—Q | CH | CH | CH₂ | 1 | Q8 | H |
| 13-27 | C—CH₃ | N | C—(A)r—Q | CH | CH | CH₂ | 1 | Q8 | CHF₂ |
| 13-28 | C—CH₃ | N | C—(A)r—Q | CH | CH | CH₂ | 1 | Q8 | CHF₂ |
| 13-29 | C—CH₃ | CH | C—(A)r—Q | CH | CH | CH₂ | 1 | Q8 | [3,5- |
| | | | | | | | | | bis(trifluoromethyl)- |
| | | | | | | | | | 1H-pyrazol- |
| | | | | | | | | | 1-yl]methyl |
| 13-30 | C—CH₃ | CH | C—(A)r—Q | CH | CH | CH(C₂H₅) | 1 | Q8 | CF₃ |
| 13-31 | C—CH₃ | CH | C—(A)r—Q | CH | CH | CH₂ | 1 | Q8 | CF₃ |
| 13-32 | C—CH₃ | CH | C—(A)r—Q | CH | CH | CH₂ | 1 | Q8 | F |
| 13-33 | C—CH₃ | CH | C—(A)r—Q | CH | CH | CH(CO₂C₂H₅) | 1 | Q8 | CF₃ |
| 13-34 | C—Br | CH | C—(A)r—Q | CH | CH | CH₂ | 1 | Q8 | CF₃ |
| 13-35 | C—CH₃ | CH | C—(A)r—Q | CH | CH | CH₂ | 1 | Q8 | CF₃ |
| 13-36 | C—CH₃ | CH | C—(A)r—Q | CH | CH | CH₂ | 1 | Q8 | CF₃ |
| 13-37 | C—CH₃ | CH | C—(A)r—Q | CH | CH | CH₂ | 1 | Q8 | CF₃ |
| 13-38 | C—CH₃ | CH | C—(A)r—Q | CH | CH | CH(CH₃) | 1 | Q8 | C₃F₇-n |
| 13-39 | C—CH₃ | N | C—(A)r—Q | CH | CH | CH₂ | 1 | Q8 | C₃F₇-n |
| 13-40 | C—CH₃ | CH | C—(A)r—Q | CH | CH | CH(CH₃) | 1 | Q8 | C₂F₅ |
| 13-41 | C—CH₃ | CH | C—(A)r—Q | CH | CH | CH₂ | 1 | Q8 | C₂F₅ |
| 13-42 | C—CH₃ | CH | C—(A)r—Q | CH | CH | CH₂ | 1 | Q8 | C₂F₅ |
| 13-43 | C—CH₃ | CH | C—(A)r—Q | CH | CH | CH₂ | 1 | Q8 | C₂F₅ |
| 13-44 | C—CH₃ | CH | C—(A)r—Q | CH | CH | CH₂ | 1 | Q8 | C₂F₅ |
| 13-45 | C—I | CH | C—(A)r—Q | CH | CH | CH₂ | 1 | Q8 | C₂F₅ |
| 13-46 | C—F | CH | C—(A)r—Q | CH | CH | CH₂ | 1 | Q8 | C₂F₅ |
| 13-47 | C—CH₃ | CH | C—(A)r—Q | CH | CH | CH₂ | 1 | Q8 | C₂F₅ |
| 13-48 | C—CH₃ | CH | C—(A)r—Q | CH | CH | CH₂ | 1 | Q8 | C₂F₅ |
| 13-49 | C—CH₃ | CH | C—(A)r—Q | CH | CH | CH₂ | 1 | Q8 | C₂F₅ |
| 13-50 | C—CH₃ | N | C—(A)r—Q | CH | CH | CH₂ | 1 | Q8 | C₂F₅ |
| 13-51 | C—CH₃ | CH | C—(A)r—Q | CH | CH | CH₂ | 1 | Q8 | 4- |
| | | | | | | | | | (trifluoromethyl)phenyl |
| 13-52 | C—CH₃ | CH | C—(A)r—Q | CH | CH | CH₂ | 1 | Q8 | 4- |
| | | | | | | | | | (trifluoromethyl)phenyl |
| 13-53 | C—CH₃ | CH | C—(A)r—Q | CH | CH | CH₂ | 1 | Q8 | 4- |
| | | | | | | | | | (trifluoromethyl)phenyl |
| 13-54 | C—CH₃ | CH | C—(A)r—Q | CH | CH | CH₂ | 1 | Q8 | 3- |
| | | | | | | | | | (trifluoromethyl)phenyl |
| 13-55 | C—CH₃ | CH | C—(A)r—Q | CH | CH | CH₂ | 1 | Q8 | 3- |
| | | | | | | | | | (trifluoromethyl)phenyl |
| 13-56 | C—CH₃ | CH | C—(A)r—Q | CH | CH | CH₂ | 1 | Q8 | 3- |
| | | | | | | | | | (trifluoromethyl)phenyl |
| 13-57 | C—CH₃ | CH | C—(A)r—Q | CH | CH | CH₂ | 1 | Q8 | 3- |
| | | | | | | | | | (trifluoromethyl)phenyl |
| 13-58 | C—CH₃ | N | C—(A)r—Q | CH | CH | CH₂ | 1 | Q8 | CHF₂ |
| 13-59 | C—CH₃ | CH | C—(A)r—Q | CH | CH | CH(C₂H₅) | 1 | Q8 | CF₃ |
| 13-60 | C—CH₃ | CH | C—(A)r—Q | CH | CH | CH₂ | 1 | Q8 | CF₃ |
| 13-61 | C—CH₃ | CH | C—(A)r—Q | CH | CH | CH₂ | 1 | Q8 | CF₃ |
| 13-62 | C—CH₃ | CH | C—(A)r—Q | CH | CH | CH(CH₃) | 1 | Q8 | C₃F₇-n |
| 13-63 | C—CH₃ | CH | C—(A)r—Q | CH | CH | CH(CH₃) | 1 | Q8 | C₂F₅ |
| 13-64 | C—CH₃ | CH | C—(A)r—Q | CH | CH | CH₂ | 1 | Q8 | C₂F₅ |
| 13-65 | C—CH₃ | CH | C—(A)r—Q | CH | CH | CH₂ | 1 | Q8 | C₂F₅ |
| 13-66 | C—CH₃ | CH | C—(A)r—Q | CH | CH | CH₂ | 1 | Q8 | C₂F₅ |
| 13-67 | C—CH₃ | CH | C—(A)r—Q | CH | CH | CH₂ | 1 | Q8 | C₂F₅ |
| 13-68 | C—CH₃ | CH | C—(A)r—Q | CH | CH | CH₂ | 1 | Q8 | C₂F₅ |
| 13-69 | C—CH₃ | N | C—(A)r—Q | CH | CH | CH₂ | 1 | Q8 | C₂F₅ |
| 13-70 | C—CH₃ | N | C—(A)r—Q | CH | CH | CH₂ | 1 | Q8 | C₂F₅ |
| 13-71 | C—CH₃ | CH | C—(A)r—Q | CH | CH | CH₂ | 1 | Q8 | 4- |
| | | | | | | | | | (trifluoromethyl)phenyl |
| 13-72 | C—CH₃ | CH | C—(A)r—Q | CH | CH | CH₂ | 1 | Q8 | 4- |
| | | | | | | | | | (trifluoromethyl)phenyl |
| 13-73 | C—CH₃ | CH | C—(A)r—Q | CH | CH | CH₂ | 1 | Q8 | 3- |
| | | | | | | | | | (trifluoromethyl)phenyl |
| 13-74 | C—CH₃ | CH | C—(A)r—Q | CH | CH | CH₂ | 1 | Q8 | 3- |
| | | | | | | | | | (trifluoromethyl)phenyl |
| 13-75 | C—CH₃ | CH | C—(A)r—Q | CH | CH | CH₂ | 1 | Q8 | 3- |
| | | | | | | | | | (trifluoromethyl)phenyl |
| 13-76 | C—CH₃ | N | C—(A)r—Q | CH | CH | CH₂ | 1 | Q8 | CHF₂ |
| 13-77 | C—CH₃ | CH | C—(A)r—Q | CH | CH | CH₂ | 1 | Q8 | CF₃ |
| 13-78 | C—CH₃ | CH | C—(A)r—Q | CH | CH | CH₂ | 1 | Q8 | C₂F₅ |
| 13-79 | C—CH₃ | CH | C—(A)r—Q | CH | CH | CH₂ | 1 | Q8 | CF₃ |
| 13-80 | C—CH₃ | CH | C—(A)r—Q | CH | CH | CH₂ | 1 | Q8 | CF₃ |
| 13-81 | C—CH₃ | CH | C—(A)r—Q | CH | CH | CH₂ | 1 | Q8 | CF₃ |
| 13-82 | C—CH₃ | CH | C—(A)r—Q | CH | CH | CH₂ | 1 | Q8 | C₂F₅ |
| 13-83 | C—CH₃ | CH | C—(A)r—Q | CH | CH | CH₂ | 1 | Q8 | CF₃ |
| 13-84 | C—CH₃ | CH | C—(A)r—Q | CH | CH | CH₂ | 1 | Q8 | C₂F₅ |
| 13-85 | C—CH₃ | CH | C—(A)r—Q | CH | CH | CH₂ | 1 | Q8 | CF₃ |
| 13-86 | C—CH₃ | CH | C—(A)r—Q | CH | CH | CH₂ | 1 | Q8 | CF₃ |
| 13-87 | C—CH₃ | CH | C—(A)r—Q | CH | CH | CH(CH₃) | 1 | Q8 | C₃F₇-n |
| 13-88 | C—CH₃ | CH | C—(A)r—Q | CH | CH | CH(CH₃) | 1 | Q8 | C₂F₅ |
| 13-89 | C—CH₃ | CH | C—(A)r—Q | CH | CH | CH₂ | 1 | Q8 | C₂F₅ |
| 13-90 | C—CH₃ | CH | C—(A)r—Q | CH | CH | CH₂ | 1 | Q8 | C₂F₅ |
| 13-91 | C—CH₃ | CH | C—(A)r—Q | CH | CH | CH₂ | 1 | Q8 | CF₃ |
| 13-92 | C—CH₃ | CH | C—(A)r—Q | CH | CH | CH₂ | 1 | Q8 | CF₃ |
| 13-93 | C—CH₃ | CH | C—(A)r—Q | CH | CH | CH₂ | 1 | Q8 | CF₃ |
| 13-94 | C—CH₃ | CH | C—(A)r—Q | CH | CH | CH₂ | 1 | Q8 | CF₃ |
| 13-95 | C—CH₃ | CH | C—(A)r—Q | CH | CH | CH₂ | 1 | Q8 | CF₃ |
| 13-96 | C—CH₃ | CH | C—(A)r—Q | CH | CH | CH₂ | 1 | Q8 | CF₃ |
| 13-97 | C—CH₃ | CH | C—(A)r—Q | CH | CH | CH₂ | 1 | Q8 | C₂F₅ |
| 13-98 | C—CH₃ | CH | C—(A)r—Q | CH | CH | CH₂ | 1 | Q8 | C₂F₅ |
| 13-99 | C—CH₃ | CH | C—(A)r—Q | CH | CH | CH₂ | 1 | Q8 | C₂F₅ |
| 13-100 | C—CH₃ | CH | C—(A)r—Q | CH | CH | CH₂ | 1 | Q8 | C₂F₅ |
| 13-101 | C—CH₃ | CH | C—(A)r—Q | CH | CH | CH₂ | 1 | Q8 | C₂F₅ |
| 13-102 | C—CH₃ | CH | C—(A)r—Q | CH | CH | CH₂ | 1 | Q8 | C₃F₇-n |
| 13-103 | C—CH₃ | CH | C—(A)r—Q | CH | CH | CH₂ | 1 | Q8 | C₄F₉-n |
| 13-104 | C—CH₃ | CH | C—(A)r—Q | CH | CH | CH₂ | 1 | Q8 | C₇F₁₅-n |
| 13-105 | C—CH₃ | CH | C—(A)r—Q | CH | CH | CH₂ | 1 | Q8 | C₃F₇-n |
| 13-106 | C—CH₃ | CH | C—(A)r—Q | CH | CH | CH₂ | 1 | Q8 | C₄F₉-n |
| 13-107 | C—CH₃ | CH | C—(A)r—Q | CH | CH | CH₂ | 1 | Q8 | C₂F₅ |
| 13-108 | C—CH₃ | CH | C—(A)r—Q | CH | CH | CH₂ | 1 | Q8 | C₂F₅ |
| 13-109 | C—CH₃ | CH | C—(A)r—Q | CH | CH | CH₂ | 1 | Q8 | C₂F₅ |
| 13-110 | C—CH₃ | N | C—(A)r—Q | CH | CH | CH₂ | 1 | Q8 | C₂F₅ |
| 13-111 | C—CH₃ | CH | C—(A)r—Q | CH | CH | CH₂ | 1 | Q8 | 4- |
| | | | | | | | | | (trifluoromethyl)phenyl |
| 13-112 | C—CH₃ | CH | C—(A)r—Q | CH | CH | CH₂ | 1 | Q8 | 3- |
| | | | | | | | | | (trifluoromethyl)phenyl |
| 13-113 | C—CH₃ | CH | C—(A)r—Q | CH | CH | CH₂ | 1 | Q8 | 3- |
| | | | | | | | | | (trifluoromethyl)phenyl |
| 13-114 | C—CH₃ | CH | C—(A)r—Q | CH | CH | CH₂ | 1 | Q8 | C₂F₅ |
| 13-115 | C—CH₃ | CH | C—(A)r—Q | CH | CH | CH₂ | 1 | Q8 | CF₃ |
| 13-116 | C—CH₃ | CH | C—(A)r—Q | CH | CH | CH₂ | 1 | Q8 | CF₃ |
| 13-117 | C—CH₃ | CH | C—(A)r—Q | CH | CH | CH₂ | 1 | Q8 | CF₃ |
| 13-118 | C—CH₃ | CH | C—(A)r—Q | CH | CH | CH₂ | 1 | Q8 | CF₃ |
| 13-119 | C—CH₃ | CH | C—(A)r—Q | CH | CH | CH₂ | 1 | Q8 | CF₃ |
| 13-120 | C—CH₃ | CH | C—(A)r—Q | CH | CH | CH₂ | 1 | Q8 | CF₃ |
| 13-121 | C—CH₃ | CH | C—(A)r—Q | CH | CH | CH₂ | 1 | Q8 | CF₃ |
| 13-122 | C—CH₃ | CH | C—(A)r—Q | CH | CH | CH₂ | 1 | Q8 | CF₃ |
| 13-123 | C—CH₃ | CH | C—(A)r—Q | CH | CH | CH₂ | 1 | Q8 | CF₃ |
| 13-124 | C—CH₃ | CH | C—(A)r—Q | CH | CH | CO | 1 | Q25 | C₂F₅ |
| 13-125 | C—CH₃ | CH | C—(A)r—Q | CH | CH | C═NOCH₃ | 1 | Q25 | C₂F₅ |
| 13-126 | C—CH₃ | N | C—(A)r—Q | CH | CH | CH₂ | 1 | Q8 | CHF₂ |
| 13-127 | C—CH₃ | CH | C—(A)r—Q | CH | CH | CH₂ | 1 | Q8 | CF₃ |
| 13-128 | C—CH₃ | N | C—(A)r—Q | CH | CH | CH₂ | 1 | Q8 | C₃F₇-n |
| 13-129 | C—CH₃ | CH | C—(A)r—Q | CH | CH | CH₂ | 1 | Q8 | C₂F₅ |
| 13-130 | C—Cl | CH | C—(A)r—Q | CH | CH | CH₂ | 1 | Q8 | CF₃ |
| 13-131 | C—Cl | CH | C—(A)r—Q | CH | CH | CH₂ | 1 | Q8 | C₂F₅ |
| 13-132 | C—CH₃ | CH | C—(A)r—Q | CH | CH | CH₂ | 1 | Q8 | C₂F₅ |
| 13-133 | C—CH₃ | CH | C—(A)r—Q | CH | CH | CH₂ | 1 | Q8 | C₂F₅ |
| 13-134 | C—CH₃ | N | C—(A)r—Q | CH | CH | CH₂ | 1 | Q8 | C₂F₅ |
| 13-135 | C—CH₃ | N | C—(A)r—Q | CH | CH | CH₂ | 1 | Q8 | C₂F₅ |
| 13-136 | C—CH₃ | N | C—(A)r—Q | CH | CH | CH₂ | 1 | Q8 | C₃F₇-n |
| 13-137 | C—CH₃ | CH | C—(A)r—Q | CH | CH | CH₂ | 1 | Q8 | C₂F₅ |
| 13-138 | C—CH₃ | N | C—(A)r—Q | CH | CH | CH₂ | 1 | Q8 | C₂F₅ |
| 13-139 | C—CH₃ | N | C—(A)r—Q | CH | CH | CH₂ | 1 | Q8 | C₃F₇-n |
| 13-140 | C—CH₃ | CH | C—(A)r—Q | CH | CH | CH₂ | 1 | Q8 | C₂F₅ |
| 13-141 | C—CH₃ | N | C—(A)r—Q | CH | CH | CH₂ | 1 | Q8 | C₂F₅ |
| 13-142 | C—CH₃ | CH | C—(A)r—Q | CH | CH | CH₂ | 1 | Q8 | CF₃ |
| 13-143 | C—I | CH | C—(A)r—Q | CH | CH | CH₂ | 1 | Q8 | CF₃ |
| 13-144 | C—CH₃ | CH | C—(A)r—Q | CH | CH | OCH₂CH₂ | 1 | Q8 | CF₃ |
| 13-145 | C—CH₃ | N | C—(A)r—Q | CH | CH | CH₂ | 1 | Q8 | CHF₂ |
| 13-146 | C—CH₃ | CH | C—(A)r—Q | CH | CH | CH₂ | 1 | Q8 | CF₃ |
| 13-147 | C—CH₃ | CH | C—(A)r—Q | CH | CH | CH₂ | 1 | Q8 | CF₃ |
| 13-148 | C—CH₃ | CH | C—(A)r—Q | CH | CH | CH(C₂H₅) | 1 | Q8 | CF₃ |
| 13-149 | C—Cl | CH | C—(A)r—Q | CH | CH | CH₂ | 1 | Q8 | CF₃ |
| 13-150 | C—Br | CH | C—(A)r—Q | CH | CH | CH₂ | 1 | Q8 | CF₃ |
| 13-151 | C—CH₃ | CH | C—(A)r—Q | CH | CH | CH₂ | 2 | Q8 | CF₃ |
| 13-152 | C—CH₃ | CH | C—(A)r—Q | CH | CH | OCH₂CH₂ | 1 | Q8 | CF₃ |
| 13-153 | C—CH₃ | CH | C—(A)r—Q | CH | CH | CH₂ | 1 | Q8 | C₂F₅ |
| 13-154 | CH | CH | C—(A)r—Q | CH | CH | CH₂ | 1 | Q8 | C₂F₅ |
| 13-155 | C—CH₃ | CH | C—(A)r—Q | CH | CH | CH₂ | 1 | Q8 | C₂F₅ |
| 13-156 | C—CH₃ | N | C—(A)r—Q | CH | CH | CH₂ | 1 | Q8 | C₂F₅ |
| 13-157 | C—CH₃ | N | C—(A)r—Q | CH | CH | CH₂ | 1 | Q8 | C₂F₅ |
| 13-158 | C—CH₃ | CH | C—(A)r—Q | CH | CH | CH(C₂H₅) | 1 | Q8 | CF₃ |
| 13-159 | C—CH₃ | N | C—(A)r—Q | CH | CH | CH₂ | 1 | Q8 | C₂F₅ |
| 13-160 | CH | CH | C—(A)r—Q | CH | CH | CH₂ | 1 | Q8 | CF₃ |
| 13-161 | C—CH₃ | CH | C—(A)r—Q | CH | CH | CH₂ | 2 | Q8 | CF₃ |
| 13-162 | C—CH₃ | CH | C—(A)r—Q | CH | CH | CH₂ | 1 | Q8 | 3,5- |
| | | | | | | | | | bis(trifluoromethyl)phenyl |
| 13-163 | CH | CH | C—(A)r—Q | CH | CH | CH₂ | 1 | Q8 | CF₃ |
| 13-164 | CH | CH | C—(A)r—Q | CH | CH | CH₂ | 1 | Q8 | CF₃ |
| 13-165 | C—CH₃ | CH | C—(A)r—Q | CH | CH | CH₂ | 1 | Q8 | CF₃ |
| 13-166 | C—CH₃ | CH | C—(A)r—Q | CH | CH | CH₂ | 1 | Q1 | H |
| 13-167 | C—CH₃ | CH | C—(A)r—Q | CH | CH | CH₂ | 1 | Q8 | CF₃ |
| 13-168 | C—CH₃ | CH | C—(A)r—Q | CH | CH | CH₂ | 1 | Q8 | CF₃ |
| 13-169 | C—CH₃ | CH | C—(A)r—Q | CH | CH | CH₂ | 1 | Q8 | CF₃ |
| 13-170 | C—CH₃ | CH | C—(A)r—Q | CH | CH | CH₂ | 1 | Q8 | CF₃ |
| 13-171 | C—CH₃ | CH | C—(A)r—Q | CH | CH | CH₂ | 1 | Q8 | CF₃ |
| 13-172 | C—CH₃ | CH | C—(A)r—Q | CH | CH | CH₂ | 1 | Q8 | CF₃ |
| 13-173 | C—CH₃ | CH | C—(A)r—Q | CH | CH | CH₂ | 1 | Q8 | CF₃ |
| 13-174 | C—CH₃ | CH | C—(A)r—Q | CH | CH | CH₂ | 1 | Q8 | CF₃ |
| 13-175 | C—CH₃ | CH | C—(A)r—Q | N | CH | CH₂ | 1 | Q8 | CF₃ |
| 13-176 | C—CH₃ | CH | C—(A)r—Q | N | CH | CH₂ | 1 | Q8 | CF₃ |
| 13-177 | C—CH₃ | CH | C—(A)r—Q | N | CH | CH₂ | 1 | Q8 | CF₃ |
| 13-178 | C—CH₃ | CH | C—(A)r—Q | N | CH | CH₂ | 1 | Q8 | CF₃ |
| 13-179 | C—CH₃ | CH | C—(A)r—Q | N | CH | CH₂ | 1 | Q8 | CF₃ |
| 13-180 | C—CH₃ | CH | C—(A)r—Q | N | CH | CH₂ | 1 | Q8 | CF₃ |
| 13-181 | C—CH₃ | CH | C—(A)r—Q | N | CH | CH₂ | 1 | Q8 | CF₃ |
| 13-182 | C—CH₃ | CH | C—(A)r—Q | N | CH | CH₂ | 1 | Q8 | CF₃ |
| 13-183 | C—CH₃ | CH | C—(A)r—Q | N | CH | CH₂ | 1 | Q8 | CF₃ |
| 13-184 | C—CH₃ | CH | C—(A)r—Q | CH | CH | CH₂ | 1 | Q18 | H |
| 13-185 | C—CH₃ | CH | C—(A)r—Q | CH | CH | CH₂ | 1 | Q18 | H |
| 13-186 | C—CH₃ | N | C—(A)r—Q | CH | CH | CH₂ | 1 | Q8 | C₂F₅ |
| 13-187 | C—CH₃ | N | C—(A)r—Q | CH | CH | CH₂ | 1 | Q8 | C₂F₅ |
| 13-188 | C—CH₃ | N | C—(A)r—Q | CH | CH | CH₂ | 1 | Q8 | C₂F₅ |
| 13-189 | C—CH₃ | N | C—(A)r—Q | CH | CH | CH₂ | 1 | Q8 | C₂F₅ |
| 13-190 | C—CH₃ | N | C—(A)r—Q | CH | CH | CH₂ | 1 | Q8 | C₂F₅ |
| 13-191 | C—CH₃ | CH | C—(A)r—Q | CH | CH | CH₂ | 1 | Q8 | CF₃ |
| 13-192 | C—CH₃ | CH | C—(A)r—Q | CH | CH | CH₂ | 1 | Q8 | CF₃ |
| 13-193 | C—CH₃ | CH | C—(A)r—Q | CH | CH | CH₂ | 1 | Q8 | CF₃ |
| 13-194 | C—CH₃ | CH | C—(A)r—Q | CH | CH | CH₂ | 1 | Q8 | CF₃ |
| 13-195 | C—CH₃ | CH | C—(A)r—Q | CH | CH | CH₂ | 1 | Q8 | CF₃ |
| 13-196 | C—CH₃ | CH | C—(A)r—Q | CH | CH | CH₂ | 1 | Q8 | CF₃ |
| 13-197 | C—CH₃ | CH | C—(A)r—Q | CH | CH | CH₂ | 1 | Q8 | CF₃ |
| 13-198 | C—CH₃ | CH | C—(A)r—Q | CH | CH | CH₂ | 1 | Q8 | CF₃ |
| 13-199 | C—CH₂SCH₃ | CH | C—(A)r—Q | CH | CH | CH₂ | 1 | Q8 | CF₃ |
| 13-200 | C—CH₃ | CH | C—(A)r—Q | CH | CH | CH₂ | 1 | Q8 | CF₃ |
| 13-201 | C—CH₃ | CH | C—(A)r—Q | CH | CH | CH₂ | 1 | Q8 | CF₃ |
| 13-202 | C—CH₃ | CH | C—(A)r—Q | CH | CH | CH₂ | 1 | Q8 | CF₃ |
| 13-203 | C—CH₃ | CH | C—(A)r—Q | CH | CH | CH₂ | 1 | Q8 | CF₃ |
| 13-204 | C—CH₃ | CH | C—(A)r—Q | CH | CH | CH₂ | 1 | Q8 | C₂F₅ |
| 13-205 | C—CH₃ | CH | C—(A)r—Q | CH | CH | CH₂ | 1 | Q8 | C₂F₅ |
| 13-206 | C—CH₃ | CH | C—(A)r—Q | CH | CH | CH₂ | 1 | Q8 | F |
| 13-207 | C—CH₃ | CH | C—(A)r—Q | CH | CH | CH₂ | 1 | Q8 | CF₃ |
| 13-208 | C—CH₃ | CH | C—(A)r—Q | CH | CH | CH₂ | 1 | Q8 | CF₃ |
| 13-209 | C—CH₃ | CH | C—(A)r—Q | CH | CH | CH₂ | 1 | Q8 | CF₃ |
| 13-210 | C—CH₃ | CH | C—(A)r—Q | CH | CH | CH₂ | 1 | Q8 | CF₃ |
| 13-211 | C—CH₃ | CH | C—(A)r—Q | CH | CH | CH₂ | 1 | Q8 | CF₃ |
| 13-212 | C—CH₃ | CH | C—(A)r—Q | CH | CH | CH₂ | 1 | Q8 | 4-chloropheny |
| 13-213 | C—CH₃ | CH | C—(A)r—Q | CH | CH | CH₂ | 1 | Q8 | C(CH₃)₃ |
| 13-214 | C—CH₃ | CH | C—(A)r—Q | CH | CH | CH₂ | 1 | Q8 | C₂F₅ |
| 13-215 | C—CH₃ | CH | C—(A)r—Q | CH | CH | CH₂ | 1 | Q8 | C₂F₅ |
| 13-216 | C—CH₃ | CH | C—(A)r—Q | CH | CH | CH₂ | 1 | Q8 | C₂F₅ |
| 13-217 | C—CH₃ | CH | C—(A)r—Q | CH | CH | CH₂ | 1 | Q8 | C₂F₅ |
| 13-218 | C—CH₃ | CH | C—(A)r—Q | CH | CH | CH₂ | 1 | Q8 | C₂F₅ |
| 13-219 | C—CH₃ | CH | C—(A)r—Q | CH | CH | CH₂ | 1 | Q8 | C₂F₅ |
| 13-220 | C—CH₃ | CH | C—(A)r—Q | CH | CH | CH₂ | 1 | Q8 | C₂F₅ |
| 13-221 | C—CH₃ | CH | C—(A)r—Q | CH | CH | CH₂ | 1 | Q8 | C₂F₅ |
| 13-222 | C—CH₃ | CH | C—(A)r—Q | CH | CH | CH₂ | 1 | Q8 | C₂F₅ |
| 13-223 | C—CH₃ | CH | C—(A)r—Q | CH | CH | CH₂ | 1 | Q8 | 3,5- |
| | | | | | | | | | bis(trifluoromethyl)phenyl |
| 13-224 | C—CH₃ | CH | C—(A)r—Q | CH | CH | CH₂ | 1 | Q8 | 4- |
| | | | | | | | | | (difluoromethyl)phenyl |
| 13-225 | C—CH₃ | CH | C—(A)r—Q | CH | CH | CH₂ | 1 | Q8 | phenyl |
| 13-226 | C—CH₃ | N | C—(A)r—Q | CH | CH | NH | 1 | Q11 | CH₃ |
| 13-227 | C—CH₃ | N | C—(A)r—Q | CH | CH | NH | 1 | Q11 | 2,6-dichloro-4- |
| | | | | | | | | | (trifluoromethyl)phenyl |
| 13-228 | C—CH₃ | N | C—(A)r—Q | CH | CH | O | 1 | Q9 | CH₃ |
| 13-229 | C—CH₃ | CH | C—(A)r—Q | CH | CH | CO | 1 | Q11 | H |
| 13-230 | C—CH₃ | CH | C—(A)r—Q | CH | CH | CH₂ | 1 | Q11 | H |
| 13-231 | C—CH₃ | CH | C—(A)r—Q | CH | CH | CH(OH) | 1 | Q11 | H |
| 13-232 | C—Cl | N | C—(A)r—Q | CH | CH | CH₂ | 1 | Q8 | CF₃ |
| 13-233 | C—CN | CH | C—(A)r—Q | CH | CH | CH₂ | 1 | Q8 | CF₃ |
| 13-234 | C—F | CH | C—(A)r—Q | CH | CH | CH₂ | 1 | Q8 | CF₃ |
| 13-235 | C—N(CH₃)₂ | CH | C—(A)r—Q | CH | CH | CH₂ | 1 | Q8 | CF₃ |
| 13-236 | C—OCH₃ | CH | C—(A)r—Q | CH | CH | CH₂ | 1 | Q8 | CF₃ |
| 13-237 | C—SCH₃ | CH | C—(A)r—Q | CH | CH | CH₂ | 1 | Q8 | CF₃ |
| 13-238 | C—CH₃ | N | C—(A)r—Q | CH | CH | CH₂ | 1 | Q8 | CF₃ |
| 13-239 | C—CH₃ | N | C—(A)r—Q | CH | CH | CH₂ | 1 | Q8 | CF₃ |
| 13-240 | C—CH₃ | CH | C—(A)r—Q | CH | CH | CH₂ | 1 | Q8 | CF₃ |
| 13-241 | C—CH₃ | CH | C—(A)r—Q | CH | CH | CH₂ | 1 | Q8 | CF₃ |
| 13-242 | C—CH₃ | CH | C—(A)r—Q | CH | CH | CH₂ | 1 | Q8 | CF₃ |
| 13-243 | C—CN | CH | C—(A)r—Q | CH | CH | CH₂ | 1 | Q8 | CF₃ |
| 13-244 | C—CH₃ | CH | C—(A)r—Q | CH | CH | CH₂ | 1 | Q8 | C₂F₅ |
| 13-245 | C—N(CH₃)₂ | CH | C—(A)r—Q | CH | CH | CH₂ | 1 | Q8 | CF₃ |
| 13-246 | C—SCH₃ | CH | C—(A)r—Q | CH | CH | CH₂ | 1 | Q8 | CF₃ |
| 13-247 | C—CH₃ | N | C—(A)r—Q | CH | CH | CH₂ | 1 | Q8 | CF₃ |
| 13-248 | C—CH₃ | N | C—(A)r—Q | CH | CH | NH | 1 | Q11 | CH₃ |
| 13-249 | C—CH₃ | N | C—(A)r—Q | CH | CH | O | 1 | Q9 | CH₃ |
| 13-250 | C—Cl | N | C—(A)r—Q | CH | CH | CH₂ | 1 | Q8 | CF₃ |
| 13-251 | C—CH₃ | CH | C—(A)r—Q | CH | CH | CH₂ | 1 | Q8 | CF₃ |
| 13-252 | C—CH₃ | CH | C—(A)r—Q | CH | CH | CH₂ | 1 | Q8 | CF₃ |
| 13-253 | C—CH₃ | CH | C—(A)r—Q | CH | CH | CH₂ | 1 | Q8 | CF₃ |
| 13-254 | C—CH₃ | CH | C—(A)r—Q | CH | CH | CH₂ | 1 | Q8 | CF₃ |
| 13-255 | C—CH₃ | CH | C—(A)r—Q | CH | CH | CH₂ | 1 | Q8 | CF₃ |
| 13-256 | C—CH₃ | CH | C—(A)r—Q | CH | CH | CH₂ | 1 | Q8 | CF₃ |
| 13-257 | C—CH₃ | CH | C—(A)r—Q | CH | CH | CH₂ | 1 | Q8 | CF₃ |
| 13-258 | C—CH₃ | CH | C—(A)r—Q | CH | CH | CH₂ | 1 | Q8 | CF₃ |
| 13-259 | C—CH₃ | CH | C—(A)r—Q | CH | CH | CH₂ | 1 | Q8 | CF₃ |
| 13-260 | C—CH₃ | CH | C—(A)r—Q | CH | CH | CH₂ | 1 | Q8 | CF₃ |
| 13-261 | C—CH₃ | CH | C—(A)r—Q | CH | CH | CH₂ | 1 | Q8 | CF₃ |
| 13-262 | C—CH₃ | CH | C—(A)r—Q | CH | CH | CH₂ | 1 | Q8 | CF₃ |
| 13-263 | C—CH₃ | CH | C—(A)r—Q | CH | CH | CH₂ | 1 | Q8 | CF₃ |
| 13-264 | C—CH₃ | N | C—(A)r—Q | CH | CH | CH₂ | 1 | Q8 | CF₃ |
| 13-265 | C—CH₃ | CH | C—(A)r—Q | CH | CH | CH₂ | 1 | Q8 | CF₃ |
| 13-266 | C—CH₃ | N | C—(A)r—Q | CH | CH | CH₂ | 1 | Q8 | CF₃ |
| 13-267 | C—CH₃ | CH | C—(A)r—Q | CH | CH | CH₂ | 1 | Q8 | CF₃ |
| 13-268 | C—CH₃ | CH | C—(A)r—Q | CH | CH | CH₂ | 1 | Q8 | CF₃ |
| 13-269 | C—CH₃ | CH | C—(A)r—Q | CH | CH | CH₂ | 1 | Q8 | CF₃ |
| 13-270 | C—CH₃ | CH | C—(A)r—Q | CH | CH | CH₂ | 1 | Q8 | CF₃ |
| 13-271 | C—CH₃ | CH | C—(A)r—Q | CH | CH | CH₂ | 1 | Q8 | CF₃ |
| 13-272 | C—CH₃ | CH | C—(A)r—Q | CH | CH | CH₂ | 1 | Q8 | CF₃ |
| 13-273 | C—CH₃ | CH | C—(A)r—Q | CH | CH | CH₂ | 1 | Q8 | CF₃ |
| 13-274 | C—CH₃ | N | C—(A)r—Q | CH | CH | CH₂ | 1 | Q8 | CF₃ |
| 13-275 | C—CH₃ | CH | C—(A)r—Q | CH | CH | CH₂ | 1 | Q8 | CF₃ |
| 13-276 | C—CH₃ | N | C—(A)r—Q | CH | CH | CH₂ | 1 | Q8 | CF₃ |
| 13-277 | C—CH₃ | CH | C—(A)r—Q | CH | CH | CH₂ | 1 | Q8 | CF₃ |
| 13-278 | C—CH₃ | N | C—(A)r—Q | CH | CH | CH₂ | 1 | Q8 | CF₃ |
| 13-279 | C—CH₃ | N | C—(A)r—Q | CH | CH | CH₂ | 1 | Q8 | CF₃ |
| 13-280 | C—CH₃ | N | C—(A)r—Q | CH | CH | CH₂ | 1 | Q8 | CF₃ |
| 13-281 | C—CH₃ | N | C—(A)r—Q | CH | CH | CH₂ | 1 | Q8 | CF₃ |
| 13-282 | C—CH₃ | N | C—(A)r—Q | CH | CH | CH₂ | 1 | Q8 | CF₃ |
| 13-283 | C—CH₃ | N | C—(A)r—Q | CH | CH | CH₂ | 1 | Q8 | CF₃ |
| 13-284 | C—CH₃ | N | C—(A)r—Q | CH | CH | CH₂ | 1 | Q8 | CF₃ |
| 13-285 | C—CH₃ | N | C—(A)r—Q | CH | CH | CH₂ | 1 | Q8 | CF₃ |
| 13-286 | C—CH₃ | N | C—(A)r—Q | CH | CH | CH₂ | 1 | Q8 | CF₃ |
| 13-287 | C—CH₃ | N | C—(A)r—Q | CH | CH | CH₂ | 1 | Q8 | CF₃ |
| 13-288 | C—CH₃ | N | C—(A)r—Q | CH | CH | CH₂ | 1 | Q8 | CF₃ |
| 13-289 | C—CH₃ | N | C—(A)r—Q | CH | CH | CH₂ | 1 | Q8 | CF₃ |
| 13-290 | C—CH₃ | N | C—(A)r—Q | CH | CH | CH₂ | 1 | Q8 | C₂F₅ |
| 13-291 | C—CH₃ | CH | C—(A)r—Q | CH | CH | CH₂ | 1 | Q8 | CF₃ |
| 13-292 | C—CH₃ | CH | C—(A)r—Q | CH | CH | CH₂ | 1 | Q8 | C₂F₅ |
| 13-293 | C—CH₃ | CH | C—(A)r—Q | CH | CH | CH₂ | 1 | Q8 | C₂F₅ |
| 13-294 | C—CH₃ | CH | C—(A)r—Q | CH | CH | CH₂ | 1 | Q8 | C₂F₅ |
| 13-295 | C—CH₃ | CH | C—(A)r—Q | CH | CH | CH₂ | 1 | Q8 | CF₃ |
| 13-296 | C—CH₃ | CH | C—(A)r—Q | CH | CH | CH₂ | 1 | Q8 | CF₃ |
| 13-297 | C—CH₃ | CH | C—(A)r—Q | CH | CH | CH₂ | 1 | Q8 | CF₃ |
| 13-298 | C—CH₃ | CH | C—(A)r—Q | CH | CH | CH₂ | 1 | Q8 | CF₃ |
| 13-299 | C—CH₃ | CH | C—(A)r—Q | CH | CH | CH₂ | 1 | Q8 | CF₃ |
| 13-300 | C—CH₃ | CH | C—(A)r—Q | CH | CH | CH₂ | 1 | Q8 | C₂F₅ |
| 13-301 | CH | CH | C—(A)r—Q | CH | CH | CH₂ | 1 | Q8 | CF₃ |
| 13-302 | C—CH₃ | CH | C—(A)r—Q | CH | CH | CH(CH₃) | 1 | Q8 | CF₃ |
| 13-303 | C—CH₃ | CH | C—(A)r—Q | CH | CH | CH₂ | 1 | Q8 | C₂F₅ |
| 13-304 | C—CH₃ | CH | C—(A)r—Q | CH | CH | CH₂ | 1 | Q8 | C₂F₅ |
| 13-305 | C—CH₃ | CH | C—(A)r—Q | CH | CH | CH₂ | 1 | Q8 | CF₃ |
| 13-306 | C—CH₃ | CH | C—(A)r—Q | CH | CH | — | 0 | Q8 | CF₃ |
| 13-307 | C—CH₃ | CH | C—(A)r—Q | CH | CH | CH₂ | 1 | Q8 | F |
| 13-308 | C—CH₃ | CH | C—(A)r—Q | CH | CH | CH₂ | 1 | Q8 | C₂F₅ |
| 13-309 | C—CH₃ | CH | C—(A)r—Q | CH | CH | CH₂ | 1 | Q8 | C₂F₅ |
| 13-310 | C—CH₃ | CH | C—(A)r—Q | CH | CH | CH₂ | 1 | Q8 | C₂F₅ |
| 13-311 | C—CH₃ | CH | C—(A)r—Q | CH | CH | CH₂ | 1 | Q8 | C₂F₅ |
| 13-312 | C—CH₃ | CH | C—(A)r—Q | CH | CH | CH₂ | 1 | Q8 | CF₃ |
| 13-313 | C—CH₃ | CH | C—(A)r—Q | CH | CH | CH₂ | 1 | Q8 | CF₃ |
| 13-314 | C—CH₃ | CH | C—(A)r—Q | CH | CH | CH₂ | 1 | Q8 | CF₃ |
| 13-315 | C—CH₃ | CH | C—(A)r—Q | CH | CH | CH₂ | 1 | Q8 | CF₃ |
| 13-316 | C—CH₃ | CH | C—(A)r—Q | CH | CH | CH₂ | 1 | Q8 | CF₃ |
| 13-317 | C—CH₃ | CH | C—(A)r—Q | CH | CH | CH₂ | 1 | Q8 | CF₃ |
| 13-318 | C—CH₃ | CH | C—(A)r—Q | CH | CH | CH₂ | 1 | Q8 | CF₃ |
| 13-319 | C—CH₃ | CH | C—(A)r—Q | CH | CH | CH₂ | 1 | Q8 | CF₃ |
| 13-320 | C—CH₃ | CH | C—(A)r—Q | CH | CH | CH₂ | 1 | Q8 | CF₃ |
| 13-321 | C—CH₃ | CH | C—(A)r—Q | CH | CH | CH₂ | 1 | Q8 | CF₃ |
| 13-322 | C—CH₃ | CH | C—(A)r—Q | CH | CH | CH₂ | 1 | Q8 | CF₃ |
| 13-323 | C—CH₃ | CH | C—(A)r—Q | CH | CH | CH₂ | 1 | Q8 | CF₃ |
| 13-324 | C—CH₃ | CH | C—(A)r—Q | CH | CH | CH₂ | 1 | Q8 | CF₃ |
| 13-325 | C—CH₃ | CH | C—(A)r—Q | CH | CH | CH₂ | 1 | Q8 | C₂F₅ |
| 13-326 | C—CH₃ | CH | C—(A)r—Q | CH | CH | CH₂ | 1 | Q8 | 3,5- |
| | | | | | | | | | bis(trifluoromethyl)phenyl |
| 13-327 | C—CH₃ | CH | C—(A)r—Q | CH | CH | CH₂ | 1 | Q8 | 3,5- |
| | | | | | | | | | bis(trifluoromethyl)phenyl |
| 13-328 | C—CH₃ | CH | C—(A)r—Q | CH | CH | CH₂ | 1 | Q8 | 4-chloropheny |
| 13-329 | C—CH₃ | CH | C—(A)r—Q | CH | CH | CH₂ | 1 | Q8 | C(CH₃)₃ |
| 13-330 | C—CH₃ | CH | C—(A)r—Q | CH | CH | CH₂ | 1 | Q8 | 4- |
| | | | | | | | | | (difluoromethoxy)phenyl |
| 13-331 | C—CH₃ | CH | C—(A)r—Q | CH | CH | CH₂ | 1 | Q8 | 4- |
| | | | | | | | | | (difluoromethoxy)phenyl |
| 13-332 | C—CH₃ | CH | C—(A)r—Q | CH | CH | CH₂ | 1 | Q8 | C₂F₅ |
| 13-333 | C—CH₃ | CH | C—(A)r—Q | CH | CH | CH₂ | 1 | Q8 | C₂F₅ |
| 13-334 | C—CH₃ | CH | C—(A)r—Q | CH | CH | CH₂ | 1 | Q8 | C₂F₅ |
| 13-335 | C—CH₃ | CH | C—(A)r—Q | CH | CH | CH₂ | 1 | Q8 | C₂F₅ |
| 13-336 | C—CH₃ | CH | C—(A)r—Q | CH | CH | CH₂ | 1 | Q8 | C₂F₅ |
| 13-337 | C—CH₃ | CH | C—(A)r—Q | CH | CH | CH₂ | 1 | Q8 | C₂F₅ |
| 13-338 | C—CH₃ | CH | C—(A)r—Q | CH | CH | CH₂ | 1 | Q8 | C₂F₅ |
| 13-339 | C—CH₃ | CH | C—(A)r—Q | CH | CH | CH₂ | 1 | Q8 | C₂F₅ |
| 13-340 | C—CH₃ | CH | C—(A)r—Q | CH | CH | CH(OH) | 1 | Q11 | H |
| 13-341 | C—CH₃ | CH | C—(A)r—Q | CH | CH | CH(OH) | 1 | Q11 | H |
| 13-342 | C—CH₃ | N | C—(A)r—Q | CH | CH | CH₂ | 1 | Q8 | C₂F₅ |
| 13-343 | C—CH₃ | N | C—(A)r—Q | CH | CH | CH₂ | 1 | Q8 | C₂F₅ |
| 13-344 | C—CH₃ | CH | C—(A)r—Q | CH | CH | CH₂ | 1 | Q8 | CF₃ |
| 13-345 | C—CH₃ | CH | C—(A)r—Q | N | CH | CH₂ | 1 | Q8 | CF₃ |
| 13-346 | C—CH₃ | CH | C—(A)r—Q | CH | CH | CH₂ | 1 | Q8 | CF₃ |
| 13-347 | C—OCH₃ | CH | C—(A)r—Q | CH | CH | CH₂ | 1 | Q8 | CF₃ |
| 13-348 | C—CH₂SCH₃ | CH | C—(A)r—Q | CH | CH | CH₂ | 1 | Q8 | CF₃ |
| 13-349 | C—CH₃ | CH | C—(A)r—Q | CH | CH | CH₂ | 1 | Q8 | CF₃ |
| 13-350 | CH | C—CF₃ | C—(A)r—Q | CH | CH | — | 0 | Q8 | CF₃ |
| 13-351 | C—CH₃ | CH | C—(A)r—Q | CH | CH | CO | 1 | Q12 | H |
| 13-352 | C—CH₃ | CH | C—(A)r—Q | CH | CH | CO | 1 | Q12 | H |
| 13-353 | C—CH₃ | N | C—(A)r—Q | CH | CH | N—CH₃ | 1 | Q11 | CH₃ |
| 13-354 | C—CH₃ | N | C—(A)r—Q | CH | CH | N—CH₃ | 1 | Q11 | CH₃ |
| 13-355 | C—CH₃ | CH | C—(A)r—Q | CH | CH | CH₂ | 1 | Q8 | CF₃ |
| 13-356 | C—CH₃ | CH | C—(A)r—Q | CH | CH | CH₂ | 1 | Q8 | CF₃ |
| 13-357 | C—CH₃ | CH | C—(A)r—Q | CH | CH | CH₂ | 1 | Q8 | CF₃ |
| 13-358 | C—CH₃ | CH | C—(A)r—Q | CH | CH | CH₂ | 1 | Q8 | CF₃ |
| 13-359 | C—CH₃ | CH | C—(A)r—Q | CH | CH | CH₂ | 1 | Q8 | CF₃ |
| 13-360 | C—CH₃ | CH | C—(A)r—Q | CH | CH | CH₂ | 1 | Q8 | CF₃ |
| 13-361 | C—CH₃ | CH | C—(A)r—Q | CH | CH | CH₂ | 1 | Q8 | CF₃ |
| 13-362 | C—CH₃ | CH | C—(A)r—Q | CH | CH | CH₂ | 1 | Q20 | H |
| 13-363 | C—CH₃ | N | C—(A)r—Q | CH | CH | CH₂ | 1 | Q18 | CF₃ |
| 13-364 | C—CH₃ | N | C—(A)r—Q | CH | CH | CH₂ | 1 | Q18 | CF₃ |
| 13-365 | C—CH₃ | CH | C—(A)r—Q | CH | CH | COCH₂ | 1 | Q8 | CF₃ |
| 13-366 | C—CH₃ | CH | C—(A)r—Q | CH | CH | CH(OH)CH₂ | 1 | Q8 | CF₃ |
| 13-367 | C—CH₃ | CH | C—(A)r—Q | CH | CH | CH(CH3) | 1 | Q8 | CF₃ |
| 13-368 | C—CH₃ | CH | C—(A)r—Q | CH | CH | CH₂ | 1 | Q8 | H |
| 13-369 | C—CH₃ | CH | C—(A)r—Q | CH | CH | CH₂ | 1 | Q8 | H |
| 13-370 | C—CH₃ | CH | C—(A)r—Q | CH | CH | CH₂ | 1 | Q8 | H |
| 13-371 | C—CH₃ | CH | C—(A)r—Q | CH | CH | CH₂ | 1 | Q8 | H |
| 13-372 | C—CH₃ | CH | C—(A)r—Q | CH | CH | CH₂ | 1 | Q8 | H |
| 13-373 | C—CH₃ | CH | C—(A)r—Q | CH | CH | CH₂ | 1 | Q8 | H |
| 13-374 | C—CH₃ | CH | C—(A)r—Q | CH | CH | CH₂ | 1 | Q8 | CF₃ |
| 13-375 | C—CH₃ | CH | C—(A)r—Q | CH | CH | CH₂ | 1 | Q8 | CF₃ |
| 13-376 | C—CH₃ | CH | C—(A)r—Q | CH | CH | CH₂ | 1 | Q8 | CF₃ |
| 13-377 | C—CH₃ | CH | C—(A)r—Q | CH | CH | CH₂ | 1 | Q8 | CF₃ |
| 13-378 | C—CH₃ | CH | C—(A)r—Q | CH | CH | CH₂ | 1 | Q8 | CF₃ |
| 13-379 | C—CH₃ | CH | C—(A)r—Q | CH | CH | CH₂ | 1 | Q8 | CF₃ |
| 13-380 | C—CH₃ | CH | C—(A)r—Q | CH | CH | CH₂ | 1 | Q8 | CF₃ |
| 13-381 | C—CH₃ | CH | C—(A)r—Q | CH | CH | CH₂ | 1 | Q8 | CF₃ |
| 13-382 | C—CH₃ | CH | C—(A)r—Q | CH | CH | CH₂ | 1 | Q8 | CF₃ |
| 13-383 | C—CH₃ | CH | C—(A)r—Q | CH | CH | CH₂ | 1 | Q8 | CF₃ |
| 13-384 | C—CH₃ | CH | C—(A)r—Q | CH | CH | CH₂ | 1 | Q8 | CF₃ |
| 13-385 | C—CH₃ | CH | C—(A)r—Q | CH | CH | CH₂ | 1 | Q8 | CF₃ |
| 13-386 | C—CH₃ | CH | C—(A)r—Q | CH | CH | CH₂ | 1 | Q8 | CF₃ |
| 13-387 | C—CH₃ | CH | C—(A)r—Q | CH | CH | CH₂ | 1 | Q8 | CF₃ |
| 13-388 | C—CH₃ | N | C—(A)r—Q | CH | CH | CH₂ | 1 | Q8 | CF₃ |
| 13-389 | C—CH₃ | CH | C—(A)r—Q | CH | CH | CH₂ | 1 | Q8 | CF₃ |
| 13-390 | C—CH₃ | CH | C—(A)r—Q | CH | CH | CH₂ | 1 | Q8 | CF₃ |
| 13-391 | C—CH₃ | CH | C—(A)r—Q | CH | CH | CH₂ | 1 | Q8 | CF₃ |
| 13-392 | C—CH₃ | CH | C—(A)r—Q | CH | CH | CH₂ | 1 | Q8 | CF₃ |
| 13-393 | C—CH₃ | CH | C—(A)r—Q | CH | CH | CH₂ | 1 | Q8 | CF₃ |
| 13-394 | C—CH₃ | CH | C—(A)r—Q | CH | CH | CH₂ | 1 | Q8 | CF₃ |
| 13-395 | C—CH₃ | CH | C—(A)r—Q | CH | CH | CH₂ | 1 | Q8 | CF₃ |
| 13-396 | C—CH₃ | CH | C—(A)r—Q | CH | CH | CH₂ | 1 | Q8 | CF₃ |
| 13-397 | C—CH₃ | CH | C—(A)r—Q | CH | CH | CH₂ | 1 | Q8 | CF₃ |
| 13-398 | C—CH₃ | CH | C—(A)r—Q | CH | CH | CH₂ | 1 | Q8 | CF₃ |
| 13-399 | C—CH₃ | CH | C—(A)r—Q | CH | CH | CH₂ | 1 | Q8 | CF₃ |
| 13-400 | C—CH₃ | CH | C—(A)r—Q | CH | CH | CH₂ | 1 | Q8 | CF₃ |
| 13-401 | C—CH₃ | CH | C—(A)r—Q | CH | CH | CH₂ | 1 | Q8 | CF₃ |
| 13-402 | C—CH₃ | CH | C—(A)r—Q | CH | CH | CH₂ | 1 | Q8 | CF₃ |
| 13-403 | C—CH₃ | CH | C—(A)r—Q | CH | CH | CH₂ | 1 | Q8 | CF₃ |
| 13-404 | C—CH₃ | CH | C—(A)r—Q | CH | CH | CH₂ | 1 | Q8 | CF₃ |
| 13-405 | C—CH₃ | CH | C—(A)r—Q | CH | CH | CH₂ | 1 | Q8 | CF₃ |
| 13-406 | C—CH₃ | CH | C—(A)r—Q | CH | CH | CH₂ | 1 | Q8 | CF₃ |
| 13-407 | C—CH₃ | CH | C—(A)r—Q | CH | CH | CH₂ | 1 | Q8 | CF₃ |
| 13-408 | C—CH₃ | CH | C—(A)r—Q | CH | CH | CH₂ | 1 | Q8 | CF₃ |
| 13-409 | C—CH₃ | CH | C—(A)r—Q | CH | CH | CH₂ | 1 | Q8 | CF₃ |
| 13-410 | C—CH₃ | CH | C—(A)r—Q | CH | CH | CH₂ | 1 | Q8 | CF₃ |
| 13-436 | C—SO₂CH₃ | CH | C—(A)r—Q | CH | CH | CH₂ | 1 | Q8 | CF₃ |
| 13-437 | C—SO₂CH₃ | CH | C—(A)r—Q | CH | CH | CH₂ | 1 | Q8 | CF₃ |
| 13-438 | C—OCH₂CF₃ | CH | C—(A)r—Q | CH | CH | CH₂ | 1 | Q8 | CF₃ |
| 13-439 | C—OCH₂CF₃ | CH | C—(A)r—Q | CH | CH | CH₂ | 1 | Q8 | CF₃ |
| 13-440 | C—SCH₂CF₃ | CH | C—(A)r—Q | CH | CH | CH₂ | 1 | Q8 | CF₃ |
| 13-441 | C—SCH₂CF₃ | CH | C—(A)r—Q | CH | CH | CH₂ | 1 | Q8 | CF₃ |
| 13-442 | C—NHCH(CH₃)₂ | CH | C—(A)r—Q | CH | CH | CH₂ | 1 | Q8 | CF₃ |
| 13-443 | C—NHCH(CH₃)₂ | CH | C—(A)r—Q | CH | CH | CH₂ | 1 | Q8 | CF₃ |
| 13-444 | C—CH═CH₂ | CH | C—(A)r—Q | CH | CH | CH₂ | 1 | Q8 | CF₃ |
| 13-445 | C—CH═CH₂ | CH | C—(A)r—Q | CH | CH | CH₂ | 1 | Q8 | CF₃ |
| 13-446 | C—OCH₂CH═CH₂ | CH | C—(A)r—Q | CH | CH | CH₂ | 1 | Q8 | CF₃ |
| 13-447 | C—OCH₂CH═CH₂ | CH | C—(A)r—Q | CH | CH | CH₂ | 1 | Q8 | CF₃ |
| 13-448 | C—C₂H₅ | CH | C—(A)r—Q | CH | CH | CH₂ | 1 | Q8 | CF₃ |
| 13-449 | C—C₂H₅ | CH | C—(A)r—Q | CH | CH | CH₂ | 1 | Q8 | CF₃ |
| 13-450 | C—C≡CH | CH | C—(A)r—Q | CH | CH | CH₂ | 1 | Q8 | CF₃ |
| 13-451 | C—C≡CH | CH | C—(A)r—Q | CH | CH | CH₂ | 1 | Q8 | CF₃ |
| 13-452 | C—OCH₂C≡CH | CH | C—(A)r—Q | CH | CH | CH₂ | 1 | Q8 | CF₃ |
| 13-453 | C—OCH₂C≡CH | CH | C—(A)r—Q | CH | CH | CH₂ | 1 | Q8 | CF₃ |
| 13-454 | C-cyclopropyl | CH | C—(A)r—Q | CH | CH | CH₂ | 1 | Q8 | CF₃ |
| 13-455 | C-cyclopropyl | CH | C—(A)r—Q | CH | CH | CH₂ | 1 | Q8 | CF₃ |
| 13-456 | C—O-cyclopentyl | CH | C—(A)r—Q | CH | CH | CH₂ | 1 | Q8 | CF₃ |
| 13-457 | C—O-cyclopentyl | CH | C—(A)r—Q | CH | CH | CH₂ | 1 | Q8 | CF₃ |
| 13-458 | C—CH₂-cyclopentyl | CH | C—(A)r—Q | CH | CH | CH₂ | 1 | Q8 | CF₃ |
| 13-459 | C—CH₂-cyclopentyl | CH | C—(A)r—Q | CH | CH | CH₂ | 1 | Q8 | CF₃ |
| 13-460 | C—OCH₂-cyclopropyl | CH | C—(A)r—Q | CH | CH | CH₂ | 1 | Q8 | CF₃ |
| 13-461 | C—OCH₂-cyclopropyl | CH | C—(A)r—Q | CH | CH | CH₂ | 1 | Q8 | CF₃ |
| 13-462 | C-phenyl | CH | C—(A)r—Q | CH | CH | CH₂ | 1 | Q8 | CF₃ |
| 13-463 | C-phenyl | CH | C—(A)r—Q | CH | CH | CH₂ | 1 | Q8 | CF₃ |
| 13-464 | C—O-phenyl | CH | C—(A)r—Q | CH | CH | CH₂ | 1 | Q8 | CF₃ |
| 13-465 | C—O-phenyl | CH | C—(A)r—Q | CH | CH | CH₂ | 1 | Q8 | CF₃ |
| 13-466 | C—S-phenyl | CH | C—(A)r—Q | CH | CH | CH₂ | 1 | Q8 | CF₃ |
| 13-467 | C—S-phenyl | CH | C—(A)r—Q | CH | CH | CH₂ | 1 | Q8 | CF₃ |
| 13-468 | C—CH₂-phenyl | CH | C—(A)r—Q | CH | CH | CH₂ | 1 | Q8 | CF₃ |
| 13-469 | C—CH₂-phenyl | CH | C—(A)r—Q | CH | CH | CH₂ | 1 | Q8 | CF₃ |
| 13-470 | C—OCH₂-phenyl | CH | C—(A)r—Q | CH | CH | CH₂ | 1 | Q8 | CF₃ |
| 13-471 | C—OCH₂-phenyl | CH | C—(A)r—Q | CH | CH | CH₂ | 1 | Q8 | CF₃ |
| 13-472 | C—SCH₂-phenyl | CH | C—(A)r—Q | CH | CH | CH₂ | 1 | Q8 | CF₃ |
| 13-473 | C—SCH₂-phenyl | CH | C—(A)r—Q | CH | CH | CH₂ | 1 | Q8 | CF₃ |
| 13-474 | C-pyridin-4-yl | CH | C—(A)r—Q | CH | CH | CH₂ | 1 | Q8 | CF₃ |
| 13-475 | C-pyridin-4-yl | CH | C—(A)r—Q | CH | CH | CH₂ | 1 | Q8 | CF₃ |
| 13-476 | C-[3-(CF₃)-pyrazol-1-yl] | CH | C—(A)r—Q | CH | CH | CH₂ | 1 | Q8 | CF₃ |
| 13-477 | C-[3-(CF₃)-pyrazol-1-yl] | CH | C—(A)r—Q | CH | CH | CH₂ | 1 | Q8 | CF₃ |
| 13-478 | C—O-pyridin-2-yl | CH | C—(A)r—Q | CH | CH | CH₂ | 1 | Q8 | CF₃ |
| 13-479 | C—O-pyridin-2-yl | CH | C—(A)r—Q | CH | CH | CH₂ | 1 | Q8 | CF₃ |
| 13-480 | C—S-pyridin-2-yl | CH | C—(A)r—Q | CH | CH | CH₂ | 1 | Q8 | CF₃ |
| 13-481 | C—S-pyridin-2-yl | CH | C—(A)r—Q | CH | CH | CH₂ | 1 | Q8 | CF₃ |
| 13-482 | C—OCH₂-pyridin-2-yl | CH | C—(A)r—Q | CH | CH | CH₂ | 1 | Q8 | CF₃ |
| 13-483 | C—OCH₂-pyridin-2-yl | CH | C—(A)r—Q | CH | CH | CH₂ | 1 | Q8 | CF₃ |
| 13-484 | C—O-(2-chlorophenyl) | CH | C—(A)r—Q | CH | CH | CH₂ | 1 | Q8 | CF₃ |
| 13-485 | C—O-(2-methylphenyl) | CH | C—(A)r—Q | CH | CH | CH₂ | 1 | Q8 | CF₃ |
| 13-486 | C—O-(2- | CH | C—(A)r—Q | CH | CH | CH₂ | 1 | Q8 | CF₃ |
| | cyanophenyl) | | | | | | | | |
| 13-487 | C—O-[3- | CH | C—(A)r—Q | CH | CH | CH₂ | 1 | Q8 | CF₃ |
| | (trifluoromethyl)phenyl) | | | | | | | | |
| 13-488 | C—O-[3- | CH | C—(A)r—Q | CH | CH | CH₂ | 1 | Q8 | CF₃ |
| | (trifluoromethyl)phenyl) | | | | | | | | |
| 13-489 | C—O-[4- | CH | C—(A)r—Q | CH | CH | CH₂ | 1 | Q8 | CF₃ |
| | (methoxycarbonyl)phenyl] | | | | | | | | |
| 13-490 | C—O-(4- | CH | C—(A)r—Q | CH | CH | CH₂ | 1 | Q8 | CF₃ |
| | acetylphenyl) | | | | | | | | |
| 13-491 | C—O-(4- | CH | C—(A)r—Q | CH | CH | CH₂ | 1 | Q8 | CF₃ |
| | formylphenyl) | | | | | | | | |
| 13-492 | C—O-(4- | CH | C—(A)r—Q | CH | CH | CH₂ | 1 | Q8 | CF₃ |
| | methoxyphenyl) | | | | | | | | |
| 13-493 | C—O-(4- | CH | C—(A)r—Q | CH | CH | CH₂ | 1 | Q8 | CF₃ |
| | methylthiophenyl) | | | | | | | | |
| 13-494 | C—O-[4- | CH | C—(A)r—Q | CH | CH | CH₂ | 1 | Q8 | CF₃ |
| | (trifluoromethylthio)phenyl] | | | | | | | | |
| 13-495 | C—CH₃ | CH | C—(A)r—Q | CH | CH | CH₂ | 1 | Q8 | C₂F₅ |
| 13-496 | C—CH₃ | CH | C—(A)r—Q | CH | CH | CH₂ | 1 | Q8 | C₂F₅ |
| 13-497 | C—CH₃ | CH | C—(A)r—Q | CH | CH | CH₂ | 1 | Q8 | C₂F₅ |
| 13-498 | C—CH₃ | CH | C—(A)r—Q | CH | CH | CH₂ | 1 | Q8 | C₂F₅ |
| 13-499 | C—CH₃ | CH | C—(A)r—Q | CH | CH | CH₂ | 1 | Q8 | C₂F₅ |
| 13-500 | C—CH₃ | CH | C—(A)r—Q | CH | CH | CH₂ | 1 | Q8 | C₂F₅ |
| 13-501 | C—CH₃ | CH | C—(A)r—Q | CH | CH | CH₂ | 1 | Q8 | CONHCH₂CF₃ |
| 13-502 | C—CH₃ | N | C—(A)r—Q | CH | CH | CH₂ | 1 | Q18 | CF₃ |
| 13-503 | C—CH₃ | CH | C—(A)r—Q | CH | CH | CH₂ | 1 | Q8 | CF₃ |
| 13-504 | C—CH₃ | CH | C—(A)r—Q | CH | CH | CH₂ | 1 | Q8 | CF₃ |
| 13-505 | C—CH₃ | CH | C—(A)r—Q | CH | CH | CH₂ | 1 | Q8 | CF₃ |
| 13-506 | C—CH₃ | CH | C—(A)r—Q | CH | CH | CH₂ | 1 | Q8 | CF₃ |
| 13-507 | C—CH3 | CH | C—(A)r—Q | CH | CH | CH2 | 1 | Q8 | CF3 |

| No. | R⁵ | R⁶ | R⁷ | Physical property |
|---|---|---|---|---|
| 13-1 | H | CF₃ | — | mp 170-172° C. |
| 13-2 | H | CF₃ | — | mp 98-100° C. |
| 13-3 | C₂F₅ | H | — | NMR |
| 13-4 | H | CF₃ | — | |
| 13-5 | C₂F₅ | H | — | NMR |
| 13-6 | H | CF₃ | — | |
| 13-7 | H | CF₃ | — | mp 164-165° C. |
| 13-8 | H | CF₃ | — | NMR |
| 13-9 | H | C₂F₅ | — | |
| 13-10 | H | CF₃ | — | mp 172-173° C. |
| 13-11 | H | CF₃ | — | NMR |
| 13-12 | H | CF₃ | — | |
| 13-13 | H | CF₃ | — | mp 158-160° C. |
| 13-14 | H | CF₃ | — | mp 196-198° C. |
| 13-15 | H | CF₃ | — | |
| 13-16 | H | CF₃ | — | |
| 13-17 | C₂F₅ | H | — | NMR |
| 13-18 | H | CF₃ | — | NMR |
| 13-19 | C₂F₅ | H | — | NMR |
| 13-20 | H | CF₃ | — | NMR |
| 13-21 | H | CF₃ | — | NMR |
| 13-22 | H | CF₃ | — | NMR |
| 13-23 | H | CF₃ | — | NMR |
| 13-24 | I | C₂F₅ | — | NMR |
| 13-25 | 3,5- | H | — | NMR |
| | bis(trifluoromethyl)phenyl | | | |
| 13-26 | 3,5- | H | — | NMR |
| | bis(trifluoromethyl)phenyl | | | |
| 13-27 | Cl | CHF₂ | — | logP (acid) |
| | | | | 3.21 |
| 13-28 | Br | CHF₂ | — | logP (acid) |
| | | | | 3.20 |
| 13-29 | H | H | — | NMR |
| 13-30 | H | CF₃ | — | NMR |
| 13-31 | CF₂CO₂C₂H₅ | H | — | NMR |
| 13-32 | CF₃ | C₂F₅ | — | |
| 13-33 | H | CF₃ | — | NMR |
| 13-34 | H | CF₃ | — | NMR |
| 13-35 | 4- | H | — | NMR |
| | (trifluoromethyl)phenyl | | | |
| 13-36 | 3,5- | CF₃ | — | NMR |
| | bis(trifluoromethyl)phenyl | | | |
| 13-37 | H | 3- | — | NMR |
| | | (trifluoromethyl)phenyl | | |
| 13-38 | H | H | — | NMR |
| 13-39 | Cl | CH₃ | — | logP (acid) |
| | | | | 4.24 |
| 13-40 | H | H | — | NMR |
| 13-41 | I | I | — | NMR |
| 13-42 | C≡CSi(CH₃)₃ | H | — | mp 69-72° C. |
| 13-43 | I | H | — | mp 90-94° C. |
| 13-44 | C₂H₅ | H | — | NMR |
| 13-45 | C₂F₅ | H | — | NMR |
| 13-46 | C₂F₅ | H | — | NMR |
| 13-47 | H | OCHF₂ | — | NMR |
| 13-48 | H | SCF₃ | — | NMR |
| 13-49 | 3,5- | H | — | NMR |
| | bis(trifluoromethyl)phenyl | | | |
| 13-50 | C₂F₅ | H | — | logP (acid) |
| | | | | 4.27 |
| 13-51 | H | H | — | NMR |
| 13-52 | I | H | — | NMR |
| 13-53 | C₂F₅ | H | — | NMR |
| 13-54 | H | CF₃ | — | NMR |
| 13-55 | H | H | — | NMR |
| 13-56 | I | H | — | NMR |
| 13-57 | C₂F₅ | H | — | NMR |
| 13-58 | Cl | CHF₂ | — | logP (acid) |
| | | | | 2.13 |
| 13-59 | H | CF₃ | — | NMR |
| 13-60 | 4- | H | — | NMR |
| | (trifluoromethyl)phenyl | | | |
| 13-61 | H | 3- | — | mp 88-93° C. |
| | | (trifluoromethyl)phenyl | | |
| 13-62 | H | H | — | |
| 13-63 | H | H | — | |
| 13-64 | CF₃ | H | — | NMR |
| 13-65 | I | I | — | mp 226-227° C. |
| 13-66 | I | H | — | NMR |
| 13-67 | H | OCHF₂ | — | NMR |
| 13-68 | H | SCF₃ | — | NMR |
| 13-69 | H | C₂F₅ | — | logP (acid) |
| | | | | 3.02 |
| 13-70 | H | CF₃ | — | logP (acid) |
| | | | | 2.86 |
| 13-71 | H | H | — | NMR |
| 13-72 | I | H | — | mp 187-191° C. |
| 13-73 | H | CF₃ | — | mp 220-222° C. |
| 13-74 | H | H | — | mp 185-190° C. |
| 13-75 | I | H | — | mp 215-219° C. |
| 13-76 | Cl | CHF₂ | — | logP (acid) |
| | | | | 2.48 |
| 13-77 | CN | H | — | NMR |
| 13-78 | CN | H | — | NMR |
| 13-79 | CN | H | — | |
| 13-80 | CN | H | — | NMR |
| 13-81 | CN | H | — | |
| 13-82 | CN | H | — | NMR |
| 13-83 | CN | H | — | |
| 13-84 | CN | H | — | NMR |
| 13-85 | 4- | H | — | NMR |
| | (trifluoromethyl)phenyl | | | |
| 13-86 | H | 3- | — | mp 102-105° C. |
| | | (trifluoromethyl)phenyl | | |
| 13-87 | H | H | — | |
| 13-88 | H | H | — | |
| 13-89 | CF₃ | H | — | NMR |
| 13-90 | I | I | — | NMR |
| 13-91 | Cl | H | — | |
| 13-92 | Cl | H | — | |
| 13-93 | Cl | H | — | |
| 13-94 | Br | H | — | NMR |
| 13-95 | Br | H | — | |
| 13-96 | Br | H | — | |
| 13-97 | Br | H | — | |
| 13-98 | Br | H | — | |
| 13-99 | Br | H | — | |
| 13-100 | Br | H | — | |
| 13-101 | Br | H | — | |
| 13-102 | Br | H | — | |
| 13-103 | Br | H | — | |
| 13-104 | Br | H | — | |
| 13-105 | Br | H | — | |
| 13-106 | Br | H | — | |
| 13-107 | I | H | — | NMR |
| 13-108 | H | OCHF₂ | — | NMR |
| 13-109 | H | SCF₃ | — | NMR |
| 13-110 | C₂F₅ | H | — | logP (acid) 3.46 |
| 13-111 | H | H | — | NMR |
| 13-112 | H | CF₃ | — | NMR |
| 13-113 | H | H | — | mp 172-177° C. |
| 13-114 | H | OCHF₂ | — | |
| 13-115 | H | CF₃ | — | NMR |
| 13-116 | H | CF₃ | — | mp 179-184° C. |
| 13-117 | H | CF₃ | — | |
| 13-118 | H | CF₃ | — | |
| 13-119 | H | CF₃ | — | |
| 13-120 | H | CF₃ | — | NMR |
| 13-121 | H | CF₃ | — | NMR |
| 13-122 | H | CF₃ | — | NMR |
| 13-123 | H | CF₃ | — | mp 206-207° C. |
| 13-124 | H | — | — | NMR |
| 13-125 | H | — | — | NMR |
| 13-126 | Cl | CHF₂ | — | logP (acid) 3.28 |
| 13-127 | H | H | — | NMR |
| 13-128 | H | CF₃ | — | logP (neutral) |
| | | | | 4.42 |
| 13-129 | I | H | — | |
| 13-130 | H | CF₃ | — | |
| 13-131 | H | C₂F₅ | — | |
| 13-132 | C₂F₅ | H | — | mp 82-88° C. |
| 13-133 | H | OCHF₂ | — | |
| 13-134 | C₂F₅ | H | — | logP (acid) 4.35 |
| 13-135 | H | CF₃ | — | logP (acid) 4.30 |
| 13-136 | H | CF₃ | — | logP (acid) 3.08 |
| 13-137 | C₂F₅ | H | — | mp 127-129° C. |
| 13-138 | C₂F₅ | H | — | logP (acid) 3.03 |
| 13-139 | H | CF₃ | — | logP (acid) 3.56 |
| 13-140 | C₂F₅ | H | — | mp 134-136° C. |
| 13-141 | C₂F₅ | H | — | logP (acid) 3.52 |
| 13-142 | H | CF₃ | — | |
| 13-143 | H | CF₃ | — | mp 172-174° C. |
| 13-144 | H | CF₃ | — | mp 189-192° C. |
| 13-145 | Cl | CHF₂ | — | logP (acid) 3.34 |
| 13-146 | CH(OH)CF₃ | H | — | NMR |
| 13-147 | 2,2,2-trifluoro-N- | H | — | NMR |
| | methoxyethanimidoyl | | | |
| 13-148 | H | CF₃ | — | NMR |
| 13-149 | H | CF₃ | — | NMR |
| 13-150 | H | CF₃ | — | NMR |
| 13-151 | H | CF₃ | — | mp 155-159° C. |
| 13-152 | H | CF₃ | — | mp 177-179° C. |
| 13-153 | I | H | — | |
| 13-154 | C₂F₅ | H | — | |
| 13-155 | H | OCHF₂ | — | |
| 13-156 | C₂F₅ | H | — | logP (acid) 4.46 |
| 13-157 | H | CF₃ | — | logP (acid) 4.39 |
| 13-158 | H | CF₃ | — | NMR |
| 13-159 | C₂F₅ | H | — | logP (neutral) |
| | | | | 3.08 |
| 13-160 | H | CF₃ | — | mp 204-206° C. |
| 13-161 | H | CF₃ | — | mp 162-164° C. |
| 13-162 | H | H | — | mp 89-91° C. |
| 13-163 | H | CF₃ | — | |
| 13-164 | H | CF₃ | — | |
| 13-165 | H | CF₃ | — | |
| 13-166 | C₂F₅ | H | CH(OH)CF₃ | NMR |
| 13-167 | H | CF₃ | — | mp 162-163° C. |
| 13-168 | H | CF₃ | — | |
| 13-169 | H | CF₃ | — | |
| 13-170 | H | CF₃ | — | |
| 13-171 | H | CF₃ | — | |
| 13-172 | H | CF₃ | — | NMR |
| 13-173 | H | CF₃ | — | NMR |
| 13-174 | H | CF₃ | — | |
| 13-175 | H | CF₃ | — | logP (acid) 3.52 |
| 13-176 | H | CF₃ | — | |
| 13-177 | H | CF₃ | — | |
| 13-178 | H | CF₃ | — | logP (acid) 3.59 |
| 13-179 | H | CF₃ | — | |
| 13-180 | H | CF₃ | — | |
| 13-181 | H | CF₃ | — | logP (acid) 3.67 |
| 13-182 | H | CF₃ | — | logP (acid) 2.3 |
| 13-183 | H | CF₃ | — | |
| 13-184 | CF₃ | H | — | |
| 13-185 | CF₃ | H | — | |
| 13-186 | H | CF₃ | — | logP (acid) 3.12 |
| 13-187 | H | CF₃ | — | logP (acid) 3.19 |
| 13-188 | H | CF₃ | — | logP (acid) 2.81 |
| 13-189 | H | CF₃ | — | logP (acid) 3.27 |
| 13-190 | H | CF₃ | — | logP (acid) 2.76 |
| 13-191 | H | CF₃ | — | logP (acid) 1.81 |
| 13-192 | H | CF₃ | — | NMR |
| 13-193 | H | CF₃ | — | NMR |
| 13-194 | H | CF₃ | — | NMR |
| 13-195 | H | CF₃ | — | NMR |
| 13-196 | H | CF₃ | — | NMR |
| 13-197 | H | CF₃ | — | NMR |
| 13-198 | H | CF₃ | — | logP (acid) 3.32 |
| 13-199 | H | CF₃ | — | NMR |
| 13-200 | H | NH₂ | — | logP (acid) 2.31 |
| 13-201 | 5-(3,5- | H | — | logP (acid) 3.43 |
| | dichlorophenyl)-5- | | | |
| | (trifluoromethyl)-4,5- | | | |
| | dihydroisoxazol-3-yl | | | |
| 13-202 | CO₂C₂H₅ | H | — | logP (acid) 3.43 |
| 13-203 | H | SCF₃ | — | NMR |
| 13-204 | H | SCH₂CF₃ | — | NMR |
| 13-205 | COC₂F₅ | H | — | logP (acid) 4.46 |
| 13-206 | CF₃ | C₂F₅ | — | NMR |
| 13-207 | dimethylcarbamoyl | H | — | logP (acid) 2.56 |
| 13-208 | (1,1,1- | H | — | logP (acid) 3.25 |
| | trifluoropropan-2- | | | |
| | yl)carbamoyl | | | |
| 13-209 | propan- | H | — | logP (acid) 2.87 |
| | 2-ylcarbamoyl | | | |
| 13-210 | (2,2,2- | H | — | logP (acid) 3.05 |
| | trifluoroethyl)carbamoyl | | | |
| 13-211 | [3,5- | H | — | logP (acid) 4.55 |
| | bis(trifluoromethyl)phenyl]carbamoyl | | | |
| 13-212 | 3-(trifluoromethyl)- | H | — | NMR |
| | 1,2,4-triazol-1-yl | | | |
| 13-213 | 3-(trifluoromethyl)- | H | — | NMR |
| | 1,2,4-triazol-1-yl | | | |
| 13-214 | [3,5- | | | |
| | bis(trifluoromethyl)phenyl]carbamoyl | H | — | NMR |
| 13-215 | [4- | H | — | NMR |
| | (trifluoromethyl)phenyl]carbamoyl | | | |
| 13-216 | [3- | H | — | NMR |
| | (trifluoromethyl)phenyl]carbamoyl | | | |
| 13-217 | phenylcarbamoyl | H | — | NMR |
| 13-218 | (4- | H | — | NMR |
| | chlorophenyl)carbamoyl | | | |
| 13-219 | (4- | H | — | NMR |
| | methylphenyl)carbamoyl | | | |
| 13-220 | H | COCF₃ | — | logP (acid) 3.52 |
| 13-221 | [3,4- | H | — | NMR |
| | bis(trifluoromethyl)phenyl]carbamoyl | | | |
| 13-222 | (4- | H | — | NMR |
| | methoxyphenyl)carbamoyl | | | |
| 13-223 | 3-(trifluoromethyl)- | H | — | NMR |
| | 1,2,4-triazol-1-yl | | | |
| 13-224 | 4-chloro-pyrazol-1-yl | H | — | NMR |
| 13-225 | 3-(trifluoromethyl)- | H | — | NMR |
| | 1,2,4-triazol-1-yl | | | |
| 13-226 | C₂F₅ | CF₃ | — | logP (acid) 3.64 |
| 13-227 | CF₃ | SCF₃ | — | logP (acid) 5.09 |
| 13-228 | H | CF₃ | — | logP (acid) 2.98 |
| 13-229 | C₂F₅ | H | — | logP (acid) 3.4 |
| 13-230 | C₂F₅ | H | — | logP (acid) 3.29 |
| 13-231 | C₂F₅ | H | — | logP (acid) 2.76 |
| 13-232 | H | CF₃ | — | logP (acid) 3.97 |
| 13-233 | H | CF₃ | — | NMR |
| 13-234 | H | CF₃ | — | NMR |
| 13-235 | H | CF₃ | — | NMR |
| 13-236 | H | CF₃ | — | NMR |
| 13-237 | H | CF₃ | — | NMR |
| 13-238 | H | CF₃ | — | logP (acid) 3.74 |
| 13-239 | H | C₂F₅ | — | logP (acid) 4.27 |
| 13-240 | I | H | — | NMR |
| 13-241 | I | H | — | |
| 13-242 | I | H | — | |
| 13-243 | H | CF₃ | — | NMR |
| 13-244 | H | C₂F₅ | — | NMR |
| 13-245 | H | CF₃ | — | NMR |
| 13-246 | H | CF₃ | — | NMR |
| 13-247 | H | C₂F₅ | — | logP (acid) 2.85 |
| 13-248 | C₂F₅ | CF₃ | — | logP (acid) 2.87 |
| 13-249 | H | CF₃ | — | logP (acid) 2.17 |
| 13-250 | H | CF₃ | — | logP (acid) 2.64 |
| 13-251 | H | CF₃ | — | logP (acid) 4.45 |
| 13-252 | H | CF₃ | — | logP (acid) 3.47 |
| 13-253 | H | CF₃ | — | logP (acid) 3.28 |
| 13-254 | H | CF₃ | — | logP (acid) 3.19 |
| 13-255 | H | CF₃ | — | logP (acid) 2.80 |
| 13-256 | H | CF₃ | — | logP (acid) 4.46 |
| 13-257 | H | CF₃ | — | logP (acid) 4.41 |
| 13-258 | H | CF₃ | — | logP (acid) 4.13 |
| 13-259 | H | CF₃ | — | logP (acid) 4.27 |
| 13-260 | H | CF₃ | — | logP (acid) 4.4 |
| 13-261 | H | CF₃ | — | logP (acid) 2.80 |
| 13-262 | H | CF₃ | — | logP (acid) 2.80 |
| 13-263 | H | CF₃ | — | logP (acid) 2.80 |
| 13-264 | H | CF₃ | — | logP (acid) 2.36 |
| 13-265 | H | CF₃ | — | logP (acid) 1.87 |
| 13-266 | H | CF₃ | — | logP (acid) 2.4 |
| 13-267 | H | CF₃ | — | logP (acid) 3.02 |
| 13-268 | H | CF₃ | — | logP (acid) 4.45 |
| 13-269 | H | CF₃ | — | logP (acid) 3.78 |
| 13-270 | H | CF₃ | — | logP (acid) 2.84 |
| 13-271 | H | CF₃ | — | logP (acid) 3.37 |
| 13-272 | H | CF₃ | — | logP (acid) 2.01 |
| 13-273 | H | CF₃ | — | NMR |
| 13-274 | H | CF₃ | — | logP (acid) 2.50 |
| 13-275 | H | CF₃ | — | logP (acid) 3.58 |
| 13-276 | H | CF₃ | — | logP (acid) 3.28 |
| 13-277 | H | CF₃ | — | logP (acid) 3.42 |
| 13-278 | H | CF₃ | — | |
| 13-279 | H | CF₃ | — | logP (acid) 2.73 |
| 13-280 | H | CF₃ | — | |
| 13-281 | H | CF₃ | — | logP (acid) 4.41 |
| 13-282 | H | CF₃ | — | |
| 13-283 | H | CF₃ | — | logP (acid) 2.4 |
| 13-284 | H | CF₃ | — | mp 117-120° C. |
| 13-285 | H | CF₃ | — | logP (acid) 2.53 |
| 13-286 | H | CF₃ | — | logP (acid) 3.21 |
| 13-287 | H | CF₃ | — | logP (neutral) |
| | | | | 3.42 |
| 13-288 | H | CF₃ | — | logP (acid) 2.37 |
| 13-289 | H | CF₃ | — | logP (acid) 2.75 |
| 13-290 | C₂F₅ | H | — | logP (acid) 3.80 |
| 13-291 | H | CF₃ | — | NMR |
| 13-292 | C₂F₅ | H | — | mp 181-183° C. |
| 13-293 | C₂F₅ | H | — | mp 78-80° C. |
| 13-294 | C₂F₅ | H | — | NMR |
| 13-295 | H | CF₃ | — | NMR |
| 13-296 | H | CF₃ | — | NMR |
| 13-297 | H | CF₃ | — | NMR |
| 13-298 | H | CF₃ | — | mp 74-76° C. |
| 13-299 | H | OCHF₂ | — | NMR |
| 13-300 | H | OCHF₂ | — | NMR |
| 13-301 | H | CF₃ | — | NMR |
| 13-302 | H | CF₃ | — | NMR |
| 13-303 | I | H | — | NMR |
| 13-304 | I | H | — | |
| 13-305 | H | CF₃ | — | |
| 13-306 | H | CF₃ | — | NMR |
| 13-307 | CF₃ | C₂F₅ | — | NMR |
| 13-308 | CF₃ | F | — | logP (acid) 4.43 |
| 13-309 | CF₃ | F | — | |
| 13-310 | H | CF₃ | — | logP (acid) 4.74 |
| 13-311 | H | CF₃ | — | logP (acid) 4.20 |
| 13-312 | H | CF₃ | — | NMR |
| 13-313 | H | CF₃ | — | NMR |
| 13-314 | H | CF₃ | — | NMR |
| 13-315 | H | CF₃ | — | NMR |
| 13-316 | H | CF₃ | — | NMR |
| 13-317 | H | CF₃ | — | NMR |
| 13-318 | H | CF₃ | — | mp 141-143° C. |
| 13-319 | H | CF₃ | — | NMR |
| 13-320 | H | CF₃ | — | NMR |
| 13-321 | H | CF₃ | — | NMR |
| 13-322 | H | CF₃ | — | NMR |
| 13-323 | H | CF₃ | — | mp 192-194° C. |
| 13-324 | H | CF₃ | — | mp 87-88° C. |
| 13-325 | H | COCF₃ | — | logP (acid) 3.50 |
| 13-326 | 3-(trifluoromethyl)- | H | — | NMR |
| | 1,2,4-triazol-1-yl | | | |
| 13-327 | 3-(trifluoromethyl)- | H | — | NMR |
| | 1,2,4-triazol-1-yl | | | |
| 13-328 | 3-(trifluoromethyl)- | H | — | NMR |
| | 1,2,4-triazol-1-yl | | | |
| 13-329 | 3-(trifluoromethyl)- | H | — | NMR |
| | 1,2,4-triazol-1-yl | | | |
| 13-330 | 4-chloro-pyrazol-1- | H | — | NMR |
| | yl | | | |
| 13-331 | 4-chloro-pyrazol-1- | H | — | NMR |
| | yl | | | |
| 13-332 | phenylcarbamoyl | H | — | NMR |
| 13-333 | [3,4- | H | — | NMR |
| | bis(trifluoromethyl)phenyl]carbamoyl | | | |
| 13-334 | [3,5- | H | — | NMR |
| | bis(trifluoromethyl)phenyl]carbamoyl | | | |
| 13-335 | [3- | H | — | NMR |
| | (trifluoromethyl)phenyl]carbamoyl | | | |
| 13-336 | [4- | H | — | NMR |
| | (trifluoromethyl)phenyl]carbamoyl | | | |
| 13-337 | (4- | H | — | NMR |
| | methylphenyl)carbamoyl | | | |
| 13-338 | (4- | H | — | NMR |
| | chlorophenyl)carbamoyl | | | |
| 13-339 | (4- | H | — | NMR |
| | methoxyphenyl)carbamoyl | | | |
| 13-340 | C₂F₅ | H | — | logP (acid) 2.77 |
| 13-341 | C₂F₅ | H | — | logP (acid) 2.02 |
| 13-342 | H | C₂F₅ | — | logP (acid) 4.45 |
| 13-343 | H | CF₃ | — | logP (acid) 2.78 |
| 13-344 | H | CF₃ | — | NMR |
| 13-345 | H | CF₃ | — | logP (acid) 3.10 |
| 13-346 | H | CF₃ | — | logP (acid) 3.74 |
| 13-347 | H | CF₃ | — | NMR |
| 13-348 | H | CF₃ | — | NMR |
| 13-349 | H | CF₃ | — | logP (acid) 3.07 |
| 13-350 | H | H | — | NMR |
| 13-351 | CF₃ | — | — | |
| 13-352 | CF₃ | — | — | NMR |
| 13-353 | C₂F₅ | CF₃ | — | logP (acid) 4.19 |
| 13-354 | C₂F₅ | CF₃ | — | logP (acid) 2.91 |
| 13-355 | [3,4- | H | — | NMR |
| | bis(trifluoromethyl)phenyl]carbamoyl | | | |
| 13-356 | (2-cyano-3- | H | — | NMR |
| | methylbutan-2- | | | |
| | yl)carbamoyl | | | |
| 13-357 | [3,5- | H | — | NMR |
| | bis(trifluoromethyl)phenyl](methyl)carbamoyl | | | |
| 13-358 | [3,5- | H | — | NMR |
| | bis(trifluoromethyl)phenyl]carbamoyl | | | |
| 13-359 | [3- | H | — | |
| | (trifluoromethyl)phenyl]carbamoyl | | | |
| 13-360 | [3,4- | H | — | NMR |
| | bis(trifluoromethyl)phenyl]carbamoyl | | | |
| 13-361 | [3,4- | H | — | NMR |
| | bis(trifluoromethyl)phenyl]carbamoyl | | | |
| 13-362 | {[3-fluoro-4- | H | — | NMR |
| | (trifluoromethyl)phenyl]carbonyl}amino | | | |
| 13-363 | Cl | Cl | — | logP (acid) 2.17 |
| 13-364 | Cl | Cl | — | logP (acid) 2.59 |
| 13-365 | H | CF₃ | — | NMR |
| 13-366 | H | CF₃ | — | NMR |
| 13-367 | H | CF₃ | — | mp 175-176° C. |
| 13-368 | C₂F₅ | H | — | |
| 13-369 | C₂F₅ | H | — | |
| 13-370 | C₃F₇-n | H | — | |
| 13-371 | C₄F₉-n | H | — | |
| 13-372 | C₆F₁₃-n | H | — | |
| 13-373 | C₈F₁₇-n | H | — | |
| 13-374 | H | H | — | |
| 13-375 | H | CF₃ | — | |
| 13-376 | H | CF₃ | — | |
| 13-377 | H | CF₃ | — | |
| 13-378 | H | CF₃ | — | |
| 13-379 | H | CF₃ | — | |
| 13-380 | H | CF₃ | — | |
| 13-381 | H | CF₃ | — | |
| 13-382 | H | CF₃ | — | |
| 13-383 | H | CF₃ | — | |
| 13-384 | H | CF₃ | — | |
| 13-385 | H | CF₃ | — | |
| 13-386 | H | CF₃ | — | |
| 13-387 | H | CF₃ | — | |
| 13-388 | H | CF₃ | — | |
| 13-389 | H | CF₃ | — | |
| 13-390 | H | CF₃ | — | |
| 13-391 | H | CF₃ | — | |
| 13-392 | H | CF₃ | — | |
| 13-393 | H | CF₃ | — | |
| 13-394 | H | CF₃ | — | |
| 13-395 | H | CF₃ | — | |
| 13-396 | H | CF₃ | — | |
| 13-397 | H | CF₃ | — | |
| 13-398 | H | CF₃ | — | |
| 13-399 | H | CF₃ | — | |
| 13-400 | H | CF₃ | — | |
| 13-401 | H | CF₃ | — | |
| 13-402 | H | CF₃ | — | |
| 13-403 | H | CF₃ | — | |
| 13-404 | H | CF₃ | — | |
| 13-405 | H | CF₃ | — | |
| 13-406 | H | CF₃ | — | |
| 13-407 | H | CF₃ | — | |
| 13-408 | H | CF₃ | — | |
| 13-409 | H | CF₃ | — | |
| 13-410 | H | CF₃ | — | |
| 13-436 | H | CF₃ | — | |
| 13-437 | H | CF₃ | — | |
| 13-438 | H | CF₃ | — | |
| 13-439 | H | CF₃ | — | |
| 13-440 | H | CF₃ | — | |
| 13-441 | H | CF₃ | — | |
| 13-442 | H | CF₃ | — | |
| 13-443 | H | CF₃ | — | |
| 13-444 | H | CF₃ | — | |
| 13-445 | H | CF₃ | — | |
| 13-446 | H | CF₃ | — | |
| 13-447 | H | CF₃ | — | |
| 13-448 | H | CF₃ | — | |
| 13-449 | H | CF₃ | — | |
| 13-450 | H | CF₃ | — | |
| 13-451 | H | CF₃ | — | |
| 13-452 | H | CF₃ | — | |
| 13-453 | H | CF₃ | — | |
| 13-454 | H | CF₃ | — | |
| 13-455 | H | CF₃ | — | |
| 13-456 | H | CF₃ | — | |
| 13-457 | H | CF₃ | — | |
| 13-458 | H | CF₃ | — | |
| 13-459 | H | CF₃ | — | |
| 13-460 | H | CF₃ | — | |
| 13-461 | H | CF₃ | — | |
| 13-462 | H | CF₃ | — | |
| 13-463 | H | CF₃ | — | |
| 13-464 | H | CF₃ | — | |
| 13-465 | H | CF₃ | — | |
| 13-466 | H | CF₃ | — | |
| 13-467 | H | CF₃ | — | |
| 13-468 | H | CF₃ | — | |
| 13-469 | H | CF₃ | — | |
| 13-470 | H | CF₃ | — | |
| 13-471 | H | CF₃ | — | |
| 13-472 | H | CF₃ | — | |
| 13-473 | H | CF₃ | — | |
| 13-474 | H | CF₃ | — | |
| 13-475 | H | CF₃ | — | |
| 13-476 | H | CF₃ | — | |
| 13-477 | H | CF₃ | — | |
| 13-478 | H | CF₃ | — | |
| 13-479 | H | CF₃ | — | |
| 13-480 | H | CF₃ | — | |
| 13-481 | H | CF₃ | — | |
| 13-482 | H | CF₃ | — | |
| 13-483 | H | CF₃ | — | |
| 13-484 | H | CF₃ | — | |
| 13-485 | H | CF₃ | — | |
| 13-486 | H | CF₃ | — | |
| 13-487 | H | CF₃ | — | |
| 13-488 | H | CF₃ | — | |
| 13-489 | H | CF₃ | — | |
| 13-490 | H | CF₃ | — | |
| 13-491 | H | CF₃ | — | |
| 13-492 | H | CF₃ | — | |
| 13-493 | H | CF₃ | — | |
| 13-494 | H | CF₃ | — | |
| 13-495 | H | SCH₂CH₃ | — | |
| 13-496 | H | SOCH₂CH₃ | — | |
| 13-497 | H | SO₂CH₂CH₃ | — | |
| 13-498 | H | SCH₂CF₃ | — | |
| 13-499 | H | SOCH₂CF₃ | — | |
| 13-500 | H | SO₂CH₂CF₃ | — | |
| 13-501 | H | CONHCH₂CF₃ | — | NMR |
| 13-502 | Cl | Cl | — | logP (acid) |
| | | | | 3.50 |
| 13-503 | (3- | H | — | |
| | cyanophenyl)carbamoyl | | | |
| 13-504 | (3- | H | — | |
| | methylthiophenyl)carbamoyl | | | |
| 13-505 | [3- | H | — | |
| | (trifluoromethylthio)phenyl]carbamoyl | | | |
| 13-506 | H | CF₃ | — | |
| 13-507 | COCF3 | H | — | NMR |

TABLE 14

| No. | R¹ | R² | R³ | W¹ | W² | W³ |
|---|---|---|---|---|---|---|
| 14-1 | CH(CH₃)CH₂SO₂CH₃ (S)-isomer | H | H | CH | CH | CH |
| 14-2 | CH(CH₃)CH₂SCH₃ | H | H | CH | CH | CH |
| 14-3 | CH(CH₃)CH₂SO₂CH₃ | H | H | CH | CH | CH |
| 14-4 | CH(CH₃)CH₂SO₂CH₃ | H | H | CH | CH | CH |
| 14-5 | CH(CH₃)CH₂SCH₃ (S)-isomer | H | H | CH | CH | CH |
| 14-6 | CH(CH₃)CH₂SO₂CH₃ (S)-isomer | H | H | CH | CH | CH |
| 14-7 | CH(CH₃)CH₂SCH₃ (S)-isomer | H | H | CH | CH | CH |
| 14-8 | CH(CH₃)CH₂SC₂H5 (S)-isomer | H | H | CH | CH | CH |
| 14-9 | CH(CH₃)CH₂SC₂H5 (S)-isomer | H | H | CH | CH | CH |
| 14-10 | CH(CH₃)CH₂SC₂H5 (S)-isomer | H | H | CH | CH | CH |
| 14-11 | CH(CH₃)CH₂SO₂C₂H₅ (S)-isomer | H | H | CH | CH | CH |
| 14-12 | CH(CH₃)CH₂SO₂C₂H₅ (S)-isomer | H | H | CH | CH | CH |
| 14-13 | CH(CH₃)CH₂SCH₃ (S)-isomer | H | H | C—CH3 | CH | CH |
| 14-14 | CH(CH₃)CH₂SCH₃ (S)-isomer | H | H | C—CH₃ | CH | CH |
| 14-15 | CH(CH₃)CH₂SCH₃ (S)-isomer | H | H | C—C₂H₅ | CH | CH |
| 14-21 | CH(CH₃)CH₂SCH₃ (S)-isomer | H | H | C—Cl | CH | CH |
| 14-22 | CH(CH₃)CH₂SCH₃ (S)-isomer | H | H | C—Cl | CH | CH |
| 14-23 | CH(CH₃)CH₂SCH₃ (S)-isomer | H | H | C—Cl | CH | CH |
| 14-24 | CH(CH₃)CH₂SCH₃ (S)-isomer | H | H | C—Cl | CH | CH |
| 14-25 | CH(CH₃)CH₂SCH₃ (S)-isomer | H | H | C—Cl | CH | CH |
| 14-26 | CH(CH₃)CH₂SCH₃ (S)-isomer | H | H | C—Cl | CH | CH |
| 14-27 | CH(CH₃)CH₂SCH₃ (S)-isomer | H | H | C—Cl | CH | CH |
| 14-28 | CH(CH₃)CH₂SCH₃ (S)-isomer | H | H | C—Cl | CH | CH |
| 14-29 | CH(CH₃)CH₂SCH₃ (S)-isomer | H | H | C—Cl | CH | CH |
| 14-30 | CH(CH₃)CH₂SCH₃ (S)-isomer | H | H | C—Cl | CH | CH |
| 14-31 | CH(CH₃)CH₂SCH₃ (S)-isomer | H | H | C—Cl | CH | CH |
| 14-32 | CH(CH₃)CH₂SCH₃ (S)-isomer | H | H | C—Cl | CH | CH |
| 14-33 | CH(CH₃)CH₂SOCH₃ (S)-isomer | H | H | C—Cl | CH | CH |
| 14-34 | CH(CH₃)CH₂SO₂CH₃ (S)-isomer | H | H | C—Cl | CH | CH |
| 14-35 | CH(CH₃)CH₂SCH₃ (S)-isomer | H | H | C—Cl | CH | CH |
| 14-36 | CH(CH₃)CH₂SCH₃ (S)-isomer | H | H | C—Cl | CH | CH |
| 14-37 | CH(CH₃)CH₂SCH₃ (S)-isomer | H | H | C—Cl | CH | CH |
| 14-38 | CH(CH₃)CH₂SCH₃ (S)-isomer | H | H | C—Cl | CH | CH |
| 14-39 | CH(CH₃)CH₂SCH₃ (S)-isomer | H | H | C—Cl | CH | CH |
| 14-40 | CH(CH₃)CH₂SCH₃ (S)-isomer | H | H | C—Cl | CH | CH |
| 14-42 | CH(CH₃)CH₂SCH₃ (S)-isomer | H | H | C—Cl | CH | CH |
| 14-43 | CH(CH₃)CH₂SCH₃ (S)-isomer | H | H | C—Cl | CH | CH |
| 14-44 | CH(CH₃)CH₂SCH₃ (S)-isomer | H | H | C—Cl | CH | CH |
| 14-45 | CH(CH₃)CH₂SCH₃ (S)-isomer | H | H | C—Cl | CH | CH |
| 14-46 | CH(CH₃)CH₂SCH₃ (S)-isomer | H | H | C—Cl | CH | CH |
| 14-47 | CH(CH₃)CH₂SCH₃ (S)-isomer | H | H | C—Cl | CH | CH |
| 14-48 | CH(CH₃)CH₂SCH₃ (S)-isomer | H | H | C—Cl | CH | CH |
| 14-49 | CH(CH₃)CH₂SCH₃ (S)-isomer | H | H | C—Cl | CH | CH |
| 14-50 | CH(CH₃)CH₂SCH₃ (S)-isomer | H | H | C—Cl | CH | CH |
| 14-51 | CH(CH₃)CH₂SCH₃ (S)-isomer | H | H | C—Cl | CH | CH |
| 14-52 | CH(CH₃)CH₂SCH₃ (S)-isomer | H | H | C—Cl | CH | CH |
| 14-53 | CH(CH₃)CH₂SOCH₃ (S)-isomer | H | H | C—Cl | CH | CH |
| 14-54 | CH(CH₃)CH₂SOCH₃ (S)-isomer | H | H | C—Cl | CH | CH |
| 14-55 | CH(CH₃)CH₂SOCH₃ (S)-isomer | H | H | C—Cl | CH | CH |
| 14-56 | CH(CH₃)CH₂SOCH₃ (S)-isomer | H | H | C—Cl | CH | CH |
| 14-57 | CH(CH₃)CH₂SOCH₃ (S)-isomer | H | H | C—Cl | CH | CH |
| 14-58 | CH(CH₃)CH₂SOCH₃ (S)-isomer | H | H | C—Cl | CH | CH |
| 14-59 | CH(CH₃)CH₂SOCH₃ (S)-isomer | H | H | C—Cl | CH | CH |
| 14-60 | CH(CH₃)CH₂SOCH₃ (S)-isomer | H | H | C—Cl | CH | CH |
| 14-61 | CH(CH₃)CH₂SO₂CH₃ (S)-isomer | H | H | C—Cl | CH | CH |
| 14-62 | CH(CH₃)CH₂SO₂CH₃ (S)-isomer | H | H | C—Cl | CH | CH |
| 14-63 | CH(CH₃)CH₂SO₂CH₃ (S)-isomer | H | H | C—Cl | CH | CH |
| 14-64 | CH(CH₃)CH₂SO₂CH₃ (S)-isomer | H | H | C—Cl | CH | CH |
| 14-65 | CH(CH₃)CH₂SO₂CH₃ (S)-isomer | H | H | C—Cl | CH | CH |
| 14-66 | CH(CH₃)CH₂SO₂CH₃ (S)-isomer | H | H | C—Cl | CH | CH |
| 14-67 | CH(CH₃)CH₂SO₂CH₃ (S)-isomer | H | H | C—Cl | CH | CH |
| 14-68 | CH(CH₃)CH₂SO₂CH₃ (S)-isomer | H | H | C—Cl | CH | CH |
| 14-69 | CH(CH₃)CH₂SC₂H₅ (S)-isomer | H | H | C—Cl | CH | CH |
| 14-70 | CH(CH₃)CH₂SC₂H₅ (S)-isomer | H | H | C—Cl | CH | CH |
| 14-71 | CH(CH₃)CH₂SC₂H₅ (S)-isomer | H | H | C—Cl | CH | CH |
| 14-72 | CH(CH₃)CH₂SC₂H₅ (S)-isomer | H | H | C—Cl | CH | CH |
| 14-73 | CH(CH₃)CH₂SC₂H₅ (S)-isomer | H | H | C—Cl | CH | CH |
| 14-74 | CH(CH₃)CH₂SC₂H₅ (S)-isomer | H | H | C—Cl | CH | CH |
| 14-75 | CH(CH₃)CH₂SOC₂H₅ (S)-isomer | H | H | C—Cl | CH | CH |
| 14-76 | CH(CH₃)CH₂SOC₂H₅ (S)-isomer | H | H | C—Cl | CH | CH |
| 14-77 | CH(CH₃)CH₂SOC₂H₅ (S)-isomer | H | H | C—Cl | CH | CH |
| 14-78 | CH(CH₃)CH₂SO₂C₂H₅ (S)-isomer | H | H | C—Cl | CH | CH |
| 14-79 | CH(CH₃)CH₂SO₂C₂H₅ (S)-isomer | H | H | C—Cl | CH | CH |
| 14-80 | CH(CH₃)CH₂SO₂C₂H₅ (S)-isomer | H | H | C—Cl | CH | CH |
| 14-81 | C(CH₃)₂CH₂SCH₃ | H | H | C—Cl | CH | CH |
| 14-82 | CH(CH₃)CH₂SCH₃ (S)-isomer | H | H | C—Cl | CH | CH |
| 14-83 | CH(CH₃)CH₂SOCH₃ (S)-isomer | H | H | C—Cl | CH | CH |
| 14-84 | CH(CH₃)CH₂SO₂CH₃ (S)-isomer | H | H | C—Cl | CH | CH |
| 14-85 | CH(CH₃)CH₂SCH₃ (S)-isomer | H | H | C—Br | CH | CH |
| 14-86 | CH(CH₃)CH₂SOCH₃ (S)-isomer | H | H | C—Br | CH | CH |
| 14-87 | CH(CH₃)CH₂SO₂CH₃ (S)-isomer | H | H | C—Br | CH | CH |
| 14-88 | CH(CH₃)CH₂SCH₃ (S)-isomer | H | H | C—I | CH | CH |
| 14-89 | CH(CH₃)CH₂SOCH₃ (S)-isomer | H | H | C—I | CH | CH |
| 14-90 | CH(CH₃)CH₂SO₂CH₃ (S)-isomer | H | H | C—I | CH | CH |
| 14-91 | CH(CH₃)CH₂SCH₃ (S)-isomer | H | H | C—SCH₃ | CH | CH |
| 14-92 | CH(CH₃)CH₂SOCH₃ (S)-isomer | H | H | C—SCH₃ | CH | CH |
| 14-93 | CH(CH₃)CH₂SO₂CH₃ (S)-isomer | H | H | C—SCH₃ | CH | CH |
| 14-94 | CH(CH₃)CH₂SCH₃ (S)-isomer | H | H | C—SO₂CH₃ | CH | CH |
| 14-95 | CH(CH₃)CH₂SOCH₃ (S)-isomer | H | H | C—SO₂CH₃ | CH | CH |
| 14-96 | CH(CH₃)CH₂SO₂CH₃ (S)-isomer | H | H | C—SO₂CH₃ | CH | CH |
| 14-97 | CH(CH₃)CH₂SCH₃ (S)-isomer | H | H | C—Cl | CH | CH |
| 14-98 | CH(CH₃)CH₂SOCH₃ (S)-isomer | H | H | C—Cl | CH | CH |
| 14-99 | CH(CH₃)CH₂SO₂CH₃ (S)-isomer | H | H | C—Cl | CH | CH |
| 14-100 | CH(CH₃)CH₂SCH₃ (S)-isomer | H | H | C—Br | CH | CH |
| 14-101 | CH(CH₃)CH₂SOCH₃ (S)-isomer | H | H | C—Br | CH | CH |
| 14-102 | CH(CH₃)CH₂SO₂CH₃ (S)-isomer | H | H | C—Br | CH | CH |
| 14-103 | CH(CH₃)CH₂SCH₃ (S)-isomer | H | H | C—I | CH | CH |
| 14-104 | CH(CH₃)CH₂SOCH₃ (S)-isomer | H | H | C—I | CH | CH |
| 14-105 | CH(CH₃)CH₂SO₂CH₃ (S)-isomer | H | H | C—I | CH | CH |
| 14-106 | CH(CH₃)CH₂SCH₃ (S)-isomer | H | H | C—SCH₃ | CH | CH |
| 14-107 | CH(CH₃)CH₂SOCH₃ (S)-isomer | H | H | C—SCH₃ | CH | CH |
| 14-108 | CH(CH₃)CH₂SO₂CH₃ (S)-isomer | H | H | C—SCH₃ | CH | CH |
| 14-109 | CH(CH₃)CH₂SCH₃ (S)-isomer | H | H | C—SO₂CH₃ | CH | CH |
| 14-110 | CH(CH₃)CH₂SOCH₃ (S)-isomer | H | H | C—SO₂CH₃ | CH | CH |
| 14-111 | CH(CH₃)CH₂SO₂CH₃ (S)-isomer | H | H | C—SO₂CH₃ | CH | CH |
| 14-112 | C(CH₃)₂CH₂SCH₃ | H | H | C—Cl | CH | CH |
| 14-113 | C(CH₃)₂CH₂SCH₃ | H | H | C—Cl | CH | CH |
| 14-114 | C(CH₃)₂CH₂SCH₃ | H | H | C—Cl | CH | CH |
| 14-115 | C(CH₃)₂CH₂SOCH₃ | H | H | C—Cl | CH | CH |
| 14-116 | C(CH₃)₂CH₂SO₂CH₃ | H | H | C—Cl | CH | CH |
| 14-117 | C(CH₃)₂CH₂SC₂H₅ | H | H | C—Cl | CH | CH |
| 14-118 | C(CH₃)₂CH₂SC₂H₅ | H | H | C—Cl | CH | CH |
| 14-119 | C(CH₃)₂CH₂SC₂H₅ | H | H | C—Cl | CH | CH |
| 14-120 | C(CH₃)₂CH₂SC₂H₅ | H | H | C—Cl | CH | CH |
| 14-121 | C(CH3)2CH2SOC2H5 | H | H | C—Cl | CH | CH |
| 14-122 | C(CH₃)₂CH₂SOC₂H₅ | H | H | C—Cl | CH | CH |
| 14-123 | C(CH₃)₂CH₂SOC₂H₅ | H | H | C—Cl | CH | CH |
| 14-124 | C(CH₃)₂CH₂SOC₂H₅ | H | H | C—Cl | CH | CH |
| 14-125 | C(CH₃)₂CH₂SO₂C₂H₅ | H | H | C—Cl | CH | CH |
| 14-126 | C(CH₃)₂CH₂SO₂C₂H₅ | H | H | C—Cl | CH | CH |
| 14-127 | C(CH₃)₂CH₂SO₂C₂H₅ | H | H | C—Cl | CH | CH |
| 14-128 | C(CH₃)₂CH₂SO₂C₂H₅ | H | H | C—Cl | CH | CH |
| 14-129 | C(CH₃)₂CH₂OH | H | H | C—Cl | CH | CH |
| 14-130 | C(CH₃)₂CHO | H | H | C—Cl | CH | CH |
| 14-131 | C(CH₃)₂CO₂H | H | H | C—Cl | CH | CH |
| 14-132 | C(CH₃)₂CH═NOH | H | H | C—Cl | CH | CH |
| 14-133 | C(CH₃)₂CH₂OCH₂C₆H₅ | H | H | C—Cl | CH | CH |
| 14-134 | CH(CH₃)CH₂OCONHCH₃ (S)-isomer | H | H | C—Cl | CH | CH |
| 14-135 | CH(CH₃)CH₂OCONHC₂H₅ (S)-isomer | H | H | C—Cl | CH | CH |
| 14-136 | CH(CH₃)CH₂OCONHC₂H₅ (S)-isomer | H | H | C—Cl | CH | CH |
| 14-137 | CH(CH₃)CO₂CH₂C₆H₅ | H | H | C—Cl | CH | CH |
| 14-138 | CH(CH₃)CO₂CH₂C₆H₅ | H | H | C—Cl | CH | CH |
| 14-139 | C(CH₃)₂CO₂CH₂C₆H₅ | H | H | C—Cl | CH | CH |
| 14-140 | CH(CH₃)CONHCH₂CO₂CH₃ | H | H | C—Cl | CH | CH |
| 14-141 | CH(CH₃)CONHCH₂CO₂CH₃ | H | H | C—Cl | CH | CH |
| 14-142 | C(CH₃)₂CONHCH₂CO₂CH₃ | H | H | C—Cl | CH | CH |
| 14-143 | C(CH₃)₂CONH-cyclopropyl | H | H | C—Cl | CH | CH |
| 14-144 | C(CH₃)₂CONHCH₂C≡CH | H | H | C—Cl | CH | CH |
| 14-145 | CH(CH₃)CH₂SCH₃ (S)-isomer | H | H | C—Cl | CH | CH |
| 14-146 | CH(CH₃)CH₂SCH₃ (S)-isomer | H | H | C—Cl | CH | CH |
| 14-147 | CH(CH₃)CH₂SOCH₃ (S)-isomer | H | H | C—Cl | CH | CH |
| 14-148 | CH(CH₃)CH₂SCH₃ (S)-isomer | H | H | C—Cl | CH | CH |
| 14-149 | CH(CH₃)CH₂SCH₃ (S)-isomer | H | H | C—Cl | CH | CH |
| 14-150 | CH(CH₃)CH₂SCH₃ (S)-isomer | H | H | C—Cl | CH | CH |
| 14-151 | CH(CH₃)CH₂SCH₃ (S)-isomer | H | H | C—Cl | CH | CH |
| 14-152 | CH(CH₃)CH₂SCH₃ (S)-isomer | H | H | C—Cl | CH | CH |
| 14-153 | CH(CH₃)CH₂SOCH₃ (S)-isomer | H | H | C—Cl | CH | CH |
| 14-154 | CH(CH₃)CH₂SOCH₃ (S)-isomer | H | H | C—Cl | CH | CH |
| 14-155 | CH(CH₃)CH₂SO₂CH₃ (S)-isomer | H | H | C—Cl | CH | CH |
| 14-156 | CH(CH₃)CH₂SO₂CH₃ (S)-isomer | H | H | C—Cl | CH | CH |
| 14-157 | CH(CH₃)CH₂SCH₃ (S)-isomer | H | H | C—Cl | CH | CH |
| 14-158 | CH(CH₃)CH₂SCH₃ (S)-isomer | H | H | C—Cl | CH | CH |
| 14-159 | CH(CH₃)CH₂SCH₃ (S)-isomer | H | H | C—Cl | CH | CH |
| 14-160 | CH(CH₃)CH₂SOCH₃ (S)-isomer | H | H | C—Cl | CH | CH |
| 14-161 | CH(CH₃)CH₂SOCH₃ (S)-isomer | H | H | C—Cl | CH | CH |
| 14-162 | CH(CH₃)CH₂SO₂CH₃ (S)-isomer | H | H | C—Cl | CH | CH |
| 14-163 | CH(CH₃)CH₂SCH₃ (S)-isomer | H | H | C—Cl | CH | CH |
| 14-164 | CH(CH₃)CH₂SCH₃ (S)-isomer | H | H | CH | CH | CH |
| 14-165 | CH(CH₃)CH₂SOCH₃ (S)-isomer | H | H | C—Cl | CH | CH |
| 14-166 | CH(CH₃)CH₂SO₂CH₃ (S)-isomer | H | H | C—Cl | CH | CH |
| 14-167 | CH(CH₃)CH₂SOCH₃ (S)-isomer | H | H | C—Cl | CH | CH |
| 14-168 | CH(CH₃)CH₂SO₂CH₃ (S)-isomer | H | H | C—Cl | CH | CH |
| 14-169 | CH(CH₃)CH₂SCH₃ (S)-isomer | H | H | C—Br | CH | CH |
| 14-170 | CH(CH₃)CH₂SCH₃ (S)-isomer | H | H | C—Br | CH | CH |
| 14-171 | CH(CH₃)CH₂SCH₃ (S)-isomer | H | H | C—Br | CH | CH |
| 14-172 | CH(CH₃)CH₂SCH₃ (S)-isomer | H | H | C—Br | CH | CH |
| 14-173 | CH(CH₃)CH₂SCH₃ (S)-isomer | H | H | C—Br | CH | CH |
| 14-174 | CH(CH₃)CH₂SOCH₃ (S)-isomer | H | H | C—Br | CH | CH |
| 14-175 | CH(CH₃)CH₂SO₂CH₃ (S)-isomer | H | H | C—Br | CH | CH |
| 14-176 | CH(CH₃)CH₂SC₂H5 (S)-isomer | H | H | C—Br | CH | CH |
| 14-177 | CH(CH₃)CH₂SOC₂H₅ (S)-isomer | H | H | C—Br | CH | CH |
| 14-178 | CH(CH₃)CH₂SO₂C₂H₅ (S)-isomer | H | H | C—Br | CH | CH |
| 14-179 | C(CH₃)₂CH₂SCH₃ | H | H | C—Br | CH | CH |
| 14-180 | C(CH₃)₂CH₂SOCH₃ | H | H | C—Br | CH | CH |
| 14-181 | C(CH₃)₂CH₂SO₂CH₃ | H | H | C—Br | CH | CH |
| 14-182 | C(CH₃)₂CH₂SC₂H₅ | H | H | C—Br | CH | CH |
| 14-183 | C(CH₃)₂CH₂SC₂H₅ | H | H | C—Br | CH | CH |
| 14-184 | C(CH₃)₂CH₂SC₂H₅ | H | H | C—Br | CH | CH |
| 14-185 | C(CH₃)₂CH₂SC₂H₅ | H | H | C—Br | CH | CH |
| 14-186 | C(CH₃)₂CH₂SOC₂H₅ | H | H | C—Br | CH | CH |
| 14-187 | C(CH₃)₂CH₂SOC₂H₅ | H | H | C—Br | CH | CH |
| 14-188 | C(CH₃)₂CH₂SOC₂H₅ | H | H | C—Br | CH | CH |
| 14-189 | C(CH₃)₂CH₂SOC₂H₅ | H | H | C—Br | CH | CH |
| 14-190 | C(CH₃)₂CH₂SO₂C₂H₅ | H | H | C—Br | CH | CH |
| 14-191 | C(CH₃)₂CH₂SO₂C₂H₅ | H | H | C—Br | CH | CH |
| 14-192 | C(CH₃)₂CH₂SO₂C₂H₅ | H | H | C—Br | CH | CH |
| 14-193 | C(CH₃)₂CH₂SO₂C₂H₅ | H | H | C—Br | CH | CH |
| 14-194 | CH(CH₃)CH₂SCH₃ (S)-isomer | H | H | C—I | CH | CH |
| 14-195 | C(CH₃)₂CH₂SCH₃ | H | H | C—I | CH | CH |
| 14-196 | CH(CH₃)₂ | H | H | C—I | CH | CH |
| 14-197 | CH(CH₃)CH₂SCH₃ (S)-isomer | H | H | C—I | CH | CH |
| 14-198 | CH(CH₃)CH₂SCH₃ (S)-isomer | H | H | C—I | CH | CH |
| 14-199 | CH(CH₃)CH₂SCH₃ (S)-isomer | H | H | C—I | CH | CH |
| 14-200 | CH(CH₃)CH₂SCH₃ (S)-isomer | H | H | C—I | CH | CH |
| 14-201 | CH(CH₃)CH₂SCH₃ (S)-isomer | H | H | C—I | CH | CH |
| 14-202 | CH(CH₃)CH₂SOCH₃ (S)-isomer | H | H | C—I | CH | CH |
| 14-203 | CH(CH₃)CH₂SOCH₃ (S)-isomer | H | H | C—I | CH | CH |
| 14-204 | CH(CH₃)CH₂SO₂CH₃ (S)-isomer | H | H | C—I | CH | CH |
| 14-205 | CH(CH₃)CH₂SO₂CH₃ (S)-isomer | H | H | C—I | CH | CH |
| 14-206 | CH(CH₃)CH₂SC₂H5 (S)-isomer | H | H | C—I | CH | CH |
| 14-207 | CH(CH₃)CH₂SOC₂H₅ (S)-isomer | H | H | C—I | CH | CH |
| 14-208 | CH(CH₃)CH₂SO₂C₂H₅ (S)-isomer | H | H | C—I | CH | CH |
| 14-209 | C(CH₃)₂CH₂SCH₃ | H | H | C—I | CH | CH |
| 14-210 | C(CH₃)₂CH₂SCH₃ | H | H | C—I | CH | CH |
| 14-211 | C(CH₃)₂CH₂SCH₃ | H | H | C—I | CH | CH |
| 14-212 | C(CH₃)₂CH₂SCH₃ | H | H | C—I | CH | CH |
| 14-213 | C(CH₃)₂CH₂SOCH₃ | H | H | C—I | CH | CH |
| 14-214 | C(CH₃)₂CH₂SO₂CH₃ | H | H | C—I | CH | CH |
| 14-215 | C(CH₃)₂CH₂SO₂CH₃ | H | H | C—I | CH | CH |
| 14-216 | C(CH₃)₂CH₂SC₂H₅ | H | H | C—I | CH | CH |
| 14-217 | C(CH₃)₂CH₂SC₂H₅ | H | H | C—I | CH | CH |
| 14-218 | C(CH₃)₂CH₂SC₂H₅ | H | H | C—I | CH | CH |
| 14-219 | C(CH₃)₂CH₂SC₂H₅ | H | H | C—I | CH | CH |
| 14-220 | C(CH₃)₂CH₂SOC₂H₅ | H | H | C—I | CH | CH |
| 14-221 | C(CH₃)₂CH₂SOC₂H₅ | H | H | C—I | CH | CH |
| 14-222 | C(CH₃)₂CH₂SOC₂H₅ | H | H | C—I | CH | CH |
| 14-223 | C(CH₃)₂CH₂SOC₂H₅ | H | H | C—I | CH | CH |
| 14-224 | C(CH₃)₂CH₂SO₂C₂H₅ | H | H | C—I | CH | CH |
| 14-225 | C(CH₃)₂CH₂SO₂C₂H₅ | H | H | C—I | CH | CH |
| 14-226 | C(CH₃)₂CH₂SO₂C₂H₅ | H | H | C—I | CH | CH |
| 14-227 | C(CH₃)₂CH₂SO₂C₂H₅ | H | H | C—I | CH | CH |
| 14-228 | C(CH₃)₂CH₂SCH₃ | H | H | C—I | CH | CH |
| 14-229 | C(CH₃)₂CH₂SCH₃ | H | H | C—I | CH | CH |
| 14-230 | C(CH₃)₂CH₂SCH₃ | H | H | C—I | CH | CH |
| 14-231 | C(CH₃)₂CH₂SO₂CH₃ | H | H | C—I | CH | CH |
| 14-232 | CH(CH₃)CH₂SCH₃ (S)-isomer | H | H | C—I | CH | CH |
| 14-233 | CH(CH₃)CH₂SCH₃ (S)-isomer | H | H | C—I | CH | CH |
| 14-234 | CH(CH₃)CH₂SOCH₃ (S)-isomer | H | H | C—I | CH | CH |
| 14-235 | CH(CH₃)CH₂SOCH₃ (S)-isomer | H | H | C—I | CH | CH |
| 14-236 | CH(CH₃)CH₂SCH₃ | H | H | C—I | CH | CH |
| 14-237 | CH(CH₃)CH₂SO₂CH₃ | H | H | C—I | CH | CH |
| 14-238 | CH(CH₃)CH₂SCH₃ | H | H | C—I | CH | CH |
| 14-239 | C(CH₃)₂CH₂SCH₃ | H | H | C—I | CH | CH |
| 14-240 | C(CH₃)₂CH₂SO₂CH₃ | H | H | C—I | CH | CH |
| 14-241 | C(CH₃)₂CH₂SO₂CH₃ | H | H | C—I | CH | CH |
| 14-242 | C(CH₃)₃ | H | H | C—I | CH | CH |
| 14-243 | CH₂CH₂SCH₃ | H | H | C—I | CH | CH |
| 14-244 | CH₂CH₂SC₂H₅ | H | H | C—I | CH | CH |
| 14-245 | CH₂CH(CH₃)SCH₃ | H | H | C—I | CH | CH |
| 14-246 | CH(CH₃)CH₂SCH₃ | H | H | C—I | CH | CH |
| 14-247 | CH(CH₃)CH₂SO₂CH₃ | H | H | C—I | CH | CH |
| 14-249 | CH(CH₃)CH₂SO₂CH₃ (S)-isomer | H | H | C—I | CH | CH |
| 14-250 | CH(CH₃)CH₂SCH₃ (R)-isomer | H | H | C—I | CH | CH |
| 14-251 | CH(CH₃)CH₂SO₂CH₃ (R)-isomer | H | H | C—I | CH | CH |
| 14-252 | CH(CH₃)CH₂SC₂H5 (S)-isomer | H | H | C—I | CH | CH |
| 14-253 | CH(CH₃)CH₂SC₂H5 (S)-isomer | H | H | C—I | CH | CH |
| 14-254 | CH(CH₃)CH₂SC₂H5 (S)-isomer | H | H | C—I | CH | CH |
| 14-255 | CH(CH₃)CH₂SO₂C₂H₅ (S)-isomer | H | H | C—I | CH | CH |
| 14-256 | CH(CH₃)CH₂SO₂C₂H₅ (S)-isomer | H | H | C—I | CH | CH |
| 14-257 | CH(CH₃)CH₂SO₂C₂H₅ (S)-isomer | H | H | C—I | CH | CH |
| 14-258 | CH(CH₃)CH₂SC₃H₇-n (S)-isomer | H | H | C—I | CH | CH |
| 14-259 | CH(CH₃)CH₂SO₂C₃H₇-n (S)-isomer | H | H | C—I | CH | CH |
| 14-260 | C(C₂H₅)₂CH₂SCH₃ | H | H | C—I | CH | CH |
| 14-261 | C(CH₃)₂C≡CH | H | H | C—I | CH | CH |
| 14-262 | CH(CH₃)CH₂SCH₃ (S)-isomer | H | H | C—CF₃ | CH | CH |
| 14-263 | CH(CH₃)CH₂SCH₃ (S)-isomer | H | H | C—CF₃ | CH | CH |
| 14-264 | CH(CH₃)CH₂SO₂CH₃ (S)-isomer | H | H | C-[3,5-bis(trifluoromethyl)phenyl] | CH | CH |
| 14-265 | CH(CH₃)CH₂SCH₃ (S)-isomer | H | H | C—NO₂ | CH | CH |
| 14-266 | CH(CH₃)CH₂SCH₃ (S)-isomer | H | H | C—NHCOCH₃ | CH | CH |
| 14-267 | CH(CH₃)CH₂SCH₃ (S)-isomer | H | H | CH | C—Cl | CH |
| 14-268 | CH(CH₃)CH₂SCH₃ (S)-isomer | H | H | C—Cl | CH | CH |
| 14-269 | CH(CH₃)CH₂SOCH₃ (S)-isomer | H | H | C—Cl | CH | CH |
| 14-270 | CH(CH₃)CH₂SO₂CH₃ (S)-isomer | H | H | C—Cl | CH | CH |
| 14-271 | CH(CH₃)CH₂SCH₃ (S)-isomer | H | H | C—Br | CH | CH |
| 14-272 | CH(CH₃)CH₂SOCH₃ (S)-isomer | H | H | C—Br | CH | CH |
| 14-273 | CH(CH₃)CH₂SO₂CH₃ (S)-isomer | H | H | C—Br | CH | CH |
| 14-274 | CH(CH₃)CH₂SCH₃ (S)-isomer | H | H | C—I | CH | CH |
| 14-275 | CH(CH₃)CH₂SOCH₃ (S)-isomer | H | H | C—I | CH | CH |
| 14-276 | CH(CH₃)CH₂SO₂CH₃ (S)-isomer | H | H | C—I | CH | CH |
| 14-277 | CH(CH₃)CH₂SCH₃ (S)-isomer | H | H | C—Cl | CH | CH |
| 14-278 | CH(CH₃)CH₂SOCH₃ (S)-isomer | H | H | C—Cl | CH | CH |
| 14-279 | CH(CH₃)CH₂SO₂CH₃ (S)-isomer | H | H | C—Cl | CH | CH |
| 14-280 | CH(CH₃)CH₂SCH₃ (S)-isomer | H | H | C—Br | CH | CH |
| 14-281 | CH(CH₃)CH₂SOCH₃ (S)-isomer | H | H | C—Br | CH | CH |
| 14-282 | CH(CH₃)CH₂SO₂CH₃ (S)-isomer | H | H | C—Br | CH | CH |
| 14-283 | CH(CH₃)CH₂SCH₃ (S)-isomer | H | H | C—I | CH | CH |
| 14-284 | CH(CH₃)CH₂SOCH₃ (S)-isomer | H | H | C—I | CH | CH |
| 14-285 | CH(CH₃)CH₂SO₂CH₃ (S)-isomer | H | H | C—I | CH | CH |
| 14-286 | CH(CH₃)CH₂SCH₃ (S)-isomer | H | H | C—Br | CH | CH |
| 14-287 | CH(CH₃)CH₂SCH₃ (S)-isomer | H | H | C—I | CH | CH |
| 14-288 | CH(CH₃)CH₂SOCH₃ (S)-isomer | H | H | C—Br | CH | CH |
| 14-289 | CH(CH₃)CH₂SOCH₃ (S)-isomer | H | H | C—I | CH | CH |
| 14-290 | CH(CH₃)CH₂SO₂CH₃ (S)-isomer | H | H | C—I | CH | CH |
| 14-291 | CH(CH₃)CH₂SO₂CH₃ (S)-isomer | H | H | C—Br | CH | CH |
| 14-292 | CH(CH₃)CH₂SCH₃ (S)-isomer | H | H | C—I | CH | CH |
| 14-293 | CH(CH₃)CH₂SCH₃ (S)-isomer | H | H | C—I | CH | CH |
| 14-294 | CH(CH₃)CH₂SCH₃ (S)-isomer | H | H | C—Cl | CH | CH |
| 14-295 | CH(CH₃)CH₂C(O)CH₃ | H | H | C—Cl | CH | CH |
| 14-296 | CH(CH₃)CH₂C(O)CH₃ | H | H | C—Br | CH | CH |
| 14-297 | CH(CH₃)CH₂C(O)CH₃ | H | H | C—I | CH | CH |
| 14-298 | CH(CH₃)CH₂OC(O)CH₃ | H | H | C—Cl | CH | CH |
| | (S)-isomer | | | | | |
| 14-299 | CH(CH₃)CH₂SCH₃ (S)-isomer | H | H | C—Br | CH | CH |
| 14-300 | CH(CH₃)CH₂SCH₃ (S)-isomer | H | H | C—Br | CH | N |
| 14-301 | CH₃ | H | H | C—NO₂ | CH | CH |
| 14-302 | CH(CH₃)CH₂SCH₃ (S)-isomer | H | H | C—Br | CH | CH |
| 14-303 | CH(CH₃)CH₂SCH₃ (S)-isomer | H | H | C—CH═CH₂ | CH | CH |
| 14-304 | CH(CH₃)CH₂SCH₃ (S)-isomer | H | H | C—Cl | CH | CH |
| 14-305 | CH(CH₃)CH₂SCH₃ (S)-isomer | H | H | C—Cl | CH | CH |
| 14-306 | CH(CH₃)CH₂SCH₃ (S)-isomer | H | H | C—Cl | CH | CH |
| 14-307 | CH(CH₃)CH₂SCH₃ (S)-isomer | H | H | C—Cl | CH | CH |
| 14-308 | CH(CH₃)CH₂SCH₃ (S)-isomer | H | H | C—Cl | CH | N |
| 14-309 | CH(CH₃)CH₂SCH₃ (S)-isomer | H | H | C—Cl | CH | CH |
| 14-310 | CH(CH₃)CH₂SCH₃ (S)-isomer | H | H | C—Cl | CH | CH |
| 14-311 | CH(CH₃)CH₂SCH₃ (S)-isomer | H | H | C—Cl | CH | CH |
| 14-312 | CH(CH₃)CH₂SCH₃ (S)-isomer | H | H | C—Cl | CH | CH |
| 14-313 | CH(CH₃)CH₂SCH₃ (S)-isomer | H | H | C—Cl | CH | CH |
| 14-314 | CH(CH₃)CH₂SCH₃ (S)-isomer | H | H | C—Cl | CH | CH |
| 14-315 | CH(CH₃)CH₂SCH₃ (S)-isomer | H | H | C—Cl | CH | CH |
| 14-316 | CH(CH₃)CH₂SCH₃ (S)-isomer | H | H | C—Cl | CH | CH |
| 14-317 | CH(CH₃)CH₂SCH₃ (S)-isomer | H | H | C—Cl | CH | CH |
| 14-318 | CH(CH₃)CH₂SOCH₃ (S)-isomer | H | H | C—Cl | CH | CH |
| 14-319 | CH(CH₃)CH₂SO₂CH₃ (S)-isomer | H | H | C—Cl | CH | CH |
| 14-320 | CH(CH₃)CH₂SCH₃ (S)-isomer | H | H | C—Cl | CH | CH |
| 14-321 | CH(CH₃)CH₂SOCH₃ (S)-isomer | H | H | C—Cl | CH | CH |
| 14-322 | CH(CH₃)CH₂SO₂CH₃ (S)-isomer | H | H | C—Cl | CH | CH |
| 14-323 | CH(CH₃)CH₂SCH₃ (S)-isomer | H | H | C—Cl | CH | CH |
| 14-324 | CH(CH₃)CH₂SCH₃ (S)-isomer | H | H | C—Cl | CH | CH |
| 14-325 | CH(CH₃)CH₂SCH₃ (S)-isomer | H | H | C—Br | CH | CH |
| 14-326 | CH(CH₃)CH₂SCH₃ (S)-isomer | H | H | C—SCH₃ | CH | CH |
| 14-327 | CH(CH₃)CH₂SCH₃ (S)-isomer | H | H | C—SO₂CH₃ | CH | CH |
| 14-328 | CH(CH₃)CH₂SCH₃ (S)-isomer | H | H | C—Cl | CH | CH |
| 14-329 | CH(CH₃)CH₂SOCH₃ (S)-isomer | H | H | C—Cl | CH | CH |
| 14-330 | CH(CH₃)CH₂SO₂CH₃ (S)-isomer | H | H | C—Cl | CH | CH |
| 14-331 | CH(CH₃)CH₂SCH₃ (S)-isomer | H | H | C—Br | CH | CH |
| 14-332 | CH(CH₃)CH₂SOCH₃ (S)-isomer | H | H | C—Br | CH | CH |
| 14-333 | CH(CH₃)CH₂SO₂CH₃ (S)-isomer | H | H | C—Br | CH | CH |
| 14-334 | CH(CH₃)CH₂SCH₃ (S)-isomer | H | H | C—I | CH | CH |
| 14-335 | CH(CH₃)CH₂SOCH₃ (S)-isomer | H | H | C—I | CH | CH |
| 14-336 | CH(CH₃)CH₂SO₂CH₃ (S)-isomer | H | H | C—I | CH | CH |
| 14-337 | CH(CH₃)CH₂SCH₃ (S)-isomer | H | H | C—Cl | CH | CH |
| 14-338 | CH(CH₃)CH₂SCH₃ (S)-isomer | H | H | C—Cl | CH | CH |
| 14-339 | CH(CH₃)CH₂SCH₃ (S)-isomer | H | H | C—Cl | CH | CH |
| 14-340 | CH(CH₃)CH₂SCH₃ (S)-isomer | H | H | C—Cl | CH | CH |
| 14-341 | CH(CH₃)CH₂SCH₃ (S)-isomer | H | H | CH | CH | CH |
| 14-342 | CH(CH₃)CH₂SCH₃ (S)-isomer | H | H | CH | CH | CH |
| 14-343 | CH(CH₃)CH₂SOCH₃ (S)-isomer | H | H | C—Cl | CH | CH |
| 14-344 | CH(CH₃)CH₂SOCH₃ (S)-isomer | H | H | C—Cl | CH | CH |
| 14-345 | CH(CH₃)CH₂SOCH₃ (S)-isomer | H | H | CH | CH | CH |
| 14-346 | CH(CH₃)CH₂SOCH₃ (S)-isomer | H | H | CH | CH | CH |
| 14-347 | CH(CH₃)CH₂SOCH₃ (S)-isomer | H | H | C—NO₂ | CH | CH |
| 14-348 | CH(CH₃)CH₂SO₂CH₃ (S)-isomer | H | H | C—Br | CH | CH |
| 14-349 | CH(CH₃)CH₂SO₂CH₃ (S)-isomer | H | H | C—Cl | CH | CH |
| 14-350 | CH(CH₃)CH₂SO₂CH₃ (S)-isomer | H | H | C—Cl | CH | CH |
| 14-351 | CH(CH₃)CH₂SO₂CH₃ (S)-isomer | H | H | C—Cl | CH | CH |
| 14-352 | CH(CH₃)CH₂SO₂CH₃ (S)-isomer | H | H | C—Cl | CH | CH |
| 14-353 | CH(CH₃)CH₂SCH₃ (S)-isomer | H | H | C—Cl | CH | CH |
| 14-354 | CH(CH₃)CH₂SO₂CH₃ (S)-isomer | H | H | C—Cl | CH | CH |
| 14-355 | CH(CH₃)CH₂SO₂CH₃ (S)-isomer | H | H | C—Cl | CH | CH |
| 14-356 | CH(CH₃)CH₂SO₂CH₃ (S)-isomer | H | H | C—NO₂ | CH | CH |
| 14-357 | R1a | H | H | C—Cl | CH | CH |
| 14-358 | R1a | H | H | C—I | CH | CH |
| 14-359 | R1a | H | H | C—Br | CH | CH |
| 14-360 | R1b | H | H | C—Cl | CH | CH |
| 14-361 | R1b | H | H | C—I | CH | CH |
| 14-362 | R1b | H | H | C—Br | CH | CH |
| 14-363 | R1q | H | H | C—Br | CH | CH |
| 14-364 | CH(CH₃)CH₂SCH₃ (S)-isomer | H | H | C—SO₂CH₃ | CH | CH |
| 14-365 | CH(CH₃)CH₂SCH₃ (S)-isomer | H | H | C—SO₂N(CH₃)₂ | CH | CH |
| 14-366 | CH(CH₃)CH₂SOCH₃ (S)-isomer | H | H | C—SO₂CH₃ | CH | CH |
| 14-367 | CH(CH₃)CH₂SOCH₃ (S)-isomer | H | H | C—SO₂N(CH₃)₂ | CH | CH |
| 14-368 | CH(CH₃)CH₂SO₂CH₃ (S)-isomer | H | H | C—SO₂CH₃ | CH | CH |
| 14-369 | CH(CH₃)CH₂SO₂CH₃ (S)-isomer | H | H | C—SO₂N(CH₃)₂ | CH | CH |
| 14-370 | CH(CH₃)CH₂SCH₃ (S)-isomer | H | H | C—SCH₃ | CH | CH |
| 14-371 | CH(CH₃)CH₂SOCH₃ (S)-isomer | H | H | C—SCH₃ | CH | CH |
| 14-372 | CH(CH₃)CH₂SCH₃ (S)-isomer | H | H | C—SO₂CH₃ | CH | CH |
| 14-373 | CH(CH₃)CH₂SOCH₃ (S)-isomer | H | H | C—SOCH₃ | CH | CH |
| 14-374 | CH(CH₃)CH₂SOCH₃ (S)-isomer | H | H | C—SO₂CH₃ | CH | CH |
| 14-375 | CH(CH₃)CH₂SO₂CH₃ (S)-isomer | H | H | C—SO₂CH₃ | CH | CH |
| 14-376 | CH(CH₃)CH₂SCH₃ (S)-isomer | H | H | C—SCH₃ | CH | CH |
| 14-377 | CH(CH₃)CH₂SCH₃ (S)-isomer | H | H | C—SO₂CH₃ | CH | CH |
| 14-378 | CH(CH₃)CH₂SCH₃ (S)-isomer | H | H | C—SCH₃ | CH | CH |
| 14-379 | CH(CH₃)CH₂SCH₃ (S)-isomer | H | H | C—SO₂CH₃ | CH | CH |
| 14-380 | CH(CH₃)CH₂SCH₃ (S)-isomer | H | H | C—SCH₃ | CH | CH |
| 14-381 | CH(CH₃)CH₂SCH₃ (S)-isomer | H | H | C—SO₂CH₃ | CH | CH |
| 14-382 | CH(CH₃)CH₂SCH₃ (S)-isomer | H | H | C—SC₂H₅ | CH | CH |
| 14-383 | CH(CH₃)CH₂SCH₃ (S)-isomer | H | H | C—SCH₃ | CH | CH |
| 14-384 | CH(CH₃)CH₂SCH₃ (S)-isomer | H | H | C—SO₂N(CH₃)₂ | CH | CH |
| 14-385 | CH(CH₃)CH₂SCH₃ (S)-isomer | H | H | C—SCH₃ | CH | CH |
| 14-386 | CH(CH₃)CH₂SCH₃ (S)-isomer | H | H | C—SCH₃ | CH | CH |
| 14-387 | CH(CH₃)CH₂SCH₃ (S)-isomer | H | H | C—SCH₃ | CH | CH |
| 14-388 | CH(CH₃)CH₂SCH₃ (S)-isomer | H | H | C—SO₂CH₃ | CH | CH |
| 14-389 | CH(CH₃)CH₂SCH₃ (S)-isomer | H | H | C—SCH₃ | CH | CH |
| 14-390 | CH(CH₃)CH₂SCH₃ (S)-isomer | H | H | C—SO₂CH₃ | CH | CH |
| 14-391 | CH(CH₃)CH₂SCH₃ (S)-isomer | H | H | C—SO₂C₂H₅ | CH | CH |
| 14-392 | CH(CH₃)CH₂SOCH₃ (S)-isomer | H | H | C—SO₂CH₃ | CH | CH |
| 14-393 | CH(CH₃)CH₂SCH₃ (S)-isomer | H | H | C—SO₂CH₃ | CH | CH |
| 14-394 | CH(CH₃)CH₂SOCH₃ (S)-isomer | H | H | C—SO₂CH₃ | CH | CH |
| 14-395 | CH(CH₃)CH₂SO₂CH₃ (S)-isomer | H | H | C—SO₂N(CH₃)₂ | CH | CH |
| 14-396 | CH(CH₃)CH₂SO₂CH₃ (S)-isomer | H | H | C—SO₂CH₃ | CH | CH |
| 14-397 | CH(CH₃)CH₂SCH₃ (S)-isomer | H | H | C—SCH₃ | CH | CH |
| 14-398 | CH(CH₃)CH₂SCH₃ (S)-isomer | H | H | CH | CH | CH |
| 14-399 | CH(CH₃)CH₂SCH₃ (S)-isomer | H | H | CH | CH | CH |
| 14-400 | CH(CH₃)CH₂SCH₃ (S)-isomer | H | H | CH | CH | CH |
| 14-401 | CH(CH₃)CH₂SCH₃ (S)-isomer | H | H | C—SCH₃ | CH | CH |
| 14-402 | CH(CH₃)CH₂SCH₃ (S)-isomer | H | H | C—SCH(CH₃)₂ | CH | CH |
| 14-403 | CH(CH₃)CH₂SCH₃ (S)-isomer | H | H | C—SCH₂-cyclopropyl | CH | CH |
| 14-404 | CH(CH₃)CH₂SCH₃ (S)-isomer | H | H | C—SC₃H₇-n | CH | CH |
| 14-405 | CH(CH₃)CH₂SCH₃ (S)-isomer | H | H | C—SCH₂CH═CH₂ | CH | CH |
| 14-406 | CH(CH₃)CH₂SCH₃ (S)-isomer | H | H | C—SCH₂CH(CH₃)₂ | CH | CH |
| 14-407 | CH(CH₃)CH₂SCH₃ (S)-isomer | H | H | C—SCF₃ | CH | CH |
| 14-408 | CH(CH₃)CH₂SCH₃ (S)-isomer | H | H | C—SCH₃ | CH | CH |
| 14-409 | CH(CH₃)CH₂SCH₃ (S)-isomer | H | H | C—SCH₂CF₃ | CH | CH |
| 14-410 | CH(CH₃)CH₂SCH₃ (S)-isomer | H | H | C—SCH₂Ph | CH | CH |
| 14-411 | CH(CH₃)CH₂SCH₃ (S)-isomer | H | H | C—SO₂CH₃ | CH | CH |
| 14-412 | CH(CH₃)CH₂SCH₃ (S)-isomer | H | H | C—SOCH₃ | CH | CH |
| 14-413 | CH(CH₃)CH₂SO₂CH₃ (S)-isomer | H | H | C—SO₂CH₃ | CH | CH |
| 14-414 | CH(CH₃)CH₂SCH₃ (S)-isomer | H | H | C—SO₂N(CH₃)₂ | CH | CH |
| 14-415 | CH(CH₃)CH₂SCH₃ (S)-isomer | H | H | C—SCH₃ | CH | CH |
| 14-416 | CH(CH₃)CH₂SCH₃ (S)-isomer | H | H | C—SC═C═CH₂ | CH | CH |
| 14-417 | CH(CH₃)CH₂SCH₃ (S)-isomer | H | H | C—SO₂C₂H₅ | CH | CH |
| 14-418 | CH(CH₃)CH₂SCH₃ (S)-isomer | H | H | C—SCH₃ | CH | CH |
| 14-419 | CH(CH₃)CH₂SCH₃ (S)-isomer | H | H | C—SCH₃ | CH | CH |
| 14-420 | CH(CH₃)CH₂SCH₃ (S)-isomer | H | H | C—SCH₃ | CH | CH |
| 14-421 | CH(CH₃)CH₂SCH₃ (S)-isomer | H | H | C—Cl | CH | CH |
| 14-422 | CH(CH₃)CH₂SOCH₃ (S)-isomer | H | H | C—Cl | CH | CH |
| 14-423 | CH(CH₃)CH₂SO₂CH₃ (S)-isomer | H | H | C—Cl | CH | CH |
| 14-424 | CH(CH₃)CH₂SCH₃ (S)-isomer | H | H | C—Cl | CH | CH |
| 14-425 | CH(CH₃)CH₂SOCH₃ (S)-isomer | H | H | C—Cl | CH | CH |
| 14-426 | CH(CH₃)CH₂SCH₃ (S)-isomer | H | H | C—SO₂CH₃ | CH | CH |
| 14-427 | CH(CH₃)CH₂SCH₃ (S)-isomer | H | H | C—SO₂CH₃ | CH | CH |
| 14-428 | C(CH₃)₂CON(C₂H₅)₂ | H | H | C—Cl | CH | CH |
| 14-429 | 4-methylthiophenyl | H | H | C—I | CH | CH |
| 14-430 | 4-methylsulfinylphenyl | H | H | C—I | CH | CH |
| 14-431 | 4-methylsulfonylphenyl | H | H | C—I | CH | CH |
| 14-432 | 3-methylthiophenyl | H | H | C—I | CH | CH |
| 14-433 | 6-(methylthio)pyridine-3-yl | H | H | C—I | CH | CH |
| 14-434 | 6-(methylsulfinyl)pyridine-3-yl | H | H | C—I | CH | CH |
| 14-435 | 6-(methylsulfonyl)pyridine-3-yl | H | H | C—I | CH | CH |
| 14-436 | C(CH₃)₂CH₂SCH₃ | H | H | N | CH | CH |
| 14-437 | C(CH₃)₂CH₂SCH₃ | H | H | CH | CH | CH |
| 14-438 | CH(CH₃)CH₂SCH₃ | H | H | CH | N | CH |
| 14-439 | CH(CH₃)CH₂SCH₃ (S)-isomer | H | H | C—NH₂ | CH | CH |
| 14-440 | CH(CH₃)CH₂SCH₃ (S)-isomer | H | H | C—S-cyclopentyl | CH | CH |
| 14-441 | CH(CH₃)CH₂SOCH₃ (S)-isomer | H | H | C—SO- | CH | CH |
| | | | | cyclopentyl | | |
| 14-442 | CH(CH₃)CH₂SO₂CH₃ (S)-isomer | H | H | C—SO₂- | CH | CH |
| | | | | cyclopentyl | | |
| 14-443 | CH(CH₃)CH₂SCH₃ (S)-isomer | H | H | C—NHC₂H₅ | CH | CH |
| 14-444 | CH(CH₃)CH₂SCH₃ (S)-isomer | H | H | C—N(CH₃)₂ | CH | CH |
| 14-445 | CH(CH₃)CH₂SCH₃ | H | H | C—I | CH | CH |
| 14-446 | CH(CH₃)CH₂SO₂CH₃ | H | H | C—I | CH | CH |
| 14-447 | CH(CH₃)CH₂SCH₃ | H | H | C—I | CH | CH |
| 14-448 | CH(CH₃)CH₂SCH₃ | H | H | C—I | CH | CH |
| 14-449 | CH(CH₃)CH₂SCH₃ | H | H | C—I | CH | CH |
| 14-450 | CH(CH₃)₂ | H | H | C—Cl | CH | CH |
| 14-451 | CH(CH₃)₂ | H | H | C—Cl | CH | CH |
| 14-452 | CH(CH₃)₂ | H | H | C—Cl | CH | CH |
| 14-453 | CH(CH₃)₂ | H | H | C—SCH₃ | CH | CH |
| 14-454 | CH(CH₃)₂ | H | H | C—SOCH₃ | CH | CH |
| 14-455 | CH(CH₃)₂ | H | H | C—SO₂CH₃ | CH | CH |
| 14-456 | CH(CH₃)CH₂SO₂CH₃ (S)-isomer | H | H | C-[2-(methoxycarbonyl)phenyl] | CH | CH |
| 14-457 | CH(CH₃)CH₂SO₂CH₃ (S)-isomer | H | H | C-(3,5-dichlorophenyl) | CH | CH |
| 14-458 | CH(CH₃)CH₂SO₂CH₃ (S)-isomer | H | H | C-(3,5-dimethyl | CH | CH |
| | | | | phenyl) | | |
| 14-459 | CH(CH₃)CH₂SO₂CH₃ (S)-isomer | H | H | C-(3-acetylphenyl) | CH | CH |
| 14-460 | CH(CH₃)CH₂SO₂CH₃ (S)-isomer | H | H | C-(3-ethoxy | CH | CH |
| | | | | phenyl) | | |
| 14-461 | CH(CH₃)CH₂SO₂CH₃ (S)-isomer | H | H | C-(3-methylthiophenyl) | CH | CH |
| 14-462 | CH(CH₃)CH₂SO₂CH₃ (S)-isomer | H | H | C-(4-formylphenyl) | CH | CH |
| 14-463 | CH(CH₃)CH₂SO₂CH₃ (S)-isomer | H | H | C-(4-cyanophenyl) | CH | CH |
| 14-464 | CH(CH₃)CH₂SO₂CH₃ (S)-isomer | H | H | C-(4-nitrophenyl) | CH | CH |
| 14-465 | CH(CH₃)CH₂SO₂CH₃ (S)-isomer | H | H | C-[4- | CH | CH |
| | | | | (trifluoromethoxy)phenyl] | | |
| 14-466 | CH(CH₃)CH₂SO₂CH₃ (S)-isomer | H | H | C-[4- | CH | CH |
| | | | | (trifluoromethylthio)phenyl] | | |
| 14-467 | CH(CH₃)CH₂SCH₃ (S)-isomer | H | H | C—Cl | CH | CH |
| 14-468 | CH(CH₃)CH₂SCH₃ (S)-isomer | H | H | C—Cl | CH | CH |
| 14-469 | CH(CH₃)CH₂SCH₃ (S)-isomer | H | H | C—Cl | CH | CH |
| 14-470 | CH(CH₃)CH₂SCH₃ (S)-isomer | H | H | C—Cl | CH | CH |
| 14-471 | CH(CH₃)CH₂SCH₃ (S)-isomer | H | H | C—Cl | CH | CH |
| 14-472 | CH(CH₃)CH₂SCH₃ (S)-isomer | H | H | C—Cl | CH | CH |
| 14-473 | CH(CH₃)CH₂SCH₃ (S)-isomer | H | H | C—Cl | CH | CH |
| 14-474 | CH(CH₃)CH₂SCH₃ (S)-isomer | H | H | C—Cl | CH | CH |
| 14-475 | CH(CH₃)CH₂SCH₃ (S)-isomer | H | H | C—Cl | CH | CH |
| 14-476 | CH(CH₃)CH₂SCH₃ (S)-isomer | H | H | C—Cl | CH | CH |
| 14-477 | CH(CH₃)CH₂SCH₃ (S)-isomer | H | H | C—Cl | CH | CH |
| 14-16 | CH(CH₃)CH₂SCH₃ (S)-isomer | H | H | C—C₂H₅ | CH | CH |
| 14-17 | CH(CH₃)CH₂SCH₃ (S)-isomer | H | H | C—OCH₃ | CH | CH |
| 14-18 | CH(CH₃)CH₂SCH₃ (S)-isomer | H | H | C—OCH₃ | CH | CH |
| 14-19 | CH₃ | H | H | C—Cl | CH | CH |
| 14-20 | CH(CH₃)CH₂SCH₃ (S)-isomer | H | H | C—Cl | CH | CH |
| 14-41 | CH(CH₃)CH₂SCH₃ (S)-isomer | H | H | C—Cl | CH | CH |

| No. | W⁴ | W⁵ | W⁶ | W⁷ | W⁸ | W⁹ | A | r | Q |
|---|---|---|---|---|---|---|---|---|---|
| 14-1 | CH | C—CH₃ | CH | C—(A)r—Q | CH | CH | CH₂ | 1 | Q34 |
| 14-2 | CH | C—CH₃ | CH | C—(A)r—Q | CH | CH | CH₂ | 1 | Q48 |
| 14-3 | CH | C—CH₃ | CH | C—(A)r—Q | CH | CH | CH₂ | 1 | Q48 |
| 14-4 | CH | C—CH₃ | CH | C—(A)r—Q | CH | CH | CH₂ | 1 | Q48 |
| 14-5 | CH | C—CH₃ | CH | C—(A)r—Q | CH | CH | CH₂ | 1 | Q48 |
| 14-6 | CH | C—CH₃ | CH | C—(A)r—Q | CH | CH | CH₂ | 1 | Q48 |
| 14-7 | CH | C—CH₃ | CH | C—(A)r—Q | CH | CH | CH₂ | 1 | Q34 |
| 14-8 | CH | C—CH₃ | CH | C—(A)r—Q | CH | CH | CH₂ | 1 | Q48 |
| 14-9 | CH | C—CH₃ | CH | C—(A)r—Q | CH | CH | CH₂ | 1 | Q48 |
| 14-10 | CH | C—CH₃ | CH | C—(A)r—Q | CH | CH | CH₂ | 1 | Q48 |
| 14-11 | CH | C—CH₃ | CH | C—(A)r—Q | CH | CH | CH₂ | 1 | Q48 |
| 14-12 | CH | C—CH₃ | CH | C—(A)r—Q | CH | CH | CH₂ | 1 | Q48 |
| 14-13 | CH | C—CH₃ | CH | C—(A)r—Q | CH | CH | CH₂ | 1 | Q34 |
| 14-14 | CH | C—CH₃ | CH | C—(A)r—Q | CH | CH | CH₂ | 1 | Q34 |
| 14-15 | CH | C—CH₃ | CH | C—(A)r—Q | CH | CH | CH₂ | 1 | Q34 |
| 14-21 | CH | C—CH₃ | CH | C—(A)r—Q | CH | CH | CH₂ | 1 | Q34 |
| 14-22 | CH | C—CH₃ | CH | C—(A)r—Q | CH | CH | CH₂ | 1 | Q34 |
| 14-23 | CH | C—CH₃ | CH | C—(A)r—Q | CH | CH | CH₂ | 1 | Q34 |
| 14-24 | CH | C—CH₃ | CH | C—(A)r—Q | CH | CH | — | 0 | Q34 |
| 14-25 | CH | C—CH₃ | CH | C—(A)r—Q | CH | CH | CH₂ | 1 | Q34 |
| 14-26 | CH | C—CH₃ | CH | C—(A)r—Q | CH | CH | CH₂ | 1 | Q34 |
| 14-27 | CH | C—CH₃ | CH | C—(A)r—Q | CH | CH | CH₂ | 1 | Q36 |
| 14-28 | CH | C—CH₃ | CH | C—(A)r—Q | CH | CH | CH₂ | 1 | Q34 |
| 14-29 | CH | C—CH₃ | CH | C—(A)r—Q | CH | CH | CH₂ | 1 | Q34 |
| 14-30 | CH | C—CH₃ | CH | C—(A)r—Q | CH | CH | CH₂ | 1 | Q34 |
| 14-31 | CH | C—CH₃ | N | C—(A)r—Q | CH | CH | CH₂ | 1 | Q34 |
| 14-32 | CH | C—CH₃ | CH | C—(A)r—Q | CH | CH | CH₂ | 1 | Q34 |
| 14-33 | CH | C—CH₃ | CH | C—(A)r—Q | CH | CH | CH₂ | 1 | Q34 |
| 14-34 | CH | C—CH₃ | CH | C—(A)r—Q | CH | CH | CH₂ | 1 | Q34 |
| 14-35 | CH | C—CH₃ | CH | C—(A)r—Q | CH | CH | CH₂ | 1 | Q34 |
| 14-36 | CH | C—CH₃ | CH | C—(A)r—Q | CH | CH | CH₂ | 1 | Q34 |
| 14-37 | CH | C—CH₃ | CH | C—(A)r—Q | CH | CH | CH₂ | 1 | Q34 |
| 14-38 | CH | C—CH₃ | CH | C—(A)r—Q | CH | CH | CH₂ | 1 | Q34 |
| 14-39 | CH | C—CH₃ | CH | C—(A)r—Q | CH | CH | CH₂ | 1 | Q34 |
| 14-40 | CH | C—CH₃ | CH | C—(A)r—Q | CH | CH | CH₂ | 1 | Q34 |
| 14-42 | CH | C—CH₃ | CH | C—(A)r—Q | CH | CH | CH₂ | 1 | Q34 |
| 14-43 | CH | C—CH₃ | CH | C—(A)r—Q | CH | CH | CH₂ | 1 | Q34 |
| 14-44 | CH | C—CH₃ | CH | C—(A)r—Q | CH | CH | CH₂ | 1 | Q34 |
| 14-45 | CH | C—CH₃ | CH | C—(A)r—Q | CH | CH | — | 0 | Q34 |
| 14-46 | CH | C—CH₃ | CH | C—(A)r—Q | CH | CH | CH₂ | 1 | Q34 |
| 14-47 | CH | C—CH₃ | CH | C—(A)r—Q | CH | CH | CH₂ | 1 | Q34 |
| 14-48 | CH | C—CH₃ | CH | C—(A)r—Q | CH | CH | CH₂ | 1 | Q34 |
| 14-49 | CH | C—CH₃ | CH | C—(A)r—Q | CH | CH | CH₂ | 1 | Q34 |
| 14-50 | CH | C—CH₃ | CH | C—(A)r—Q | CH | CH | CH₂ | 1 | Q34 |
| 14-51 | CH | C—CH₃ | CH | C—(A)r—Q | CH | CH | CH₂ | 1 | Q34 |
| 14-52 | CH | C—CH₃ | CH | C—(A)r—Q | CH | CH | CH₂ | 1 | Q34 |
| 14-53 | CH | C—CH₃ | CH | C—(A)r—Q | CH | CH | CH₂ | 1 | Q34 |
| 14-54 | CH | C—CH₃ | CH | C—(A)r—Q | CH | CH | — | 0 | Q34 |
| 14-55 | CH | C—CH₃ | CH | C—(A)r—Q | CH | CH | CH₂ | 1 | Q34 |
| 14-56 | CH | C—CH₃ | CH | C—(A)r—Q | CH | CH | CH₂ | 1 | Q34 |
| 14-57 | CH | C—CH₃ | CH | C—(A)r—Q | CH | CH | CH₂ | 1 | Q34 |
| 14-58 | CH | C—CH₃ | CH | C—(A)r—Q | CH | CH | — | 0 | Q34 |
| 14-59 | CH | C—CH₃ | CH | C—(A)r—Q | CH | CH | CH₂ | 1 | Q34 |
| 14-60 | CH | C—CH₃ | N | C—(A)r—Q | CH | CH | CH₂ | 1 | Q34 |
| 14-61 | CH | C—CH₃ | N | C—(A)r—Q | CH | CH | CH₂ | 1 | Q34 |
| 14-62 | CH | C—CH₃ | CH | C—(A)r—Q | CH | CH | CH₂ | 1 | Q34 |
| 14-63 | CH | C—CH₃ | CH | C—(A)r—Q | CH | CH | — | 0 | Q34 |
| 14-64 | CH | C—CH₃ | CH | C—(A)r—Q | CH | CH | CH₂ | 1 | Q34 |
| 14-65 | CH | C—CH₃ | CH | C—(A)r—Q | CH | CH | CH₂ | 1 | Q34 |
| 14-66 | CH | C—CH₃ | CH | C—(A)r—Q | CH | CH | CH₂ | 1 | Q34 |
| 14-67 | CH | C—CH₃ | CH | C—(A)r—Q | CH | CH | — | 0 | Q34 |
| 14-68 | CH | C—CH₃ | CH | C—(A)r—Q | CH | CH | CH₂ | 1 | Q34 |
| 14-69 | CH | C—CH₃ | CH | C—(A)r—Q | CH | CH | CH₂ | 1 | Q34 |
| 14-70 | CH | C—CH₃ | CH | C—(A)r—Q | CH | CH | CH₂ | 1 | Q34 |
| 14-71 | CH | C—CH₃ | CH | C—(A)r—Q | CH | CH | CH(CH₃) | 1 | Q34 |
| 14-72 | CH | C—CH₃ | CH | C—(A)r—Q | CH | CH | CH₂ | 1 | Q34 |
| 14-73 | CH | C—CH₃ | CH | C—(A)r—Q | CH | CH | CH₂ | 1 | Q34 |
| 14-74 | CH | C—CH₃ | CH | C—(A)r—Q | CH | CH | CH₂ | 1 | Q34 |
| 14-75 | CH | C—CH₃ | CH | C—(A)r—Q | CH | CH | CH₂ | 1 | Q34 |
| 14-76 | CH | C—CH₃ | CH | C—(A)r—Q | CH | CH | CH(CH₃) | 1 | Q34 |
| 14-77 | CH | C—CH₃ | CH | C—(A)r—Q | CH | CH | CH₂ | 1 | Q34 |
| 14-78 | CH | C—CH₃ | CH | C—(A)r—Q | CH | CH | CH₂ | 1 | Q34 |
| 14-79 | CH | C—CH₃ | CH | C—(A)r—Q | CH | CH | CH(CH₃) | 1 | Q34 |
| 14-80 | CH | C—CH₃ | CH | C—(A)r—Q | CH | CH | CH₂ | 1 | Q34 |
| 14-81 | CH | C—CH₃ | CH | C—(A)r—Q | CH | CH | CH₂ | 1 | Q34 |
| 14-82 | CH | C—CH₃ | CH | C—(A)r—Q | CH | CH | CH₂ | 1 | Q34 |
| 14-83 | CH | C—CH₃ | CH | C—(A)r—Q | CH | CH | CH₂ | 1 | Q34 |
| 14-84 | CH | C—CH₃ | CH | C—(A)r—Q | CH | CH | CH₂ | 1 | Q34 |
| 14-85 | CH | C—CH₃ | CH | C—(A)r—Q | CH | CH | CH₂ | 1 | Q34 |
| 14-86 | CH | C—CH₃ | CH | C—(A)r—Q | CH | CH | CH₂ | 1 | Q34 |
| 14-87 | CH | C—CH₃ | CH | C—(A)r—Q | CH | CH | CH₂ | 1 | Q34 |
| 14-88 | CH | C—CH₃ | CH | C—(A)r—Q | CH | CH | CH₂ | 1 | Q34 |
| 14-89 | CH | C—CH₃ | CH | C—(A)r—Q | CH | CH | CH₂ | 1 | Q34 |
| 14-90 | CH | C—CH₃ | CH | C—(A)r—Q | CH | CH | CH₂ | 1 | Q34 |
| 14-91 | CH | C—CH₃ | CH | C—(A)r—Q | CH | CH | CH₂ | 1 | Q34 |
| 14-92 | CH | C—CH₃ | CH | C—(A)r—Q | CH | CH | CH₂ | 1 | Q34 |
| 14-93 | CH | C—CH₃ | CH | C—(A)r—Q | CH | CH | CH₂ | 1 | Q34 |
| 14-94 | CH | C—CH₃ | CH | C—(A)r—Q | CH | CH | CH₂ | 1 | Q34 |
| 14-95 | CH | C—CH₃ | CH | C—(A)r—Q | CH | CH | CH₂ | 1 | Q34 |
| 14-96 | CH | C—CH₃ | CH | C—(A)r—Q | CH | CH | CH₂ | 1 | Q34 |
| 14-97 | CH | C—CH₃ | CH | C—(A)r—Q | CH | CH | CH₂ | 1 | Q34 |
| 14-98 | CH | C—CH₃ | CH | C—(A)r—Q | CH | CH | CH₂ | 1 | Q34 |
| 14-99 | CH | C—CH₃ | CH | C—(A)r—Q | CH | CH | CH₂ | 1 | Q34 |
| 14-100 | CH | C—CH₃ | CH | C—(A)r—Q | CH | CH | CH₂ | 1 | Q34 |
| 14-101 | CH | C—CH₃ | CH | C—(A)r—Q | CH | CH | CH₂ | 1 | Q34 |
| 14-102 | CH | C—CH₃ | CH | C—(A)r—Q | CH | CH | CH₂ | 1 | Q34 |
| 14-103 | CH | C—CH₃ | CH | C—(A)r—Q | CH | CH | CH₂ | 1 | Q34 |
| 14-104 | CH | C—CH₃ | CH | C—(A)r—Q | CH | CH | CH₂ | 1 | Q34 |
| 14-105 | CH | C—CH₃ | CH | C—(A)r—Q | CH | CH | CH₂ | 1 | Q34 |
| 14-106 | CH | C—CH₃ | CH | C—(A)r—Q | CH | CH | CH₂ | 1 | Q34 |
| 14-107 | CH | C—CH₃ | CH | C—(A)r—Q | CH | CH | CH₂ | 1 | Q34 |
| 14-108 | CH | C—CH₃ | CH | C—(A)r—Q | CH | CH | CH₂ | 1 | Q34 |
| 14-109 | CH | C—CH₃ | CH | C—(A)r—Q | CH | CH | CH₂ | 1 | Q34 |
| 14-110 | CH | C—CH₃ | CH | C—(A)r—Q | CH | CH | CH₂ | 1 | Q34 |
| 14-111 | CH | C—CH₃ | CH | C—(A)r—Q | CH | CH | CH₂ | 1 | Q34 |
| 14-112 | CH | C—CH₃ | CH | C—(A)r—Q | CH | CH | CH₂ | 1 | Q34 |
| 14-113 | CH | C—CH₃ | CH | C—(A)r—Q | CH | CH | CH₂ | 1 | Q34 |
| 14-114 | CH | C—CH₃ | CH | C—(A)r—Q | CH | CH | CH₂ | 1 | Q34 |
| 14-115 | CH | C—CH₃ | CH | C—(A)r—Q | CH | CH | CH₂ | 1 | Q34 |
| 14-116 | CH | C—CH₃ | CH | C—(A)r—Q | CH | CH | CH₂ | 1 | Q34 |
| 14-117 | CH | C—CH₃ | CH | C—(A)r—Q | CH | CH | CH₂ | 1 | Q34 |
| 14-118 | CH | C—CH₃ | CH | C—(A)r—Q | CH | CH | CH₂ | 1 | Q34 |
| 14-119 | CH | C—CH₃ | CH | C—(A)r—Q | CH | CH | CH₂ | 1 | Q34 |
| 14-120 | CH | C—CH₃ | CH | C—(A)r—Q | CH | CH | CH₂ | 1 | Q34 |
| 14-121 | CH | C—CH₃ | CH | C—(A)r—Q | CH | CH | CH₂ | 1 | Q34 |
| 14-122 | CH | C—CH₃ | CH | C—(A)r—Q | CH | CH | CH₂ | 1 | Q34 |
| 14-123 | CH | C—CH₃ | CH | C—(A)r—Q | CH | CH | CH₂ | 1 | Q34 |
| 14-124 | CH | C—CH₃ | CH | C—(A)r—Q | CH | CH | CH₂ | 1 | Q34 |
| 14-125 | CH | C—CH₃ | CH | C—(A)r—Q | CH | CH | CH₂ | 1 | Q34 |
| 14-126 | CH | C—CH₃ | CH | C—(A)r—Q | CH | CH | CH₂ | 1 | Q34 |
| 14-127 | CH | C—CH₃ | CH | C—(A)r—Q | CH | CH | CH₂ | 1 | Q34 |
| 14-128 | CH | C—CH₃ | CH | C—(A)r—Q | CH | CH | CH₂ | 1 | Q34 |
| 14-129 | CH | C—CH₃ | CH | C—(A)r—Q | CH | CH | CH₂ | 1 | Q34 |
| 14-130 | CH | C—CH₃ | CH | C—(A)r—Q | CH | CH | CH₂ | 1 | Q34 |
| 14-131 | CH | C—CH₃ | CH | C—(A)r—Q | CH | CH | CH₂ | 1 | Q34 |
| 14-132 | CH | C—CH₃ | CH | C—(A)r—Q | CH | CH | CH₂ | 1 | Q34 |
| 14-133 | CH | C—CH₃ | CH | C—(A)r—Q | CH | CH | CH₂ | 1 | Q34 |
| 14-134 | CH | C—CH₃ | CH | C—(A)r—Q | CH | CH | CH₂ | 1 | Q34 |
| 14-135 | CH | C—CH₃ | CH | C—(A)r—Q | CH | CH | CH₂ | 1 | Q34 |
| 14-136 | CH | C—CH₃ | CH | C—(A)r—Q | CH | CH | CH₂ | 1 | Q34 |
| 14-137 | CH | C—CH₃ | CH | C—(A)r—Q | CH | CH | CH₂ | 1 | Q34 |
| 14-138 | CH | C—CH₃ | CH | C—(A)r—Q | CH | CH | CH₂ | 1 | Q34 |
| 14-139 | CH | C—CH₃ | CH | C—(A)r—Q | CH | CH | CH₂ | 1 | Q34 |
| 14-140 | CH | C—CH₃ | CH | C—(A)r—Q | CH | CH | CH₂ | 1 | Q34 |
| 14-141 | CH | C—CH₃ | CH | C—(A)r—Q | CH | CH | CH₂ | 1 | Q34 |
| 14-142 | CH | C—CH₃ | CH | C—(A)r—Q | CH | CH | CH₂ | 1 | Q34 |
| 14-143 | CH | C—CH₃ | CH | C—(A)r—Q | CH | CH | CH₂ | 1 | Q34 |
| 14-144 | CH | C—CH₃ | CH | C—(A)r—Q | CH | CH | CH₂ | 1 | Q34 |
| 14-145 | CH | C—CH₃ | CH | C—(A)r—Q | CH | CH | — | 0 | Q38 |
| 14-146 | CH | C—CH₃ | CH | C—(A)r—Q | CH | CH | CH₂ | 1 | Q38 |
| 14-147 | CH | C—CH₃ | CH | C—(A)r—Q | CH | CH | — | 0 | Q38 |
| 14-148 | CH | C—CH₃ | CH | C—(A)r—Q | CH | CH | CH₂ | 1 | Q36 |
| 14-149 | CH | C—CH₃ | CH | C—(A)r—Q | CH | CH | CH₂ | 1 | Q35 |
| 14-150 | CH | C—CH₃ | CH | C—(A)r—Q | CH | CH | CH₂ | 1 | Q36 |
| 14-151 | CH | C—CH₃ | CH | C—(A)r—Q | CH | CH | CH₂ | 1 | Q35 |
| 14-152 | CH | C—CH₃ | CH | C—(A)r—Q | CH | CH | CH₂ | 1 | Q35 |
| 14-153 | CH | C—CH₃ | CH | C—(A)r—Q | CH | CH | CH₂ | 1 | Q36 |
| 14-154 | CH | C—CH₃ | CH | C—(A)r—Q | CH | CH | CH₂ | 1 | Q35 |
| 14-155 | CH | C—CH₃ | CH | C—(A)r—Q | CH | CH | CH₂ | 1 | Q35 |
| 14-156 | CH | C—CH₃ | CH | C—(A)r—Q | CH | CH | CH₂ | 1 | Q36 |
| 14-157 | CH | C—CH₃ | CH | C—(A)r—Q | CH | CH | CH₂ | 1 | Q41 |
| 14-158 | CH | C—CH₃ | CH | C—(A)r—Q | CH | CH | CH₂ | 1 | Q41 |
| 14-159 | CH | C—CH₃ | CH | C—(A)r—Q | CH | CH | CH₂ | 1 | Q41 |
| 14-160 | CH | C—CH₃ | CH | C—(A)r—Q | CH | CH | CH₂ | 1 | Q41 |
| 14-161 | CH | C—CH₃ | CH | C—(A)r—Q | CH | CH | CH₂ | 1 | Q41 |
| 14-162 | CH | C—CH₃ | CH | C—(A)r—Q | CH | CH | CH₂ | 1 | Q41 |
| 14-163 | CH | C—CH₃ | CH | C—(A)r—Q | CH | CH | CH₂ | 1 | Q48 |
| 14-164 | CH | C—Cl | CH | C—(A)r—Q | CH | CH | CH₂ | 1 | Q34 |
| 14-165 | CH | C—Cl | CH | C—(A)r—Q | CH | CH | CH₂ | 1 | Q34 |
| 14-166 | CH | C—Cl | CH | C—(A)r—Q | CH | CH | CH₂ | 1 | Q34 |
| 14-167 | CH | C—Cl | CH | C—(A)r—Q | CH | CH | CH₂ | 1 | Q34 |
| 14-168 | CH | C—Cl | CH | C—(A)r—Q | CH | CH | CH₂ | 1 | Q34 |
| 14-169 | CH | C—CH₃ | CH | C—(A)r—Q | CH | CH | CH₂ | 1 | Q34 |
| 14-170 | CH | C—CH₃ | CH | C—(A)r—Q | CH | CH | CH₂ | 1 | Q34 |
| 14-171 | CH | C—CH₃ | CH | C—(A)r—Q | CH | CH | CH₂ | 1 | Q34 |
| 14-172 | CH | C—CH₃ | CH | C—(A)r—Q | CH | CH | CH₂ | 1 | Q34 |
| 14-173 | CH | C—CH₃ | CH | C—(A)r—Q | CH | CH | CH₂ | 1 | Q34 |
| 14-174 | CH | C—CH₃ | CH | C—(A)r—Q | CH | CH | CH₂ | 1 | Q34 |
| 14-175 | CH | C—CH₃ | CH | C—(A)r—Q | CH | CH | CH₂ | 1 | Q34 |
| 14-176 | CH | C—CH₃ | CH | C—(A)r—Q | CH | CH | CH₂ | 1 | Q34 |
| 14-177 | CH | C—CH₃ | CH | C—(A)r—Q | CH | CH | CH₂ | 1 | Q34 |
| 14-178 | CH | C—CH₃ | CH | C—(A)r—Q | CH | CH | CH₂ | 1 | Q34 |
| 14-179 | CH | C—CH₃ | CH | C—(A)r—Q | CH | CH | CH₂ | 1 | Q34 |
| 14-180 | CH | C—CH₃ | CH | C—(A)r—Q | CH | CH | CH₂ | 1 | Q34 |
| 14-181 | CH | C—CH₃ | CH | C—(A)r—Q | CH | CH | CH₂ | 1 | Q34 |
| 14-182 | CH | C—CH₃ | CH | C—(A)r—Q | CH | CH | CH₂ | 1 | Q34 |
| 14-183 | CH | C—CH₃ | CH | C—(A)r—Q | CH | CH | CH₂ | 1 | Q34 |
| 14-184 | CH | C—CH₃ | CH | C—(A)r—Q | CH | CH | CH₂ | 1 | Q34 |
| 14-185 | CH | C—CH₃ | CH | C—(A)r—Q | CH | CH | CH₂ | 1 | Q34 |
| 14-186 | CH | C—CH₃ | CH | C—(A)r—Q | CH | CH | CH₂ | 1 | Q34 |
| 14-187 | CH | C—CH₃ | CH | C—(A)r—Q | CH | CH | CH₂ | 1 | Q34 |
| 14-188 | CH | C—CH₃ | CH | C—(A)r—Q | CH | CH | CH₂ | 1 | Q34 |
| 14-189 | CH | C—CH₃ | CH | C—(A)r—Q | CH | CH | CH₂ | 1 | Q34 |
| 14-190 | CH | C—CH₃ | CH | C—(A)r—Q | CH | CH | CH₂ | 1 | Q34 |
| 14-191 | CH | C—CH₃ | CH | C—(A)r—Q | CH | CH | CH₂ | 1 | Q34 |
| 14-192 | CH | C—CH₃ | CH | C—(A)r—Q | CH | CH | CH₂ | 1 | Q34 |
| 14-193 | CH | C—CH₃ | CH | C—(A)r—Q | CH | CH | CH₂ | 1 | Q34 |
| 14-194 | CH | CH | CH | C—(A)r—Q | CH | CH | CH₂ | 1 | Q34 |
| 14-195 | CH | CH | CH | C—(A)r—Q | CH | CH | CH₂ | 1 | Q34 |
| 14-196 | CH | CH | CH | C—(A)r—Q | CH | CH | CH₂ | 1 | Q34 |
| 14-197 | CH | C—Br | CH | C—(A)r—Q | CH | CH | CH₂ | 1 | Q34 |
| 14-198 | CH | C—CH₃ | CH | C—(A)r—Q | CH | CH | CH₂ | 1 | Q34 |
| 14-199 | CH | C—CH₃ | CH | C—(A)r—Q | CH | CH | CH₂ | 1 | Q34 |
| 14-200 | CH | C—CH₃ | CH | C—(A)r—Q | CH | CH | CH₂ | 1 | Q34 |
| 14-201 | CH | C—CH₃ | CH | C—(A)r—Q | CH | CH | CH₂ | 1 | Q34 |
| 14-202 | CH | C—CH₃ | CH | C—(A)r—Q | CH | CH | CH₂ | 1 | Q34 |
| 14-203 | CH | C—CH₃ | CH | C—(A)r—Q | CH | CH | CH₂ | 1 | Q34 |
| 14-204 | CH | C—CH₃ | CH | C—(A)r—Q | CH | CH | CH₂ | 1 | Q34 |
| 14-205 | CH | C—CH₃ | CH | C—(A)r—Q | CH | CH | CH₂ | 1 | Q34 |
| 14-206 | CH | C—CH₃ | CH | C—(A)r—Q | CH | CH | CH₂ | 1 | Q34 |
| 14-207 | CH | C—CH₃ | CH | C—(A)r—Q | CH | CH | CH₂ | 1 | Q34 |
| 14-208 | CH | C—CH₃ | CH | C—(A)r—Q | CH | CH | CH₂ | 1 | Q34 |
| 14-209 | CH | C—CH₃ | CH | C—(A)r—Q | CH | CH | CH₂ | 1 | Q34 |
| 14-210 | CH | C—CH₃ | CH | C—(A)r—Q | CH | CH | CH₂ | 1 | Q34 |
| 14-211 | CH | C—CH₃ | CH | C—(A)r—Q | CH | CH | CH₂ | 1 | Q34 |
| 14-212 | CH | C—CH₃ | CH | C—(A)r—Q | CH | CH | CH₂ | 1 | Q34 |
| 14-213 | CH | C—CH₃ | CH | C—(A)r—Q | CH | CH | CH₂ | 1 | Q34 |
| 14-214 | CH | C—CH₃ | CH | C—(A)r—Q | CH | CH | CH₂ | 1 | Q34 |
| 14-215 | CH | C—CH₃ | CH | C—(A)r—Q | CH | CH | CH₂ | 1 | Q34 |
| 14-216 | CH | C—CH₃ | CH | C—(A)r—Q | CH | CH | CH₂ | 1 | Q34 |
| 14-217 | CH | C—CH₃ | CH | C—(A)r—Q | CH | CH | CH₂ | 1 | Q34 |
| 14-218 | CH | C—CH₃ | CH | C—(A)r—Q | CH | CH | CH₂ | 1 | Q34 |
| 14-219 | CH | C—CH₃ | CH | C—(A)r—Q | CH | CH | CH₂ | 1 | Q34 |
| 14-220 | CH | C—CH₃ | CH | C—(A)r—Q | CH | CH | CH₂ | 1 | Q34 |
| 14-221 | CH | C—CH₃ | CH | C—(A)r—Q | CH | CH | CH₂ | 1 | Q34 |
| 14-222 | CH | C—CH₃ | CH | C—(A)r—Q | CH | CH | CH₂ | 1 | Q34 |
| 14-223 | CH | C—CH₃ | CH | C—(A)r—Q | CH | CH | CH₂ | 1 | Q34 |
| 14-224 | CH | C—CH₃ | CH | C—(A)r—Q | CH | CH | CH₂ | 1 | Q34 |
| 14-225 | CH | C—CH₃ | CH | C—(A)r—Q | CH | CH | CH₂ | 1 | Q34 |
| 14-226 | CH | C—CH₃ | CH | C—(A)r—Q | CH | CH | CH₂ | 1 | Q34 |
| 14-227 | CH | C—CH₃ | CH | C—(A)r—Q | CH | CH | CH₂ | 1 | Q34 |
| 14-228 | CH | C—CH₃ | CH | C—(A)r—Q | CH | CH | CH₂ | 1 | Q40 |
| 14-229 | CH | C—CH₃ | CH | C—(A)r—Q | CH | CH | CH₂ | 1 | Q39 |
| 14-230 | CH | C—CH₃ | CH | C—(A)r—Q | CH | CH | CH₂ | 1 | Q39 |
| 14-231 | CH | C—CH₃ | CH | C—(A)r—Q | CH | CH | CH₂ | 1 | Q39 |
| 14-232 | CH | C—CH₃ | CH | C—(A)r—Q | CH | CH | — | 0 | Q38 |
| 14-233 | CH | C—CH₃ | CH | C—(A)r—Q | CH | CH | — | 0 | Q38 |
| 14-234 | CH | C—CH₃ | CH | C—(A)r—Q | CH | CH | — | 0 | Q38 |
| 14-235 | CH | C—CH₃ | CH | C—(A)r—Q | CH | CH | — | 0 | Q38 |
| 14-236 | CH | C—CH₃ | CH | C—(A)r—Q | CH | CH | CH₂ | 1 | Q43 |
| 14-237 | CH | C—CH₃ | CH | C—(A)r—Q | CH | CH | CH₂ | 1 | Q43 |
| 14-238 | CH | C—CH₃ | CH | C—(A)r—Q | CH | CH | CH₂ | 1 | Q41 |
| 14-239 | CH | C—CH₃ | CH | C—(A)r—Q | CH | CH | CH₂ | 1 | Q47 |
| 14-240 | CH | C—CH₃ | CH | C—(A)r—Q | CH | CH | CH₂ | 1 | Q47 |
| 14-241 | CH | C—CH₃ | CH | C—(A)r—Q | CH | CH | CH₂ | 1 | Q47 |
| 14-242 | CH | C—CH₃ | CH | C—(A)r—Q | CH | CH | CH₂ | 1 | Q48 |
| 14-243 | CH | C—CH₃ | CH | C—(A)r—Q | CH | CH | CH₂ | 1 | Q48 |
| 14-244 | CH | C—CH₃ | CH | C—(A)r—Q | CH | CH | CH₂ | 1 | Q48 |
| 14-245 | CH | C—CH₃ | CH | C—(A)r—Q | CH | CH | CH₂ | 1 | Q48 |
| 14-246 | CH | C—CH₃ | CH | C—(A)r—Q | CH | CH | OCH₂CH₂ | 1 | Q48 |
| 14-247 | CH | C—CH₃ | CH | C—(A)r—Q | CH | CH | OCH₂CH₂ | 1 | Q48 |
| 14-249 | CH | C—CH₃ | CH | C—(A)r—Q | CH | CH | CH₂ | 1 | Q48 |
| 14-250 | CH | C—CH₃ | CH | C—(A)r—Q | CH | CH | CH₂ | 1 | Q48 |
| 14-251 | CH | C—CH₃ | CH | C—(A)r—Q | CH | CH | CH₂ | 1 | Q48 |
| 14-252 | CH | C—CH₃ | CH | C—(A)r—Q | CH | CH | CH₂ | 1 | Q48 |
| 14-253 | CH | C—CH₃ | CH | C—(A)r—Q | CH | CH | CH₂ | 1 | Q48 |
| 14-254 | CH | C—CH₃ | CH | C—(A)r—Q | CH | CH | CH₂ | 1 | Q48 |
| 14-255 | CH | C—CH₃ | CH | C—(A)r—Q | CH | CH | CH₂ | 1 | Q48 |
| 14-256 | CH | C—CH₃ | CH | C—(A)r—Q | CH | CH | CH₂ | 1 | Q48 |
| 14-257 | CH | C—CH₃ | CH | C—(A)r—Q | CH | CH | CH₂ | 1 | Q48 |
| 14-258 | CH | C—CH₃ | CH | C—(A)r—Q | CH | CH | CH₂ | 1 | Q48 |
| 14-259 | CH | C—CH₃ | CH | C—(A)r—Q | CH | CH | CH₂ | 1 | Q48 |
| 14-260 | CH | C—CH₃ | CH | C—(A)r—Q | CH | CH | CH₂ | 1 | Q48 |
| 14-261 | CH | C—CH₃ | CH | C—(A)r—Q | CH | CH | CH₂ | 1 | Q48 |
| 14-262 | CH | C—CH₃ | CH | C—(A)r—Q | CH | CH | CH₂ | 1 | Q34 |
| 14-263 | CH | C—CH₃ | CH | C—(A)r—Q | CH | CH | CH₂ | 1 | Q34 |
| 14-264 | CH | C—CH₃ | CH | C—(A)r—Q | CH | CH | CH₂ | 1 | Q34 |
| 14-265 | CH | C—CH₃ | CH | C—(A)r—Q | CH | CH | CH₂ | 1 | Q34 |
| 14-266 | CH | C—CH₃ | CH | C—(A)r—Q | CH | CH | CH₂ | 1 | Q34 |
| 14-267 | CH | C—CH₃ | CH | C—(A)r—Q | CH | CH | CH₂ | 1 | Q34 |
| 14-268 | CH | C—CH₃ | CH | C—(A)r—Q | N | CH | CH₂ | 1 | Q34 |
| 14-269 | CH | C—CH₃ | CH | C—(A)r—Q | N | CH | CH₂ | 1 | Q34 |
| 14-270 | CH | C—CH₃ | CH | C—(A)r—Q | N | CH | CH₂ | 1 | Q34 |
| 14-271 | CH | C—CH₃ | CH | C—(A)r—Q | N | CH | CH₂ | 1 | Q34 |
| 14-272 | CH | C—CH₃ | CH | C—(A)r—Q | N | CH | CH₂ | 1 | Q34 |
| 14-273 | CH | C—CH₃ | CH | C—(A)r—Q | N | CH | CH₂ | 1 | Q34 |
| 14-274 | CH | C—CH₃ | CH | C—(A)r—Q | N | CH | CH₂ | 1 | Q34 |
| 14-275 | CH | C—CH₃ | CH | C—(A)r—Q | N | CH | CH₂ | 1 | Q34 |
| 14-276 | CH | C—CH₃ | CH | C—(A)r—Q | N | CH | CH₂ | 1 | Q34 |
| 14-277 | CH | C—CH₃ | CH | C—(A)r—Q | N | CH | CH₂ | 1 | Q34 |
| 14-278 | CH | C—CH₃ | CH | C—(A)r—Q | N | CH | CH₂ | 1 | Q34 |
| 14-279 | CH | C—CH₃ | CH | C—(A)r—Q | N | CH | CH₂ | 1 | Q34 |
| 14-280 | CH | C—CH₃ | CH | C—(A)r—Q | N | CH | CH₂ | 1 | Q34 |
| 14-281 | CH | C—CH₃ | CH | C—(A)r—Q | N | CH | CH₂ | 1 | Q34 |
| 14-282 | CH | C—CH₃ | CH | C—(A)r—Q | N | CH | CH₂ | 1 | Q34 |
| 14-283 | CH | C—CH₃ | CH | C—(A)r—Q | N | CH | CH₂ | 1 | Q34 |
| 14-284 | CH | C—CH₃ | CH | C—(A)r—Q | N | CH | CH₂ | 1 | Q34 |
| 14-285 | CH | C—CH₃ | CH | C—(A)r—Q | N | CH | CH₂ | 1 | Q34 |
| 14-286 | CH | C—CH₃ | N | C—(A)r—Q | CH | CH | CH₂ | 1 | Q34 |
| 14-287 | CH | C—CH₃ | N | C—(A)r—Q | CH | CH | CH₂ | 1 | Q34 |
| 14-288 | CH | C—CH₃ | N | C—(A)r—Q | CH | CH | CH₂ | 1 | Q34 |
| 14-289 | CH | C—CH₃ | N | C—(A)r—Q | CH | CH | CH₂ | 1 | Q34 |
| 14-290 | CH | C—CH₃ | N | C—(A)r—Q | CH | CH | CH₂ | 1 | Q34 |
| 14-291 | CH | C—CH₃ | N | C—(A)r—Q | CH | CH | CH₂ | 1 | Q34 |
| 14-292 | CH | C—CH₃ | CH | C—(A)r—Q | CH | CH | CH₂ | 1 | Q34 |
| 14-293 | CH | C—CH₃ | CH | C—(A)r—Q | CH | CH | CH₂ | 1 | Q34 |
| 14-294 | CH | C—CH₃ | CH | C—(A)r—Q | CH | CH | CH₂ | 1 | Q34 |
| 14-295 | CH | C—CH₃ | CH | C—(A)r—Q | CH | CH | CH₂ | 1 | Q34 |
| 14-296 | CH | C—CH₃ | CH | C—(A)r—Q | CH | CH | CH₂ | 1 | Q34 |
| 14-297 | CH | C—CH₃ | CH | C—(A)r—Q | CH | CH | CH₂ | 1 | Q34 |
| 14-298 | CH | C—CH₃ | CH | C—(A)r—Q | CH | CH | CH₂ | 1 | Q34 |
| 14-299 | CH | C—CH₃ | CH | C—(A)r—Q | CH | CH | CH₂ | 1 | Q47 |
| 14-300 | CH | C—CH₃ | CH | C—(A)r—Q | CH | CH | CH₂ | 1 | Q34 |
| 14-301 | CH | C—CH₃ | CH | C—(A)r—Q | CH | CH | CH₂ | 1 | Q34 |
| 14-302 | CH | C—CH₃ | N | C—(A)r—Q | CH | CH | CH₂ | 1 | Q34 |
| 14-303 | CH | C—CH₃ | CH | C—(A)r—Q | CH | CH | CH₂ | 1 | Q34 |
| 14-304 | CH | C—CH₃ | CH | C—(A)r—Q | CH | CH | CH₂ | 1 | Q36 |
| 14-305 | CH | C—CH₃ | CH | C—(A)r—Q | CH | CH | CH₂ | 1 | Q47 |
| 14-306 | CH | C—CH₃ | N | C—(A)r—Q | CH | CH | CH₂ | 1 | Q47 |
| 14-307 | CH | C—CH₃ | N | C—(A)r—Q | CH | CH | CH₂ | 1 | Q47 |
| 14-308 | CH | C—CH₃ | CH | C—(A)r—Q | CH | CH | CH₂ | 1 | Q34 |
| 14-309 | CH | C—CH₃ | N | C—(A)r—Q | CH | CH | CH₂ | 1 | Q34 |
| 14-310 | CH | C—CH₃ | N | C—(A)r—Q | CH | CH | CH₂ | 1 | Q34 |
| 14-311 | CH | C—CH₃ | N | C—(A)r—Q | CH | CH | CH₂ | 1 | Q34 |
| 14-312 | CH | C—CH₃ | CH | C—(A)r—Q | CH | CH | CH₂ | 1 | Q34 |
| 14-313 | CH | C—CH₃ | CH | C—(A)r—Q | CH | CH | CH₂ | 1 | Q34 |
| 14-314 | CH | C—CH₃ | CH | C—(A)r—Q | CH | CH | C(CF₃)₂ | 1 | Q34 |
| 14-315 | CH | C—CH₃ | CH | C—(A)r—Q | CH | CH | CH(CF₃) | 1 | Q34 |
| 14-316 | CH | C—CH₃ | CH | C—(A)r—Q | CH | CH | CH₂ | 1 | Q34 |
| 14-317 | CH | C—CH₃ | CH | C—(A)r—Q | CH | CH | CH₂ | 1 | Q39 |
| 14-318 | CH | C—CH₃ | CH | C—(A)r—Q | CH | CH | CH₂ | 1 | Q39 |
| 14-319 | CH | C—CH₃ | CH | C—(A)r—Q | CH | CH | CH₂ | 1 | Q39 |
| 14-320 | CH | C—CH₃ | N | C—(A)r—Q | CH | CH | CH₂ | 1 | Q39 |
| 14-321 | CH | C—CH₃ | N | C—(A)r—Q | CH | CH | CH₂ | 1 | Q39 |
| 14-322 | CH | C—CH₃ | N | C—(A)r—Q | CH | CH | CH₂ | 1 | Q39 |
| 14-323 | CH | C—CH₃ | N | C—(A)r—Q | CH | CH | CH₂ | 1 | Q39 |
| 14-324 | CH | C—CH₃ | CH | C—(A)r—Q | CH | CH | CH₂ | 1 | Q39 |
| 14-325 | CH | C—CH₃ | CH | C—(A)r—Q | CH | CH | CH₂ | 1 | Q39 |
| 14-326 | CH | C—CH₃ | CH | C—(A)r—Q | CH | CH | CH₂ | 1 | Q39 |
| 14-327 | CH | C—CH₃ | CH | C—(A)r—Q | CH | CH | CH₂ | 1 | Q39 |
| 14-328 | CH | C—CH₃ | CH | C—(A)r—Q | CH | CH | CH₂ | 1 | Q39 |
| 14-329 | CH | C—CH₃ | CH | C—(A)r—Q | CH | CH | CH₂ | 1 | Q39 |
| 14-330 | CH | C—CH₃ | CH | C—(A)r—Q | CH | CH | CH₂ | 1 | Q39 |
| 14-331 | CH | C—CH₃ | CH | C—(A)r—Q | CH | CH | CH₂ | 1 | Q39 |
| 14-332 | CH | C—CH₃ | CH | C—(A)r—Q | CH | CH | CH₂ | 1 | Q39 |
| 14-333 | CH | C—CH₃ | CH | C—(A)r—Q | CH | CH | CH₂ | 1 | Q39 |
| 14-334 | CH | C—CH₃ | CH | C—(A)r—Q | CH | CH | CH₂ | 1 | Q39 |
| 14-335 | CH | C—CH₃ | CH | C—(A)r—Q | CH | CH | CH₂ | 1 | Q39 |
| 14-336 | CH | C—CH₃ | CH | C—(A)r—Q | CH | CH | CH₂ | 1 | Q39 |
| 14-337 | CH | C—F | CH | C—(A)r—Q | CH | CH | CH₂ | 1 | Q34 |
| 14-338 | CH | CH | C—F | C—(A)r—Q | CH | CH | CH₂ | 1 | Q34 |
| 14-339 | CH | CH | C—F | C—(A)r—Q | CH | CH | CH₂ | 1 | Q34 |
| 14-340 | CH | CH | C—F | C—(A)r—Q | CH | CH | CH₂ | 1 | Q34 |
| 14-341 | C—SC≡CCH₃ | C—CH₃ | CH | C—(A)r—Q | CH | CH | CH₂ | 1 | Q34 |
| 14-342 | C—CH═CH₂ | C—CH₃ | CH | C—(A)r—Q | CH | CH | CH₂ | 1 | Q34 |
| 14-343 | CH | C—CH₃ | CH | C—(A)r—Q | CH | CH | CH₂ | 1 | Q34 |
| 14-344 | CH | C—CH₃ | CH | C—(A)r—Q | CH | CH | CH₂ | 1 | Q34 |
| 14-345 | CH | C—CH₃ | CH | C—(A)r—Q | CH | CH | CH₂ | 1 | Q34 |
| 14-346 | CH | C—CH₃ | CH | C—(A)r—Q | CH | CH | CH₂ | 1 | Q34 |
| 14-347 | CH | C—CH₃ | CH | C—(A)r—Q | CH | CH | CH₂ | 1 | Q34 |
| 14-348 | CH | C—CH₃ | CH | C—(A)r—Q | CH | CH | CH₂ | 1 | Q47 |
| 14-349 | CH | C—CH₃ | CH | C—(A)r—Q | CH | CH | CH₂ | 1 | Q47 |
| 14-350 | CH | C—CH₃ | N | C—(A)r—Q | CH | CH | CH₂ | 1 | Q34 |
| 14-351 | CH | C—CH₃ | CH | C—(A)r—Q | CH | CH | CH₂ | 1 | Q34 |
| 14-352 | CH | C—CH₃ | CH | C—(A)r—Q | CH | CH | CH₂ | 1 | Q34 |
| 14-353 | CH | C—CH₃ | CH | C—(A)r—Q | CH | CH | CH₂ | 1 | Q34 |
| 14-354 | CH | CH | CH | C—(A)r—Q | CH | CH | CH₂ | 1 | Q34 |
| 14-355 | CH | CH | CH | C—(A)r—Q | CH | CH | CH₂ | 1 | Q34 |
| 14-356 | CH | C—CH₃ | CH | C—(A)r—Q | CH | CH | CH₂ | 1 | Q34 |
| 14-357 | CH | C—CH₃ | CH | C—(A)r—Q | CH | CH | CH₂ | 1 | Q34 |
| 14-358 | CH | C—CH₃ | CH | C—(A)r—Q | CH | CH | CH₂ | 1 | Q34 |
| 14-359 | CH | C—CH₃ | CH | C—(A)r—Q | CH | CH | CH₂ | 1 | Q34 |
| 14-360 | CH | C—CH₃ | CH | C—(A)r—Q | CH | CH | CH₂ | 1 | Q34 |
| 14-361 | CH | C—CH₃ | CH | C—(A)r—Q | CH | CH | CH₂ | 1 | Q34 |
| 14-362 | CH | C—CH₃ | CH | C—(A)r—Q | CH | CH | CH₂ | 1 | Q34 |
| 14-363 | CH | C—CH₃ | CH | C—(A)r—Q | CH | CH | CH₂ | 1 | Q34 |
| 14-364 | CH | C—CH₃ | N | C—(A)r—Q | CH | CH | CH₂ | 1 | Q34 |
| 14-365 | CH | C—CH₃ | N | C—(A)r—Q | CH | CH | CH₂ | 1 | Q34 |
| 14-366 | CH | C—CH₃ | N | C—(A)r—Q | CH | CH | CH₂ | 1 | Q34 |
| 14-367 | CH | C—CH₃ | N | C—(A)r—Q | CH | CH | CH₂ | 1 | Q34 |
| 14-368 | CH | C—CH₃ | N | C—(A)r—Q | CH | CH | CH₂ | 1 | Q34 |
| 14-369 | CH | C—CH₃ | N | C—(A)r—Q | CH | CH | CH₂ | 1 | Q34 |
| 14-370 | CH | C—CH₃ | CH | C—(A)r—Q | CH | CH | CH₂ | 1 | Q34 |
| 14-371 | CH | C—CH₃ | CH | C—(A)r—Q | CH | CH | CH₂ | 1 | Q34 |
| 14-372 | CH | C—CH₃ | CH | C—(A)r—Q | CH | CH | CH₂ | 1 | Q34 |
| 14-373 | CH | C—CH₃ | CH | C—(A)r—Q | CH | CH | CH₂ | 1 | Q34 |
| 14-374 | CH | C—CH₃ | CH | C—(A)r—Q | CH | CH | CH₂ | 1 | Q34 |
| 14-375 | CH | C—CH₃ | CH | C—(A)r—Q | CH | CH | CH₂ | 1 | Q34 |
| 14-376 | CH | C—CH₃ | CH | C—(A)r—Q | CH | CH | CH₂ | 1 | Q34 |
| 14-377 | CH | C—CH₃ | CH | C—(A)r—Q | CH | CH | CH₂ | 1 | Q34 |
| 14-378 | CH | C—CH₃ | CH | C—(A)r—Q | CH | CH | CH₂ | 1 | Q34 |
| 14-379 | CH | C—CH₃ | CH | C—(A)r—Q | CH | CH | CH₂ | 1 | Q34 |
| 14-380 | CH | C—CH₃ | CH | C—(A)r—Q | CH | CH | CH₂ | 1 | Q34 |
| 14-381 | CH | C—CH₃ | CH | C—(A)r—Q | CH | CH | CH₂ | 1 | Q34 |
| 14-382 | CH | C—CH₃ | CH | C—(A)r—Q | CH | CH | CH₂ | 1 | Q34 |
| 14-383 | CH | C—CH₃ | CH | C—(A)r—Q | CH | CH | CH₂ | 1 | Q34 |
| 14-384 | CH | C—CH₃ | CH | C—(A)r—Q | CH | CH | CH₂ | 1 | Q34 |
| 14-385 | CH | C—CH₃ | CH | C—(A)r—Q | CH | CH | CH₂ | 1 | Q35 |
| 14-386 | CH | C—CH₃ | CH | C—(A)r—Q | CH | CH | CH₂ | 1 | Q36 |
| 14-387 | CH | C—CH₃ | N | C—(A)r—Q | CH | CH | CH₂ | 1 | Q34 |
| 14-388 | CH | C—CH₃ | CH | C—(A)r—Q | CH | CH | CH₂ | 1 | Q35 |
| 14-389 | CH | C—CH₃ | CH | C—(A)r—Q | CH | CH | CH₂ | 1 | Q39 |
| 14-390 | CH | C—CH₃ | CH | C—(A)r—Q | CH | CH | CH₂ | 1 | Q39 |
| 14-391 | CH | C—CH₃ | CH | C—(A)r—Q | CH | CH | CH₂ | 1 | Q34 |
| 14-392 | CH | C—CH₃ | CH | C—(A)r—Q | CH | CH | CH₂ | 1 | Q34 |
| 14-393 | CH | C—CH₃ | N | C—(A)r—Q | CH | CH | CH₂ | 1 | Q34 |
| 14-394 | CH | C—CH₃ | N | C—(A)r—Q | CH | CH | CH₂ | 1 | Q34 |
| 14-395 | CH | C—CH₃ | CH | C—(A)r—Q | CH | CH | CH₂ | 1 | Q34 |
| 14-396 | CH | C—CH₃ | N | C—(A)r—Q | CH | CH | CH₂ | 1 | Q34 |
| 14-397 | CH | C—CH₃ | N | C—(A)r—Q | CH | CH | CH₂ | 1 | Q34 |
| 14-398 | C—SCH₂CH═CH₂ | C—CH₃ | CH | C—(A)r—Q | CH | CH | CH₂ | 1 | Q34 |
| 14-399 | C—SCH₂CF₃ | C—CH₃ | CH | C—(A)r—Q | CH | CH | CH₂ | 1 | Q34 |
| 14-400 | C—SCF₃ | C—CH₃ | CH | C—(A)r—Q | CH | CH | CH₂ | 1 | Q34 |
| 14-401 | CH | C—Cl | CH | C—(A)r—Q | CH | CH | CH₂ | 1 | Q34 |
| 14-402 | CH | C—CH₃ | CH | C—(A)r—Q | CH | CH | CH₂ | 1 | Q34 |
| 14-403 | CH | C—CH₃ | CH | C—(A)r—Q | CH | CH | CH₂ | 1 | Q34 |
| 14-404 | CH | C—CH₃ | CH | C—(A)r—Q | CH | CH | CH₂ | 1 | Q34 |
| 14-405 | CH | C—CH₃ | CH | C—(A)r—Q | CH | CH | CH₂ | 1 | Q34 |
| 14-406 | CH | C—CH₃ | CH | C—(A)r—Q | CH | CH | CH₂ | 1 | Q34 |
| 14-407 | CH | C—CH₃ | CH | C—(A)r—Q | CH | CH | CH₂ | 1 | Q34 |
| 14-408 | CH | CH | CH | C—(A)r—Q | CH | CH | CH₂ | 1 | Q34 |
| 14-409 | CH | C—CH₃ | CH | C—(A)r—Q | CH | CH | CH₂ | 1 | Q34 |
| 14-410 | CH | C—CH₃ | CH | C—(A)r—Q | CH | CH | CH₂ | 1 | Q34 |
| 14-411 | CH | C—Cl | CH | C—(A)r—Q | CH | CH | CH₂ | 1 | Q34 |
| 14-412 | CH | C—CH₃ | CH | C—(A)r—Q | CH | CH | CH₂ | 1 | Q34 |
| 14-413 | CH | C—Cl | CH | C—(A)r—Q | CH | CH | CH₂ | 1 | Q34 |
| 14-414 | CH | C—CH₃ | CH | C—(A)r—Q | CH | CH | CH₂ | 1 | Q34 |
| 14-415 | CH | C—CH₃ | CH | C—(A)r—Q | N | CH | CH₂ | 1 | Q34 |
| 14-416 | CH | C—CH₃ | CH | C—(A)r—Q | CH | CH | CH₂ | 1 | Q34 |
| 14-417 | CH | C—CH₃ | CH | C—(A)r—Q | CH | CH | CH₂ | 1 | Q34 |
| 14-418 | CH | C—CH₃ | CH | C—(A)r—Q | CH | CH | CH₂ | 1 | Q34 |
| 14-419 | CH | C—CH₃ | CH | C—(A)r—Q | CH | CH | CH₂ | 1 | Q34 |
| 14-420 | CH | C—CH₃ | CH | C—(A)r—Q | CH | CH | CH₂ | 1 | Q34 |
| 14-421 | CH | C—CH₃ | CH | C—(A)r—Q | CH | CH | CH₂ | 1 | Q39 |
| 14-422 | CH | C—CH₃ | CH | C—(A)r—Q | CH | CH | CH₂ | 1 | Q39 |
| 14-423 | CH | C—CH₃ | CH | C—(A)r—Q | CH | CH | CH₂ | 1 | Q39 |
| 14-424 | CH | C—CH₃ | CH | C—(A)r—Q | CH | CH | CH₂ | 1 | Q34 |
| 14-425 | CH | C—CH₃ | CH | C—(A)r—Q | CH | CH | CH₂ | 1 | Q34 |
| 14-426 | CH | C—CH₃ | CH | C—(A)r—Q | CH | CH | CH₂ | 1 | Q34 |
| 14-427 | CH | C—CH₃ | CH | C—(A)r—Q | CH | CH | — | 0 | Q43 |
| 14-428 | CH | C—CH₃ | CH | C—(A)r—Q | CH | CH | CH₂ | 1 | Q34 |
| 14-429 | CH | C—CH₃ | CH | C—(A)r—Q | CH | CH | CH₂ | 1 | Q48 |
| 14-430 | CH | C—CH₃ | CH | C—(A)r—Q | CH | CH | CH₂ | 1 | Q48 |
| 14-431 | CH | C—CH₃ | CH | C—(A)r—Q | CH | CH | CH₂ | 1 | Q48 |
| 14-432 | CH | C—CH₃ | CH | C—(A)r—Q | CH | CH | CH₂ | 1 | Q48 |
| 14-433 | CH | C—CH₃ | CH | C—(A)r—Q | CH | CH | CH₂ | 1 | Q48 |
| 14-434 | CH | C—CH₃ | CH | C—(A)r—Q | CH | CH | CH₂ | 1 | Q48 |
| 14-435 | CH | C—CH₃ | CH | C—(A)r—Q | CH | CH | CH₂ | 1 | Q48 |
| 14-436 | CH | C—CH₃ | CH | C—(A)r—Q | CH | CH | CH₂ | 1 | Q34 |
| 14-437 | N | C—CH₃ | CH | C—(A)r—Q | CH | CH | CH₂ | 1 | Q34 |
| 14-438 | CH | C—CH₃ | CH | C—(A)r—Q | CH | CH | CH₂ | 1 | Q48 |
| 14-439 | CH | C—CH₃ | CH | C—(A)r—Q | CH | CH | CH₂ | 1 | Q34 |
| 14-440 | CH | C—CH₃ | CH | C—(A)r—Q | CH | CH | CH₂ | 1 | Q34 |
| 14-441 | CH | C—CH₃ | CH | C—(A)r—Q | CH | CH | CH₂ | 1 | Q34 |
| 14-442 | CH | C—CH₃ | CH | C—(A)r—Q | CH | CH | CH₂ | 1 | Q34 |
| 14-443 | CH | C—CH₃ | CH | C—(A)r—Q | CH | CH | CH₂ | 1 | Q34 |
| 14-444 | CH | C—CH₃ | CH | C—(A)r—Q | CH | CH | CH₂ | 1 | Q34 |
| 14-445 | CH | C—CO₂CH₃ | CH | C—(A)r—Q | CH | CH | CH₂ | 1 | Q48 |
| 14-446 | CH | C—CO₂CH₃ | CH | C—(A)r—Q | CH | CH | CH₂ | 1 | Q48 |
| 14-447 | CH | C—CONH₂ | CH | C—(A)r—Q | CH | CH | CH₂ | 1 | Q48 |
| 14-448 | CH | C—CONHCH(CH₃)₂ | CH | C—(A)r—Q | CH | CH | CH₂ | 1 | Q48 |
| 14-449 | CH | C—CON(CH₃)₂ | CH | C—(A)r—Q | CH | CH | CH₂ | 1 | Q48 |
| 14-450 | CH | C—CH₃ | CH | C—(A)r—Q | CH | CH | S | 1 | Q37 |
| 14-451 | CH | C—CH₃ | CH | C—(A)r—Q | CH | CH | SO | 1 | Q37 |
| 14-452 | CH | C—CH₃ | CH | C—(A)r—Q | CH | CH | SO₂ | 1 | Q37 |
| 14-453 | CH | C—CH₃ | CH | C—(A)r—Q | CH | CH | S | 1 | Q37 |
| 14-454 | CH | C—CH₃ | CH | C—(A)r—Q | CH | CH | SO | 1 | Q37 |
| 14-455 | CH | C—CH₃ | CH | C—(A)r—Q | CH | CH | SO₂ | 1 | Q37 |
| 14-456 | CH | C—CH₃ | CH | C—(A)r—Q | CH | CH | CH₂ | 1 | Q34 |
| 14-457 | CH | C—CH₃ | CH | C—(A)r—Q | CH | CH | CH₂ | 1 | Q34 |
| 14-458 | CH | C—CH₃ | CH | C—(A)r—Q | CH | CH | CH₂ | 1 | Q34 |
| 14-459 | CH | C—CH₃ | CH | C—(A)r—Q | CH | CH | CH₂ | 1 | Q34 |
| 14-460 | CH | C—CH₃ | CH | C—(A)r—Q | CH | CH | CH₂ | 1 | Q34 |
| 14-461 | CH | C—CH₃ | CH | C—(A)r—Q | CH | CH | CH₂ | 1 | Q34 |
| 14-462 | CH | C—CH₃ | CH | C—(A)r—Q | CH | CH | CH₂ | 1 | Q34 |
| 14-463 | CH | C—CH₃ | CH | C—(A)r—Q | CH | CH | CH₂ | 1 | Q34 |
| 14-464 | CH | C—CH₃ | CH | C—(A)r—Q | CH | CH | CH₂ | 1 | Q34 |
| 14-465 | CH | C—CH₃ | CH | C—(A)r—Q | CH | CH | CH₂ | 1 | Q34 |
| 14-466 | CH | C—CH₃ | CH | C—(A)r—Q | CH | CH | CH₂ | 1 | Q34 |
| 14-467 | CH | C—CH₃ | CH | C—(A)r—Q | CH | CH | CH₂ | 1 | Q34 |
| 14-468 | CH | C—CH₃ | CH | C—(A)r—Q | CH | CH | CH₂ | 1 | Q34 |
| 14-469 | CH | C—CH₃ | CH | C—(A)r—Q | CH | CH | CH₂ | 1 | Q34 |
| 14-470 | CH | C—CH₃ | CH | C—(A)r—Q | CH | CH | CH₂ | 1 | Q34 |
| 14-471 | CH | C—CH₃ | CH | C—(A)r—Q | CH | CH | CH₂ | 1 | Q34 |
| 14-472 | CH | C—CH₃ | CH | C—(A)r—Q | CH | CH | CH₂ | 1 | Q34 |
| 14-473 | CH | C—CH₃ | CH | C—(A)r—Q | CH | CH | CH₂ | 1 | Q34 |
| 14-474 | CH | C—CH₃ | CH | C—(A)r—Q | CH | CH | CH₂ | 1 | Q34 |
| 14-475 | CH | C—CH₃ | CH | C—(A)r—Q | CH | CH | CH₂ | 1 | Q34 |
| 14-476 | CH | C—CH₃ | CH | C—(A)r—Q | CH | CH | CH₂ | 1 | Q34 |
| 14-477 | CH | C—CH₃ | CH | C—(A)r—Q | CH | CH | CH₂ | 1 | Q34 |
| 14-16 | CH | C—CH₃ | CH | C—(A)r—Q | CH | CH | CH₂ | 1 | Q34 |
| 14-17 | CH | C—CH₃ | CH | C—(A)r—Q | CH | CH | CH₂ | 1 | Q34 |
| 14-18 | CH | C—CH₃ | CH | C—(A)r—Q | CH | CH | CH₂ | 1 | Q34 |
| 14-19 | CH | C—CH₃ | CH | C—(A)r—Q | CH | CH | CH₂ | 1 | Q34 |
| 14-20 | CH | C—Br | CH | C—(A)r—Q | CH | CH | CH₂ | 1 | Q34 |
| 14-41 | CH | C—CH₃ | CH | C—(A)r—Q | CH | CH | CH₂ | 1 | Q34 |

| | | | | | Physical |
|---|---|---|---|---|---|
| No. | R⁴ | R⁵ | R⁶ | R⁷ | property |
| 14-1 | CF₃ | CF₃ | — | — | mp 72-74° C. |
| 14-2 | 3- | — | — | — | mp 160-163° C. |
| | (trifluoromethyl)phenyl | | | | |
| 14-3 | 4- | — | — | — | mp 197-198° C. |
| | (trifluoromethyl)phenyl | | | | |
| 14-4 | 3- | — | — | — | mp 174-175° C. |
| | (trifluoromethyl)phenyl | | | | |
| 14-5 | 3,5- | — | — | — | mp 137-140° C. |
| | bis(trifluoromethyl)phenyl | | | | |
| 14-6 | 3,5- | — | — | — | mp 186-188° C. |
| | bis(trifluoromethyl)phenyl | | | | |
| 14-7 | CF₃ | CF₃ | — | — | mp 153-155° C. |
| 14-8 | 4- | — | — | — | mp 158-159° C. |
| | (trifluoromethyl)phenyl | | | | |
| 14-9 | 3,5- | — | — | — | mp 144-150° C. |
| | bis(trifluoromethyl)phenyl | | | | |
| 14-10 | 3- | — | — | — | mp 142-144° C. |
| | (trifluoromethyl)phenyl | | | | |
| 14-11 | 4- | — | — | — | mp 192-195° C. |
| | (trifluoromethyl)phenyl | | | | |
| 14-12 | 3- | — | — | — | mp 123-129° C. |
| | (trifluoromethyl)phenyl | | | | |
| 14-13 | CF₃ | CF₃ | — | — | mp 164-165° C. |
| 14-14 | C₂F₅ | CF₃ | — | — | mp 140-142° C. |
| 14-15 | CF₃ | CF₃ | — | — | mp 158-159° C. |
| 14-21 | CF₃ | CF₂Cl | — | — | NMR |
| 14-22 | CF₃ | C₂F₅ | — | — | NMR |
| 14-23 | CF₃ | CH₂CF₃ | — | — | NMR |
| 14-24 | CF₃ | CF₃ | — | — | NMR |
| 14-25 | CF₃ | 5-(trifluoromethyl)- | — | — | mp 96-100° C. |
| | | pyridin-2-yl | | | |
| 14-26 | CF₃ | 3-chloro-5- | — | — | mp 83-90° C. |
| | | (trifluoromethyl)- | | | |
| | | pyridin-2-yl | | | |
| 14-27 | 3-chloro-5- | CF₃ | — | — | mp 48-57° C. |
| | (trifluoromethyl)- | | | | |
| | pyridin-2-yl | | | | |
| 14-28 | CF₃ | 4-(trifluoromethyl)- | — | — | mp 95-101° C. |
| | | pyridin-3-yl | | | |
| 14-29 | CF₃ | 2,6- | — | — | mp 89-102° C. |
| | | bis(pentafluoroethyl)- | | | |
| | | pyridin-4-yl | | | |
| 14-30 | CF₂Cl | C₂F₅ | — | — | |
| 14-31 | CF₂CHF₂ | CF₂CHF₂ | — | — | logP (acid) 3.33 |
| 14-32 | C₂F₅ | H | — | — | NMR |
| 14-33 | C₂F₅ | H | — | — | NMR |
| 14-34 | C₂F₅ | H | — | — | NMR |
| 14-35 | C₂F₅ | CF₂Cl | — | — | NMR |
| 14-36 | C₂F₅ | CHCl₂ | — | — | NMR |
| 14-37 | C₂F₅ | SCH₃ | — | — | NMR |
| 14-38 | CF₃ | SC₂H5 | — | — | logP (acid) 4.13 |
| 14-39 | CF₃ | SCH(CH₃)₂ | — | — | logP (acid) 4.13 |
| 14-40 | CF₃ | SC₅H₁₁-n | — | — | logP (acid) 4.91 |
| 14-42 | C₂F₅ | SCH₂CF₃ | — | — | NMR |
| 14-43 | C₂F₅ | C₃F₇-n | — | — | NMR |
| 14-44 | C₂F₅ | C(CH₃)₃ | — | — | NMR |
| 14-45 | C₂F₅ | CF₃ | — | — | NMR |
| 14-46 | C₂F₅ | 4-chlorophenyl | — | — | NMR |
| 14-47 | C₂F₅ | 3,5-dichloropyridin-2-yl | — | — | mp 116-121° C. |
| 14-48 | C₂F₅ | 5-chloro-pyridin-2-yl | — | — | NMR |
| 14-49 | C₂F₅ | 5-(trifluoromethyl)-pyridin-2-yl | — | — | NMR |
| 14-50 | C₂F₅ | 3-chloro-pyridin-2-yl | — | — | NMR |
| 14-51 | C₂F₅ | furan-2-yl | — | — | NMR |
| 14-52 | C₂F₅ | thiophen-2-yl | — | — | NMR |
| 14-53 | CF₃ | C₂F₅ | — | — | mp 202-204° C. |
| 14-54 | CF₃ | CF₃ | — | — | NMR |
| 14-55 | CF₃ | 3-chloro-5-(trifluoromethyl)- | — | — | mp 97-100° C. |
| | | pyridin-2-yl | | | |
| 14-56 | CF₃ | 4-(trifluoromethyl)-pyridin-3-yl | — | — | mp 96-100° C. |
| 14-57 | C₂F₅ | CF₂Cl | — | — | NMR |
| 14-58 | C₂F₅ | CF₃ | — | — | NMR |
| 14-59 | C₂F₅ | 4-chlorophenyl | — | — | mp 103-108° C. |
| 14-60 | C₂F₅ | CF₃ | — | — | logP (acid) 2.59 |
| 14-61 | C₂F₅ | CF₃ | — | — | logP (neutral) |
| | | | | | 3.00 |
| 14-62 | CF₃ | C₂F₅ | — | — | mp 143-145° C. |
| 14-63 | CF₃ | CF₃ | — | — | NMR |
| 14-64 | CF₃ | 3-chloro-5-(trifluoromethyl)- | — | — | mp 106-109° C. |
| | | pyridin-2-yl | | | |
| 14-65 | CF₃ | 4-(trifluoromethyl)-pyridin-3-yl | — | — | mp 111-113° C. |
| 14-66 | C₂F₅ | CF₂Cl | — | — | NMR |
| 14-67 | C₂F₅ | CF₃ | — | — | NMR |
| 14-68 | C₂F₅ | 4-chlorophenyl | — | — | NMR |
| 14-69 | CF₃ | CF₂Cl | — | — | NMR |
| 14-70 | CF₃ | C₂F₅ | — | — | mp 74-78° C. |
| 14-71 | CF₃ | CF₃ | — | — | NMR |
| 14-72 | CF₂Cl | C₂F₅ | — | — | |
| 14-73 | CF₂CHF₂ | CF₂CHF₂ | — | — | NMR |
| 14-74 | C₂F₅ | CF₂Cl | — | — | NMR |
| 14-75 | CF₃ | C₂F₅ | — | — | NMR |
| 14-76 | CF₃ | CF₃ | — | — | mp 93-99° C. |
| 14-77 | CF₂CHF₂ | CF₂CHF₂ | — | — | NMR |
| 14-78 | CF₃ | C₂F₅ | — | — | NMR |
| 14-79 | CF₃ | CF₃ | — | — | mp 85-89° C. |
| 14-80 | CF₂CHF₂ | CF₂CHF₂ | — | — | NMR |
| 14-81 | CF₃ | CF₂Cl | — | — | NMR |
| 14-82 | CF₂Cl | CF₃ | — | — | NMR |
| 14-83 | CF₂Cl | CF₃ | — | — | |
| 14-84 | CF₂Cl | CF₃ | — | — | |
| 14-85 | CF₂Cl | CF₃ | — | — | |
| 14-86 | CF₂Cl | CF₃ | — | — | |
| 14-87 | CF₂Cl | CF₃ | — | — | |
| 14-88 | CF₂Cl | CF₃ | — | — | |
| 14-89 | CF₂Cl | CF₃ | — | — | |
| 14-90 | CF₂Cl | CF₃ | — | — | |
| 14-91 | CF₂Cl | CF₃ | — | — | |
| 14-92 | CF₂Cl | CF₃ | — | — | |
| 14-93 | CF₂Cl | CF₃ | — | — | |
| 14-94 | CF₂Cl | CF₃ | — | — | mp 199-201° C. |
| 14-95 | CF₂Cl | CF₃ | — | — | |
| 14-96 | CF₂Cl | CF₃ | — | — | |
| 14-97 | CF₂Cl | CF₂Cl | — | — | NMR |
| 14-98 | CF₂Cl | CF₂Cl | — | — | |
| 14-99 | CF₂Cl | CF₂Cl | — | — | |
| 14-100 | CF₂Cl | CF₂Cl | — | — | |
| 14-101 | CF₂Cl | CF₂Cl | — | — | |
| 14-102 | CF₂Cl | CF₂Cl | — | — | |
| 14-103 | CF₂Cl | CF₂Cl | — | — | |
| 14-104 | CF₂Cl | CF₂Cl | — | — | |
| 14-105 | CF₂Cl | CF₂Cl | — | — | |
| 14-106 | CF₂Cl | CF₂Cl | — | — | |
| 14-107 | CF₂Cl | CF₂Cl | — | — | |
| 14-108 | CF₂Cl | CF₂Cl | — | — | |
| 14-109 | CF₂Cl | CF₂Cl | — | — | mp 210-212° C. |
| 14-110 | CF₂Cl | CF₂Cl | — | — | |
| 14-111 | CF₂Cl | CF₂Cl | — | — | |
| 14-112 | CF₃ | C₂F₅ | — | — | NMR |
| 14-113 | CF₂CHF₂ | CF₂CHF₂ | — | — | mp 140-143° C. |
| 14-114 | C₂F₅ | CF₂Cl | — | — | NMR |
| 14-115 | CF₃ | C₂F₅ | — | — | NMR |
| 14-116 | CF₃ | C₂F₅ | — | — | NMR |
| 14-117 | CF₃ | CF₃ | — | — | NMR |
| 14-118 | CF₃ | C₂F₅ | — | — | NMR |
| 14-119 | C₂F₅ | CF₃ | — | — | NMR |
| 14-120 | C₂F₅ | C₂F₅ | — | — | NMR |
| 14-121 | CF₃ | CF₃ | — | — | NMR |
| 14-122 | CF₃ | C₂F₅ | — | — | NMR |
| 14-123 | C₂F₅ | CF₃ | — | — | NMR |
| 14-124 | C₂F₅ | C₂F₅ | — | — | NMR |
| 14-125 | CF₃ | CF₃ | — | — | NMR |
| 14-126 | CF₃ | C₂F₅ | — | — | NMR |
| 14-127 | C₂F₅ | CF₃ | — | — | NMR |
| 14-128 | C₂F₅ | C₂F₅ | — | — | NMR |
| 14-129 | C₂F₅ | CF₃ | — | — | mp 105-110° C. |
| 14-130 | C₂F₅ | CF₃ | — | — | NMR |
| 14-131 | CF₃ | CF₃ | — | — | mp 184-189° C. |
| 14-132 | C₂F₅ | CF₃ | — | — | mp 203-206° C. |
| 14-133 | C₂F₅ | CF₃ | — | — | NMR |
| 14-134 | C₂F₅ | CF₃ | — | — | mp 83-87° C. |
| 14-135 | C₂F₅ | CF₃ | — | — | NMR |
| 14-136 | C₂F₅ | CF₂Cl | — | — | NMR |
| 14-137 | CF₃ | CF₃ | — | — | NMR |
| 14-138 | C₂F₅ | CF₂Cl | — | — | NMR |
| 14-139 | CF₃ | CF₃ | — | — | NMR |
| 14-140 | CF₃ | CF₃ | — | — | NMR |
| 14-141 | C₂F₅ | CF₂Cl | — | — | NMR |
| 14-142 | CF₃ | CF₃ | — | — | NMR |
| 14-143 | CF₃ | CF₃ | — | — | NMR |
| 14-144 | CF₃ | CF₃ | — | — | NMR |
| 14-145 | CF₃ | CF₃ | — | — | NMR |
| 14-146 | CF₃ | CF₃ | — | — | NMR |
| 14-147 | CF₃ | CF₃ | — | — | NMR |
| 14-148 | CHF₂ | C₂F₅ | — | — | NMR |
| 14-149 | C₂H₅ | C₇F₁₅-n | — | — | NMR |
| 14-150 | H | C₂F₅ | — | — | NMR |
| 14-151 | CH₃ | C₂F₅ | — | — | NMR |
| 14-152 | CHF₂ | C₂F₅ | — | — | NMR |
| 14-153 | C₂H₅ | C₇F₁₅-n | — | — | mp 138-141° C. |
| 14-154 | H | C₂F₅ | — | — | mp 157-161° C. |
| 14-155 | C₂H₅ | C₇F₁₅-n | — | — | NMR |
| 14-156 | H | C₂F₅ | — | — | NMR |
| 14-157 | C₇F₁₅-n | — | — | — | NMR |
| 14-158 | C₃F₇-n | — | — | — | NMR |
| 14-159 | C₂F₅ | — | — | — | NMR |
| 14-160 | C₇F₁₅-n | — | — | — | NMR |
| 14-161 | C₃F₇-n | — | — | — | NMR |
| 14-162 | C₇F₁₅-n | — | — | — | NMR |
| 14-163 | 3,5- | — | — | — | mp 98-102° C. |
| | bis(trifluoromethyl)phenyl | | | | |
| 14-164 | CF₃ | CF₃ | — | — | |
| 14-165 | CF₃ | CF₃ | — | — | NMR |
| 14-166 | CF₃ | CF₃ | — | — | NMR |
| 14-167 | C₂F₅ | CF₃ | — | — | NMR |
| 14-168 | C₂F₅ | CF₃ | — | — | NMR |
| 14-169 | CF₃ | CF₂Cl | — | — | NMR |
| 14-170 | CF₃ | C₂F₅ | — | — | NMR |
| 14-171 | CF₂Cl | C₂F₅ | — | — | |
| 14-172 | CF₂CHF₂ | CF₂CHF₂ | — | — | NMR |
| 14-173 | C₂F₅ | CF₂Cl | — | — | NMR |
| 14-174 | CF₃ | C₂F₅ | — | — | NMR |
| 14-175 | CF₃ | C₂F₅ | — | — | NMR |
| 14-176 | CF₃ | C₂F₅ | — | — | NMR |
| 14-177 | CF₃ | C₂F₅ | — | — | NMR |
| 14-178 | CF₃ | C₂F₅ | — | — | NMR |
| 14-179 | CF₃ | C₂F₅ | — | — | NMR |
| 14-180 | CF₃ | C₂F₅ | — | — | NMR |
| 14-181 | CF₃ | C₂F₅ | — | — | NMR |
| 14-182 | CF₃ | CF₃ | — | — | NMR |
| 14-183 | CF₃ | C₂F₅ | — | — | mp 59° C. |
| 14-184 | C₂F₅ | CF₃ | — | — | NMR |
| 14-185 | C₂F₅ | C₂F₅ | — | — | NMR |
| 14-186 | CF₃ | CF₃ | — | — | NMR |
| 14-187 | CF₃ | C₂F₅ | — | — | NMR |
| 14-188 | C₂F₅ | CF₃ | — | — | NMR |
| 14-189 | C₂F₅ | C₂F₅ | — | — | NMR |
| 14-190 | CF₃ | CF₃ | — | — | NMR |
| 14-191 | CF₃ | C₂F₅ | — | — | NMR |
| 14-192 | C₂F₅ | CF₃ | — | — | NMR |
| 14-193 | C₂F₅ | C₂F₅ | — | — | NMR |
| 14-194 | C₂F₅ | CF₃ | — | — | NMR |
| 14-195 | CF₃ | CF₃ | — | — | |
| 14-196 | CF₃ | CF₃ | — | — | mp 172-174° C. |
| 14-197 | CF₃ | CF₃ | — | — | NMR |
| 14-198 | CF₃ | CF₂Cl | — | — | mp 66-71° C. |
| 14-199 | CF₃ | C₂F₅ | — | — | mp 81-89° C. |
| 14-200 | CF₂CHF₂ | CF₂CHF₂ | — | — | mp 126-131° C. |
| 14-201 | C₂F₅ | CF₂Cl | — | — | NMR |
| 14-202 | CF₃ | C₂F₅ | — | — | NMR |
| 14-203 | C₂F₅ | CF₂Cl | — | — | mp 194-198° C. |
| 14-204 | CF₃ | C₂F₅ | — | — | NMR |
| 14-205 | C₂F₅ | CF₂Cl | — | — | NMR |
| 14-206 | CF₃ | C₂F₅ | — | — | NMR |
| 14-207 | CF₃ | C₂F₅ | — | — | NMR |
| 14-208 | CF₃ | C₂F₅ | — | — | NMR |
| 14-209 | phenyl | CF₃ | — | — | mp 94-96° C. |
| 14-210 | CF₃ | C₂F₅ | — | — | mp 82-87° C. |
| 14-211 | C₂F₅ | CF₂Cl | — | — | NMR |
| 14-212 | 3,5- | CF₃ | — | — | mp 101-106° C. |
| | bis(trifluoromethyl)phenyl | | | | |
| 14-213 | CF₃ | C₂F₅ | — | — | NMR |
| 14-214 | phenyl | CF₃ | — | — | mp 99-103° C. |
| 14-215 | CF₃ | C₂F₅ | — | — | NMR |
| 14-216 | CF₃ | CF₃ | — | — | NMR |
| 14-217 | CF₃ | C₂F₅ | — | — | NMR |
| 14-218 | C₂F₅ | CF₃ | — | — | NMR |
| 14-219 | C₂F₅ | C₂F₅ | — | — | NMR |
| 14-220 | CF₃ | CF₃ | — | — | NMR |
| 14-221 | CF₃ | C₂F₅ | — | — | NMR |
| 14-222 | C₂F₅ | CF₃ | — | — | NMR |
| 14-223 | C₂F₅ | C₂F₅ | — | — | NMR |
| 14-224 | CF₃ | CF₃ | — | — | NMR |
| 14-225 | CF₃ | C₂F₅ | — | — | NMR |
| 14-226 | C₂F₅ | CF₃ | — | — | NMR |
| 14-227 | C₂F₅ | C₂F₅ | — | — | NMR |
| 14-228 | 3,5- | CF₃ | — | — | mp 116-119° C. |
| | bis(trifluoromethyl)phenyl | | | | |
| 14-229 | 3,5- | CF₃ | — | — | mp 104-111° C. |
| | bis(trifluoromethyl)phenyl | | | | |
| 14-230 | 2- | CF₃ | — | — | mp 111-114° C. |
| | chlorophenyl | | | | |
| 14-231 | 2- | CF₃ | — | — | mp 170-175° C. |
| | chlorophenyl | | | | |
| 14-232 | CF₃ | CF₃ | — | — | NMR |
| 14-233 | C₂F₅ | C₂F₅ | — | — | NMR |
| 14-234 | CF₃ | CF₃ | — | — | NMR |
| 14-235 | C₂F₅ | C₂F₅ | — | — | NMR |
| 14-236 | 3,5-bis(trifluoromethyl)phenyl | — | — | — | mp 105-108° C. |
| 14-237 | 3,5-bis(trifluoromethyl)phenyl | — | — | — | mp 119-121° C. |
| 14-238 | 3,5-bis(trifluoromethyl)phenyl | — | — | — | mp 199-202° C. |
| 14-239 | 3-(trifluoromethyl)phenyl | — | — | — | mp 92-98° C. |
| 14-240 | 3,5-bis(trifluoromethyl)phenyl | — | — | — | mp 231-232° C. |
| 14-241 | 3-(trifluoromethyl)phenyl | — | — | — | mp 183-184° C. |
| 14-242 | 3,5-bis(trifluoromethyl)phenyl | — | — | — | mp 141-144° C. |
| 14-243 | 3,5-bis(trifluoromethyl)phenyl | — | — | — | mp 196-199° C. |
| 14-244 | 3,5-bis(trifluoromethyl)phenyl | — | — | — | mp 179-183° C. |
| 14-245 | 3,5-bis(trifluoromethyl)phenyl | — | — | — | mp 103-107° C. |
| 14-246 | 3,5-bis(trifluoromethyl)phenyl | — | — | — | mp 103-105° C. |
| 14-247 | 3,5-bis(trifluoromethyl)phenyl | — | — | — | mp 95-107° C. |
| 14-249 | 3,5- | — | — | — | mp 193-195° C. |
| | bis(trifluoromethyl)phenyl | | | | |
| 14-250 | 3,5- | — | — | — | mp 104-109° C. |
| | bis(trifluoromethyl)phenyl | | | | |
| 14-251 | 3,5- | — | — | — | mp 178-187° C. |
| | bis(trifluoromethyl)phenyl | | | | |
| 14-252 | 4-(trifluoromethyl)phenyl | — | — | — | mp 182-185° C. |
| 14-253 | 3,5- | — | — | — | mp 194-196° C. |
| | bis(trifluoromethyl)phenyl | | | | |
| 14-254 | 3-(trifluoromethyl)phenyl | — | — | — | mp 77-82° C. |
| 14-255 | 4-(trifluoromethyl)phenyl | — | — | — | mp 204-207° C. |
| 14-256 | 3,5- | — | — | — | mp 222-223° C. |
| | bis(trifluoromethyl)phenyl | | | | |
| 14-257 | 3-(trifluoromethyl)phenyl | — | — | — | mp 118-127° C. |
| 14-258 | 3,5- | — | — | — | mp 91-95° C. |
| | bis(trifluoromethyl)phenyl | | | | |
| 14-259 | 3,5- | — | — | — | mp 189-190° C. |
| | bis(trifluoromethyl)phenyl | | | | |
| 14-260 | 3,5- | — | — | — | mp 189-191° C. |
| | bis(trifluoromethyl)phenyl | | | | |
| 14-261 | 3,5- | — | — | — | mp 131-135° C. |
| | bis(trifluoromethyl)phenyl | | | | |
| 14-262 | CF₃ | CF₃ | — | — | mp 139-145° C. |
| 14-263 | C₂F₅ | CF₃ | — | — | mp 144-147° C. |
| 14-264 | C₂F₅ | CF₃ | — | — | mp 201° C. |
| 14-265 | C₂F₅ | CF₃ | — | — | NMR |
| 14-266 | CF₃ | CF₃ | — | — | mp 124-125° C. |
| 14-267 | CF₃ | CF₃ | — | — | NMR |
| 14-268 | CF₃ | CF₃ | — | — | logP (acid) 3.26 |
| 14-269 | CF₃ | CF₃ | — | — | |
| 14-270 | CF₃ | CF₃ | — | — | |
| 14-271 | CF₃ | CF₃ | — | — | logP (acid) 3.37 |
| 14-272 | CF₃ | CF₃ | — | — | |
| 14-273 | CF₃ | CF₃ | — | — | |
| 14-274 | CF₃ | CF₃ | — | — | logP (acid) 3.44 |
| 14-275 | CF₃ | CF₃ | — | — | |
| 14-276 | CF₃ | CF₃ | — | — | |
| 14-277 | C₂F₅ | CF₃ | — | — | |
| 14-278 | C₂F₅ | CF₃ | — | — | |
| 14-279 | C₂F₅ | CF₃ | — | — | |
| 14-280 | C₂F₅ | CF₃ | — | — | |
| 14-281 | C₂F₅ | CF₃ | — | — | |
| 14-282 | C₂F₅ | CF₃ | — | — | |
| 14-283 | C₂F₅ | CF₃ | — | — | |
| 14-284 | C₂F₅ | CF₃ | — | — | |
| 14-285 | C₂F₅ | CF₃ | — | — | |
| 14-286 | C₂F₅ | CF₃ | — | — | mp 110-112° C. |
| 14-287 | C₂F₅ | CF₃ | — | — | mp 108-111° C. |
| 14-288 | C₂F₅ | CF₃ | — | — | mp 92-95° C. |
| 14-289 | C₂F₅ | CF₃ | — | — | mp 198° C. |
| 14-290 | C₂F₅ | CF₃ | — | — | mp 137° C. |
| 14-291 | C₂F₅ | CF₃ | — | — | mp 86° C. |
| 14-292 | C₂F₅ | H | — | — | |
| 14-293 | CF₃ | H | — | — | |
| 14-294 | CF₃ | H | — | — | |
| 14-295 | C₂F₅ | CF₃ | — | — | logP (acid) 3.56 |
| 14-296 | C₂F₅ | CF₃ | — | — | logP (acid) 3.46 |
| 14-297 | C₂F₅ | CF₃ | — | — | logP (acid) 3.70 |
| 14-298 | CF₃ | CF₃ | — | — | logP (acid) 3.08 |
| 14-299 | 4- | — | — | — | NMR |
| | (trifluoromethyl)phenyl | | | | |
| 14-300 | C₂F₅ | CF₃ | — | — | logP (acid) 3.84 |
| 14-301 | CF₃ | CF₃ | — | — | NMR |
| 14-302 | CF₂CHF₂ | H | — | — | logP (acid) 2.48 |
| 14-303 | CF₃ | CF₃ | — | — | NMR |
| 14-304 | CH₂CF₃ | CF₃ | — | — | NMR |
| 14-305 | 4- | — | — | — | logP (acid) 4.08 |
| | (trifluoromethyl)phenyl | | | | |
| 14-306 | CF₃ | — | — | — | logP (acid) 2.91 |
| 14-307 | C₃F₇-n | — | — | — | logP (acid) 3.71 |
| 14-308 | C₂F₅ | CF₃ | — | — | NMR |
| 14-309 | CF₂CHF₂ | H | — | — | logP (acid) 2.38 |
| 14-310 | C₃F₇-n | H | — | — | logP (acid) 3.16 |
| 14-311 | CF₂CHF₂ | CF₃ | — | — | logP (acid) 3.45 |
| 14-312 | CF₂CHF₂ | CF₃ | — | — | |
| 14-313 | CF₃ | CF₂CHF₂ | — | — | |
| 14-314 | H | H | — | — | NMR |
| 14-315 | H | H | — | — | NMR |
| 14-316 | C₂F₅ | CH₃ | — | — | NMR |
| 14-317 | CH₃ | CF₃ | — | — | |
| 14-318 | CH₃ | CF₃ | — | — | |
| 14-319 | CH₃ | CF₃ | — | — | |
| 14-320 | CH₃ | CF₃ | — | — | logP (neutral) |
| | | | | | 2.16 |
| 14-321 | CH₃ | CF₃ | — | — | logP (acid) 1.37 |
| 14-322 | CH₃ | CF₃ | — | — | logP (acid) 1.60 |
| 14-323 | cyclopropyl | CF₃ | — | — | logP (acid) 2.57 |
| 14-324 | CHF₂ | CF₃ | — | — | |
| 14-325 | CHF₂ | CF₃ | — | — | |
| 14-326 | CHF₂ | CF₃ | — | — | |
| 14-327 | CHF₂ | CF₃ | — | — | |
| 14-328 | CHF₂ | C₂F₅ | — | — | logP (acid) 3.68 |
| 14-329 | CHF₂ | C₂F₅ | — | — | logP (acid) 2.5 |
| 14-330 | CHF₂ | C₂F₅ | — | — | logP (acid) 2.85 |
| 14-331 | CHF₂ | C₂F₅ | — | — | logP (acid) 3.75 |
| 14-332 | CHF₂ | C₂F₅ | — | — | |
| 14-333 | CHF₂ | C₂F₅ | — | — | |
| 14-334 | CHF₂ | C₂F₅ | — | — | logP (acid) 3.11 |
| 14-335 | CHF₂ | C₂F₅ | — | — | |
| 14-336 | CHF₂ | C₂F₅ | — | — | |
| 14-337 | CF₃ | CF₃ | — | — | NMR |
| 14-338 | CF₃ | CF₃ | — | — | NMR |
| 14-339 | CF₃ | CF₂CHF₂ | — | — | NMR |
| 14-340 | CF₂CHF₂ | CF₃ | — | — | NMR |
| 14-341 | CF₃ | CF₃ | — | — | NMR |
| 14-342 | CF₃ | CF₃ | — | — | NMR |
| 14-343 | CF₂CHF₂ | CF₃ | — | — | |
| 14-344 | CF₃ | CF₂CHF₂ | — | — | |
| 14-345 | C₂F₅ | C₂F₅ | — | — | NMR |
| 14-346 | C₂F₅ | CF₃ | — | — | NMR |
| 14-347 | C₂F₅ | CF₃ | — | — | NMR |
| 14-348 | 4- | — | — | — | logP (acid) 3.22 |
| | (trifluoromethyl)phenyl | | | | |
| 14-349 | 4- | — | — | — | logP (acid) 3.23 |
| | (trifluoromethyl)phenyl | | | | |
| 14-350 | C₃F₇-n | H | — | — | logP (acid) 2.41 |
| 14-351 | CF₂CHF₂ | CF₃ | — | — | |
| 14-352 | CF₃ | CF₂CHF₂ | — | — | |
| 14-353 | CF₃ | NHCOCF₃ | — | — | logP (acid) 3.11 |
| 14-354 | C₂F₅ | C₂F₅ | — | — | NMR |
| 14-355 | C₂F₅ | CF₃ | — | — | NMR |
| 14-356 | C₂F₅ | CF₃ | — | — | NMR |
| 14-357 | C₂F₅ | CF₃ | — | — | logP (acid) 3.79 |
| 14-358 | C₂F₅ | CF₃ | — | — | logP (acid) 3.95 |
| 14-359 | C₂F₅ | CF₃ | — | — | logP (acid) 4.06 |
| 14-360 | C₂F₅ | CF₃ | — | — | logP (acid) 3.86 |
| 14-361 | C₂F₅ | CF₃ | — | — | logP (acid) 3.95 |
| 14-362 | C₂F₅ | CF₃ | — | — | logP (acid) 3.92 |
| 14-363 | C₂F₅ | CF₃ | — | — | logP (acid) 4.03 |
| 14-364 | CF₃ | CF₃ | — | — | logP (acid) 2.98 |
| 14-365 | CF₃ | CF₃ | — | — | logP (acid) 3.30 |
| 14-366 | CF₃ | CF₃ | — | — | logP (acid) 2.07 |
| 14-367 | CF₃ | CF₃ | — | — | logP (acid) 2.25 |
| 14-368 | CF₃ | CF₃ | — | — | logP (acid) 2.43 |
| 14-369 | CF₃ | CF₃ | — | — | logP (acid) 2.72 |
| 14-370 | CF₃ | CF₃ | — | — | mp 184-186° C. |
| 14-371 | CF₃ | CF₃ | — | — | mp 109-110° C. |
| 14-372 | CF₃ | CF₃ | — | — | mp 199-200° C. |
| 14-373 | CF₃ | CF₃ | — | — | NMR |
| 14-374 | CF₃ | CF₃ | — | — | NMR |
| 14-375 | CF₃ | CF₃ | — | — | NMR |
| 14-376 | CF₃ | CF₂Cl | — | — | NMR |
| 14-377 | CF₃ | CF₂Cl | — | — | |
| 14-378 | C₂F₅ | CF₃ | — | — | mp 111-112° C. |
| 14-379 | C₂F₅ | CF₃ | — | — | mp 204-206° C. |
| 14-380 | C₂F₅ | CF₂Cl | — | — | NMR |
| 14-381 | C₂F₅ | CF₂Cl | — | — | |
| 14-382 | CF₃ | CF₃ | — | — | NMR |
| 14-383 | CF₂CHF₂ | CF₂CHF₂ | — | — | NMR |
| 14-384 | C₂F₅ | CF₃ | — | — | logP (acid) 3.88 |
| 14-385 | CHF₂ | C₂F₅ | — | — | NMR |
| 14-386 | CHF₂ | C₂F₅ | — | — | NMR |
| 14-387 | C₂F₅ | C₂F₅ | — | — | logP (acid) 4.33 |
| 14-388 | CHF₂ | C₂F₅ | — | — | NMR |
| 14-389 | CHF₂ | C₂F₅ | — | — | logP (acid) 3.53 |
| 14-390 | CHF₂ | C₂F₅ | — | — | |
| 14-391 | C₂F₅ | CF₃ | — | — | mp 208-209° C. |
| 14-392 | C₂F₅ | CF₃ | — | — | NMR |
| 14-393 | C₂F₅ | CF₃ | — | — | logP (acid) 3.34 |
| 14-394 | C₂F₅ | CF₃ | — | — | logP (acid) 2.39 |
| 14-395 | C₂F₅ | CF₃ | — | — | logP (acid) 3.13 |
| 14-396 | C₂F₅ | CF₃ | — | — | logP (acid) 2.76 |
| 14-397 | CF₂CHF₂ | CF₃ | — | — | logP (acid) 3.49 |
| 14-398 | CF₃ | CF₃ | — | — | mp 166-168° C. |
| 14-399 | CF₃ | CF₃ | — | — | NMR |
| 14-400 | CF₃ | CF₃ | — | — | NMR |
| 14-401 | CF₃ | CF₃ | — | — | NMR |
| 14-402 | CF₃ | CF₃ | — | — | mp 101-103° C. |
| 14-403 | CF₃ | CF₃ | — | — | NMR |
| 14-404 | CF₃ | CF₃ | — | — | NMR |
| 14-405 | CF₃ | CF₃ | — | — | mp 126-128° C. |
| 14-406 | CF₃ | CF₃ | — | — | NMR |
| 14-407 | CF₃ | CF₃ | — | — | NMR |
| 14-408 | CF₃ | CF₃ | — | — | mp 178-179° C. |
| 14-409 | CF₃ | CF₃ | — | — | NMR |
| 14-410 | CF₃ | CF₃ | — | — | NMR |
| 14-411 | CF₃ | CF₃ | — | — | NMR |
| 14-412 | CF₃ | CF₃ | — | — | mp 185-186° C. |
| 14-413 | CF₃ | CF₃ | — | — | NMR |
| 14-414 | CF₃ | CF₃ | — | — | logP (acid) 3.54 |
| 14-415 | CF₃ | CF₃ | — | — | logP (acid) 3.3 |
| 14-416 | CF₃ | CF₃ | — | — | NMR |
| 14-417 | CF₃ | CF₃ | — | — | mp 188-190° C. |
| 14-418 | CF₃ | CHF₂ | — | — | mp 176-177° C. |
| 14-419 | C₂F₅ | H | — | — | mp 116-121° C. |
| 14-420 | CF₃ | H | — | — | mp 134-137° C. |
| 14-421 | NH₂ | C₂F₅ | — | — | logP (acid) 2.79 |
| 14-422 | NH₂ | C₂F₅ | — | — | logP (acid) 1.82 |
| 14-423 | NH₂ | C₂F₅ | — | — | logP (acid) 2.13 |
| 14-424 | C₂F₅ | Br | — | — | NMR |
| 14-425 | C₂F₅ | Br | — | — | NMR |
| 14-426 | C₂F₅ | C₂F₅ | — | — | mp 196-197° C. |
| 14-427 | 3,5- | — | — | — | mp 187-190° C. |
| | bis(trifluoromethyl)phenyl | | | | |
| 14-428 | CF₃ | CF₃ | — | — | |
| 14-429 | 3,5- | — | — | — | mp 131-140° C. |
| | bis(trifluoromethyl)phenyl | | | | |
| 14-430 | 3,5- | — | — | — | |
| | bis(trifluoromethyl)phenyl | | | | |
| 14-431 | 3,5- | — | — | — | |
| | bis(trifluoromethyl)phenyl | | | | |
| 14-432 | 3,5- | — | — | — | mp 109-114° C. |
| | bis(trifluoromethyl)phenyl | | | | |
| 14-433 | 3,5- | — | — | — | |
| | bis(trifluoromethyl)phenyl | | | | |
| 14-434 | 3,5- | — | — | — | |
| | bis(trifluoromethyl)phenyl | | | | |
| 14-435 | 3,5- | — | — | — | |
| | bis(trifluoromethyl)phenyl | | | | |
| 14-436 | CF₃ | CF₃ | — | — | |
| 14-437 | CF₃ | CF₃ | — | — | |
| 14-438 | 3,5- | — | — | — | mp 187-190° C. |
| | bis(trifluoromethyl)phenyl | | | | |
| 14-439 | CF₃ | CF₃ | — | — | |
| 14-440 | CF₃ | CF₃ | — | — | |
| 14-441 | CF₃ | CF₃ | — | — | |
| 14-442 | CF₃ | CF₃ | — | — | |
| 14-443 | CF₃ | CF₃ | — | — | |
| 14-444 | CF₃ | CF₃ | — | — | |
| 14-445 | 3,5- | — | — | — | NMR |
| | bis(trifluoromethyl)phenyl | | | | |
| 14-446 | 3,5- | — | — | — | NMR |
| | bis(trifluoromethyl)phenyl | | | | |
| 14-447 | 3,5- | — | — | — | |
| | bis(trifluoromethyl)phenyl | | | | |
| 14-448 | 3,5- | — | — | — | NMR |
| | bis(trifluoromethyl)phenyl | | | | |
| 14-449 | 3,5- | — | — | — | |
| | bis(trifluoromethyl)phenyl | | | | |
| 14-450 | CH₃ | C₂F₅ | — | — | |
| 14-451 | CH₃ | C₂F₅ | — | — | |
| 14-452 | CH₃ | C₂F₅ | — | — | |
| 14-453 | CH₃ | C₂F₅ | — | — | |
| 14-454 | CH₃ | C₂F₅ | — | — | |
| 14-455 | CH₃ | C₂F₅ | — | — | |
| 14-456 | CF₃ | CF₃ | — | — | |
| 14-457 | CF₃ | CF₃ | — | — | |
| 14-458 | CF₃ | CF₃ | — | — | |
| 14-459 | CF₃ | CF₃ | — | — | |
| 14-460 | CF₃ | CF₃ | — | — | |
| 14-461 | CF₃ | CF₃ | — | — | |
| 14-462 | CF₃ | CF₃ | — | — | |
| 14-463 | CF₃ | CF₃ | — | — | |
| 14-464 | CF₃ | CF₃ | — | — | |
| 14-465 | CF₃ | CF₃ | — | — | |
| 14-466 | CF₃ | CF₃ | — | — | |
| 14-467 | C₂F₅ | 2-cyanophenyl | — | — | |
| 14-468 | C₂F₅ | 3-methylthiophenyl | — | — | |
| 14-469 | C₂F₅ | 3- | — | — | |
| | | (trifluoromethylthio)phenyl | | | |
| 14-470 | C₂F₅ | 4-methylphenyl | — | — | |
| 14-471 | C₂F₅ | 4-ethylphenyl | — | — | |
| 14-472 | C₂F₅ | 4-vinylphenyl | — | — | |
| 14-473 | C₂F₅ | 4-methoxyphenyl | — | — | |
| 14-474 | C₂F₅ | 4-acety | — | — | |
| | | phenyl | | | |
| 14-475 | C₂F₅ | 4-methoxylcarbonyl | — | — | |
| | | phenyl | | | |
| 14-476 | C₂F₅ | 4-(trifluoromethoxy)phenyl | — | — | |
| 14-477 | CF₃ | NHCOCH₃ | — | — | |
| 14-16 | C₂F₅ | CF₃ | — | — | mp 149-150° C. |
| 14-17 | C₂F₅ | CF₃ | — | — | NMR |
| 14-18 | CF₃ | CF₃ | — | — | mp 178-179° C. |
| 14-19 | CF₃ | CF₃ | — | — | mp 167-169° C. |
| 14-20 | CF₃ | CF₃ | — | — | NMR |
| 14-41 | C₂F₅ | CF₂CF₂Cl | — | — | NMR |

TABLE 15

| No. | R¹ | R² | R³ | W¹ | W² | W³ | W⁴ | W⁵ | W⁶ | W⁷ |
|---|---|---|---|---|---|---|---|---|---|---|
| 15-1 | CH(CH₃)CH₂SCH₃ (S)-isomer | H | H | C—Cl | CH | CH | CH | C—CH₃ | CH | C-(A)r-Q |
| 15-2 | CH(CH₃)CH₂SCH₃ (S)-isomer | H | H | C—Cl | CH | CH | CH | C—CH₃ | CH | C-(A)r-Q |
| 15-3 | CH(CH₃)CH₂SCH₃ (S)-isomer | H | H | C—Cl | CH | CH | CH | C—CH₃ | CH | C-(A)r-Q |
| 15-4 | CH(CH₃)CH₂SCH₃ (S)-isomer | H | H | C—Cl | CH | CH | CH | C—CH₃ | CH | C-(A)r-Q |
| 15-5 | CH(CH₃)CH₂SCH₃ (S)-isomer | H | H | C—Cl | CH | CH | CH | C—CH₃ | CH | C-(A)r-Q |
| 15-6 | CH(CH₃)CH₂SCH₃ (S)-isomer | H | H | C—Cl | CH | CH | CH | C—CH₃ | CH | C-(A)r-Q |
| 15-7 | CH(CH₃)CH₂SCH₃ (S)-isomer | H | H | C—Cl | CH | CH | CH | C—CH₃ | CH | C-(A)r-Q |
| 15-8 | CH(CH₃)CH₂SOCH₃ (S)-isomer | H | H | C—Cl | CH | CH | CH | C—CH₃ | CH | C-(A)r-Q |
| 15-9 | CH(CH₃)CH₂SO₂CH₃ (S)-isomer | H | H | C—Cl | CH | CH | CH | C—CH₃ | CH | C-(A)r-Q |
| 15-10 | CH(CH₃)CH₂SCH₃ (S)-isomer | H | H | C—I | CH | CH | CH | C—CH₃ | CH | C-(A)r-Q |
| 15-11 | CH(CH₃)CH₂SCH₃ (S)-isomer | H | H | C—SCH₃ | CH | CH | CH | C—CH₃ | CH | C-(A)r-Q |
| 15-12 | CH(CH₃)CH₂SCH₃ (S)-isomer | H | H | C—SO₂CH₃ | CH | CH | CH | C—CH₃ | CH | C-(A)r-Q |
| 15-13 | CH(CH₃)CH₂SCH₃ (S)-isomer | H | H | C—Cl | CH | CH | CH | C—CH₃ | CH | C-(A)r-Q |
| 15-14 | CH(CH₃)CH₂SCH₃ (S)-isomer | H | H | C—Cl | CH | CH | CH | C—CH₃ | CH | C-(A)r-Q |
| 15-15 | CH(CH₃)CH₂SOCH₃ (S)-isomer | H | H | C—Cl | CH | CH | CH | C—CH₃ | CH | C-(A)r-Q |
| 15-16 | CH(CH₃)CH₂SO₂CH₃ (S)-isomer | H | H | C—Cl | CH | CH | CH | C—CH₃ | CH | C-(A)r-Q |
| 15-17 | CH(CH₃)CH₂SO₂CH₃ (S)-isomer | H | H | C—Cl | CH | CH | CH | C—CH₃ | CH | C-(A)r-Q |
| 15-18 | CH(CH₃)CH₂SO₂CH₃ (S)-isomer | H | H | C—Cl | CH | CH | CH | C—CH₃ | CH | C-(A)r-Q |
| 15-19 | CH(CH₃)CH₂SO₂CH₃ (S)-isomer | H | H | C—Cl | CH | CH | CH | C—CH₃ | CH | C-(A)r-Q |
| 15-20 | CH(CH₃)CH₂SCH₃ (S)-isomer | H | H | C—Cl | CH | CH | CH | C—CH₃ | CH | C-(A)r-Q |
| 15-21 | CH(CH₃)CH₂SCH₃ (S)-isomer | H | H | C—Cl | CH | CH | CH | C—CH₃ | CH | C-(A)r-Q |
| 15-22 | CH(CH₃)CH₂SCH₃ (S)-isomer | H | H | C—Cl | CH | CH | CH | C—CH₃ | CH | C-(A)r-Q |

| | | | | | | | | | | Physical |
|---|---|---|---|---|---|---|---|---|---|---|
| No. | W⁸ | W⁹ | A | r | Q | R⁴ | R⁵ | R⁶ | R⁷ | property |
| 15-1 | CH | CH | CH₂ | 1 | Q51 | OCH₂CF₃ | H | CF₃ | — | NMR |
| 15-2 | CH | CH | CH₂ | 1 | Q51 | CF₃ | H | CF₃ | — | NMR |
| 15-3 | CH | CH | CH(CN) | 1 | Q51 | CF₃ | H | CF₃ | — | NMR |
| 15-4 | CH | CH | CH(CO₂CH₃) | 1 | Q51 | CF₃ | H | CF₃ | — | NMR |
| 15-5 | CH | CH | CH₂ | 1 | Q51 | C₃F₇-n | H | CF₃ | — | NMR |
| 15-6 | CH | CH | CH(CN) | 1 | Q51 | C₃F₇-n | H | CF₃ | — | NMR |
| 15-7 | CH | CH | CH₂ | 1 | Q51 | C₂F₅ | H | CF₃ | — | NMR |
| 15-8 | CH | CH | CH₂ | 1 | Q51 | C₂F₅ | H | CF₃ | — | NMR |
| 15-9 | CH | CH | CH₂ | 1 | Q51 | C₂F₅ | H | CF₃ | — | NMR |
| 15-10 | CH | CH | CH₂ | 1 | Q51 | C₂F₅ | H | CF₃ | — | |
| 15-11 | CH | CH | CH₂ | 1 | Q51 | C₂F₅ | H | CF₃ | — | |
| 15-12 | CH | CH | CH₂ | 1 | Q51 | C₂F₅ | H | CF₃ | — | |
| 15-13 | CH | CH | CH₂ | 1 | Q51 | C₂F₅ | H | C₂F₅ | — | NMR |
| 15-14 | CH | CH | CH(CN) | 1 | Q51 | C₂F₅ | H | C₂F₅ | — | NMR |
| 15-15 | CH | CH | CH(CN) | 1 | Q51 | CF₃ | H | CF₃ | — | mp 102-106° C. |
| 15-16 | CH | CH | CH₂ | 1 | Q51 | CF₃ | H | CF₃ | — | NMR |
| 15-17 | CH | CH | CH(CN) | 1 | Q51 | CF₃ | H | CF₃ | — | mp 103-108° C. |
| 15-18 | CH | CH | CH(CO₂CH₃) | 1 | Q51 | CF₃ | H | CF₃ | — | mp 102-106° C. |
| 15-19 | CH | CH | CH₂ | 1 | Q51 | C₂F₅ | H | C₂F₅ | — | NMR |
| 15-20 | CH | CH | CH₂ | 1 | Q57 | C₂F₅ | CH₃ | — | — | NMR |
| 15-21 | CH | CH | CH₂ | 1 | Q51 | OCH₃ | H | CF₃ | — | |
| 15-22 | CH | CH | CH₂ | 1 | Q51 | OCH₂CH₃ | H | CF₃ | — | |

TABLE 16

| No. | R¹ | R² | R³ | W¹ | W² | W³ | W⁴ | W⁵ | W⁶ | W⁷ |
|---|---|---|---|---|---|---|---|---|---|---|
| 16-1 | CH(CH₃)CH₂SCH₃ (S)-isomer | H | H | C—Cl | CH | CH | CH | C—CH₃ | CH | C-(A)r-Q |
| 16-2 | CH(CH₃)CH₂SCH₃ (S)-isomer | H | H | C—I | CH | CH | CH | C—CH₃ | CH | C-(A)r-Q |
| 16-3 | CH(CH₃)CH₂SCH₃ (S)-isomer | H | H | C—SCH₃ | CH | CH | CH | C—CH₃ | CH | C-(A)r-Q |
| 16-4 | CH(CH₃)CH₂SCH₃ (S)-isomer | H | H | C—Cl | CH | CH | CH | C—CH₃ | CH | C-(A)r-Q |
| 16-5 | CH(CH₃)CH₂SCH₃ (S)-isomer | H | H | C—Cl | CH | CH | CH | C—CH₃ | CH | C-(A)r-Q |
| 16-6 | CH(CH₃)CH₂SCH₃ (S)-isomer | H | H | C—Cl | CH | CH | CH | C—CH₃ | CH | C-(A)r-Q |
| 16-7 | CH(CH₃)CH₂SCH₃ (S)-isomer | H | H | C—Cl | CH | CH | CH | C—CH₃ | CH | C-(A)r-Q |
| 16-8 | CH(CH₃)CH₂SCH₃ (S)-isomer | H | H | C—Br | CH | CH | CH | C—CH₃ | CH | C-(A)r-Q |
| 16-9 | CH(CH₃)CH₂SCH₃ (S)-isomer | H | H | C—Cl | CH | CH | CH | C—CH₃ | CH | C-(A)r-Q |
| 16-10 | CH(CH₃)CH₂SCH₃ (S)-isomer | H | H | C—SCH₃ | CH | CH | CH | C—CH₃ | CH | C-(A)r-Q |
| 16-11 | CH(CH₃)CH₂SCH₃ (S)-isomer | H | H | C—SO₂CH₃ | CH | CH | CH | C—CH₃ | CH | C-(A)r-Q |
| 16-12 | CH(CH₃)CH₂SCH₃ (S)-isomer | H | H | C—SCH₃ | CH | CH | CH | C—CH₃ | CH | C-(A)r-Q |
| 16-13 | CH(CH₃)CH₂SCH₃ (S)-isomer | H | H | C—SCH₃ | CH | CH | CH | C—CH₃ | CH | C-(A)r-Q |
| 16-14 | CH(CH₃)CH₂SCH₃ (S)-isomer | H | H | C—SO₂CH₃ | CH | CH | CH | C—CH₃ | CH | C-(A)r-Q |

| | | | | | | | | | | Physical |
|---|---|---|---|---|---|---|---|---|---|---|
| No. | W⁸ | W⁹ | A | r | Q | R⁴ | R⁵ | R⁶ | R⁷ | property |
| 16-1 | CH | CH | CH₂ | 1 | Q63 | CF₃ | H | CF₃ | H | |
| 16-2 | CH | CH | CH₂ | 1 | Q63 | CF₃ | H | CF₃ | H | |
| 16-3 | CH | CH | CH₂ | 1 | Q63 | CF₃ | H | CF₃ | H | |
| 16-4 | CH | CH | CH₂ | 1 | Q63 | C₂F₅ | H | CF₃ | H | NMR |
| 16-5 | CH | CH | CH₂ | 1 | Q63 | C₂F₅ | CO₂CH₃ | CF₃ | H | NMR |
| 16-6 | CH | CH | CH₂ | 1 | Q63 | C₂F₅ | COCH₃ | CF₃ | H | NMR |
| 16-7 | CH | CH | CH₂ | 1 | Q63 | CF₃ | H | CH₃ | CH₃ | |
| 16-8 | CH | CH | CH₂ | 1 | Q63 | CF₃ | H | CH₃ | CH₃ | |
| 16-9 | CH | CH | CH₂ | 1 | Q63 | C₂F₅ | H | CH₃ | CH₃ | NMR |
| 16-10 | CH | CH | CH₂ | 1 | Q63 | C₂F₅ | H | CH₃ | CH₃ | |
| 16-11 | CH | CH | CH₂ | 1 | Q63 | C₂F₅ | H | CH₃ | CH₃ | |
| 16-12 | CH | CH | CH₂ | 1 | Q63 | C₂F₅ | COCH₃ | CF₃ | H | NMR |
| 16-13 | CH | CH | CH₂ | 1 | Q63 | C₂F₅ | H | CF₃ | H | NMR |
| 16-14 | CH | CH | CH₂ | 1 | Q63 | C₂F₅ | H | CF₃ | H | NMR |

TABLE 17

| | | |
|---|---|---|
| 1-1 | ¹H-NMR (CDCl₃, δ ppm): | 1.05 (3H, t), 1.11 (3H, t), 2.31 (3H, s), 3.09-3.21 (2H, m), 3.38-3.76 (2H, m), 5.42 (2H, s), 6.90 (1H, s), 7.07-7.14 (2H, m), 7.44 (1H, dd), 7.55 (1H, d), 7.80 (1H, d), 8.04 |
| | | (1H, d), 8.57 (1H, bs) |
| 1-5 | ¹H-NMR | 1.05 (3H, t), 1.11 (3H, t), 2.32 (3H, s), 3.07-3.20 (2H, m), 3.36-3.70 (2H, m), 5.46 (2H, s), 7.14-7.21 (2H, m), 7.45 (1H, dd) 7.56 (1H, d), |
| | (CDCl₃, δ ppm): | 7.81 (1H, d), 8.11 (1H, d), 8.63 (1H, bs) |
| 1-6 | ¹H-NMR | 1.05 (3H, t), 1.10 (3H, t), 2.32 (3H, s), 3.04-3.21 (2H, m), 3.36-3.71 (2H, m), 5.49 (2H, s), 7.13-7.19 (2H, m), 7.45 (1H, dd) 7.56 (1H, d), |
| | (CDCl₃, δ ppm): | 7.80 (1H, d), 8.11 (1H, d), 8.62 (1H, bs) |
| 1-7 | ¹H-NMR | 0.98-1.2 (6H, m), 2.3 (3H, s), 2.7 (3H/2, s), 2.9 (3H/2, s), 3.6-3.7 (1H/2, m), 4.9-5.0 (1H/2, m), 5.4 (2H, s), 6.9 (1H, s), 7.1-8.0 (6H, m), 8.5 (1H, d) |
| | (CDCl₃, δ ppm): | |
| 1-9 | ¹H-NMR | 0.98-1.2 (6H, m), 2.3 (3H, s). 2.7 (3H/2, s), 2.9 (3H/2, s), 3.6-3.7 (1H/2, m), 4.9-5.0 (1H/2, m), 5.5 (2H, s), 7.2-8.1 (6H, m), 8.6 (1H, d) |
| | (CDCl₃, δ ppm): | |
| 1-10 | ¹H-NMR | 1.27 (3H, d), 2.13 (3H, s), 2.25-2.46 (5H, m), 2.69 (3H, s), 4.48-5.04 (1H, m), 5.41 (2H, s), 6.91 (1H, s), 7.03-7.19 (2H, m), 7.38-7.62 (2H, m), |
| | (CDCl₃, δ ppm): | 7.69-8.22 (2H, m), 8.43 (1H, bs) |
| 1-17 | ¹H-NMR | 1.27 (3H, d), 2.04 (3H, s), 2.29-2.53 (5H, m), 2.70 (3H, s), 4.80-5.05 (1H, m), 5.46 (2H, s), 7.10-7.22 (2H, m), 7.40-7.49 (1H, m), 7.57 (1H, d), |
| | (CDCl₃, δ ppm): | 7.72-7.91 (1H, m), 8.09-8.31 (1H, m), 8.74 (1H, bs) |
| 1-29 | ¹H-NMR | 2.31 (3H, s), 3.62 (4H, bs), 3.70 5.42 (2H, s), 6.91 (1H, s), 7.14 (1H, s), 7.15 (1H, d), 7.21 (1H, d), 7.41 (1H, dd), 7.50 (1H, d), 7.60 (1H, bs), |
| | (CDCl₃, δ ppm): | 7.91 (1H, d) |
| 2-13 | ¹H-NMR | 1.29-1.34 (3H, m), 2.03-2.39 (6H, m), 2.63-2.82 (2H, m), 3.15-3.34 (3H, m), 4.35-4.40 (1H, m), 5.22-5.32 (2H, m), 6.71-7.74 (8H, m) |
| | (CDCl₃, δ ppm): | |
| 2-14 | ¹H-NMR | 1.24-1.43 (3H, m), 1.95-2.93 (8H, m), 3.12-3.32 (3H, m), 4.27-4.44 (1H, m), 5.22-5.36 (2H, m), 6.27-7.61 (8H, m) |
| | (CDCl₃, δ ppm): | |
| 2-15 | ¹H-NMR | 0.97-1.21 (3H, m), 1.32-1.41 (3H, m), 2.19-2.22 (3H, m), 2.35-2.38 (3H, m), 2.64-4.41 (5H, m), 5.39-5.52 (2H, m), 6.19-6.21 (1H, m), |
| | (CDCl₃, δ ppm): | 6.64-7.54 (6H, m) |
| 4-4 | ¹H-NMR | 1.29 (3H, d), 1.98 (3H, s), 2.28 (3H, s), 2.63 (2H, m), 4.37 (1H, m), 5.57 (2H, s), 6.20 (1H, d), 6.46 (1H, d), 6.97 (1H, s), 7.17 (1H, t), |
| | (CDCl₃, δ ppm): | 7.43-7.61 (2H, m), 7.77 (1H, d), 7.86 (1H, d), 8.44 (1H, s). |
| 4-5 | ¹H-NMR | 1.27 (3H, d), 1.98 (3H, s), 2.24 (3H, s), 2.54-2.69 (2H, m), 4.28-4.41 (1H, m), 5.44 (2H, s), 6.17 (1H, d), 7.09 (1H, d), 7.25-7.65 (8H, m), |
| | (CDCl₃, δ ppm): | 7.77 (1H, d), 7.94 (1H, d), 8.44 (1H, bs) |
| 4-7 | ¹H-NMR | 1.27 (3H, d), 1.99 (3H, s), 2.19 (3H, s), 2.59 (1H, dd), 2.66 (1H, dd), 4.23-4.41 (1H, m), 5.39 (2H, s), 6.16 (1H, d), 7.03 (1H, d), 7.26-7.32 (2H, |
| | (CDCl₃, δ ppm): | m), 7.45 (1H, dd), 7.56 (1H, d), 7.76 (1H, d), 7.97 (1H, d), 8.44 (1H, bs) |
| 4-8 | ¹H-NMR | 1.26 (3H, d), 1.97 (3H, s), 2.33 (3H, s), 2.56-2.69 (2H, m), 4.31-4.40 (1H, m), 5.59 (2H, s), 6.23 (1H, d), 6.81 (1H, d), 7.23 (1H, m), |
| | (CDCl₃, δ ppm): | 7.43-7.56 (2H, m), 7.76 (1H, d), 7.92 (1H, d), 8.53 (1H, s). |
| 4-12 | ¹H-NMR | 1.27 (3H, d), 2.00 (3H, s), 2.24 (3H, s), 2.53-2.72 (2H, m), 4.26-4.40 (1H, m), 5.50 (2H, s), 5.70 (2H, s), 6.46 (1H, d), 6.58 (1H, d), 6.95 (1H, s), |
| | (CDCl₃, δ ppm): | 7.13 (1H, dd), 7.41 (1H, dd), 7.50 (1H, d), 7.67 (1H, d), 7.78 (1H, d), 8.67 (1H, bs) |
| 4-13 | ¹H-NMR (CDCl₃, δ ppm): | 1.27 (3H, d), 1.99 (3H, s), 2.14 (3H, s), 2.55-2.71 (2H, m), 4.28-4.44 (1H, m), 5.67 (2H, s), 6.16 (1H, d), 6.56 (1H, d), 7.01 (1H, dd), 7.45 (1H, dd), 7.55 (1H, d), 7.70-7.81 |
| | | (2H, m), 8.04 (1H, s), 8.36 (1H, bs), 8.86 (1H, s) |
| 4-14 | ¹H-NMR | 1.28 (3H, d), 1.98 (3H, s), 2.00 (3H, s), 2.19 (3H, s), 2.59-2.72 (2H, m), 3.70 (2H, s), 4.30-4.43 (1H, m), 5.51 (2H, s), 6.20 (1H, d), 6.59 (1H, d), |
| | (CDCl₃, δ ppm): | 7.19 (1H, dd), 7.36-7.59 (6H, m), 7.78 (1H, d), 7.85 (1H, d), 8.46 (1H, s) |
| 4-15 | ¹H-NMR | 1.29 (3H, d), 2.00 (3H, s), 2.27 (3H, s), 2.57-2.74 (2H, m), 4.31-4.44 (1H, m), 5.59 (2H, s), 6.28 (1H, d), 6.45 (1H, d), 6.92 (1H, d), 7.06 (1H, d), |
| | (CDCl₃, δ ppm): | 7.17 (1H, dd), 7.45 (1H, dd), 7.55 (1H, d), 7.76 (1H, d), 7.83 (1H, d), 8.57 (1H, bs) |
| 4-19 | ¹H-NMR | 1.25 (3H, d), 1.99 (3H, s), 2.28 (3H, s), 2.52 (3H, s), 2.58 (1H, dd), 2.67 (1H, dd), 4.31-4.42 (1H, m), 5.57 (2H, s), 6.16 (1H, d), 6.45 (1H, d), |
| | (CDCl₃, δ ppm): | 6.97 (1H, s), 7.17 (1H, dd), 7.41-7.51 (2H, m), 7.64 (1H, d), 7.93 (1H, d), 8.33 (1H, bs) |
| 4-20 | ¹H-NMR | 1.23 (3H, d), 1.99 (3H, s), 2.20 (3H, s), 2.49-2.68 (2H, m), 2.50 (3H, s), 4.27-4.36 (1H, m), 5.31 (2H, s), 6.23 (1H, d), 6.50 (1H, d), 7.14-7.25 (2H, |
| | (CDCl₃, δ ppm): | m), 7.35-7.56 (5H, m), 8.76 (1H, s) |
| 4-24 | ¹H-NMR | 1.36 (3H, d), 2.13 (3H, s), 2.75 (2H, m), 3.92 (3H, s), 4.43 (1H, m), 5.59 (2H, s), 6.06 (1H, d), 6.96 (1H, d), 7.57 (4H, m), 8.08 (1H, d), |
| | (CDCl₃, δ ppm): | 8.87 (1H, s), 11.74 (1H, s). |
| 5-3 | ¹H-NMR | 1.3 (3H, d), 2.1 (3H, s), 2.6-2.9 (2H, m), 4.4 (1H, m), 5.4 (2H, s), 6.3 (1H, d), 7.0 (1H, s), 7.3-7.9 (5H, m), 8.6 (1H, s) |
| | (CDCl₃, δ ppm): | |
| 5-4 | ¹H-NMR | 1.3 (3H, d), 2.1 (3H, s), 2.6-2.9 (2H, m), 4.3-4.4 (1H, m), 5.4 (2H, s), 6.3 (1H, d), 7.0 (1H, s), 7.4-7.9 (5H, m), 8.6 (1H, s) |
| | (CDCl₃, δ ppm): | |
| 5-5 | ¹H-NMR | 1.37 (3H, d), 2.13 (3H, s), 2.67-2.85 (2H, m), 4.40-4.42 (1H, m), 5.29 (2H, s), 6.28 (1H, d), 7.44-7.47 (1H, m), 7.56-7.57 (1H, m), 7.75-7.80 (3H, |
| | (CDCl₃, δ ppm): | m), 7.92-7.95 (1H, m), 8.70 (1H, s) |
| 5-6 | ¹H-NMR | 1.34 (3H, d), 2.12 (3H, s), 2.62-2.85 (2H, m), 4.37-4.46 (1H, m), 5.33 (2H, s), 6.20 (1H, d), 7.07 (1H, d), 7.46-7.49 (1H, m), 7.57-7.62 (3H, m), |
| | (CDCl₃, δ ppm): | 7.77 (1H, s), 7.86 (1H, d), 8.37 (1H, s) |
| 5-10 | ¹H-NMR | 1.34 (3H, d), 2.09 (3H, s), 2.25 (6H, s), 2.60-2.84 (2H, m), 4.34-4.43 (1H, m), 5.44 (2H, s), 6.17 (1H, d), 6.92-8.05 (7H, m) |
| | (CDCl₃, δ ppm): | |
| 5-14 | ¹H-NMR | 1.3 (3H, d), 2.1 (3H, s), 2.6-2.9 (2H, m), 4.3-4.4 (1H, m), 5.5 (2H, s), 6.3 (1H, d), 7.4-7.8 (5H, m), 8.7 (1H, s) |
| | (CDCl₃, δ ppm): | |
| 5-15 | ¹H-NMR | 1.4 (3H, d), 2.1 (3H, s), 2.2-2.9 (2H, m), 4.4 (1H, m), 5.5 (2H, s), 6.3 (1H, d), 7.4-7.9 (5H, m), 8.7 (1H, s) |
| | (CDCl₃, δ ppm): | |
| 5-16 | ¹H-NMR | 1.4 (3H, d), 2.1 (3H, s), 2.6-2.9 (2H, m), 4.4 (1H, m), 5.4 (2H, s), 6.2 (1H, d), 7.0 (1H, s), 7.2-8.0 (5H, m), 8.4 (1H, s) |
| | (CDCl₃, δ ppm): | |
| 5-17 | ¹H-NMR | 1.4 (3H, d), 2.1 (3H, s), 2.6-2.9 (2H, m), 4.3-4.4 (1H, m), 5.4 (2H, s), 6.1 (1H, d), 7.0 (1H, s), 7.2-8.0 (5H, m), 8.4 (1H, s) |
| | (CDCl₃, δ ppm): | |
| 5-18 | ¹H-NMR | 1.4 (3H, d), 2.1 (3H, s), 2.7-2.9 (2H, m), 4.3-4.4 (1H, m), 5.5 (2H, s), 6.2 (1H, d), 7.2-8.0 (5H, m), 8.5 (1H, s) |
| | (CDCl₃, δ ppm): | |
| 5-21 | ¹H-NMR (CDCl₃, δ ppm): | 1.32 (3H, d), 2.09 (3H, s), 2.28 (3H, s), 2.61 (1H, dd), 2.80 (1H, dd), 4.30-4.42 (1H, m), 5.39 (2H, s), 6.22 (1H, d), 6.93 (1H, s), 7.08 (1H, s), 7.20 (1H, s), 7.42 (1H, t), 7.51 |
| | | (1H, d), 7.77 (1H, d), 8.28 (1H, bs) |
| 5-22 | ¹H-NMR | 1.31 (3H, d), 2.09 (3H, s), 2.28 (3H, s), 2.60 (1H, dd), 2.78 (1H, dd), 4.27-4.42 (1H, m), 5.46 (2H, s), 6.34 (1H, d), 7.12 (1H, s), 7.27 (1H, s), |
| | (CDCl₃, δ ppm): | 7.41 (1H, t), 7.49 (1H, d), 7.75 (1H, d), 8.45 (1H, bs) |
| 5-23 | ¹H-NMR | 1.4 (3H, d), 2.1 (3H, s), 2.7-2.9 (2H, m), 4.4 (1H, m), 5.5 (2H, s), 6.2 (1H, d), 7.2-8.0 (5H, m), 8.5 (1H, s) |
| | (CDCl₃, δ ppm): | |
| 5-24 | ¹H-NMR | 1.33 (3H, d), 2.09 (3H, s), 2.32 (3H, s), 2.50 (3H, s), 2.58-2.86 (2H, m), 4.34-4.39 (1H, m), 5.46 (2H, s), 6.29 (1H, d), 7.14 (1H, s), 7.28 (1H, s), |
| | (CDCl₃, δ ppm): | 7.40-7.47 (2H, m), 7.63-7.66 (1H, m), 8.30 (1H, s) |
| 6-14 | ¹H-NMR | 1.25 (3H, d), 1.95 (3H, s), 2.23 (3H, d), 2.52-2.65 (2H, m), 4.26-4.39 (1H, m), 5.57 (2H, s), 6.34 (1H, d), 7.05-7.12 (1H, m), 7.35-7.53 (2H, m), |
| | (CDCl₃, δ ppm): | 7.69 (1H, d), 7.88 (1H, d), 8.68 (1H, s) |
| 6-15 | ¹H-NMR | 1.33-1.47 (3H, d), 2.22 (3H, m), 2.31 (3H, m), 2.80-2.83 (2H, m), 4.39-4.59 (1H, m), 5.56 (2H, s), 7.13 (1H, t), 7.28-7.31 (1H, m), 7.40-7.45 (1H, |
| | (CDCl₃, δ ppm): | m), 7.51-7.54 (1H, m), 7.65 (1H, m), 7.85 (1H, d), 7.59 (1H, d), 8.59 (1H, m) |
| 6-16 | ¹H-NMR | 1.49 (3H, d), 2.23 (3H, s), 2.82 (3H, s), 3.26 (1H, dd), 3.35 (1H, dd), 4.57-4.66 (1H, m), 5.57 (2H, s), 6.63 (1H, s), 7.12 (1H, t), 7.46 (1H, t), |
| | (CDCl₃, δ ppm): | 7.56 (1H, d), 7.66 (1H, d), 7.83 (1H, d), 8.20 (1H, s) |
| 6-20 | ¹H-NMR | 1.26 (3H, d), 1.94 (3H, s), 2.16 (3H, d), 2.52-2.65 (2H, m), 4.28-4.37 (1H, m), 5.57 (2H, s), 6.25 (1H, d), 7.10 (1H, t), 7.37 (1H, t), 7.69-7.77 (2H, |
| | (CDCl₃, δ ppm): | m), 7.93 (1H, d), 8.60 (1H, s) |
| 6-26 | ¹H-NMR | 1.27 (3H, d), 1.94 (3H, s), 2.24 (3H, d), 2.52-2.65 (2H, m), 4.27-4.36 (1H, m), 5.57 (2H, s), 6.18 (1H, d), 7.11 (1H, t), 7.21 (1H, t), 7.78 (1H, d), |
| | (CDCl₃, δ ppm): | 7.96-8.01 (2H, m), 8.49 (1H, s) |
| 6-30 | ¹H-NMR | 1.21 (3H, d), 1.95 (3H, s), 2.23 (3H, d), 2.49-2.65 (5H, m), 4.28-4.37 (1H, m), 5.57 (2H, s), 6.28 (1H, d), 7.11 (1H, t), 7.41-7.48 (3H, m), 7.98 (1H, |
| | (CDCl₃, δ ppm): | d), 8.51 (1H, s) |
| 6-34 | ¹H-NMR | 1.25 (3H, d), 1.92 (3H, s), 2.24 (3H, d), 2.43-2.65 (2H, m), 3.28 (3H, s), 4.21-4.26 (1H, m), 5.58 (2H, s), 6.52 (1H, d), 7.16 (1H, t), 7.70 (1H, t), |
| | (CDCl₃, δ ppm): | 7.99-8.18 (4H, m) |
| 6-35 | ¹H-NMR | 1.19 (3H, d), 2.17 (3H, s), 2.17-2.88 (5H, m), 4.26-4.57 (1H, m), 5.58 (2H, s), 7.08-7.20 (1H, m), 7.59-8.11 (5H, m), 8.48-8.59 (1H, m) |
| | (CDCl₃, δ ppm): | |
| 6-36 | ¹H-NMR | 1.35 (3H, d), 2.23 (3H, s), 2.83 (3H, s), 3.17 (1H, dd), 3.26 (3H, s), 3.35 (1H, dd), 4.44-4.58 (1H, m), 5.57 (2H, s), 7.07-7.20 (2H, m), 7.68 (1H, |
| | (CDCl₃, δ ppm): | dd), 7.83-8.15 (4H, m) |
| 6-65 | ¹H-NMR | 1.27 (3H, d), 1.99 (3H, s), 2.56-2.68 (2H, m), 4.30-4.43 (1H, m), 5.60 (2H, s), 6.12 (1H, d), 7.21-7.27 (1H, m), 7.47 (1H, t), 7.58 (1H, d), 7.69 (1H, |
| | (CDCl₃, δ ppm): | d), 8.35 (1H, d), 8.81 (1H, s) |
| 7-4 | ¹H-NMR | 1.18 (3H, d), 1.93 (3H, s), 2.22 (3H, s), 2.51 (1H, dd), 2.60 (1H, dd), 4.11-4.21 (1H, m), 5.50 (2H, s), 6.92 (1H, d), 7.05 (1H, d), 7.13 (1H, s), |
| | (CD₃CN, δ ppm): | 7.48 (1H, dd), 7.59 (1H, d), 7.69 (1H, d), 7.85 (1H, d), 8.46 (1H, bs) |
| 7-5 | ¹H-NMR | 1.22 (3H, d), 2.21 (3H, s), 2.43 (3H, s), 2.60-3.00 (2H, m), 4.22-4.33 (1H, m), 5.63 (2H, s), 7.04 (1H, d), 7.48-7.67 (5H, m), 8.40-8.58 (1H, m), |
| | (DMSO-d₆, δ ppm): | 9.50 (1H, bs) |
| 7-6 | ¹H-NMR (CD₃CN, δ ppm): | 1.35 (3H, d), 2.24 (3H, s), 2.81 (3H, s), 3.08 (1H, dd), 3.45 (1H, dd), 4.45-4.55 (1H, m), 5.51 (2H, s), 7.00-7.10 (2H, m), 7.13 (1H, s), 7.50 (1H, dd), 7.60 (1H, d), 7.68 (1H, d), |
| | | 7.73 (1H, d), 8.34 (1H, bs) |
| 7-7 | ¹H-NMR | 1.24 (3H, d), 1.94 (3H, s), 2.30 (3H, s), 2.57-2.59 (2H, m), 4.29-4.38 (1H, m), 5.54 (2H, s), 6.14 (1H, d), 7.06 (1H, d), 7.47 (1H, t), 7.57 (1H, d), |
| | (CDCl₃, δ ppm): | 7.71 (1H, d), 8.21 (1H, d), 8.68 (1H, s) |
| 7-11 | ¹H-NMR | 1.22 (3H, d), 2.22 (3H, s), 2.42 (3H, s), 2.67-2.96 (2H, m), 4.22-4.33 (1H, m), 5.57 (2H, s), 7.11 (1H, d), 7.43 (1H, dd), 7.51 (1H, s), 7.55 (1H, d), |
| | (DMSO-d₆, δ ppm): | 7.66-7.72 (1H, m), 7.78 (1H, d), 8.39-8.54 (1H, m), 9.47 (1H, bs) |
| 7-12 | ¹H-NMR | 1.35 (3H, d), 2.23 (3H, s), 2.81 (3H, s), 3.08 (1H, dd), 3.35 (1H, dd), 4.45-4.53 (1H, m), 5.52 (2H, s), 7.02 (1H, d), 7.07 (1H, d), 7.14 (1H, s), |
| | (CD₃CN, δ ppm): | 7.42 (1H, dd), 7.70-7.80 (3H, d), 8.32 (1H, bs) |
| 7-16 | ¹H-NMR | 1.20 (3H, d), 1.90 (3H, s), 2.23 (3H, s), 2.49 (1H, dd), 2.60 (1H, dd), 4.10-4.18 (1H, m), 5.50 (2H, s), 6.82 (1H, d), 7.06 (1H, d), 7.13 (1H, s), |
| | (CD₃CN, δ ppm): | 7.24 (1H, dd), 7.74 (1H, d), 7.90 (1H, d), 8.02 (1H, d), 8.39 (1H, bs) |
| 7-17 | ¹H-NMR | 1.23 (3H, d), 2.21 (3H, s), 2.40 (3H, s), 2.65-3.00 (2H, m), 4.20-4.32 (1H, m), 5.57 (2H, s), 7.11 (1H, d), 7.25 (1H, dd), 7.50 (1H, s), 7.57 (1H, d), |
| | (DMSO-d₆, δ ppm): | 7.66-7.72 (1H, m), 8.01 (1H, d), 8.35-8.51 (1H, m), 9.40 (1H, bs) |
| 7-18 | ¹H-NMR | 1.37 (3H, d), 2.22 (3H, s), 2.81 (3H, s), 3.09 (1H, dd), 3.37 (1H, dd), 4.41-4.51 (1H, m), 5.51 (2H, s), 7.03 (1H, d), 7.08 (1H, d), 7.14 (1H, s), |
| | (CD₃CN, δ ppm): | 7.25 (1H, dd), 7.72-7.78 (2H, m), 8.03 (1H, d), 8.32 (1H, bs) |
| 7-22 | ¹H-NMR | 1.15 (3H, d), 2.03 (3H, s), 2.25 (3H, s), 2.47-2.64 (2H, m), 2.50 (3H, s), 4.00-4.07 (1H, m), 5.59 (2H, s), 7.10 (1H, d), 7.38-7.57 (4H, m), 7.63 (1H, |
| | (DMSO-d₆, δ ppm): | s), 8.21 (1H, d), 9.80 (1H, bs) |
| 7-44 | ¹H-NMR | 1.28 (3H, d), 2.01 (3H, s), 2.57-2.69 (2H, m), 4.31-4.44 (1H, m), 5.54 (2H, s), 6.10 (1H, d), 7.35 (1H, d), 7.47 (1H, t), 7.59 (1H, d), 7.68 (1H, d), |
| | (CDCl₃, δ ppm): | 8.46 (1H, d), 8.80 (1H, s) |
| 8-2 | ¹H-NMR | 1.15 (6H, d), 4.09-4.16 (1H, m), 5.32 (2H, s), 6.46 (1H, d), 7.19-7.22 (2H, m), 7.26-7.39 (3H, m), 7.61-7.64 (2H, m), 7.73 (2H, d), 9.99 (1H, s) |
| | (CDCl₃, δ ppm): | |
| 8-3 | ¹H-NMR | 1.26 (3H, d), 2.06 (3H, s), 2.64 (2H, d), 4.31-4.34 (1H, m), 5.34 (2H, s), 6.36 (1H, d), 7.25-7.27 (2H, m), 7.47-7.54 (3H, m), 7.63 (1H, s), |
| | (CDCl₃, δ ppm): | 7.72-7.74 (2H, m), 7.81-7.83 (1H, m), 9.62 (1H, s) |
| 8-4 | ¹H-NMR | 1.33-1.42 (3H, m), 2.38-2.44 (3H, m), 2.84-2.90 (2H, m), 4.45-4.48 (1H, m), 5.34 (2H, s), 7.25-7.31 (3H, m), 7.45-7.51 (3H, m), 7.68-7.72 (4H, |
| | (CDCl₃, δ ppm): | m), 9.53-9.64 (1H, m) |
| 8-6 | ¹H-NMR | 1.16 (6H, d), 4.22-4.25 (1H, m), 5.32 (2H, s), 5.93 (1H, d), 7.14-7.17 (2H, m), 7.43-7.46 (1H, m), 7.53-7.56 (1H, m), 7.61 (1H, s), 7.74 (1H, d), |
| | (CDCl₃, δ ppm): | 8.10 (1H), 8.50 (1H, s) |
| 8-7 | ¹H-NMR | 1.24 (3H, d), 1.96 (3H, s), 2.51-2.69 (1H, m), 4.26-4.38 (1H, m), 5.42 (2H, s), 6.23 (1H, d), 6.91-7.65 (7H, m), 9.07 (1H, br s) |
| | (CDCl₃, δ ppm): | |
| 8-8 | ¹H-NMR | 1.25 (3H, d), 1.97 (3H, s), 2.58-2.64 (2H, m), 4.35-4.36 (1H, m), 5.33 (2H, s), 6.18 (1H, d, J = 8.2 Hz), 7.24-7.29 (2H, m), 7.41-7.52 (2H, m), |
| | (CDCl₃, δ ppm): | 7.62-7.73 (4H, m), 9.02 (1H, s) |
| 8-13 | ¹H-NMR | 1.22 (3H, d), 1.96 (3H, s), 2.45-2.72 (2H, m), 4.19-4.28 (1H, m), 5.46 (2H, s), 6.69 (1H, d), 7.18-7.24 (5H, m), 7.40-7.44 (1H, m), 7.57-7.60 (2H, |
| | (CDCl₃, δ ppm): | m), 9.63 (1H, s) |
| 8-14 | ¹H-NMR (CDCl₃, δ ppm): | 1.23 (3H, d), 1.96 (3H, s), 2.50 (1H, dd), 2.65 (1H, dd), 4.18-4.32 (1H, m), 5.48 (2H, s), 6.64 (1H, d), 7.17 (2H, d), 7.22-7.31 (2H, m), 7.46 (1H, d), 7.61 (2H, d), 9.58 (1H, bs) |
| 8-15 | ¹H-NMR | 1.24 (3H, d), 1.96 (3H, s), 2.55 (1H, dd), 2.64 (1H, dd), 4.28-4.40 (1H, m), 5.53 (2H, s), 6.24 (1H, d), 7.23 (1H, s), 7.37-7.50 (3H, m), |
| | (CDCl₃, δ ppm): | 7.60-7.69 (3H, m), 9.09 (1H, bs) |
| 8-16 | ¹H-NMR | 1.24 (3H, d), 1.95 (3H, s), 2.53 (1H, dd), 2.65 (1H, dd), 4.27-4.39 (1H, m), 5.42 (2H, s), 6.15 (1H, d), 6.91 (1H, s), 7.15-7.22 (2H, m), 7.33 (1H, |
| | (CDCl₃, δ ppm): | t), 7.55-7.74 (4H, m), 8.94 (1H, bs) |
| 8-18 | ¹H-NMR | 1.27 (3H, d), 1.96 (3H, s), 2.54 (1H, dd), 2.67 (1H, dd), 4.24-4.38 (1H, m), 5.49 (2H, s), 6.38 (1H, d), 7.17-7.34 (3H, m), 7.53-7.67 (4H, m), |
| | (CDCl₃, δ ppm): | 9.33 (1H, bs) |
| 9-2 | ¹H-NMR | 1.23 (3H, d), 1.92 (3H, s), 2.30 (3H, s), 2.55 (1H, dd), 2.60 (1H, dd), 4.27-4.39 (1H, m), 5.41 (2H, s), 6.14 (1H, d), 6.91 (1H, s), 7.10 (1H, s), |
| | (CDCl₃, δ ppm): | 7.12 (1H, d), 7.61 (1H, d), 7.71 (1H, d), 8.09 (1H, d), 8.34 (1H, bs) |
| 9-5 | ¹H-NMR | 1.23 (3H, d), 1.92 (3H, s), 2.31 (3H, s), 2.54 (1H, dd), 2.59 (1H, dd), 4.25-4.38 (1H, m), 5.47 (2H, s), 6.35 (1H, d), 7.16 (1H, s), 7.17 (1H, d), |
| | (CDCl₃, δ ppm): | 7.58 (1H, d), 7.66 (1H, d), 8.10 (1H, d), 8.45 (1H, bs) |
| 9-8 | ¹H-NMR | 1.22 (3H, d), 1.91 (3H, s), 2.30 (3H, s), 2.54 (1H, dd), 2.59 (1H, dd), 4.25-4.38 (1H, m), 5.49 (2H, s), 6.35 (1H, d), 7.15 (1H, s), 7.17 (1H, d), |
| | (CDCl₃, δ ppm): | 7.54-7.70 (2H, m), 8.10 (1H, d), 8.46 (1H, bs) |
| 10-13 | ¹H-NMR | 1.19 (3H, d), 1.90 (3H, s), 2.27 (3H, s), 2.49 (1H, dd), 2.61 (1H, dd), 4.09-4.19 (1H, m), 5.55 (2H, s), 6.88 (1H, d), 7.16 (1H, d), 7.19 (1H, s), |
| | (DMSO-d₆, δ ppm): | 7.40 (1H, d), 7.44 (1H, d), 7.79 (1H, d), 8.40 (1H, bs) |
| 11-25 | ¹H-NMR | 1.29 (3H, d), 2.28 (3H, s), 2.47 (3H, s), 2.66-2.87 (2H, m), 4.33-4.51 (1H, m), 5.54 (2H, s), 6.96 (1H, d), 7.16-7.23 (2H, m), 7.47-7.53 (2H, m), |
| | (CD₃CN, δ ppm): | 7.73 (1H, d), 8.23 (1H, bs) |
| 12-6 | ¹H-NMR | 1.19 (3H, d), 2.05 (3H, s), 2.17 (3H, s), 2.51-2.64 (2H, m), 4.14-4.26 (1H, m), 5.48 (2H, s), 6.56-6.61 (1H, m), 7.18-7.22 (2H, m), 7.47-7.51 (2H, |
| | (CDCl₃, δ ppm): | m), 7.77 (1H, s), 7.95-7.98 (1H, d) |
| 13-3 | ¹H-NMR | 1.27 (3H, d), 2.04 (3H, s), 2.34 (3H, s), 2.60-2.65 (2H, m), 4.31-4.36 (1H, m), 5.32 (2H, s), 6.34 (1H, d), 7.15-7.19 (2H, m), 7.57-7.61 (4H, m), |
| | (CDCl₃, δ ppm): | 7.86 (1H, d), 8.12 (1H, d), 8.85 (1H, s) |
| 13-5 | ¹H-NMR | 1.33-1.42 (3H, m), 2.38-2.44 (3H, m), 2.84-2.90 (2H, m), 4.45-4.48 (1H, m), 5.34 (2H, s), 7.25-7.31 (3H, m), 7.45-7.51 (3H, m), 7.68-7.72 (4H, |
| | (CDCl₃, δ ppm): | m), 9.53-9.64 (1H, m) |
| 13-8 | ¹H-NMR | 1.16 (3H, d), 1.28 (3H, t), 1.97 (3H, s), 2.29 (3H, s), 2.45-2.60 (2H, m), 2.75 (2H, q), 4.23-4.36 (1H, m), 5.40 (2H, s), 6.09 (1H, d), 6.91 (1H, s), |
| | (CDCl₃, δ ppm): | 7.07-7.15 (2H, m), 7.38-7.48 (2H, m), 7.60 (1H, d), 8.08 (1H, bs), 8.19 (1H, d) |
| 13-11 | ¹H-NMR | 1.23 (3H, d), 1.43 (3H, t), 1.97 (3H, s), 2.30 (3H, s), 2.50-2.66 (2H, m), 4.10 (2H, q), 4.28-4.40 (1H, m), 5.40 (2H, s), 6.26 (1H, d), 6.90 (1H, s), |
| | (CDCl₃, δ ppm): | 7.00-7.16 (3H, m), 7.35-7.47 (2H, m), 8.09 (1H, d), 8.42 (1H, bs) |
| 3-17 | ¹H-NMR | 2.33 (3H, s), 2.99 (3H, d), 5.32 (2H, s), 6.06 (1H, d), 7.15-7.17 (1H, m), 7.44-7.47 (1H, m), 7.55-7.56 (1H, m), 7.64 (1H, s), 7.74 (1H, d), |
| | (CDCl₃, δ ppm): | 8.05 (1H, d), 8.41 (1H, s) |
| 13-18 | ¹H-NMR | 0.87 (3H, t), 1.10 (3H, d), 1.52 (2H, quintet), 2.30 (3H, s), 3.37 (2H, q), 5.41 (2H, s), 5.99-6.07 (1H, m), 6.91 (1H, s), 7.09-7.15 (2H, m), |
| | (CDCl₃, δ ppm): | 7.44 (1H, dd), 7.55 (1H, d), 7.74 (1H, d), 8.03 (1H, d), 8.37 (1H, bs) |
| 13-19 | ¹H-NMR (CDCl₃, δ ppm): | 1.16 (6H, d), 2.34 (3H, s), 4.22-4.26 (1H, m), 5.31 (2H, s), 5.85 (1H, d), 7.15-7.17 (2H, m), 7.44-7.47 (1H, m), 7.54-7.57 (1H, m), 7.62 (1H, s), 7.76 (1H, d), 8.12 (1H, d), |
| | | 8.48 (1H, s) |
| 13-20 | ¹H-NMR | 0.87 (3H, t), 1.10 (3H, d), 1.40-1.52 (2H, m), 2.30 (3H, s), 3.97-4.12 (1H, m), 5.41 (2H, s), 5.88 (1H, d), 6.91 (1H, s), 7.07-7.13 (2H, m), |
| | (CDCl₃, δ ppm): | 7.43 (1H, dd), 7.54 (1H, d), 7.72 (1H, d), 8.06 (1H, d), 8.43 (1H, bs) |
| 13-21 | ¹H-NMR | 0.68 (3H, t), 1.11 (3H, d), 1.15-1.42 (4H, m), 2.30 (3H, s), 4.06-4.19 (1H, m), 5.41 (2H, s), 5.97 (1H, d), 6.90 (1H, s), 7.06-7.14 (2H, m), |
| | (CDCl₃, δ ppm): | 7.40 (1H, dd), 7.51 (1H, d), 7.69 (1H, d), 8.07 (1H, d), 8.48 (1H, bs) |
| 13-22 | ¹H-NMR | 0.83 (3H, t), 1.08 (3H, d), 1.39-1.50 (2H, m), 2.29 (3H, s), 3.90-4.03 (1H, m), 5.42 (2H, s), 6.22 (1H, d), 6.91 (1H, s), 7.10-7.17 (2H, m), |
| | (CDCl₃, δ ppm): | 7.48-7.58 (3H, m), 7.98 (1H, d) |
| 13-23 | ¹H-NMR | 1.24 (3H, d), 1.91 (3H, s), 2.37 (3H, s), 2.53-2.61 (2H, m), 4.26-4.40 (1H, m), 6.30 (1H, d), 6.45 (1H, s), 6.98 (1H, s), 7.29-7.60 (4H, m), |
| | (CDCl₃, δ ppm): | 7.72 (1H, d), 8.24 (1H, d), 8.59 (1H, bs) |
| 13-24 | ¹H-NMR | 1.24 (3H, d), 1.95 (3H, s), 2.30 (3H, s), 2.51-2.65 (2H, m), 4.29-4.38 (1H, m), 5.39 (2H, s), 6.07-6.29 (1H, br), 7.05-8.35 (7H, m) |
| | (CDCl₃, δ ppm): | |
| 13-25 | ¹H-NMR | 1.27 (3H, d), 2.01 (3H, s), 2.42 (3H, s), 2.59-2.66 (2H, m), 4.36-4.39 (1H, m), 6.29 (1H, d), 7.47-7.54 (3H, m), 7.64-7.65 (1H, m), 7.75-7.78 (2H, |
| | (CDCl₃, δ ppm): | m), 7.93-7.94 (2H, m), 8.04 (1H, s), 8.22-8.24 (2H, m), 8.56 (1H, s) |
| 13-26 | ¹H-NMR | 1.31 (3H, d), 1.97-2.04 (3H, m), 2.14 (3H, s), 2.54-2.85 (2H, m), 4.35-4.38 (1H, m), 5.30 (2H, s), 6.08 (1H, d), 7.14-8.05 (12H, m) |
| | (CDCl₃, δ ppm): | |
| 13-29 | ¹H-NMR | 1.25 (3H, d), 1.96 (3H, s), 2.26 (3H, s), 2.55-2.60 (2H, m), 4.30-4.33 (1H, m), 5.22 (2H, s), 5.51 (2H, s), 6.14 (1H, d), 6.51 (1H, d), 6.88-6.91 (1H, |
| | (CDCl₃, δ ppm): | m), 7.02-7.06 (2H, m), 7.28-7.52 (3H, m), 7.67 (1H, d), 7.97 (1H, d), 8.42 (1H, s) |
| 13-30 | ¹H-NMR | 0.86 (3H, t), 1.24 (3H, d), 1.92 (3H, d), 2.18-2.21 (1H, m), 2.30 (3H, s), 2.53-2.59 (4H, m), 4.34-4.35 (1H, m), 5.21-5.24 (1H, m), 6.11 (1H, d), |
| | (CDCl₃, δ ppm): | 6.85 (1H, s), 7.24-7.56 (5H, m), 7.74 (1H, d), 8.04 (1H, d), 8.32 (1H, s) |
| 13-31 | ¹H-NMR | 1.23 (3H, d), 1.31 (3H, t), 1.93 (3H, s), 2.28 (3H, s), 2.50-2.61 (2H, m), 4.26-4.35 (3H, m), 5.28 (2H, s), 6.92 (1H, d), 7.10-7.13 (2H, m), |
| | (CDCl₃, δ ppm): | 7.30-7.37 (1H, m), 7.43-7.46 (1H, m), 7.58 (1H, d), 7.71 (1H, s), 7.96 (1H, d), 8.72 (1H, s) |
| 13-33 | ¹H-NMR | 1.23 (3H, t), 1.24 (3H, d), 1.90 (3H, s), 2.37 (3H, s), 2.50-2.64 (2H, m), 4.19-4.40 (3H, m), 6.10 (1H, s), 6.21 (1H, d), 6.92 (1H, s), 7.28-7.36 (2H, |
| | (CDCl₃, δ ppm): | m), 7.44 (1H, dd), 7.54 (1H, d), 7.73 (1H, d), 8.18 (1H, d), 8.47 (1H, bs) |
| 13-34 | ¹H-NMR | 1.2 (3H, d), 2.0 (3H, s), 2.5-2.7 (2H, m), 4.3-4.4 (1H, m), 5.4 (2H, s), 6.3 (1H, d), 6.9 (1H, s), 7.2-8.4 (6H, m), 8.6 (1H, s) |
| | (CDCl₃, δ ppm): | |
| 13-35 | ¹H-NMR | 1.25 (3H, d), 1.95 (3H, s), 2.33 (3H, s), 2.54-2.62 (2H, m), 4.32-4.35 (1H, m), 5.31 (2H, s), 6.23 (1H, d), 7.18-7.21 (2H, m), 7.45-7.53 (5H, m), |
| | (CDCl₃, δ ppm): | 7.61-7.64 (2H, m), 7.73 (1H, d), 8.11 (1H, d), 8.44 (1H, s) |
| 13-36 | ¹H-NMR | 1.25 (3H, d), 1.96 (3H, s), 2.34 (3H, s), 2.55-2.63 (2H, m), 4.29-4.38 (1H, m), 5.50 (2H, s), 6.35 (1H, d), 7.18-7.20 (2H, m), 7.41-7.44 (1H, m), |
| | (CDCl₃, δ ppm): | 7.51-7.54 (1H, m), 7.69-7.78 (3H, m), 7.95 (1H, s), 8.07 (1H, d), 8.48 (1H, s) |
| 13-37 | ¹H-NMR | 1.25 (3H, d), 1.98 (3H, s), 2.26 (3H, s), 2.31-2.66 (2H, m), 4.23-4.41 (1H, m), 5.29 (2H, s), 6.14 (1H, d), 6.64-8.33 (12H, m) |
| | (CDCl₃, δ ppm): | |
| 13-38 | ¹H-NMR (CDCl₃, δ ppm): | 1.24 (3H, d), 1.89 (3H, d), 1.94 (3H, s), 2.28 (3H, s), 2.48-2.66 (2H, m), 2.51 (3H, s), 4.26-4.39 (1H, m), 5.54 (1H, q), 6.40 (1H, d), 6.53 (1H, bs), 7.03-7.12 (2H, m), 7.36-7.52 |
| | | (3H, m), 7.67 (1H, d), 7.98 (1H, d), 8.42 (1H, bs) |
| 13-40 | ¹H-NMR | 1.25 (3H, d), 1.89 (3H, d), 1.95 (3H, s), 2.29 (3H, s), 2.50-2.66 (2H, m), 2.51 (3H, s), 4.26-4.41 (1H, m), 5.54 (1H, q), 6.16 (1H, d), 6.53 (1H, bs), |
| | (CDCl₃, δ ppm): | 7.02-7.14 (2H, m), 7.35-7.60 (3H, m), 7.74 (1H, d), 8.05 (1H, d), 8.37 (1H, bs) |
| 13-41 | ¹H-NMR | 1.24 (3H, d), 1.94 (3H, s), 2.30 (3H, s), 2.50-2.65 (2H, m), 4.29-4.38 (1H, m), 5.51 (2H, s), 6.16 (1H, d), 7.09-8.36 (7H, m) |
| | (CDCl₃, δ ppm): | |
| 13-44 | ¹H-NMR | 1.21 (3H, t), 1.22 (3H, d), 1.942 (3H, s), 2.25 (3H, s), 2.46-2.66 (4H, m), 4.24-4.38 (1H, m), 5.29 (2H, s), 6.36 (1H, s), 6.44-6.54 (1H, m), |
| | (CDCl₃, δ ppm): | 6.88-6.99 (2H, m), 7.33-7.98 (4H, m), 8.42 (1H, bs) |
| 13-45 | ¹H-NMR | 1.29 (3H, d), 2.02 (3H, s), 2.63-2.67 (2H, m), 4.37-4.39 (1H, m), 5.32 (2H, s), 6.13 (1H, d), 7.29-7.31 (1H, m), 7.45-7.47 (1H, m), 7.56-7.59 (1H, |
| | (CDCl₃, δ ppm): | m), 7.68-7.69 (2H, m), 7.79 (1H, d), 8.28 (1H, d), 8.40 (1H, s) |
| 13-46 | ¹H-NMR | 1.27 (3H, d), 2.00 (3H, s), 2.61-2.64 (2H, m), 4.36-4.39 (1H, m), 5.34 (2H, s), 6.18 (1H, d), 7.06-7.10 (2H, m), 7.44-7.46 (1H, m), 7.54-7.57 (1H, |
| | (CDCl₃, δ ppm): | m), 7.69-7.72 (2H, m), 8.41-8.44 (1H, m), 8.76 (1H, s) |
| 13-47 | ¹H-NMR | 1.24 (3H, d), 1.94 (3H, s), 2.29 (3H, s), 2.51-2.65 (2H, m), 4.29-4.38 (1H, m), 5.23 (2H, s), 6.16 (1H, d), 6.20 (1H, s), 6.46 (1H, t), 7.09-7.11 (2H, |
| | (CDCl₃, δ ppm): | m), 7.44 (1H, t), 7.55 (1H, d), 7.74 (1H, d), 8.06 (1H, d), 8.35 (1H, s) |
| 13-48 | ¹H-NMR | 1.2 (3H, d), 1.9 (3H, s), 2.3 (3H, s), 2.5-2.6 (2H, m), 4.3 (1H, m), 5.5 (2H, s), 6.2 (1H, d), 7.0 (1H, s), 7.1-8.1 (6H, m), 8.4 (1H, s) |
| | (CDCl₃, δ ppm): | |
| 13-49 | ¹H-NMR | 1.25 (3H, d), 1.95 (3H, s), 2.33 (3H, s), 2.53-2.61 (2H, m), 4.32-4.34 (1H, m), 5.34 (2H, s), 6.33-6.36 (1H, m), 7.19-7.20 (2H, m), 7.42-7.45 (1H, |
| | (CDCl₃, δ ppm): | m), 7.51-7.53 (2H, m), 7.70 (1H, d), 7.79 (2H, s), 7.84 (1H, s), 8.09 (1H, d), 8.49 (1H, s) |
| 13-51 | ¹H-NMR | 1.25 (3H, d), 1.96 (3H, s), 2.30 (3H, s), 2.48-2.68 (2H, m), 4.25-4.45 (1H, m), 5.31 (2H, s), 6.16 (1H, d), 6.62 (1H, s), 7.11-8.36 (12H, m) |
| | (CDCl₃, δ ppm): | |
| 13-52 | ¹H-NMR | 1.25 (3H, d), 1.97 (3H, s), 2.32 (3H, s), 2.51-2.66 (2H, m), 4.25-4.41 (1H, m), 5.28 (2H, s), 6.25 (1H, d), 7.15-8.41 (12H, m) |
| | (CDCl₃, δ ppm): | |
| 13-53 | ¹H-NMR | 1.25 (3H, d), 1.97 (3H, s), 2.34 (3H, s), 2.53-2.66 (2H, m), 4.21-4.25 (1H, m), 5.31 (2H, s), 6.17 (1H, d), 7.18-8.41 (12H, m) |
| | (CDCl₃, δ ppm): | |
| 13-54 | ¹H-NMR | 1.23 (3H, d), 1.93 (3H, s), 2.30 (3H, s), 2.48-2.66 (2H, m), 4.23-4.39 (1H, m), 5.42 (2h, s), 6.15 (1H, d), 7.11-8.33 (12H, m) |
| | (CDCl₃, δ ppm): | |
| 13-55 | ¹H-NMR | 1.24 (3H, d), 1.96 (3H, s), 2.30 (3H, s), 2.50-2.66 (2H, m), 4.23-4.41 (1H, m), 5.31 (2H, s), 6.25 (1H, d), 6.60 (1H, s), 7.11-8.38 (12H, m) |
| | (CDCl₃, δ ppm): | |
| 13-56 | ¹H-NMR | 1.25 (3H, d), 1.97 (3H, s), 2.32 (3H, s), 2.51-2.66 (2H, m), 4.25-4.41 (1H, m), 5.28 (2H, s), 6.25 (1H, d), 7.15-8.41 (12H, m) |
| | (CDCl₃, δ ppm): | |
| 13-57 | ¹H-NMR | 1.21 (3H, d), 1.96 (3H, s), 2.34 (3H, s), 2.48-2.68 (2H, m), 4.21-4.27 (1H, m), 5.31 (2H, s), 6.15 (1H, d), 7.18-8.41 (12H, m) |
| | (CDCl₃, δ ppm): | |
| 13-59 | ¹H-NMR (CDCl₃, δ ppm): | 0.86 (3H, t), 1.24-1.26 (3H, m), 2.09-2.16 (7H, m), 2.58-2.69 (4H, m), 4.31 (1H, s), 5.23-5.26 (1H, m), 6.87 (1H, s), 7.40-7.79 (8H, m), 8.68 (1H, s) |
| 13-60 | ¹H-NMR | 1.47 (3H, d), 2.31 (3H, s), 2.77 (3H, s), 3.11-3.34 (2H, m), 4.54-4.63 (1H, m), 5.31 (2H, s), 6.79 (1H, d), 7.17-7.20 (2H, m), 7.51 (9H, m), |
| | (CDCl₃, δ ppm): | 7.96 (1H, d), 8.16 (1H, s) |
| 13-64 | ¹H-NMR | 1.24-1.42 (3H, m), 2.16-2.30 (6H, m), 2.76-2.81 (2H, m), 4.40-4.43 (1H, m), 5.30 (2H, s), 7.11-7.14 (2H, m), 7.37-7.76 (5H, m), 7.99 (1H, d), |
| | (CDCl₃, δ ppm): | 8.57 (1H, s) |
| 13-66 | ¹H-NMR | 1.35 (3H, d), 2.30 (3H, s), 2.79 (3H, s), 2.81-2.88 (2H, m), 4.47-4.54 (1H, m), 5.44 (2H, s), 7.20-8.11 (8H, m), 8.89 (1H, br) |
| | (Acetone-d₆, δ ppm): | |
| 13-67 | ¹H-NMR | 1.2 (3H, d), 2.2 (3H, s), 2.4 (3H, s), 2.7-3.0 (2H, m), 4.2-4.3 (1H, m), 5.3 (2H, s), 6.6 (1H, s), 7.0-8.7 (8H, m), 9.7 (1H, s) |
| | (CDCl₃, δ ppm): | |
| 13-68 | ¹H-NMR | 1.3 (3H/2, d), 1.4 (3H/2, d), 2.2 (3H, s), 2.6 (3H/2, s), 2.6 (3H/2, s), 2.7-2.9 (2H, m), 4.4-4.5 (1H, m), 5.5 (2H, s), 7.0 (1H, s), 7.1-8.0 (7H, m), |
| | (CDCl₃, δ ppm): | 8.4 (1H, d) |
| 13-71 | ¹H-NMR | 1.35 and 1.45 (3H, d and d), 2.18-2.32 (6H, br m), 2.70-2.90 (2H, br m), 4.37-4.58 (1H, br m), 5.29 (2H, s), 6.61-8.29 (14H, m) |
| | (CDCl₃, δ ppm): | |
| 13-77 | ¹H-NMR | 1.26 (3H, d), 1.97 (3H, s), 2.34 (3H, s), 2.54-2.63 (2H, m), 4.32-4.37 (1H, m), 5.30 (2H, s), 6.20 (1H, d), 7.14-7.19 (2H, m), 7.46 (1H, dd), |
| | (CDCl₃, δ ppm): | 7.56 (1H, d), 7.74-7.79 (2H, m), 8.19 (1H, d), 8.48 (1H, bs) |
| 13-78 | ¹H-NMR | 1.26 (3H, d), 1.97 (3H, s), 2.34 (3H, s), 2.52-2.65 (2H, m), 4.30-4.39 (1H, m), 5.32 (2H, s), 6.17 (1H, d), 7.14-7.19 (2H, m), 7.47 (1H, dd), |
| | (CDCl₃, δ ppm): | 7.56 (1H, d), 7.76 (1H, d), 7.81 (1H, s), 8.20 (1H, d), 8.47 (1H, bs) |
| 13-80 | ¹H-NMR | 1.32 (3H, d), 2.28 (3H, s), 2.81 (3H, s), 3.0-3.4 (2H, m), 4.4-4.6 (1H, m), 5.35 (2H, s), 6.9-7.1 (1H, m), 7.1-7.3 (2H, m), 7.4-7.7 (4H, m), |
| | (CDCl₃, δ ppm): | 8.2-8.4 (2H, m) |
| 13-82 | ¹H-NMR | 1.24 (3H, d), 1.98 (3H, s), 2.33 (3H, s), 2.48-2.65 (5H, m), 4.31-4.36 (1H, m), 5.32 (2H, s), 6.15 (1H, d), 7.13-7.19 (2H, m), 7.43-7.51 (2H, m), |
| | (CDCl₃, δ ppm): | 7.62 (1H, d), 7.80 (1H, s), 8.26 (1H, d), 8.37 (1H, s) |
| 13-84 | ¹H-NMR | 1.07 (3H, d), 1.92 (3H, s), 2.19-2.64 (5H, m), 3.88-4.00 (1H, m), 5.49 (2H, s), 7.17-7.23 (2H, m), 7.65 (1H, d), 7.79 (1H, dd), 7.97 (1H, d), |
| | (DMSO-d₆, δ ppm): | 8.08 (1H, d), 8.53 (1H, d), 9.03 (1H, s), 9.49 (1H, s) |
| 13-85 | ¹H-NMR | 1.41 (3H, d), 2.28 (3H, s), 2.72 (3H, s), 3.04-3.30 (2H, m), 4.53-4.55 (1H, m), 5.30 (2H, s), 7.06-7.64 (10H, m), 7.82 (1H, d), 8.36 (1H, s) |
| | (CDCl₃, δ ppm): | |
| 13-89 | ¹H-NMR | 1.45 (3H, d), 2.28 (3H, s), 2.74 (3H, s), 3.10-3.33 (2H, m), 4.54-4.58 (1H, m), 5.29 (2H, s), 6.91-6.94 (1H, m), 7.10-7.13 (2H, m), 7.45-7.54 (3H, |
| | (CDCl₃, δ ppm): | m), 7.73 (1H, s), 7.93-7.96 (1H, m), 8.25 (1H, s) |
| 13-90 | ¹H-NMR | 1.24 (3H, d), 2.29 (3H, s), 2.77 (3H, s), 3.10-3.42 (2H, m), 4.53-4.67 (1H, m), 5.52 (2H, s), 6.47 (1H, d), 7.11-7.93 (7H, m) |
| | (CDCl₃, δ ppm): | |
| 13-94 | ¹H-NMR | 1.26 (3H, dd), 1.94 (3H, s), 2.31 (3H, s), 2.61-2.52 (2H, m), 4.33-4.29 (1H, m), 5.24 (2H, s), 6.34 (1H, d), 7.21-7.11 (3H, m), 7.39 (1H, d), |
| | (CDCl₃, δ ppm): | 7.75 (1H, d), 7.95 (1H, dd), 8.13 (1H, d,)8.38 (1H, s) |
| 13-107 | ¹H-NMR (CDCl₃, δ ppm): | 1.49 (3H, d), 2.29 (3H, s), 2.75 (3H, s), 3.11-3.38 (2H, m), 4.59-4.63 (1H, m), 5.28 (2H, s), 6.61 (1H, d), 7.12-8.05 (8H, m) |
| 13-108 | ¹H-NMR | 1.3 (3H, d), 2.2 (3H, s), 3.0 (3H, s), 3.1-3.5 (2H, m), 4.3-4.4 (1H, m), 5.3 (2H, s), 6.6 (1H, s), 6.6-7.7 (7H, m), 8.7 (1H, d), 9.8 (1H, d) DMSO |
| | (CDCl₃, δ ppm): | |
| 13-109 | ¹H-NMR | 1.4 (3H, d), 2.3 (3H, s), 2.7 (3H, s), 3.1-3.3 (2H, m), 4.5-4.6 (1H, m), 5.5 (2H, s), 6.9 (1H, d), 7.0 (1H, s), 7.1-7.9 (6H, m), 8.2 (1H, s) |
| | (CDCl₃, δ ppm): | |
| 13-111 | ¹H-NMR | 1.47 (3H, d), 2.29 (3H, s), 2.74 (3H, s), 3.04-3.43 (2H, m), 4.54-4.75 (1H, m), 5.31 (2H, s), 6.63-8.18 (14H, m) |
| | (CDCl₃, δ ppm): | |
| 13-112 | ¹H-NMR | 1.49 (3H, d), 2.29 (3H, s), 2.73 (3H, s), 3.07-3.39 (2H, m), 4.51-4.70 (1H, m), 5.42 (2H, s), 6.54 (1H, d), 6.92-8.09 (12H, m) |
| | (CDCl₃, δ ppm): | |
| 13-115 | ¹H-NMR | 1.17 (3H, d), 2.29 (3H, s), 3.17 (3H, s), 3.30 (2H, d), 4.21-4.34 (1H, m), 5.41 (2H, s), 6.44 (1H, d), 6.91 (1H, s), 7.06-7.13 (2H, m), 7.40 (1H, |
| | (CDCl₃, δ ppm): | dd), 7.50 (1H, d), 7.70 (1H, d), 8.00 (1H, d), 8.48 (1H, bs) |
| 13-119 | ¹H-NMR | 1.09 (6H, t), 1.43 (3H, d), 2.28 (3H, s), 3.09-3.23 (4H, m), 4.53-4.67 (1H, m), 5.41 (2H, s), 6.84 (1H, d), 6.91 (1H, s), 6.04-6.73 (5H, m), 8.00 (1H, |
| | (CDCl₃, δ ppm): | d), 8.25 (1H, s) |
| 13-120 | ¹H-NMR | 1.53 (6H, s), 2.27 (3H, s), 5.05 (2H, s), 5.38 (2H, s), 6.74-8.32 (14H, m) |
| | (CDCl₃, δ ppm): | |
| 13-121 | ¹H-NMR | 1.58 (3H, s), 1.62 (6H, s), 2.29 (3H, s), 3.66 (3H, s), 3.75 (2H, d), 5.42 (2H, s), 6.27 (1H, s), 6.93-8.36 (9H, m) |
| | (CDCl₃, δ ppm): | |
| 13-122 | ¹H-NMR | 0.03-0.14 (4H, br m), 1.21 (6H, s), 2.19 (3H, s), 2.47 (1H, br), 5.56 (2H, s), 7.02-7.80 (8H, m), 8.64 (1H, s), 10.24 (1H, s) |
| | (DMSO-d₆, δ ppm): | |
| 13-124 | ¹H-NMR | 1.27 (3H, d), 1.98 (3H, s), 2.28 (3H, s), 2.52-2.61 (3H, m), 4.29-4.31 (1H, m), 5.98 (1H, s), 6.44 (1H, d), 7.25-7.27 (2H, m), 7.38-7.41 (1H, m), |
| | (CDCl₃, δ ppm): | 7.48-7.51 (1H, m), 7.65 (1H, d), 7.76 (1H, s), 7.94 (1H, d), 8.47 (1H, s) |
| 13-125 | ¹H-NMR | 1.25 (3H, d), 1.94 (3H, s), 2.35 (3H, s), 2.57-2.59 (2H, m), 4.28-4.32 (4H, m), 6.61 (1H, d), 7.40-7.50 (3H, m), 7.59-7.62 (2H, m), 7.66-7.68 (1H, |
| | (CDCl₃, δ ppm): | m), 8.05 (1H, s), 8.15 (1H, d), 8.50 (1H, s) |
| 13-127 | ¹H-NMR | 1.24 (3H, d), 1.95 (3H, s), 2.30 (3H, s), 2.49-2.66 (2H, m), 4.27-4.39 (1H, m), 5.29 (2H, s), 6.13 (1H, d), 6.52 (1H, bs), 7.06-7.16 (2H, m), |
| | (CDCl₃, δ ppm): | 7.33-7.44 (2H, m), 7.72 (1H, d), 7.79 (1H, d), 8.11 (1H, d), 8.36 (1H, bs) |
| 13-146 | ¹H-NMR | 1.25-1.31 (3H, m), 2.00-2.18 (6H, m), 2.48-2.86 (2H, m), 4.24-4.29 (1H, m), 5.14-5.27 (3H, m), 6.51-6.54 (1H, m), 7.04-7.17 (3H, m), |
| | (CDCl₃, δ ppm): | 7.47-7.62 (2H, m), 7.82-8.01 (2H, m), 8.49 (1H, s) |
| 13-147 | ¹H-NMR | 1.25-1.26 (3H, m), 1.93 (3H, s), 2.32 (3H, s), 2.56-2.60 (2H, m), 4.01-4.07 (3H, m), 4.32-4.32 (1H, m), 5.21-5.26 (2H, m), 6.17 (1H, d), |
| | (CDCl₃, δ ppm): | 7.16-7.22 (2H, m), 7.40-7.50 (2H, m), 7.79 (1H, d), 7.96-7.99 (1H, m), 8.19 (1H, d), 8.34 (1H, s) |
| 13-148 | ¹H-NMR | 0.85 (3H, t), 1.24 (3H, d), 1.90 (3H, s), 2.14-2.24 (1H, m), 2.28 (3H, s), 2.49-2.63 (4H, m), 4.30-4.32 (1H, m), 5.21-5.24 (1H, m), 6.10 (1H, d), |
| | (CDCl₃, δ ppm): | 6.84 (1H, s), 7.17-7.26 (4H, m), 7.78 (1H, d), 7.95-7.96 (1H, m), 8.09 (1H, d), 8.25 (1H, s) |
| 13-149 | ¹H-NMR (CDCl₃, δ ppm): | 1.2 (3H, d), 2.0 (3H, s), 2.5-2.7 (2H, m), 4.3-4.4 (1H, m), 5.4 (2H, s), 6.2 (1H, d), 6.9 (1H, s), 7.2-8.4 (6H, m), 8.6 (1H, s) |
| 13-150 | ¹H-NMR | 1.3 (3H, d), 2.0 (3H, s), 2.5-2.7 (2H, m), 4.3-4.4 (1H, m), 5.4 (2H, s), 6.2 (1H, d), 6.9 (1H, s), 7.2-8.4 (6H, m), 8.6 (1H, s) |
| | (CDCl₃, δ ppm): | |
| 13-158 | ¹H-NMR | 0.86 (3H, t), 1.43 (3H, d), 1.63 (3H, s), 2.25-2.70 (10H, m), 4.44-4.46 (1H, m), 5.22-5.25 (1H, m), 6.86 (1H, s), 7.19-7.27 (4H, m), 7.68-7.70 (1H, |
| | (CDCl₃, δ ppm): | m), 7.93-8.07 (2H, m), 8.21-8.24 (1H, m) |
| 13-166 | ¹H-NMR | 1.28 (3H, d), 1.93 (3H, s), 2.26 (3H, s), 2.52-2.54 (2H, m), 3.27-3.55 (1H, m), 4.27-4.29 (1H, m), 4.88-5.22 (3H, m), 6.28-6.31 (1H, m), |
| | (CDCl₃, δ ppm): | 6.54-6.57 (1H, m), 6.89-7.20 (4H, m), 7.73 (1H, d), 7.96 (1H, d), 8.04 (1H, d), 8.31 (1H, s) |
| 13-172 | ¹H-NMR | 1.63 (3H, d), 2.11 (3H, s), 2.40 (3H, s), 2.82-2.94 (2H, m), 4.75-4.94 (1H, m), 5.43 (2H, s), 6.91 (1H, s), 7.10-7.21 (2H, m), 7.28-7.39 (1H, m), |
| | (CDCl₃, δ ppm): | 7.55-7.73 (1H, m), 7.79-7.94 (2H, m), 8.76 (1H, d), 8.89-9.00 (1H, m) |
| 13-173 | ¹H-NMR | 0.97-1.0 (3H, d), 2.3 (3H, s), 2.6 (3H, s), 2.7-3.0 (2H, m), 4.2-4.3 (1H, m), 5.4 (2H, s), 6.1-6.2 (1H, d), 6.9 (1H, s), 7.1-8.0 (11H, m), 8.3 (1H, s) |
| | (CDCl₃, δ ppm): | |
| 13-192 | ¹H-NMR | 2.33 (3H, s), 5.44 (2H, s), 6.96 (1H, s), 7.09-7.14 (2H, m), 7.68 (1H, t), 7.87 (1H, d), 8.04 (1H, d), 8.19 (1H, d), 9.18 (1H, s) |
| | (DMSO-d₆, δ ppm): | |
| 13-193 | ¹H-NMR | 2.28 (3H, s), 2.95 (3H, d), 5.41 (2H, s), 6.32 (1H, d), 6.91 (1H, s), 7.01-7.12 (2H, m), 7.39 (1H, t), 7.50 (1H, d), 7.65 (1H, d), 7.87 (1H, d), |
| | (CDCl₃, δ ppm): | 8.40 (1H, s) |
| 13-194 | ¹H-NMR | 2.26 (3H, s), 2.78 (3H, d), 5.42 (2H, s), 6.92 (1H, d), 7.03-7.34 (6H, m), 7.59 (1H, d), 8.25 (1H, s) |
| | (CDCl₃, δ ppm): | |
| 13-195 | ¹H-NMR | 2.32 (3H, s), 2.97 (3H, d), 5.42 (2H, s), 6.02-6.07 (1H, m), 6.91 (1H, s), 7.13-7.16 (2H, m), 7.70 (1H, t), 7.99 (1H, d), 8.11 (1H, d), 8.27-8.30 (2H, |
| | (CDCl₃, δ ppm): | m) |
| 13-196 | ¹H-NMR | 1.94 (1H, t), 2.29 (3H, s), 4.18 (2H, dd), 5.40 (2H, s), 6.40-6.48 (1H, m), 6.91 (1H, s), 7.03-7.14 (2H, m), 7.45 (1H, dd), 7.55 (1H, d), 7.72 (1H, |
| | (CDCl₃, δ ppm): | d), 7.99 (1H, d), 8.19 (1H, bs) |
| 13-197 | ¹H-NMR | 2.17 (1H, t), 2.30 (3H, s), 4.15 (2H, dd), 5.42 (2H, s), 6.66-6.72 (1H, m), 6.91 (1H, s), 7.11-7.19 (2H, m), 7.38 (1H, bs), 7.44 (1H, dd), |
| | (CDCl₃, δ ppm): | 7.56 (1H, d), 7.61 (1H, d), 7.93 (1H, d) |
| 13-199 | ¹H-NMR | 1.29 (3H, d), 1.92 (3H, s), 2.03 (3H, s), 2.56-2.75 (2H, m), 3.76 (2H, s), 4.35-4.40 (1H, m), 5.43 (2H, s), 6.08 (1H, d), 6.91 (1H, s), 7.11 (1H, s), |
| | (CDCl₃, δ ppm): | 7.23 (1H, d), 7.43 (1H, t), 7.54 (1H, d), 7.64 (1H, d), 8.14 (1H, d), 8.80 (1H, s) |
| 13-203 | ¹H-NMR | 1.23 (3H, d), 1.93 (3H, s), 2.28 (3H, s), 2.49-2.64 (2H, m), 4.26-4.36 (1H, m), 5.49 (2H, s), 6.40 (1H, d), 6.98 (1H, s), 7.09-7.10 (2H, m), 7.39 (1H, |
| | (CDCl₃, δ ppm): | t), 7.50 (1H, d), 7.67 (1H, d), 8.00 (1H, d), 8.43 (1H, s) |
| 13-204 | ¹H-NMR | 1.23 (3H, d), 1.94 (3H, s), 2.28 (3H, s), 2.50-2.64 (2H, m), 3.10 (2H, q), 4.27-4.36 (1H, m), 5.49 (2H, s), 6.30 (1H, d), 6.81 (1H, s), 7.06-7.09 (2H, |
| | (CDCl₃, δ ppm): | m), 7.42 (1H, t), 7.52 (1H, d), 7.70 (1H, d), 8.03 (1H, d), 8.39 (1H, s) |
| 13-206 | ¹H-NMR | 1.25 (3H, d), 1.96 (3H, s), 2.32 (3H, s), 2.50-2.66 (2H, m), 4.28-4.40 (1H, m), 5.25 (2H, s), 6.25 (1H, d), 7.09-7.16 (2H, m), 7.43 (1H, dd), |
| | (CDCl₃, δ ppm): | 7.54 (1H, d), 7.72 (1H, d), 8.09 (1H, d), 8.42 (1H, bs) |
| 13-212 | ¹H-NMR (CDCl₃, δ ppm): | 1.26 (3H, d), 1.97 (3H, s), 2.34 (3H, s), 2.56-2.63 (2H, m), 4.30-4.39 (1H, m), 5.31 (2H, s), 6.23 (1H, d), 7.21-7.49 (7H, m), 7.56 (1H, d), 7.68 (1H, s), 7.75 (1H, d), 8.13-8.16 |
| | | (2H, m), 8.44 (1H, s) |
| 13-213 | ¹H-NMR | 1.19 (9H, s), 1.26 (3H, d), 1.98 (3H, s), 2.33 (3H, s), 2.56-2.64 (2H, m), 4.34-4.35 (1H, m), 5.20 (2H, s), 6.23 (1H, d), 7.17-7.19 (2H, m), 7.41 (1H, |
| | (CDCl₃, δ ppm): | s), 7.46 (1H, t), 7.56 (1H, d), 7.75 (1H, d), 8.11 (1H, d), 8.20 (1H, s), 8.42 (1H, s) |
| 13-214 | ¹H-NMR | 1.21 (3H, d), 1.95 (3H, s), 2.17 (3H, s), 2.49-2.65 (2H, m), 4.31-4.36 (1H, m), 5.31 (2H, s), 6.13 (1H, d), 7.09 (1H, d), 7.21 (1H, s), 7.31 (1H d), |
| | (CDCl₃, δ ppm): | 7.51 (1H, t), 7.59-7.62 (2H, m), 7.80 (1H, d), 7.96-7.99 (3H, m), 8.59 (1H, s), 8.70 (1H, s) |
| 13-215 | ¹H-NMR | 1.23 (3H, d), 1.95 (3H, s), 2.21 (3H, s), 2.51-2.66 (2H, m), 4.29-4.38 (1H, m), 5.30 (2H, s), 6.17 (1H, d), 7.12 (1H, d), 7.17 (1H, s), 7.46-7.66 (7H, |
| | (CDCl₃, δ ppm): | m), 7.78 (1H, d), 7.96 (1H, s), 8.38 (1H, s), 8.52 (1H, s) |
| 13-216 | ¹H-NMR | 1.24 (3H, d), 1.94 (3H, s), 2.19 (3H, s), 2.49-2.66 (2H, m), 4.31-4.35 (1H, m), 5.30 (2H, s), 6.15 (1H, d), 7.09 (1H, d), 7.19 (1H, s), 7.33-7.40 (3H, |
| | (CDCl₃, δ ppm): | m), 7.49 (1H, t), 7.59 (1H, d), 7.69-7.80 (3H, m), 7.95 (1H, s), 8.47 (1H, s), 8.55 (1H, s) |
| 13-217 | ¹H-NMR | 1.24 (3H, d), 1.96 (3H, s), 2.27 (3H, s), 2.51-2.66 (2H, m), 4.29-4.38 (1H, m), 5.29 (2H, s), 6.20 (1H, d), 7.10-7.16 (3H, m), 7.28-7.34 (2H, m), |
| | (CDCl₃, δ ppm): | 7.44-7.58 (4H, m), 7.77 (1H, d), 7.85 (1H, d), 7.94 (1H, s), 7.98 (1H, s), 8.47 (1H, s) |
| 13-218 | ¹H-NMR | 1.24 (3H, d), 1.96 (3H, s), 2.27 (3H, s), 2.52-2.66 (2H, m), 4.30-4.37 (1H, m), 5.30 (2H, s), 6.14 (1H, d), 7.14-7.16 (2H, m), 7.26-7.29 (2H, m), |
| | (CDCl₃, δ ppm): | 7.45-7.50 (3H, m), 7.58 (1H, d), 7.78 (1H, d), 7.88 (1H, d), 7.93-7.96 (2H, m), 8.46 (1H, s) |
| 13-219 | ¹H-NMR | 1.25 (3H, d), 1.97 (3H, s), 2.31-2.32 (6H, m), 2.52-2.66 (2H, m), 4.32-4.37 (1H, m), 5.29 (2H, s), 6.14 (1H, d), 7.12-7.18 (4H, m), 7.39-7.58 (4H, |
| | (CDCl₃, δ ppm): | m), 7.76-7.79 (2H, m), 7.92 (1H, s), 8.05 (1H, d), 8.43 (1H, s) |
| 13-221 | ¹H-NMR | 1.21 (3H, d), 1.94 (3H, s), 2.13 (3H, s), 2.49-2.66 (2H, m), 4.30-4.35 (1H, m), 5.30 (2H. S), 6.15 (1H, d), 7.04-7.11 (2H, m), 7.19 (1H, s), |
| | (CDCl₃, δ ppm): | 7.51 (1H, t), 7.61 (1H, d), 7.72 (1H, d), 7.80 (1H, d), 7.92-7.99 (3H, m), 8.61 (1H, s), 8.93 (1H, s) |
| 13-222 | ¹H-NMR | 1.23 (3H, d), 1.96 (3H, s), 2.25 (3H, s), 2.51-2.66 (2H, m), 3.78 (3H, s), 4.31-4.36 (1H, m), 5.29 (2H, s), 6.19 (1H, d), 6.83 (2H, d), 7.12-7.16 (2H, |
| | (CDCl₃, δ ppm): | m), 7.40 (2H, d), 7.46 (1H, t), 7.56 (1H, d), 7.75-7.79 (2H, m), 7.92 (2H, m), 8.48 (1H, s) |
| 13-223 | ¹H-NMR | 1.26 (3H, d), 1.98 (3H, s), 2.35 (3H, s), 2.52-2.66 (2H, m), 4.32-4.37 (1H, m), 5.36 (2H, s), 6.15 (1H, d), 7.22-7.27 (2H, m), 7.47 (1H, t), 7.56 (1H, |
| | (CDCl₃, δ ppm): | d), 7.72 (1H, s), 7.76 (1H, d), 7.84 (1H, s), 7.91 (2H, s), 8.17-8.21 (2H, m), 8.45 (1H, s) |
| 13-224 | ¹H-NMR | 1.25 (3H, d), 1.98 (3H, s), 2.33 (3H, s), 2.56-2.63 (2H, m), 4.33-4.37 (1H, m), 5.28 (2H, s), 6.15 (1H, d), 6.51 (1H, t), 7.08 (2H, d), 7.20-7.24 (2H, |
| | (CDCl₃, δ ppm): | m), 7.37-7.61 (7H, m), 7.77 (1H, d), 8.13 (1H, d), 8.39 (1H, s) |
| 13-225 | ¹H-NMR | 1.25 (3H, d), 1.98 (3H, s), 2.34 (3H, s), 2.51-2.66 (2H, m), 4.30-4.37 (1H, m), 5.32 (2H, s), 6.20 (1H, d), 7.22-7.23 (2H, m), 7.37-7.48 (6H, m), |
| | (CDCl₃, δ ppm): | 7.55 (1H, d), 7.69 (1H, s), 7.75 (1H, d), 8.10 (1H, s), 8.15 (1H, d), 8.42 (1H, s) |
| 13-233 | ¹H-NMR | 1.30 (3H, d), 2.01 (3H, s), 2.57-2.74 (2H, m), 4.33-4.42 (1H, m), 5.45 (2H, s), 6.26 (1H, d), 6.96 (1H, s), 7.38-7.51 (4H, m), 7.66 (1H, d), |
| | (CDCl₃, δ ppm): | 8.16 (1H, d), 9.17 (1H, s) |
| 13-234 | ¹H-NMR | 1.26 (3H, d), 1.99 (3H, s), 2.55-2.69 (2H, m), 4.33-4.42 (1H, m), 5.43 (2H, s), 6.15 (1H, d), 6.93 (1H, s), 7.00-7.08 (2H, m), 7.44 (1H, t), 7.55 (1H, |
| | (CDCl₃, δ ppm): | d), 7.70 (1H, d), 8.36 (1H, t), 8.69 (1H, s) |
| 13-235 | ¹H-NMR | 1.23 (3H, d), 2.00 (3H, s), 2.51-2.70 (8H, m), 4.27-4.36 (1H, m), 5.42 (2H, s), 6.05 (1H, d), 6.91 (1H, s), 7.05 (1H, d), 7.13 (1H, s), 7.43 (1H, t), |
| | (CDCl₃, δ ppm): | 7.52 (1H, s), 7.57 (1H, t), 8.39 (1H, d), 9.15 (1H, s) |
| 13-236 | ¹H-NMR (CDCl₃, δ ppm): | 1.22 (3H, d), 1.98 (3H, s), 2.49-2.68 (2H, m), 3.86 (3H, s), 4.30-4.36 (1H, m), 5.43 (2H, s), 6.11 (1H, d), 6.84-6.91 (3H, m), 7.42 (1H, t), 7.53 (1H, d), 7.63 (1H, d), 8.40 (1H, d), |
| | | 8.65 (1H, s) |
| 13-237 | ¹H-NMR | 1.26 (3H, d), 2.01 (3H, s), 2.38 (3H, s), 2.54-2.71 (2H, m), 4.30-4.39 (1H, m), 5.43 (2H, s), 6.07 (1H, d), 6.92 (1H, s), 7.21 (1H, d), 7.41-7.47 (2H, |
| | (CDCl₃, δ ppm): | m), 7.55 (1H, d), 7.63 (1H, d), 8.33 (1H, d), 8.97 (1H, s) |
| 13-240 | ¹H-NMR | 1.25 (3H, d), 1.93 (3H, s), 2.31 (3H, s), 2.52-2.65 (2H, m), 4.29-4.32 (1H, m), 5.26 (2H, s), 6.40 (1H, d), 7.11-7.21 (3H, m), 7.42 (1H, s), |
| | (CDCl₃, δ ppm): | 7.74 (1H, d), 7.94 (1H, d), 8.11 (1H, d), 8.39 (1H, s) |
| 13-243 | ¹H-NMR | 1.31 (3H, d), 2.00 (3H, s), 2.58-2.75 (2H, m), 4.31-4.40 (1H, m), 5.45 (2H, s), 6.13 (1H, d), 6.95 (1H, s), 7.19 (1H, t), 7.45-7.49 (2H, m), 7.72 (1H, |
| | (CDCl₃, δ ppm): | d), 7.94 (1H, d), 8.21 (1H, d), 9.04 (1H, s) |
| 13-244 | ¹H-NMR | 1.21-1.28 (3H, m), 1.94 (3H, s), 2.34 (3H, s), 2.51-2.65 (2H, m), 5.33 (2H, s), 6.28 (1H, d), 7.16-7.19 (2H, m), 7.64-7.74 (2H, m), 8.11-8.43 (4H, |
| | (CDCl₃, δ ppm): | m) |
| 13-245 | ¹H-NMR | 1.18 (3H, d), 1.96 (3H, s), 2.48-2.66 (8H, m), 4.23-4.28 (1H, m), 5.43 (2H, s), 6.22 (1H, d), 6.92 (1H, s), 7.07 (1H, d), 7.16 (1H, s), 7.67 (1H, t), |
| | (CDCl₃, δ ppm): | 7.95 (1H, d), 8.24 (1H, d), 8.43 (1H, d), 9.24 (1H, s) |
| 13-246 | ¹H-NMR | 1.21 (3H, d), 1.97 (3H, s), 2.39 (3H, s), 2.52-2.67 (2H, m), 4.25-4.34 (1H, m), 5.44 (2H, s), 6.19 (1H, d), 6.93 (1H, s), 7.23 (1H, d), 7.42 (1H, s), |
| | (CDCl₃, δ ppm): | 7.69 (1H, t), 7.99 (1H, d), 8.26 (1H, d), 8.38 (1H, d), 8.99 (1H, s) |
| 13-273 | ¹H-NMR | 0.92 (3H, t), 1.43-1.74 (3H, m), 1.90-2.10 (1H, m), 2.17-2.63 (8H, m), 4.21 (1H, br), 5.42 (2H, s), 6.91 (1H, s), 7.10-7.13 (2H, m), 7.69 (1H, t), |
| | (CDCl₃, δ ppm): | 8.12-8.16 (2H, m), 8.29 (1H, d), 8.44 (1H, s) |
| 13-291 | ¹H-NMR | 1.10 (3H, t), 1.20 (3H, d), 2.28 (3H, s), 2.39 (2H, q), 2.50 (3H, s), 2.52 (1H, dd), 2.66 (1H, dd), 4.21-4.36 (1H, m), 5.40 (2H, s), 6.41 (1H, d), |
| | (CDCl₃, δ ppm): | 6.91 (1H, s), 7.06-7.19 (2H, m), 7.36-7.56 (3H, m), 8.06 (1H, d), 8.34 (1H, bs) |
| 13-294 | ¹H-NMR | diastereomeric mixture1.22-1.49 (3H, m), 2.24-2.66 (6H, m), 2.73-2.91 (5H, m), 4.30-4.73 (1H, m), 5.33 (2H, s), 7.10-7.24 (2H, m), |
| | (CDCl₃, δ ppm): | 7.46-7.83 (4H, m), 8.00-8.25 (3H, m), |
| 13-295 | ¹H-NMR | 1.3 (3H/2, d), 1.4 (3H/2, d), 2.2-2.3 (6H, m), 2.6-2.8 (2H, m), 3.3 (3H, d), 4.5-4.6 (1H, m), 5.4 (2H, s), 6.9 (1H, s), 7.1-8.2 (8H, m) |
| | (CDCl₃, δ ppm): | |
| 13-296 | ¹H-NMR | 1.3 (3H, d), 2.3 (3H, s), 2.8 (3H, s), 3.3 (3H, s), 3.2-3.3 (2H, m), 4.6 (1H, m), 5.4 (2H, s), 6.8 (1H, d), 6.9 (1H, s), 7.1-8.2 (7H, m) |
| | (CDCl₃, δ ppm): | |
| 13-297 | ¹H-NMR | diastereomeric mixture1.14-1.47 (3H, m), 2.00-2.15 (3H, m), 2.22-2.40 (3H, m), 2.64-2.95 (5H, m), 4.20-4.59 (1H, m), 5.42 (2H, s), 6.91 (1H, |
| | (CDCl₃, δ ppm): | s), 7.04-7.20 (2H, m), 7.33-8.17 (5H, m), 8.47-8.67 (1H, m) |
| 13-299 | ¹H-NMR | 1.2 (3H, d), 1.9 (3H, s), 2.3 (3H, s), 2.5 (3H, s), 2.5-2.6 (2H, m), 4.3 (1H, m), 5.2 (2H, s), 6.2 (1H, s), 6.2-8.0 (6H, m), 8.3 (1H, d) |
| | (CDCl₃, δ ppm): | |
| 13-300 | ¹H-NMR | 1.2 (3H, d), 1.9 (3H, s), 2.3 (3H, s), 2.5 (3H, s), 2.5-2.6 (2H, m), 4.2-4.3 (1H, m), 5.2 (2H, s), 6.2 (1H, s), 6.2-8.0 (8H, m), 8.3 (1H, s) |
| | (CDCl₃, δ ppm): | |
| 13-301 | ¹H-NMR | 1.21 (3H, d), 1.93 (3H, s), 2.50 (1H, dd), 2.51 (3H, s), 2.61 (1H, dd), 4.27-4.39 (1H, m), 5.43 (2H, s), 6.13 (1H, d), 6.91 (1H, s), 7.19-7.27 (2H, |
| | (CDCl₃, δ ppm): | m), 7.38-7.49 (2H, m), 7.56-7.66 (3H, m), 8.66 (1H, bs) |
| 13-302 | ¹H-NMR (CDCl₃, δ ppm): | 1.23 (3H, d), 1.93 (3H, s), 1.94 (3H, d), 2.29 (3H, s), 2.48-2.66 (2H, m), 2.51 (3H, s), 4.27-4.38 (1H, m), 5.60 (1H, q), 6.16 (1H, d), 6.86 (1H, s), 7.12-7.22 (2H, m), 7.39-7.50 |
| | | (2H, m), 7.60 (1H, d), 8.09 (1H, d), 8.25 (1H, bs) |
| 13-303 | ¹H-NMR | 1.24 (3H, d), 1.96 (3H, s), 2.31 (3H, s), 2.51 (1H, dd), 2.52 (3H, s), 2.62 (1H, dd), 4.27-4.40 (1H, m), 5.28 (2H, s), 6.20 (1H, d), 7.10-7.18 (2H, |
| | (CDCl₃, δ ppm): | m), 7.39-7.52 (3H, m), 7.61 (1H, d), 8.18 (1H, d), 8.34 (1H, bs) |
| 13-306 | ¹H-NMR | 1.23 (3H, d), 1.98 (3H, s), 2.40 (3H, s), 2.53-2.67 (2H, m), 2.54 (3H, s), 4.28-4.43 (1H, m), 6.16 (1H, d), 7.06 (1H, s), 7.31-7.40 (2H, m), |
| | (CDCl₃, δ ppm): | 7.43-7.55 (2H, m), 7.65 (1H, d), 8.43-8.55 (2H, m) |
| 13-307 | ¹H-NMR | 1.23 (3H, d), 1.96 (3H, s), 2.31 (3H, s), 2.52 (3H, s), 2.50-2.66 (2H, m), 4.27-4.39 (1H, m), 5.25 (2H, s), 6.13 (1H, d), 7.08-7.16 (2H, m), |
| | (CDCl₃, δ ppm): | 7.42-7.63 (3H, m), 8.17 (1H, d), 8.28 (1H, bs) |
| 13-312 | ¹H-NMR | 0.21 (1H, m), 0.54 (1H, m), 1.02 (1H, m), 1.19 (3H, d), 2.04 (3H, s), 2.30 (3H, s), 2.52 (1H, dd), 2.61 (1H, dd), 2.89 (2H, d), 4.21 (1H, m), |
| | (CDCl₃, δ ppm): | 5.41 (2H, s), 6.39 (1H, d), 6.91 (1H, s), 7.12-7.60 (6H, m), 8.11 (1H, d) |
| 13-313 | ¹H-NMR | 1.18 (3H, s), 2.03 (3H, s), 2.29 (3H, s), 2.58 (1H, dd), 2.61 (1H, dd), 3.57 (2H, d), 4.23 (1H, m), 5.09 (2H, m), 5.42 (2H, s), 5.81 (1H, m), 6.40 (1H, |
| | (CDCl₃, δ ppm): | d), 6.91 (1H, s), 7.12 (1H, s), 7.17 (1H, d), 7.38-7.56 (4H, m), 8.10 (1H, d), |
| 13-314 | ¹H-NMR | 1.19 (3H, d), 2.02 (3H, s), 2.28 (3H, s), 2.49-2.63 (2H, m), 3.48-3.59 (2H, m), 4.21-4.30 (1H, m), 5.42 (2H, s), 6.32 (1H, d), 6.91 (1H, s), |
| | (CDCl₃, δ ppm): | 7.13-7.18 (2H, m), 7.41 (1H, s), 7.49 (1H, t), 7.65 (1H, d), 7.76 (1H, d), 8.06 (1H, d) |
| 13-315 | ¹H-NMR | 1.17 (3H, d), 2.01 (3H, s), 2.27 (3H, s), 2.48-2.61 (2H, m), 4.19-4.28 (1H, m), 5.42 (2H, s), 6.43 (1H, d), 6.92 (1H, s), 7.14-7.18 (2H, m), 7.54 (1H, |
| | (CDCl₃, δ ppm): | t), 7.63 (1H, s), 7.74 (1H, d), 7.86 (1H, d), 8.03 (1H, d) |
| 13-316 | ¹H-NMR | 0.26 (1H, m), 0.58 (1H, m), 1.05 (1H, m), 1.23 (3H, d), 1.94 (3H, s), 2.30 (3H, s), 2.45-2.65 (2H, m), 2.90 (2H, d), 4.32 (1H, m), 5.40 (2H, s), |
| | (CDCl₃, δ ppm): | 6.07 (1H, d), 6.90 (1H, s), 7.08-7.13 (2H, m), 7.40-7.70 (3H, m), 8.15 (1H, d), 8.31 (1H, bs) |
| 13-317 | ¹H-NMR | 1.03 (3H, t), 1.22 (3H, d), 1.62-1.76 (2H, m), 1.92 (3H, s), 2.29 (3H, s), 2.48-2.64 (2H, m), 2.94 (2H, t), 4.27-4.36 (1H, m), 5.40 (2H, s), 6.19 (1H, |
| | (CDCl₃, δ ppm): | d), 6.90 (1H, s), 7.09-7.12 (2H, m), 7.40-7.63 (3H, m), 8.11 (1H, d), 8.32 (1H, s) |
| 13-319 | ¹H-NMR | 1.04 (6H, d), 1.22 (3H, d), 1.85-1.96 (4H, m), 2.30 (3H, s), 2.48-2.64 (2H, m), 2.85 (2H, d), 4.28-4.37 (1H, m), 5.40 (2H, s), 6.12 (1H, d), |
| | (CDCl₃, δ ppm): | 6.90 (1H, s), 7.09-7.12 (2H, m), 7.42 (1H, t), 7.49 (1H, d), 7.61 (1H, d), 8.13 (1H, d), 8.30 (1H, s) |
| 13-320 | ¹H-NMR | 1.22 (3H, d), 1.88 (3H, s), 2.29 (3H, s), 2.45-2.63 (2H, m), 4.29-4.38 (1H, m), 5.41 (2H, s), 6.24 (1H, d), 6.91 (1H, s), 7.10-7.13 (2H, m), 7.57 (1H, |
| | (CDCl₃, δ ppm): | t), 7.87-7.94 (2H, m), 8.14 (1H, d), 8.28 (1H, s) |
| 13-321 | ¹H-NMR | 1.21 (3H, s), 1.91 (3H, s), 2.30 (3H, s), 2.46-2.63 (2H, m), 3.54 (2H, q), 4.26-4.35 (1H, m), 5.41 (2H, s), 6.11 (1H, d), 6.91 (1H, s), 7.10-7.14 (2H, |
| | (CDCl₃, δ ppm): | m), 7.50 (1H, t), 7.76 (1H, d), 7.80 (1H, d), 8.16 (1H, d), 8.20 (1H, s) |
| 13-322 | ¹H-NMR | 1.14 (3H, d), 1.88 (3H, s), 2.29 (3H, s), 2.37-2.58 (2H, m), 4.13-4.27 (3H, m), 5.40 (2H, s), 5.76 (1H, d), 6.90 (1H, s), 7.09-7.12 (2H, m), |
| | (CDCl₃, δ ppm): | 7.18-7.30 (5H, m), 7.39 (1H, t), 7.51 (1H, d), 7.68 (1H, d), 8.12 (1H, d), 8.34 (1H, s) |
| 13-326 | ¹H-NMR | 1.25 (3H, d), 1.98 (3H, s), 2.34 (3H, s), 2.49-2.63 (5H, m), 4.33-4.36 (1H, m), 5.35 (2H, s), 6.11 (1H, d), 7.21-7.26 (2H, m), 7.42-7.51 (2H, m), |
| | (CDCl₃, δ ppm): | 7.61-7.92 (5H, m), 8.21-8.25 (2H, m), 8.35 (1H, s) |
| 13-327 | ¹H-NMR | 1.19 (3H, d), 1.93 (3H, s), 2.34 (3H, s), 2.40-2.63 (2H, m), 3.29 (3H, s), 4.23-4.27 (1H, m), 5.37 (2H, s), 6.40 (1H, d), 7.23-7.29 (2H, m), |
| | (CDCl₃, δ ppm): | 7.69-7.74 (2H, m), 7.84 (1H, s), 7.90 (2H, s), 8.02-8.05 (2H, m), 8.19-8.22 (2H, m), 8.31 (1H, d) |
| 13-328 | ¹H-NMR (CDCl₃, δ ppm): | 1.24 (3H, d), 1.98 (3H, s), 2.33 (3H, s), 2.49-2.66 (5H, m), 4.3-4.36 (1H, m), 5.31 (2H, s), 6.13 (1H, d), 7.20-7.36 (6H, m), 7.43-7.51 (2H, m), 7.62 (1H, d), 7.67 (1H, s), |
| | | 8.12 (1H, s), 8.21 (1H, d), 8.33 (1H, s) |
| 13-329 | ¹H-NMR | 1.19 (9H, s), 1.24 (3H, d), 1.97 (3H, s), 2.32 (3H, s), 2.49-2.66 (5H, m), 4.28-4.38 (1H, m), 5.19 (2H, s), 6.18 (1H, d), 7.16-7.19 (2H, m), |
| | (CDCl₃, δ ppm): | 7.40-7.64 (4H, m), 8.15-8.20 (2H, m), 8.32 (1H, s) |
| 13-330 | ¹H-NMR | 1.24 (3H, d), 1.98 (3H, s), 2.32 (3H, s), 2.49-2.63 (5H, m), 4.29-4.38 (1H, m), 5.27 (2H, s), 6.15 (1H, d), 6.51 (1H, t), 7.08 (2H, d), 7.19-7.25 (2H, |
| | (CDCl₃, δ ppm): | m), 7.37-7.50 (5H, m), 7.57-7.63 (3H, m), 8.18 (1H, d), 8.30 (1H, s) |
| 13-331 | ¹H-NMR | 1.19 (3H, d), 1.93 (3H, s), 2.31 (3H, s), 2.41-2.65 (2H, m), 3.29 (3H, s), 4.24-4.28 (1H, m), 5.29 (2H, s), 6.34 (1H, d), 6.51 (1H, t), 7.08 (2H, d), |
| | (CDCl₃, δ ppm): | 7.17-7.24 (2H, m), 7.37-7.74 (6H, m), 7.96 (1H, s), 8.05 (1H, d), 8.20-8.29 (2H, m) |
| 13-332 | ¹H-NMR | 1.20 (3H, d), 1.93 (3H, s), 2.15 (3H, s), 2.46-2.66 (5H, m), 4.24-4.38 (1H, m), 5.27 (2H, s), 6.27 (1H, d), 7.06-7.24 (5H, m), 7.45-7.61 (6H, m), |
| | (CDCl₃, δ ppm): | 7.94 (1H, s), 8.42-8.46 (2H, m) |
| 13-333 | ¹H-NMR | 1.19 (3H, d), 1.92 (3H, s), 2.30 (3H, s), 2.41-2.63 (2H, m), 3.28 (3H, s), 4.21-4.29 (1H, m), 5.32 (2H, s), 6.42 (1H, d), 7.18-7.21 (2H, m), 7.72 (1H, |
| | (CDCl₃, δ ppm): | t), 7.81 (1H, d), 7.93-7.98 (3H, m), 8.03-8.06 (2H, m), 8.13-8.23 (3H, m) |
| 13-334 | ¹H-NMR | 1.19 (3H, d), 1.92 (3H, s), 2.30 (3H, s), 2.41-2.63 (2H, m), 3.29 (3H, s), 4.21-4.30 (1H, m), 5.33 (2H, s), 6.37 (1H, d), 7.18-7.22 (2H, m), 7.64 (1H, |
| | (CDCl₃, δ ppm): | s), 7.73 (1H, t), 7.96 (1H, s), 8.03-8.09 (5H, m), 8.18-8.24 (2H, m) |
| 13-335 | ¹H-NMR | 1.20 (3H, d), 1.94 (3H, s), 2.14 (3H, s), 2.46-2.67 (5H, m), 4.27-4.34 (1H, m), 5.29 (2H, s), 6.15 (1H, d), 7.06 (1H, d), 7.18 (1H, s), 7.32-7.35 (3H, |
| | (CDCl₃, δ ppm): | m), 7.45-7.53 (2H, m), 7.63-7.73 (3H, m), 7.96 (1H, s), 8.50 (1H, s), 8.70 (1H, s) |
| 13-336 | ¹H-NMR | 1.21 (3H, d), 1.94 (3H, s), 2.10 (3H, s), 2.48-2.67 (5H, m), 4.26-4.35 (1H, m), 5.28 (2H, s), 6.20 (1H, d), 7.05 (1H, d), 7.17-7.30 (2H, m) |
| | (CDCl₃, δ ppm): | 7.45-7.64 (7H, m), 7.98 (1H, s), 8.49 (1H, s), 8.78 (1H, s) |
| 13-337 | ¹H-NMR | 1.22 (3H, d), 1.95 (3H, s), 2.24 (3H, s), 2.30 (3H, s), 2.48-2.66 (5H, m), 4.30-4.34 (1H, m), 5.28 (2H, s), 6.19 (1H, d), 7.08-7.20 (4H, m), |
| | (CDCl₃, δ ppm): | 7.36-7.50 (4H, m), 7.60-7.79 (2H, m), 7.93 (1H, s), 8.01 (1H, s), 8.39 (1H, s) |
| 13-338 | ¹H-NMR | 1.20 (3H, d), 1.94 (3H, s), 2.09 (3H, s), 2.53-2.67 (5H, m), 4.21-4.30 (1H, m), 5.27 (2H, s), 6.22 (1H, d), 7.05-7.19 (5H, m), 7.40-7.64 (5H, m). |
| | (CDCl₃, δ ppm): | 7.94 (1H, s), 8.50 (1H, s), 8.68 (1H, s) |
| 13-339 | ¹H-NMR | 1.23 (3H, d), 1.97 (3H, s), 2.28 (3H, s), 2.94-2.66 (5H, m), 3.79 (3H, s), 4.31-4.35 (1H, m), 5.29 (2H, s), 6.12 (1H, d), 6.85 (2H, d), 7.15-7.17 (2H, |
| | (CDCl₃, δ ppm): | m), 7.41-7.51 (4H, m), 7.63 (1H, d), 7.79 (1H, s), 7.91 (1H, s), 8.01 (1H, d), 8.34 (1H, s) |
| 13-344 | ¹H-NMR | 1.06 (3H, d), 1.88 (3H, s), 2.24 (3H, s), 2.11-2.64 (2H, m), 2.75 (6H, s), 3.84-4.01 (1H, m), 5.55 (2H, s), 7.04 (1H, d), 7.11 (1H, s), |
| | (DMSO-d₆, δ ppm): | 7.57-7.98 (5H, m), 8.34 (1H, d), 9.40 (1H, bs) |
| 13-347 | ¹H-NMR | 1.19 (3H, d), 1.98 (3H, s), 2.46-2.68 (5H, m), 3.84 (3H, s), 4.25-4.34 (1H, m), 5.42 (2H, s), 6.12 (1H, d), 6.83-6.90 (2H, m), 6.91 (1H, s), |
| | (CDCl₃, δ ppm): | 7.38-7.49 (3H, m), 8.42 (1H, d), 8.56 (1H, s) |
| 13-348 | ¹H-NMR | 1.27 (3H, d), 1.92 (3H, s), 2.02 (3H, s), 2.51 (3H, s), 2.54-2.75 (2H, m), 3.75 (2H, s), 4.31-4.40 (1H, m), 5.42 (2H, s), 6.11 (1H, d), 6.91 (1H, s), |
| | (CDCl₃, δ ppm): | 7.10-7.11 (1H, m), 7.21-7.24 (1H, m), 7.40-7.51 (3H, m), 8.16 (1H, d), 8.72 (1H, s) |
| 13-350 | ¹H-NMR | 1.39 (3H, d), 2.16 (3H, s), 2.50 (3H, s), 2.69-2.88 (2H, m), 4.39-4.48 (1H, m), 6.26 (1H, d), 6.66 (1H, d), 7.09 (1H, d), 7.26-7.39 (3H, m), 7.78 (1H, |
| | (CDCl₃, δ ppm): | s), 7.95-8.04 (2H, m), 10.53 (1H, s) |
| 13-352 | ¹H-NMR (CDCl₃, δ ppm): | 1.24 (3H, d), 1.96 (3H, s), 2.44 (3H, s), 2.58 (2H, d), 4.30-4.40 (1H, m), 6.15 (1H, d), 7.22 (1H, s), 7.49 (1H, dd), 7.60 (1H, d), 7.79 (1H, d), 8.18 (1H, s), 8.25 |
| | | (1H, d), 8.53 (1H, d), 8.68 (1H, bs) |
| 13-355 | ¹H-NMR | 1.20 (3H, d), 1.92 (3H, s), 2.31 (3H, s), 2.41-2.63 (2H, m), 3.29 (3H, s), 4.24-4.28 (1H, m), 5.31 (2H, s), 6.36 (1H, d), 7.18-7.23 (2H, m), 7.74 (1H, |
| | (CDCl₃, δ ppm): | dd), 7.81 (1H, d), 7.95-8.07 (6H, m), 8.20-8.25 (2H, m) |
| 13-356 | ¹H-NMR | 1.08-1.20 (9H, m), 1.68 (3H, s), 1.93 (3H, s), 2.29-2.36 (4H, m), 2.47-2.65 (2H, m), 3.29 (3H, s), 4.21-4.30 (1H, m), 5.27 (2H, s), 6.10 (1H, bs), |
| | (CDCl₃, δ ppm): | 6.427 (1H, d), 7.14 (1H, s), 7.20 (1H, d), 7.71 (1H, dd), 7.97-8.05 (3H, m), 8.19-8.30 (2H, m) |
| 13-357 | ¹H-NMR | 1.19 (3H, d), 1.94 (3H, s), 2.28 (3H, s), 2.41-2.65 (2H, m), 3.30 (3H, s), 3.45 (3H, s), 4.23-4.28 (1H, m), 5.14 (2H, s), 6.34 (1H, d), 6.98-7.03 (3H, |
| | (CDCl₃, δ ppm): | m), 7.52 (2H, s), 7.72-7.77 (2H, m), 7.98 (1H, s), 8.07 (1H, d), 8.22-8.27 (2H, m) |
| 13-358 | ¹H-NMR | 1.19 (3H, d), 1.92 (3H, s), 2.29 (3H, s), 2.41-2.63 (2H, m), 3.29 (3H, s), 4.23-4.28 (1H, m), 5.31 (2H, s), 6.39 (1H, d), 7.19-7.23 (2H, m), 7.63 (1H, |
| | (CDCl₃, δ ppm): | s), 7.74 (1H, dd), 7.96 (1H, s), 8.03-8.24 (7H, m) |
| 13-360 | ¹H-NMR | 1.23 (3H, d), 1.95 (3H, s), 2.12 (3H, s), 2.48-2.68 (5H, m), 4.30-4.35 (1H, m), 5.28 (2H, s), 6.14 (1H, d), 7.08-7.17 (2H, m), 7.28-7.31 (1H, m), |
| | (CDCl₃, δ ppm): | 7.49-7.54 (2H, m), 7.64-7.73 (2H, m), 7.93-7.95 (2H, m), 8.00 (1H, s), 8.48 (1H, bs), 8.88 (1H, bs) |
| 13-361 | ¹H-NMR | 1.24 (3H, d), 1.95 (3H, s), 2.15 (3H, s), 2.56-2.63 (2H, m), 4.31-4.36 (1H, m), 5.29 (2H, s), 6.15 (1H, d), 7.10 (1H, d), 7.18 (1H, s), 7.35 (1H, d), |
| | (CDCl₃, δ ppm): | 7.47-7.62 (2H, m), 7.72-7.80 (2H, m), 7.93-7.99 (3H, m), 8.58 (1H, bs), 8.73 (1H, bs) |
| 13-362 | ¹H-NMR | 1.26 (3H, d), 2.04 (3H, s), 2.51-2.78 (2H, m), 3.76 (2H, s), 4.24-4.36 (1H, m), 6.27 (1H, bs), 6.42 (1H, d), 6.97-6.99 (3H, m), 7.28-7.32 (1H, m), |
| | (CDCl₃, δ ppm): | 7.40-7.46 (6H, m), 7.58-7.65 (2H, m), 10.00 (1H, bs) |
| 13-365 | ¹H-NMR | 1.24 (3H, d), 1.95 (3H, s), 2.45 (3H, s), 2.58 (2H, d), 4.30-4.41 (1H, m), 5.76 (2H, s), 6.12 (1H, d), 7.00 (1H, s), 7.50 (1H, dd), 7.60 (1H, d), |
| | (CDCl₃, δ ppm): | 7.78-7.85 (3H, m), 8.53 (1H, d), 8.68 (1H, bs) |
| 13-366 | ¹H-NMR | 1.26 (3H, d), 2.01 (3H, s), 2.34 (3H, s), 2.57 (1H, dd), 2.65 (1H, dd), 4.30-4.44 (3H, m), 5.22-5.31 (1H, m), 6.14 (1H, d), 6.92 (1H, s), |
| | (CDCl₃, δ ppm): | 7.21-7.37 (2H, m), 7.47 (1H, dd), 7.56 (1H, d), 7.77 (1H, d), 8.10 (1H, d), 8.38 (1H, bs) |
| 13-378 | ¹H-NMR | 1.53 (6H, s), 2.10 (3H, s), 2.32 (3H, s), 3.04 (2H, s), 5.42 (2H, s), 6.90 (1H, s), 7.08-7.18 (3H, br), 7.47-7.59 (1H, m), 7.92-8.43 (3H, m), 8.64 (1H, |
| | (CDCl₃, δ ppm): | d) |
| 13-501 | ¹H-NMR | 1.25 (3H, d), 1.85 (3H, s), 2.28 (3H, s), 2.43-2.80 (2H, m), 4.14-4.18 (5H, m), 5.79 (2H, s), 7.14-7.25 (4H, m), 7.66-7.74 (2H, m), |
| | (Acetone-d₆, δ ppm): | 7.93-8.00 (2H, m), 8.20-8.23 (1H, m), 8.45-8.48 (1H, m), 8.73 (1H, br s) |
| 13-507 | ¹H-NMR | 1.25 (3H, d), 1.92 (3H, s), 2.15 (3H, s), 2.54 (1H, dd), 2.62 (1H, dd), 4.23-4.37 (1H, m), 5.34 (2H, s), 6.47 (1H, d), 7.12-7.22 (3H, m), 7.72 (1H, |
| | (CDCl₃, δ ppm): | d), 7.93 (1H, d), 8.04 (1H, s), 8.17 (1H, d), 8.46 (1H, s) |
| 14-17 | ¹H-NMR | 1.21 (3H, d), 1.98 (3H, s), 2.31 (3H, s), 2.51-2.67 (2H, m), 3.88 (3H, s), 4.26-4.39 (1H, m), 5.48 (2H, s), 6.21 (1H, d), 7.07 (1H, d), 7.12-7.21 (2H, |
| | (CDCl₃, δ ppm): | m), 7.37-7.50 (2H, m), 8.17 (1H, d), 8.41 (1H, bs) |
| 14-20 | ¹H-NMR | 1.3 (3H, d), 2.0 (3H, s), 2.6-2.7 (2H, m), 4.3-4.4 (1H, m), 5.5 (2H, s), 6.1 (1H, d), 7.3-8.5 (6H, m), 8.6 (1H, s) |
| | (CDCl₃, δ ppm): | |
| 14-21 | ¹H-NMR | 1.25 (3H, d), 1.94 (3H, s), 2.33 (3H, s), 2.48-2.66 (2H, m), 4.25-4.41 (1H, m), 5.49 (2H, s), 6.15 (1H, d), 7.16-8.42 (7H, m) |
| | (CDCl₃, δ ppm): | |
| 14-22 | ¹H-NMR (CDCl₃, δ ppm): | 1.24 (3H, d), 1.94 (3H, s), 2.33 (3H, s), 2.57 (2H, m), 4.34 (1H, m), 5.50 (2H, s), 6.11 (1H, d), 7.21 (2H, m), 7.44-7.58 (2H, m), 7.76 (1H, d), 8.17 (1H, d), 8.41 (1H, s). |
| 14-23 | ¹H-NMR | 1.23 (3H, d), 1.97 (3H, s), 2.32 (3H, s), 2.58 (2H, m), 3.61 (2H, q), 4.29-4.39 (1H, m), 5.39 (2H, s), 6.15 (1H, d), 7.07 (2H, t), 7.50 (2H, m), |
| | (CDCl₃, δ ppm): | 7.75 (1H, d), 8.16 (1H, d), 8.43 (1H, s). |
| 14-24 | ¹H-NMR | 1.26 (3H, d), 1.97 (3H, s), 2.42 (3H, s), 2.61 (2H, d), 4.32-4.41 (1H, m), 6.19 (1H, d), 7.34-7.61 (4H, m), 7.79 (1H, dd), 8.49 (1H, d), 8.70 (1H, s). |
| | (CDCl₃, δ ppm): | |
| 14-30 | ¹H-NMR | 1.23 (3H, d), 1.94 (3H, s), 2.32 (3H, s), 2.50-2.66 (2H, m), 4.25-4.39 (1H, m), 5.48 (2H, s), 6.15 (1H, d), 7.16-8.41 (7H, m) |
| | (CDCl₃, δ ppm): | |
| 14-32 | ¹H-NMR | 1.25 (3H, d), 1.96 (3H, s), 2.33 (3H, s), 2.52-2.71 (2H, m), 4.30-4.39 (1H, m), 5.35 (2H, s), 6.18 (1H, d), 7.16-8.45 (8H, m) |
| | (CDCl₃, δ ppm): | |
| 14-33 | ¹H-NMR | 1.32 (3H, d), 2.27 (3H, s), 2.35 (3H, s), 2.67-2.90 (2H, m), 4.34-4.52 (1H, m), 5.49 (2H, s), 7.02 (1H, d), 7.13-7.23 (2H, m), 7.45-7.78 (4H, m), |
| | (CD₃CN, δ ppm): | 8.28-8.38 (1H, m), 8.43 (1H, s) |
| 14-34 | ¹H-NMR | 1.35 (3H, d), 2.27 (3H, s), 2.82 (3H, s), 3.07 (1H, dd), 3.36 (1H, dd), 4.43-4.54 (1H, m), 5.49 (2H, s), 7.00 (1H, d), 7.18 (1H, d), 7.23 (1H, s), |
| | (CD₃CN, δ ppm): | 7.44-7.72 (4H, m), 8.27 (1H, bs), 8.43 (1H, s) |
| 14-35 | ¹H-NMR | 1.24 (3H, d), 1.93 (3H, s), 2.32 (3H, s), 2.51-2.63 (2H, m), 4.31-4.38 (1H, m), 5.51 (2H, s), 6.18 (1H, d), 7.14-8.42 (7H, m) |
| | (CDCl₃, δ ppm): | |
| 14-36 | ¹H-NMR | 1.24 (3H, d), 1.49 (3H, s), 2.32 (3H, s), 2.51-2.64 (2H, m), 4.29-4.38 (1H, m), 5.63 (2H, s), 6.17 (1H, d), 6.87-8.41 (8H, m) |
| | (CDCl₃, δ ppm): | |
| 14-37 | ¹H-NMR | 1.24 (3H, d), 1.94 (3H, s), 2.30 (3H, s), 2.51-2.65 (2H, m), 2.71 (3H, s), 4.31-4.36 (1H, m), 5.22 (2H, s), 6.20 (1H, d), 7.11-8.39 (7H, m) |
| | (CDCl₃, δ ppm): | |
| 14-41 | ¹H-NMR | 1.26 (3H.d), 1.94 (3H, s), 2.29 (3H, s), 2.52-2.66 (2H, m), 4.30-4.39 (1H, m), 5.52 (2H, s), 6.68 (1H, d), 7.15-8.57 (7H, m) |
| | (CDCl₃, δ ppm): | |
| 14-42 | ¹H-NMR | 1.24 (3H, d), 1.95 (3H, s), 2.31 (3H, s), 2.51-2.65 (2H, m), 3.89-4.00 (2H, m), 4.29-4.38 (1H, m), 5.29 (2H, s), 6.15 (1H, d), 7.11-8.39 (7H, m) |
| | (CDCl₃, δ ppm): | |
| 14-43 | ¹H-NMR | 1.24 (3H, d), 1.93 (3H, s), 2.32 (3H, s), 2.51-2.64 (2H, m), 4.31-4.38 (1H, m), 5.50 (2H, s), 6.18 (1H, d), 7.14-8.42 (7H, m) |
| | (CDCl₃, δ ppm): | |
| 14-44 | ¹H-NMR | 1.22 (3H, d), 1.40 (9H, s), 1.95 (3H, s), 2.27 (3H, s9, 3.5-3.65 (2H, m), 4.25-4.40 (1H, m), 5.48 (2H, s), 6.14 (1H, d), 6.87-8.37 (7H, m) |
| | (CDCl₃, δ ppm): | |
| 14-45 | ¹H-NMR | 1.25 (3H, d), 2.01 (3H, s), 2.43 (3H, s), 2.61 (2H, d), 4.37 (1H, m), 6.15 (1H, d), 7.48 (4H, m), 7.80 (1H, d), 8.51 (1H, d), 8.67 (1H, s). |
| | (CDCl₃, δ ppm): | |
| 14-46 | ¹H-NMR | 1.25 (3H, d), 1.95 (3H, s), 2.29 (3H, s), 2.29-2.68 (2, m), 4.25-4.43 (1H, m), 5.42 (2H, s), 6.20 (1H, d), 6.98-8.42 (11H, m) |
| | (CDCl₃, δ ppm): | |
| 14-48 | ¹H-NMR (CDCl₃, δ ppm): | 1.22 (3H, d), 1.93 (3H, s), 2.26 (3H, s), 2.49-2.63 (2H, m), 4.20-4.38 (1H, m), 6.06 (2H, s), 6.19 (1H, d), 7.15-8.67 (10H, m) |
| 14-49 | ¹H-NMR | 1.21 (3H, d), 1.87 (3H, s), 2.24 (3H, s), 2.45-2.61 (2H, m), 4.26-4.35 (1H, m), 4.53 (2H, s), 6.63 (1H, d), 7.17-8.99 (10H, m) |
| | (CDCl₃, δ ppm): | |
| 14-50 | ¹H-NMR | 1.20 (3H, d), 1.91 (3H, s), 2.21 (3H, s), 2.47-2.63 (2H, m), 4.18-4.36 (1H, m), 6.50 (2H, s), 6.50-8.66 (11H, m) |
| | (CDCl₃, δ ppm): | |
| 14-51 | ¹H-NMR | 1.20 (3H, d), 1.94 (3H, s), 2.28 (3H, s), 2.50-2.64 (2H, m), 4.21-4.36 (1H, m), 5.68 (2H, s), 6.17 (1H, d), 6.59-8.36 (10H, m) |
| | (CDCl₃, δ ppm): | |
| 14-52 | ¹H-NMR | 1.22 (3H, d), 1.95 (3H, s), 2.29 (3H, s), 2.50-2.62 (2H, m), 4.24-4.40 (1H, m), 5.57 (2H, s), 6.24 (1H, d), 6.99-8.41 (10H, m) |
| | (CDCl₃, δ ppm): | |
| 14-54 | ¹H-NMR | 1.40 (3H, d), 2.42 (6H, s), 2.88 (2H, d), 4.54 (1H, mz), 6.93 (1H, d), 7.36-7.76 (5H, m), 8.50 (2H, m). |
| | (CDCl₃, δ ppm): | |
| 14-63 | ¹H-NMR | 1.51 (3H, d), 2.42 (3H, s), 2.86 (3H, s), 3.20-3.36 (2H, m), 4.66 (1H, m), 6.66 (1H, d), 7.55 (5H, m), 8.05 (1H, s), 8.33 (1H, m). |
| | (CDCl₃, δ ppm): | |
| 14-66 | ¹H-NMR | 1.47 (3H, d), 2.30 (3H, s), 2.78 (3H, s), 3.11-3.38 (2H, m), 4.56-4.62 (1H, m), 5.51 (2H, s), 6.75 (1H, d), 7.16-8.13 (7H, m) |
| | (CDCl₃, δ ppm): | |
| 14-67 | ¹H-NMR | 1.50 (3H, d), 2.42 (3H, s), 2.85 (3H, s), 3.29 (2H, ddd), 4.62-4.71 (1H, m), 6.69 (1H, d), 7.37-7.72 (5H, m), 8.36 (2H, d). |
| | (CDCl₃, δ ppm): | |
| 14-68 | ¹H-NMR | 1.49 (3H, d), 2.28 (3H, s), 2.80 (3H, s), 3.05-3.39 (2H, m), 4.52-4.68 (1H, m), 5.42 (2H, s), 6.59-8.04 (12H, m) |
| | (CDCl₃, δ ppm): | |
| 14-69 | ¹H-NMR | 1.09 (3H, t), 1.20 (3H, d), 1.59 (3H, s), 2.32 (3H, s), 2.34-2.64 (2H, m), 2.50-2.71 (2H, m), 4.21-4.39 (1H, m), 5.49 (2H, m), 6.19 (1H, d), |
| | (CDCl₃, δ ppm): | 7.16-8.43 (7H, m) |
| 14-71 | ¹H-NMR | 1.09 (3H, t), 1.18 (3H, d), 1.96 (3H, d), 2.32 (3H, s), 2.38 (2H, m), 2.61 (2H, m), 4.31 (1H, m), 5.73 (1H, d), 6.26 (1H, d), 7.23 (2H, m), |
| | (CDCl₃, δ ppm): | 7.41-7.55 (3H, m), 7.73 (1H, d), 8.09 (1H, d), 8.45 (1H, s) |
| 14-72 | ¹H-NMR | 1.11 (3H, t), 1.21 (3H, d), 2.32 (3H, s), 2.34-2.45 (2H, m), 2.50-2.71 (2H, m), 4.23-4.29 (1H, m), 5.48 (2H, s), 6.25 (1H, d), 7.16-8.44 (7H, m) |
| | (CDCl₃, δ ppm): | |
| 14-73 | ¹H-NMR | 1.07 (3H, t), 1.20 (3H, d), 2.30 (3H, s), 2.27-2.43 (2H, m), 2.50-2.71 (2H, m), 4.21-4.41 (1H, m), 5.52 (2H, s), 6.0-6.61 (3H, m), 7.16-8.41 (7H, m) |
| | (CDCl₃, δ ppm): | |
| 14-74 | ¹H-NMR | 1.11 (3H, t), 1.19 (3H, d), 2.32 (3H, s), 2.34-2.46 (2H, m), 2.53-2.70 (2H, m), 4.27-4.36 (1H, m), 5.51 (2H, s), 6.18 (1H, d), 7.15-8.14 (7H, m) |
| | (CDCl₃, δ ppm): | |
| 14-75 | ¹H-NMR | 1.12 (3H, t), 1.42 (3H, d), 2.32 (3H, s), 2.50 (2H, m), 2.78 (2H, d), 4.49 (1H, m), 5.50 (2H, s), 6.90 (1H, d), 7.18 (2H, m), 7.40-7.56 (2H, m), |
| | (CDCl₃, δ ppm): | 7.68 (1H, m), 8.07 (1H, m), 8.39 (1H, s). |
| 14-77 | ¹H-NMR (CDCl₃, δ ppm): | 1.0 (3H, t), 1.27 (3H, d), 2.26 (3H, s), 2.29-2.45 (2H, m), 2.63-2.80 (2H, m), 4.29-4.46 (1H, m), 5.52 (2H, s), 6.0-6.63 (2H, m), 7.11-8.65 (8H, m) |
| 14-78 | ¹H-NMR | 1.24 (3H, t), 1.48 (3H, d), 2.29 (3H, s), 2.86-2.93 (2H, m), 3.19 (2H, ddd), 4.57-4.66 (1H, m), 5.48 (2H, s), 6.65 (1H, d), 7.19 (2H, m), |
| | (CDCl₃, δ ppm): | 7.42-7.56 (2H, m), 7.64 (1H, m), 8.06 (2H, m). |
| 14-80 | ¹H-NMR | 1.22 (3H, t), 1.48 (3H, d), 2.31 (3H, s), 2.76-2.93 (2H, m), 3.04-3.32 (2H, m), 4.54-4.68 (1H, m), 5.23 (2H, s), 6.0-6.64 (3H, m), 7.17-8.05 (7H, m) |
| | (CDCl₃, δ ppm): | |
| 14-81 | ¹H-NMR | 1.39 (6H, s), 1.95 (3H, s), 2.33 (3H, s), 2.84 (2H, s), 5.47 (2H, s), 6.04 (1H, s), 7.14-8.47 (7H, m) |
| | (CDCl₃, δ ppm): | |
| 14-82 | ¹H-NMR | 1.24 (3H, d), 1.94 (3H, s), 2.33 (3H, s), 2.55 (1H, dd), 2.61 (1H, dd), 4.27-4.41 (1H, m), 5.46 (2H, s), 6.14 (1H, d), 7.15 (1H, s), 7.19 (1H, d), |
| | (CDCl₃, δ ppm): | 7.46 (1H, dd), 7.56 (1H, d), 7.75 (1H, d), 8.17 (1H, d), 8.42 (1H, bs) |
| 14-97 | ¹H-NMR | 1.22 (3H, d), 1.92 (3H, s), 2.31 (3H, s), 2.52 (1H, dd), 2.59 (1H, dd), 4.24-4.38 (1H, m), 5.49 (2H, s), 6.39-6.47 (1H, m), 7.14 (1H, s), 7.16 (1H, d), |
| | (CDCl₃, δ ppm): | 7.39 (1H, dd), 7.49 (1H, d), 7.65 (1H, d), 8.05 (1H, d), 8.50 (1H, bs) |
| 14-112 | ¹H-NMR | 1.40 (6H, s), 1.95 (3H, sz), 2.33 (3H, s), 2.84 (2H, s), 5.49 (2H, s), 6.06 (1H, s), 7.15-7.24 (2H, m), 7.42-7.57 (2H, m), 7.75 (1H, d), 8.25 (1H, d), |
| | (CDCl₃, δ ppm): | 8.48 (1H, s) |
| 14-114 | ¹H-NMR | 1.39 (6H, s), 1.95 (3H, s), 2.32 (3H, s), 2.84 (2H, s), 5.51 (2H, s), 6.05 (1H, s), 7.13-8.47 (7H, m) |
| | (CDCl₃, δ ppm): | |
| 14-115 | ¹H-NMR | 1.57 (6H, d), 2.26 (3H, s), 2.31 (3H, s), 3.0 (2H, dd), 5.50 (2H, s), 6.75 (1H, s), 7.15-7.22 (2H, m), 7.41-7.56 (2H, m), 7.70 (1H, d), 8.18 (1H, d), |
| | (CDCl₃, δ ppm): | 8.38 (1H, s) |
| 14-116 | ¹H-NMR | 1.63 (6H, s), 2.33 (3H, s), 2.60 (3H, s), 3.54 (2H, s), 5.49 (2H, s), 6.40 (1H, s), 7.16-7.30 (2H, m), 7.38-7.59 (2H, m), 7.68 (1H, d), 7.93-7.96 (1H, |
| | (CDCl₃, δ ppm): | m), 8.18 (1H, m). |
| 14-117 | ¹H-NMR | 1.11 (3H, t), 1.47 (6H, s), 2.36 (3H, s), 2.37 (2H, q), 2.85 (2H, s), 5.47 (2H, s), 6.09 (1H, s), 7.18 (2H, m), 7.37-7.61 (2H, m), 7.76 (1H, d), |
| | (CDCl₃, δ ppm): | 8.25 (1H, d), 8.50 (1H, s). |
| 14-118 | ¹H-NMR | 1.11 (3H, t), 1.4 (6H, s), 2.33 (3H, s), 2.37 (2H, q), 2.85 (2H, s), 5.49 (2H, s), 6.09 (1H, s), 7.18 (2H, m), 7.37-7.61 (2H, m), 7.76 (1H, d), 8.25 (1H, |
| | (CDCl₃, δ ppm): | d), 8.50 (1H, s). |
| 14-119 | ¹H-NMR | 1.13 (3H, t), 1.39 (6H, s), 2.32 (3H, s), 2.40 (2H, q), 2.85 (2H, s), 5.49 (2H, s), 6.11 (1H, s), 7.16 (2H, m), 7.42-7.56 (2H, m), 7.75 (1H, d), |
| | (CDCl₃, δ ppm): | 8.24 (1H, d), 8.51 (1H, s). |
| 14-120 | ¹H-NMR | 1.10 (3H, t), 1.39 (6H, s), 2.32 (3H, s), 2.40 (2H, q), 2.85 (2H, s), 5.51 (2H, s), 6.08 (1H, s), 7.01-7.19 (2H, m), 7.42-7.57 (2H, m), 7.76 (1H, d), |
| | (CDCl₃, δ ppm): | 8.25 (1H, dz), 8.49 (1H, s). |
| 14-121 | ¹H-NMR | 1.15 (3H, t), 1.57 (6H, d), 2.33 (3H, s), 2.47 (2H, q), 2.95 (2H, dd), 5.47 (2H, s), 6.91 (1H, s), 7.18 (2H, m), 7.40-7.55 (2H, m), 7.69 (1H, d), |
| | (CDCl₃, δ ppm): | 8.17 (1H, d), 8.40 (1H, s). |
| 14-122 | ¹H-NMR | 1.15 (3H, t), 1.58 (6H, s), 2.31 (3H, s), 2.47 (2H, q), 2.95 (2H, dd), 5.48 (2H, s), 6.89 (1H, s), 7.15-7.22 (2H, m), 7.40-7.55 (2H, m), 7.70 (1H, d), |
| | (CDCl₃, δ ppm): | 8.16 (1H, d), 8.39 (1H, s). |
| 14-123 | ¹H-NMR (CDCl₃, δ ppm): | 1.16 (3H, t), 1.56 (6H, s), 2.31 (3H, s), 2.47 (2H, q), 2.94 (2H, dd), 5.49 (2H, s), 6.83 (1H, s), 7.14-7.21 (3H, m), 7.43-7.72 (2H, m), 8.18 (1H, d), 8.37 (1H, s). |
| 14-124 | ¹H-NMR | 1.15 (3H, t), 1.57 (6H, d), 2.31 (3H, s), 2.49 (2H, q), 2.94 (2H, dd), 5.52 (2H, s), 6.83 (1H, s), 7.14-7.20 (2H, m), 7.56 (3H, m), 8.17 (1H, d), |
| | (CDCl₃, δ ppm): | 8.37 (1H, s). |
| 14-125 | ¹H-NMR | 1.20 (3H, t), 1.65 (6H, s), 2.32 (3H, s), 2.70 (2H, m), 3.44 (2H, s), 5.47 (2H, s), 6.42 (1H, s), 7.19 (2H, m), 7.49 (2H, m), 7.69 (1H, d), 8.18 (2H, |
| | (CDCl₃, δ ppm): | m). |
| 14-126 | ¹H-NMR | 1.20 (3H, t), 1.64 (6H, s), 2.32 (3H, s), 2.71 (2H, q), 3.44 (2H, s), 5.50 (2H, s), 6.44 (1H, s), 7.21 (2H, m), 7.41-7.70 (3H, m), 8.18 (2H, s). |
| | (CDCl₃, δ ppm): | |
| 14-127 | ¹H-NMR | 1.23 (3H, t), 1.63 (6H, s), 2.32 (3H, s), 2.71 (2H, q), 3.44 (2H, s), 5.49 (2H, s), 6.42 (1H, s), 7.14-7.26 (3H, m), 7.58 (2H, m), 8.17 (2H, s). |
| | (CDCl₃, δ ppm): | |
| 14-128 | ¹H-NMR | 1.22 (3H, t), 1.62 (6H, s), 2.32 (3H, s), 2.71 (2H, q), 3.44 (2H, s), 5.52 (2H, s), 6.43 (1H, s), 7.15-7.23 (2H, m), 7.38-7.71 (3H, m), 8.18 (2H, m). |
| | (CDCl₃, δ ppm): | |
| 14-130 | ¹H-NMR | 1.40 (6H, s), 2.29 (3H, s), 5.49 (2H, s), 6.63 (1H, s), 7.15-8.24 (7H, m), 9.37 (1H, s) |
| | (CDCl₃, δ ppm): | |
| 14-133 | ¹H-NMR | 1.33 (6H, s), 2.31 (3H, s), 2.43-2.70 (1H, br), 3.35 (2H, s), 4.42 (2H, s), 5.48 (2H, s), 6.21 (1H, s), 7.04-8.57 (12H, m) |
| | (CDCl₃, δ ppm): | |
| 14-135 | ¹H-NMR | 1.04 (3H, t), 1.13 (3H, d), 2.32 (3H, s), 3.06-3.15 (2H, m), 3.93-4.14 (2H, m), 4.25-4.40 (1H, m), 4.57-4.71 (1H, br), 5.49 (2H, s), 6.43-6.61 (1H, br), |
| | (CDCl₃, δ ppm): | 7.26-8.43 (7H, m) |
| 14-136 | ¹H-NMR | 1.05 (3H, t), 1.14 (3H, d), 2.32 (3H, s), 3.04-3.14 (2H, m), 3.93-4.16 (2H, m), 4.29-4.46 (1H, m), 4.61-4.71 (1H, br), 5.51 (2H, s), 6.49 (1H, d), |
| | (CDCl₃, δ ppm): | 7.15-8.41 (7H, m) |
| 14-137 | ¹H-NMR | 1.41 (3H, d), 2.28 (3H, s), 4.68-4.84 (1H, m), 5.09 (2H, s), 5.45 (2H, s), 6.76-8.30 (13H, m) |
| | (CDCl₃, δ ppm): | |
| 14-138 | ¹H-NMR | 1.41 (3H, d), 2.26 (3H, s), 4.68-4.80 (1H, m), 5.10 (2H, s), 5.49 (2H, s), 6.75-8.31 (13H, m) |
| | (CDCl₃, δ ppm): | |
| 14-139 | ¹H-NMR | 1.58 (6H, s), 2.29 (3H, s), 5.07 (2H, s), 5.45 (2H, s), 6.80-8.40 (13H, m) |
| | (CDCl₃, δ ppm): | |
| 14-140 | ¹H-NMR | 1.46 (3H, d), 2.30 (3H, s), 3.68 (3H, s), 3.53-4.07 (2H, m), 4.61-4.77 (1H, m), 5.47 (2H, s), 6.52 (1H, d), 7.13-8.26 (8H, m) |
| | (CDCl₃, δ ppm): | |
| 14-141 | ¹H-NMR | 1.46 (3H, d), 2.29 (3H, s), 3.68 (3H, s), 3.61-4.05 (2H, m), 4.64-4.80 (1H, m), 5.51 (2H, s), 6.54 (1H, d), 7.07-8.32 (8H, m) |
| | (CDCl₃, δ ppm): | |
| 14-142 | ¹H-NMR | 1.62 (6H, s), 2.32 (3H, s), 3.66 (3H, s), 3.72 (2H, d), 5.48 (2H, s), 6.33 (1H, s), 7.06-8.50 (8H, m) |
| | (CDCl₃, δ ppm): | |
| 14-143 | ¹H-NMR (DMSO-d₆, δ ppm): | 0.01-0.13 (4H, m), 1.37 (6H, s), 2.24 (3H, s), 2.50 (1H, br), 5.70 (2H, s), 7.18-7.84 (7H, m), 8.67 (1H, s), 10.29 (1H, s) |
| 14-144 | ¹H-NMR | 1.40 (6H, s), 2.26 (3H, s), 2.81 (1H, s), 3.58 (2H, br), 5.69 (2H, s), 7.16-8.0 (7H, m), 8.83 (1H, s), 10.22 (1H, s) |
| | (CDCl₃, δ ppm): | |
| 14-145 | ¹H-NMR | 1.3 (3H, d) 1.9 (3H, s), 2.4 (3H, s), 2.6-2.7 (2H, m) 4.3-4.4 (1H, m), 6.1 (1H, br d), 7.1 (1H, s), 7.2 (1H, br d), 7.5 (1H, t), 7.6 (1H, d), 7.8 (1H, br |
| | (CDCl₃, δ ppm): | d), 8.6 (1H, br d), 8.7 (1H, br s). |
| 14-146 | ¹H-NMR | 1.21 (3H, d), 1.96 (3H, s), 2.31 (3H, s), 2.55 (1H, dd), 2.60 (1H, dd), 4.26-4.40 (1H, m), 5.41 (2H, s), 6.21 (1H, d), 6.85 (1H, s), 6.89 (1H, d), |
| | (CDCl₃, δ ppm): | 7.45 (1H, dd), 7.55 (1H, d), 7.74 (1H, d), 8.20 (1H, d), 8.44 (1H, bs) |
| 14-147 | ¹H-NMR | 1.4 (3H, d), 2.3 (3H, s), 2.4 (3H, s), 2.9 (2H, m), 4.6 (1H, m), 6.7 (1H, d), 7.2 (1H, s), 7.3 (1H, d), 7.5 (1H, t), 7.6 (1H, d), 7.7 (1H, d), 8.5 (1H, d), |
| | (CDCl₃, δ ppm): | 8.6 (1H, s). |
| 14-148 | ¹H-NMR | 1.2 (3H, d), 1.9 (3H, s), 2.3 (3H, s), 2.5-2.6 (2H, m), 4.3-4.4 (1H, m), 4.3 (2H, s), 6.4 (1H, d), 7.1-8.0 (7H, m), 8.4 (1H, s) |
| | (CDCl₃, δ ppm): | |
| 14-149 | ¹H-NMR | 1.24 (3H, d), 1.31 (3H, t), 1.96 (3H, s), 2.28 (3H, s), 2.51-2.76 (2H, m), 4.08 (2H, q), 4.18 (2H, s), 4.25-4.42 (1H, m), 6.26 (1H, d), 7.00-8.39 (7H, m) |
| | (CDCl₃, δ ppm): | |
| 14-150 | ¹H-NMR | 1.24 (3H, d), 1.92 (3H, s), 2.21 (3H, s), 2.46-2.63 (2H, m), 4.09 (2H, s), 4.24-4.34 (1H, m), 6.76-7.30 (7H, m), 8.59 (1H, s) |
| | (CDCl₃, δ ppm): | |
| 14-151 | ¹H-NMR | 1.22 (3H, d), 1.97 (3H, s), 2.27 (3H, s), 2.50-2.66 (2H, m), 3.77 (3H, s), 4.14 (2H, s), 4.28-4.37 (1H, m), 6.37 (1H, d), 7.01-8.41 (7H, m) |
| | (CDCl₃, δ ppm): | |
| 14-152 | ¹H-NMR | 1.2 (3H, d), 2.0 (3H, s), 2.3 (3H, s), 2.5-2.7 (2H, m), 4.1 (2H, s), 4.3-4.4 (1H, m), 6.4 (1H, d), 7.1-7.9 (7H, m), 8.4 (1H, s) |
| | (CDCl₃, δ ppm): | |
| 14-155 | ¹H-NMR | 1.32 (3H, t), 1.48 (3H, d), 2.27 (3H, s), 2.83 (3H, s), 3.10-3.40 (2H, m), 4.15 (2H, q), 4.18 (2H, q), 4.04-4.18 (1H, m), 6.52 (1H, d), 7.02-7.93 (7H, m) |
| | (CDCl₃, δ ppm): | |
| 14-156 | ¹H-NMR | 1.46 (3H, d), 2.26 (3H, s), 2.74 (3H, s), 2.93-3.19 (2H, m), 4.15 (2H, s), 4.42-4.57 (1H, m), 6.91-8.23 (8H, m) |
| | (CDCl₃, δ ppm): | |
| 14-157 | ¹H-NMR | 1.24 (3H, s), 1.96 (3H, s), 2.30 (3H, s), 4.14 (2H, s), 4.28-4.41 (1H, m), 6.21 (1H, d), 7.15-8.34 (7H, m) |
| | (CDCl₃, δ ppm): | |
| 14-158 | ¹H-NMR | 1.24 (3H, d), 1.96 (3H, s), 2.30 (3H, s), 2.51-2.67 (2H, m), 4.14 (2H, s), 4.19-4.38 (1H, m), 6.24 (1H, d), 7.16-8.34 (7H, m) |
| | (CDCl₃, δ ppm): | |
| 14-159 | ¹H-NMR | 1.24 (3H, d), 1.94 (3H, s), 2.25 (3H, s), 2.33-2.66 (2H, m), 4.13 (2H, s), 4.15-4.37 (1H, m), 6.40 (1H, d), 7.16-8.39 (7H, m) |
| | (CDCl₃, δ ppm): | |
| 14-160 | ¹H-NMR | 1.36 and 1.45 (3H, d and d), 2.27 (6H, br), 2.68-2.93 (2H, m), 4.11 (2H, s), 4.36-4.57 (1H, br), 7.15-8.35 (8H, m) |
| | (CDCl₃, δ ppm): | |
| 14-161 | ¹H-NMR (CDCl₃, δ ppm): | 1.37 and 1.45 (3H, d and d), 2.18-2.39 (6H, m), 2.71-2.89 (2H, m), 4.14 (2H, s), 4.14-4.41 (1H, m), 7.15-8.37 (8H, m) |
| 14-162 | ¹H-NMR | 1.50 (3H, d), 2.29 (3H, s), 2.77 (3H, s), 3.11-3.37 (2H, m), 4.15 (2H, s), 4.57-4.66 (1H, m), 6.60 (1H, d), 7.17-7.97 (7H, m) |
| | (CDCl₃, δ ppm): | |
| 14-165 | ¹H-NMR | 1.32-1.37 (3H, m), 2.36-2.38 (3H, m), 2.70-3.08 (2H, m), 4.41-4.51 (1H, m), 5.79 (2H, s), 7.42 (1H, d), 7.54-7.64 (3H, m), 7.72-7.73 (1H, m), |
| | (CDCl₃, δ ppm): | 8.00 (1H, t), 8.30 (1H, d), 9.09 (1H, s) |
| 14-166 | ¹H-NMR | 1.45 (3H, d), 2.88 (3H, s), 3.10-3.46 (2H, m), 4.50-4.59 (1H, m), 5.49 (2H, s), 6.83 (1H, d), 7.28 (1H, d), 7.29-7.55 (2H, m), 7.59 (1H, d), |
| | (CDCl₃, δ ppm): | 7.61 (1H, d), 8.39 (1H, d), 8.56 (1H, s) |
| 14-167 | ¹H-NMR | 1.31-1.52 (3H, m), 2.38 (3H, s), 2.78-3.03 (2H, m), 4.36-4.54 (1H, m), 5.50 (2H, s), 7.25-7.65 (5H, m), 8.46 (1H, d), 8.70 (1H, bs) |
| | (CDCl₃, δ ppm): | |
| 14-168 | ¹H-NMR | 1.48 (3H, s), 2.90 (3H, s), 3.16 (1H, dd), 3.44 (1H, dd), 4.50-4.64 (1H, m), 5.51 (2H, s), 6.72 (1H, d), 7.24-7.64 (5H, m), 8.43 (1H, t), 8.51 (1H, |
| | (CDCl₃, δ ppm): | bs) |
| 14-169 | ¹H-NMR | 1.25 (3H, d), 1.91 (3H, s), 2.32 (3H, s), 2.48-2.64 (2H, m), 4.23-4.41 (1H, m), 5.49 (2H, s), 6.15 (1H, d), 7.16-8.40 (7H, m) |
| | (CDCl₃, δ ppm): | |
| 14-170 | ¹H-NMR | 1.23 (3H, d), 1.93 (3H, s), 2.32 (3H, s), 2.56 (2H, m), 4.32 (1H, m), 5.50 (2H, s), 6.1 (1H, d), 7.16-7.40 (3H, m), 7.75 (2H, m), 8.16 (1H, d), |
| | (CDCl₃, δ ppm): | 8.41 (1H, s) |
| 14-172 | ¹H-NMR | 1.23 (3H, d), 1.91 (3H, s), 2.30 (3H, s), 2.46-2.66 (2H, m), 4.25-4.41 (1H, m), 5.52 (2H, s), 6.0-6.59 (3H, m), 7.15-8.37 (7H, m) |
| | (CDCl₃, δ ppm): | |
| 14-173 | ¹H-NMR | 1.23 (3H, d), 1.93 (3H, s), 2.30 (3H, s), 2.48-2.64 (2H, m), 4.25-4.41 (1h, m), 5.51 (2H, s), 6.14 (1H, d), 7.14-8.39 (7H, m) |
| | (CDCl₃, δ ppm): | |
| 14-174 | ¹H-NMR | 1.36 (3H, d), 2.27 (3H, s), 2.31 (3H, s), 2.81 (2H, m), 4.42-4.55 (1H, m), 5.48 (2H, d), 7.16-7.38 (4H, m), 7.69 (2H, t), 8.10 (1H, m), 8.39 (1H, s) |
| | (CDCl₃, δ ppm): | |
| 14-175 | ¹H-NMR | 1.49 (3H, d), 2.04 (3H, s), 2.32 (3H, s), 2.80 (2H, s), 4.62 (1H, m), 5.50 (2H, s), 6.62 (1H, m), 7.18-8.01 (7H, m) |
| | (CDCl₃, δ ppm): | |
| 14-176 | ¹H-NMR | 1.10 (3H, t), 1.23 (3H, d), 2.32 (3H, s), 2.41 (2H, m), 2.52-2.74 (2H, m), 4.24-4.38 (1H, m), 5.49 (2H, s), 6.20 (1H, d), 7.19 (2H, m), 7.36 (1H, m), |
| | (CDCl₃, δ ppm): | 7.68-7.79 (2H, m), 8.14 (1H, d), 8.41 (1H, s). |
| 14-177 | ¹H-NMR | 1.12 (3H, t), 1.38 (3H, d), 2.32 (3H, s), 2.49 (2H, m), 2.70-2.80 (2H, m), 4.47-4.56 (1H, m), 5.48 (2H, s), 6.87 (1H, d), 7.17-7.28 (3H, m), |
| | (CDCl₃, δ ppm): | 7.71 (2H, d), 8.09 (1H, m), 8.36 (1H, m). |
| 14-178 | ¹H-NMR | 1.26 (3H, t), 1.49 (3H, d), 2.31 (3H, s), 2.89 (2H, q), 3.19 (2H, ddd), 4.57-4.65 (1H, m), 5.49 (2H, s), 6.65 (1H, dz), 7.19 (2H, d), 7.37 (1H, m), |
| | (CDCl₃, δ ppm): | 7.69-7.74 (2H, m), 8.06 (2H, m). |
| 14-179 | ¹H-NMR | 1.40 (6H, s), 1.95 (3H, s), 2.33 (3H, s), 2.83 (2H, s), 5.49 (2H, s), 6.05 (1H, s), 7.15-7.26 (2H, m), 7.38 (1H, t), 7.76 (2H, m), 8.27 (1H, dz), |
| | (CDCl₃, δ ppm): | 8.45 (1H, s) |
| 14-180 | ¹H-NMR (CDCl₃, δ ppm): | 1.57 (6H, d), 2.26 (3H, s), 2.31 (3H, s), 3.00 (2H, dd), 5.50 (2H, s), 6.73 (1H, s), 7.21 (2H, m), 7.37 (1H, t), 7.70-7.75 (2H, m), 8.21 (1H, d), 8.36 (1H, s). |
| 14-181 | ¹H-NMR | 1.64 (6H, s), 2.33 (3H, s), 2.60 (3H, s), 3.52 (2H, s), 5.49 (2H, s), 6.41 (1H, s), 7.19 (2H, m), 7.46 (1H, m), 7.72 (2H, d), 8.19 (2H, s). |
| | (CDCl₃, δ ppm): | |
| 14-182 | ¹H-NMR | 1.10 (3H, t), 1.40 (6H, s), 2.33 (3H, s), 2.40 (2H, q), 2.83 (2H, s), 5.46 (2H, s), 6.07 (1H, s), 7.22 (2H, m), 7.38 (1H, t), 7.76 (2H, m), 8.27 (1H, d), |
| | (CDCl₃, δ ppm): | 8.47 (1H, s). |
| 14-184 | ¹H-NMR | 1.10 (3H, t), 1.39 (6H, s), 2.32 (3H, s), 2.40 (2H, q), 2.83 (2H, s), 5.48 (2H, s), 6.06 (1H, s), 7.16 (2H, m), 7.38 (1H, m), 7.76 (2H, m), 8.27 (1H, d), |
| | (CDCl₃, δ ppm): | 8.46 (1H, s). |
| 14-185 | ¹H-NMR | 1.10 (3H, t), 1.41 (6H, s), 2.31 (3H, s), 2.39 (2H, q), 2.83 (2H, s), 5.51 (2H, s), 6.07 (1H, s), 7.18 (2H, mz), 7.38 (1H, t), 7.76 (2H, m), 8.27 (1H, d), |
| | (CDCl₃, δ ppm): | 8.46 (1H, s). |
| 14-186 | ¹H-NMR | 1.15 (3H, t), 1.59 (6H, s), 2.31 (3H, s), 2.47 (2H, dq), 2.93 (2H, dd), 5.47 (2H, s), 6.91 (1H, s), 7.18 (2H, mz), 7.36 (1H, t), 7.69-7.74 (2H, m), |
| | (CDCl₃, δ ppm): | 8.19 (1H, d), 8.38 (1H, s). |
| 14-187 | ¹H-NMR | 1.12-1.28 (3H, m), 1.59 (6H, s), 2.31 (3H, s), 2.47 (2H, q), 2.93 (2H, dd), 5.50 (2H, s), 6.93 (1H, s), 7.18 (2H, m), 7.35 (1H, t), 7.69-7.74 (2H, m), |
| | (CDCl₃, δ ppm): | 8.18 (1H, d), 8.39 (1H, s). |
| 14-188 | ¹H-NMR | 1.15 (3H, t), 1.58 (6H, s), 2.31 (3H, s), 2.46 (2H, m), 2.93 (2H, dd), 5.49 (2H, s), 6.92 (1H, s), 7.17 (2H, m), 7.36 (1H, t), 7.69-7.74 (2H, m), |
| | (CDCl₃, δ ppm): | 8.19 (1H, d), 8.38 (1H, s). |
| 14-189 | ¹H-NMR | 1.20 (3H, t), 1.58 (6H, s), 2.31 (3H, s), 2.47 (2H, q), 2.93 (2H, dd), 5.52 (2H, s), 6.92 (1H, s), 7.17 (2H, m), 7.36 (1H, t), 7.72 (2H, m), 8.18 (1H, |
| | (CDCl₃, δ ppm): | d), 8.38 (1H, s). |
| 14-190 | ¹H-NMR | 1.20 (3H, t), 1.64 (6H, s), 2.32 (3H, s), 2.70 (2H, q), 3.42 (2H, s), 5.47 (2H, s), 6.43 (1H, s), 7.16-7.21 (2H, m), 7.38 (1H, t), 7.73 (2H, d), 8.20 (2H, |
| | (CDCl₃, δ ppm): | m). |
| 14-191 | ¹H-NMR | 1.19 (3H, t), 1.64 (6H, s), 2.31 (3H, s), 2.63-2.74 (2H, m), 3.43 (2H, s), 5.50 (2H, s), 6.46 (1H, s), 7.19 (2H, m), 7.38 (1H, t), 7.72 (2H, d), |
| | (CDCl₃, δ ppm): | 8.19 (2H, m). |
| 14-192 | ¹H-NMR | 1.19 (3H, t), 1.64 (6H, s), 2.32 (3H, s), 2.63-2.74 (2H, m), 3.42 (2H, s), 5.49 (2H, s), 6.43 (1H, s), 7.17 (2H, m), 7.38 (1H, t,), 7.73 (2H, d), |
| | (CDCl₃, δ ppm): | 8.20 (2H, m). |
| 14-193 | ¹H-NMR | 1.19 (3H, t), 1.64 (6H, s), 2.32 (3H, s), 2.70 (2H, q), 3.42 (2H, s), 5.52 (2H, s), 6.41 (1H, s), 7.19 (2H, m), 7.38 (1H, t), 7.73 (2H, d), 8.19 (2H, m). |
| | (CDCl₃, δ ppm): | |
| 14-194 | ¹H-NMR | 1.25 (3H, d), 1.95 (3H, s), 2.54 (1H, dd), 2.66 (1H, dd), 4.23-4.37 (1H, m), 5.50 (2H, s), 6.27 (1H, d), 7.13-7.30 (3H, m), 7.60-7.71 (3H, m), |
| | (CDCl₃, δ ppm): | 7.88 (1H, d), 9.03 (1H, bs) |
| 14-197 | ¹H-NMR | 1.3 (3H, d), 2.0 (3H, s), 2.6-2.7 (2H, m), 4.3-4.4 (1H, m), 5.5 (2H, s), 6.1 (1H, d), 7.2-8.5 (6H, m), 8.6 (1H, s) |
| | (CDCl₃, δ ppm): | |
| 14-201 | ¹H-NMR | 1.24 (3H, d), 1.92 (3H, s), 2.31 (3H, s), 2.50-2.64 (2H, m), 4.28-4.33 (1H, m), 5.51 (2H, s), 6.16 (1H, d), 7.14-8.33 (7H, m) |
| | (CDCl₃, δ ppm): | |
| 14-202 | ¹H-NMR (CDCl₃, δ ppm): | 1.39 (3H, d), 2.28 (3H, s), 2.31 (3H, s), 2.84 (2H, m), 4.48 (1H, m), 5.49 (2H, s), 6.95 (1H, d), 7.16-7.26 (3H, m), 7.72 (1H, d), 7.97 (1H, d), 8.21 (2H, m). |
| 14-204 | ¹H-NMR | 1.50 (3H, d), 2.32 (3H, s), 2.80 (3H, s), 3.26 (2H, m), 4.61 (1H, m), 5.50 (2H, s), 6.46 (1H, d), 7.24 (3H, m), 7.74 (1H, d), 7.94-8.11 (3H, m). |
| | (CDCl₃, δ ppm): | |
| 14-205 | ¹H-NMR | 1.50 (3H, d), 2.30 (3H, s), 2.79 (3H, s), 3.07-3.45 (2H, m), 4.54-4.68 (1H, m), 5.51 (2H, s), 6.47 (1H, d), 7.13-8.10 (7H, m) |
| | (CDCl₃, δ ppm): | |
| 14-206 | ¹H-NMR | 1.09 (3H, t), 1.26 (3H, d), 2.32 (3H, s), 2.39 (2H, m), 2.61 (2H, m), 4.30 (1H, m), 5.48 (2H, s), 6.19 (1H, d), 7.21 (3H, m), 7.78 (1H, d), 7.96 (1H, |
| | (CDCl₃, δ ppm): | d), 8.18 (1H, d), 8.35 (1H, s). |
| 14-207 | ¹H-NMR | 1.12 (3H, t), 1.43 (3H, d), 2.31 (3H, s), 2.50 (2H, m), 2.77 (2H, m), 4.52 (1H, m), 5.48 (2H, s), 6.81 (1H, s), 7.16-7.23 (3H, m), 7.73 (1H, d), |
| | (CDCl₃, δ ppm): | 7.95 (1H, m), 8.13 (1H, m), 8.29 (1H, s). |
| 14-208 | ¹H-NMR | 1.25 (3H, t), 1.50 (3H, d), 2.31 (3H, s), 2.90 (2H, q), 3.20 (2H, ddd), 4.61 (1H, m), 5.50 (2H, s), 6.51 (1H, d), 7.24 (3H, dm), 7.74 (1H, d), |
| | (CDCl₃, δ ppm): | 7.99 (2H, m), 8.10 (1H, d). |
| 14-211 | ¹H-NMR | 1.40 (6H, s), 1.92 (3H, s), 2.32 (3H, s), 2.81 (2H, s), 5.51 (2H, s), 6.04 (1H, s), 7.11-8.37 (7H, m) |
| | (CDCl₃, δ ppm): | |
| 14-213 | ¹H-NMR | 1.59 (6H, d), 2.27 (3H, s), 2.31 (3H, s), 2.98 (2H, dd), 5.50 (2H, s), 6.70 (1H, s), 7.15-7.26 (3H, m), 7.76 (1H, d), 7.97 (1H, d), 8.26 (2H, m). |
| | (CDCl₃, δ ppm): | |
| 14-215 | ¹H-NMR | 1.64 (6H, s), 2.34 (3H, s), 2.61 (3H, s), 3.50 (2H, s), 5.48 (2H, s), 6.35 (1H, s), 7.15-7.26 (3H, m), 7.74 (1H, d), 7.98 (1H, d), 8.18 (2H, m). |
| | (CDCl₃, δ ppm): | |
| 14-216 | ¹H-NMR | 1.11 (3H, t), 1.40 (6H, s), 2.32 (3H, s), 2.39 (2H, q), 2.81 (2H, s), 5.46 (2H, s), 6.05 (1H, s), 7.14-7.22 (3H, m), 7.81 (1H, m), 7.98 (1H, m), |
| | (CDCl₃, δ ppm): | 8.34 (2H, m). |
| 14-217 | ¹H-NMR | 1.11 (3H, t), 1.40 (6H, s), 2.32 (3H, s), 2.42 (2H, q), 2.81 (2H, s), 5.48 (2H, s), 6.07 (1H, s), 7.14-7.26 (3H, m), 7.80 (1H, d), 7.96-8.03 (1H, m), |
| | (CDCl₃, δ ppm): | 8.29 (1H, d), 8.40 (1H, s). |
| 14-218 | ¹H-NMR | 1.11 (3H, t), 1.40 (6H, s), 2.30 (3H, s), 2.39 (2H, q), 2.81 (2H, s), 5.48 (2H, s), 6.06 (1H, s), 7.13-7.24 (3H, m), 7.80 (1H, d), 7.98 (1H, d), |
| | (CDCl₃, δ ppm): | 8.30 (1H, d), 8.39 (1H, s). |
| 14-219 | ¹H-NMR | 1.11 (3H, t), 1.40 (6H, s), 2.32 (3H, s), 2.42 (2H, q), 2.81 (2H, s), 5.51 (2H, s), 6.06 (1H, s), 7.13-7.30 (3H, m), 7.80 (1H, d), 7.97 (1H, d), |
| | (CDCl₃, δ ppm): | 8.31 (2H, m). |
| 14-220 | ¹H-NMR | 1.17 (3H, t), 1.59 (6H, d), 2.31 (3H, s), 2.48 (2H, q), 2.91 (2H, dd), 5.47 (2H, s), 6.89 (1H, s), 7.14-7.26 (3H, m), 7.75 (1H, d), 7.96 (1H, d), |
| | (CDCl₃, δ ppm): | 8.26 (2H, m). |
| 14-221 | ¹H-NMR | 1.16 (3H, t), 1.61 (6H, d), 2.31 (3H, s), 2.49 (2H, q), 2.92 (2H, dd), 5.49 (2H, s), 6.87 (1H, s), 7.14-7.21 (3H, m), 7.75 (1H, d), 7.96 (1H, d), |
| | (CDCl₃, δ ppm): | 8.26 (2H, m). |
| 14-222 | ¹H-NMR | 1.17 (3H, t), 1.59 (6H, d), 2.31 (3H, s), 2.49 (2H, q), 2.90 (2H, dd), 5.49 (2H, s), 6.87 (1H, s), 7.13-7.26 (3H, m), 7.75 (1H, d), 7.96 (1H, d), |
| | (CDCl₃, δ ppm): | 8.26 (2H, m). |
| 14-223 | ¹H-NMR | 1.16 (3H, t), 1.61 (6H, s), 2.31 (3H, s), 2.49 (2H, q), 2.90 (2H, dd), 5.52 (2H, s), 6.86 (1H, s), 7.13-7.26 (3H, m), 7.76 (1H, d), 7.96 (1H, d), |
| | (CDCl₃, δ ppm): | 8.25 (2H, m). |
| 14-224 | ¹H-NMR | 1.20 (3H, t), 1.65 (6H, s), 2.32 (3H, s), 2.63-2.75 (2H, m), 3.42 (2H, s), 5.47 (2H, s), 6.45 (1H, s), 7.15-7.26 (3H, m), 7.74 (1H, d), 7.89-8.00 (1H, |
| | (CDCl₃, δ ppm): | m), 8.20 (2H, m). |
| 14-225 | ¹H-NMR (CDCl₃, δ ppm): | 1.20 (3H, t), 1.65 (6H, s), 2.32 (3H, s), 2.71 (2H, q), 3.41 (2H, s), 5.49 (2H, s), 6.40 (1H, s), 7.15-7.26 (3H, m), 7.75 (1H, d), 7.98 (1H, d), 8.19 (2H, m). |
| 14-226 | ¹H-NMR | 1.23 (3H, t), 1.65 (6H, s), 2.32 (3H, s), 2.64-2.76 (2H, m), 3.43 (2H, s), 5.49 (2H, s), 6.51 (1H, s), 7.14-7.26 (3H, m), 7.93-8.04 (2H, m), 8.22 (2H, |
| | (CDCl₃, δ ppm): | m). |
| 14-227 | ¹H-NMR | 1.20 (3H, t), 1.65 (6H, s), 2.32 (3H, s), 2.72 (2H, q), 3.43 (2H, s), 5.52 (2H, s), 6.54 (1H, s), 7.14-7.26 (3H, m), 7.99 (2H, m), 8.22 (2H, m). |
| | (CDCl₃, δ ppm): | |
| 14-232 | ¹H-NMR | 1.2 (3H, d), 1.9 (3H, s), 2.4 (3H, s), 2.5-2.6 (2H, m), 4.3-4.4 (1H, m), 6.1 (1H, br d), 7.1 (1H, s), 7.2-7.3 (2H, m), 7.8 (1H, m), 8.0 (1H, d), |
| | (CDCl₃, δ ppm): | 8.6 (1H, s), 8.6 (1H, d). |
| 14-233 | ¹H-NMR | 1.2 (3H, d), 1.9 (3H, s), 2.4 (3H, s), 2.6 (2H, m), 4.3-4.4 (1H, m), 6.1 (1H, d), 7.2 (1H, s), 7.2-7.3 (2H, m), 7.8 (1H, d), 8.0 (1H, d), 8.6 (1H, s), |
| | (CDCl₃, δ ppm): | 8.6 (1H, d). |
| 14-234 | ¹H-NMR | 1.4 (3H, m), 2.3 (3H, m), 2.4 (3H, m), 2.8-2.9 (2H, m), 4.4 (1H, m), 4.5 (1H, m), 7.2 (1H, m), 7.2-7.3 (2H, m), 7.5 (1H, d), 7.8 (1H, d), 8.0 (1H, |
| | (CDCl₃, δ ppm): | d), 8.5-8.6 (2H, m). |
| 14-235 | ¹H-NMR | 1.4 (3H, m), 2.3 (3H, m), 2.4 (3H, m), 2.8-2.9 (2H, m), 4.4-4.5 (1H, m), 7.1-7.4 (3H, m), 7.8 (1H, d), 8.0 (1H, d), 8.5 (1H, s), 8.5 (1H, d). |
| | (CDCl₃, δ ppm): | |
| 14-265 | ¹H-NMR | 1.20 (3H, d), 1.92 (3H, s), 2.32 (3H, s), 2.50-2.64 (2H, m), 4.15-4.38 (1H, m), 5.50 (2H, s), 6.23 (1H, d), 7.16 (1H, s), 7.19 (1H, d), 7.71 (1H, t), |
| | (CDCl₃, δ ppm): | 8.12 (1H, d), 8.23 (1H, d), 8.30 (1H, d), 8.40 (1H, d) |
| 14-267 | ¹H-NMR | 1.27 (3H, d), 2.03 (3H, s), 2.33 (3H, s), 2.60 (1H, dd), 2.65 (1H, dd), 4.25-4.38 (1H, m), 5.47 (2H, s), 6.32 (1H, d), 7.14-7.24 (2H, m), |
| | (CDCl₃, δ ppm): | 7.47-7.56 (2H, m), 7.82 (1H, d), 8.07 (1H, d), 8.88 (1H, bs) |
| 14-292 | ¹H-NMR | 1.23 (3H, d), 1.93 (3H, s), 2.32 (3H, s), 2.45-2.67 (2H, m), 4.22-4.38 (1H, m), 5.34 (2H, s), 6.25 (1H, d), 7.07-7.29 (3H, m), 7.76 (1H, d), |
| | (CDCl₃, δ ppm): | 7.96 (1H, d), 8.09 (1H, s), 8.20 (1H, d), 8.38 (1H, s) |
| 14-293 | ¹H-NMR | 1.25 (3H, d), 1.94 (3H, s), 2.33 (3H, s), 2.49-2.65 (2H, m), 4.22-4.38 (1H, m), 5.32 (2H, s), 6.29 (1H, d), 7.09-7.22 (3H, m), 7.77 (1H, d), |
| | (CDCl₃, δ ppm): | 7.95 (1H, d), 8.08 (1H, s), 8.19 (1H, d), 8.39 (1H, s) |
| 14-294 | ¹H-NMR | 1.25 (3H, d), 1.96 (3H, s), 2.33 (3H, s), 2.49-2.67 (2H, m), 5.32 (2H, s), 6.28 (1H, d), 7.10-7.62 (4H, m), 7.74 (1H, d), 8.09 (1H, s), 8.16 (1H, d), |
| | (CDCl₃, δ ppm): | 8.49 (1H, s) |
| 14-299 | ¹H-NMR | 1.18 (3H, d), 1.91 (3H, s), 2.28 (3H, s), 2.45 (1H, dd), 2.63 (1H, dd), 4.08-4.15 (1H, m), 5.84 (2H, s), 7.27 (1H, d), 7.31 (1H, s), 7.39 (1H, dd), |
| | (CD₃CN, δ ppm): | 7.67-7.76 (2H, m), 7.82 (2H, d), 8.25 (2H, d), 8.82 (1H, bs) |
| 14-301 | ¹H-NMR | 2.35 (3H, s), 2.76-2.86 (3H, m), 5.72 (2H, s), 7.24-7.31 (2H, m), 7.75-7.80 (3H, m), 8.07 (1H, d), 8.19 (1H, d), 9.02 (1H, s) |
| | (Acetone-d₆, δ ppm): | |
| 14-303 | ¹H-NMR | 1.18 (3H, d), 1.95 (3H, s), 2.32 (3H, s), 2.52-2.57 (2H, m), 4.29-4.34 (1H, m), 5.43 (1H, d), 5.47 (2H, s), 5.82 (1H, d), 6.07 (1H, d), 6.88 (1H, dd), |
| | (CDCl₃, δ ppm): | 7.16-7.20 (2H, m), 7.48 (1H, t), 7.69-7.74 (2H, m), 8.22-8.27 (2H, m) |
| 14-304 | ¹H-NMR | 1.25 (3H, d), 1.97 (3H, s), 2.50-2.67 (2H, m), 4.07 (2H, s), 4.31-4.40 (1H, m), 4.85 (2H, q), 6.16 (1H, d), 7.25-7.29 (2H, m), 7.40-7.58 (4H, m), |
| | (CDCl₃, δ ppm): | 7.72 (1H, d), 8.70 (1H, s) |
| 14-308 | ¹H-NMR (CD₃CN, δ ppm): | 1.20 (3H, d), 1.92 (3H, s), 2.28 (3H, s), 2.51 (1H, dd), 2.59 (1H, dd), 4.13-4.23 (1H, m), 5.57 (2H, s), 7.09 (1H, d), 7.18 (1H, d), 7.21 (1H, s), 7.75 (1H, d), 8.53 (1H, bs), 8.75 |
| | | (1H, s), 8.84 (1H, s) |
| 14-312 | ¹H-NMR | 1.23 (3H, s), 1.94 (3H, s), 2.34 (3H, s), 2.51-2.64 (2H, m), 4.33 (1H, m), 5.46 (2H, s), 6.03-6.41 (2H, m), 7.18 (2H, dd), 7.43-7.57 (2H, m), |
| | (CDCl₃, δ ppm): | 7.75 (1H, d), 8.15 (1H, d), 8.41 (1H, s). |
| 14-313 | ¹H-NMR | 1.25 (3H, d), 1.94 (3H, s), 2.32 (3H, s), 2.51-2.64 (2H, m), 4.33 (1H, m), 5.49 (2H, d), 6.16-6.58 (2H, m), 7.19 (2H, dd), 7.43-7.57 (2H, m), |
| | (CDCl₃, δ ppm): | 7.74 (1H, d), 8.15 (1H, dd), 8.41 (1H, s). |
| 14-314 | ¹H-NMR | 1.27 (3H, d), 1.95 (3H, s), 2.36 (3H, s), 2.58-2.62 (2H, m), 4.26-4.40 (1H, m), 6.21-6.24 (1H, m), 7.14 (1H, s), 7.40-7.55 (3H, m), 7.76 (1H, |
| | (CDCl₃, δ ppm): | d), 8.13 (1H, s), 8.25 (1H, s), 8.37 (1H, d), 8.63 (1H, br s) |
| 14-315 | ¹H-NMR | 1.31 (3H, d), 2.04 (3H, s), 2.62-2.77 (2H, m), 4.34-4.36 (1H, m), 5.91-5.93 (1H, m), 6.36-6.40 (1H, m), 7.27-7.68 (7H, m), 8.04 (1H, s), |
| | (CDCl₃, δ ppm): | 8.23 (1H, s), 9.67-9.81 (1H, m) |
| 14-316 | ¹H-NMR | 1.24 (3H, d), 1.96 (3H, s), 2.30 (3H, s), 2.46 (3H, s), 2.52-2.65 (2H, m), 4.29-4.38 (1H, m), 5.30 (2H, s), 6.18 (1H, d), 7.03-7.07 (2H, m), 7.46 (1H, |
| | (CDCl₃, δ ppm): | t), 7.55 (1H, d), 7.75 (1H, d), 8.11 (1H, d), 8.40 (1H, s) |
| 14-337 | ¹H-NMR | 1.27 (3H, d), 2.00 (3H, s), 2.56-2.70 (2H, m), 4.33-4.42 (1H, m), 5.49 (2H, s), 6.15 (1H, d), 7.10-7.15 (2H, m), 7.45 (1H, t), 7.56 (1H, d), 7.72 (1H, |
| | (CDCl₃, δ ppm): | d), 8.44 (1H, t), 8.76 (1H, s) |
| 14-338 | ¹H-NMR | 1.32 (3H, d), 2.06 (3H, s), 2.61-2.78 (2H, m), 4.33-4.42 (1H, m), 5.53 (2H, s), 6.20 (1H, d), 7.08 (1H, t), 7.24-7.36 (3H, m), 7.53-7.64 (2H, m), |
| | (CDCl₃, δ ppm): | 9.77 (1H, s) |
| 14-339 | ¹H-NMR | 1.31 (3H, d), 2.06 (3H, s), 2.58-2.77 (2H, m), 4.32-4.41 (1H, m), 5.58 (2H, s), 6.21-6.59 (2H, m), 7.03-7.09 (1H, m), 7.23-7.26 (1H, m), |
| | (CDCl₃, δ ppm): | 7.35-7.36 (2H, m), 7.53-7.64 (2H, m), 9.73 (1H, s) |
| 14-340 | ¹H-NMR | 1.31 (3H, d), 2.04 (3H, s), 2.58-2.76 (2H, m), 4.33-4.42 (1H, m), 5.54 (2H, s), 6.03-6.39 (2H, m), 7.06-7.11 (1H, m), 7.23-7.27 (1H, m), |
| | (CDCl₃, δ ppm): | 7.35-7.41 (2H, m), 7.59-7.66 (2H, m), 9.61 (1H, s) |
| 14-341 | ¹H-NMR | 1.19 (3H, d), 1.99 (3H, s), 2.06 (3H, s), 2.32 (3H, s), 2.54-2.57 (2H, m), 4.19-4.28 (1H, m), 5.47 (2H, s), 6.33 (1H, d), 7.17-7.22 (2H, m), |
| | (CDCl₃, δ ppm): | 7.36-7.49 (2H, m), 7.90-7.98 (2H, m), 8.10-8.12 (1H, m) |
| 14-342 | ¹H-NMR | 1.20 (3H, d), 2.04 (3H, s), 2.27 (3H, s), 2.55-2.65 (2H, m), 4.21-4.32 (1H, m), 5.42 (1H, d), 5.47 (2H, s), 5.81 (1H, d), 6.34 (1H, d), 6.90-6.99 (1H, |
| | (CDCl₃, δ ppm): | m), 7.18-7.26 (2H, m), 7.37-7.55 (3H, m), 7.71 (1H, d), 8.19 (1H, d) |
| 14-343 | ¹H-NMR | 1.35 (3H, m), 2.26 (3H, s), 2.30 (3H, s), 2.75-2.87 (2H, m), 4.48 (1H, m), 5.48 (2H, s), 6.22 (1H, m), 7.18-7.68 (6H, m), 8.07 (1H, t), |
| | (CDCl₃, δ ppm): | 8.40 (1H, d). |
| 14-344 | ¹H-NMR | 1.40 (3H, m), 2.24 (3H, s), 2.30 (3H, s), 2.83 (2H, m), 4.44 (1H, m), 5.49 (2H, d), 6.40 (1H, m), 7.17-7.64 (9H, m), 8.09 (1H, d), 8.43 (1H, |
| | (CDCl₃, δ ppm): | s). |
| 14-345 | ¹H-NMR | 0.07-1.14 (3H, m), 2.21-2.26 (3H, m), 2.50-2.78 (2H, m), 4.24-4.29 (1H, m), 5.53 (2H, s), 6.99-7.31 (5H, m), 7.62-7.70 (2H, m), 8.08-8.28 (1H, |
| | (CDCl₃, δ ppm): | m), 9.91-10.07 (1H, m) |
| 14-346 | ¹H-NMR | 1.05-1.17 (3H, m), 2.22-2.26 (3H, m), 2.50-2.78 (2H, m), 4.22-4.29 (1H, m), 5.50 (2H, s), 6.99-7.31 (5H, m), 7.62-7.70 (2H, m), 8.03-8.24 (1H, |
| | (CDCl₃, δ ppm): | m), 9.88-10.04 (1H, m) |
| 14-347 | ¹H-NMR (CDCl₃, δ ppm): | 1.34-1.44 (3H, m), 2.32 (3H, s), 2.39-2.41 (3H, m), 2.72-2.93 (2H, m), 4.49-4.75 (1H, m), 5.51 (2H, s), 7.15-7.29 (2H, m), 7.65-7.74 (1H, m), 8.18-8.45 (4H, m) |
| 14-351 | ¹H-NMR | 1.45 (3H, s), 2.30 (3H, s), 2.73 (3H, s), 3.23 (2H, m), 4.55-4.64 (1H, m), 5.46 (2H, s), 6.22 (1H, m), 6.69 (1H, d), 7.17 (2H, d), 7.53 (3H, m), |
| | (CDCl₃, δ ppm): | 8.00 (1H, d), 8.11 (1H, s). |
| 14-352 | ¹H-NMR | 1.45 (3H, d), 2.31 (3H, s), 2.74 (3H, s), 3.10-3.37 (2H, m), 4.55-4.64 (1H, m), 5.49 (2H, d), 6.40 (1H, m), 6.71 (1H, d), 7.19 (2H, t), |
| | (CDCl₃, δ ppm): | 7.40-7.64 (3H, m), 7.99 (1H, d), 8.12 (1H, s). |
| 14-354 | ¹H-NMR | 1.23 (3H, d), 2.70 (3H, s), 2.92-3.27 (2H, m), 4.35-4.51 (1H, m), 5.53 (2H, s), 6.96-7.03 (2H, m), 7.18-7.27 (3H, m), 7.59-7.62 (2H, m), 7.93 (1H, |
| | (CDCl₃, δ ppm): | s), 9.81 (1H, s) |
| 14-355 | ¹H-NMR | 1.24 (3H, d), 2.70 (3H, s), 2.92-3.26 (2H, m), 4.41-4.46 (1H, m), 5.51 (2H, s), 6.95-7.02 (2H, m), 7.18-7.21 (3H, m), 7.59-7.62 (2H, m), 7.94 (1H, |
| | (CDCl₃, δ ppm): | m), 9.83 (1H, s) |
| 14-356 | ¹H-NMR | 1.42 (3H, d), 2.31 (3H, s), 2.84 (3H, s), 3.19-3.36 (2H, m), 4.52-4.65 (1H, m), 5.50 (2H, s), 7.17-7.21 (2H, m), 7.70 (1H, t), 8.04-8.29 (3H, m) |
| | (CDCl₃, δ ppm): | |
| 14-373 | ¹H-NMR | diastereomeric mixture1.15-1.50 (3H, m), 2.06-2.22 (3H, m), 2.30-2.35 (3H, m), 2.67-2.89 (5H, m), 4.22-4.61 (1H, m), 5.49 (2H, s), |
| | (CDCl₃, δ ppm): | 7.14-7.25 (2H, m), 7.60-8.11 (5H, m), 8.56-8.72 (1H, m) |
| 14-374 | ¹H-NMR | 1.1 (3H, t), 2.3 (3H, s), 2.3 (3H, s), 2.6-2.9 (2H, m), 3.3 (3H, s), 4.2-4.3 (1H, m), 5.7 (2H, s), 7.1 (1H, d), 7.2-8.8 (6H, m), 9.6 (1H, s) |
| | (CDCl₃, δ ppm): | |
| 14-375 | ¹H-NMR | 1.4 (3H, d), 2.3 (3H, s), 2.8 (3H, s), 3.2-3.4 (2H, m), 3.3 (3H, s), 4.6 (1H, m), 5.5 (2H, s), 6.9 (1H, d), 7.2-8.2 (7H, m) |
| | (CDCl₃, δ ppm): | |
| 14-376 | ¹H-NMR | 1.2 (3H, d), 1.9 (3H, s), 2.3 (3H, s), 2.5 (3H, s), 2.5-2.7 (2H, m), 4.3-4.4 (1H, m), 5.5 (2H, d), 6.4 (1H, bd), 7.2-8.1 (6H, m), 8.4 (1H, s) |
| | (CDCl₃, δ ppm): | |
| 14-380 | ¹H-NMR | 1.2 (3H, d), 1.9 (3H, s), 2.3 (3H, s), 2.5 (3H, s), 2.5-2.6 (2H, m), 4.3 (1H, m), 5.5 (2H, s), 6.4 (1H, bd), 7.1-8.2 (6H, m), 8.4 (1H, s) |
| | (CDCl₃, δ ppm): | |
| 14-382 | ¹H-NMR | 1.2 (3H, t), 1.3 (3H, d), 1.9 (3H, s), 2.3 (3H, s), 2.5-2.6 (2H, m), 3.0 (2H, q), 4.3 (1H, m), 5.5 (2H, s), 6.2 (1H, d), 7.2-8.2 (6H, m), 8.4 (1H, s) |
| | (CDCl₃, δ ppm): | |
| 14-383 | ¹H-NMR | 1.2 (3H, d), 1.9 (3H, s), 2.5 (3H, s), 2.5 (3H, s), 2.5-2.6 (2H, m), 4.3 (1H, m), 5.5 (2H, s), 6.0-8.1 (9H, m), 8.4 (1H, s) |
| | (CDCl₃, δ ppm): | |
| 14-385 | ¹H-NMR | 1.2 (3H, s), 2.0 (3H, s), 2.3 (3H, s), 2.5 (3H, s), 2.5-2.7 (2H, m), 4.1 (2H, s), 4.3-4.4 (1H, m), 6.2 (1H, d), 7.1-8.0 (7H, m), 8.2 (1H, s) |
| | (CDCl₃, δ ppm): | |
| 14-386 | ¹H-NMR | 1.2 (3H, d), 2.0 (3H, s), 2.3 (3H, s), 2.5 (3H, s), 2.5-2.7 (2H, m), 4.3 (2H, s), 4.3-4.4 (1H, m), 6.2 (1H, d), 7.1-8.1 (7H, m), 8.3 (1H, s) |
| | (CDCl₃, δ ppm): | |
| 14-388 | ¹H-NMR | 1.20 (3H, d), 1.91 (3H, s), 2.29 (3H, s), 2.42-2.66 (2H, m), 3.29 (3H, s), 4.11 (2H, s), 4.21-4.33 (1H, m), 6.40 (1H, d), 7.13-7.52 (3H, m), 7.67 (1H, |
| | (CDCl₃, δ ppm): | t), 7.72 (1H, bs), 7.89 (1H, d), 8.00-8.21 (2H, m) |
| 14-392 | ¹H-NMR (CDCl₃, δ ppm): | 1.28 (3H, m), 2.23 (3H, d), 2.82-2.91 (2H, m), 3.30 (3H, d), 4.39-4.63 (1H, m), 5.49 (2H, s), 7.09 (1H, d), 7.31-7.42 (2H, m), 7.69-7.74 (1H, m), 7.95 (1H, d), 8.16-8.21 |
| | | (1H, m), 8.46 (1H, s), 8.52-8.59 (1H, m) |
| 14-399 | ¹H-NMR | 1.19 (3H, d), 2.03 (3H, s), 2.31 (3H, s), 2.52-2.63 (2H, m), 3.48-3.59 (2H, m), 4.24-4.29 (1H, m), 5.48 (2H, s), 6.30 (1H, d), 7.20-7.23 (2H, m), |
| | (CDCl₃, δ ppm): | 7.45-7.52 (2H, m), 7.65 (1H, d), 7.76 (1H, d), 8.12 (1H, d) |
| 14-400 | ¹H-NMR | 1.18 (3H, d), 2.01 (3H, s), 2.30 (3H, s), 2.51-2.59 (2H, m), 4.21-4.26 (1H, m), 5.49 (2H, s), 6.45 (1H, d), 7.20-7.24 (2H, m), 7.53 (1H, t), |
| | (CDCl₃, δ ppm): | 7.70-7.74 (2H, m), 7.85 (1H, d), 8.07 (1H, d) |
| 14-401 | ¹H-NMR | 1.25 (3H, d), 2.00 (3H, s), 2.49-2.69 (5H, m), 4.31-4.40 (1H, m), 5.47 (2H, s), 6.05 (1H, d), 7.28-7.29 (1H, m), 7.38-7.61 (4H, m), 8.54 (1H, d), |
| | (CDCl₃, δ ppm): | 8.62 (1H, s) |
| 14-403 | ¹H-NMR | 0.24-0.58 (2H, m), 1.05 (1H, m), 1.23 (3H, d), 1.93 (3H, s), 2.32 (3H, s), 2.52 (1H, dd), 2.61 (1H, dd), 2.90 (2H, d), 4.33 (1H, m), 5.47 (2H, s), |
| | (CDCl₃, δ ppm): | 6.22 (1H, d), 7.15 (1H, s), 7.18 (1H, d), 7.35-7.68 (3H, m), 8.17 (1H, d), 8.41 (1H, bs) |
| 14-404 | ¹H-NMR | 1.03 (3H, t), 1.22 (3H, d), 1.63-1.77 (2H, m), 1.93 (3H, s), 2.32 (3H, s), 2.49-2.64 (2H, m), 2.94 (2H, t), 4.28-4.37 (1H, m), 5.46 (2H, s), 6.09 (1H, |
| | (CDCl₃, δ ppm): | d), 7.15-7.20 (2H, m), 7.44 (1H, t), 7.52 (1H, d), 7.64 (1H, d), 8.21 (1H, d), 8.36 (1H, s) |
| 14-406 | ¹H-NMR | 1.05 (6H, d), 1.22 (3H, d), 1.85-1.96 (4H, m), 2.32 (3H, s), 2.48-2.64 (2H, m), 2.85 (2H, d), 4.30-4.35 (1H, m), 5.46 (2H, s), 6.13 (1H, d), |
| | (CDCl₃, δ ppm): | 7.15-7.20 (2H, m), 7.43 (1H, t), 7.50 (1H, d), 7.62 (1H, d), 8.20 (1H, d), 8.36 (1H, s) |
| 14-407 | ¹H-NMR | 1.22 (3H, d), 1.89 (3H, s), 2.32 (3H, s), 2.45-2.63 (2H, m), 4.29-4.38 (1H, m), 5.47 (2H, s), 6.23 (1H, d), 7.16-7.21 (2H, m), 7.59 (1H, t), |
| | (CDCl₃, δ ppm): | 7.88-7.95 (2H, m), 8.21 (1H, d), 8.34 (1H, s) |
| 14-409 | ¹H-NMR | 1.21 (3H, d), 1.91 (3H, s), 2.32 (3H, s), 2.47-2.62 (2H, m), 3.54 (2H, q), 4.28-4.33 (1H, m), 5.47 (2H, s), 6.11 (1H, d), 7.16-7.21 (2H, m), |
| | (CDCl₃, δ ppm): | 7.51 (1H, t), 7.75-7.82 (2H, m), 8.22 (1H, d), 8.25 (1H, s) |
| 14-410 | ¹H-NMR | 1.14 (3H, d), 1.88 (3H, s), 2.32 (3H, s), 2.38-2.51 (2H, m), 4.11-4.23 (3H, m), 5.46 (2H, s), 5.71 (1H, d), 7.15-7.25 (4H, m), 7.26-7.31 (3H, m), |
| | (CDCl₃, δ ppm): | 7.41 (1H, t), 7.53 (1H, d), 7.70 (1H, d), 8.20 (1H, d), 8.39 (1H, s) |
| 14-411 | ¹H-NMR | 1.19 (3H, d), 1.93 (3H, s), 2.45-2.65 (2H, m), 3.32 (3H, s), 4.24-4.33 (1H, m), 5.49 (2H, s), 6.28 (1H, d), 7.31 (1H, d), 7.41 (1H, s), 7.28 (1H, t), |
| | (CDCl₃, δ ppm): | 8.00 (1H, d), 8.24 (1H, d), 8.46 (1H, bs), 8.61 (1H, d) |
| 14-413 | ¹H-NMR | 1.38 (3H, d), 2.92 (3H, s), 3.29-3.42 (5H, m), 4.54-4.64 (1H, m), 5.49 (2H, s), 6.74 (1H, d), 7.32 (1H, d), 7.43 (1H, s), 7.45 (1H, t), 7.99 (1H, d), |
| | (CDCl₃, δ ppm): | 8.23 (1H, d), 8.32 (1H, s), 8.54 (1H, d) |
| 14-416 | ¹H-NMR | 1.23 (3H, d), 1.94 (3H, s), 2.32 (3H, s), 2.49-2.65 (2H, m), 4.28-4.37 (1H, m), 5.02 (1H, d), 5.46 (2H, s), 5.94 (1H, t), 6.16 (1H, d), 7.16-7.20 (2H, |
| | (CDCl₃, δ ppm): | m), 7.45-7.70 (3H, m), 8.17-8.20 (1H, m), 8.39 (1H, d) |
| 14-424 | ¹H-NMR | 1.18 (3H, d), 1.88 (3H, s), 2.27 (3H, s), 2.46 (1H, dd), 2.57 (1H, dd), 4.10-4.19 (1H, m), 5.42 (2H, s), 6.98 (1H, d), 7.17 (1H, d), 7.20 (1H, s), |
| | (CD₃CN, δ ppm): | 7.50 (1H, dd), 7.60 (1H, d), 7.70 (1H, d), 7.80 (1H, d), 8.45 (1H, bs) |
| 14-425 | ¹H-NMR | 1.32 (3H, d), 2.27 (3H, s), 2.32 (3H, s), 2.67-2.80 (2H, m), 4.35-4.52 (1H, m), 5.42 (2H, s), 7.02 (1H, d), 7.16 (1H, d), 7.21 (1H, s), 7.46-7.71 (3H, |
| | (CD₃CN, δ ppm): | m), 7.75 (1H, d), 8.33 (1H, bs) |
| 14-436 | ¹H-NMR | 1.53 (6H, s), 2.12 (3H, s), 2.37 (3H, s), 3.05 (2H, s), 5.47 (2H, s), 7.14-7.27 (3H, br), 7.48-7.60 (1H, m), 7.98-8.46 (3H, m), 8.64 (1H, d) |
| | (CDCl₃, δ ppm): | |
| 14-445 | ¹H-NMR (CDCl₃, δ ppm): | 1.2 (3H, d), 2.0 (3H, s), 2.6-2.7 (2H, m), 3.8 (3H, s), 4.3 (1H, m), 5.2 (2H, s), 5.9 (1H, m), 7.1-7.9 (9H, m), 10.9 (1H, s) |
| 14-446 | ¹H-NMR | 1.5 (3H, d), 3.0 (3H, s), 3.2-3.8 (2H, m), 3.9 (3H, s), 4.6 (1H, m), 5.2 (2H, s), 6.2 (1H, d), 7.1-7.9 (9H, m), 12.4 (1H, s) |
| | (CDCl₃, δ ppm): | |
| 14-448 | ¹H-NMR | 1.2 (6H, d), 1.3 (3H, d), 2.1 (3H, s), 2.5-3.0 (2H, m), 4.0-4.5 (2H, m), 5.1 (2H, s), 5.9 (1H, d), 6.3 (1H, d), 7.0-8.0 (6H, m), 8.5-8.7 (3H, m), |
| | (CDCl₃, δ ppm): | 12.0 (1H, s) |
| 15-1 | ¹H-NMR | 1.24 (3H, d), 1.95 (3H, s), 2.28 (3H, s), 2.49-2.66 (2H, m), 4.18 (2H, s), 4.23-4.34 (1H, m), 4.82 (2H, q), 6.25-6.34 (1H, br), 7.04-8.32 (8H, m) |
| | (CDCl₃, δ ppm): | |
| 15-2 | ¹H-NMR | 1.24 (3H, d), 1.95 (3H, s), 2.29 (3H, s), 2.50-2.66 (2H, m), 4.31-4.36 (1H, m), 4.39 (2H, s), 6.22 (1H, d), 7.25-8.29 (8H, m) |
| | (CDCl₃, δ ppm): | |
| 15-3 | ¹H-NMR | 1.24 (3H, d), 1.94 (3H, s), 2.33 (3H, s), 2.51-2.68 (2H, m), 4.28-4.37 (1H, m), 5.55 (1H, s), 6.19 (1H, d), 7.37-8.44 (8H, m) |
| | (CDCl₃, δ ppm): | |
| 15-4 | ¹H-NMR | 1.25 (3H, d), 1.79 (3H, s), 2.31 (3H, s), 2.51-2.66 (2H, m), 3.76 (3H, s), 4.29-4.38 (1H, m), 5.43 (2H, s), 6.22 (1H, d), 7.35-8.36 (8H, m) |
| | (CDCl₃, δ ppm): | |
| 15-5 | ¹H-NMR | 1.23 (3H, d), 1.95 (3H, s), 2.28 (3H, s), 2.50-2.66 (2H, m), 4.25-4.39 (1H, m), 4.40 (2H, s), 6.16 (1H, d), 7.24-8.27 (8H, m) |
| | (CDCl₃, δ ppm): | |
| 15-6 | ¹H-NMR | 1.23 (3H, d), 1.94 (3H, s), 2.33 (3H, s), 2.50-2.64 (2H, m), 4.28-4.37 (1H, m), 5.56 (1H, s), 6.20 (1H, d), 7.26-8.44 (8H, m) |
| | (CDCl₃, δ ppm): | |
| 15-7 | ¹H-NMR | 1.23 (3H, d), 1.95 (3H, s), 2.29 (3H, s), 2.51-2.66 (2H, m), 4.26-4.39 (1H, m), 4.40 (2H, s), 6.18 (1H, d), 7.25-8.28 (8H, m) |
| | (CDCl₃, δ ppm): | |
| 15-8 | ¹H-NMR | 1.30 and 1.41 (3H, d and d), 2.11-2.32 (6H, m), 2.67-2.88 (2H, m), 4.31-4.39 (1H, m), 4.40 (2H, s), 7.20-8.47 (8H, m) |
| | (CDCl₃, δ ppm): | |
| 15-9 | ¹H-NMR | 1.49 (3H, d), 2.28 (3H, s), 2.76 (3H, s), 4.40 (2H, s), 4.46-4.75 (1H, m), 6.61 (1H, d), 7.32-8.07 (8H, m) |
| | (CDCl₃, δ ppm): | |
| 15-13 | ¹H-NMR | 1.23 (3H, d), 1.95 (3H, s), 2.28 (3H, s), 2.42-2.71 (2H, br), 4.29-4.40 (1H, m), 4.40 (2H, s), 6.07-6.23 (1H, br), 7.24-8.27 (8H, m) |
| | (CDCl₃, δ ppm): | |
| 15-14 | ¹H-NMR | 1.23 (3H, d), 1.94 (3H, s), 2.32 (3H, s), 2.50-2.64 (2H, m), 4.28-4.34 (1H, m), 5.56 (1H, s), 6.13-6.36 (1H, br), 7.29-8.47 (8H, m) |
| | (CDCl₃, δ ppm): | |
| 15-16 | ¹H-NMR | 1.24 (3H, d), 2.28 (3H, s), 2.74 (3H, s), 3.10-3.35 (2H, m), 4.40 (2H, s), 4.56-4.62 (1H, m), 6.61 (1H, d), 7.28-7.93 (8H, m) |
| | (CDCl₃, δ ppm): | |
| 15-19 | ¹H-NMR | 1.48 (3H, d), 2.28 (3H, s), 2.78 (3H, s), 3.10-3.38 (2H, m), 4.41 (2H, s), 4.54-4.68 (1H, m), 6.76 (1H, d), 7.26-8.06 (8H, m) |
| | (CDCl₃, δ ppm): | |
| 15-20 | ¹H-NMR | 1.24 (3H, d), 1.96 (3H, s), 2.30 (3H, s), 2.49-2.66 (2H, m), 2.58 (3H, s), 4.25-4.40 (1H, m), 5.18 (2H, s), 6.34 (1H, d), 7.26-7.76 (5H, m), |
| | (CDCl₃, δ ppm): | 8.00 (1H, d), 8.40 (1H, bs) |
| 16-4 | ¹H-NMR | 1.26 (3H, d), 2.02 (3H, s), 2.31 (3H, s), 2.40-2.65 (2H, m), 3.96-4.07 (1H, m), 4.23-4.40 (1H, m), 4.58-4.70 (1H, m), 4.81-4.94 (1H, m), |
| | (CDCl₃, δ ppm): | 5.07-5.18 (1H, m), 6.18 (1H, bs), 7.06-7.17 (2H, m), 7.42-8.13 (4H, m), 8.34 (1H, bs) |
| 16-5 | ¹H-NMR (CDCl₃, δ ppm): | 1.24 (3H, d), 1.99 (3H, s), 2.31 (3H, s), 2.53-2.68 (2H, m), 3.78 (3H, s), 4.20 (1H, d), 4.29-4.42 (1H, m), 4.56 (1H, d), 5.52-5.59 (1H, m), 6.11 (1H, d), 7.03-7.12 |
| | | (2H, m), 7.45-7.79 (3H, m), 8.12 (1H, d), 8.35 (1H, bs) |
| 16-6 | ¹H-NMR | 1.24 (3H, d), 2.00 (3H, s), 2.13 (3H, s), 2.31 (3H, s), 2.52-2.68 (2H, m), 4.22 (1H, d), 4.29-4.41 (1H, m), 4.59 (1H, d), 5.64-5.71 (1H, m), |
| | (CDCl₃, δ ppm): | 6.12 (1H, d), 7.03-7.13 (2H, m), 7.45-7.79 (3H, m), 8.13 (1H, d), 8.36 (1H, bs) |
| 16-9 | ¹H-NMR | 1.27 (3H, d), 1.36 (6H, s), 1.99 (3H, s), 2.31 (3H, s), 2.51-2.68 (2H, m), 4.12 (2H, s), 4.28-4.41 (1H, m), 6.09-6.18 (1H, m), 6.18 (1H, bs), |
| | (CDCl₃, δ ppm): | 7.21-7.60 (4H, m), 7.75 (1H, d), 8.02 (1H, d), 8.27 (1H, bs) |
| 16-12 | ¹H-NMR | 1.22 (3H, d), 2.00 (3H, s), 2.12 (3H, s), 2.30 (3H, s), 2.53 (3H, s), 2.53-2.67 (2H, m), 4.22 (1H, d), 4.28-4.39 (1H, m), 4.59 (1H, d), 5.64-5.71 (1H, |
| | (CDCl₃, δ ppm): | m), 6.19 (1H, d), 7.04 (1H, s), 7.07 (1H, d), 7.40-7.62 (3H, m), 8.16 (1H, d), 8.29 (1H, bs) |
| 16-13 | ¹H-NMR | 1.23-1.27 (3H, m), 1.99-2.02 (3H, m), 2.28-2.32 (3H, m), 2.33-2.63 (2H, m), 2.53 (3H, s), 3.95-4.06 (1H, m), 4.23-4.37 (1H, m), 4.60-4.71 (1H, |
| | (CDCl₃, δ ppm): | m), 4.82-4.97 (1H, m), 5.06-5.18 (1H, m), 6.13 (1H, d), 7.05-7.14 (2H, m), 7.41-8.30 (5H, m) |
| 16-14 | ¹H-NMR | 1.18-1.25 (3H, m), 1.95-1.99 (3H, m), 2.27-2.34 (3H, m), 2.28-2.61 (2H, m), 3.29 (3H, s), 3.98-4.29 (2H, m), 4.57-4.68 (1H, m), 4.92-5.13 (2H, |
| | (CDCl₃, δ ppm): | m), 6.47 (1H, d), 7.09-7.18 (2H, m), 7.67-7.76 (1H, m), 7.90-8.09 (2H, m), 8.13-8.30 (2H, m) |

### BIOLOGICAL TEST EXAMPLE 1

### Test to Larva of Spodoptera litura

Solvent: dimethylformamide 3 parts by weight
Emulsifier: polyoxyethylene alkyl phenyl ether 1 part by weight

In order to prepare preparations of suitable active compounds, 1 part by weight of each of the active compounds was blended with the above amount of the solvent containing the above amount of the emulsifier, and the resulting mixture was diluted to a prescribed concentration with water.

Leaves of sweet potato were immersed in the tested liquid medication of a prescribed concentration diluted with water. After air drying of the liquid medication, the leaves were put into a petri dish of a diameter of 9 cm, and 10 Spodoptera litura third instar larvae were released in the petri dish, and the petri dish was placed in a constant temperature room of 25° C. Two days later and four days later, leaves of sweet potato were added. Seven days later, the number of dead insects was examined, and insect mortality was calculated.

In the present test, results of 2 petri dishes for each compound were averaged.

In the Biological Test Example 1, as the representative test examples, compounds of compound Nos. 1-5, 1-6, 1-7, 1-9, 1-10, 1-17, 1-20, 2-13, 2-14, 2-15, 4-4, 4-8, 4-9, 4-10, 4-11, 4-12, 4-13, 4-15, 5-3, 5-6, 5-10, 5-16, 5-17, 6-14, 6-15, 6-16, 6-20, 6-26, 6-34, 6-65, 7-7, 7-44, 8-2, 8-3, 8-4, 8-7, 8-8, 8-13, 8-14, 8-15, 13-3, 13-5, 13-7, 13-8, 13-10, 13-13, 13-17, 13-19, 13-23, 13-24, 13-26, 13-29, 13-30, 13-34, 13-35, 13-36, 13-37, 13-41, 13-45, 13-46, 13-48, 13-49, 13-51, 13-53, 13-54, 13-57, 13-59, 13-60, 13-61, 13-64, 13-65, 13-66, 13-67, 13-68, 13-73, 13-85, 13-86, 13-89, 13-90, 13-94, 13-108, 13-109, 13-114, 13-119, 13-121, 13-122, 13-123, 13-125, 13-127, 13-132, 13-137, 13-140, 13-147, 13-148, 13-149, 13-150, 13-158, 13-162, 13-166, 13-167, 13-172, 13-193, 13-195, 13-203, 13-204, 13-206, 13-212, 13-214, 13-215, 13-216, 13-218, 13-220, 13-223, 13-225, 13-233, 13-236, 13-240, 13-243, 13-244, 13-291, 13-292, 13-293, 13-294, 13-295, 13-296, 13-298, 13-300, 13-302, 13-307, 13-308, 13-310, 13-311, 13-317, 13-367, 14-4, 14-5, 14-6, 14-8, 14-9, 14-10, 14-11, 14-12, 14-13, 14-14, 14-17, 14-18, 14-19, 14-20, 14-21, 14-22, 14-23, 14-24, 14-25, 14-26, 14-29, 14-30, 14-32, 14-35, 14-36, 14-37, 14-41, 14-42, 14-44, 14-45, 14-46, 14-47, 14-48, 14-49, 14-50, 14-51, 14-52, 14-53, 14-54, 14-55, 14-57, 14-58, 14-59, 14-62, 14-63, 14-64, 14-66, 14-67, 14-68, 14-69, 14-70, 14-71, 14-72, 14-73, 14-74, 14-75, 14-76, 14-77, 14-78, 14-79, 14-80, 14-81, 14-112, 14-113, 14-114, 14-115, 14-116, 14-117, 14-118, 14-119, 14-120, 14-121, 14-122, 14-123, 14-124, 14-125, 14-126, 14-128, 14-130, 14-134, 14-136, 14-140, 14-141, 14-142, 14-143, 14-144, 14-148, 14-149, 14-152, 14-153, 14-155, 14-157, 14-160, 14-162, 14-163, 14-165, 14-166, 14-167, 14-168, 14-169, 14-170, 14-171, 14-172, 14-173, 14-174, 14-175, 14-176, 14-177, 14-178, 14-179, 14-180, 14-181, 14-182, 14-183, 14-185, 14-186, 14-187, 14-188, 14-189, 14-190, 14-191, 14-192, 14-193, 14-197, 14-198, 14-199, 14-200, 14-201, 14-202, 14-203, 14-204, 14-205, 14-206, 14-207, 14-208, 14-209, 14-210, 14-211, 14-212, 14-213, 14-214, 14-215, 14-216, 14-217, 14-218, 14-219, 14-220, 14-221, 14-222, 14-223, 14-224, 14-225, 14-227, 14-228, 14-229, 14-230, 14-231, 14-232, 14-233, 14-234, 14-235, 14-239, 14-241, 14-242, 14-244, 14-245, 14-246, 14-248, 14-249, 14-250, 14-251, 14-252, 14-253, 14-254, 14-255, 14-256, 14-257, 14-258, 14-259, 14-260, 14-261, 14-262, 14-265, 14-267, 14-292, 14-303, 14-312, 14-313, 14-314, 14-316, 14-338, 14-339, 14-340, 14-343, 14-344, 14-345, 14-346, 14-347, 14-351, 14-352, 14-354, 14-355, 14-356, 14-375, 14-378, 14-379, 14-380, 14-391, 14-426, 15-1, 15-2, 15-4, 15-5, 15-7, 15-8, 15-9, 15-13, 15-16, 15-18, 15-19, 15-20, 16-4, 16-5, 16-6, 16-9 and 16-14 exhibited 100% insect mortality at an active ingredient concentration of 20 ppm.

### BIOLOGICAL TEST EXAMPLE 2

### Test to Cnaphalocrocis medinalis Larva

### Preparation of Tested Liquid Medication

Solvent: dimethylformamide 3 parts by weight
Emulsifier: polyoxyethylene alkyl phenyl ether 1 part by weight

In order to prepare preparations of suitable active compounds, 1 part by weight of each of the active compounds was blended with the above amount of the solvent containing the above amount of the emulsifier, and the resulting mixture was diluted to a prescribed concentration with water.

As in the above biological test example 1, the solution diluted with water and having a prescribed concentration of the active compound was sprayed on potted paddy-rice (variety: Tamanishiki) such that the sprayed amount was 8 ml per a pot. After treated paddy-rice was air-dried, its stalk and phyllome part was cut so that the cut length was 4 to 5 cm. The stalk and phyllome part of the paddy-rice was put into a petri dish of 9 cm diameter in which a filter paper was placed and 2 mL of water was put. Five Cnaphalocrocis medinalis larvae of second instar were released in this petri dish, and the petri dish was placed in a constant temperature room of 25° C. Two days later and four days later, the rest (⅓ amount each) of the stalk and phyllome part of the paddy-rice was added. Seven days later, the number of dead insects was examined, and insect mortality was calculated. In the present test, results of 2 petri dishes for one compound were averaged.

In the above biological test example 2, as representative examples, compounds of compound Nos. 1-20, 2-7, 2-9, 2-10, 2-11, 2-12, 13-36, 13-53, 13-89, 13-90, 13-162, 14-2, 14-3, 14-4, 14-5, 14-6, 14-8, 14-9, 14-10, 14-11, 14-12, 14-22, 14-25, 14-26, 14-43, 14-46, 14-53, 14-62, 14-71, 14-76, 14-79, 14-163, 14-201, 14-205, 14-209, 14-212, 14-214, 14-228, 14-229, 14-230, 14-231, 14-232, 14-239, 14-240, 14-241, 14-242, 14-244, 14-245, 14-249, 14-250, 14-251, 14-252, 14-254, 14-255, 14-256, 14-257, 14-258, 14-259, 14-260, 14-261, 14-445 and 15-18, exhibited 100% insect mortality at an active ingredient concentration of 20 ppm.

### BIOLOGICAL TEST EXAMPLE 3

### Test to Myzus persicae

Solvent: acetone 78.0 parts by weight and dimethylformamide 1.5 parts by weight
Emulsifier: alkyl aryl polyglycol ether 0.5 parts by weight

In order to prepare preparations of suitable active compounds, 1 part by weight of each of the active compounds was blended with the above amount of the solvent containing the above amount of the emulsifier, and the resulting mixture was diluted to a prescribed concentration with water.

On a leaf disc of Brassica pekinensis, Myzus persicae including all growth stage was inoculated. The solution diluted with water and having a prescribed concentration of the active compound was sprayed by a spray gun. Insect mortality was calculated after prescribed days elapsed. 100% insect mortality means that all the individuals died, and 0% insect mortality means that all the individuals were alive.

As representative examples, compounds of compound Nos. 5-3, 5-4, 5-14, 5-15, 5-16, 5-18, 5-23, 7-7, 7-8, 7-13, 7-23, 7-150, 9-11, 9-12, 9-13, 9-21, 9-22, 11-1, 11-04, 11-27, 11-28, 11-29, 11-30, 13-7, 13-8, 13-58, 13-69, 13-70, 13-77, 13-80, 13-110, 13-128, 13-13, 13-34, 13-40, 13-43, 13-50, 13-51, 13-94, 13-134, 13-135, 13-136, 13-138, 13-139, 13-141, 13-149, 13-150, 13-156, 13-157, 13-159, 13-167, 13-172, 13-175, 13-178, 13-182, 13-186, 13-187, 13-188, 13-189, 13-232, 13-239, 13-247, 13-250, 13-254, 13-286, 13-287, 13-288, 13-290, 13-291, 13-292, 13-293, 13-294, 13-342, 13-343, 13-344, 13-345, 13-349, 13-363, 14-1, 14-5, 14-7, 14-13, 14-20, 14-23, 14-31, 14-35, 14-47, 14-48, 14-60, 14-61, 14-71, 14-76, 14-79, 14-163, 14-197, 14-262, 14-268, 14-271, 14-274, 14-286, 14-287, 14-288, 14-289, 14-290, 14-291, 14-299, 14-305, 14-306, 14-307, 14-308, 14-311, 14-328, 14-329, 14-330, 14-331, 14-334, 14-350, 14-364, 14-365, 14-366, 14-367, 14-368, 14-369, 14-370, 14-372, 14-373, 14-378, 14-379, 14-382, 14-384, 14-387, 14-389, 14-393, 14-394, 14-395, 14-397, 14-414, 15-2, 15-4, 15-5 and 15-15 exhibited a pesticide effect of 70% or more of insect mortality at a spraying amount of 100 g/ha.

### BIOLOGICAL TEST EXAMPLE 4

### Test to Myzus persicae resistant to organophosphorus pesticide and carbamate agent

Solvent: dimethylformamide 3 parts by weight
Emulsifier: polyoxyethylene alkyl phenyl ether 1 part by weight

In order to prepare preparations of suitable active compounds, 1 part by weight of each of the active compounds was blended with the above amount of the solvent containing the above amount of the emulsifier, and the resulting mixture was diluted to a prescribed concentration with water.

On egg plant seedlings planted in a plastic pot of a diameter of 6 cm, 30 bred Myzus persicae resistant to organophosphorus pesticide and carbamate agent per one seedling were inoculated. After 1 day elapsed since the inoculation, a sufficient amount of the solution diluted with water and having a prescribed concentration of the active compound was sprayed. After the spraying, the pot was allowed to stand in a greenhouse of 28° C. After seven days elapsed since the spraying, insect mortality was calculated. The test was carried out twice.

As representative test examples, compounds of compound Nos. 5-14, 5-15, 15-18, 5-22, 5-23, 6-14, 6-16, 6-20, 6-30, 6-34, 6-65, 8-14, 8-15, 13-13, 13-64, 13-119, 13-167, 13-172, 13-297, 13-298, 13-302, 13-303, 13-307, 13-310, 13-311, 13-344, 13-346, 13-367, 14-23, 14-47, 14-76, 14-79, 14-97, 14-148, 14-152, 14-165, 14-166, 14-167, 14-194, 14-198, 14-201, 14-220, 14-262, 14-337, 14-346, 14-370, 14-371, 14-372, 14-373, 14-374, 14-375, 14-376, 14-385, 14-386, 14-388, 14-392, 14-401, 14-407, 14-409, 14-411, 14-413, 14-416, 16-4, 16-12 and 16-14 exhibited a pesticide effect of 100% insect mortality at an effective ingredient concentration of 100 ppm.

### BIOLOGICAL TEST EXAMPLE 5

### Test to Phaedon cochleariae Preparation of Tested Liquid Medication

Solvent: acetone 78.0 parts by weight and dimethylformamide 1.5 parts by weight
Emulsifier: alkyl aryl polyglycol ether 0.5 parts by weight

In order to prepare preparations of suitable active compounds, 1 part by weight of each of the active compounds was blended with the above amount of the solvent containing the above amount of the emulsifier, and the resulting mixture was diluted to a prescribed concentration with water.

The solution diluted with water and having a prescribed concentration of the active compound was sprayed on a leaf disc of Brassica pekinensis by a spray gun. After air drying of the liquid medication, Phaedon cochleariae larvae were inoculated. After prescribed days elapsed, insect mortality was calculated. 100% Insect mortality means that all the individuals died, and 0% insect mortality means that all the individuals were alive.

As representative test examples, compounds of compound Nos. 11-5, 13-45, 13-178, 13-181, 13-252, 13-254, 13-259 and 15-15 showed a pesticide effect of 80% or more of insect mortality at a spraying amount of 500 g/ha.

### BIOLOGICAL TEST EXAMPLE 6

### Test to Spodoptera frugiperda

Solvent: acetone 78.0 parts by weight and dimethylformamide 1.5 parts by weight
Emulsifier: alkyl aryl polyglycol ether 0.5 parts by weight

In order to prepare preparations of suitable active compounds, 1 part by weight of each of the active compounds was blended with the above amount of the solvent containing the above amount of the emulsifier, and the resulting mixture was diluted to a prescribed concentration with water.

The solution diluted with water and having a prescribed concentration of the active compound was sprayed on leaves of Zea mays by a spray gun. After air drying of the liquid medication, Spodoptera frugiperda larvae were inoculated. After prescribed days elapsed, insect mortality was calculated. 100% Insect mortality means that all the individuals died, and 0% insect mortality means that all the individuals were alive.

As representative test examples, compounds of compound Nos. 11-5, 13-181, 13-191, 13-198, 13-251, 13-252, 13-253, 13-254, 13-255, 13-259, 13-261, 13-262, 13-263, 13-264, 13-265, 13-266, 13-269, 13-270, 13-271, 13-272, 13-275, 13-277, 14-300 and 15-15 showed a pesticide effect of 80% or more of insect morality at a spraying amount of 500 g/ha.

### BIOLOGICAL TEST EXAMPLE 7

### Test to Meloidogyne incognita

Solvent: acetone 80.0 parts by weight

In order to prepare preparation of suitable active compounds, 1 part by weight of each of the active compounds was blended with the above amount of the solvent containing the above amount of the emulsifier, and the resulting mixture was diluted to a prescribed concentration with water.

Sand, the solution diluted with water and having a prescribed concentration of the active compound, and a suspension containing larvae and eggs of Meloidogyne incognita, and seed of Lactuca sativa were put into a container. A pesticide effect was obtained by a node formation rate of root part after prescribed days elapsed. The pesticide effect 100% means that node was not found at all, and pesticide effect 0% means that the same number of nodes as that in the case where the above solution was not used was found.

As representative examples, compounds of compound Nos. 13-70, 13-110, 13-139, and 13-189 showed a pesticide effect of 80% or more at an active ingredient concentration of 20 ppm.

### BIOLOGICAL TEST EXAMPLE 8

### Test to Boophilus microplus

Solvent: dimethylsulfoxide

In order to prepare preparations of suitable active compounds, 10 mg of each of the active compounds was dissolved into 0.5 ml of the above solvent, and the resulting mixture was diluted to a prescribed concentration with the solvent.

To the abdomen of 5 Boophilus microplus blood-engorging female adults, the above-prepared compound solution was injected. Thereafter, they were transferred to a petri dish, bred in an incubator, and it was observed whether spawned eggs were dead or alive. After a prescribed time passed, mortality was obtained. 100% mortality means that all the eggs did not hatch, and 0% mortality means that all the eggs hatched.

In the above biological tests, the compounds of 13-50, 13-64, 13-70, 13-128, 13-132, 13-135, 13-137, 13-157, 13-190, 13-247, 13-255, 13-263, 13-265, 13-271, 13-292, 13-295, 13-308, 14-22, 14-35, 14-43, 14-53, 14-62, 14-118, 14-120, 14-124, 14-128, 14-165, 14-166, 14-170, 14-174, 14-185, 14-189, 14-193, 14-199, 14-202, 14-204, 14-223, 14-251, 14-253, 14-256, 14-286, 14-287, 14-288, 14-290, 14-343, 14-344, 14-352, 14-374, 14-379, 14-401 and 15-13 showed insecticide activity of 80% or more at a treated amount of 20 μg/insect.

### BIOLOGICAL TEST EXAMPLE 9

### Test to Lucilia cuprina

Solvent: dimethylsulfoxide

In order to prepare preparations of suitable active compounds, 10 mg of each of the active compounds was dissolved into 0.5 ml of the above solvent, and the resulting mixture was diluted to a prescribed concentration with water.

About 20 to 30 larvae of Lucilia cuprina were put into the test tube in which 1 cm³ of horse minced meat and 0.5 ml of the aqueous solution of the compound prepared in the above were put. After a fixed time passed, the mortality of the Lucilia cuprina was obtained. 100% mortality means that all the individuals were dead, and 0% mortality means that all the individuals were alive.

In the above biological test, compounds of 13-50, 13-64, 13-70, 13-128, 13-132, 13-135, 13-137, 13-157, 13-190, 13-247, 13-292, 13-295, 13-308, 14-22, 14-35, 14-43, 14-53, 14-62, 14-120, 14-124, 14-128, 14-165, 14-166, 14-170, 14-174, 14-185, 14-189, 14-193, 14-199, 14-202, 14-204, 14-223, 14-251, 14-253, 14-256, 14-286, 14-287, 14-288, 14-290, 14-343, 14-344, 14-352, 14-374, 14-379, 14-401 and 15-13 showed insecticide activity of 80% or more at an effective ingredient concentration 100 ppm.

### BIOLOGICAL TEST EXAMPLE 10

### Test to Musca domestica

### Preparation of Tested Liquid Medication

Solvent: dimethylsulfoxide

In order to prepare preparations of suitable active compounds, 10 mg of each of the active compounds was dissolved into 0.5 ml of the above solvent, and the resulting mixture was diluted to a prescribed concentration with water.

As a preparative stage of the test, a fixed size of sponge was soaked in a mixture of sugar and the compound aqueous solution prepared in the above, and was placed in a test container. Ten adults of Musca domestica were put into the container, and the container was closed with a cap having ventilation holes. After a fixed time passed, a mortality rate of Musca domestica was acquired. 100% mortality means that all the individuals were dead, and 0% mortality means that all the individuals were alive.

In the above biological tests, the compounds of 13-50, 13-70, 13-128, 13-132, 13-135, 13-137, 13-157, 13-190, 13-247, 13-292, 13-295, 13-308, 14-43, 14-53, 14-118, 14-124, 14-165, 14-166, 14-170, 14-174, 14-189, 14-193, 14-199, 14-202, 14-223, 14-253, 14-286, 14-287, 14-288, 14-290, 14-343, 14-344, 14-352, 14-374, 14-379 and 15-13 showed insecticide activity of 70% or more at an effective ingredient concentration of 100 ppm.

### FORMULATION EXAMPLE 1 (GRANULE)

Twenty five parts of water was added to a mixture including 10 parts of the inventive compound (No. 8), 30 parts of bentonite (montmorillonite), 58 parts of talc, and 2 parts of lignin sulfonic acid salt, and the resulting mixture was kneaded well to form granules of 10 to 40 mesh by an extruding granulator, and dried at 40 to 50° C.

### FORMULATION EXAMPLE 2 (GRANULE)

Ninety-five parts of clay mineral particles having particle size distribution in the range of 0.2 to 2 mm were put into a rotary mixer. After 5 parts of the inventive compound (No. 11) were sprayed together with a liquid diluting agent under rotation to uniformly wet the particles, the particles were dried at 40 to 50° C. to form granules.

### FORMULATION EXAMPLE 3 (EMULSION)

Thirty parts of the inventive compound (No. 12), 55 parts of xylene, 8 parts of polyoxyethylene alkyl phenyl ether, and 7 parts of calcium alkylbenzene sulfonate were stirred and blended to form an emulsion.

### FORMULATION EXAMPLE 4 (WETTABLE POWDER)

Fifteen parts of the inventive compound (No. 15), 80 parts of a mixture of white carbon (hydrous amorphous silica fine powder) and powder clay at a ratio of 1:5, 2 parts of sodium alkylbenzene sulfonate, and 3 parts of a condensed product of formalin and sodium alkylnaphthalene sulfonate were crushed and blended to form wettable powder.

### FORMULATION EXAMPLE 5 (WATER-DISPERSIBLE GRANULE)

Twenty parts of the inventive compound (No. 16), 30 parts of sodium lignin sulfonate, 15 parts of bentonite, and 35 parts of calcined diatomaceous earth powder were well mixed, and water was added thereto, and the resulting mixture was extruded through a screen of 0.3 mm, and dried to form water-dispersible granules.
Novel benzenedicarboxamides of the present invention have superior insecticide action as insecticides as shown in the above examples.

1. A benzenedicarboxamide derivative represented by the following formula: 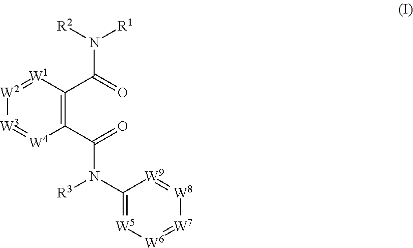
wherein R¹ represents a hydrogen atom, or alkyl, alkenyl, alkynyl, cycloalkyl, alkoxy, dialkylamino, carboxyalkyl, formylalkyl, hydroxyiminoalkyl, hydroxyalkyl, alkoxyalkyl, aminosulfonylalkyl, alkylaminosulfonylalkyl, alkylthioalkyl, alkylsulfinylalkyl, alkylsulfonylalkyl, cycloalkylthioalkyl, cycloalkylsulfinylalkyl, cycloalkylsulfonylalkyl, trialkylsilylalkyl, alkoxycarbonylmethylaminocarbonylalkyl, alkylcarbamoyloxyalkyl, monoalkylcarbamoylalkyl, monoalkenylcarbamoylalkyl, monoalkynylcarbamoylalkyl, cycloalkylcarbamoylalkyl, alkoxycarbonylaminoalkyl, dialkylaminosulfonylalkyl, benzyloxycarbonylalkyl, benzyloxyalkyl, alkylthioaryl, alkylsulfinylaryl, alkylsulfonylaryl, alkylthioheteroaryl, alkylsulfinylheteroaryl, alkylsulfonylheteroaryl, cycloalkylalkyl, heterocyclyl or heterocyclylalkyl which may be substituted,
R² and R³ each independently represent a hydrogen atom, or alkyl, alkenyl, alkynyl, alkoxyalkyl or alkylthioalkyl which may be substituted,
R¹ and R² together with a nitrogen atom to which they are attached may form a 5- or 6-membered heterocyclic group,
W¹ represents a nitrogen atom or C—X¹,
W² represents a nitrogen atom or C—X²,
W³ represents a nitrogen atom or C—X³,
W⁴ represents a nitrogen atom or C—X⁴,
X¹, X², X³ and X⁴, which may be identical or different, represent a hydrogen atom, nitro, formyl, amino, cyano, halogen, carbamoyl, or alkyl, haloalkyl, alkylcarbonyl, alkoxycarbonyl, acylamino, alkoxy, haloalkoxy, alkylthio, alkylsulfinyl, alkylsulfonyl, haloalkylthio, haloalkylsulfinyl, haloalkylsulfonyl, cycloalkylthio, cycloalkylsulfinyl, cycloalkylsulfonyl, cycloalkylalkylthio, cycloalkylalkylsulfinyl, cycloalkylalkylsulfonyl, alkylsulfonyloxy, haloalkylsulfonyloxy, monoalkylaminosulfonyl, dialkylaminosulfonyl, monoalkylamino, dialkylamino, monoalkylcarbamoyl, dialkylcarbamoyl, alkenyl, alkenyloxy, alkynyl, alkynyloxy, cycloalkyl, cycloalkyloxy, aryl, aryloxy, arylthio, arylalkyl, arylalkoxy, arylalkylthio, heteroaryl, heterocyclyl, heterocyclyloxy, heterocyclylthio or heterocyclylalkoxy which may be substituted, adjacent W¹ and W², W² and W³ or W³ and W⁴ may together form a 5- or 6-membered carbon ring or a heterocyclic group,
W⁵ represents a nitrogen atom, C—Y¹ or C-(A)r-Q,
W⁶ represents a nitrogen atom, C—Y² or C-(A)r-Q,
W⁷ represents a nitrogen atom, C—Y³ or C-(A)r-Q,
W⁸ represents a nitrogen atom, C—Y⁴ or C-(A)r-Q,
W⁹ represents a nitrogen atom, C—Y⁵ or C-(A)r-Q,
at least one of W⁵ to W⁹ necessarily represents C-(A)r-Q, Y¹, Y², Y³, Y⁴ and Y⁵, which may be identical or different, represent a hydrogen atom, nitro, formyl, carbamoyl, amino, cyano, halogen, or alkyl, haloalkyl, alkylcarbonyl, alkoxycarbonyl, acylamino, alkoxy, haloalkoxy, alkoxycarbonylalkyl, alkylthio, alkylsulfinyl, alkylsulfonyl, haloalkylthio, alkylthioalkyl, monoalkylamino, dialkylamino, monoalkylcarbamoyl, dialkylcarbamoyl, alkenyl, alkenyloxy, alkynyl, alkynyloxy, cycloalkyl, cycloalkyloxy, cycloalkylalkyl, cycloalkylalkoxy, aryl, aryloxy, arylthio, arylalkyl, arylalkoxy, arylalkylthio, heteroaryl, heterocyclyl, heterocyclyloxy, heterocyclylthio or heterocyclylalkoxy which may be substituted,
A represents O, S, SO, SO₂, NH, N(CH₃), CH(CN), C(═N—OCH), C(═O), CH(OH), CH(CH₃), CH(CF₃), C(CF₃)₂, CH(CO₂CH₃) or CH(CO₂C₂H₅), or alkylene which may be interrupted by O, S, SO, SO₂, NH, N(CH₃), CH(CN), C(═N—OCH₃), C(═O), CH(OH), CH(CH₃), CH(CF₃), C(CF₃)₂, CH(CO₂CH₃) or CH(CO₂C₂H₅),
r represents 0, 1, 2, 3, 4 or 5, and
Q represents a 5- or 6-membered heterocyclic group which may be substituted.

2. The compound of claim 1, wherein R¹ represents a hydrogen atom, or C₁₋₈ alkyl, C₂₋₈ alkenyl, C₂₋₈ alkynyl, C₃₋₈ cycloalkyl, C₁₋₈ alkoxy, C₂₋₈ (total number of carbon atoms) dialkylamino, carboxy-C₁₋₆ alkyl, formyl-C₁₋₆ alkyl, hydroxyimino-C₁₋₆ alkyl, hydroxy-C₁₋₆ alkyl, C₂₋₈ (total number of carbon atoms) alkoxyalkyl, aminosulfonyl-C₁₋₆ alkyl, C₂₋₁₀ (total number of carbon atoms) alkylaminosulfonylalkyl, C₂₋₁₀ (total number of carbon atoms) alkylthioalkyl, C₂₋₁₀ (total number of carbon atoms) alkylsulfinylalkyl, C₂₋₁₀ (total number of carbon atoms) alkylsulfonylalkyl, C₄₋₁₂ (total number of carbon atoms) cycloalkylthioalkyl, C₄₋₁₂ (total number of carbon atoms) cycloalkylsulfinylalkyl, C₄₋₁₂ (total number of carbon atoms) cycloalkylsulfonylalkyl, C₄₋₁₀ (total number of carbon atoms) trialkylsilylalkyl, C₂₋₁₀ (total number of carbon atoms) alkoxycarbonylmethylaminocarbonylalkyl, C₂₋₁₀ (total number of carbon atoms) alkylcarbamoyloxyalkyl, C₂₋₁₀ (total number of carbon atoms) monoalkylcarbamoylalkyl, C₃₋₁₀ (total number of carbon atoms) monoalkenylcarbamoylalkyl, C₄₋₁₀ (total number of carbon atoms) cycloalkylcarbamoylalkyl, C₂₋₁₀ (total number of carbon atoms) alkoxycarbonylaminoalkyl, C₂₋₁₀ (total number of carbon atoms) dialkylaminosulfonylalkyl, benzyloxycarbonyl-C₁₋₆ alkyl, benzyloxy-C₁₋₆ alkyl, C₁₋₄ alkylthioaryl, C₁₋₄ alkylsulfinylaryl, C₁₋₄ alkylsulfonylaryl, C₁₋₄ alkylthioheteroaryl, C₁₋₄ alkylsulfinylheteroaryl, C₁₋₄ alkylsulfonylheteroaryl, C₃₋₁₀ (total number of carbon atoms) cycloalkylalkyl which may be substituted, or 5- or 6-membered heterocyclyl or 5- or 6-membered heterocyclyl-C₁₋₆ alkyl which may be substituted,
R¹ and R² together with a nitrogen atom to which they are attached may form a saturated 5- or 6-membered heterocyclic group constituted by a combination of a carbon atom, an oxygen atom and a sulfur atom,
R² and R³ represent a hydrogen atom, or C₁₋₆ alkyl, C₂₋₆ alkenyl, C₂₋₆ alkynyl, C₂₋₈ (total number of carbon atoms) alkoxyalkyl or C₂₋₈ (total number of carbon atoms) alkylthioalkyl which may be substituted,
W¹ represents a nitrogen atom or C—X¹,
W² represents a nitrogen atom or C—X²,
W³ represents a nitrogen atom or C—X³,
W⁴ represents a nitrogen atom or C—X⁴, X¹, X², X³ and X⁴, which may be identical or different, represent a hydrogen atom, nitro, formyl, amino, cyano, halogen, carbamoyl, or C₁₋₆ alkyl, C₁₋₆ haloalkyl, C₁₋₆ alkylcarbonyl, C₁₋₆ alkoxycarbonyl, C₁₋₆ acylamino, C₁₋₆ alkoxy, C₁₋₆ haloalkoxy, C₁₋₆ alkylthio, C₁₋₆ alkylsulfinyl, C₁₋₆ alkylsulfonyl, C₁₋₆ haloalkylthio, C₁₋₆ haloalkylsulfinyl, C₁₋₆ haloalkylsulfonyl, C₃₋₈ cycloalkylthio, C₃₋₈ cycloalkylsulfinyl, C₃₋₈ cycloalkylsulfonyl, C₄₋₁₀ (total number of carbon atoms) cycloalkylalkylthio, C₄₋₁₀ (total number of carbon atoms) cycloalkylalkylsulfinyl, C₄₋₁₀ (total number of carbon atoms) cycloalkylalkylalkylsulfonyl, C₁₋₆ alkylsulfonyloxy, C₁₋₆ haloalkylsulfonyloxy, C₁₋₆ monoalkylaminosulfonyl, C₂₋₈ (total number of carbon atoms) dialkylaminosulfonyl, C₁₋₆ monoalkylamino, C₂₋₈ (total number of carbon atoms) dialkylamino, C₁₋₆ monoalkylcarbamoyl, C₂₋₈ (total number of carbon atoms) dialkylcarbamoyl, C₂₋₆ alkenyl, C₂₋₆ alkenyloxy, C₂₋₆ alkynyl, C₂₋₆ alkynyloxy, C₃₋₈ cycloalkyl, C₃₋₈ cycloalkyloxy which may be substituted, or aryl, aryloxy, arylthio, aryl C₁₋₄ alkyl, aryl C₁₋₄ alkoxy which may be substituted, aryl C₁₋₄ alkylthio, heteroaryl, heterocyclyl, heterocyclyloxy, heterocyclylthio or heterocyclyl C₁₋₄ alkoxy which may be substituted by nitro, formyl, cyano, halogen, and C₁₋₄ alkyl, C₁₋₄ haloalkyl, C₁₋₄ alkylcarbonyl, C₁₋₄ alkoxycarbonyl, C₁₋₄ alkoxy, C₁₋₄ haloalkoxy, C₁₋₄ alkylthio or C₁₋₄ haloalkylthio which may be substituted, adjacent W¹ and W², W² and W³ or W³ and W⁴ may form a 5- or 6-membered carbon ring or a heterocyclic group constituted by a combination of oxygen, sulfur, nitrogen which may be substituted by C₁₋₂ alkyl, and carbon which may be substituted by halogen,
W⁵ represents a nitrogen atom, C—Y¹ or C-(A)r-Q,
W⁶ represents a nitrogen atom, C—Y² or C-(A)r-Q,
W⁷ represents a nitrogen atom, C—Y³ or C-(A)r-Q,
W⁸ represents a nitrogen atom, C—Y⁴ or C-(A)r-Q,
W⁹ represents a nitrogen atom, C—Y⁵ or C-(A)r-Q,
among these, at least one of W⁵ to W⁹ necessarily represents C-(A)r-Q,
Y¹, Y³, Y⁴ and Y⁵, which may be identical or different, represent a hydrogen atom, nitro, formyl, carbamoyl, amino, cyano, halogen, or C₁₋₆ alkyl, C₁₋₆ haloalkyl, C₁₋₆ alkylcarbonyl, C₁₋₆ alkoxycarbonyl, C₁₋₆ acylamino, C₁₋₆ alkoxy, C₁₋₆ haloalkoxy, C₂₋₈ (total number of carbon atoms) alkoxycarbonylalkyl, C₁₋₆ alkylthio, C₁₋₆ alkylsulfinyl, C₁₋₆ alkylsulfonyl, C₁₋₆ haloalkylthio, C₂₋₈ (total number of carbon atoms) alkylthioalkyl, C₁₋₆ monoalkylamino, C₂₋₈ (total number of carbon atoms) dialkylamino, C₁₋₆ monoalkylcarbamoyl, C₂₋₈ (total number of carbon atoms) dialkylcarbamoyl, C₂₋₆ alkenyl, C₂₋₆ alkenyloxy, C₂₋₆ alkynyl, C₂₋₆ alkynyloxy, C₃₋₈ cycloalkyl, C₃₋₈ cycloalkyloxy, C₄₋₉ (total number of carbon atoms) cycloalkylalkyl, C₄₋₉ (total number of carbon atoms) cycloalkylalkoxy which may be substituted, or aryl, aryloxy, arylthio, aryl C₁₋₄ alkyl, aryl C₁₋₄ alkoxy, aryl C₁₋₄ alkylthio, heteroaryl, heterocyclyl, heterocyclyloxy, heterocyclylthio or heterocyclyl C₁₋₄ alkoxy which may be substituted by at least one selected from the group consisting of formyl, cyano, halogen, C₁₋₄ alkyl, C₁₋₄ haloalkyl, C₁₋₄ alkylcarbonyl, C₁₋₄ alkoxycarbonyl, C₁₋₄ alkoxy, C₁₋₄ haloalkoxy, C₁₋₄ alkylthio and C₁₋₄ haloalkylthio,
A represents O, S, SO, SO₂, NH, N(CH₃), CH(CH₃), CH(C₂H₅), C(═O), C(═N—OCH₃), CH(OH), CH(CN), CH(CF₃), C(CF₃)₂, CH(CO₂CH₃) or CH(CO₂C₂H₅), or C₁₋₄ alkylene which may be interrupted by O, S, SO, SO₂, NH, N(CH₃), CH(CH₃), CH(C₂H₅), C(═O), C(═N—OCH₃), CH(OH), CH(CN), CH(CF₃), C(CF₃)₂, CH(CO₂CH₃) or CH(CO₂C₂H₅),
r represents 0, 1, 2, 3 or 4, and
Q represents a 5- or 6-membered heterocyclic group which has at least one heteroatom selected from a nitrogen atom, an oxygen atom and a sulfur atom and is selected from the following Q1 to Q67: 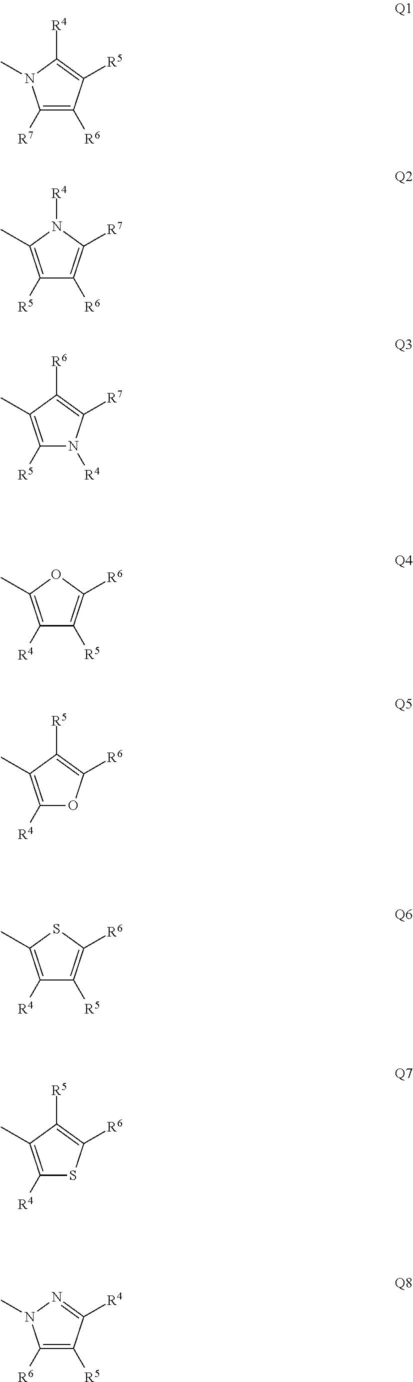 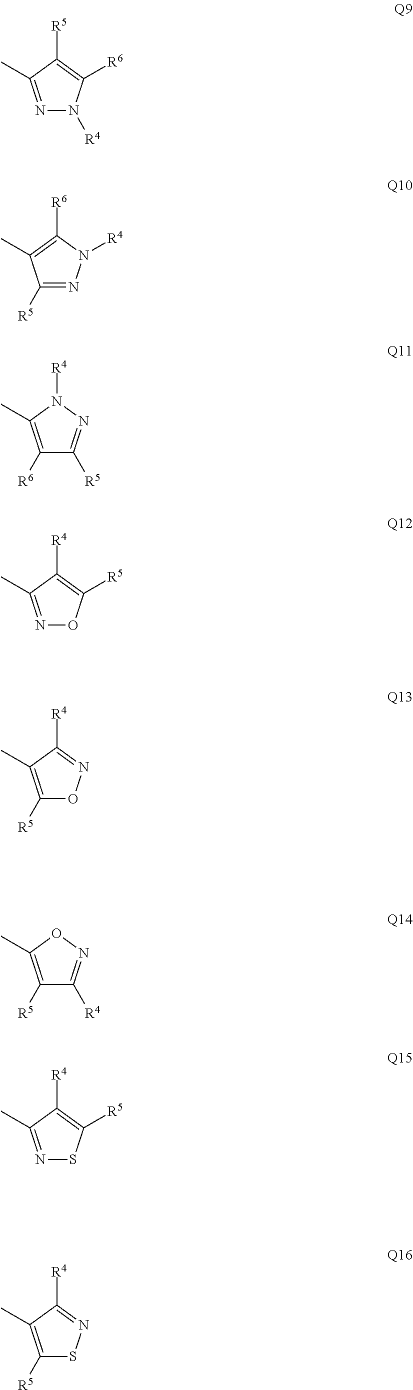 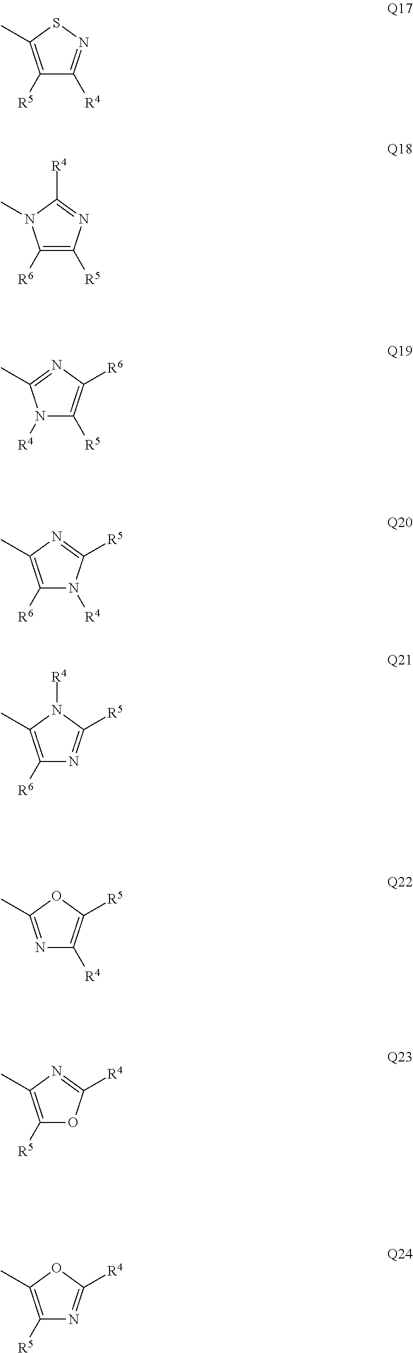 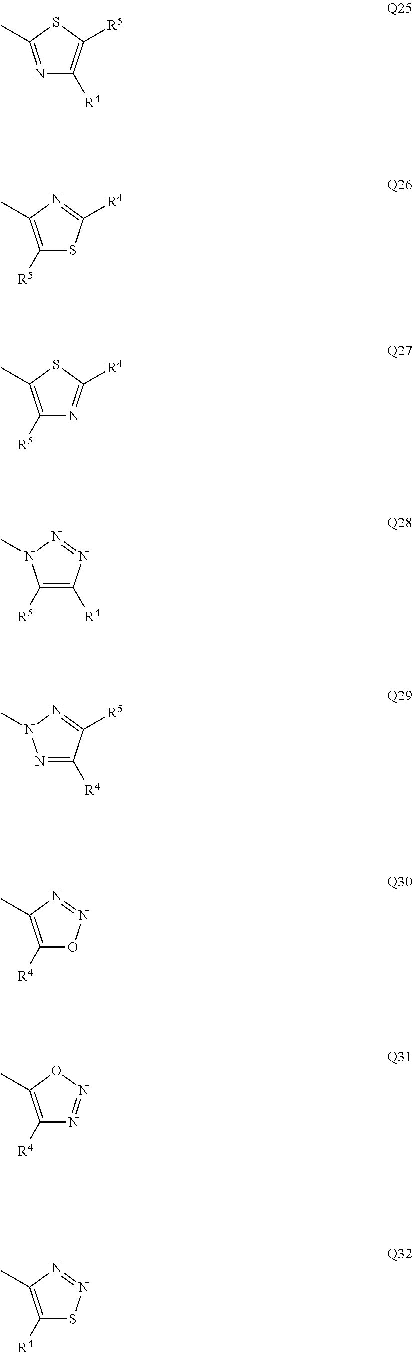 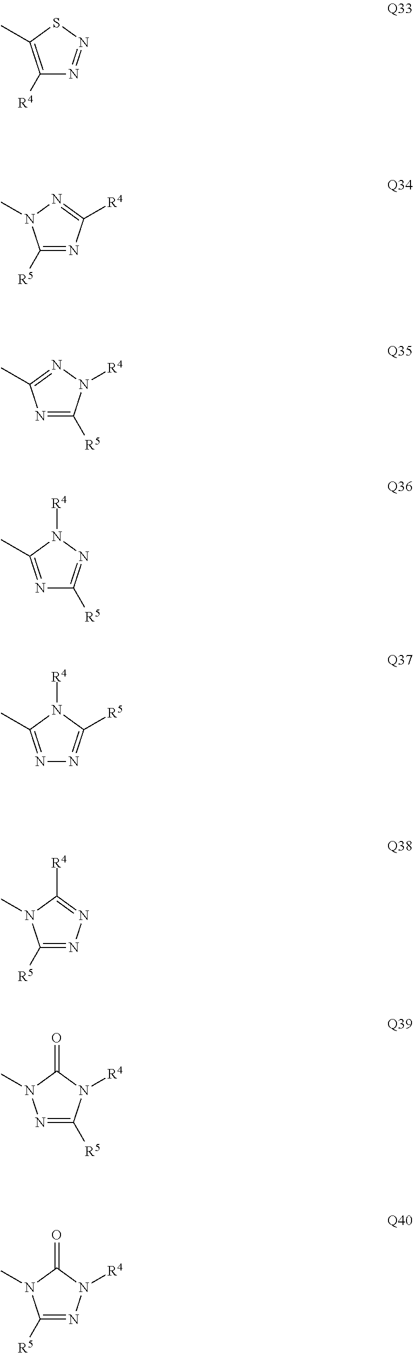 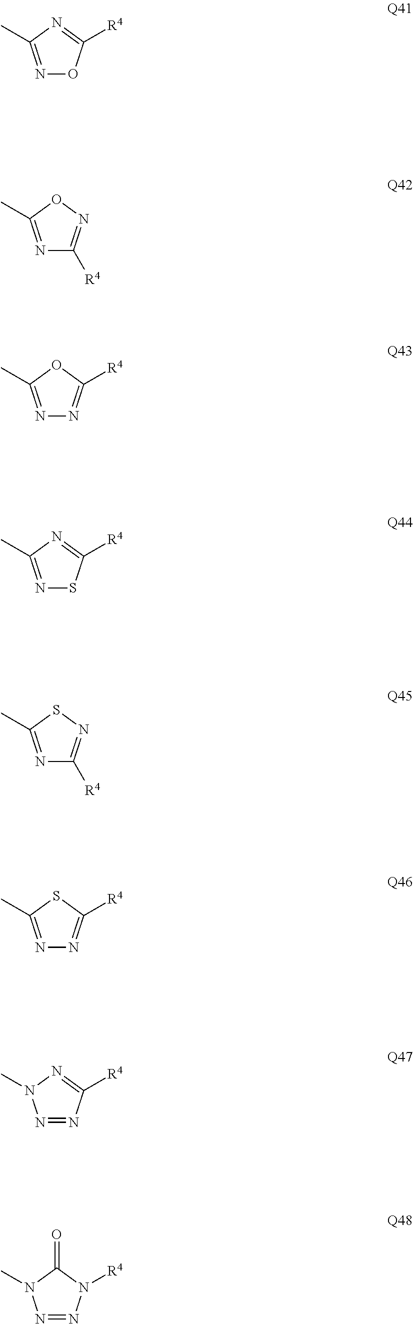 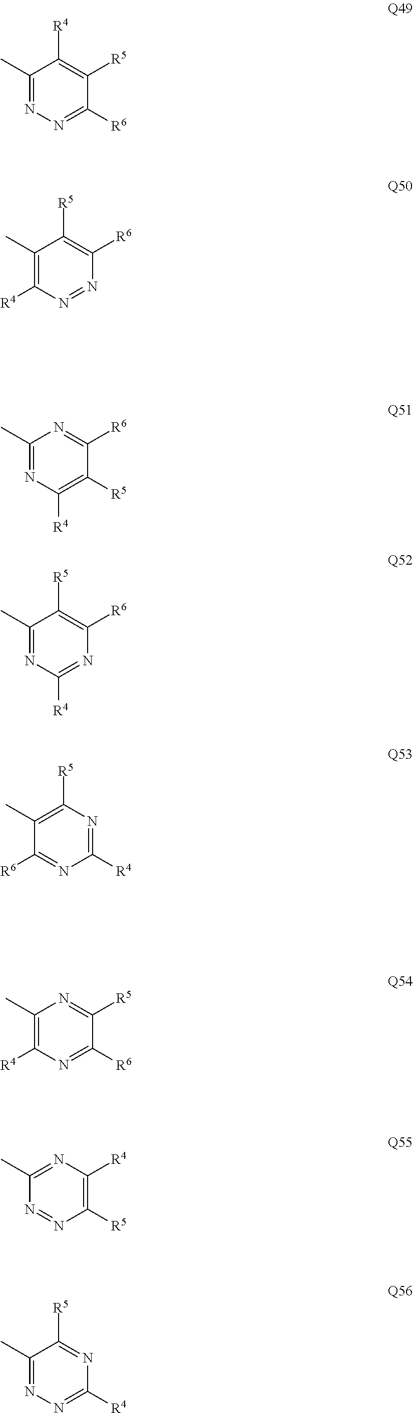 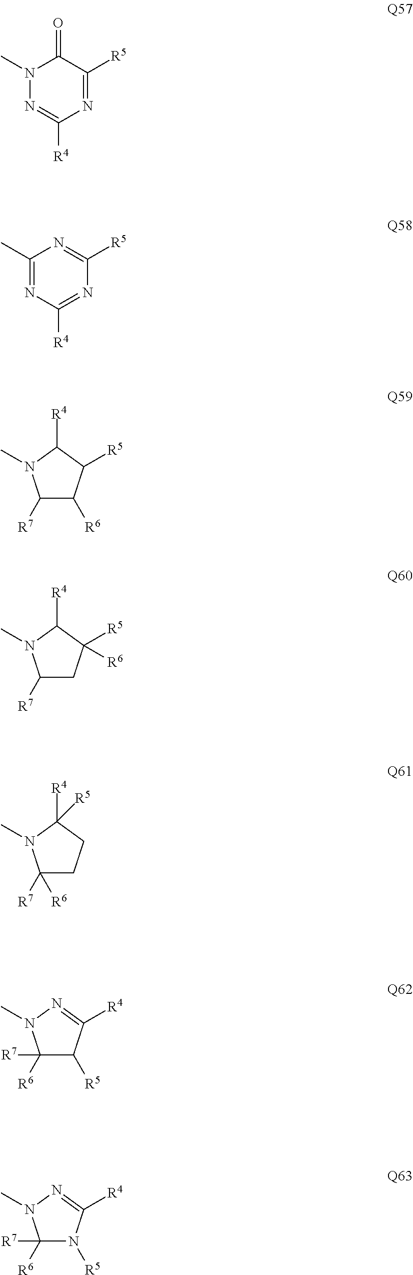 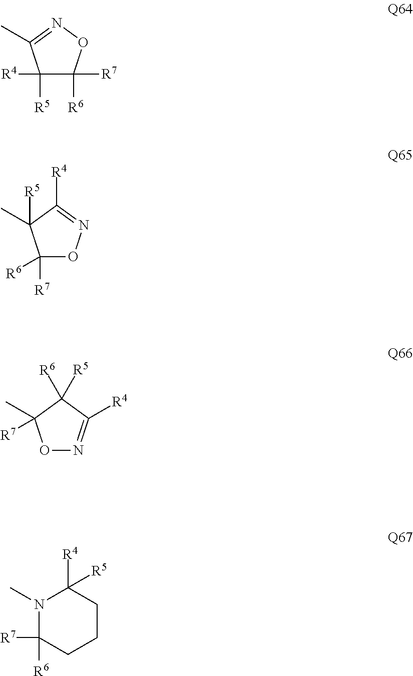
wherein R⁴, R⁵, R⁶ and R⁷, which may be identical or different, represent a hydrogen atom, halogen, amino, cyano, nitro, or C₁₋₆ alkyl, C₁₋₁₀ haloalkyl, C₂₋₆ alkynyl, C₃₋₈ cyaloalkyl, C₁₋₆ alkoxy, C₁₋₆ haloalkoxy, C₁₋₆ alkylthio, C₁₋₆ haloalkylthio, C₁₋₆ alkylsulfinyl, C₁₋₆ haloalkylsulfinyl, C₁₋₆ alkylsulfonyl, C₁₋₆ haloalkylsulfonyl, C₁₋₆ alkoxycarbonyl, C₁₋₆ alkylcarbonyl, C₁₋₆ haloalkylcarbonyl, C₁₋₆ acylamino, C₁₋₆ haloacylamino, monoalkylcarbamoyl, C₂₋₈ (total number of carbon atoms) dialkylcarbamoyl, hydroxyimino-C₁₋₆ alkyl, C₂₋₈ (total number of carbon atoms) alkoxyiminoalkyl, hydroxyimino-C₁₋₆ haloalkyl, C₂₋₈ (total number of carbon atoms) alkoxyiminohaloalkyl which may be substituted, or phenylcarbamoyl which may be substituted by at least one selected from the group consisting of halogen, cyano, C₁₋₄ alkyl, C₁₋₆ haloalkyl, C₁₋₆ alkoxy, C₁₋₄ haloalkoxy, C₁₋₄ alkylthio and C₁₋₄ haloalkylthio, or a phenyl or heterocyclic group which may be substituted by at least one selected from the group consisting of halogen, cyano, and C₁₋₆ alkyl, C₂₋₆ alkenyl, C₁₋₆ haloalkyl, C₁₋₆ alkoxy, C₁₋₆ haloalkoxy, C₁₋₆ alkylthio, C₁₋₆ haloalkylthio, C₂₋₅ (total number of carbon atoms) alkylthioalkyl, C₁₋₆ alkylcarbonyl and C₁₋₆ alkoxycarbonyl which may be substituted.

3. The compound of claim 1, wherein R¹ represents a hydrogen atom, or C₁₋₆ alkyl, C₂₋₆ alkenyl, C₂₋₆ alkynyl, C₃₋₆ cycloalkyl, C₁₋₆ alkoxy, C₂₋₆ (total number of carbon atoms) dialkylamino, carboxy-C₁₋₄ alkyl, formyl-C₁₋₄ alkyl, hydroxyimino-C₁₋₄ alkyl, hydroxy-C₁₋₄ alkyl, C₂₋₆ (total number of carbon atoms) alkoxyalkyl, aminosulfonyl-C₁₋₄ alkyl, C₂₋₈ (total number of carbon atoms) alkylaminosulfonylalkyl, C₂₋₈ (total number of carbon atoms) alkylthioalkyl, C₂₋₈ (total number of carbon atoms) alkylsulfinylalkyl, C₂₋₈ (total number of carbon atoms) alkylsulfonylalkyl, C₄₋₁₀ (total number of carbon atoms) cycloalkylthioalkyl, C₄₋₁₀ (total number of carbon atoms) cycloalkylsulfinylalkyl, C₄₋₁₀ (total number of carbon atoms) cycloalkylsulfonylalkyl, C₄₋₈ (total number of carbon atoms) trialkylsilylalkyl, C₂₋₈ (total number of carbon atoms) alkoxycarbonylmethylaminocarbonylalkyl, C₂₋₈ (total number of carbon atoms) alkylcarbamoyloxyalkyl, C₂₋₈ (total number of carbon atoms) monoalkylcarbamoylalkyl, C₃₋₈ (total number of carbon atoms) monoalkenylcarbamoylalkyl, C₄₋₈ (total number of carbon atoms) cycloalkylcarbamoylalkyl, C₂₋₈ (total number of carbon atoms) alkoxycarbonylaminoalkyl, C₂₋₈ (total number of carbon atoms) dialkylaminosulfonylalkyl, benzyloxycarbonyl-C₁₋₄ alkyl, benzyloxy-C₁₋₄ alkyl, C₁₋₂ alkylthioaryl, C₁₋₂ alkylsulfinylaryl, C₁₋₂ alkylsulfonylaryl, C₁₋₂ alkylthioheteroaryl, C₁₋₂ alkylsulfinylheteroaryl, C₁₋₂ alkylsulfonylheteroaryl, C₃₋₈ (total number of carbon atoms) cycloalkylalkyl, 5- or 6-membered heterocyclyl or 5- or 6-membered heterocyclyl-C₁₋₄ alkyl which may be substituted, R¹ and R² together with a nitrogen atom to which they are attached may form a 5- or 6-membered heterocyclic group: 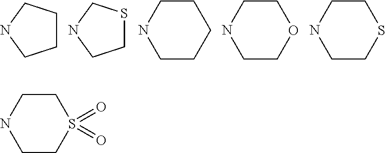
R² and R³ represent a hydrogen atom, or C₁₋₄ alkyl, C₂₋₄ alkenyl, C₂₋₄ alkynyl, C₂₋₆ (total number of carbon atoms) alkoxyalkyl or C₂₋₆ (total number of carbon atoms) alkylthioalkyl which may be substituted,
W¹ represents a nitrogen atom or C—X¹,
W² represents a nitrogen atom or C—X²,
W³ represents a nitrogen atom or C—X³,
W⁴ represents a nitrogen atom or C—X⁴, X¹, X², X³ and X⁴, which may be identical or different, represent a hydrogen atom, nitro, formyl, amino, cyano, halogen, carbamoyl, or C₁₋₄ alkyl, C₁₋₄ haloalkyl, C₁₋₄ alkylcarbonyl, C₁₋₄ alkoxycarbonyl, C₁₋₄ acylamino, C₁₋₄ alkoxy, C₁₋₄ haloalkoxy, C₁₋₄ alkylthio, C₁₋₄ alkylsulfinyl, C₁₋₄ alkylsulfonyl, C₁₋₄ haloalkylthio, C₁₋₄ haloalkylsulfinyl, C₁₋₄ haloalkylsulfonyl, C₃₋₆ cycloalkylthio, C₃₋₆ cycloalkylsulfinyl, C₃₋₆ cycloalkylsulfonyl, C₄₋₈ (total number of carbon atoms) cycloalkylalkylthio, C₄₋₈ (total number of carbon atoms) cycloalkylalkylsulfinyl, C₄₋₈ (total number of carbon atoms) cycloalkylalkylsulfonyl, C₁₋₄ alkylsulfonyloxy, C₁₋₄ haloalkylsulfonyloxy, C₁₋₄ monoalkylaminosulfonyl, C₂₋₆ (total number of carbon atoms) dialkylaminosulfonyl, C₁₋₄ monoalkylamino, C₂₋₆ (total number of carbon atoms) dialkylamino, C₁₋₄ monoalkylcarbamoyl, C₂₋₆ (total number of carbon atoms) dialkylcarbamoyl, C₂₋₄ alkenyl, C₂₋₄ alkenyloxy, C₂₋₄ alkynyl, C₂₋₄ alkynyloxy, C₃₋₆ cycloalkyl, C₃₋₆ cycloalkyloxy which may be substituted, or aryl, aryloxy, arylthio, aryl C₁₋₂ alkyl, aryl C₁₋₂ alkoxy, aryl C₁₋₂ alkylthio, heteroaryl, heterocyclyl, heterocyclyloxy, heterocyclylthio or heterocyclyl C₁₋₂ alkoxy which may be substituted by nitro, formyl, cyano, halogen, C₁₋₂, alkyl, C₁₋₂ haloalkyl, C₁₋₂ alkylcarbonyl, C₁₋₂ alkoxycarbonyl, C₁₋₂ alkoxy, C₁₋₂ haloalkoxy, C₁₋₂ alkylthio or C₁₋₂ haloalkylthio, adjacent W¹ and W², W² and W³ or W³ and W⁴ may together form a carbon ring or a heterocyclic group: 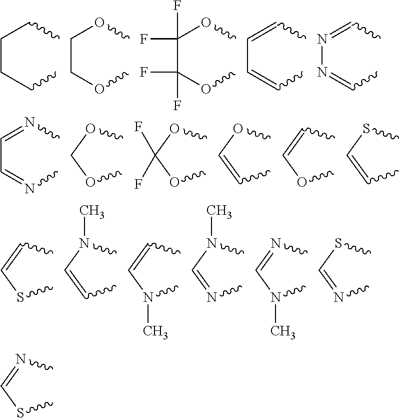
W⁵ represents a nitrogen atom, C—Y¹ or C-(A)r-Q,
W⁶ represents a nitrogen atom, C—Y² or C-(A)r-Q,
W⁷ represents a nitrogen atom, C—Y³ or C-(A)r-Q,
W⁸ represents a nitrogen atom, C—Y⁴ or C-(A)r-Q,
W⁹ represents a nitrogen atom, C—Y⁵ or C-(A)r-Q,
among these, at least one of W⁵ to W⁹ necessarily represents C-(A)r-Q,
Y¹, Y², Y³, Y⁴ and Y⁵, which may be identical or different, represent a hydrogen atom, nitro, formyl, carbamoyl, amino, cyano, halogen, or C₁₋₄ alkyl, C₁₋₄ haloalkyl, C₁₋₄ alkylcarbonyl, C₁₋₄ alkoxycarbonyl, C₁₋₄ acylamino, C₁₋₄ alkoxy, C₁₋₄ haloalkoxy, C₂₋₆ (total number of carbon atoms) alkoxycarbonylalkyl, C₁₋₄ alkylthio, C₁₋₄ alkylsulfinyl, C₁₋₄ alkylsulfonyl, C₁₋₄ haloalkylthio, C₂₋₆ (total number of carbon atoms) alkylthioalkyl, C₁₋₄ monoalkylamino, C₂₋₆ (total number of carbon atoms) dialkylamino, C₁₋₄ monoalkylcarbamoyl, C₂₋₆ (total number of carbon atoms) dialkylcarbamoyl, C₂₋₄ alkenyl, C₂₋₄ alkenyloxy, C₂₋₄ alkynyl, C₂₋₄ alkynyloxy, C₃₋₆ cycloalkyl, C₃₋₆ cycloalkyloxy, C₄₋₇ (total number of carbon atoms) cycloalkylalkyl, C₄₋₇ (total number of carbon atoms) cycloalkylalkoxy which may be substituted, or aryl, aryloxy, arylthio, aryl C₁₋₂ alkyl, aryl C₁₋₂ alkoxy, aryl C₁₋₂ alkylthio, heteroaryl, heterocyclyl, heterocyclyloxy, heterocyclylthio or heterocyclyl C₁₋₂ alkoxy which may be substituted by at least one selected from the group consisting of formyl, cyano, halogen, C₁₋₂ alkyl, C₁₋₂ haloalkyl, C₁₋₂ alkylcarbonyl, C₁₋₆ alkoxycarbonyl, C₁₋₂ alkoxy, C₁₋₂ haloalkoxy, C₁₋₂ alkylthio and C₁₋₂ haloalkylthio, A represents O, S, SO, SO₂, NH, N(CH₃), CH₂, CH(CH₃), CH(C₂H₅), C(═O), C(═N—OCH₃), CH(OH), CH(CN), CH(CF₃), C(CF₃)₂, CH(CO₂CH₃), CH(CO₂C₂H₅), C(═O)CH₂, CH(OH)CH₂ or O(CH₂)₂,
r represents 0, 1, 2 or 3,
Q represents a heterocyclic group of Q1, Q8, Q9, Q11, Q12, Q18, Q20, Q25, Q34, Q35, Q36, Q37, Q38, Q39, Q40, Q41, Q43, Q47, Q48, Q51, Q57 or Q63 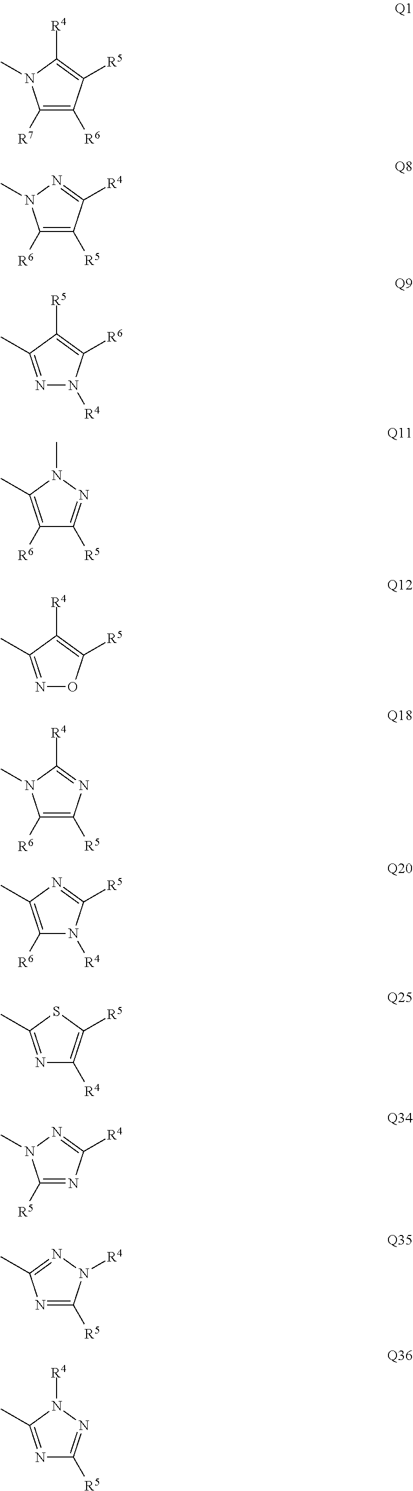 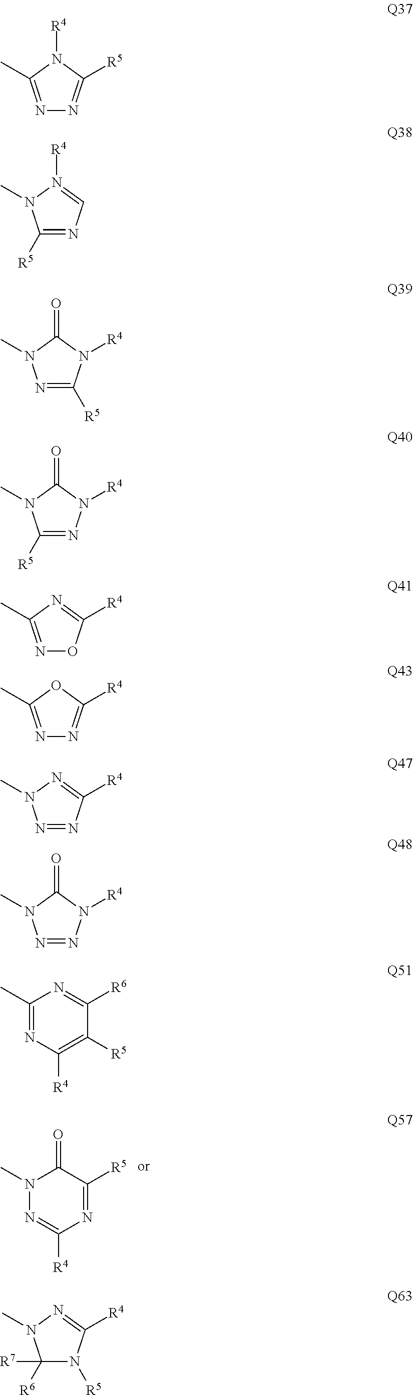
and
R⁴, R⁵, R⁶ and R⁷, which may be identical or different, represent a hydrogen atom, halogen, amino, cyano, nitro, or C₁₋₄ alkyl, C₁₋₈ haloalkyl, C₂₋₄ alkynyl, C₃₋₆ cycloalkyl, C₁₋₄ alkoxy, C₁₋₄ haloalkoxy, C₁₋₄ alkylthio, C₁₋₄ haloalkylthio, C₁₋₄ alkylsulfinyl, C₁₋₄ haloalkylsulfinyl, C₁₋₄ alkylsulfonyl, C₁₋₄ haloalkylsulfonyl, C₁₋₄ alkoxycarbonyl, C₁₋₄ alkylcarbonyl, C₁₋₄ haloalkylcarbonyl, C₁₋₄ acylamino, C₁₋₄ haloacylamino, C₁₋₄ monoalkylcarbamoyl, C₂₋₆ (total number of carbon atoms) dialkylcarbamoyl, hydroxyimino-C₁₋₄ alkyl, C₂₋₆ (total number of carbon atoms) alkoxyiminoalkyl, hydroxyimino-C₁₋₄ haloalkyl, C₂₋₆ (total number of carbon atoms) alkoxyiminohaloalkyl which may be substituted, or phenylcarbamoyl which may be substituted by at least one selected from the group consisting of halogen, cyano, C₁₋₂ alkyl, C₁₋₄ haloalkyl, C₁₋₂ alkoxy, C₁₋₂ haloalkoxy, C₁₋₂ alkylthio and C₁₋₂ haloalkylthio, or a phenyl or heterocyclic group which may be substituted by at least one selected from the group consisting of halogen, cyano, and C₁₋₄ alkyl, C₂₋₄ alkenyl, C₁₋₄ haloalkyl, C₁₋₂ alkoxy, C₁₋₄ haloalkoxy, C₁₋₄ alkylthio, C₁₋₄ haloalkylthio, C₂₋₆ (total number of carbon atoms) alkylthioalkyl, C₁₋₄ alkylcarbonyl and C₁₋₄ alkoxycarbonyl which may be substituted.

4. An insecticide comprising as an active ingredient a compound of claim 1
wherein R¹ represents a hydrogen atom, or alkyl, alkenyl, alkynyl, cycloalkyl, alkoxy, dialkylamino, carboxyalkyl, formylalkyl, hydroxyiminoalkyl, hydroxyalkyl, alkoxyalkyl, aminosulfonylalkyl, alkylaminosulfonylalkyl, alkylthioalkyl, alkylsulfinylalkyl, alkylsulfonylalkyl, cycloalkylthioalkyl, cycloalkylsulfinylalkyl, cycloalkylsulfonylalkyl, trialkylsilylalkyl, alkoxycarbonylmethylaminocarbonylalkyl, alkylcarbamoyloxyalkyl, monoalkylcarbamoylalkyl, monoalkenylcarbamoylalkyl, monoalkynylcarbamoylalkyl, cycloalkylcarbamoylalkyl, alkoxycarbonylaminoalkyl, dialkylaminosulfonylalkyl, benzyloxycarbonylalkyl, benzyloxyalkyl, alkylthioaryl, alkylsulfinylaryl, alkylsulfonylaryl, alkylthioheteroaryl, alkylsulfinylheteroaryl, alkylsulfonylheteroaryl, cycloalkylalkyl, heterocyclyl or heterocyclylalkyl which may be substituted,
R² and R³ each independently represent a hydrogen atom, or alkyl, alkenyl, alkynyl, alkoxyalkyl or alkylthioalkyl which may be substituted,
R¹ and R² together with a nitrogen atom to which they are attached may form a 5- or 6-membered heterocyclic group,
W¹ represents a nitrogen atom or C—X¹,
W² represents a nitrogen atom or C—X²,
W³ represents a nitrogen atom or C—X³,
W⁴ represents a nitrogen atom or C—X⁴,
X¹, X², X³ and X⁴, which may be identical or different, represent a hydrogen atom, nitro, formyl, amino, cyano, halogen, carbamoyl, or alkyl, haloalkyl, alkylcarbonyl, alkoxycarbonyl, acylamino, alkoxy, haloalkoxy, alkylthio, alkylsulfinyl, alkylsulfonyl, haloalkylthio, haloalkylsulfinyl, haloalkylsulfonyl, cycloalkylthio, cycloalkylsulfinyl, cycloalkylsulfonyl, cycloalkylalkylthio, cycloalkylalkylsulfinyl, cycloalkylalkylsulfonyl, alkylsulfonyloxy, haloalkylsulfonyloxy, monoalkylaminosulfonyl, dialkylaminosulfonyl, monoalkylamino, dialkylamino, monoalkylcarbamoyl, dialkylcarbamoyl, alkenyl, alkenyloxy, alkynyl, alkynyloxy, cycloalkyl, cycloalkyloxy, aryl, aryloxy, arylthio, arylalkyl, arylalkoxy, arylalkylthio, heteroaryl, heterocyclyl, heterocyclyloxy, heterocyclylthio or heterocyclylalkoxy which may be substituted, adjacent W¹ and W², W² and W³ or W³ and W⁴ may together form a 5- or 6-membered carbon ring or a heterocyclic group,
W⁵ represents a nitrogen atom, C—Y¹ or C-(A)r-Q,
W⁶ represents a nitrogen atom, C—Y² or C-(A)r-Q,
W⁷ represents a nitrogen atom, C—Y³ or C-(A)r-Q,
W⁸ represents a nitrogen atom, C—Y⁴ or C-(A)r-Q,
W⁹ represents a nitrogen atom, C—Y⁵ or C-(A)r-Q,
at least one of W⁵ to W⁹ necessarily represents C-(A)r-Q, Y¹, Y², Y³, Y⁴ and Y⁵, which may be identical or different, represent a hydrogen atom, nitro, formyl, carbamoyl, amino, cyano, halogen, or alkyl, haloalkyl, alkylcarbonyl, alkoxycarbonyl, acylamino, alkoxy, haloalkoxy, alkoxycarbonylalkyl, alkylthio, alkylsulfinyl, alkylsulfonyl, haloalkylthio, alkylthioalkyl, monoalkylamino, dialkylamino, monoalkylcarbamoyl, dialkylcarbamoyl, alkenyl, alkenyloxy, alkynyl, alkynyloxy, cycloalkyl, cycloalkyloxy, cycloalkylalkyl, cycloalkylalkoxy, aryl, aryloxy, arylthio, arylalkyl, arylalkoxy, arylalkylthio, heteroaryl, heterocyclyl, heterocyclyloxy, heterocyclylthio or heterocyclylalkoxy which may be substituted,
A represents O, S, SO, SO₂, NH, N(CH₃), CH(CN), C(═N—OCH₃), C(═O), CH(OH), CH(CH₃), CH(CF₃), C(CF₃)₂, CH(CO₂CH₃) or CH(CO₂C₂H₅), or alkylene which may be interrupted by O, S, SO, SO₂, NH, N(CH₃), CH(CN), C(═N—OCH₃), C(═O), CH(OH), CH(CH₃), CH(CF₃), C(CF₃)₂, CH(CO₂CH₃) or CH(CO₂C₂H₅),
r represents 0, 1, 2, 3, 4 or 5, and
Q represents a 5- or 6-membered heterocyclic group which may be substituted.
